(19) United States
(12) Patent Application Publication
(10) Pub. No.: US 2009/0270350 A1
(43) Pub. Date: Oct. 29, 2009

(54) Pyridinone Pyrazole Urea and Pyrimidinone Pyrazole Urea Derivatives

(75) Inventors: Balekudru Devadas, Chesterfield, MO (US); Susan J. Hartmann, Kirkwood, MO (US); Richard F. Heier, Columbia, IL (US); Kevin D. Jerome, St. Charles, MO (US); Steve A. Kolodziej, Ballwin, MO (US); John Mathias, Kent (GB); Monica B. Norton, Chesterfield, MO (US); Michele A. Promo, Ballwin, MO (US); Paul V. Rucker, Carlsbad, CA (US); Shaun Selness, Chesterfield, MO (US)

Correspondence Address: PFIZER INC., PATENT DEPARTMENT, MS8260-1611, EASTERN POINT ROAD, GROTON, CT, 06340, US

(73) Assignee: PFIZER INC.

(21) Appl. No.: 12/162,048

(22) PCT Filed: Feb. 5, 2007
(86) PCT No.: PCT/IB2007/000380

§ 371 Date: Jul. 24, 2008

Related U.S. Application Data
(60) Provisional application No. 60/772,262, filed on Feb. 10, 2006.

(51) Int. Cl. A61K 31/695; C07F 7/18; A61K 31/4439; C07D 401/12

(52) U.S. Cl. 514/63; 546/14; 514/341; 546/275.4

(57) ABSTRACT

This invention is directed generally to substituted pyridinone and pyrimidinone compounds that generally inhibit p38 kinase, TNF, and/or cyclooxygeπase activity. Such substituted pyridinone and pyrimidinone compounds include compounds generally corresponding in structure to the following formula: wherein Z, n, R¹, R^{2a}, R^{2b}, R^{2c}, R^{2d}, R^{2e}, R^{3a}, R^{3b}, R^{3c}, R^{3d}, R⁴, R⁵, R⁶, R^{7a}, R^{7b}, R^{7c}, R^{7d} and R^{7e} are as defined in this specification. This invention also is directed to compositions of such substituted pyridinones and pyrimidinones (particularly pharmaceutical compositions), and methods for treating disorders (typically pathological disorders) associated with p38 kinase activity, TNF activity, and/or cyclooxygenase-2 activity.

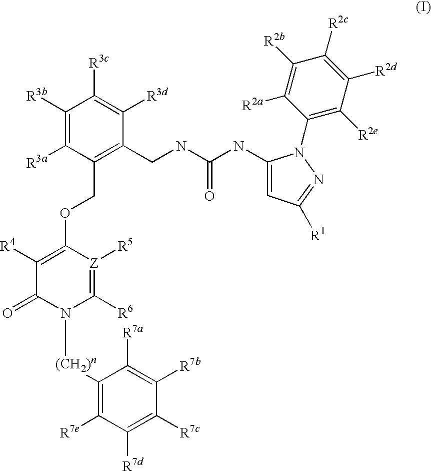

### FIELD OF THE INVENTION

This invention is directed to compounds that inhibit p38 kinase (particularly p38α kinase), TNF (particularly TNF-α), and/or cyclooxygenase (particularly cyclooxygenase-2 or “COX-2”) activity. This invention also is directed to compositions of such compounds, methods for making such compounds, and methods for treating disorders (typically pathological disorders) associated with p38 kinase activity, TNF activity, and/or cyclooxygenase-2 activity.

### BACKGROUND OF THE INVENTION

Mitogen-activated protein kinases (MAP) constitute a family of proline-directed serine/threonine kinases that activate their substrates by dual phosphorylation. The kinases are activated by a variety of signals, including nutritional and osmotic stress, UV light, growth factors, endotoxin, and inflammatory cytokines. The p38 MAP kinase group is a MAP family of various isoforms, including p38α, p38β, and p38γ. These kinases are responsible for phosphorylating and activating transcription factors (e.g., ATF2, CHOP, and MEF2C), as well as other kinases (e.g., MAPKAP-2 and MAPKAP-3). The p38 isoforms are activated by bacterial lipopolysaccharide, physical and chemical stress, and pro-inflammatory cytokines, including tumor necrosis factor (“TNF”) and interleukin-1 (“IL-1”). The products of the p38 phosphorylation mediate the production of inflammatory cytokines, including TNF, IL-1, and cyclooxygenase-2.

It is believed that p38α kinase can cause or contribute to the effects of, for example, inflammation generally; arthritis; neuroinflammation; pain; fever; pulmonary disorders; cardiovascular diseases; cardiomyopathy; stroke; ischemia; reperfusion injury; renal reperfusion injury; brain edema; neurotrauma and brain trauma; neurodegenerative disorders; central nervous system disorders; liver disease and nephritis; gastrointestinal disorders; ulcerative diseases; ophthalmic diseases; opthalmological disorders; glaucoma; acute injury to the eye tissue and ocular traumas; diabetes; diabetic nephropathy; skin-related disorders; viral and bacterial infections; myalgias due to infection; influenza; endotoxic shock; toxic shock syndrome; autoimmune disease; bone resorption diseases; multiple sclerosis; disorders of the female reproductive system; pathological (but non-malignant) disorders, such as hemaginomas, angiofibroma of the nasopharynx, and avascular necrosis of bone; benign and malignant tumors/neoplasia including cancer; leukemia; lymphoma; systemic lupus erthrematosis (SLE); angiogenesis including neoplasia; and metastasis.

TNF is a cytokine produced primarily by activated monocytes and macrophages. Excessive or unregulated TNF production (particularly TNF-α) has been implicated in mediating a number of diseases. It is believed, for example, that TNF can cause or contribute to the effects of inflammation (e.g., rheumatoid arthritis and inflammatory bowel disease), asthma, autoimmune disease, graft rejection, multiple sclerosis, fibrotic diseases, cancer, fever, psoriasis, cardiovascular diseases (e.g., post-ischemic reperfusion injury and congestive heart failure), pulmonary diseases (e.g., hyperoxic alveolar injury), hemorrhage, coagulation, radiation damage, and acute phase responses like those seen with infections and sepsis and during shock (e.g., septic shock and hemodynamic shock). Chronic release of active TNF can cause cachexia and anorexia. And TNF can be lethal.

TNF also has been implicated in infectious diseases. These include, for example, malaria, mycobacterial infection and meningitis. These also include viral infections, such as HIV, influenza virus, and herpes virus, including herpes simplex virus type-1 (HSV-1), herpes simplex virus type-2 (HSV-2), cytomegalovirus (CMV), varicella-zoster virus (VZV), Epstein-Barr virus, human herpesvirus-6 (HHV-6), human herpesvirus-7 (HHV-7), human herpesvirus-8 (HHV-8), pseudorabies and rhinotracheitis, among others.

IL-8 is another pro-inflammatory cytokine, which is produced by mononuclear cells, fibroblasts, endothelial cells, and keratinocytes. This cytokine is associated with disorders including inflammation.

IL-1 is produced by activated monocytes and macrophages, and is involved in inflammatory responses. IL-1 plays a role in many pathophysiological responses, including rheumatoid arthritis, fever, and reduction of bone resorption.

TNF, IL-1, and IL-8 affect a wide variety of cells and tissues, and are important inflammatory mediators of a wide variety of disorders. The inhibition of these cytokines by inhibition of the p38 kinase is beneficial in controlling, reducing, and alleviating many of these disease states.

Various substituted pyridinones and pyrimidinones have previously been described:

WO03/068,230 published on Aug. 21, 2003 refers to certain substituted pyridinones.

WO04/087677 published on Oct. 14, 2004 refers to certain substituted pyrimidinones.

U.S. patent application Ser. No. 10/808,146 (filed Mar. 24, 2004) refers to certain substituted pyrimidinones.

PCT application number PCT/IB05/003063 filed Oct. 3, 2005 refers to certain substituted pyrimidinones.

PCT application number PCT/IB05/002574 filed Aug. 9, 2005 refers to certain pyrazolyl-3-[2-(triazolopyridinylsulfanyl)-benzyl]-urea derivatives.

There is a need to provide new p38 kinase inhibitors that are good drug candidates. Preferably, p38 kinase inhibitors that show good potency, high levels of selectivity over other related protein kinases, have properties particularly suitable for providing effective treatment via the inhalation route, are suitable for the treatment of allergic and non-allergic airways diseases (particularly obstructive or inflammatory airways diseases), are non-toxic and demonstrate few side-effects, have physical properties suitable for administration by inhalation, exist in a physical form that is stable and non-hygroscopic, and/or are easily formulated. The following disclosure describes substituted pyridinone and pyrimidinone compounds that exhibit one or more such desirable qualities.

### SUMMARY OF THE INVENTION

This invention is directed to substituted pyridinone pyrazole urea and pyrimidinone pyrazole urea compounds that inhibit p38 kinase activity, TNF activity, and/or cyclooxygenase-2 activity. This invention also is directed to, for example, a method for inhibiting p38 kinase, TNF, and/or cyclooxygenase-2 activity, and particularly to a method for treating a disorder (typically a pathological disorder) mediated by p38 kinase activity, TNF activity, and/or cyclooxygenase-2 activity. Such a method is typically suitable for use with mammals in need of such treatment.

Briefly, therefore, this invention is directed, in part, to compounds of formula I:

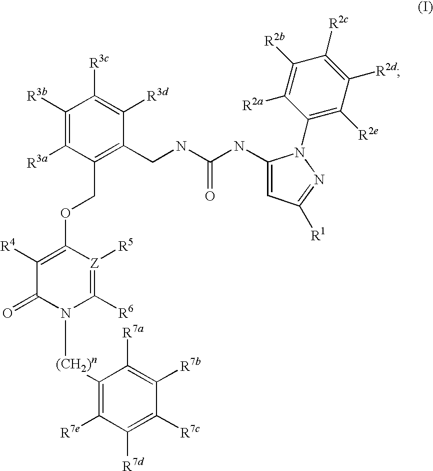

or a pharmaceutically acceptable salt, enantiomer or racemate thereof, wherein;

Z is C or N;

n is 0 or 1;

R¹ is (C₁-C₄)-alkyl, (C₃-C₆)-cycloalkyl, or [(C₁-C₄)-alkyl)][(C₁-C₄)-alkyl-S]-(C₁-C₄)-alkyl;

R^{2a}, R^{2b}, R^{2c}, R^{2d} and R^{2e} are independently H, (C₁-C₄)-alkyl, —OH, halo, (C₁-C₄)-alkyl-O—, heterocyclyloxy-(C₁-C₄)-alkyl-O—, OH—(C₁-C₄)-alkyl-O—, or (C₁-C₄)-alkyl[(C₁-C₄)-dialkyl]silyloxy;

R^{3a}, R^{3b}, R^{3c} and R^{3d} are independently —H or -halo;

R⁴ is H, halo or (C₁-C₄)-alkyl;

R⁵ is —H or absent when Z is N;

R⁶ is —H, (C₁-C₄)-alkyl or (C₁-C₄)-alkyl-S— and;

R^{7a}, R^{7b}, R^{7c}, R^{7d} and R^{7e} are independently —H, —C(O)—O—(C₁-C₄)-alkyl, (C₁-C₄)-alkyl-S—, (C₁-C₄)-alkoxy, —OH, NH₂—(C₁-C₄)-alkyl-C(O)—NH—(C₁-C₄)-alkyl, (C₁-C₄)-alkyl-O—C(O)—NH—(C₁-C₄)-alkyl-C(O)—NH—(C₁-C₄)-alkyl-, halo,

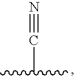

OH—(C₁-C₄)-alkyl-C(O)—NH—(C₁-C₄)-alkyl, —(C₁-C₄)-alkyl, —C(O)—OH, OH—(C₁-C₄)-alkyl-NH—C(O), (C₁-C₄)-alkyl-NH—C(O)—, (OH)₂—(C₁-C₄)-alkyl-NH—C(O)—, (C₁-C₄)-alkoxy-(C₁-C₄)-alkyl-NH—C(O)—, NH₂—C(O)—(C₁-C₄)-alkyl-NH—C(O)— (C₁-C₄)-alkyl-NH—C(O)—(C₁-C₄)-alkyl-NH—C(O)—, (C₁-C₄)-dialkyl-NH—(C₁-C₄)-alkyl-NH—C(O)—, NH₂—C(O)—NH—(C₁-C₄)-alkyl-NH—C(O)—, or [NH₂—C(O)][(C₁-C₄)-alkyl](C₁-C₄)-alkyl-NH—C(O)— or pharmaceutically acceptable salt thereof.

This invention also is directed, in part, to pharmaceutical compositions comprising a therapeutically-effective amount of an above-described compound or pharmaceutically acceptable salt thereof.

This invention is also directed, in part, to methods for treating allergic and non-allergic airways diseases, more particularly obstructive or inflammatory airways diseases such as chronic obstructive pulmonary disease (“COPD”) in a mammal.

This invention is also directed to methods for treating asthma comprising administering to a subject in need of such treatment, an effective amount of a compound of the present invention, or a pharmaceutically acceptable salt thereof.

This invention also is directed, in part, to a method for treating an inflammatory disorder in a mammal. The method comprises administering an above-described compound or pharmaceutically acceptable salt thereof, to the mammal in an amount that is therapeutically-effective to treat the disorder.

Further benefits of Applicants' invention will be apparent to one skilled in the art from reading this specification.

### DETAILED DESCRIPTION

### Definitions

The following is a list of definitions of carious terms used herein:

The term “alkyl” refers to a straight chain or branched chain hydrocarbon radical having from about 1 to about 10 carbon atoms, and in another embodiment from 1 to about 6 carbon atoms. Examples of such alkyl radicals are methyl, ethyl, n-propyl, isopropyl, n-butyl, isobutyl, sec-butyl, t-butyl, pentyl, neopentyl, hexyl, isohexyl, and the like.

The term “alkoxy” means an alkylether substituent, i.e., —O-alkyl. Examples of such a substituent include —O—CH₃, —O—CH₂—CH₃, n-propoxy, isopropoxy, n-butoxy, iso-butoxy, sec-butoxy, and tert-butoxy.

The term “alkylcarbonyl” or “alkanoyl” means —C(O)-alkyl. For example, “ethylcarbonyl” may be depicted as:

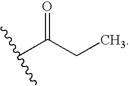

In other examples in the present invention, alkylcarbonyl substituents include methylcarbonyl, propylcarbonyl, butylcarbonyl, pentylcarbonyl, and hexylcarbonyl.

The term “alkoxycarbonyl” means —C(O)—O-alkyl. For example, “ethoxycarbonyl” may be depicted as:

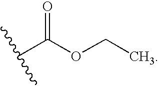

Examples of other alkoxycarbonyl substituents of the present invention include —C(O)—O—CH₃, ethoxycarbonyl, propoxycarbonyl, butoxycarbonyl, pentoxycarbonyl, and hexyloxycarbonyl.

The term “amino” means —NH₂. The term “monosubstituted amino” means an amino substituent wherein one of the hydrogen radicals is replaced by a non-hydrogen substituent. The term “disubstituted amino” means an amino substituent wherein both of the hydrogen atoms are replaced by non-hydrogen substituents, which may be identical or different.

The term “aminocarbonyl” means —C(O)—NH₂, which also may be depicted as:

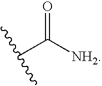

The term “cycloalkyl” means a saturated carbocyclyl substituent containing from 3 to about 14 carbon ring atoms, more typically from 3 to about 12 carbon ring atoms, and even more typically from 3 to about 8 carbon ring atoms. A cycloalkyl may be a single carbon ring, which typically contains from 3 to 6 carbon ring atoms. Examples of single-ring cycloalkyls include cyclopropyl, cyclobutyl, cyclopentyl and cyclohexyl.

The term “aryl” means an aromatic carbocyclyl containing from 6 to 14 carbon ring atoms. Examples of aryls include phenyl, naphthalenyl, and indenyl.

The term “arylalkyl” means alkyl substituted with aryl.

The term “carboxy” or “carboxyl” means —C(O)—OH, which also may be depicted as:

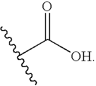

The term “carbonyl” means —C(O)—, which also may be depicted as:

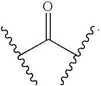

This term also is intended to encompass a hydrated carbonyl substituent, i.e., —C(OH)₂—.

The term “nitro” (alone or in combination with another term(s)) means —NO₂.

The term “cyano” (alone or in combination with another term(s)) means —CN, which also may be depicted:

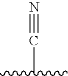

The term “keto” (alone or in combination with another term(s)) means an oxo radical, and may be depicted as ═O.

The term “hydrogen” means a hydrogen radical, and may be depicted as —H.

The term “hydroxy” or “hydroxyl” means —OH.

The term “hydroxyalkyl” means alkyl substituted with one more hydroxy.

The term “halo” or “halogen” refers to bromo, chloro, fluoro or iodo.

The term “oxy” means an ether substituent, and may be depicted as —O—.

The term “thioalkyl” a thio substituted alkyl, which is also depicted as:

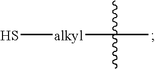

Examples of such substituents are thiomethyl, thioethyl and thiobutyl.

The term “heterocyclyl” means a saturated (i.e., “heterocycloalkyl”), partially saturated (i.e., “heterocycloalkenyl”), or completely unsaturated (i.e., “heteroaryl”) ring structure containing a total of 3 to 14 ring atoms. At least one of the ring atoms is a heteroatom (i.e., oxygen, nitrogen, or sulfur), with the remaining ring atoms being independently from the group consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, pyrrolyl, pyrrolinyl, pyrrolidinyl, imidazolyl, pyrazolyl, pyrazolinyl, pyrazolidinyl, triazolyl, oxazolyl, thiazolyl, pyranyl pyridinyl, piperidinyl, diazinyl, pyrimidinyl, piperazinyl, morpholinyl, thiomorpholinyl, thiomorpholinyl S-oxide and thiomorpholinyl S,S-dioxide.

The term “heteroaryl” means an aromatic heterocyclyl containing from 5 to 14 ring atoms. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as 1,2-, 1,4-, 2,3- and 2,1-benzopyronyl, quinolinyl, isoquinolinyl, cinnolinyl, quinazolinyl, and 1,4-benzoxazinyl.

The term [alkyl][alkylthio]alkyl is depicted as:

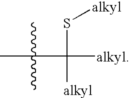

An example of such substituents is [methyl][methylthio]ethyl which is depicted as:

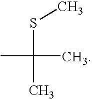

The term alkyl[dialkyl]silyloxy is depicted as:

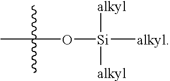

An example of such substituent would be tert-butyl[dimethyl]silyloxy which is depicted as:

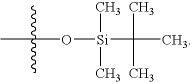

In the alternative, this substitutent could be named aminocarbonylalkylaminocarbonyl.

The term [aminocarbonyl][alkyl]alkylaminocarbonyl is depicted as:

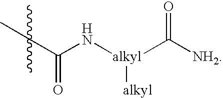

An example of such substituent would be [aminocarbonyl][methyl]methylaminocarbonyl which is depicted as:

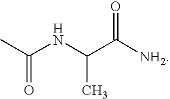

In the alternative, this substituent could be named aminocarbonylethylaminocarbonyl.

The terms “substituent” and “radical” are interchangeable. If substituents are described as being “independently” from a group, each substituent is independent of the other. Each substituent therefore may be identical to or different from the other substituent(s).

The term “pharmaceutically-acceptable” is used adjectivally in this specification to mean that the modified noun is appropriate for use as a pharmaceutical product or as a part of a pharmaceutical product. With reference to the use of the words “comprise” or “comprises” or “comprising” in this patent (including the claims), Applicants note that unless the context requires otherwise, those words are used on the basis and clear understanding that they are to be interpreted inclusively, rather than exclusively, and that Applicants intend each of those words to be so interpreted in construing this patent, including the claims below.

The term “treatment”, as used herein to describe the present invention and unless otherwise qualified, means administration of the compound, pharmaceutical composition or combination to effect preventative, palliative, supportive, restorative or curative treatment.

The term “preventive treatment,” as used herein to describe the present invention, means that the compound, pharmaceutical composition or combination is administered to a subject to inhibit or stop the relevant disorder from occurring in a subject, particularly in a subject or member of a population that is significantly predisposed to the relevant disorder.

The term “palliative treatment,” as used herein to describe the present invention, means that the compound, pharmaceutical composition or combination is administered to a subject to remedy signs and/or symptoms of a disorder, without necessarily modifying the progression of, or underlying etiology of, the relevant disorder. Non-limiting examples include reduction in pain, discomfort, swelling or fever.

The term “supportive treatment,” as used herein to describe the present invention, means that the compound, pharmaceutical composition or combination is administered to a subject as a part of a regimen of therapy, but that such therapy is not limited to administration of the compound, pharmaceutical composition or combination. Non-limiting examples include administration of the compound or combination to a subject simultaneously with, prior to, or subsequent to surgery; administration of the compound or combination with a further combination of drugs or agents; and administration of the compound or combination simultaneously with, prior to or subsequent to radiation therapy. Unless otherwise expressly stated, supportive treatment may embrace preventive, palliative, restorative or curative treatment, particularly when the compounds or pharmaceutical compositions are combined with another component of supportive therapy.

The term “restorative treatment,” as used herein to describe the present invention, means that the compound, pharmaceutical composition or combination is administered to a subject to modify the underlying progression or etiology of a disorder. Non-limiting examples include increase in forced expiratory volume in one second (FEV 1) for lung disorders, inhibition of progressive nerve destruction, reduction of biomarkers associated and correlated with diseases or disorders, and the like.

The term “curative treatment,” as used herein to describe the present invention, means that compound, pharmaceutical composition or combination is administered to a subject for the purpose of bringing the disease or disorder into complete remission, or that the disease or disorder is undetectable after such treatment.

This detailed description of embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating embodiments of this invention, are intended for purposes of illustration only. This invention, therefore, is not limited to the embodiments described in this specification, and may be variously modified.

### COMPOUNDS OF THIS INVENTION

In accordance with this invention, it has been found that certain substituted pyridinone pyrazole urea and pyrimidinone pyrazole urea compounds are effective for inhibiting the activity (particularly pathological activity) of p38 kinase, TNF, and/or cyclooxygenase-2.

Among its many embodiments, the present invention provides a compound of Formula I:

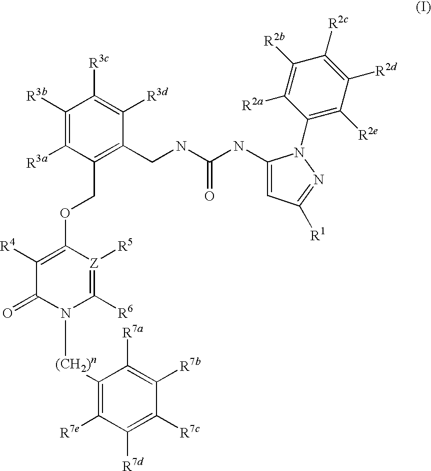

wherein;

Z is C or N;

n is 0 or 1;

R¹ is (C₁-C₄)-alkyl, (C₃-C₆)-cycloalkyl, or [(C₁-C₄)-alkyl)][(C₁-C₄)-alkyl-S]—(C₁-C₄)-alkyl; R^{2a}, R^{2b}, R^{2c}, R^{2d} and R^{2e} are independently H, (C₁-C₄)-alkyl, —OH, halo, (C₁-C₄)-alkoxy, heterocyclyloxy-(C₁-C₄)-alkoxy, OH—(C₁-C₄)-alkoxy, or (C₁-C₄)-alkyl[(C₁-C₄)-dialkyl]silyloxy;

R^{3a}, R^{3b}, R^{3c} and R^{3d} are independently H or halo;

R⁴ is —H, -halo or —(C₁-C₄)-alkyl;

R⁵ is —H or absent when Z is N;

R⁶ is —H, —(C₁-C₄)-alkyl or —(C₁-C₄)-alkyl-S—; and

R^{7a}, R^{7b}, R^{7c}, R^{7d} and R^{7e} are independently H, —C(O)—O—(C₁-C₄)-alkyl, (C₁-C₄)-alkyl-S—, (C₁-C₄)-alkyl-O—, —OH, NH₂—(C₁-C₄)-alkyl-C(O)—NH—C₁-C₄)-alkyl-, (C₁-C₄)-alkyl-O—C(O)—NH—,

—(C₁-C₄)-alkyl-C(O)—NH—(C₁-C₄)-alkyl —C(O)—O-alkyl, halo,

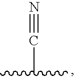

OH—(C₁-C₄)-alkyl-C(O)—NH—(C₁-C₄)-alkyl, (C₁-C₄)-alkyl, —C(O)—OH, OH—(C₁-C₄)-alkyl-NH—C(O), (C₁-C₄)-alkyl-NH—C(O)—, (OH)₂—(C₁-C₄)-alkyl-NH—C(O)—, (C₁-C₄)-alkoxy-(C₁-C₄)-alkyl-NH—C(O)—, NH₂—C(O)—(C₁-C₄)-alkyl-NH—C(O)—, (C₁-C₄)-alkyl-NH—C(O)—(C₁-C₄)-alkyl-NH—C(O)—, (C₁-C₄)-dialkyl-NH—(C₁-C₄)-alkyl-NH—C(O)—, or NH₂—C(O)—(C₁-C₄)-alkyl](C₁-C₄)-alkyl-NH—C(O)—.

In another embodiment, the present invention provides a compound of Formula I:

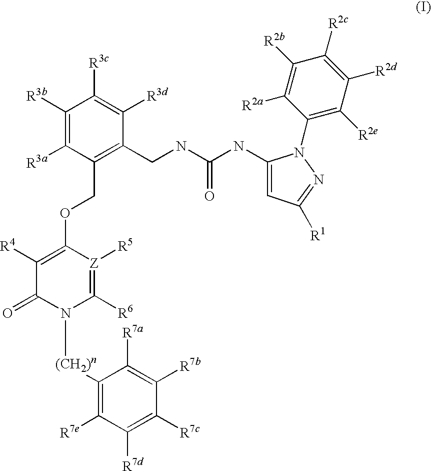

wherein;

Z is C or N;

n is 0 or 1;

R¹ is —C—(CH₃)₃,

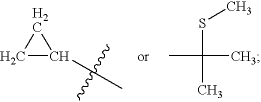

R^{2a}, R^{2b}, R^{2c}, R^{2d} and R^{2e} are independently —H, —Cl, —CH₃, —OH, —O—CH₃, OH—CH₂—CH₂—O—, —F, difluoropyranyloxyethoxy or tert-butyl[dimethyl]silyloxy;

R^{3a}, R^{3b}, R^{3c} and R^{3d} are independently —H or —F;

R⁴ is —H, —Br, —Cl, —CH₃ or —CH₂—CH₃;

R⁵ is —H or absent when Z is N;

R⁶ is —H, —CH₃ or CH₃—S—; and

R^{7a}, R^{7b}, R^{7c}, R^{7d} and R^{7e} are independently —H, —C(O)—O—CH₃, CH₃—S—, —O—CH₃, —OH, NH₂—CH₂—C(O)—NH—CH₂—, C(CH₃)₃—C(O)—NH—CH₂—C(O)—NH—CH₂—Cl,

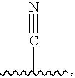

### OH—CH₂—C(O)—NH—CH₂—, —CH₃, —C(O)—OH,

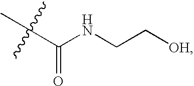

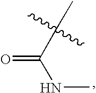

### (OH)₂—(CH₂)₃—NH—C(O)—,

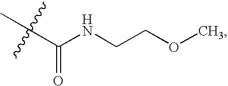

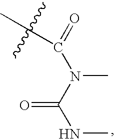

### NH₂—C(O)—CH₂—NH₂—C(O)—,

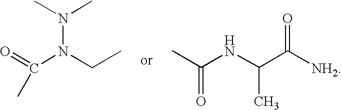

In one embodiment, the present invention provides a compound of Formula II:

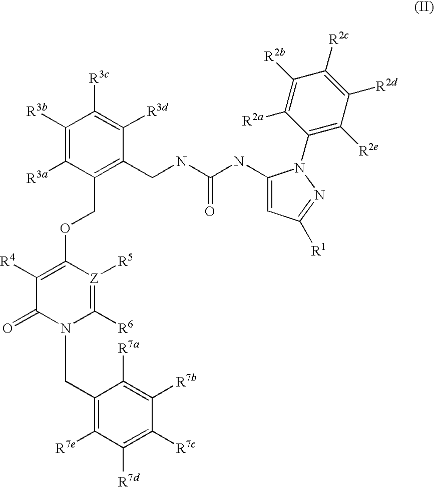

wherein;

Z is C or N;

R¹ is (C₁-C₄)-alkyl, (C₃-C₆)-cycloalkyl, or [(C₁-C₄)-alkyl)][(C₁-C₄)-alkyl-S]—(C₁-C₄)-alkyl;

R^{2a}, R^{2b}, R^{2c}, R^{2d} and R^{2e} are independently —H, (C₁-C₄)-alkyl, OH—, halo, (C₁-C₄)-alkyl-O—, heterocyclyloxy-(C₁-C₄)-alkyl-O—, OH—(C₁-C₄)-alkyl-O—, or (C₁-C₄)-alkyl[(C₁-C₄)-dialkyl]silyloxy;

R^{3a}, R^{3b}, R^{3c} and R^{3d} are independently —H or halo;

R⁴ is —H, halo or (C₁-C₄)-alkyl;

R⁵ is —H or absent when Z is N;

R⁶ is —H or (C₁-C₄)-alkyl; and

R^{7a}, R^{7b}, R^{7c}, R^{7d} and R^{7e} are independently —H, —C(O)—O—(C₁-C₄)-alkyl, (C₁-C₄)-alkyl-S—, (C₁-C₄)-alkyl-O—, OH—, NH₂—(C₁-C₄)-alkyl-C(O)—NH—(C₁-C₄)-alkyl-, (C₁-C₄)-alkyl-O—C(O)—NH—, (C₁-C₄)-alkyl-C(O)—NH—(C₁-C₄)-alkyl-, -halo,

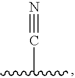

or OH—(C₁-C₄)-alkyl-C(O)—NH—(C₁-C₄)-alkyl-.

In another embodiment, the present invention provides a compound of Formula II; wherein;

Z is —C or —N;

R¹ is —C—(CH₃)₃;

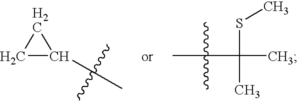

R^{2a}, R^{2b}, R^{2c}, R^{2d} and R^{2e} are independently —H, —Cl, —CH₃, —OH, —O—CH₃, OH—CH₂—CH₂—O—, —F, difluoropyranyloxyethoxy or tert-butyl[dimethyl]silyloxy;

R^{3a}, R^{3b}, R^{3c} and R^{3d} are independently —H or —F;

R⁴ is —H, —Br, —Cl, —CH₃ or —CH₂—CH₃;

R⁵ is —H or absent when Z is N;

R⁶ is —H or —CH₃; and

R^{7a}, R^{7b}, R^{7c}, R^{7d} and R^{7e} are independently —H, —C(O)—O—CH₃, CH₃—S—, —O—CH₃, OH—, NH₂—CH₂—C(O)—NH—CH₂—,

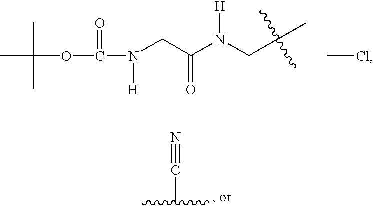

### OH—CH2-C(O)—NH—CH2-

In one embodiment, the present invention provides a compound of Formula III:

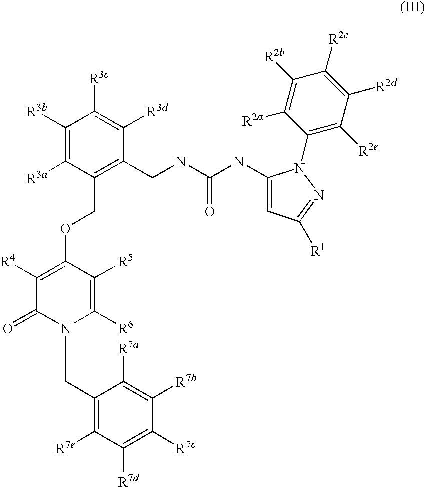

wherein;

R¹ is (C₁-C₄)-alkyl, (C₃-C₆)-cycloalkyl, or [(C₁-C₄)-alkyl)][(C₁-C₄)-alkyl-S]—(C₁-C₄)-alkyl;

R^{2a}, R^{2b}, R^{2c}, R^{2d} and R^{2e} are independently —H, (C₁-C₄)-alkyl, OH—, halo, (C₁-C₄)-alkyl-O—, heterocyclyloxy-(C₁-C₄)-alkyl-O—, OH—(C₁-C₄)-alkyl-O—, or (C₁-C₄)-alkyl[(C₁-C₄)-dialkyl]silyloxy;

R^{3a}, R^{3b}, R^{3c} and R^{3d} are independently —H or halo;

R⁴ is —H, halo or (C₁-C₄)-alkyl;

R⁵ is —H;

R⁶ is (C₁-C₄)-alkyl; and

R^{7a}, R^{7b}, R^{7c}, R^{7d} and R^{7e} are independently H, —C(O)—O—(C₁-C₄)-alkyl, (C₁-C₄)-alkyl-S—, (C₁-C₄)-alkyl-O—, OH—, NH₂—(C₁-C₄)-alkyl-C(O)—NH—(C₁-C₄)-alkyl, (C₁-C₄)-alkyl-O—C(O)—NH—, —(C₁-C₄)-alkyl-C(O)—NH— (C₁-C₄)-alkyl, halo,

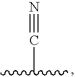

or OH—(C₁-C₄)-alkyl-C(O)—NH—(C₁-C₄)-alkyl.

In another embodiment, the present invention provides a compound of Formula III:

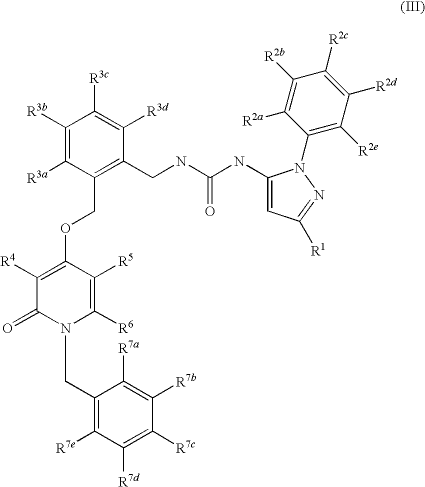

wherein;

R¹ is —C—(CH₃)₃,

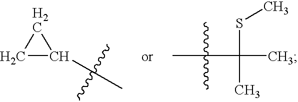

R^{2a}, R^{2b}, R^{2c}, R^{2d} and R^{2e} independently —H, —Cl, —CH₃, OH—, —O—CH₃, OH—CH₂—CH₂—O—, —F, difluoropyranyloxyethoxy or

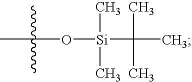

R^{3a}, R^{3b}, R^{3c} and R^{3d} are independently —H or —F;

R⁴ is —H, —Br, —Cl or —CH₃;

R⁵ is —H;

R⁶ is —CH₃; and

R^{7a}, R^{7b}, R^{7c}, R^{7d} and R^{7e} are independently —H, —C(O)—O—CH₃, CH₃—S—, —O—CH₃, OH—, NH₂—CH₂—C(O)—NH—CH₂—,

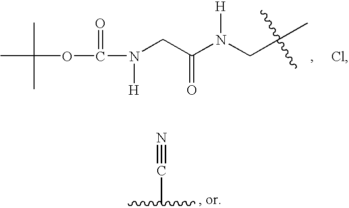

### OH—CH2-C(O)—NH—CH2-.

In one embodiment, the present invention provides a compound of Formula IV:

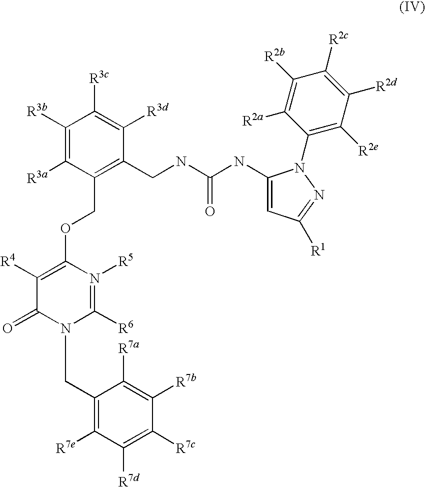

wherein;
R¹ is (C₁-C₄)-alkyl;
R^{2a}, R^{2b}, R^{2c}, R^{2d} and R^{2e} are independently —H, —(C₁-C₄)-alkyl, -halo or (C₁-C₄)-alkyl-O—;
R^{3a}, R^{3b}, R^{3c} and R^{3d} are —H;
R⁴ is —(C₁-C₄)-alkyl;
R⁵ is —H;
R⁶ is —H; and
R^{7a}, R^{7b}, R^{7c}, R^{7d} and R^{7e} are independently —H or (C₁-C₄)-alkyl-O.

In another embodiment, the present invention provides a compound of Formula IV:

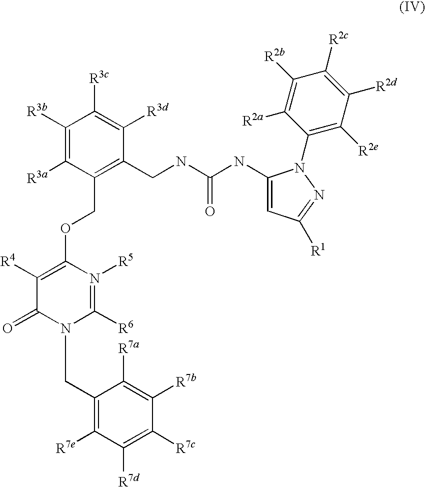

wherein;

R¹ is —C—(CH₃)₃;

R^{2a}, R^{2b}, R^{2c}, R^{2d} and R^{2e} are independently —H, —CH₃, —F or —O—CH₃;

R^{3a}, R^{3b}, R^{3c} and R^{3d} are —H;

R⁴ is —CH₂—CH₃;

R⁵ is —H;

R⁶ is —H; and

R^{7a}, R^{7b}, R^{7c}, R^{7d} and R^{7e} are independently —H or —O—CH₃.

In one embodiment, the present invention provides a compound of Formula V:

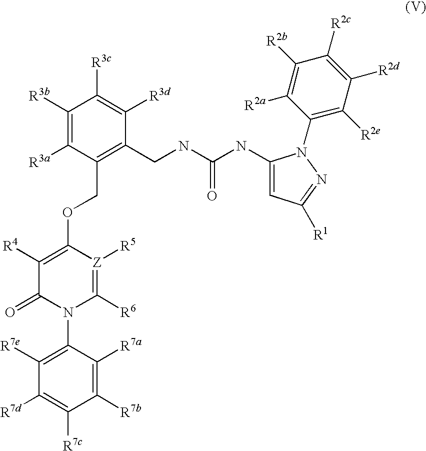

wherein;

Z is —C or —N;

R¹ is —(C₁-C₄)-alkyl;

R^{2a}, R^{2b}, R^{2c}, R^{2d} and R^{2e} are independently —H, —(C₁-C₄)-alkyl, OH—, -halo, (C₁-C₄)-alkyl-O— or OH—(C₁-C₄)-alkyl-O—;

R^{3a}, R^{3b}, R^{3c} and R^{3d} are independently —H or -halo;

R⁴ is —H or -halo;

R⁵ is —H or absent when Z is N;

R⁶ is (C₁-C₄)-alkyl or (C₁-C₄)-alkyl-S—; and

R^{7a}, R^{7b}, R^{7c}, R^{7d} and R^{7e} are independently —H, —C(O)—O—(C₁-C₄)-alkyl, (C₁-C₄)-alkyl, —C(O)—OH, OH—(C₁-C₄)-alkyl-NH—C(O)—, (C₁-C₄)-alkyl-NH—C(O)—, (OH)₂—(C₁-C₄)-alkyl-NH—C(O)—, (C₁-C₄)-alkyl-O—(C₁-C₄)-alkyl-NH—C(O)—, NH₂—C(O)—(C₁-C₄)-alkyl-NH—C(O)—, (C₁-C₄)-alkyl-NH—C(O)—(C₁-C₄)-alkyl-NH—C(O)—, (C₁-C₄)-dialkyl-N—(C₁-C₄)-alkyl-NH—C(O)— or [NH₂—C(O)][(C₁-C₄)-alkyl](C₁-C₄)-alkyl-NH—C(O)—.

In another embodiment, the present invention provides a compound of Formula V:

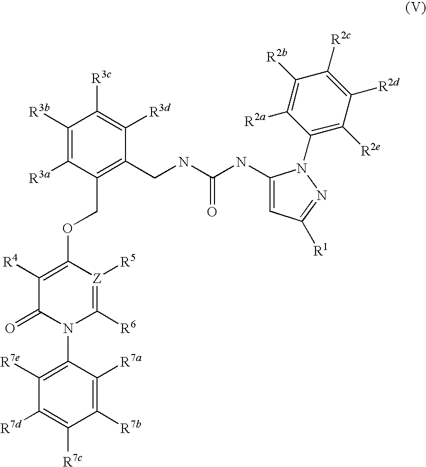

wherein;

Z is —C or —N;

R¹ is —C—(CH₃)₃;

R^{2a}, R^{2b}, R^{2c}, R^{2d} and R^{2e} are independently —H, —Cl, —CH₃, OH—, —O—CH₃ or OH—CH₂—CH₂—O—,

R^{3a}, R^{3b}, R^{3c} and R^{3d} are independently —H or —F;

R⁴ is —H, —Br and —Cl;

R⁵ is —H or absent when Z is N;

R⁶ is —CH₃ or CH₃—S—; and

R^{7a}, R^{7b}, R^{7c}, R^{7d} and R^{7e} are independently —H, —C(O)—O—CH₃, —CH₃, —C(O)—OH,

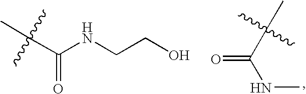

### (OH)₂—(CH₂)₃—NH—C(O)—,

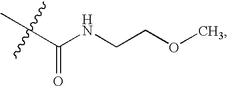

### NH₂—C(O)—CH₂—NH₂—C(O)—,

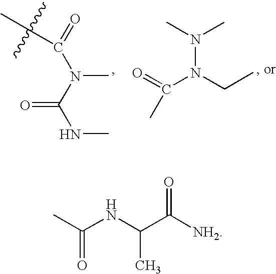

In one embodiment, the present invention provides a compound of Formula VI:

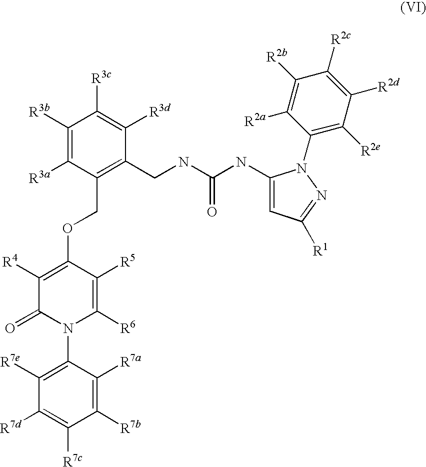

wherein;
R¹ is (C₁-C₄)-alkyl;
R^{2a}, R^{2b}, R^{2c}, R^{2d} and R^{2e} are independently H, (C₁-C₄)-alkyl, OH—, -halo, (C₁-C₄)-alkyl-O or OH—(C₁-C₄)-alkyl-O—;
R^{3a}, R^{3b}, R^{3c} and R^{3d} are independently —H or -halo;
R⁴ is —H or -halo;
R⁵ is —H;
R⁶ is —(C₁-C₄)-alkyl or (C₁-C₄)-alkyl-S—; and
R^{7a}, R^{7b}, R^{7c}, R^{7d} and R^{7e} are independently H, —C(O)—O—(C₁-C₄)-alkyl, (C₁-C₄)-alkyl, —C(O)—OH, OH—(C₁-C₄)-alkyl-NH—C(O)—, (C₁-C₄)-alkyl-NH—C(O)—, (OH)₂—(C₁-C₄)-alkyl-NH—C(O)—, (C₁-C₄)-alkyl-O—(C₁-C₄)-alkyl-NH—C(O)—, NH₂—C(O)—(C₁-C₄)-alkyl-NH—C(O)—, (C₁-C₄)-alkyl-NH—C(O)—(C₁-C₄)-alkyl-NH—C(O)—, (C₁-C₄)-dialkyl-N—(C₁-C₄)-alkyl-NH—C(O)— or [NH₂—C(O)][(C₁-C₄)-alkyl](C₁-C₄)-alkyl-NH—C(O)—.

In another embodiment, the present invention provides a compound of Formula VI:

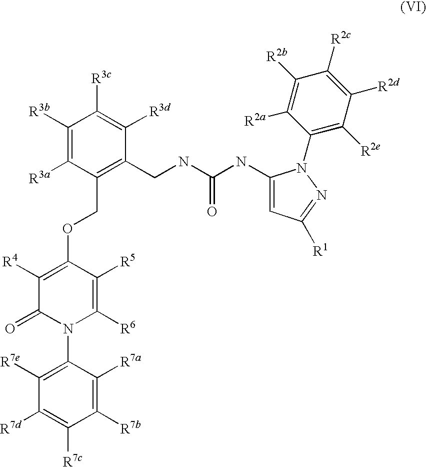

wherein;
R¹ is —C—(CH₃)₃;
R^{2a}, R^{2b}, R^{2c}, R^{2d} and R^{2e} are independently —H, —Cl, —CH₃, OH—, —CH₃ or OH—CH₂—CH₂—O—,
R^{3a}, R^{3b}, R^{3c} and R^{3d} are independently —H or —F;
R⁴ is —H, —Br or —Cl;
R⁵ is —H;
R⁶ is —CH₃ or CH₃—S—; and
R^{7a}, R^{7b}, R^{7c}, R^{7d} and R^{7e} are independently —H, —C(O)—O—CH₃, —CH₃, —C(O)—OH,

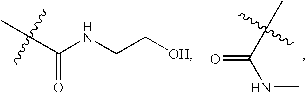

### (OH)₂—(CH₂)₃—NH—C(O)—, NH₂—C(O)—CH₂—NH₂—C(O)—,

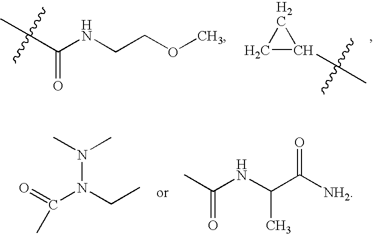

In one embodiment, the present invention provides a compound of Formula VII:

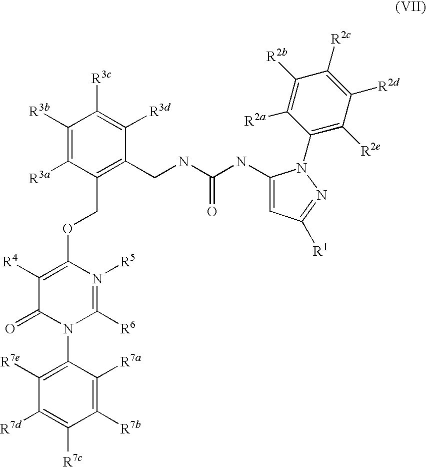

wherein;
R¹ is (C₁-C₄)-alkyl;
R^{2a}, R^{2b}, R^{2c}, R^{2d} and R^{2e} are independently —H or (C₁-C₄)-alkyl;
R^{3a}, R^{3b}, R^{3c} and R^{3d} are independently —H or halo;
R⁴ is —H or halo;
R⁵ is —H;
R⁶ is (C₁-C₄)-alkyl-S—; and
R^{7a}, R^{7b}, R^{7c}, R^{7d} and R^{7e} are independently —H, —C(O)—O—(C₁-C₄)-alkyl or (C₁-C₄)-alkyl.

In another embodiment, the present invention provides a compound of Formula VII:

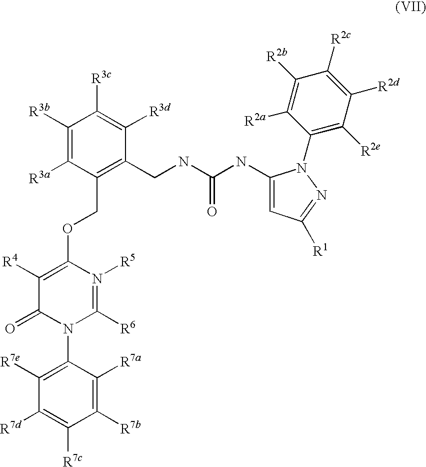

wherein;
R¹ is —C—(CH₃)₃;
R^{2a}, R^{2b}, R^{2c}, R^{2d} and R^{2e} are independently H or —CH₃;
R^{3a}, R^{3b}, R^{3c} and R^{3d} are independently H or F;
R⁴ is H or Br;
R⁵ is H;
R⁶ is CH₃—S—; and
R^{7a}, R^{7b}, R^{7c}, R^{7d} and R^{7e} are independently H, —C(O)—O—CH₃ or —CH₃.

In one embodiment, the present invention provides a compound of Formula I:

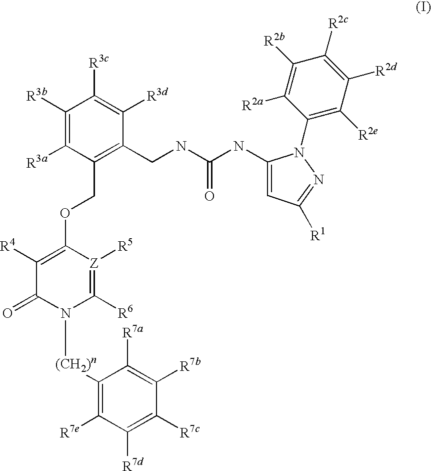

wherein;
Z is C or N;
n is 1;
R¹ is (C₁-C₄)-alkyl, (C₃-C₆)-cycloalkyl, or [(C₁-C₄)-alkyl)][(C₁-C₄)-alkyl-S]—(C₁-C₄)-alkyl;
R^{2a}, R^{2b}, R^{2c}, R^{2d} and R^{2e} are independently —H, (C₁-C₄)-alkyl, OH—, halo, (C₁-C₄)-alkyl-O—, heterocyclyloxy-(C₁-C₄)-alkyl-O—, OH—(C₁-C₄)-alkyl-O—, or (C₁-C₄)-alkyl[(C₁-C₄)-dialkyl]silyloxy;
R^{3a}, R^{3b}, R^{3c} and R^{3d} are independently —H or -halo;
R⁴ is —H, -halo or —(C₁-C₄)-alkyl;
R⁵ is —H or absent when Z is N;
R⁶ is —H or —(C₁-C₄)-alkyl; and
R^{7a}, R^{7b}, R^{7c}, R^{7d} and R^{7e} are independently —H, —C(O)—O—(C₁-C₄)-alkyl, (C₁-C₄)-alkyl-S—, (C₁-C₄)-alkyl-O—, OH—, NH₂—(C₁-C₄)-alkyl-C(O)—NH—(C₁-C₄)-alkyl, (C₁-C₄)-alkyl-O—C(O)—NH—(C₁-C₄)-alkyl-C(O)—NH—(C₁-C₄)-alkyl, halo,

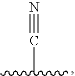

or OH—(C₁-C₄)-alkyl-C(O)—NH—(C₁-C₄)-alkyl.

In one embodiment, the present invention provides a compound of Formula I:

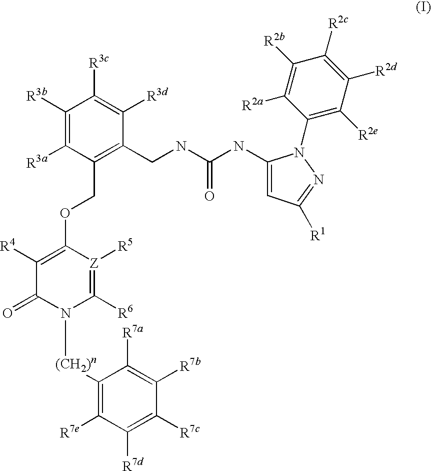

wherein;

Z is C;

n is 1;

R¹ is (C₁-C₄)-alkyl, (C₃-C₆)-cycloalkyl, or [(C₁-C₄)-alkyl)][(C₁-C₄)-alkyl-S]—(C₁-C₄)-alkyl;

R^{2a}, R^{2b}, R^{2c}, R^{2d} and R^{2e} are independently H, (C₁-C₄)-alkyl, OH—, halo, (C₁-C₄)-alkyl-O—, heterocyclyloxy-(C₁-C₄)-alkyl-O—, OH—(C₁-C₄)-alkyl-O—, or (C₁-C₄)-alkyl[(C₁-C₄)-dialkyl]silyloxy;

R^{3a}, R^{3b}, R^{3c} and R^{3d} are independently —H or -halo;

R⁴ is —H, -halo or —(C₁-C₄)-alkyl;

R⁵ is —H;

R⁶ is —(C₁-C₄)-alkyl; and

R^{7a}, R^{7b}, R^{7c}, R^{7d} and R^{7e} are independently —H, —C(O)—O—(C₁-C₄)-alkyl, (C₁-C₄)-alkyl-S—, (C₁-C₄)-alkyl-O—, OH—,
NH₂—(C₁-C₄)-alkyl-C(O)—NH—(C₁-C₄)-alkyl-, (C₁-C₄)-alkyl-O—C(O)—NH—, —(C₁-C₄)-alkyl-C(O)—NH—(C₁-C₄)-alkyl, halo,

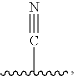

or OH—(C₁-C₄)-alkyl-C(O)—NH—(C₁-C₄)-alkyl.

In one embodiment, the present invention provides a compound of Formula I:

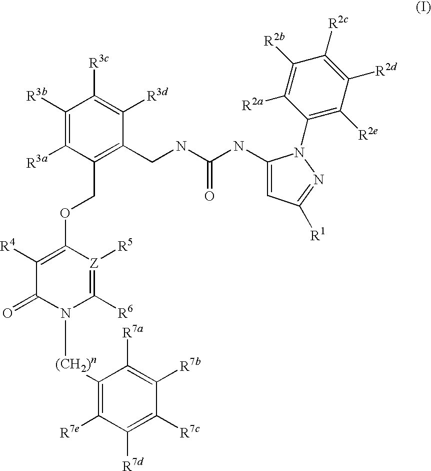

wherein;

Z is —N;

n is 1;

R¹ is —(C₁-C₄)-alkyl;

R^{2a}, R^{2b}, R^{2c}, R^{2d} and R^{2e} are independently —H, —(C₁-C₄)-alkyl, -halo or —(C₁-C₄)-alkyl-O—;

R^{3a}, R^{3b}, R^{3c} and R^{3d} are —H;

R⁴ is —(C₁-C₄)-alkyl;

R⁵ is absent;

R⁶ is —H; and

R^{7a}, R^{7b}, R^{7c}, R^{7d} and R^{7e} are independently H or (C₁-C₄)-alkyl-O—.

In one embodiment, the present invention provides a compound of Formula I:

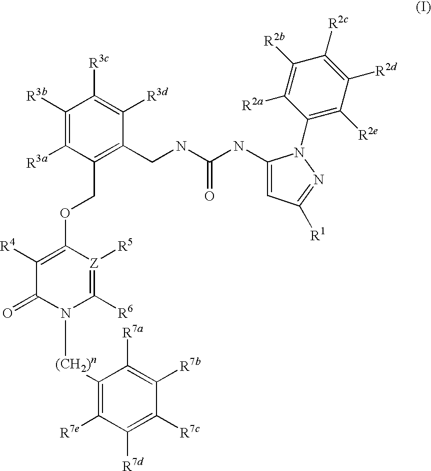

wherein;

Z is C or N;

n is 0;

R¹ is (C₁-C₄)-alkyl;

R^{2a}, R^{2b}, R^{2c}, R^{2d} and R^{2e} are independently —H, —(C₁-C₄)-alkyl, OH—, halo, (C₁-C₄)-alkyl-O— or OH—(C₁-C₄)-alkyl-O;

R^{3a}, R^{3b}, R^{3c} and R^{3d} are independently —H or -halo;

R⁴ is —H or -halo;

R⁵ is —H or absent when Z is N;

R⁶ is (C₁-C₄)-alkyl or (C₁-C₄)-alkyl-S—; and

R^{7a}, R^{7b}, R^{7c}, R^{7d} and R^{7e} are independently —H, —C(O)—O—(C₁-C₄)-alkyl, (C₁-C₄)-alkyl, —C(O)—OH, OH—(C₁-C₄)-alkyl-NH—C(O)—, (C₁-C₄)-alkyl-NH—C(O)—, (OH)₂—(C₁-C₄)-alkyl-NH—C(O)—, (C₁-C₄)-alkyl-O—(C₁-C₄)-alkyl-NH—C(O)—, NH₂—C(O)—(C₁-C₄)-alkyl-NH—C(O)—, (C₁-C₄)-alkyl-NH—C(O)—(C₁-C₄)-alkyl-NH—C(O)—, (C₁-C₄)-dialkyl-N—(C₁-C₄)-alkyl-NH—C(O)—, or [NH—C(O)]-[(C₁-C₄)-alkyl](C₁-C₄)-alkyl-NH—C(O)—.

In one embodiment, the present invention provides a compound of Formula I:

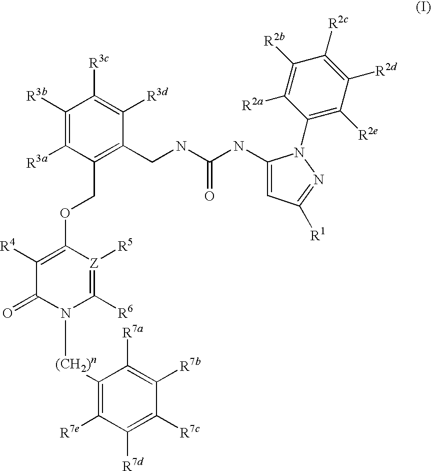

wherein;

Z is C;

n is 0;

R¹ is (C₁-C₄)-alkyl;

R^{2a}, R^{2b}, R^{2c}, R^{2d} and R^{2e} are independently H, (C₁-C₄)-alkyl, OH—, halo, (C₁-C₄)-alkyl-O— or OH—(C₁-C₄)-alkyl-O—;

R^{3a}, R^{3b}, R^{3c} and R^{3d} are independently —H or -halo;

R⁴ is —H or -halo;

R⁵ is —H;

R⁶ is —(C₁-C₄)-alkyl or (C₁-C₄)-alkyl-S—; and

R^{7a}, R^{7b}, R^{7c}, R^{7d} and R^{7e} are independently H, —C(O)—O—(C₁-C₄)-alkyl, (C₁-C₄)-alkyl, —C(O)—OH, OH—(C₁-C₄)-alkyl-NH—C(O)—, (C₁-C₄)-alkyl-NH—C(O)—, (OH)₂—(C₁-C₄)-alkyl-NH—C(O)—,
(C₁-C₄)-alkyl-O—(C₁-C₄)-alkyl-NH—C(O)—, NH₂—C(O)—(C₁-C₄)-alkyl-NH—C(O)—,
(C₁-C₄)-alkyl-NH—C(O)—(C₁-C₄)-alkyl-NH—C(O)—,
(C₁-C₄)-dialkyl-N—(C₁-C₄)-alkyl-NH—C(O)—, or [NH₂—C(O)][(C₁-C₄)-alkyl](C₁-C₄)-alkyl-NH—C(O)—.

In one embodiment, the present invention provides a compound of Formula I:

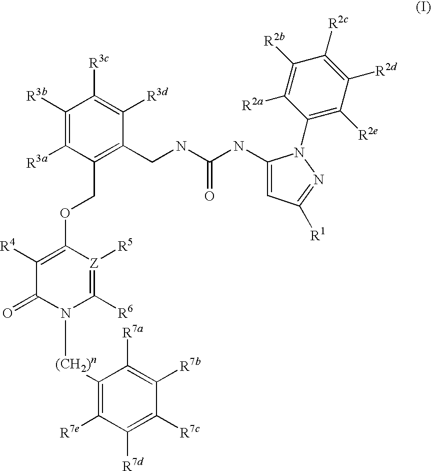

wherein;

Z is —N;

n is 0;

R¹ is (C₁-C₄)-alkyl;

R^{2a}, R^{2b}, R^{2c}, R^{2d} and R^{2e} are independently —H or (C₁-C₄)-alkyl;

R^{3a}, R^{3b}, R^{3c} and R^{3d} are independently —H or halo;

R⁴ is —H or halo;

R⁵ is —H or absent when Z is N;

R⁶ is (C₁-C₄)-alkyl-S—; and

R^{7a}, R^{7b}, R^{7c}, R^{7d} and R^{7e} are independently —H, —C(O)—O—(C₁-C₄)-alkyl or (C₁-C₄)-alkyl.

In one embodiment, the present invention provides a compound of Formula I:

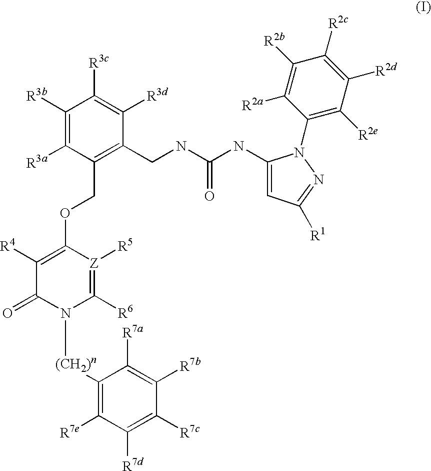

wherein;

Z is C or N;

n is 1;

R¹ is —C—(CH₃)₃,

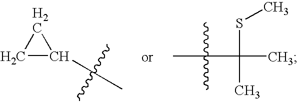

R^{2a}, R^{2b}, R^{2c}, R^{2d} and R^{e} are independently —H, —Cl, —CH₃, OH—, —O—CH₃, OH—CH₂—CH₂—OH—CH₂—CH₂—O—, —F, difluoropyranyloxyethoxy or tert-butyl[dimethyl]silyloxy;

R^{3a}, R^{3b}, R^{3c} and R^{3d} are independently —H or —F;

R⁴ is —H, —Br, —Cl, —CH₃ or —CH₂—CH₃;

R⁵ is H or absent when Z is N;

R⁶ is H or —CH₃; and

R^{7a}, R^{7b}, R^{7c}, R^{7d} and R^{7e} are independently —H, —C(O)—O—CH₃, CH₃—S—, —O—CH₃, OH—, NH₂—CH₂—C(O)—NH—CH₂—,

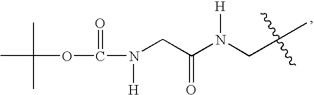

### Cl,

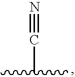

### or OH—CH2-C(O)—NH—CH2-.

In one embodiment, the present invention provides a compound of Formula I:

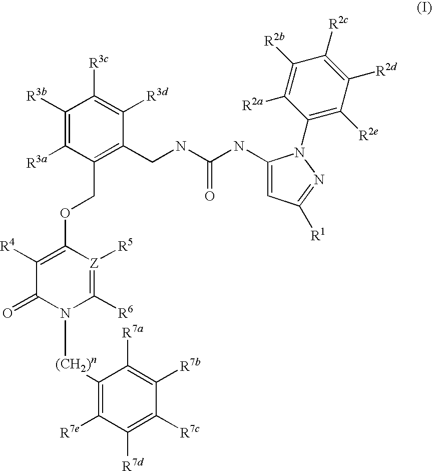

wherein;

Z is C;

n is 1;

R¹ is —C—(CH₃)₃,

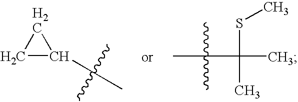

R^{2a}, R^{2b}, R^{2c}, R^{2d} and R^{2e} are independently —H, —Cl, —CH₃, —OH—, —O—CH₃, OH—CH₂—CH₂—O—, —F, difluoropyranyloxyethoxy or

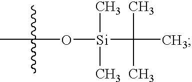

R^{3a}, R^{3b}, R^{3c} and R^{3d} are independently —H or —F;

R⁴ is —H, —Br, —Cl or —CH₃;

R⁵ is —H;

R⁶ is —CH₃; and

R^{7a}, R^{7b}, R^{7c}, R^{7d} and R^{7e} are independently —H, —C(O)—O—CH₃, CH₃—S—, —O—CH₃, OH—, NH₂—CH₂—C(O)—NH—CH₂—, OH—CH2-C(O)—NH—CH2-Cl.

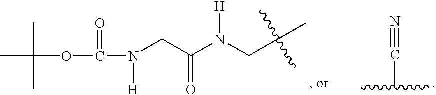

In one embodiment, the present invention provides a compound of Formula I:

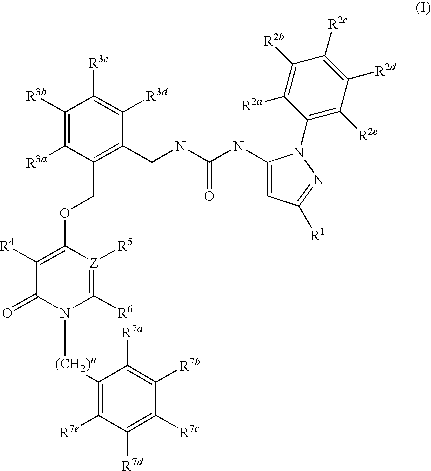

wherein;

Z is N;

n is 1;

R¹ is —C—(CH₃)₃;

R^{2a}, R^{2b}, R^{2c}, R^{2d} and R^{2e} are independently H, —CH₃, —F or —O—CH₃;

R^{3a}, R^{3b}, R^{3c} and R^{3d} are —H;

R⁴ is —CH₂—CH₃;

R⁵ is absent;

R⁶ is —H; and

R^{7a}, R^{7b}, R^{7c}, R^{7d} and R^{7e} are independently —H or —O—CH₃.

In one embodiment, the present invention provides a compound of Formula I:

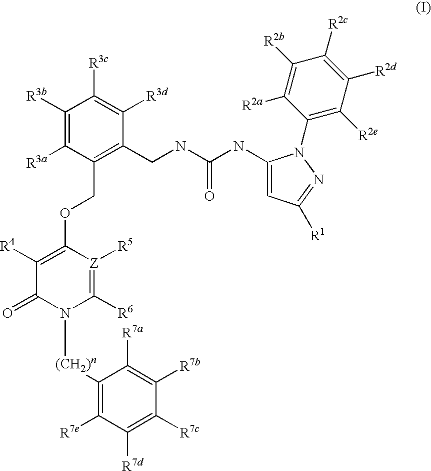

wherein;

Z is C or N;

n is 0;

R¹ is —C—(CH₃)₃;

R^{2a}, R^{2b}, R^{2c}, R^{2d} and R^{2e} are independently —H, —Cl, —CH₃, —OH—, —O—CH₃ or, OH—CH₂—CH₂—O—;

R^{3a}, R^{3b}, R^{3c} and R^{3d} are independently —H or —F;

R⁴ is —H, —Br or —Cl;

R⁵ is —H or absent when Z is —N;

R⁶ is —CH₃ or CH₃—S—; and

R^{7a}, R^{7b}, R^{7c}, R^{7d} and R^{7e} are independently H, —C(O)—O—CH₃, —CH₃, —C(O)—OH,

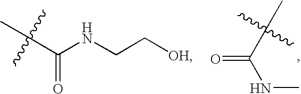

### (OH)₂—(CH₂)₃—NH—C(O)—,

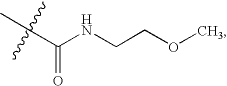

### NH₂—C(O)—CH₂—NH₂—C(O)—,

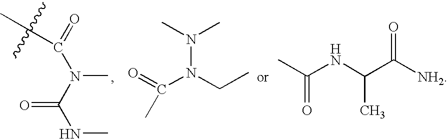

In one embodiment, the present invention provides a compound of Formula I:

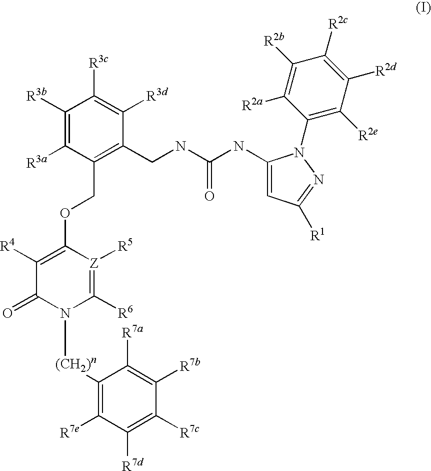

wherein;

Z is —C;

n is 0;

R¹ is —C—(CH₃)₃;

R^{2a}, R^{2b}, R^{2c}, R^{2d} and R^{2e} are independently —H, —Cl, —CH₃, OH—, —O—CH₃ or OH—CH₂—CH₂—O—,

R^{3a}, R^{3b}, R^{3c} and R^{3d} are independently —H or —F;

R⁴ is —H, —Br or —Cl;

R⁵ is —H;

R⁶ is —CH₃ or CH₃—S—; and

R^{7a}, R^{7b}, R^{7c}, R^{7d} and R^{7e} are independently —H, —C(O)—O—CH₃, —CH₃, —C(O)—OH,

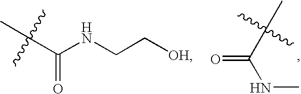

### (OH)₂—(CH₂)₃—NH—C(O)—,

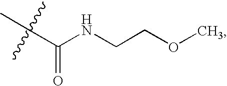

### NH₂—C(O)—CH₂—NH₂—C(O)—,

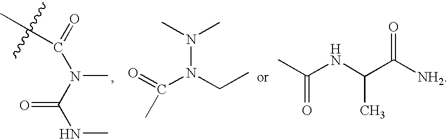

In one embodiment, the present invention provides a compound of Formula I:

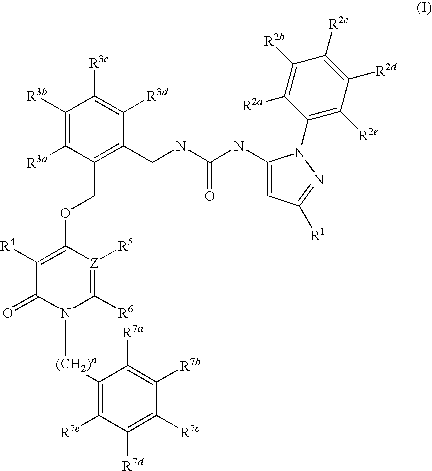

wherein;

Z is —N;

n is 0;

R¹ is —(CH₃)₃;

R^{2a}, R^{2b}, R^{2c}, R^{2d} and R^{2e} are independently —H or —CH₃;

R^{3a}, R^{3b}, R^{3c} and R^{3d} are independently —H or —F;

R⁴ is —H or —Br;

R⁵ is absent;

R⁶ is CH₃—S—; and

R^{7a}, R^{7b}, R^{7c}, R^{7d} and R^{7e} are independently H, —C(O)—O—CH₃ or —CH₃.

In one embodiment, the present invention provides a pharmaceutical composition comprising a compound of Formula I, II, III, IV, V, VI, or VII and a pharmaceutically acceptable excipient.

In one embodiment, the present invention provides a method for the treatment of an inflammatory disorder in a subject in need of such treatment, wherein the method comprises administering to the subject an amount of a compound of Formula I, II, III, IV, V, VI, or VII wherein the amount of the compound is effective for the treatment of an airways disease.

In one embodiment, the present invention provides a method for the treatment of an inflammatory disorder in a subject in need of such treatment wherein the method comprises administering to the subject an amount of a compound of Formula I, II, III, IV, V, VI, or VII wherein the amount of the compound is effective for the treatment of an inflammatory disorder.

In one embodiment, the inflammatory disorder is COPD.

In one embodiment, the inflammatory disorder is asthma.

In one embodiment, the inflammatory disorder is arthritis.

In one embodiment, the inflammatory disorder is osteoarthritis.

In one embodiment, the inflammatory disorder is rheumatoid arthritis.

This invention also is directed to tautomers of such compounds, as well as salts (particularly pharmaceutically-acceptable salts) of such compounds and tautomers.

This invention also is directed, in part, to a method for treating a disorder mediated by pathological p38 kinase activity (particularly p38α activity) in a mammal. The method comprises administering an above-described compound or pharmaceutically acceptable salt thereof, to the mammal in an amount that is therapeutically-effective to treat the disorder.

This invention also is directed, in part, to a method for treating a disorder mediated by pathological TNF activity (particularly TNF-α activity) in a mammal. The method comprises administering an above-described compound or pharmaceutically acceptable salt thereof, to the mammal in an amount that is therapeutically-effective to treat the disorder.

This invention also is directed, in part, to a method for treating a disorder mediated by pathological cyclooxygenase-2 activity in a mammal. The method comprises administering an above-described compound or pharmaceutically acceptable salt thereof, to the mammal in an amount that is therapeutically-effective to treat the disorder.

### Compounds of this Invention Having One or More Asymmetric Carbons

The present invention also comprises compounds of Formulas I, II, III, IV, V, VI, and VII having one or more asymmetric carbons. It is known to those skilled in the art that the compounds of the present invention having asymmetric carbon atoms may exist in diastereomeric, racemic, or optically active forms. All of these forms are contemplated within the scope of this invention. More specifically, the present invention includes enantiomers, diastereomers, racemic mixtures, and other mixtures thereof.

### Salts of the Compounds of this Invention

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound.

Pharmaceutically-acceptable acid addition salts of the compounds of this invention may be prepared from an inorganic or organic acid. Examples of suitable inorganic acids include hydrochloric, hydrobromic acid, hydroionic, nitric, carbonic, sulfuric, and phosphoric acid. Suitable organic acids generally include, for example, aliphatic, cycloaliphatic, aromatic, araliphatic, heterocyclyl, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, undecanoate and naphthalene-1,5-disulfonate.

Pharmaceutically-acceptable base addition salts of the compounds of this invention include, for example, metallic salts and organic salts.

In one embodiment of the present invention, metallic salts include alkali metal (group Ia) salts, alkaline earth metal (group IIa) salts, and other physiological acceptable metal salts. Such salts may be made from aluminum, calcium, lithium, magnesium, potassium, sodium, and zinc.

In another embodiment of the present invention, organic salts may be made from tertiary amines and quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

### Treating Disorders Using the Compounds of this Invention

This invention is directed, in part, to a method for treating a disorder (typically a pathological disorder) in mammals, such as humans, other primates (e.g., monkeys, chimpanzees. etc.), companion animals (e.g., dogs, cats, horses. etc.), farm animals (e.g., goats, sheep, pigs, cattle, etc.), laboratory animals (e.g., mice, rats, etc.), and wild and zoo animals (e.g., wolves, bears, deer, etc.) having or disposed to having such a disorder.

In this specification, the phrase “treating a disorder” means ameliorating, suppressing, eradicating, reducing the severity of, decreasing the frequency of incidence of preventing, reducing the risk of, or delaying the onset of the disorder.

Some embodiments of this invention are directed to a method for treating a p38-mediated disorder. As used herein, the term “p38-mediated disorder” refers to any disorder (particularly pathological disorders, i.e., diseases and disorders) in which p38 kinase (particularly p38α kinase) plays a role, either by control of p38 kinase itself, or by p38 kinase causing another factor to be released, such as, for example, IL-1, IL-6, or IL-8. A disease state in which, for instance, IL-1 is a major component, and whose production or action is exacerbated or secreted in response to p38, would therefore be considered a disorder mediated by p38.

The compounds of this invention generally are also useful for treating pathological disorders that include, but are not limited to:

asthma of whatever type, etiology, or pathogenesis, in particular asthma that is atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or in apparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis;
chronic or acute bronchoconstriction, chronic bronchitis, small airways obstruction, and emphysema;
obstructive or inflammatory airways diseases of whatever type, etiology, or pathogenesis, in particular an obstructive or inflammatory airways disease that is chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension;
bronchitis of whatever type, etiology, or pathogenesis, in particular bronchitis that is acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis;
acute lung injury; and
bronchiectasis of whatever type, etiology, or pathogenesis, in particular bronchiectasis that is cylindric bronchiectasis, sacculated bronchiectasis, fusiform bronchiectasis, capillary bronchiectasis, cystic bronchiectasis, dry bronchiectasis and follicular bronchiectasis.

The compounds of this invention generally are also useful in treating obstructive or inflammatory airways diseases of whatever type, etiology, or pathogenesis, in particular an obstructive or inflammatory airways disease that is chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension.

The compounds of this invention generally are useful for treating pathological disorders that include, but are not limited to:
(a) inflammation;
(b) arthritis, such as rheumatoid arthritis, spondyloarthropathies, gouty arthritis, osteoarthritis, systemic lupus erythematosus arthritis, juvenile arthritis, osteoarthritis, and gouty arthritis;
(c) neuroinflammation;
(d) pain (i.e., use of the compounds as analgesics), such as neuropathic pain;
(e) fever (i.e., use of the compounds as antipyretics);
(f) pulmonary disorders or lung inflammation, such as adult respiratory distress syndrome, pulmonary sarcoisosis, asthma, silicosis, and chronic pulmonary inflammatory disease;
(g) cardiovascular diseases, such as atherosclerosis, myocardial infarction (such as post-myocardial infarction indications), thrombosis, congestive heart failure, cardiac reperfusion injury, and complications associated with hypertension and/or heart failure such as vascular organ damage;
(h) cardiomyopathy;
(i) stroke, such as ischemic and hemorrhagic stroke;
(j) ischemia, such as brain ischemia and ischemia resulting from cardiac/coronary bypass;
(k) reperfusion injury;
(l) renal reperfusion injury;
(m) brain edema;
(n) neurotrauma and brain trauma, such as closed head injury;
(o) neurodegenerative disorders;
(p) central nervous system disorders (these include, for example, disorders having an inflammatory or apoptotic component), such as Alzheimer's disease, Parkinson's disease, Huntington's Disease, amyotrophic lateral sclerosis, spinal cord injury, and peripheral neuropathy;
(q) liver disease and nephritis;
(r) gastrointestinal disorders, such as inflammatory bowel disease, Crohn's disease, gastritis, irritable bowel syndrome, and ulcerative colitis;
(s) ulcerative diseases, such as gastric ulcer;
(t) ophthalmic diseases, such as retinitis, retinopathies (such as diabetic retinopathy), uveitis, ocular photophobia, nonglaucomatous optic nerve atrophy, and age-related macular degeneration (ARMD) (such as ARMD-atrophic form);
(u) opthalmological disorders, such as corneal graft rejection, ocular neovascularization, retinal neovascularization (such as neovascularization following injury or infection), and retrolental fibroplasia;
(v) glaucoma, such as primary open angle glaucoma (POAG), juvenile onset primary open-angle glaucoma, angle-closure glaucoma, pseudoexfoliative glaucoma, anterior ischemic optic neuropathy (AION), ocular hypertension, Reiger's syndrome, normal tension glaucoma, neovascular glaucoma, ocular inflammation, and corticosteroid-induced glaucoma;
(w) acute injury to the eye tissue and ocular traumas, such as post-traumatic glaucoma, traumatic optic neuropathy, and central retinal artery occlusion (CRAO);
(x) diabetes;
(y) diabetic nephropathy;
(z) skin-related disorders, such as psoriasis, eczema, burns, dermatitis, keloid formation, scar tissue formation, and angiogenic disorders;
(aa) viral and bacterial infections, such as sepsis, septic shock, gram negative sepsis, malaria, meningitis, opportunistic infections, cachexia secondary to infection or malignancy, cachexia secondary to acquired immune deficiency syndrome (AIDS), AIDS, ARC (AIDS related complex), pneumonia, and herpes virus;
(bb) myalgias due to infection;
(cc) influenza;
(dd) endotoxic shock;
(ee) toxic shock syndrome;
(ff) autoimmune disease, such as graft vs. host reaction and allograft rejections;
(gg) bone resorption diseases, such as osteoporosis;
(hh) multiple sclerosis;
(ii) disorders of the female reproductive system, such as endometriosis;
(jj) pathological, but non-malignant, disorders, such as hemaginomas (such as infantile hemaginomas), angiofibroma of the nasopharynx, and avascular necrosis of bone;
(kk) benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovarian cancer, cervical cancer, lung cancer, breast cancer, skin cancer such as squamus cell and basal cell cancers, prostate cancer, renal cell carcinoma, and other known cancers that affect epithelial cells throughout the body;
(ll) leukemia;
(mm) lymphoma, such as B cell lymphoma;
(nn) systemic lupus erthrematosis (SLE);
(oo) angiogenesis including neoplasia; and
(pp) metastasis.

Some embodiments of this invention are alternatively (or additionally) directed to a method for treating a TNF-mediated disorder. As used herein, the term “TNF-mediated disorder” refers to any disorder (particularly any pathological disorders, i.e., diseases or disorders) in which TNF plays a role, either by control of TNF itself, or by TNF causing another monokine to be released, such as, for example, IL-1, IL-6, and/or IL-8. A disease state in which, for instance, IL-1 is a major component and whose production or action is exacerbated or secreted in response to TNF, would therefore be considered a disorder mediated by TNF.

As TNF-β has close structural homology with TNF-α (also known as cachectin), and because each induces similar biologic responses and binds to the same cellular receptor, the synthesis of both TNF-α and TNF-β are inhibited by the compounds of this invention and thus are herein referred to collectively as “TNF” unless specifically delineated otherwise.

Some embodiments of this invention are alternatively (or additionally) directed to a method for treating a cyclooxygenase-2-mediated disorder. As used herein, the term “cyclooxygenase-2-mediated disorder” refers to any disorder (particularly pathological disorders, i.e., diseases and disorders) in which cyclooxygenase-2 plays a role, either by control of cyclooxygenase-2 itself, or by cyclooxygenase-2 causing another factor to be released. Many cyclooxygenase-2-mediated disorders are known in the art, and include, for example, inflammation and other cyclooxygenase-mediated disorders listed by Carter et al. in U.S. Pat. No. 6,271,253

According to another embodiment of the present invention, the compounds of the invention can also be used as a combination with one or more additional therapeutic agents to be co-administered to a patient to obtain some particularly desired therapeutic end result such as the treatment of pathophysiologically-relevant disease processes including, but not limited to (i) bronchoconstriction, (ii) inflammation, (iii) allergy, (iv) tissue destruction, (v) signs and symptoms such as breathlessness, cough. The second and more additional therapeutic agents may also be a compound of the invention, or one or more P38 and/or TNF inhibitors known in the art. More typically, the second and more therapeutic agents will be from a different class of therapeutic agents.

As used herein, the terms “co-administration”, “co-administered” and “in combination with”, referring to the compounds of the invention and one or more other therapeutic agents, is intended to mean, and does refer to and include the following:

(a) simultaneous administration of such combination of compound(s) of the invention) and therapeutic agent(s) to a patient in need of treatment, when such components are formulated together into a single dosage form which releases said components at substantially the same time to said patient,

(b) substantially simultaneous administration of such combination of compound(s) of the invention and therapeutic agent(s) to a patient in need of treatment, when such components are formulated apart from each other into separate dosage forms which are taken at substantially the same time by said patient, whereupon said components are released at substantially the same time to said patient,

(c) sequential administration of such combination compound(s) of the invention and therapeutic agent(s) to a patient in need of treatment, when such components are formulated apart from each other into separate dosage forms which are taken at consecutive times by said patient with a significant time interval between each administration, whereupon said components are released at substantially different times to said patient; and

(d) sequential administration of such combination of compound(s) of the invention and therapeutic agent(s) to a patient in need of treatment, when such components are formulated together into a single dosage form which releases said components in a controlled manner whereupon they are concurrently, consecutively, and/or overlappingly administered at the same and/or different times by said patient, where each part may be administered by either the same or different route.

Suitable examples of other therapeutic agents which may be used in combination with the compound(s) of the invention, or pharmaceutically acceptable salts, solvates or compositions thereof, include, but are by no means limited to:

5-Lipoxygenase (5-LO) inhibitors or 5-lipoxygenase activating protein (FLAP) antagonists,
Leukotriene antagonists (LTRAs) including antagonists of LTB₄, LTC₄, LTD₄, and LTE₄,
Histamine receptor antagonists including H1 and H3 antagonists,
α₁- and α₂-adrenoceptor agonist vasoconstrictor sympathomimetic agents for decongestant use,
muscarinic M3 receptor antagonists or anticholinergic agents,
PDE inhibitors, e.g. PDE3, PDE4 and PDE5 inhibitors,

### Theophylline,

Sodium cromoglycate,
COX inhibitors both non-selective and selective COX-1 or COX-2 inhibitors (NSAIDs),
Oral and inhaled glucocorticosteroids, such as DAGR (dissociated agonists of the corticoid receptor)
Monoclonal antibodies active against endogenous inflammatory entities, β2 agonists
Adhesion molecule inhibitors including VLA-4 antagonists,
Kinin-B₁- and B₂-receptor antagonists,
Immunosuppressive agents,
Inhibitors of matrix metalloproteases (MMPs),
Tachykinin NK₁, NK₂ and NK₃ receptor antagonists,
Elastase inhibitors,
Adenosine A2a receptor agonists,
Inhibitors of urokinase,
Compounds that act on dopamine receptors, e.g. D2 agonists,
Modulators of the NFκβ pathway, e.g. IKK inhibitors,
modulators of cytokine signalling pathways such as syk kinase, or JAK kinase inhibitors,
Agents that can be classed as mucolytics or anti-tussive,

### Antibiotics,

HDAC (histone deacetylase) inhibitors, and
PI3 kinase inhibitors.

According to one embodiment of the present invention, combination of the compounds of the invention with:
H3 antagonists,
Muscarinic M3 receptor antagonists,
PDE4 inhibitors,
glucocorticosteroids,
Adenosine A2a receptor agonists,
β2 agonists
Modulators of cytokine signalling pathways such as syk kinase, or,
Leukotriene antagonists (LTRAs) including antagonists of LTB₄, LTC₄, LTD₄, and LTE₄,

can be used.

According to one embodiment of the present invention, combination of the compounds of the invention with:

glucocorticosteroids, in particular inhaled glucocorticosteroids with reduced systemic side effects, including prednisone, prednisolone, flunisolide, triamcinolone acetonide, beclomethasone dipropionate, budesonide, fluticasone propionate, ciclesonide, and mometasone furoate,

muscarinic M3 receptor antagonists or anticholinergic agents including in particular ipratropium salts, namely bromide, tiotropium salts, namely bromide, oxitropium salts, namely bromide, perenzepine, and telenzepine,

or β2 agonists can be used.

A wide variety of methods may be used alone or in combination to administer the compounds described above. For example, the compounds may be administered orally, intravascularly (IV), intraperitoneally, subcutaneously, intramuscularly (IM), by inhalation spray, rectally, or topically.

Typically, a compound described in this specification is administered in an amount effective to inhibit p38 kinase (particularly p38α kinase), TNF (particularly TNF-α), and/or cyclooxygenase (particularly cyclooxygenase-2).

In one embodiment of the present invention, the total daily dose of the compound (administered in single or divided doses) is typically from about 0.01 to about 100 mg/kg. In another embodiment of the present invention, the total daily dose of the compound is typically from about 0.1 to about 50 mg/kg. In still another embodiment of the present invention, the total daily dose of the compound is from about 0.5 to about 30 mg/kg (i.e., mg compound per kg body weight). Dosage unit compositions may contain such amounts or submultiples thereof to make up the daily dose. In many instances, the administration of the compound will be repeated a plurality of times in a day (typically no greater than 4 times), Multiple doses per day typically may be used to increase the total daily dose, if desired.

Factors affecting the dosage regimen include the type, age, weight, sex, diet, and disorder of the patient; the severity of the pathological disorder; the route of administration; pharmacological considerations, such as the activity, efficacy, pharmacokinetic, and toxicology profiles of the particular compound employed; whether a drug delivery system is utilized; and whether the compound is administered as part of a drug combination. Thus, the dosage regimen actually employed can vary widely, and, therefore, can deviate from the dosage regimen set forth above.

The present compounds may be used in co-therapies, partially or completely, in place of other conventional anti-inflammatory, such as together with steroids, cyclooxygenase-2 inhibitors, non-steroidal anti-inflammatory drugs (“NSAIDs”), disease-modifying anti-rheumatic drugs (“DMARDs”), immunosuppressive agents, 5-lipoxygenase inhibitors, leukotriene B4 (“LTB4”) antagonists, and leukotriene A4 (“LTA4”) hydrolase inhibitors.

### Pharmaceutical Compositions Containing the Compounds of this Invention

This invention also is directed to pharmaceutical compositions (or “medicaments”) comprising the compounds described above (including tautomers of the compounds, and pharmaceutically-acceptable salts of the compounds and tautomers), and to methods for making pharmaceutical compositions comprising those compounds in combination with one or more conventional non-toxic, pharmaceutically-acceptable carriers, diluents, wetting or suspending agents, vehicles, and/or adjuvants (the carriers, diluents, wetting or suspending agents, vehicles, and adjuvants sometimes being collectively referred to in this specification as “carrier materials”); and/or other active ingredients. The composition depends on the method of administration. Formulation of drugs is generally discussed in, for example, Hoover, John E., Remington's Pharmaceutical Sciences (Mack Publishing Co., Easton, Pa.: 1975) (incorporated by reference into this specification). See also, Liberman, H. A., Lachman, L., eds., Pharmaceutical Dosage Forms (Marcel Decker, New York, N.Y., 1980) (incorporated by reference into this specification).

In many embodiments, the pharmaceutical composition is made in the form of a dosage unit containing a particular amount of the active ingredient. Typically, the pharmaceutical composition contains from about 0.1 to 1000 mg (and more typically, 7.0 to 350 mg) of the compound.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (in one embodiment of the invention, an atomiser with electrohydrodynamics can be utilized to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as 1-leucine, mannitol, or magnesium stearate. The lactose may be anhydrous or in the form of the monohydrate. In one embodiment the lactose is anhydrous. In another embodiment of the present invention, the lactose is in the form of the monohydrate. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.001 mg to 10 mg of the compound of the invention. The overall daily dose will typically be in the range 0.001 mg to 40 mg which may be administered in a single dose or, more usually, as divided doses throughout the day.

Solid dosage forms for oral administration include, for example, hard or soft capsules, tablets, pills, powders, and granules. In such solid dosage forms, the compounds are ordinarily combined with one or more adjuvants. If administered per os, the compounds may be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation, as may be provided in a dispersion of the compound of this invention in hydroxypropylmethyl cellulose. In the case of capsules, tablets, and pills, the dosage forms also may comprise buffering agents, such as sodium citrate, or magnesium or calcium carbonate or bicarbonate. Tablets and pills additionally may be prepared with enteric coatings.

Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

“Parenteral administration” includes subcutaneous injections, intravenous injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents. Acceptable carrier materials include, for example, water, 1,3-butanediol, Ringer's solution, isotonic sodium chloride solution, bland fixed oils (e.g., synthetic mono- or diglycerides), dextrose, mannitol, fatty acids (e.g., oleic acid), dimethyl acetamide, surfactants (e.g., ionic and non-ionic detergents), and/or polyethylene glycols (e.g., PEG 400).

Formulations for parenteral administration may, for example, be prepared from sterile powders or granules having one or more of the carriers materials mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

In one embodiment, the compounds of the present invention make up from about 0.075 to about 30% (w/w). In another embodiment, the compounds of the present invention make up from about 0.2 to 20% (w/w). In yet another embodiment of the present invention, the compounds make up from about 0.4 to 15% (w/w) of a pharmaceutical composition used for topical or rectal administration.

Suppositories for rectal administration may be prepared by, for example, mixing a compound of this invention with a suitable nonirritating excipient that is solid at ordinary temperatures, but liquid at the rectal temperature and will therefore melt in the rectum to release the drug. Suitable excipients include, for example, such as cocoa butter; synthetic mono-, di-, or triglycerides; fatty acids; and/or polyethylene glycols.

“Topical administration” includes transdermal administration, such as via transdermal patches or iontophoresis devices. Compositions for topical administration also include, for example, topical gels, sprays, ointments, and creams.

When formulated in an ointment, the compounds of this invention may be employed with, for example, either a paraffinic or a water-miscible ointment base. When formulated in a cream, the active ingredient(s) may be formulated with, for example, an oil-in-water cream base. If desired, the aqueous phase of the cream base may include, for example at least about 30% (w/w) of a polyhydric alcohol, such as propylene glycol, butane-1,3-diol, mannitol, sorbitol, glycerol, polyethylene glycol, and mixtures thereof.

A topical formulation may include a compound which enhances absorption or penetration of the active ingredient through the skin or other affected areas. Examples of such dermal penetration enhancers include dimethylsulfoxide and related analogs.

When the compounds of this invention are administered by a transdermal device, administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise, for example, a mixture of at least one emulsifier with a fat or an oil or with both a fat and an oil.

In one embodiment of the present invention, a hydrophilic emulsifier is included together with a lipophilic emulsifier which acts as a stabilizer. In another embodiment of the present invention, both an oil and a fat are included. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, given that the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters, for example, may be used. These may be used alone or in combination depending on the properties required. Alternatively, high melting point lipids such as white soft paraffin and/or liquid paraffin or other mineral oils may be used. Formulations suitable for topical administration to the eye also include eye drops wherein the compound of this invention is dissolved or suspended in suitable carrier, typically comprising an aqueous solvent.

In one embodiment of the present invention, the compounds of this invention are present in such formulations in a concentration of from about 0.5 to about 20% (w/w). In another embodiment of the present invention, the compounds are present in such formulations in a concentration of from about 0.5 to 10% (w/w). In yet another embodiment of the present invention, the compounds are present in such formulations in a concentration of from about 1.5% (w/w)).

Other carrier materials and modes of administration known in the pharmaceutical art may also be used.

### General Synthetic Procedures

Representative procedures for the preparation of compounds of the invention are outlined below in the Schemes. The starting materials can be purchased or prepared using methods known to those skilled in the art. Similarly, the preparation of the various intermediates can be achieved using methods known in the art. The starting materials may be varied and additional steps employed to produce compounds encompassed by the invention, as demonstrated by the examples below. In addition, different solvents and reagents can typically be used to achieve the above transformations. Furthermore, in certain situations, it may be advantageous to alter the order in which the reactions are performed. Protection of reactive groups may also be necessary to achieve the above transformations. In general, the need for protecting groups, as well as the disorders necessary to attach and remove such groups, will be apparent to those skilled in the art of organic synthesis. When a protecting group is employed, deprotection will generally be required. Suitable protecting groups and methodology for protection and deprotection such as those described in Protecting Groups in Organic Synthesis by Greene and Wuts are known and appreciated in the art.

The following schemes are representative of the methods that can be used to prepare these compounds.

An appropriately substituted pyranone is condensed with benzyl or phenyl amine. The resulting pyridinone is alkylated with a substituted benzyl halide to afford the benzyloxy pyridinone. This pyridinone can be manipulated via standard functional group interconversion or deprotection to afford the benzyl amine derivative. This benzyl amine derivative can be acylated and further converted to substituted ureas.

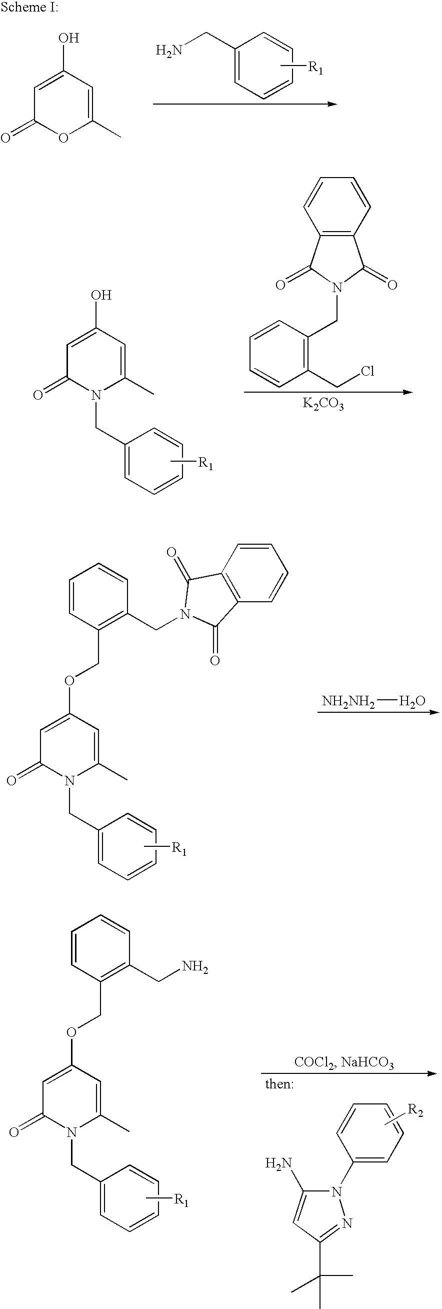

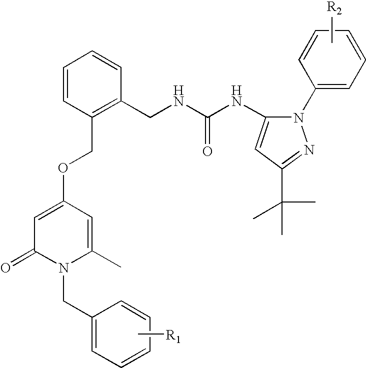

Alternatively the initially formed pyridinone can be halogenated to afford the halopyridinone. As in Scheme I this material can then be further elaborated.

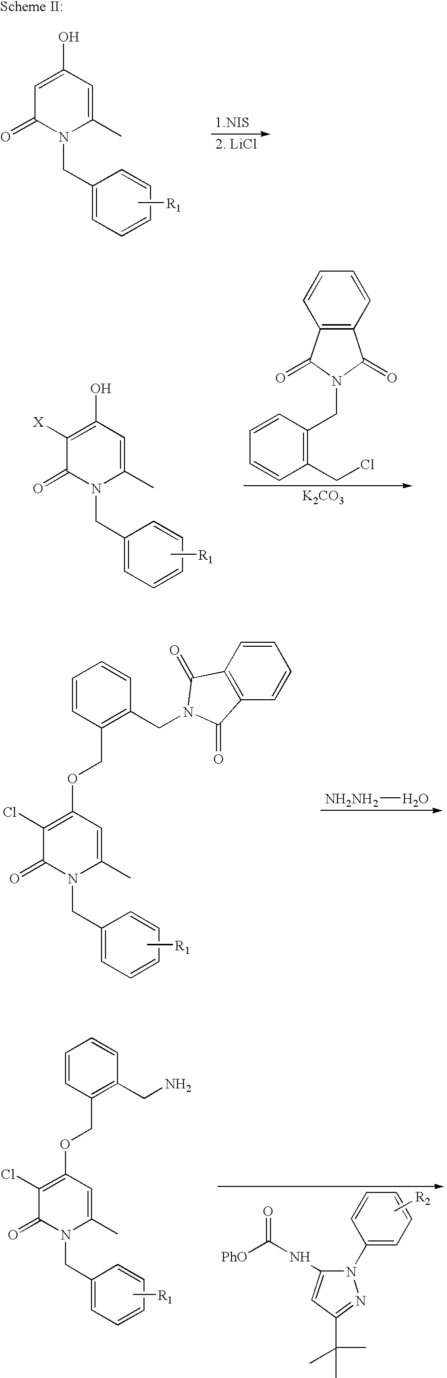

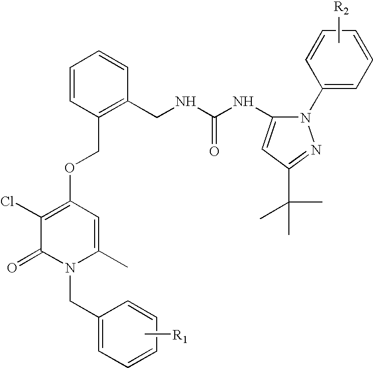

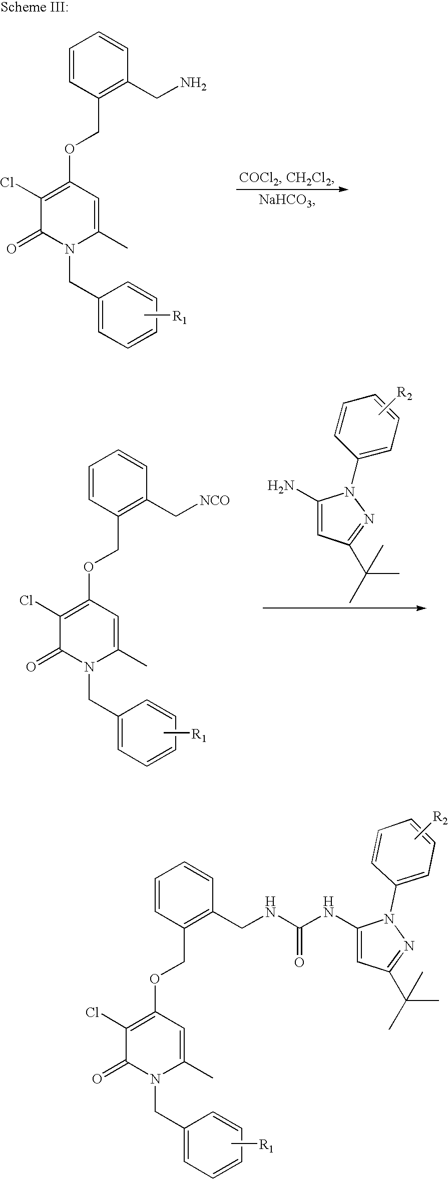

Generation of the iodopyridinone allows for the installation of alkyl groups via standard metal catalyzed reactions with suitably activated alkyl derivatives.

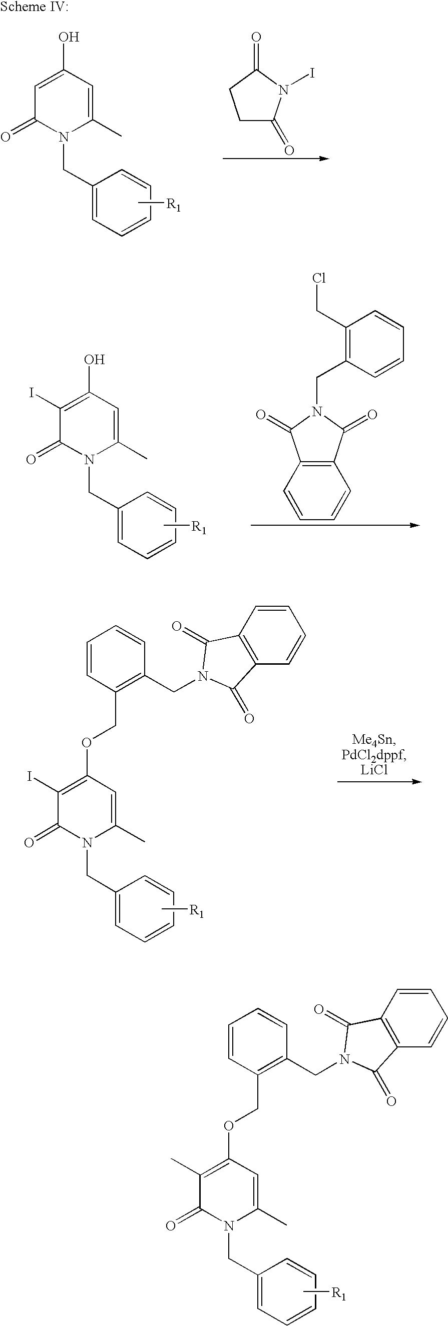

Condensation of phenyl carbamates of substituted amino pyrazoles allows for the generation of highly functionalized ureas.

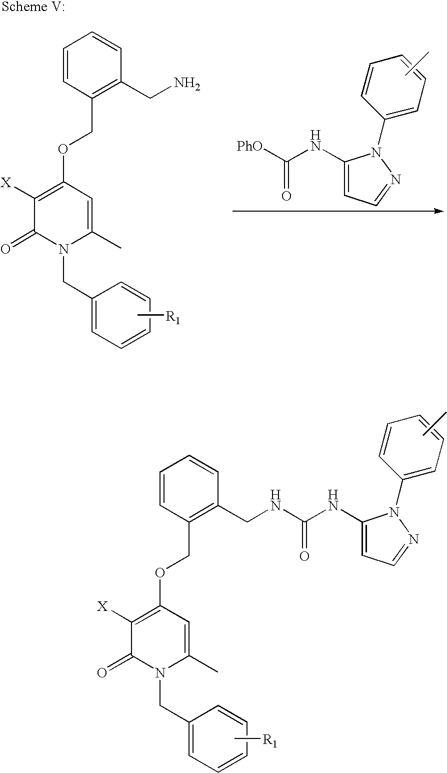

Halogenation of substituted benzyl amines affords the desired halobenzyl amines.

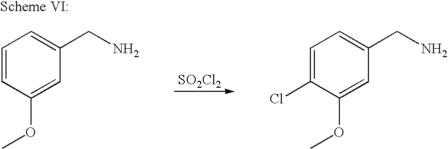

Deprotection of the N-dimethoxylbenzyl pyridinone affords the des-N-benzyl pyridinone. Alkylation of this pyridinone allows for the preparation of functionalized N-benzyl pyridinones that can be further manipulated as shown above.

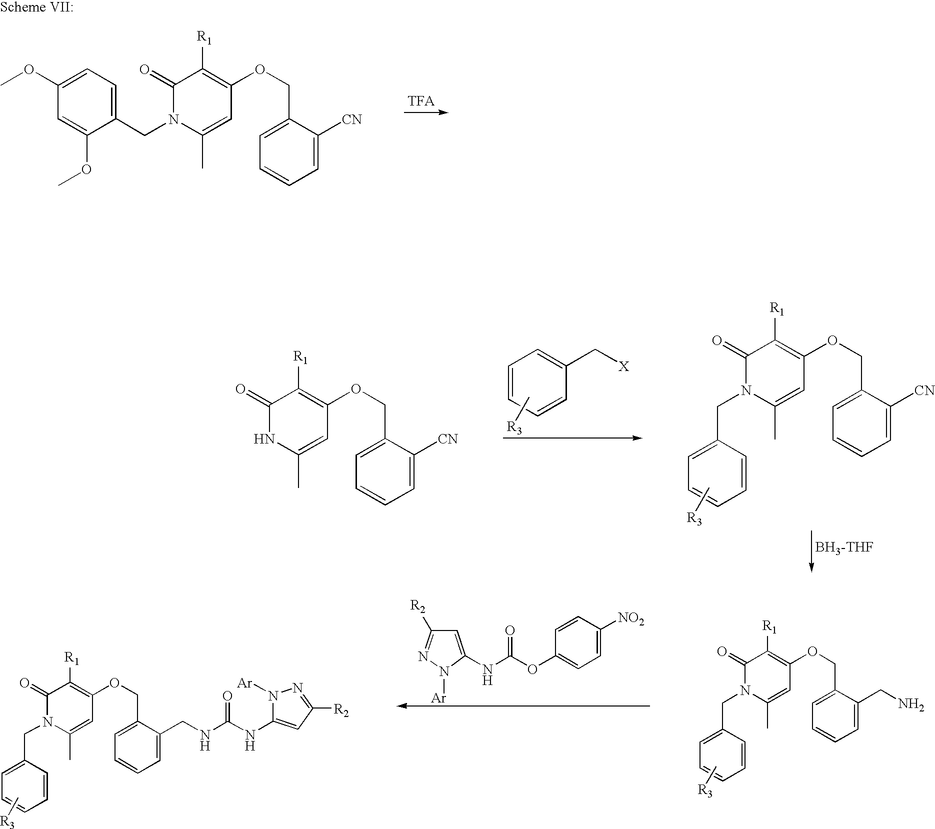

Alternatively the unsubstituted pyridinone can be O-benzylated to afford the NH-pyridinone. This intermediate can be N-benzylated and then further derivatized to the desired ureas.

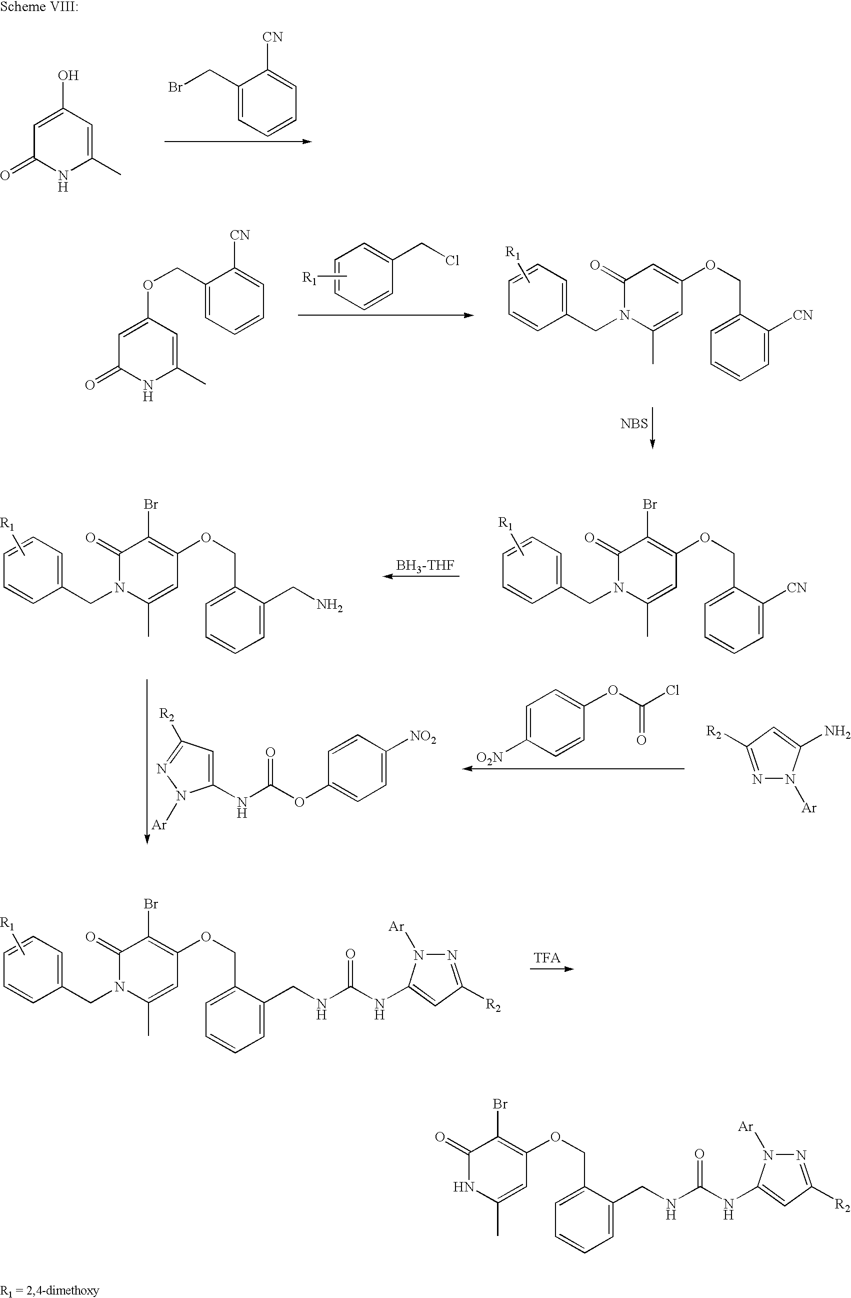

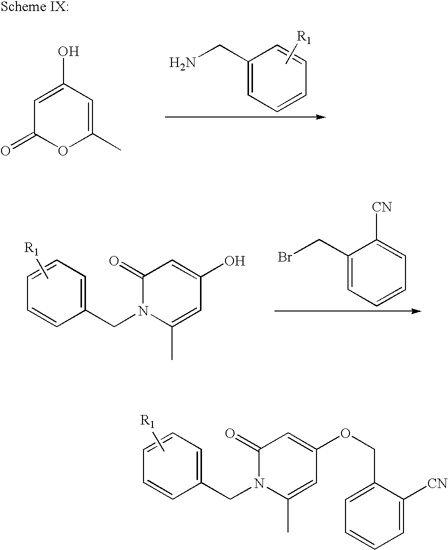

The corresponding pyrimidinones are prepared by mono-alkylation of the dihydroxy pyrimidinone derivative. The O-benzyl pyrimidinone is alkylated to afford the desired N-benzyl pyrimidinone. Manipulation of this material is analogous to the chemistry described above.

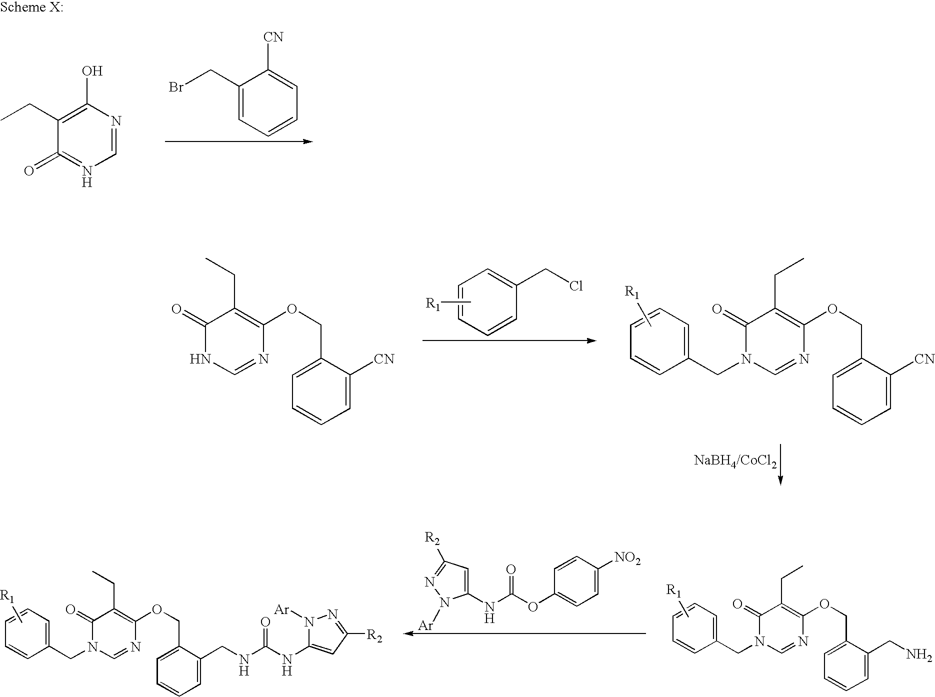

The elaborated pyrimidinone is iodinated and then converted to the methyl derivative via standard metal-catalyzed coupling reactions.

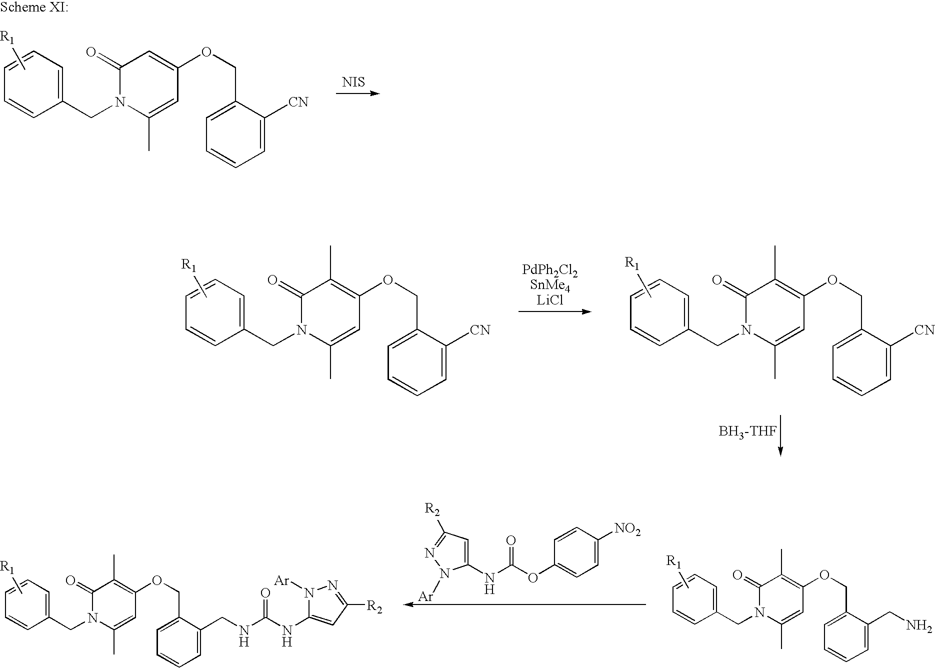

The N-phenyl pyridinones can be O-benzylated and further derivatized via conventional methods to afford the desired ureas.

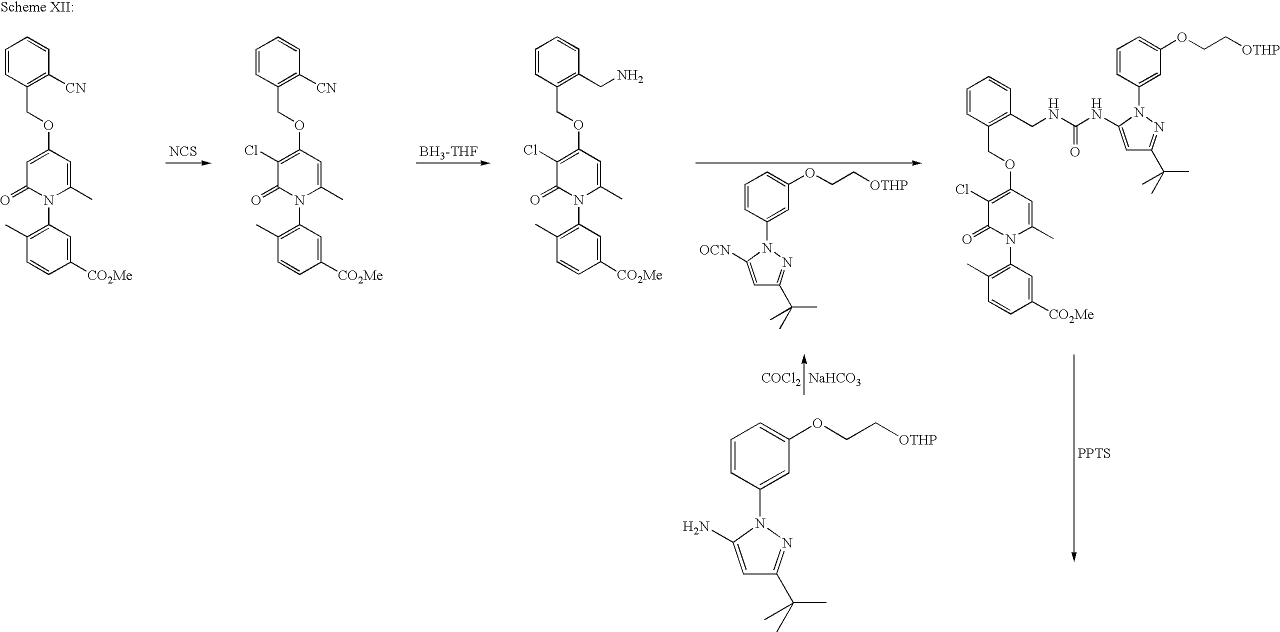

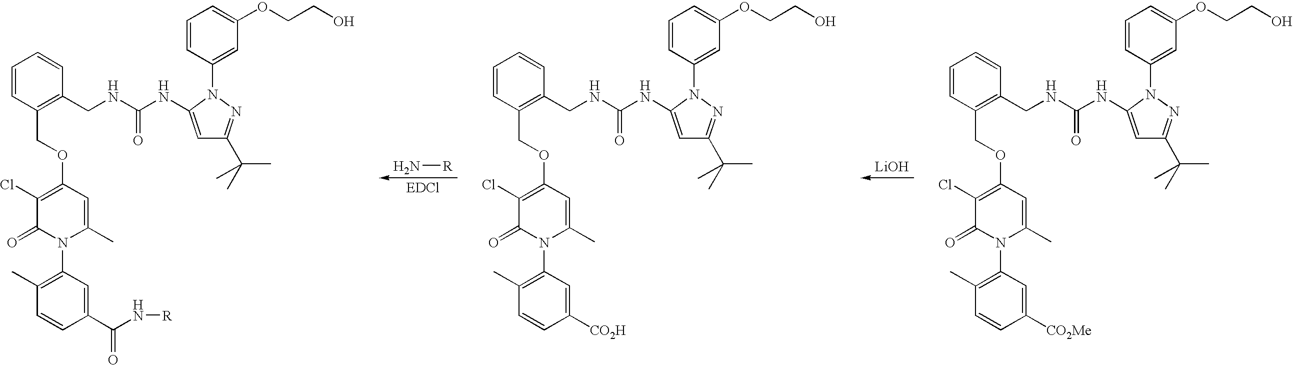

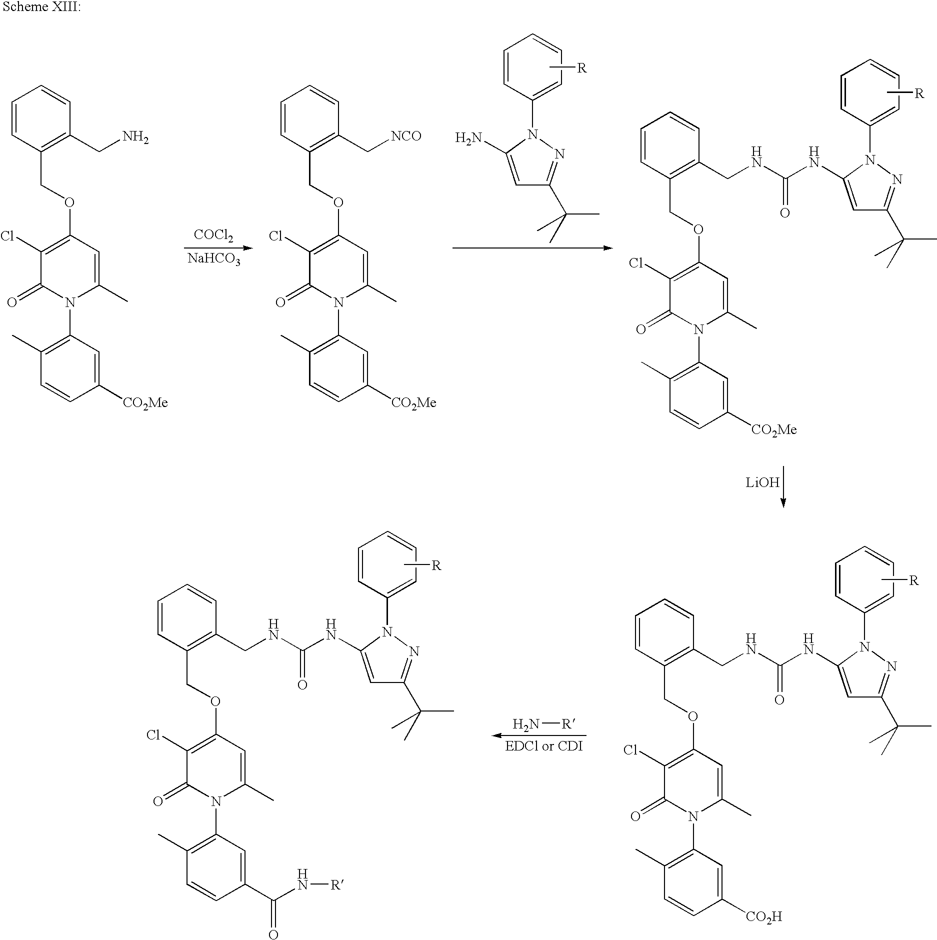

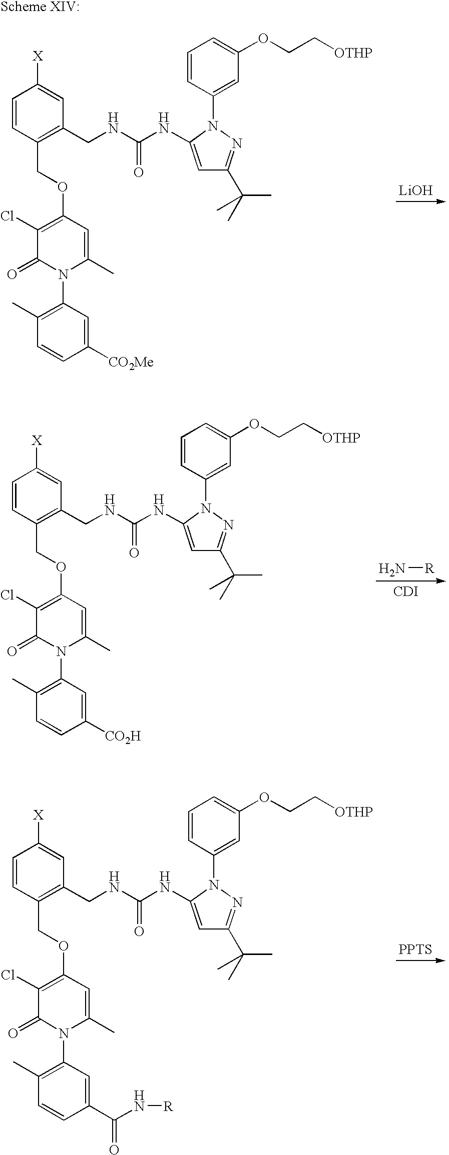

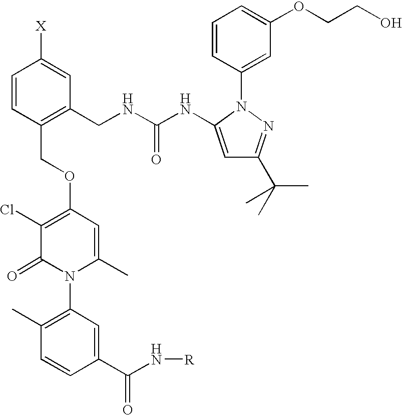

The N-phenyl pyrimidinones can be O-benzylated and further derivatized via conventional methods to afford the desired ureas.

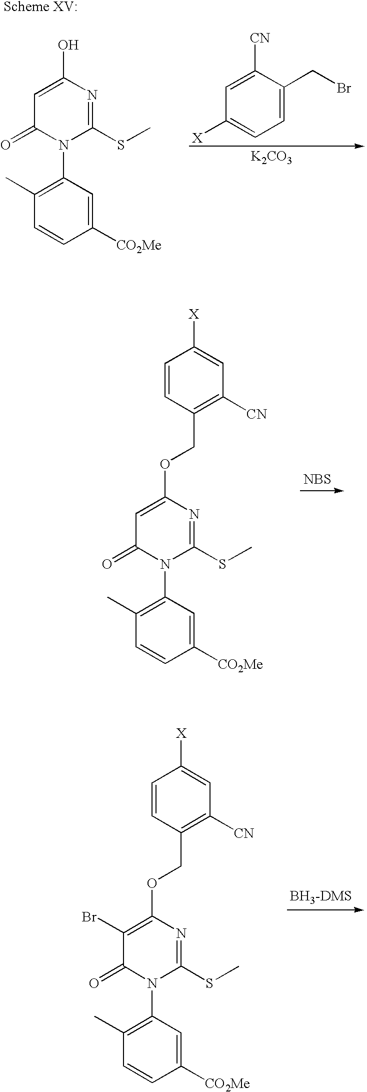

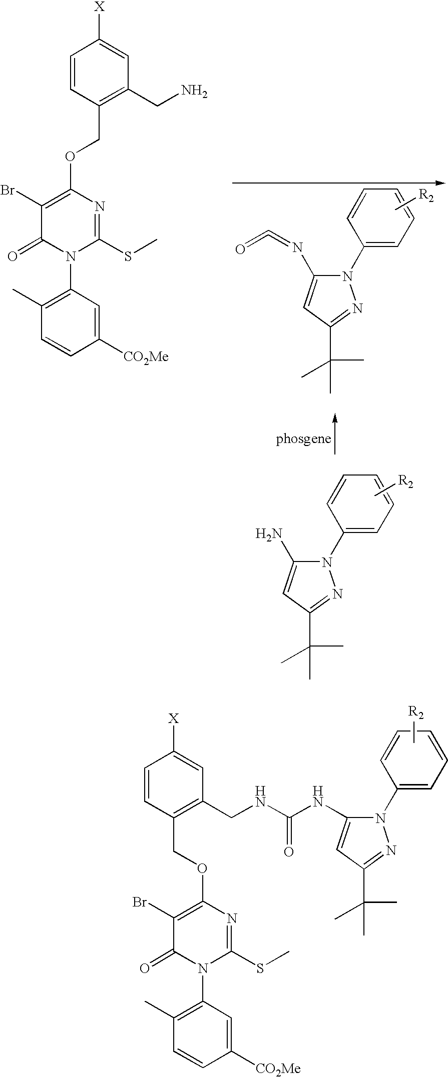

### Detailed Preparative Method

The detailed examples below illustrate preparation of compounds of this invention. Other compounds of this invention may be prepared using the methods illustrated in these examples, either alone or in combination with techniques generally known in the art. The following examples are merely illustrative, and not limiting to the remainder of this disclosure in any way.

The following abbreviations are used:

g—gram
mg—milligram
mmol—millimole
° C.—degrees celcius
M—molar
ml—milliliter
NMR—nuclear magnetic resonance
¹H—proton
MHz—megahertz
s—singlet
dd—doublet of doublets
d—doublet
t—triplet
q—quartet
br—broad
m—multiplet
app—apparent
J—coupling constant
Hz—hertz
LC/MS—liquid chromatograph/mass spectrometer
tᵣ—time of retention
min—minute
nm—nanometers
ES-MS—electrospray mass spectrometer
m/z—mass to charge ratio
ES-HRMS—electrospray high resolution mass spectrometer
calcd—calculated
N normal
L—liter
dq—doublet of quartets
dt—doublet of triplets
ddd—doublet of doublet of doublets
rt—room temperature
h—hour
ddt—doublet of doublet of triplets
w/w—weight to weight
psi—pounds per square inch
M+H—exact mass+1
HPLC—high performance liquid chromatography
DCM—dichloromethane
TFA—trifluoroacetic acid
DMF—dimethylformamide
DBU—1,8-Diazabicylo[5.4.0]-undec-7-ene

### NBS—N-Bromosuccinimide

### NCS—N-Chlorosuccinimide

ES-HRMS—Electrospray high-resolution mass spectrometry
t-BOC—tert-butyloxycarbonyl
DMAP—dimethylaminopyridine
DCM—dichloromethane
EtOAc—ethyl acetate
MCPBA—meta-Chloroperbenzoic acid

### EXAMPLES

### Preparation of Intermediates

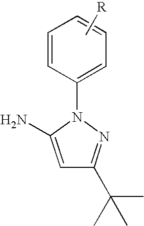

The following compounds (Intermediates 1i-8i) were prepared in a manner similar to that described in J. Med. Chem. 2002, 45 (14), 2994-3008.

| Intermediate | | | Molecular | |
|---|---|---|---|---|
| Number | R(t-Butyl pyrazoles) | Intermediate Name | Formula | HRMS |
| 1i | tolyl- | 3-tert-butyl-1-p-tolyl- | C14H19N3 | 230.17 |
| | | 1H-pyrazol-5-amine | | (M + H) |
| 2i | 3-methoxyphenyl- | 3-tert-butyl-1-(3- | C14H19N3O | 246.15 |
| | | methoxyphenyl)-1H- | | (M + H) |
| | | pyrazol-5-amine | | |
| 3i | (3-(2-(tetrahydro-2H- | 3-tert-butyl-1-(3-(2- | C20H29N3O3 | 360.02 |
| | pyran-2-yloxy)ethoxy)- | (tetrahydro-2H-pyran- | | (M + H) |
| | phenyl)- | 2-yloxy)ethoxy)phenyl)- | | |
| | | 1H-pyrazol-5-amine | | |
| 4i | 4-hydroxyphenyl | 4-(5-amino-3-tert- | C13H17N3O | 232.02 |
| | | butyl-1H-pyrazol-1- | | (M + H) |
| | | yl)phenol | | |
| 5i | 3-hydroxyphenyl | 3-(5-amino-3-tert- | C13H17N3O | 232.02 |
| | | butyl-1H-pyrazol-1- | | (M + H) |
| | | yl)phenol | | |
| 6i | 4-chloro-3- | 5-(5-amino-3-tert- | C13H16ClN3O | 266.04 |
| | hydroxyphenyl | butyl-1H-pyrazol-1- | | (M + H) |
| | | yl) 2-chlorophenol | | |
| 7i | 3-chloro-4- | 4-(5-amino-3-tert- | C13H16ClN3O | 266.14 |
| | hydroxyphenyl | butyl-1H-pyrazol-1- | | (M + H) |
| | | yl)-2-chlorophenol | | |
| 8i | 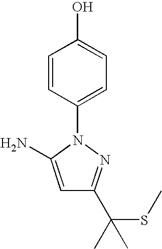 | 4-(5-amino-3-(2- (methylthio)propan-2- yl)-1H-pyrazol-1- yl)phenol | C13H17N3OS | 264.9 (M + H) |

Intermediate compounds 9i-26i were synthesized as described for each compound below as follows:

### Intermediate 9i

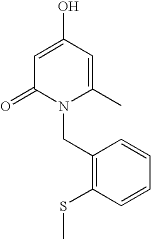

### 1-(2-(methylthio)benzyl)-4-hydroxy-6-methylpyridin-2(1H)-one

Under a nitrogen atmosphere 6-methyl-4-hydroxypyrone (43.4 g, 344 mmol) was dissolved in 510 mL water at 100° C., followed by the addition of 2-(methylthio)benzyl amine (11.11 g, 72.61 mmol) in roughly 0.9 g portions over 1.5 hours with stirring. After 17 hours the reaction was cooled to r.t., and the supernatant decanted off the gummy solid. The solid was then triturated in 75 mL acetone, filtered, and washed with acetone (2×25 mL). After drying under nitrogen, the solid was combined with 50 mL water and 50 mL of 1N aqueous sodium hydroxide, then sonicated for 30 min. The mixture was then filtered, and the residue washed with water (2×25 mL). The filtrate was then neutralized with 50 mL of 1N aqueous hydrochloric acid, filtered, and the product washed with water (3×25 mL), then dried in vacuo. This gave 11.465 g (58% yield) as a yellow solid. ¹H NMR (400 MHz, DMSO-d6) δ ppm 2.05 (s, 3H), 2.49 (s, 3H), 5.03 (s, 2H), 5.54 (d, J=2.4 Hz, 1H), 5.81 (d, J=2.1 Hz, 1H), 6.41 (d, J=7.5 Hz, 1H), 7.05 (t, J=7.5 Hz, 1H), 7.22 (t, J=7.5 Hz, 1H), 7.29 (d, J=7.8 Hz, 1H), 10.49 (s, 1H); MS (ES+) m/z 262 (parent ion)⁺.

### Intermediate 10i

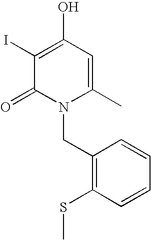

### 1-(2-(methylthio)benzyl)-4-hydroxy-3-iodo-6-methylpyridin-2(1H)-one

Under an argon atmosphere 1-(2-(methylthio)benzyl)-4-hydroxy-6-methylpyridin-2(1H)-one (5.98 g, 21.4 mmol) and N-iodosuccinimide (6.26 g, 27.8 mmol) were stirred in 130 mL anh. acetonitrile overnight at r.t. The mixture was then filtered and the solids washed with anh. acetonitrile (2×10 mL), then by anh. diethyl ether (2×10 mL). The product was then dried under vacuo to give 8.43 g (96% yield) as a tan solid. ¹H NMR (400 MHz, DMSO-d6) δ ppm 2.08 (s, 3H), 2.49 (s, 3H), 5.13 (s, 2H), 5.96 (s, 1H), 6.38 (d, J=7.5 Hz, 1H), 7.06 (t, J=7.4 Hz, 1H), 7.24 (t, J=7.5 Hz, 1H), 7.30 (d, J=7.8 Hz, 1H), 11.40 (s, 1H); MS (ES+) m/z 388 (parent ion)⁺.

### Intermediate 11i

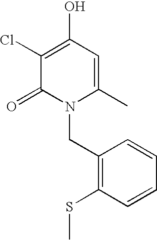

### 1-(2-(methylthio)benzyl)-3-chloro-4-hydroxy-6-methylpyridin-2(1H)-one

Under an argon atmosphere 1-(2-(methylthio)benzyl)-4-hydroxy-3-iodo-6-methylpyridin-2(1H)-one (8.38 g, 20.4 mmol), lithium chloride (6.93 g, 163 mmol), and 100 mL anhydrous DMF were heated at 90° C. for 1.0 hrs. After cooling to r.t., the mixture was concentrated to one third volume by sweeping nitrogen over the surface. The viscous mixture was then added dropwise to 1000 mL water with stirring. The mixture was then filtered, and the product washed with water (3×100 mL), then by acetone (1×15 mL). The product was then dried under vacuo to give 6.22 g (yield 93%) as a tan solid. ¹H NMR (400 MHz, DMSO-d6) δ ppm 2.09 (s, 3H), 2.50 (s, 3H), 5.10 (s, 2H), 6.01 (s, 1H), 6.39 (d, J=7.5 Hz, 1H), 7.06 (t, J=7.4 Hz, 1H), 7.24 (t, J=7.4 Hz, 1H), 7.31 (d, J=7.8 Hz, 1H), 11.26 (s, 1H); MS (ES+) m/z 296 (parent ion)⁺.

### Intermediate 12i

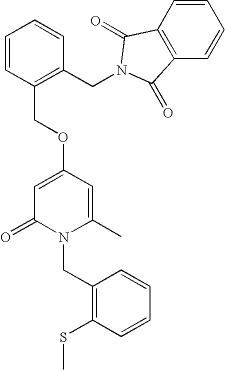

### 2-(2-((1-(2-(methylthio)benzyl)-6-methyl-2-oxo-1,2-dihydropyridin-4-yloxy)methyl)benzyl)isoindoline-1,3-dione

Under a nitrogen atmosphere 1-(2-(methylthio)benzyl)-4-hydroxy-6-methylpyridin-2(1H)-one (3.95 g, 14.5 mmol), 2-[2-(chloromethyl)benzyl]-1H-isoindole-1,3(2H)-dione (5.38 g, 18.8 mmol), anh. potassium carbonate (2.60 g, 18.8 mmol), and 90 mL anh. DMF were heated at 60° C. for 16 hrs. After cooling to r.t., the mixture was added to 1.5 L water with stirring. The resulting precipitate was then filtered, washed with water (3×100 mL) then acetone (2×25 mL), and then dried under vacuo. This gave 7.59 g (97% yield) as a light tan solid. ¹H NMR (400 MHz, DMSO-d6) δ ppm 2.02 (s, 3H), 2.50 (s, 3H), 4.84 (s, 2H), 5.06 (s, 2H), 5.19 (s, 2H), 5.73 (d, J=2.1 Hz, 1H), 5.93 (d, J=2.4 Hz, 1H), 6.44 (d, J=7.5 Hz, 1H), 7.09 (t, J=7.4 Hz, 1H), 7.21-7.35 (m, 5H), 7.41-7.47 (m, 1H), 7.75-7.87 (m, 4H); MS (ES+) m/z 511 (parent ion)⁺.

### Intermediate 13i

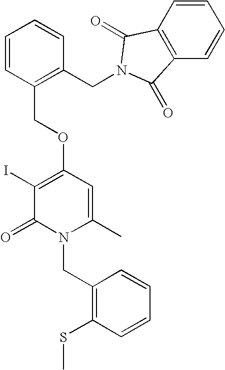

### 2-(2-((1-(2-(methylthio)benzyl)-1,2-dihydro-3-iodo-6-methyl-2-oxopyridin-4-yloxy)methyl)benzyl)isoindoline-1,3-dione

2-(2-((1-(2-(methylthio)benzyl)-1,2-dihydro-3-iodo-6-methyl-2-oxopyridin-4-yloxy)methyl)benzyl)isoindoline-1,3-dione was made in similar manner, except 1-(2-(methylthio)benzyl)-4-hydroxy-3-iodo-6-methylpyridin-2(1H)-one was used instead of 1-(2-(methylthio)benzyl)-4-hydroxy-6-methylpyridin-2(1H)-one. ¹H NMR (400 MHz, DMSO-d6) δ ppm 2.22 (s, 3H), 2.51 (s, 3H), 4.89 (s, 2H), 5.20 (s, 2H), 5.46 (s, 2H), 6.40 (d, J=7.5 Hz, 1H), 6.54 (s, 1H), 7.08 (t, J=7.4 Hz, 1H), 7.20-7.36 (m, 5H), 7.53-7.60 (m, 1H), 7.77-7.88 (m, 4H) m/z 637 (parent ion)⁺.

### Intermediate 14i

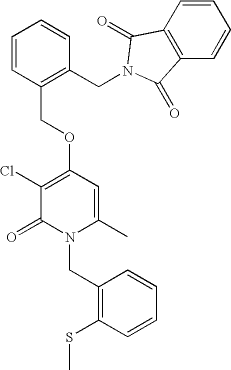

### 2-(2-((1-(2-(methylthio)benzyl)-3-chloro-1,2-dihydro-6-methyl-2-oxopyridin-4-yloxy)methyl)benzyl)isoindoline-1,3-dione

2-(2-((1-(2-(methylthio)benzyl)-3-chloro-1,2-dihydro-6-methyl-2-oxopyridin-4-yloxy)methyl)benzyl)isoindoline-1,3-dione was made in similar manner, except 1-(2-(methylthio)benzyl)-3-chloro-4-hydroxy-6-methylpyridin-2(1H)-one was used instead of 1-(2-(methylthio)benzyl)-4-hydroxy-6-methylpyridin-2(1H)-one. ¹H NMR (400 MHz, DMSO-d6) δ ppm 2.23 (s, 3H), 2.51 (s, 3H), 4.89 (s, 2H), 5.17 (s, 2H), 5.45 (s, 2H), 6.41 (d, J=7.8 Hz, 1H), 6.65 (s, 1H), 7.08 (t, J=7.5 Hz, 1H), 7.21-7.36 (m, 5H), 7.51 (dd, J=5.2, 3.6 Hz, 1H), 7.77-7.85 (m, 4H) m/z 545 (parent ion)⁺.

### Intermediate 15i

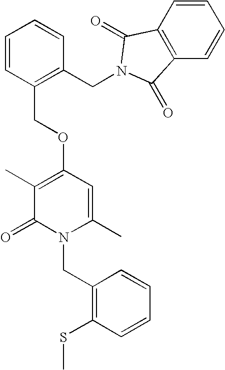

### 2-(2-((1-(2-(methylthio)benzyl)-3,6-dimethyl-2-oxo-1,2-dihydropyridin-4-yloxy)methyl)benzyl)isoindoline-1,3-dione

Under a nitrogen atmosphere 2-(2-((1-(2-(methylthio)benzyl)-1,2-dihydro-3-iodo-6-methyl-2-oxopyridin-4-yloxy)methyl)benzyl)isoindoline-1,3-dione (20.9 g, 31.4 mmol), lithium chloride (4.22 g, 99.6 mmol), 210 mL anhydrous DMF, [1,1-bis(diphenylphosphino)ferrocene]dichloropalladium(II) complex with dichloromethane (1:1) (2.61 g, 3.20 mmol), and tetramethyltin (10.0 mL, 72.5 mmol) were heated at 120° C. for 1 hr. The mixture was allowed to cool to r.t., then added to 2.0 L water with vigorous stirring. Approximately 10 mL of 2.5 N sodium hydroxide was added, and after 5 min. vigorous stirring the slurry was filtered, washed with water (3×100 mL) and then by acetone (3×25 mL). The solid was dried under vacuo overnight at 45-50° C., which gave 14.7 g (yield 81%) as a brown solid. ¹H NMR (400 MHz, DMSO-d6) δ ppm 1.72 (s, 3H), 2.17 (s, 3H), 2.51 (s, 3H), 4.88 (s, 2H), 5.13 (s, 2H), 5.32 (s, 2H), 6.30-6.52 (m, 2H), 7.01-7.12 (m, 1H), 7.17-7.37 (m, 5H), 7.48 (br s, 1H), 7.82 (s, 4H); MS (ES+) m/z 525 (parent ion)⁺.

### Intermediate 16i

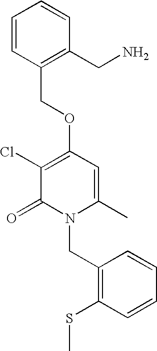

### 4-(2-(aminomethyl)benzyloxy)-1-(2-(methylthio)benzyl)-3-chloro-6-methylpyridin-2(1H)-one

Under a nitrogen atmosphere 2-(2-((1-(2-(methylthio)benzyl)-3-chloro-1,2-dihydro-6-methyl-2-oxopyridin-4-yloxy)methyl)benzyl)isoindoline-1,3-dione (11.67 g, 19.73 mmol), 750 mL methanol, and hydrazine hydrate (37 mL, 761 mmol) were heated at 55-60° C. for 1 hour. The mixture was allowed to cool to r.t., and the methanol removed in vacuo. The residue was partitioned between ethyl acetate and 2.5 N sodium hydroxide. The ethyl acetate was then washed three times with water, dried over magnesium sulfate, and the solvent removed in vacuo. The product was then dried in a vacuo at 50° C. overnight, which gave 7.57 g (yield 84%) as a pink solid. ¹H NMR (400 MHz, DMSO-d6) δ ppm 1.86 (br s, 2H), 2.20 (s, 3H), 2.51 (s, 3H), 3.78 s, 2H), 5.16 (s, 2H), 5.35 (s, 2H), 6.39 (d, J=7.5 Hz, 1H), 6.64 (s, 1H), 7.07 (td, J=7.5 1.1 Hz, 1H), 7.20-7.35 (m, 4H), 7.43 (t, J=7.1 Hz, 2H); MS (ES+) m/z 415 (parent ion)⁺.

### Intermediate 17i

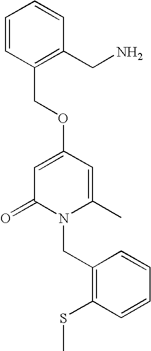

### 4-(2-(aminomethyl)benzyloxy)-1-(2-(methylthio)benzyl)-6-methylpyridin-2(1H)-one

4-(2-(aminomethyl)benzyloxy)-1-(2-(methylthio)benzyl)-6-methylpyridin-2(1H)-one was made in similar manner, except 2-(2-((1-(2-(methylthio)benzyl)-6-methyl-2-oxo-1,2-dihydropyridin-4-yloxy)methyl)benzyl)isoindoline-1,3-dione was used instead of 2-(2-((1-(2-(methylthio)benzyl)-3-chloro-1,2-dihydro-6-methyl-2-oxopyridin-4-yloxy)methyl)benzyl)isoindoline-1,3-dione. ¹H NMR (400 MHz, DMSO-d6) δ ppm 2.08 (s, 3H) 2.35 (br s, 2H) 2.50 (s, 3H) 3.75 (s, 2H) 5.10 (d, J=28.2 Hz, 4H) 5.96 (dd, J=26.9, 2.3 Hz, 2H) 6.43 (d, J=7.8 Hz, 1H) 7.07 (t, J=7.5 Hz, 1H) 7.23 (q, J=7.3 Hz, 2H) 7.27-7.33 (m, 2H) 7.40 (dd, J=27.3, 7.4 Hz, 2H); m/z 381 (parent ion)⁺.

### Intermediate 18i

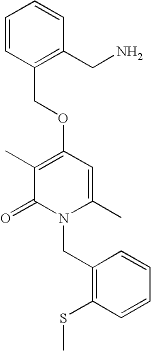

### 4-(2-(aminomethyl)benzyloxy)-1-(2-(methylthio)benzyl)-3,6-dimethylpyridin-2(1H)-one

4-(2-(aminomethyl)benzyloxy)-1-(2-(methylthio)benzyl)-3,6-dimethylpyridin-2(1H)-one was made in similar manner, except 2-(2-((1-(2-(methylthio)benzyl)-3,6-dimethyl-2-oxo-1,2-dihydropyridin-4-yloxy)methyl)benzyl)isoindoline-1,3-dione was used instead of 2-(2-((1-(2-(methylthio)benzyl)-3-chloro-1,2-dihydro-6-methyl-2-oxopyridin-4-yloxy)methyl)benzyl)isoindoline-1,3-dione. ¹H NMR (400 MHz, DMSO-d6) δ ppm 1.69-1.90 (m, 5H), 2.14 (s, 3H), 2.50 (s, 3H), 3.77 (s, 2H), 5.13 (s, 2H), 5.23 (s, 2H), 6.36 (d, J=7.8 Hz, 1H), 6.43 (s, 1H), 7.05 (t, J=7.0 Hz, 1H), 7.19-7.26 (m, 2H), 7.26-7.33 (m, 2H), 7.41 (dd, J=16.1, 7.5 Hz, 2H); m/z 395 (parent ion)⁺.

### Intermediate 19i

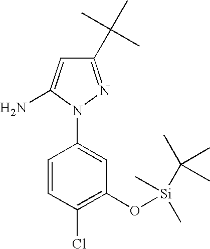

### 3-tert-butyl-1-(3-(tert-butyldimethylsilyloxy)-4-chlorophenyl)-1H-pyrazol-5-amine

Under an argon atmosphere, 5-(5-amino-3-tert-butyl-1H-pyrazol-1-yl)-2-chlorophenol, (3.45 g, 13.0 mmol), 11.0 mL anh. DMF, t-butyldimethylsilyl chloride (2.35 g, 15.6 mmol), and imidazole (2.22 g, 32.6 mmol) were stirred for 19 hrs. at r.t. Then the reaction mixture was added to 250 mL of aqueous 5% sodium bicarbonate solution, filtered, the product washed with water (2×100 mL), and then dried under vacuum to give 4.88 g (yield 96%) a yellow solid. ¹H NMR (400 MHz, DMSO-d6) δ ppm 0.20 (s, 6H), 0.96 (s, 9H), 1.16 (s, 9H), 5.21 (s, 2H), 5.35 (s, 1H), 7.21 (dd, J=8.6, 2.4 Hz, 1H), 7.27 (d, J=2.4 Hz, 1H), 7.42 (d, J=8.9 Hz, 1H); MS (ES+) m/z 380 (parent ion)⁺.

### Intermediate 20i

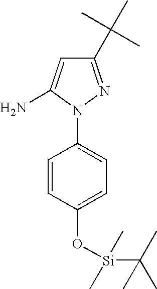

### 3-tert-butyl-1-(4-tert-butyldimethylsilyloxyphenyl)-1H-pyrazol-5-amine

3-tert-butyl-1-(4-tert-butyldimethylsilyloxyphenyl)-1H-pyrazol-5-amine was made in similar fashion, except, 4-(5-amino-3-tert-butyl-1H-pyrazol-1-yl)phenol, was used instead of 5-(5-amino-3-tert-butyl-1H-pyrazol-1-yl)-2-chlorophenol. ¹H NMR (400 MHz, DMSO-d6) δ ppm 0.16 (s, 6H), 0.92 (s, 9H), 1.15 (s, 9H), 5.01 (s, 2H), 5.29 (s, 1H), 6.85 (d, J=8.6 Hz, 2H), 7.35 (d, J=8.6 HZ, 2H); m/z 346 (parent ion)⁺.

### Intermediate 21i

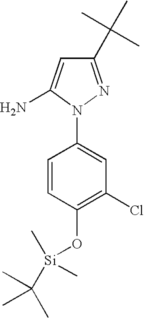

### 3-tert-butyl-1-(4-(tert-butyldimethylsilyloxy)-3-chlorophenyl)-1H-pyrazol-5-amine

3-tert-butyl-1-(4-(tert-butyldimethylsilyloxy)-3-chlorophenyl)-1H-pyrazol-5-amine was made in similar fashion, except 4-(5-amino-3-tert-butyl-1H-pyrazol-1-yl)-2-chlorophenol, was used instead of 5-(5-amino-3-tert-butyl-1H-pyrazol-1-yl)-2-chlorophenol. ¹H NMR (400 MHz, DMSO-d6) δ ppm 0.20 (s, 6H), 0.96 (s, 9H), 1.15 (s, 9H), 5.15 (s, 2H), 5.32 (s, 1H), 7.02 (d, J=8.9 Hz, 1H), 7.38 (dd, J=8.7, 2.6 Hz, 1H), 7.55 (d, J=2.7 Hz, 1H); m/z 380 (parent ion)⁺.

### Intermediate 22i

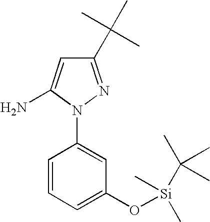

### 3-tert-butyl-1-(3-tert-butyldimethylsilyloxyphenyl)-1H-pyrazol-5-amine

3-tert-butyl-1-(3-tert-butyldimethylsilyloxyphenyl)-1H-pyrazol-5-amine was made in similar fashion, except 3-(5-amino-3-tert-butyl-1H-pyrazol-1-yl)phenol, was used instead of 5-(5-amino-3-tert-butyl-1H-pyrazol-1-yl)-2-chlorophenol, and additional t-butyldimethylsilyl chloride (0.63 g, 4.2 mmol) was added after 19 hrs. After stirring the weekend at r.t., the mixture the added to 250 mL of aqueous 5% sodium bicarbonate solution, and the product extracted with pet. ether. The pet. ether was removed in vacuo and the product dried under vacuum to give 4.62 g (yield 99%) a viscous brown oil. ¹HNMR (400 MHz, DMSO-d6) δ ppm 0.17 (s, 6H), 0.92 (s, 9H), 1.16 (s, 9H), 5.13 (s, 2H), 5.33 (s, 1H), 6.69 (dd J=7.9, 1.5 HZ, 1H), 7.05 (t, J=2.1 Hz, 1H), 7.16 (d, J=9.1 Hz, 1H), 7.25 (t, J=8.1 Hz, 1H); m/z 346 (parent ion)⁺.

### Intermediate 23i

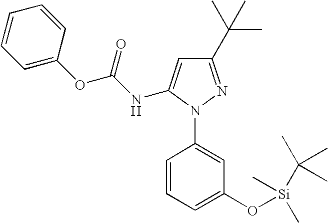

### Phenyl 3-tert-butyl-1-(3-(tert-butyldimethylsilyloxy)phenyl)-1H-pyrazol-5-ylcarbamate

3-tert-butyl-1-(3-tert-butyldimethylsilyloxyphenyl)-1H-pyrazol-5-amine (4.36 g, 12.6 mmol) was dissolved in 210 mL anh. THF, then placed in an ice/water bath and anh. pyridine (1.3 mL, 16 mmol) was added, followed by dropwise addition of phenylchloroformate (2.5 mL, 20 mmol). After 10 min., the reaction was removed from the bath and continued 120 min. at r.t. The reaction was then diluted with 420 mL ethyl acetate and washed with water (2×210 mL). The organic layer was dried over anh. sodium sulfate and the solvents removed in vacuo. This gave 5.4 g (yield 89%) as a light orange solid. MS (ES+) m/z 466 (parent ion)⁺.

### Intermediate 24i

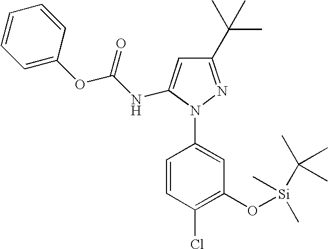

### Phenyl 3-tert-butyl-1-(3-(tert-butyldimethylsilyloxy)-4-chlorophenyl)-1H-pyrazol-5-ylcarbamate

Phenyl 3-tert-butyl-1-(3-(tert-butyldimethylsilyloxy)-4-chlorophenyl)-1H-pyrazol-5-ylcarbamate was made in similar fashion; except 3-tert-butyl-1-(3-(tert-butyldimethylsilyloxy)-4-chlorophenyl)-1H-pyrazol-5-amine was used instead of 3-tert-butyl-1-(3-tert-butyldimethylsilyloxyphenyl)-1H-pyrazol-5-amine and additional phenylchloroformate (0.15 mL, 1.2 mmol) was required. m/z 500 (parent ion)⁺

### Intermediate-25i

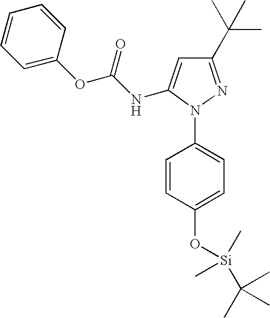

### Phenyl 3-tert-butyl-1-(4-tert-butyldimethylsilyloxyphenyl)-1H-pyrazol-5-ylcarbamate

Phenyl 3-tert-butyl-1-(4-tert-butyldimethylsilyloxyphenyl)-1H-pyrazol-5-ylcarbamate was made in similar fashion, except 3-tert-butyl-1-(4-tert-butyldimethylsilyloxyphenyl)-1H-pyrazol-5-amine was used instead of 3-tert-butyl-1-(3-tert-butyldimethylsilyl-oxyphenyl)-1H-pyrazol-5-amine and additional phenylchloroformate (0.15 mL, 1.2 mmol) was required. m/z 466 (parent ion)⁺.

### Intermediate 26i

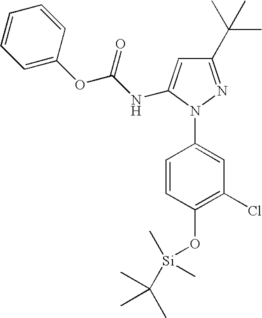

### Phenyl 3-tert-butyl-1-(4-(tert-butyldimethylsilyloxy)-3-chlorophenyl)-1H-pyrazol-5-ylcarbamate

Phenyl 3-tert-butyl-1-(4-(tert-butyldimethylsilyloxy)-3-chlorophenyl)-1H-pyrazol-5-ylcarbamate was made in similar fashion, except 3-tert-butyl-1-(4-(tert-butyldimethylsilyloxy)-3-chlorophenyl)-1H-pyrazol-5-amine was used instead of 3-tert-butyl-1-(3-tert-butyldimethylsilyloxyphenyl)-1H-pyrazol-5-amine. m/z 500 (parent ion)⁺.

### Examples 1-12

### Example 1

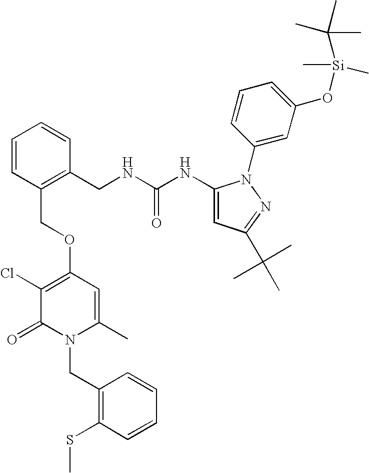

### 1-(2-((1-(2-(methylthio)benzyl)-3-chloro-6-methyl-2-oxo-1,2-dihydropyridin-4-yloxy)methyl)benzyl)-3-(3-tert-butyl-1-(3-(tert-butyldimethylsilyloxy)phenyl)-1H-pyrazol-5-yl)urea

To a suspension of 4-(2-(aminomethyl)benzyloxy)-1-(2-(methylthio)benzyl)-3-chloro-6-methylpyridin-2(1H)-one (0.265 g, 0.554 mmol) in 3.0 mL anh. THF was added triethylamine (0.50 mL, 3.6 mmol), then a suspension of phenyl 3-tert-butyl-1-(3-(tert-butyldimethylsilyloxy)phenyl)-1H-pyrazol-5-ylcarbamate (0.25 g, 0.50 mmol) in 7.0 mL anh. THF, and finally 0.3 g of 3 Å molecular sieves. The reaction was then refluxed for 1.0 hrs. under nitrogen, followed by stirring at r.t. overnight. The reaction was then diluted with enough THF to dissolve everything, but the molecular sieves. The mixture was then filtered and the solvents removed in vacuo to give crude 1-(2-((1-(2-(methylthio)benzyl)-3-chloro-6-methyl-2-oxo-1,2-dihydropyridin-4-yloxy)methyl)-benzyl)-3-(3-tert-butyl-1-(3-(tert-butyldimethylsilyloxy)phenyl)-1H-pyrazol-5-yl)urea. MS (ES+) m/z 786.6 (parent ion)⁺ theoretical exact mass: 785.3198).

The following compounds were synthesized by methods similar to those used in the synthesis of the compound of Example 1:

| Example | | | m/z | ¹H |
|---|---|---|---|---|
| Number | Structure | Name | (parent ion)⁺ | NMR |
| 1 | 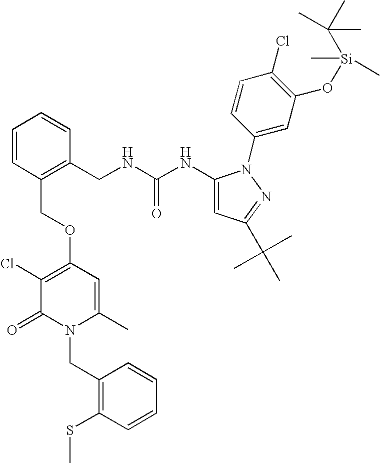 | 1-(2-((1-(2- (methylthio)benzyl)-3-chloro- 6-methyl-2-oxo-1,2- dihydropyridin-4- yloxy)methyl)benzyl)-3-(3- tert-butyl-1-(3-(tert- butyldimethylsilyloxy)-4- chlorophenyl)-1H-pyrazol-5- yl)urea | 820.5 (theoretical exact mass: 819.2808) | Not available |
| 2 | 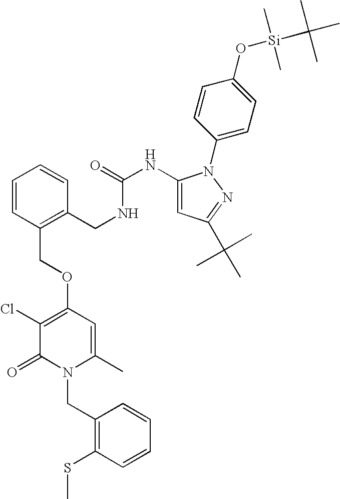 | 1-(2-((1-(2- (methylthio)benzyl)-3-chloro- 6-methyl-2-oxo-1,2- dihydropyridin-4- yloxy)methyl)benzyl)-3-(3- tert-butyl-1-(4-(tert- butyldimethylsilyloxy)phenyl)- 1H-pyrazol-5-yl)urea | 786.6 (theoretical exact mass: 785.3198) | Not available |
| 3 | 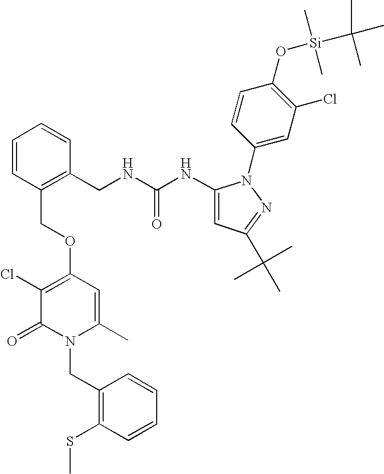 | 1-(2-((1-(2- (methylthio)benzyl)-3-chloro- 6-methyl-2-oxo-1,2- dihydropyridin-4- yloxy)methyl)benzyl)-3-(3- tert-butyl-1-(4-(tert- butyldimethylsilyloxy)-3- chlorophenyl)-1H-pyrazol-5- yl)urea | 820.5 (theoretical exact mass: 819.2808) | Not available |
| 4 | 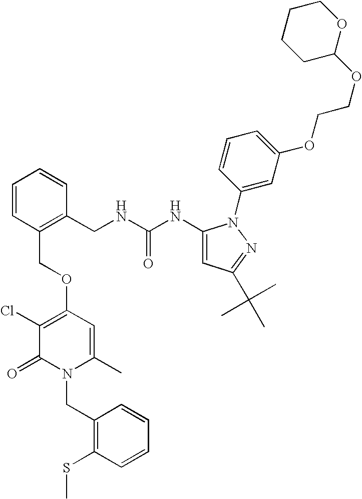 | 1-(2-((1-(2- (methylthio)benzyl)-3-chloro- 6-methyl-2-oxo-1,2- dihydropyridin-4- yloxy)methyl)benzyl)-3-(3- tert-butyl-1-(3-(2-tetrahydro- 2H-pyran-2- yloxy)ethoxy)phenyl)-1H- pyrazol-5-yl)urea | 822.6 (theoretical exact mass: 799.317) | Not available |
| 5 | 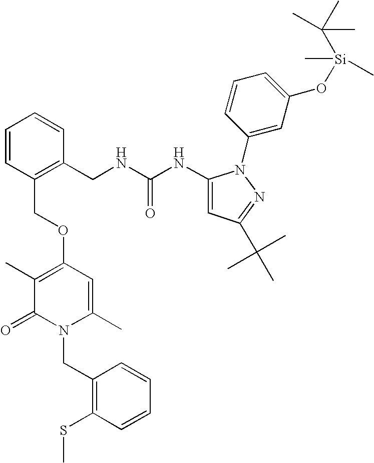 | 1-(2-((1-(2- (methylthio)benzyl)-3,6- dimethyl-2-oxo-1,2- dihydropyridin-4- yloxy)methyl)benzyl)-3-(3- tert-butyl-1-(3-(tert- butyldimethylsilyloxy)phenyl)- 1H-pyrazol-5-yl)urea | 766.6 (theoretical exact mass: 765.3744) | Not available |
| 6 | 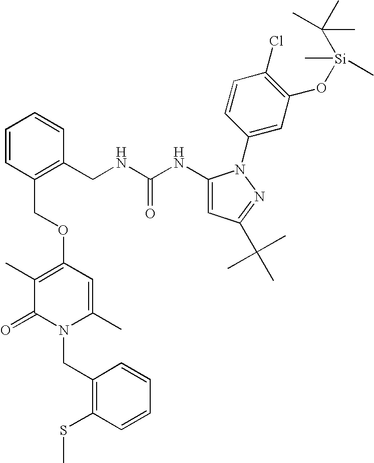 | 1-(2-((1-(2- (methylthio)benzyl)-3,6- dimethyl-2-oxo-1,2- dihydropyridin-4- yloxy)methyl)benzyl)-3-(3- tert-butyl-1-(3-(tert- butyldimethylsilyloxy)-4- chlorophenyl)-1H-pyrazol-5- yl)urea | 800.6 (theoretical exact mass: 799.3354) | Not available |
| 7 | 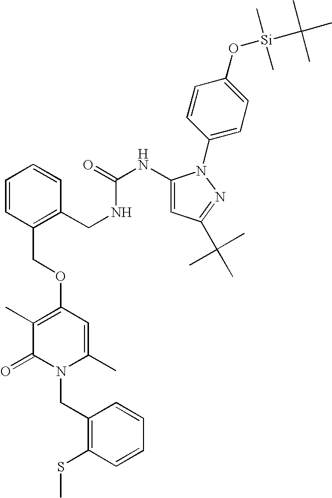 | 1-(2-((1-(2- (methylthio)benzyl)-3,6- dimethyl-2-oxo-1,2- dihydropyridin-4- yloxy)methyl)benzyl)-3-(3- tert-butyl-1-(4-(tert- butyldimethylsilyloxy)phenyl)- 1H-pyrazol-5-yl)urea | 766.6 (theoretical exact mass: 765.3744) | Not available |
| 8 | 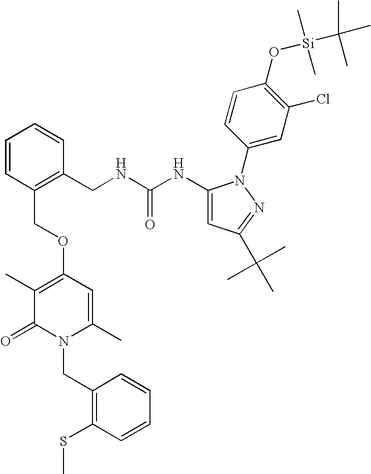 | 1-(2-((1-(2- (methylthio)benzyl)-3,6- dimethyl-2-oxo-1,2- dihydropyridin-4- yloxy)methyl)benzyl)-3-(3- tert-butyl-1-(4-(tert- butyldimethylsilyloxy)-3- chlorophenyl)-1H-pyrazol-5- yl)urea | 800.6 (theoretical exact mass: 799.3354) | Not available |
| 9 | 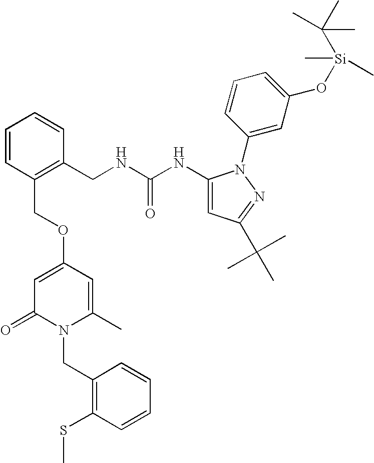 | 1-(2-((1-(2- (methylthio)benzyl)-6-methyl- 2-oxo-1,2-dihydropyridin-4- yloxy)methyl)benzyl)-3-(3- tert-butyl-1-(3-(tert- butyldimethylsilyloxy)phenyl)- 1H-pyrazol-5-yl)urea | 752.6 (theoretical exact mass: 751.3588) | Not available |
| 10 | 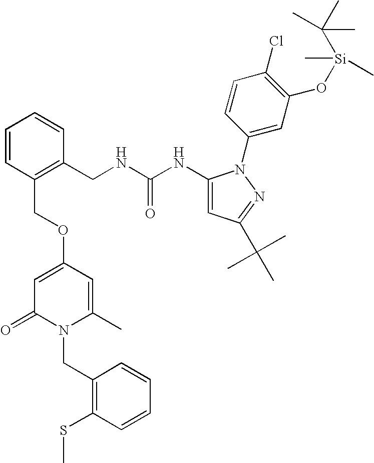 | 1-(2-((1-(2- (methylthio)benzyl)-6-methyl- 2-oxo-1,2-dihydropyridin-4- yloxy)methyl)benzyl)-3-(3- tert-butyl-1-(3-(tert- butyldimethylsilyloxy)-4- chlorophenyl)-1H-pyrazol-5- yl)urea | 786.6 (theoretical exact mass: 785.3198) | Not available |
| 11 | 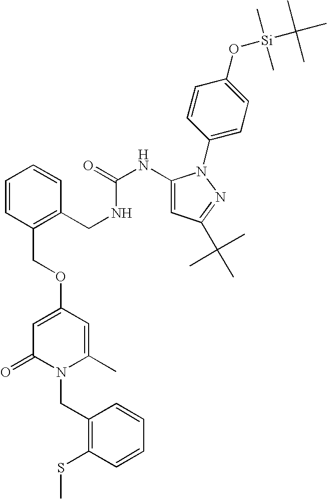 | 1-(2-((1-(2- (methylthio)benzyl)-6-methyl- 2-oxo-1,2-dihydropyridin-4- yloxy)methyl)benzyl)-3-(3- tert-butyl-1-(4-(tert- butyldimethylsilyloxy)phenyl)- 1H-pyrazol-5-yl)urea | 752.6 (theoretical exact mass: 751.3588) | Not available |
| 12 | 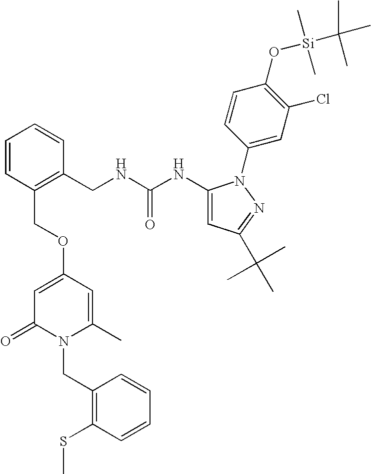 | 1-(2-((1-(2- (methylthio)benzyl)-6-methyl- 2-oxo-1,2-dihydropyridin-4- yloxy)methyl)benzyl)-3-(3- tert-butyl-1-(4-(tert- butyldimethylsilyloxy)-3- chlorophenyl)-1H-pyrazol-5- yl)urea | 786.6 (theoretical exact mass: 785.3198) | Not available |

### Example 13

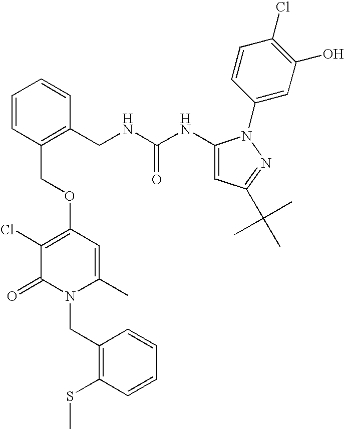

### 1-(2-((1-(2-(methylthio)benzyl)-3-chloro-6-methyl-2-oxo-1,2-dihydropyridin-4-yloxy)methyl)benzyl)-3-(3-tert-butyl-1-(4-chloro-3-hydroxyphenyl)-1H-pyrazol-5-yl)urea

To the crude 1-(2-((1-(2-(methylthio)benzyl)-3-chloro-6-methyl-2-oxo-1,2-di-hydropyridin-4-yloxy)methyl)benzyl)-3-(3-tert-butyl-1-(3-(tert-butyldimethyl-silyloxy)-4-chlorophenyl)-1H-pyrazol-5-yl)urea, was added 10 mL methanol and potassium fluoride (0.091 g, 1.6 mmol, 3 equivalent). After one hour 0.7 mL of 1 N aqueous hydrochloric acid was added, and stirred for 10 min. The solvents were then removed in vacuo and the residue placed under vacuum at 50° C. The residue was then taken up in methylene chloride and methanol and purified by FlashMaster using a 70 g silica column (Isolute) and a hexane/ethyl acetate gradient from 0% ethyl acetate to 50% in 10 min. followed by 50% ethyl acetate to 100% in 30 min. The solvents were then stripped in vacuo to give 0.0393 g (yield 11%) of 1-(2-((1-(2-(methylthio)benzyl)-3-chloro-6-methyl-2-oxo-1,2-dihydropyridin-4-yloxy)methyl)benzyl)-3-(3-tert-butyl-1-(4-chloro-3-hydroxyphenyl)-1H-pyrazol-5-yl)urea. ¹H NMR (400 MHz, DMSO-d₆) δ ppm 1.04 (s, 9H), 2.03 (s, 3H), 2.35 (s, 3H), 4.18 (d, J=5.1 Hz, 2H), 5.03 (s, 2H), 5.18 (s, 2H), 6.05 (s, 1H), 6.26 (d, J=7.3 Hz, 1H), 6.42 (s, 1H), 6.68-6.82 (m, 2H), 6.85-6.99 (m, 2H), 7.05-7.24 (m, 6H), 7.31 (d, J=7.3 Hz, 1H), 8.07 (s, 1H), 10.28 (s, 1H); MS (ES+) m/z 706 (parent ion)⁺.

The following compounds were made using methods similar to those used in Example 13.

### Example 14

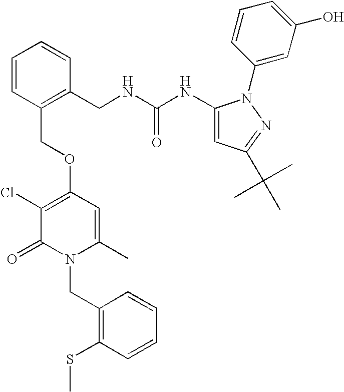

### 1-(2-((1-(2-(methylthio)benzyl)-3-chloro-6-methyl-2-oxo-1,2-dihydropyridin-4-yloxy)methyl)benzyl)-3-(3-tert-butyl-1-(3-hydroxyphenyl)-1H-pyrazol-5-yl)urea

¹H NMR (400 MHz, DMSO-d₆) δ ppm 1.19 (s, 9H), 2.17 (s, 3H), 2.50 (s, 3H), 4.34 (d, J=5.1 Hz, 2H), 5.17 (s, 2H), 5.33 (s, 2H), 6.19 (s, 1H), 6.41 (d, J=7.3 Hz, 1H), 6.57 (s, 1H), 6.72 (d, J=7.3 Hz, 1H), 6.80-6.90 (m, 2H), 6.91-7.00 (m, 1H), 7.06 (t, J=7.3 Hz, 1H), 7.16-7.38 (m, 6H), 7.45 (d, J=7.3 Hz, 1H), 8.17 (s, 1H), 9.65 (s, 1H). MS (ES+) m/z 672 (parent ion)⁺.

### Example 15

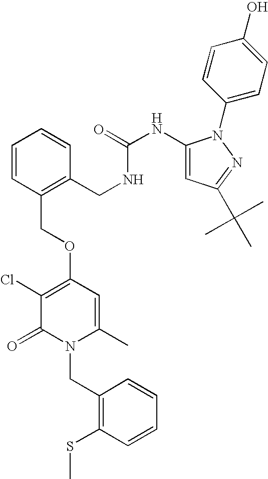

### 1-(2-((1-(2-(methylthio)benzyl)-3-chloro-6-methyl-2-oxo-1,2-dihydropyridin-4-yloxy)methyl)benzyl)-3-(3-tert-butyl-1-(4-hydroxyphenyl)-1H-pyrazol-5-yl)urea

¹H NMR (400 MHz, DMSO-d₆) δ ppm 1.18 (s, 9H), 2.17 (s, 3H), 2.50 (s, 3H), 4.32 (d, J=5.1 Hz, 2H), 5.17 (s, 2H), 5.33 (s, 2H), 6.16 (s, 1H), 6.40 (d, J=7.3 Hz, 1H), 6.57 (s, 1H), 6.79 (d, J=8.8 Hz, 2H), 6.89-6.97 (m, 1H), 7.06 (t, J=7.3 Hz, 1H), 7.16 (d, J=8.8 Hz, 2H), 7.21-7.38 (m, 5H), 7.45 (d, J=6.6 Hz, 1H), 8.01 (s, 1H), 9.62 (s, 1H). MS (ES+) m/z 672 (parent ion)⁺.

### Example 16

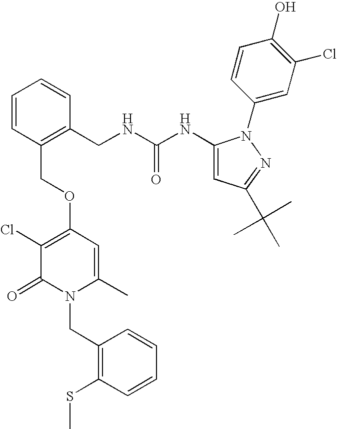

### 1-(2-((1-(2-(methylthio)benzyl)-3-chloro-6-methyl-2-oxo-1,2-dihydropyridin-4-yloxy)methyl)benzyl)-3-(3-tert-butyl-1-(3-chloro-4-hydroxyphenyl)-1H-pyrazol-5-yl)urea

¹H NMR (400 MHz, DMSO-d₆) δ ppm 1.18 (s, 9H), 2.17 (s, 3H), 2.50 (s, 3H), 4.32 (d, J=5.1 Hz, 2H), 5.17 (s, 2H), 5.33 (s, 2H), 6.16 (s, 1H), 6.40 (d, J=7.3 Hz, 1H), 6.57 (s, 1H), 6.86-6.95 (m, 1H), 6.99 (d, J=8.8 Hz, 1H), 7.06 (t, J=7.3 Hz, 1H), 7.13-7.39 (m, 7H), 7.45 (d, J=7.3 Hz, 1H), 8.11 (s, 1H), 10.38 (s, 1H). MS (ES+) m/z 706 (parent ion)⁺. MS (ES+) m/z 672 (parent ion)⁺.

### Example 17

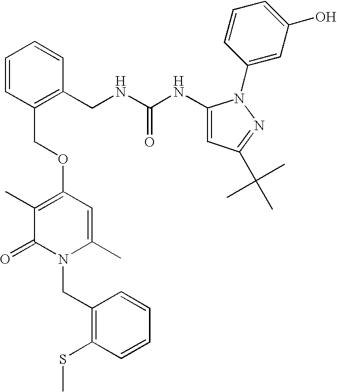

### 1-(2-((1-(2-(methylthio)benzyl)-3,6-dimethyl-2-oxo-1,2-dihydropyridin-4-yloxy)methyl)benzyl)-3-(3-tert-butyl-1-(3-hydroxyphenyl)-1H-pyrazol-5-yl)urea

¹H NMR (400 MHz, DMSO-d₆) δ ppm 1.22 (s, 9H), 1.88 (s, 3H), 2.16 (s, 3H), 2.53 (s, 3H), 4.36 (d, J=5.1 Hz, 2H), 5.18 (s, 2H), 5.25 (s, 2H), 6.22 (s, 1H), 6.34-6.49 (m, 2H), 6.77 (d, J=8.1 Hz, 1H), 6.83-6.92 (m, 2H), 6.94-7.02 (m, 1H), 7.07 (t, J=7.3 Hz, 1H), 7.16-7.40 (m, 6H), 7.46 (d, J=6.6 Hz, 1H), 8.20 (s, 1H), 9.78 (s, 1H). MS (ES+) m/z 652 (parent ion)⁺.

### Example 18

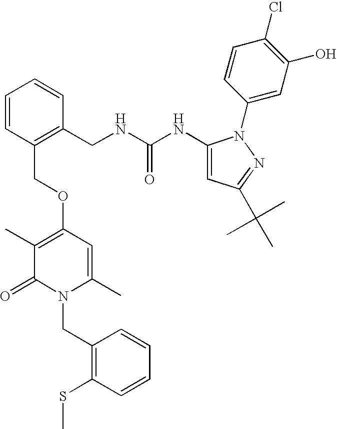

### 1-(2-((1-(2-(methylthio)benzyl)-3,6-dimethyl-2-oxo-1,2-dihydropyridin-4-yloxy)methyl)benzyl)-3-(3-tert-butyl-1-(4-chloro-3-hydroxyphenyl)-1H-pyrazol-5-yl)urea

¹H NMR (400 MHz, DMSO-d₆) δ ppm 1.24 (s, 9H), 1.89 (s, 3H), 2.17 (s, 3H), 2.54 (s, 3H), 4.37 (d, J=5.1 Hz, 2H), 5.19 (s, 2H), 5.26 (s, 2H), 6.25 (s, 1H), 6.35-6.50 (m, 2H), 6.86-7.00 (m, 2H), 7.03-7.17 (m, 2H), 7.21-7.42 (m, 6H), 7.48 (d, J=5.9 Hz, 1H), 8.25 (s, 1H), 10.48 (s, 1H). MS (ES+) m/z 686 (parent ion)⁺.

### Example 19

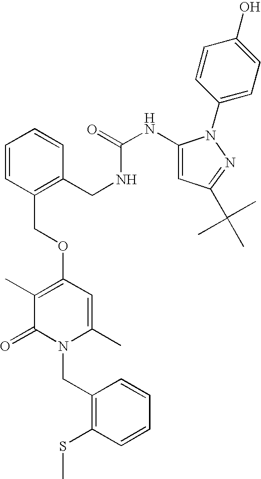

### 1-(2-((1-(2-(methylthio)benzyl)-3,6-dimethyl-2-oxo-1,2-dihydropyridin-4-yloxy)methyl)benzyl)-3-(3-tert-butyl-1-(4-hydroxyphenyl)-1H-pyrazol-5-yl)urea

¹H NMR (400 MHz, DMSO-d₆) δ ppm 1.18 (s, 9H), 1.85 (s, 3H), 2.12 (s, 3H), 2.50 (s, 3H), 4.32 (d, J=5.1 Hz, 2H), 5.14 (s, 2H), 5.21 (s, 2H), 6.17 (s, 1H), 6.32-6.45 (m, 2H), 6.79 (d, J=8.8 Hz, 2H), 6.87-6.95 (m, 1H), 7.04 (t, J=7.0 Hz, 1H), 7.12-7.36 (m, 7H), 7.43 (d, J=6.6 Hz, 1H), 8.01 (s, 1H), 9.62 (s, 1H). MS (ES+) m/z 652 (parent ion)⁺.

### Example 20

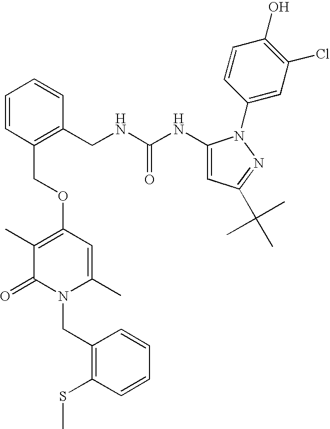

### 1-(2-((1-(2-(methylthio)benzyl)-3,6-dimethyl-2-oxo-1,2-dihydropyridin-4-yloxy)methyl)benzyl)-3-(3-tert-butyl-1-(3-chloro-4-hydroxyphenyl)-1H-pyrazol-5-yl)urea

¹H NMR (400 MHz, DMSO-d₆) δ ppm 1.23 (s, 9H), 1.89 (s, 3H), 2.17 (s, 3H), 2.54 (s, 3H), 4.36 (d, J=5.1 Hz, 2H), 5.18 (s, 2H), 5.25 (s, 2H), 6.21 (s, 1H), 6.37-6.49 (m, 2H), 6.88-6.98 (m, 1H), 7.00-7.13 (m, 2H), 7.17-7.54 (m, 8H), 8.15 (s, 1H), 10.43 (s, 1H). MS (ES+) m/z 686 (parent ion)⁺.

### Example 21

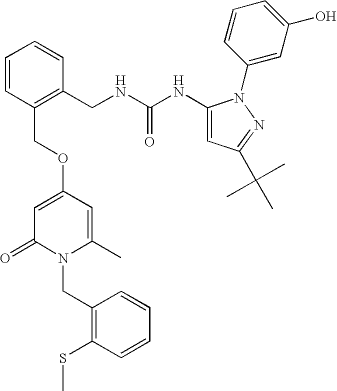

### 1-(2-((1-(2-(methylthio)benzyl)-6-methyl-2-oxo-1,2-dihydropyridin-4-yloxy)methyl)benzyl)-3-(3-tert-butyl-1-(3-hydroxyphenyl)-1H-pyrazol-5-yl)urea

¹H NMR (400 MHz, DMSO-d₆) δ ppm 1.49 (s, 9H), 2.07 (s, 3H), 2.49 (s, 3H), 4.29 (d, J=5.1 Hz, 2H), 5.07 (s, 2H), 5.11 (s, 2H), 5.95 (d, J=19.8 Hz, 2H), 6.19 (s, 1H), 6.44 (d, J=8.1 Hz, 1H), 6.72 (d, J=7.3 Hz, 1H), 6.78-6.96 (m, 3H), 7.05 (t, J=7.3 Hz, 1H), 7.14-7.36 (m, 6H), 7.39 (d, J=6.6 Hz, 1H), 8.16 (s, 1H), 9.71 (s, 1H). MS (ES+) m/z 638 (parent ion)⁺.

### Example 22

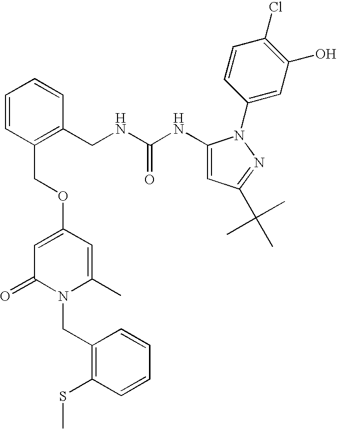

### 1-(2-((1-(2-(methylthio)benzyl)-6-methyl-2-oxo-1,2-dihydropyridin-4-yloxy)methyl)benzyl)-3-(3-tert-butyl-1-(4-chloro-3-hydroxyphenyl)-1H-pyrazol-5-yl)urea

¹H NMR (400 MHz, DMSO-d₆) δ ppm 1.19 (s, 9H), 2.07 (s, 3H), 2.49 (s, 3H), 4.29 (d, J=5.1 Hz, 2H), 5.07 (s, 2H), 5.11 (s, 2H), 5.95 (d, J=22.0 Hz, 2H), 6.20 (s, 1H), 6.44 (d, J=7.3 Hz, 1H), 6.80-6.94 (m, 2H), 6.97-7.14 (m, 2H), 7.15-7.49 (m, 7H), 8.21 (s, 1H), 10.49 (s, 1H). MS (ES+) m/z (parent ion)⁺ not available.

### Example 23

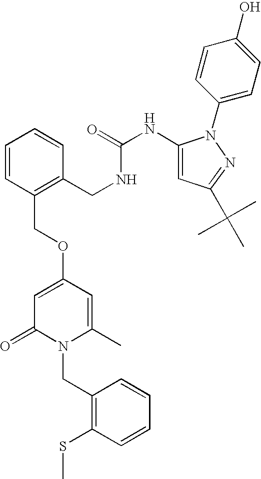

### 1-(2-((1-(2-(methylthio)benzyl)-6-methyl-2-oxo-1,2-dihydropyridin-4-yloxy)methyl)benzyl)-3-(3-tert-butyl-1-(4-hydroxyphenyl)-1H-pyrazol-5-yl)urea

¹H NMR (400 MHz, DMSO-d₆) δ ppm 1.19 (s, 9H), 2.07 (s, 3H), 2.50 (s, 3H), 4.28 (d, J=5.1 Hz, 2H), 5.08 (s, 2H), 5.11 (s, 2H), 5.95 (d, J=17.6 Hz, 2H), 6.17 (s, 1H), 6.45 (d, J=7.3 Hz, 1H), 6.71-6.94 (m, 3H), 7.06 (t, J=7.3 Hz, 1H), 7.11-7.37 (m, 7H), 7.40 (d, J=6.6 Hz, 1H), 8.01 (s, 1H), 9.62 (s, 1H). MS (ES+) m/z 638 (parent ion)⁺.

### Example 24

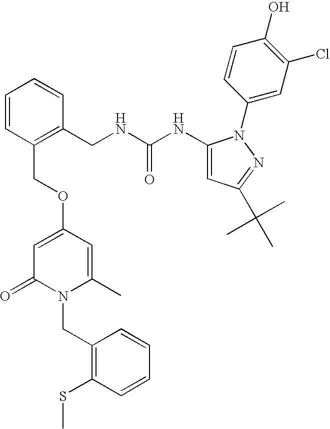

### 1-(2-((1-(2-(methylthio)benzyl)-6-methyl-2-oxo-1,2-dihydropyridin-4-yloxy)methyl)benzyl)-3-(3-tert-butyl-1-(3-chloro-4-hydroxyphenyl)-1H-pyrazol-5-yl)urea

¹H NMR (400 MHz, DMSO-d₆) δ ppm 1.19 (s, 9H), 2.07 (s, 3H), 2.50 (s, 3H), 4.28 (d, J=5.1 Hz, 2H), 5.08 (s, 2H), 5.11 (s, 2H), 5.95 (d, J=16.1 Hz, 2H), 6.17 (s, 1H), 6.45 (d, J=7.3 Hz, 1H), 6.79-6.90 (m, 1H), 7.00 (d, J=8.8 Hz, 1H), 7.06 (t, J=7.3 Hz, 1H), 7.14-7.46 (m, 8H), 8.11 (s, 1H), 10.39 (s, 1H). MS (ES+) m/z 672 (parent ion)⁺.

Compounds of Examples 26-31 were synthesized using methods similar to those used to make compounds of Example 25.

### Example 25

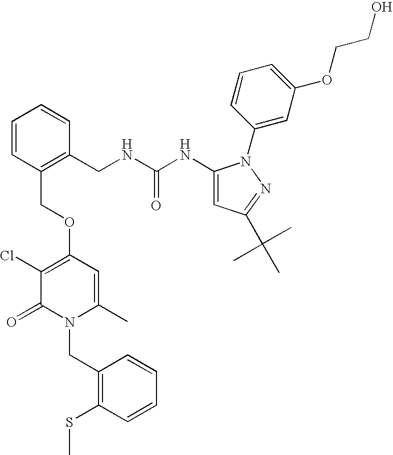

### 1-(2-((1-(2-(methylthio)benzyl)-3-chloro-6-methyl-2-oxo-1,2-dihydropyridin-4-yloxy)methyl)benzyl)-3-(3-tert-butyl-1-(3-(2-hydroxyethoxy)phenyl)-1H-pyrazol-5-yl)urea

To the crude 1-(2-((1-(2-(methylthio)benzyl)-3-chloro-6-methyl-2-oxo-1,2-dihydropyridin-4-yloxy)methyl)benzyl)-3-(3-tert-butyl-1-(3-(2-(tetrahydro-2H-pyran-2-yloxy)ethoxy)phenyl)-1H-pyrazol-5-yl)urea, was added 10 mL methanol and 4-toluenesulfonic acid monohydrate (0.061 g, 0.32 mmol, 0.66 equivalents). The reaction was then stirred at 60° C. for one hour under nitrogen. The solvents were then removed in vacuo and the residue placed under vacuum at 50° C. The crude oil was then purified on silica plates using 5% methanol in methylene chloride. The appropriate level was cut, dissolved, and evaporated. The oil was then triturated with diethyl ether and the resulting solid dried. This gave 0.1228 g (yield 35%) of product.

¹H NMR (400 MHz, DMSO-d₆) δ ppm 1.20 (s, 9H), 2.17 (s, 3H), 2.50 (s, 3H), 3.67 (s, 2H), 3.97 (s, 2H), 4.33 (d, J=5.1 Hz, 2H), 5.17 (s, 2H), 5.33 (s, 2H), 6.21 (s, 1H), 6.40 (d, J=7.3 Hz, 1H), 6.57 (s, 1H), 6.86-7.13 (m, 5H), 7.21-7.39 (m, 6H), 7.45 (d, J=6.6 Hz, 1H), 8.20 (s, 1H); MS (ES+) m/z 716 (parent ion)⁺.

### Intermediate 27i

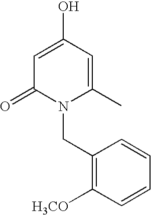

### 1-(2-Methoxybenzyl)-4-hydroxy-6-methylpyridin-2(1H)-one

4-Hydroxy-6-methylpyrone (13.6 g, 107.8 mmol) and 2-methoxybenzylamine were combined with water (300 mL) in a 500 mL round bottom flask, equipped with a reflux condenser, and flushed with nitrogen. The reaction flask was heated in an oil bath at 96° C. for about 13 hours, then at 105° C. for 9 hours. After cooling to just above room temperature, the reaction mixture was filtered, washed with water (75 mL), then with hot water (85° C., 75 mL). The crystals were sequentially dried on the filter, under house vacuum, under a slow flow of nitrogen, and finally under an oil pump vacuum, 23.81 g, 86% yield as a partial hydrate (H₂O)_{0.845}. ¹H NMR (400 MHz, DMSO) δ 2.07 (s, 3H), 3.80 (s, 3H), 5.02 (s, 2H), 6.43 (dd, J=7.52 Hz, 1.34 Hz, 1H), 6.80 (tm, J=7.45 Hz, 1H), 6.97 (d, J=7.65 Hz, 1H), 7.18 (tm, J=7.6 Hz, 1H), 10.43 (s, 1H); MS (ES+) for C₁₄H₁₅NO₃ m/z 246 (M+H)⁺.

### Intermediate 28i

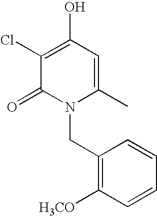

### 1-(2-Methoxybenzyl)-3-chloro-4-hydroxy-6-methylpyridin-2(1H)-one

N-(2-Methoxybenzyl)-4-hydroxy-6-methylpyridinone as a partial (0.845 H₂O) hydrate (5.25 g, 20.15 mmol) was suspended in 1,2-dichloroethane (200 mL) and isopropanol (300 mL), and heated to 55° C., where it dissolved. N-chlorosuccinimide (2.94 g, 22.0 mmol) was added in two portions about one minute apart. After 1.5 h, additional N-chlorosuccinimide (1.0 g, 7.5 mmol) was added. The reaction was removed from the oil bath after 2.5 h total heating time, and the solvent was removed under vacuum and the resulting material triturated overnight with dichloromethane (300 mL). After filtration, the white solid was dried under vacuum with gentle warming, 4.15 g, 11.0 mmol, 54.5% yield, as a pentahydrate (H₂O)_{5.2} by NMR. ¹H NMR (400 MHz, DMSO) δ 2.11 (s, 3H), 3.81 (s, 3H), 5.09 (s, 2H), 5.99 (s, 1H), 6.42 (d, J=6.9 Hz, 1H), 6.81 (t, J=7.5 Hz, 1H), 6.99 (d, J=7.5 Hz, 1H), 7.19 (m, 1H), 11.19 (s, 1H); MS (ES+) for C₁₄H₁₄ClNO₃ m/z 280 (M+H)⁺.

### Intermediate 29i

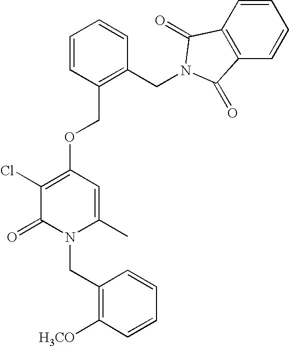

### 2-(2-((1-(2-Methoxybenzyl)-3-chloro-1,2-dihydro-6-methyl-2-oxopyridin-4-yloxy)methyl)benzyl)isoindoline-1,3-dione

1-(2-Methoxybenzyl)-3-chloro-4-hydroxy-6-methylpyridin-2(1H)-one (2.49 g, 6.6 mmol, as a hydrate (H₂O)_{5.2}) was dissolved in anhydrous DMF (50 mL) and 3 Å molecular sieves (0.6 g) were added. After stirring at ambient temperature for a couple of minutes, N-(2-chloromethylbenzyl)phthalimide as a hydrate ((H₂O)_{2.56}, 2.43 g, 7.32 mmol) was added, then potassium carbonate (1.01 g, 7.31 mmol). The flask was flushed with argon, capped with a septum, and stirred overnight at 55° C. After cooling to just above room temperature, the reaction mixture was filtered. The filtrate was reduced in volume to about 15 to 20 mL, then added dropwise to water (900 mL) rapidly stirred. The resulting mixture was stirred for one hour, more water (100 mL) was added, and the mixture was slowly filtered. The precipitate was washed with water (500 mL) and dried under vacuum, 4.2 g, 6.5 mmol, 98%, as a hydrate, (H₂O)_{6.1}, and containing 0.1 equivalents of DMF. ¹H NMR (400 MHz, DMSO) δ 2.30 (s, 3H), 3.87 (s, 3H), 4.93 (s, 2H), 5.20 (s, 2H), 5.48 (s, 2H), 6.49 (d, J=6 Hz, 1H), 6.66 (s, 1H), 6.88 (t, J=7 Hz, 1H), 7.06 (d, J=7.5 Hz, 1H), 7.27 (m, 2H), 7.36 (m, 2H), 7.55 (m, 1H), 7.86 (m. 4H); MS (ES+) for C₃₀H₂₅ClN₂O₅ m/z 529 (M+H)⁺.

### Intermediate 30i

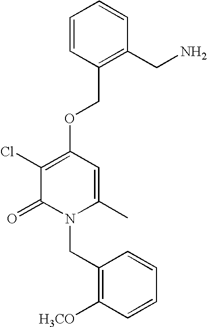

### 4-(2-(Aminomethyl)benzyloxy)-1-(2-methoxybenzyl)-3-chloro-6-methylpyridin-2(1H)-one

2-(2-((1-(2-Methoxybenzyl)-3-chloro-1,2-dihydro-6-methyl-2-oxopyridin-4-yloxy)-methyl)benzyl)isoindoline-1,3-dione (H₂O)_{6.1} (DMF)_{0.1} (2.0 g) was partly dissolved in THF (100 mL) and hydrazine hydrate (1.0 mL) was added. The solution was largely homogeneous. After stirring overnight, the reaction was incomplete by LC/MS. The reaction was heated to 62° C. for 8 h, then more hydrazine hydrate (2.0 mL) was added and the reaction was stirred overnight at about 55° C. Additional hydrazine hydrate (2.0 mL) was added 14 later, and again 8.5 h after that. Additional THF (10 mL) was added with the last aliquot. The reaction was cooled to room temperature 16.5 h after the last addition and the THF was removed under reduced pressure. Ethanol (100 mL) and conc. HCl (2 mL) were added and the heterogeneous mixture was filtered. Additional ethanol (25 mL) was used to rinse the precipitate. As there was some solid in the filtrate, it was refiltered, adding a few mL more ethanol to rinse. Most of the ethanol was removed from the filtrate under reduced pressure and water (75 mL) was added. Ammonium hydroxide (4N) until the pH reached about 10, and the aqueous layer was repeatedly extracted with ethyl acetate. Five extracts of 50 mL and two more extracts of 100 mL contained a common spot by thin layer chromatography and were combined and diluted to 900 mL and dried over sodium sulfate, then magnesium sulfate. After filtration, the solvent was removed on the rotary evaporator, 0.829 g as a hydrate (H₂O)_{0.85}. ¹H NMR (400 MHz, DMSO) δ 2.26 (s, 3H), 3.81 (s, 2H), 3.85 (3H), 5.19 (s, 2H), 5.38 (s, 2H), 6.47 (d, J=7.4 Hz, 1H), 6.64 (s, 1H), 6.86 (t, J=7.7 Hz, 1H), 7.04 (d, J=7.9 Hz, 1H), 7.26 (m, 2H), 7.35 (m, 1H), 7.46 (m, 2H); MS (ES+) for C₂₂H₂₃ClN₂O₃ m/z 399 (M+H)⁺.

Examples 26-29 were made using methods similar to those used in preceeding Example 25.

### Example 26

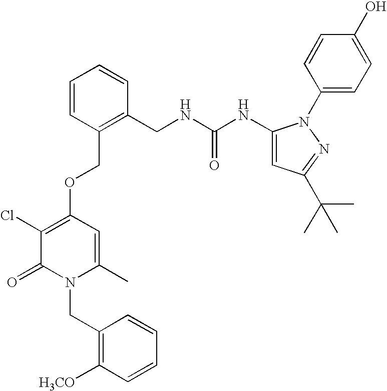

### 1-(2-((1-(2-Methoxybenzyl)-3-chloro-1,2-dihydro-6-methyl-2-oxopyridin-4-yloxy)methyl)benzyl)-3-(3-tert-butyl-1-(4-hydroxyphenyl)-1H-pyrazol-5-yl)urea

Phosgene (20% in toluene, 2.1 mL) was added to dichloromethane (7 mL) in a 100 mL round bottom flask under inert atmosphere stirred in an ice-water bath. 4-(2-(Aminomethyl)benzyloxy)-1-(2-methoxybenzyl)-3-chloro-6-methylpyridin-2(1H)-one as a hydrate (H₂O)_{0.85} (0.271 g, 0.65 mmol) was dissolved in dichloromethane (10 mL) and transferred via cannula to the phosgene solution over one minute. About 3-4 minutes later, a solution of saturated sodium bicarbonate (37 mL) was poured in. After stirring for a total of twenty minutes from the first addition, the reaction mixture was poured into a separatory funnel and the flask rinsed with 10 mL additional dichloromethane. The dichloromethane layer was run onto solid sodium sulfate, swirled for a couple of minutes in an ice-water bath, filtered, and stripped down to a couple of mL of liquid on the rotary evaporator, then placed on a vacuum line, quickly giving a white solid. This solid was dissolved in THF (5 mL) and stirred at 0° C. A solution of 4-(3-tert-butyl-5-amino-1H-pyrazol-1-yl)phenol (0.172 g, 0.74 mmol) in THF (5 mL) was added, and the capped flask was stirred for 1 h at 0° C., then 30 minutes in a cold water bath, and overnight at room temperature. A small amount of precipitate was filtered out of the solution, and the solvent was removed. The residue was chromatographed on silica using ethyl acetate and dichloromethane (25% to 70% ethyl acetate). The appropriate fractions were combined and concentrated under reduced pressure to a white solid in a reddish solution. The solid was filtered, washed with 1:1 ethyl acetate-dichloromethane, and dried under vacuum to give slightly pinkish white solid, 267 mg as a hydrate (H₂O)_{3.2}, 3.7 mmol, 57% yield. ¹H NMR (400 MHz, DMSO) δ 1.21 (s, 9H), 2.23 (s, 3H), 3.85 (s, 3H), 4.35 (d, J=5.6 Hz, 2H), 5.18 (s, 2H), 5.35 (s, 2H), 6.20 (s, 1H), 6.46 (d, J=7.5 Hz, 1H), 6.59 (s, 1H), 6.84 (m, 3H), 6.99 (t, J=5.7 Hz, 1H), 7.04 (d, J=8.2 Hz, 1H), 7.19 to 7.37 (several m, 6H), 7.48 (d, J=7.1 Hz, 1H), 9.71 (s, 1H); MS (ES+) for C₃₆H₃₈ClN₅O₅ m/z 656 (M+H)⁺.

### Example 27

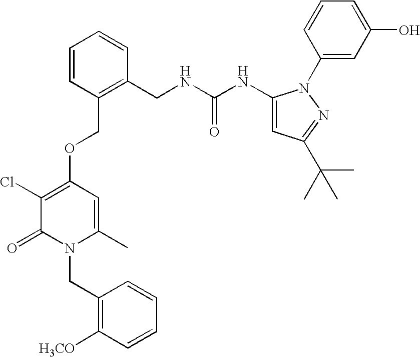

### 1-(2-((1-(2-methoxybenzyl)-3-chloro-1,2-dihydro-6-methyl-2-oxopyridin-4-yloxy)methyl)benzyl)-3-(3-tert-butyl-1-(3-hydroxyphenyl)-1H-pyrazol-5-yl)urea

This compound was prepared in the same manner as for 1-(2-((1-(2-Methoxybenzyl)-3-chloro-1,2-dihydro-6-methyl-2-oxopyridin-4-yloxy)methyl)benzyl)-3-(3-tert-butyl-1-(4-hydroxyphenyl)-1H-pyrazol-5-yl)urea, using 4-(2-(aminomethyl)benzyloxy) 1-(2-methoxybenzyl)-3-chloro-6-methylpyridin-2(1H)-one (H₂O)_{0.85} as one component, and 3-(3-tert-butyl-5-amino-1H-pyrazol-1-yl)phenol as the phenolic pyrazole component. Product was obtained as a nonahydrate and ethyl acetate solvate, 0.028 g, 4% yield. ¹H NMR (400 MHz, DMSO) δ 1.22 (s, 9H), 2.24 (s, 3H), 3.86 (s, 3H), 4.36 (d, J=5.8 Hz, 2H), 6.23 (s, 1H), 6.46 (d, J=6.2 Hz, 1H), 6.76 (m, 1H), 6.86 (m, 3H), 7.03 (m, 2H), 7.22 to 7.36 (m, 5H), 7.48 (d, J=7.3 Hz, 1H), 8.25 (s, 1H), 9.73 (s, 1H); MS (ES+) for C₃₆H₃₈ClN₅O₅ m/z 656 (M+H)⁺.

### Example 28

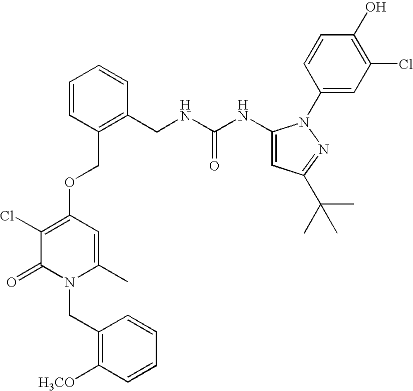

### 1-(2-((1-(2-Methoxybenzyl)-3-chloro-1,2-dihydro-6-methyl-2-oxopyridin-4-yloxy)methyl)benzyl)-3-(3-tert-butyl-1-(3-chloro-4-hydroxyphenyl)-1H-pyrazol-5-yl)urea

4-(2-(Aminomethyl)benzyloxy)-1-(2-methoxybenzyl)-3-chloro-6-methylpyridin-2(1H) (H₂O)_{0.85} (0.200 g, 0.48 mmol) was suspended in THF (5 mL) and triethylamine (0.45 mL, 3.2 mmol) was added followed by phenyl 3-tert-butyl-1-(3-chloro-4-t-butyldimethylsiloxyphenyl)-1H-pyrazol-5-ylcarbamate (H₂O)₄ (0.268 g, 0.455 mmol). The flask was fitted with a reflux condenser and heated under nitrogen at 67° C. for 100 minutes. After cooling to room temperature, tetrabutylammonium fluoride (1M in THF, 0.6 mL, 0.6 mmol) was added and the reaction was stirred for 5 h. At this time, the solvent was removed under reduced pressure. Ethyl acetate (25 mL) was added, then water (15 mL). After shaking the layers were separated and the organic layer was washed once with water (5 mL), with saturated sodium chloride (15 mL), then dried (MgSO₄) and the solvent was evaporated. The residue was chromatographed on silica eluting with an ethyl acetate-dichloromethane gradient giving 41 mg product. ¹H NMR (400 MHz, CD₃OD) δ 1.25 (s, 9H), 2.25 (s, 3H), 3.85 (s, 3H), 4.41 (s, 2H), 5.29 (s, 4H), 6.22 (s, 1H), 6.48 (s, 1H), 6.54 (m, 1H), 6.79 (td, J=7.5 Hz, 0.8 Hz, 1H), 6.94 (m, 2H), 7.13 (dd, J=8.7 Hz, 2.5 Hz, 1H), 7.18 to 7.31 (m, 4H), 7.36 (d, J=2.6 Hz, 1H), 7.45 (m, 1H), 7.83 (s, 1H); MS (ES+) for C₃₆H₃₈ClN₅O₅ m/z 690 (M+H)⁺.

### Example 29

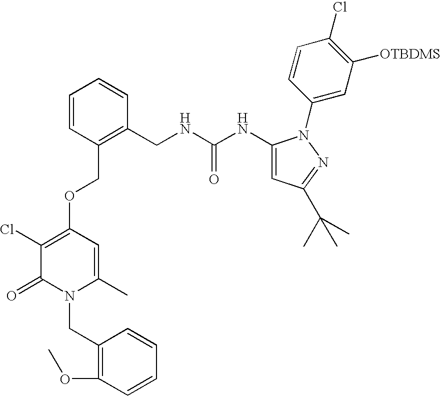

### 1-[3-tert-butyl-1-(3-{[tert-butyl(dimethyl)silyl]oxy}-4-chlorophenyl)-1H-pyrazol-5-yl]-3-[2-({[3-chloro-1-(2-methoxybenzyl)-6-methyl-2-oxo-1,2-dihydropyridin-4-yl]oxy}methyl)benzyl]urea

Triethylamine (0.2 mL, 1.43 mmol) and the appropriate carbamate (0.254 mmol) in THF (2 mL) were added to 4-{[2-(aminomethyl)benzyl]oxy}-3-chloro-1-(3-chloro-4-methoxybenzyl)-6-methylpyridin-2(1H)-one (0.1 g, 0.25 mmol) in THF (2 mL). The reaction mixture was stirred at 60° C. for 2 hours. The solvent was removed to give the appropriate urea, which was carried on without further purification. Yield 0.20 g (97% yield).

The compound of Example 28 was used to make the compound of Example 29. The compounds of Examples 28 and 29 were synthesized using intermediate compounds of the present invention, particularly those of intermediate compounds 31i-34i. Methods for the synthesis of intermediate compounds 31i-34i follow the methods for making Example 29.

### Example 30

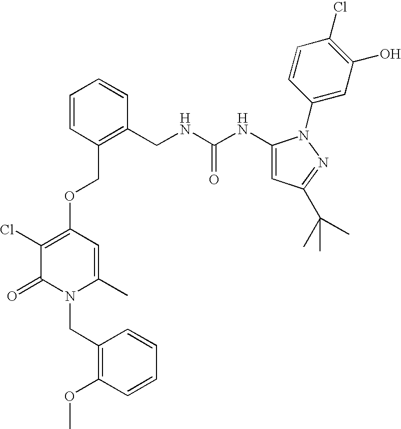

### 1-(2-((1-(2-methoxybenzyl)-3-chloro-1,2-dihydro-6-methyl-2-oxopyridin-4-yloxy)methyl)benzyl)-3-(3-tert-butyl-1-(4-chloro-3-hydroxyphenyl)-1H-pyrazol-5-yl)urea

Potassium fluoride (0.18 g, 3.11 mmol) was added to crude 4-(2-(Aminomethyl)benzyl-oxy)-1-(2-methoxybenzyl)-3,6-dimethylpyridin-2(1H)-one in MeOH (15 mL) and stirred at room temperature for 1.5 h. The solvent was evaporated, the residue was washed with 0.5N HCl followed by water. A preparative chromatography unit (Gilson) with reverse phase was used for purification to give 0.08 g of product as a white solid. ¹H NMR (300 MHz, DMSO-d₆) δ 1.22 (s, 9H), 2.24 (s, 3H), 3.85 (s, 3H), 4.38 (d, 2H, J=5.6 Hz), 5.18 (s, 2H), 5.36 (s, 2H), 6.24 (s, 1H), 6.46 (d, 1H, J=7.4 Hz), 6.59 (s, 1H), 6.80-7.18 (m, 5H), 7.20-7.51 (m, 6H), 8.31 (s, 1H), 10.58 (s, 1H); MS (ES+) for C₃₆H₃₇Cl₂N₅O₅ m/z 690 (M+H)⁺.

### Intermediate 31i

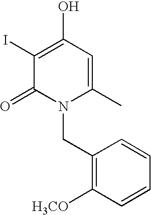

### 1-(2-Methoxybenzyl)-4-hydroxy-3-iodo-6-methylpyridin-2(1H)-one

1-(2-Methoxybenzyl)-4-hydroxy-6-methylpyridin-2(1H)-one (as (H₂O)_{0.845}, 4.98 g, 19.1 mmol) and N-iodosuccinimide were mixed in a round bottom flask and acetonitrile (170 mL) was added, and the mixture was vigorously stirred under nitrogen. After 15.5 h, the reaction mixture was filtered, washed with acetonitrile (30 mL) and ether (20 mL) and dried on the vacuum line giving 7.06 g, as a light grey solid. ¹H NMR (400 MHz, DMSO-d₆) δ 2.10 (s, 3H), 3.81 (s, 3H), 5.12 (s, 2H), 5.93 (s, 1H), 6.41 (dd, J=7.5, 1.3 Hz, 1H), 6.75-6.86 (m, 1H), 6.99 (d, J=7.5 Hz, 1H), 7.14-7.24 (m, 1H); MS (ES+) for C₁₄H₁₄INO₃ m/z 372 [M+H]⁺.

### Intermediate 32i

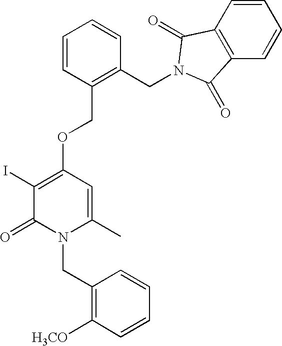

### 2-(2-((1-(2-methoxybenzyl)-1,2-dihydro-3-iodo-6-methyl-2-oxopyridin-4-yloxy)methyl)benzyl)isoindoline-1,3-dione

1-(2-Methoxybenzyl)-4-hydroxy-3-iodo-6-methylpyridin-2(1H)-one (3.0 g, 8.1 mmol), 2-chloromethylbenzylphthalimide (H₂O)_{0.9} (2.63 g, 8.7 mmol), potassium carbonate (1.25 g, 9.0 mmol) and molecular sieves (3 Å, 0.62 g) were mixed in a round bottom flask with DMF (75 mL) and were placed under nitrogen. The reaction was stirred at 57° C. for 14 h. After cooling, it was filtered and the filtrate was concentrated to about 50 mL. The slightly heterogeneous solution was added dropwise or in a small stream to 750 mL of vigorously stirred water, and the milky white suspension was filtered after adding 250 mL additional water. The precipitate was washed with 250 mL water, and the solid dried under vacuum, 4.8 g, as a hydrate (H₂O)_{2.5}. ¹H NMR (400 MHz, DMSO-d₆) δ 2.27 (s, 3H), 3.85 (s, 3H), 4.92 (s, 2H), 5.22 (s, 2H), 5.48 (s, 2H), 6.47 (d, J=6.2 Hz, 1H), 6.86 (m, 1H), 7.04 (d, J=7.7H, 1H), 7.22 to 7.35 (m, 4H), 7.59 (m, 1H), 7.82 to 7.89 (m, 4H); MS (ES+) for C₃₀H₂₅IN₂O₅ m/z 621 [M+H]⁺.

### Intermediate 33i

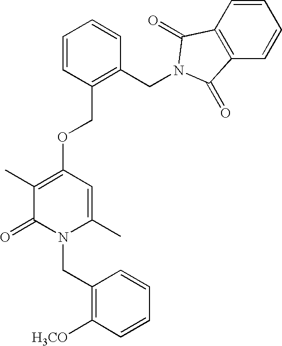

### 2-(2-((1-(2-methoxybenzyl)-1,2-dihydro-3,6-dimethyl-2-oxopyridin-4-yloxy)methyl)benzyl)isoindoline-1,3-dione

Lithium chloride (0.78 g, 18.4 mmol) was placed in a 250 mL round bottom flask and heated to 100-108° C. under oil pump vacuum for a few hours, then nitrogen was admitted after cooling to room temperature. 2-(2-((1-(2-Methoxybenzyl)-1,2-dihydro-3-iodo-6-methyl-2-oxopyridin-4-yloxy)methyl)benzyl)isoindoline-1,3-dione (H₂O)_{2.5} (3.63 g, 5.45 mmol), [1,1′-bis(diphenylphosphino)ferrocene]-dichloropalladium (CH₂Cl₂) (0.467 g, 0.572 mmol), and molecular sieves (3 Å, 0.63 g) were added along with a magnetic stir bar, a vacuum was drawn, and nitrogen was readmitted. DMF (anhydrous, 40 mL) was added via syringe, then tetramethyltin (1.8 mL, 1.31 mmol). The reaction was heated at 123° C. for 25 minutes, then cooled to room temperature. The reddish reaction mixture was filtered through celite, and the filtrate was concentrated to about 30 mL and added dropwise to vigorously stirred water (350 mL). After stirring for about 20 minutes this mixture was filtered and washed with water (350 mL). The dried filter cake was triturated with 25 mL of 10% isopropanol in water, filtered, and the precipitate triturated with 20% isopropanol in water. After filtration, repetition of the last trituration, then drying under vacuum, 2.79 g were obtained. The purity was estimated at about 85% from NMR, and was used without further purification. ¹H NMR (400 MHz, DMSO-d₆) δ 1.75 (s, 3H), 2.23 (s, 3H), 3.86 (s, 3H), 4.91 (s, 2H), 5.16 (s, 2H), 5.35 (s, 2H), 6.40 (m, 2H), 6.85 (t, J=7.5 Hz, 1H), 7.03 (d, J=8.2 Hz, 1H), 7.22 (m, 1H), 7.32 (m, 1H), 7.50 (m, 1H), 7.85 (m, 4H); MS (ES+) for C₃₁H₂₈N₂O₅ m/z 509 [M+H]⁺.

### Intermediate 34i

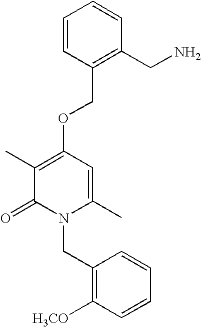

### 4-(2-(Aminomethyl)benzyloxy)-1-(2-methoxybenzyl)-3,6-dimethylpyridin-2(1H)-one

2-(2-((1-(2-methoxybenzyl)-1,2-dihydro-3,6-dimethyl-2-oxopyridin-4-yloxy)methyl)-benzyl)isoindoline-1,3-dione (H₂O)₄ (2.2 g, 3.8 mmol) was suspended in methanol (50 mL) and stirred while hydrazine hydrate (2.5 mL, 51.4 mmol) was added. The solution was homogeneous within 10 minutes; substantial product formation was evident after 25 minutes by LC/MS (MS (ES+) for C₂₃H₂₆N₂O₃ m/z 379 [M+H]⁺). After stirring overnight, the reaction was worked up as in 4-(2-(Aminomethyl)benzyloxy)-1-(2-methoxybenzyl)-3-chloro-6-methylpyridin-2(1H)-one. The product was used as is without further purification.

General Procedures A through M

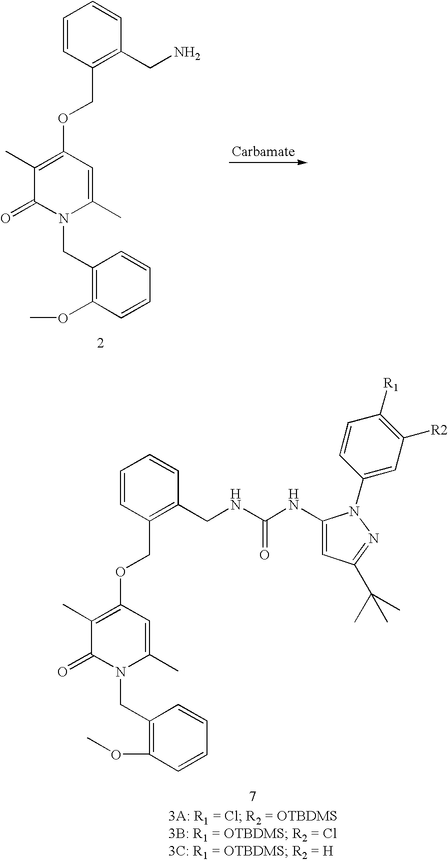

### General Procedure A

Triethylamine (0.2 mL, 1.43 mmol) and the appropriate carbamate (0.254 mmol) in THF (2 mL) were added to 4-(2-(Aminomethyl)benzyloxy)-1-(2-methoxybenzyl)-3,6-dimethylpyridin-2(1H)-one (0.1 g, 0.26 mmol) in THF (2 mL). The reaction mixture was stirred at 60° C. for 2 hours. The liquid part was removed to give the appropriate urea, which was carried on without further purification.

### Example 31

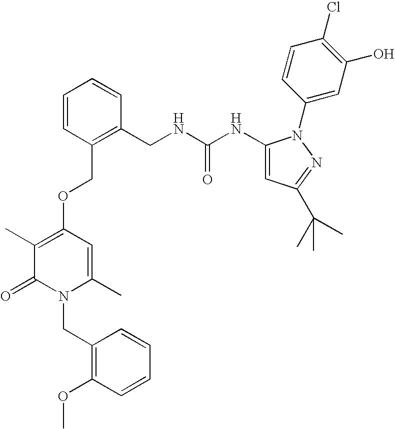

### 1-(2-((1-(2-methoxybenzyl)-1,2-dihydro-3,6-dimethyl-2-oxopyridin-4-yloxy)-methyl)benzyl)-3-(3-tert-butyl-1-(4-chloro-3-hydroxyphenyl)-1H-pyrazol-5-yl)urea

Potassium fluoride (0.18 g, 3.11 mmol) was added to crude 3A in MeOH (15 mL) and stirred at room temperature for 1.5 h. The solvent was evaporated and the residue was washed with 0.5N HCl followed by water. A preparative chromatography unit (Gilson) with reverse phase was used for purification to give 0.077 g of product as a white solid. ¹H NMR (300 MHz, DMSO-d₆) δ 1.23 (s, 9H), 1.88 (s, 3H), 2.28 (s, 3H), 3.85 (s, 3H), 4.38 (d, 2H, J=5.6 Hz), 5.15 (s, 2H), 5.26 (s, 2H), 6.24 (s, 1H), 6.40 (s, 1H), 6.44 (s, 1H), 6.78-7.13 (m, 5H), 7.20-7.50 (m, 6H), 8.21 (s, 1H), 10.58 (s, 1H); MS (ES+) for C₃₇H₄₀ClN₅O₅ m/z 670 (M+H)⁺.

### Example 32

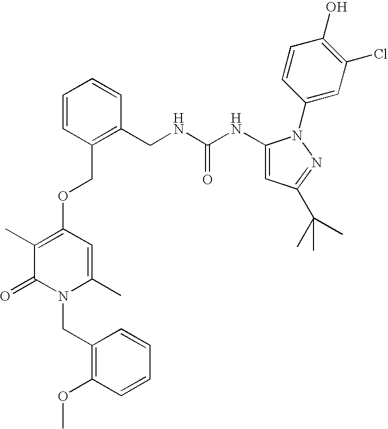

### 1-(2-((1-(2-methoxybenzyl)-1,2-dihydro-3,6-dimethyl-2-oxopyridin-4-yloxy)-methyl)benzyl)-3-(3-tert-butyl-1-(3-chloro-4-hydroxyphenyl)-1H-pyrazol-5-yl)urea

Potassium fluoride (0.18 g, 3.11 mmol) was added to crude 3B in MeOH (15 mL) and stirred at room temperature for 1.5 h. The solvent was evaporated, the residue was washed with 0.5N HCl followed by water. A preparative chromatography unit (Gilson) with reverse phase was used for purification to give 0.075 g of product as a white solid. ¹H NMR (300 MHz, DMSO-d₆) δ 1.21 (s, 9H), 1.87 (s, 3H), 2.21 (s, 3H), 3.85 (s, 3H), 4.35 (d, 2H, J=5.6 Hz), 5.15 (s, 2H), 5.25 (s, 2H), 6.22 (s, 1H), 6.40 (s, 1H), 6.44 (s, 1H), 6.70-7.05 (m, 4H), 7.21-7.48 (m, 7H), 8.20 (s, 1H), 10.50 (s, 1H); MS (ES+) for C₃₇H₄₀ClN₅O₅ m/z 670 (M+H)⁺.

### Example 33

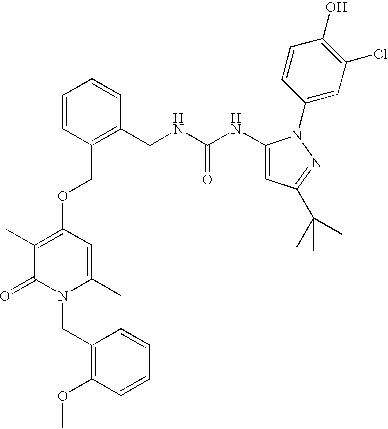

### 1-(2-((1-(2-methoxybenzyl)-1,2-dihydro-3.6-dimethyl-2-oxopyridin-4-yloxy)methyl)benzyl)-3-(3-tert-butyl-1-(4-hydroxyphenyl)-1H-pyrazol-5-yl)urea

Potassium fluoride (0.18 g, 3.11 mmol) was added to crude 3C in MeOH (15 mL) and stirred at room temperature for 1.5 h. The solvent was evaporated, the residue was washed with 0.5N HCl followed by water. A preparative chromatography unit (Gilson) with reverse phase was used for purification to give 0.060 g of product as a white solid. ¹H NMR (300 MHz, DMSO-d₆) δ 1.21 (s, 9H), 1.84 (s, 3H), 2.21 (s, 3H), 3.85 (s, 3H), 4.35 (d, 2H, J=5.6 Hz), 5.15 (s, 2H), 5.24 (s, 2H), 6.20 (s, 1H), 6.40 (s, 1H), 6.43 (s, 1H), 6.56-6.78 (m, 3H), 6.80-7.05 (m, 2H), 6.15-7.40 (m, 7H), 7.42-7.50 (s, 1H), 8.10 (s, 1H), 10.68 (s, 1H); MS (ES+) for C₃₇H₄₁N₅O₅ m/z 636 (M+H)⁺.

### Intermediate 35i

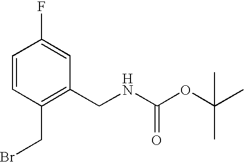

### tert-butyl 2-(bromomethyl)-5-fluorobenzylcarbamate

### Step 1 Synthesis of 2-(bromomethyl)-5-fluorobenzaldehyde

2-(Bromomethyl)-5-fluorobenzonitrile (10.0 g, 47.0 mmol) was dissolved in CH₂Cl₂ (200 mL). The solution was purged under argon for thirty minutes then cooled to 0° C. in an ice-water bath. A solution of diisobutyl aluminum hydride (50.0 mL, 50.0 mmol of a 10.0 M solution in heptane) was added slowly via syringe over a thirty minute period. Once the addition was complete, the ice-water bath was removed and the reaction stirred at room temperature for three hours. Analysis by GCMS showed no remaining starting material. The reaction mixture was cooled in an ice-water bath. It was then poured into a IL Erlenmeyer flask containing ice (150 g,) and 6 N HBr (100 mL). The mixture was stirred for one hour. Extracted with CH₂Cl₂ (3×200 mL). The combined organic phases were washed with NaHCO₃ (aq.) and brine, then dried over MgSO₄, filtered and evaporated. The brown oil was filtered through a plug of silica gel, and washed with CH₂Cl₂ (200 mL). It was evaporated to afford a brown oil, which solidified upon standing. 9.4 g (92%).

### Step 2. Synthesis of tert-butyl 2-(bromomethyl)-5-fluorobenzylcarbamate

2-(Bromomethyl)-5-fluorobenzaldehyde (4.27 g, 19.7 mmol) was dissolved in toluene (75 mL). tert-Butyl carbamate (4.61 g, 39.4 mmol) triethylsilane (9.16 g, 12.59 mL, 78.8 mmol), and trifluoroacetic acid (8.98 g, 6.07 mL, 78.8 mmol) were added. The reaction was stirred overnight at room temperature. An additional 2 mL of triethylsilane, and 0.5 mL of trifluoroacetic acid were added, and the reaction stirred for an additional 6 hours. The reaction mixture was diluted with ethyl acetate (150 mL) and transferred to a separatory funnel. Extracted with H₂O (100 mL), and brine (100 mL). The organic phase was dried over MgSO₄, filtered, and evaporated. The compound was purified by silica gel chromatography. The resulting solid was further purified by recrystallization from hot ether with decolorizing carbon. (2.5 g, 40%) 1HNMR (400 MHz, DMSO-d6) δ ppm 1.37 (s, 9H) 4.25 (d, J=5.91 Hz, 2H) 4.75 (s, 2H) 6.96 (dd, J=10.20, 2.42 Hz 1H) 7.05 (td, J=8.53, 2.82 Hz, 1H) 7.44 (dd, J=8.32, 5.91 Hz, 2H)

### Example 34

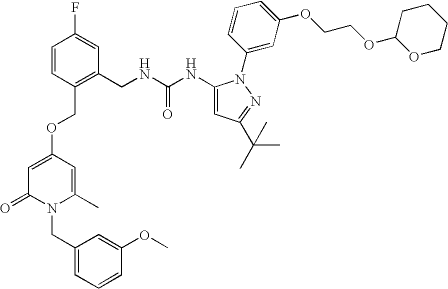

### 1-(3-tert-butyl-1-{3-[2-(tetrahydro-2H-pyran-2-yloxy)ethoxy]phenyl}-1H-pyrazol-5-yl)-3-[5-fluoro-2-({[1-(3-methoxybenzyl)-6-methyl-2-oxo-1,2-dihydropyridin-4-yl]oxy}methyl)benzyl]urea

### Step 1: Synthesis of 1-(3-methoxybenzyl)-4-hydroxy-6-methylpyridin-2(1H)-one

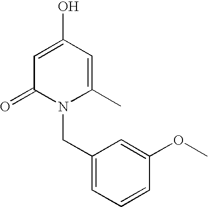

4-hydroxy-6-methyl-2H-pyran-2-one (18.39 g, 146 mmol) and 3-methoxybenzylamine 20 g, 146 mmol) were slurried in H₂O (400 mL). The reaction mixture was stirred at 100° C. for four hours. While hot, the resulting solid was filtered and washed with warm H₂O. The solid was dissolved in 1.25 N NaOH (500 mL) and extracted with CH₂Cl₂ (2×400 mL). The aqueous phase was then neutralized with 6 N HCl, resulting in a yellow precipitate. The solid was filtered, washed with H₂O, and dried under vacuum. (25 g 70%). 1HNMR (400 MHz, DMSO-d6) δ ppm 2.15 (s, 3H) 3.68 (s, 3H) 5.14 (s, 2H) 5.62 (d, J=2.69 Hz, 1H) 5.83 (d, J=2.69 Hz, 1H) 6.50-6.67 (m, 2H) 6.79 (dd, J=8.06, 2.42 Hz, 1H) 7.15-7.27 (m, 1H) HRMS (m/z) 246.1139. M+H, C₁₄H₁₅NO₃ requires 246.1125.

### Step 2: Synthesis of 1-(3-tert-butyl-1-{3-[2-(tetrahydro-2H-pyran-2-yloxy)ethoxy]phenyl}-1H-pyrazol-5-yl)-3-[5-fluoro-2-({[1-(3-methoxybenzyl)-6-methyl-2-oxo-1,2-dihydropyridin-4-yl]oxy}methyl)benzyl]urea

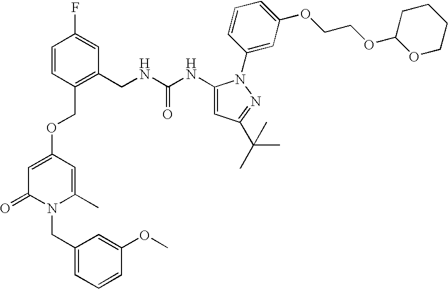

1-(3-methoxybenzyl)-4-hydroxy-6-methylpyridin-2(1H)-one (1.54 g, 6.28 mmol) and tert-butyl 2-(bromomethyl)-5-fluorobenzylcarbamate (2.00 g, 6.28 mmol) were dissolved in DMF (50 mL). DBU (0.939 mL, 0.956 g, 6.28 mmol) was added, and the reaction stirred overnight at room temperature. The reaction mixture was diluted with ethyl acetate, and extracted with H₂O (2×50 mL), and brine (50 mL). The organic phase was dried over MgSO₄, and filtered. The compound was purified by flash column chromatography. A white solid was obtained that was approximately 88% of the desired O-benzylated product and 12% of the undesired C-benzylated product. (1.5 g, 50%). This mixture was carried forward. The mixture (1.5 g, 3.11 mmol) was dissolved in dioxane (25 mL). HCl in dioxane (5 mL of a 4.0 N solution) was added and the reaction stirred over-night at room temperature. The solvent was evaporated, and 0.25 g of the resulting solid was slurried in THF (15 mL). Triethylamine (0.5 mL, 0.36 g, 3.6 mmol) was added, followed by phenyl 3-tert-butyl-1-(3-(2-(tetrahydro-2H-pyran-2-yloxy)ethoxy)-phenyl)-1H-pyrazol-5-ylcarbamate (0.237 g, 0.495 mmol) The reaction was stirred over-night at room temperature. The mixture was diluted with ethyl acetate (50 mL), and extracted with 2.5 N NaOH (2×25 mL) and H₂O (25 mL). The organic phase was dried over MgSO₄, filtered and evaporated. Purification by flash column chromatography provided a white oil which was crystallized from ethanol/water. (0.180 g, 47%)

1HNMR (400 MHz, DMSO-d6) δ ppm 1.22 (s, 9H) 1.32-1.48 (m, 4H) 1.51-1.74 (m, 2H) 2.15 (s, 3H) 3.35-3.45 (m, 1H) 3.58-3.80 (m, 5H) 3.82-3.95 (m, 1H) 4.12 (t, J=4.70 Hz, 2H) 4.29 (d, J=5.64 Hz, 2H) 4.55-4.67 (m, 1H) 5.08 (s, 2H) 5.15 (s, 2H) 5.86-5.98 (m, 2H) 6.25 (s, 1H) 6.60 (d, J=7.79 Hz, 1H) 6.64 (s, 1H) 6.79 (dd, J=8.19, 2.28 Hz, 1H) 6.89-6.97 (m, 1H) 6.98-7.14 (m, 5H) 7.21 (t., J=7.92 Hz, 1H) 7.30-7.40 (m, 1H) 7.45 (dd, J=8.32, 5.91 Hz, 1H) 8.35 (s, 1H). HRMS (m/z) 768.3633. M+H, C₄₃H₅₀FN₅O₇ requires 768.3773.

Using the method described above, the following compound was prepared.

### Example 35

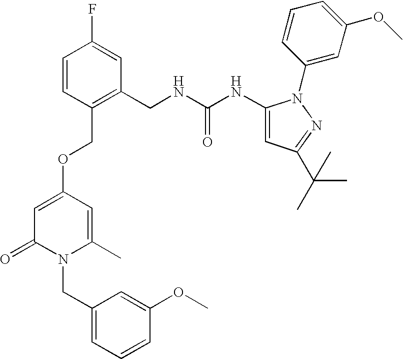

### 1-[3-tert-butyl-1-(3-methoxyphenyl)-1H-pyrazol-5-yl]-3-[5-fluoro-2-({[1-(3-methoxybenzyl)-6-methyl-2-oxo-1,2-dihydropyridin-4-yl]oxy}methyl)benzyl]urea

1HNMR (400 MHz, DMSO-d6) δ ppm 1.22 (s, 9H) 2.15 (s, 3H) 3.68 (s, 3H) 3.74 (s, 3H) 4.29 (d, J=5.64 Hz, 2H) 5.09 (s, 2H) 5.15 (s, 2H) 5.93 (d, J=5.37 Hz, 2H), 6.25 (s, 1H) 6.60 (d, J=7.79 Hz, 1H) 6.64 (s, 1H) 6.79 (dd, J=8.06, 1.88 Hz, 1H) 6.87-6.96 (m, 1H) 7.00-7.14 (m, 5H) 7.21 (t, J=7.92 Hz, 1H) 7.35 (t, J=8.06 Hz, 1H) 7.45 (dd, J=8.19, 5.17 Hz, 1H) 8.34 (s, 1H) HRMS (m/z) 654.3036. M+H, C₃₇H₄₀FN₅O₅ requires 654.3091.

### Example 36

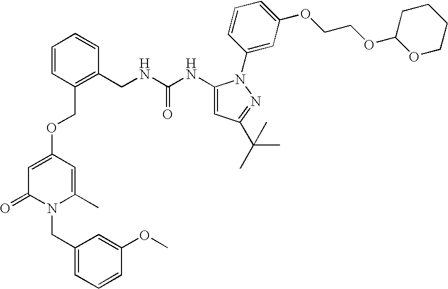

### 1-(3-tert-butyl-1-{3-[2-(tetrahydro-2H-pyran-2-yloxy)ethoxy]phenyl}-1H-pyrazol-5-yl)-3-[2-({[1-(3-methoxybenzyl)-6-methyl-2-oxo-1,2-dihydropyridin-4-yl]oxy}methyl)benzyl]urea

### Step 1: Synthesis of 2-(2-((1-(3-methoxybenzyl)-6-methyl-2-oxo-1,2-dihydropyridin-4-yloxy)methyl)benzyl)isoindoline-1,3-dione

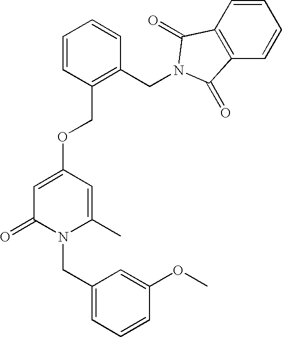

1-(3-methoxybenzyl)-4-hydroxy-6-methylpyridin-2(1H)-one (3.4 g, 13.9 mmol) was dissolved in DMF (100 mL). 2-(2-(chloromethyl)benzyl)isoindoline-1,3-dione (2.54 g, 13.9 mmol), and DBU (2.07 mL, 2.1 g, 13.9 mmol) were added. The reaction mixture was stirred at 80° C. for six hours and at room temperature overnight. Water (100 mL) was added to the reaction mixture. It was then transferred to a separatory funnel and extracted with ethyl acetate (2×100 mL). The combined organic phases were washed with brine, dried over MgSO₄, filtered, and evaporated. Purification by flash column chromatography resulted in a white solid (1.06 g, 31%). ¹HNMR (400 MHz, DMSO-d6) δ ppm 2.09 (s, 3H) 3.69 (s, 3H) 4.84 (s, 2H) 5.15 (s, 2H) 5.19 (s, 2H) 5.68 (d, J=2.15 Hz, 1H) 5.94 (d, J=2.95 Hz, 1H) 6.57-6.68 (m, 2H) 6.80 (dd, J=7.92, 2.28 Hz, 1H) 7.23 (t, J=7.92 Hz, 1H) 7.25-7.35 (m, 3H) 7.40-7.49 (m, 1H) 7.71-7.85 (m, 4H) HRMS (m/z) 495.1883. M+H, C₃₀H₂₆N₂O₅ requires 495.1920.

### Step 2: Synthesis of 1-(3-tert-butyl-1-{3-[2-(tetrahydro-2H-pyran-2-yloxy)ethoxy]-phenyl}-1H-pyrazol-5-yl)-3-[2-({[1-(3-methoxybenzyl)-6-methyl-2-oxo-1,2-dihydro-pyridin-4-yl]oxy}methyl)benzyl]urea

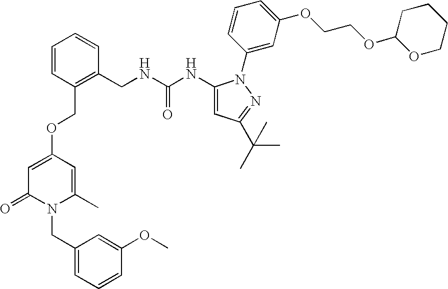

2-(2-((1-(3-methoxybenzyl)-6-methyl-2-oxo-1,2-dihydropyridin-4-yloxy)methyl)benzyl)isoindoline-1,3-dione (1.06 g, 2.14 mmol) was slurried in ethanol (50 mL). Hydrazine monohydrate (0.500 mL, 0.515 g, 10.2 mmol) was added. The reaction was stirred at 70° C. for three hours and at room temperature overnight. The resulting solid was filtered and washed with ethanol. The mother liquor was evaporated and 0.25 g (0.686 mmol) of the resulting yellow oil was dissolved in THF (15 mL). Triethylamine (0.2 mL, 0.145 g, 14 mmol) was added, followed by phenyl 3-tert-butyl-1-(3-(2-(tetrahydro-2H-pyran-2-yloxy)ethoxy)phenyl)-1H-pyrazol-5-ylcarbamate (0.329 g, 0.686 mmol). The reaction was stirred overnight at room temperature. The mixture was diluted with ethyl acetate (50 mL), and extracted with 2.5 N NaOH (2×25 mL) and H₂O (25 mL). The organic phase was dried over MgSO₄, filtered and evaporated. Purification by flash column chromatography provided a white oil which was crystallized from ethanol/ether. (0.090 g, 18%) 1HNMR (400 MHz, DMSO-d6) δ ppm 1.22 (s, 9H) 1.34-1.51 (m, 4H) 1.53-1.73 (m, 2H) 2.15 (s, 3H) 3.35-3.47 (m, 1H) 3.61-3.79 (m, 5H) 3.82-3.95 (m, 1H) 4.12 (s, 2H) 4.29 (d, J=5.10 Hz, 2H) 4.61 (s, 1H) 5.06-5.25 (m, 4H) 5.93 (s, 2H) 6.24 (s, 1H) 6.53-6.71 (m, 2H) 6.79 (d, J=6.44 Hz, 1H) 6.89-6.99 (m, 2H) 7.00-7.09 (m, 2H) 7.16-7.46 (m, 6H) 8.27 (s, 1H). HRMS (m/z) 750.3819. M+H, C₄₃H₅₁N₅O₇ requires 750.3867.

### Example 37

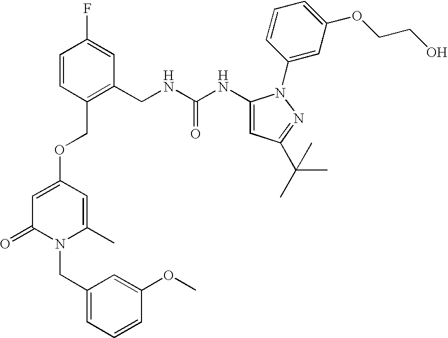

### 1-{3-tert-butyl-1-[3-(2-hydroxyethoxy)phenyl]-1H-pyrazol-5-yl}-3-[5-fluoro-2-({[1-(3-methoxybenzyl)-6-methyl-2-oxo-1,2-dihydropyridin-4-yl]oxy}methyl)benzyl]urea

1-(3-tert-butyl-1-{3-[2-(tetrahydro-2H-pyran-2-yloxy)ethoxy]phenyl}-1H-pyrazol-5-yl)-3-[5-fluoro-2-({[1-(3-methoxybenzyl)-6-methyl-2-oxo-1,2-dihydropyridin-4-yl]-oxy}methyl)benzyl]urea (0.150 g, 0.195 mmol) was suspended in methanol (15 mL). p-Toluenesulfonic acid monohydrate (20 mg, 0.105 mmol) was added (all solids dissolved upon addition). The reaction was stirred at room temperature for 2 hours. It was diluted with ethyl acetate (50 mL), and extracted with NaHCO₃ (aq) (25 mL) and brine (25 mL). The organic phase was dried over MgSO₄, filtered, and evaporated. The resulting white solid was washed with ether. (0.099 g, 74%)

1HNMR (400 MHz, DMSO-d6) δ ppm 1.22 (s, 9H) 2.15 (s, 3H) 3.61-3.75 (m, 5H) 3.98 (t, J=4.97 Hz, 2H) 4.29 (d, J=5.64 Hz, 2H) 5.08 (s, 2H) 5.15 (s, 2H) 5.84-5.97 (m, 2H) 6.25 (s, 1H) 6.60 (d, J=7.79 Hz, 1H) 6.64 (s, 1H) 6.79 (dd, J=7.92, 2.28 Hz, 1H) 6.89-6.96 (m, 1H) 7.00-7.13 (m, 5H) 7.21 (t., J=7.92 Hz, 1H) 7.29-7.37 (m, 1H) 7.45 (dd, J=8.32, 5.91 Hz, 1H) 8.34 (s, 1H).

HRMS (m/z) 684.3095. M+H, C₃₈H₄₂FN₅O₆ requires 684.3197.

Using the method described above, the following two compounds were prepared.

### Example 38

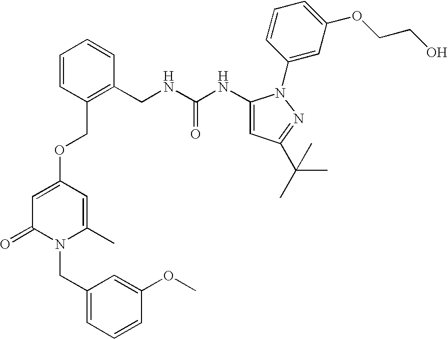

### 1-{3-tert-butyl-1-[3-(2-hydroxyethoxy)phenyl]-1H-pyrazol-5-yl}-3-[2-({[1-(3-methoxybenzyl)-6-methyl-2-oxo-1,2-dihydropyridin-4-yl]oxy}methyl)benzyl]urea

1HNMR (400 MHz, DMSO-d6) δ ppm 1.22 (s, 9H) 2.15 (s, 3H) 3.58-3.75 (m, 5H) 3.98 (t, J=4.97 Hz, 2H) 4.29 (d, J=5.37 Hz, 2H) 5.11 (s, 2H) 5.15 (s, 2H) 5.94 (s, 2H) 6.26 (s, 1H) 6.60 (d, J=7.52 Hz, 1H) 6.64 (s, 1H) 6.79 (dd, J=8.06, 2.15 Hz, 1H) 6.90-7.01 (m, 2H) 7.02-7.07 (m, 2H) 7.15-7.47 (m, 6H) 8.30 (s, 1H)

HRMS (m/z) 666.3235. M+H, C₃₈H₄₃N₅O₆ requires 666.3292.

### Example 39

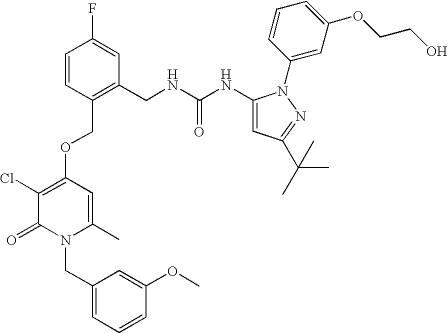

### 1-{3-tert-butyl-1-[3-(2-hydroxyethoxy)phenyl]-1-pyrazol-5-yl}-3-[2-({[3-chloro-1-(3-methoxybenzyl)-6-methyl-2-oxo-1,2-dihydropyridin-4-yl]oxy}methyl)-5-fluorobenzyl]urea

1HNMR (400 MHz, DMSO-d6) δ ppm 1.21 (s, 9H) 2.26 (s, 3H) 3.62-3.72 (m, 5H) 3.98 (t, J=4.97 Hz, 2H) 4.33 (d, J=5.91 Hz, 2H) 5.24 (s, 2H) 5.29 (s, 2H) 6.24 (s, 1H) 6.53 (s, 1H) 6.57 (d, J=7.79 Hz, 1H) 6.64 (d, J=1.88 Hz, 1H) 6.81 (dd, J=8.06, 2.42 Hz, 1H) 6.91 (dd, J=8.46, 1.48 Hz, 1H) 6.99-7.15 (m, 5H) 7.21 (t., J=8.06 Hz, 1H) 7.33 (t, J=8.06 Hz, 1H) 7.44 (d, J=8.32 Hz, 1H) 7.50 (dd, J=8.46, 5.77 Hz, 1H) 8.35 (s, 1H).

HRMS (m/z) 718.2787. M+H, C₃₈H₄₁ClFN₅O₆ requires 718.2808.

### Example 40

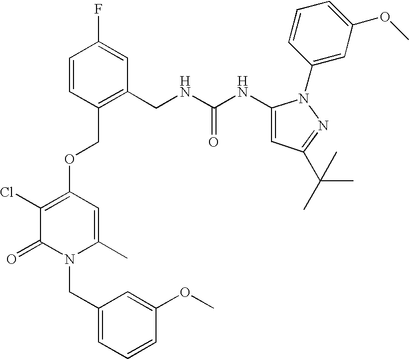

### 1-[3-tert-butyl-1-(3-methoxyphenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea

### Step 1: Synthesis of 1-(3-methoxybenzyl)-4-hydroxy-3-iodo-6-methylpyridin-2(1H)-one

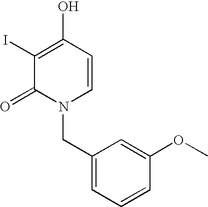

1-(3-methoxybenzyl)-4-hydroxy-6-methylpyridin-2(1H)-one (14.0 g, 57.0 mmol) was slurried in acetonitrile (300 mL). The mixture was cooled to 0° C. in an ice-water bath. N-iodosuccinimide (12.82 g, 57.0 mmol) was added. The reaction stirred at 0° C. for two hours. The solid was filtered and washed with acetonitrile. 20.3 g, (96%) 1HNMR (400 MHz, DMSO-d6) δ ppm 2.16 (s, 3H) 3.68 (s, 3H) 5.21 (s, 2H) 5.92 (s, 1H) 6.56 (d, J=7.52 Hz, 1H) 6.61 (d, J=2.15 Hz, 1H) 6.79 (dd, J=8.06, 2.42 Hz, 1H) 7.21 (t, J=7.92 Hz, 1H) HRMS (m/z) 372.0047. M+H, C₁₄H₁₄INO₃ requires 372.0091.

### Step 2: Synthesis of 1-(3-methoxybenzyl)-3-chloro-4-hydroxy-6-methylpyridin-2(1H)-one

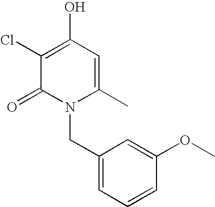

1-(3-methoxybenzyl)-4-hydroxy-3-iodo-6-methylpyridin-2(1H)-one (12.0 g, 32.0 mmol) was slurried in DMF (100 mL). The reaction was heated at 70° C. (solids dissolved upon heating) Lithium chloride (10.9 g, 259 mmol) was added in portions over a period of thirty minutes. A slight exotherm was observed. The reaction was stirred at 90° C. for five hours and at room temperature overnight. Water was added to the reaction mixture, which resulted in the formation of a precipitate. The solid was filtered and washed with H₂O. The product was dried under vacuum. 8.7 g, (97%)

1HNMR (400 MHz, DMSO-d6) δ ppm 2.16 (s, 3H) 3.68 (s, 3H) 5.19 (s, 2H) 5.97 (s, 1H) 6.57 (d, J=7.79 Hz, 1H) 6.62 (d, J=2.15 Hz, 1H) 6.80 (dd, J=8.06, 2.42 Hz, 1H) 7.21 (t, J=7.92 Hz, 1H)

HRMS (m/z) 280.0722. M+H, C₁₄H₁₄ClNO₃ requires 280.0735.

### Step 3: Synthesis of tert-butyl 2-((1-(3-methoxybenzyl)-3-chloro-6-methyl-2-oxo-1,2-dihydropyridin-4-yloxy)methyl)-5-fluorobenzylcarbamate

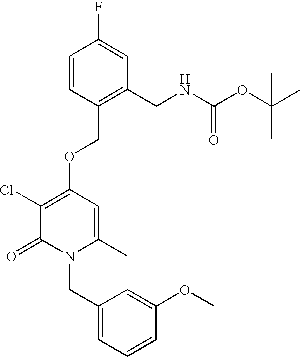

1-(3-methoxybenzyl)-3-chloro-4-hydroxy-6-methylpyridin-2(1H)-one (1.8 g, 6.52 mmol) was dissolved in DMF (50 mL). tert-Butyl 2-(bromomethyl)-5-fluorobenzylcarbamate (2.28 g, 7.17 mmol) and potassium carbonate (0.900 g, 6.52 mmol) were added. The reaction was stirred overnight at room temperature. It was diluted with ethyl acetate (100 mL), and extracted with H₂O (50 mL) and brine (50 mL). The organic phase was dried over MgSO₄, filtered, and evaporated. The crude reaction mixture was purified by flash column chromatography, resulting in a white solid. (2.0 g, 59%) 1 HNMR (400 MHz, DMSO-d6) δ ppm 1.35 (s, 9H) 2.28 (s, 3H) 3.69 (s, 3H) 4.22 (d, J=5.91 Hz, 2H) 5.25 (s, 2H) 5.28 (s, 2H) 6.51-6.61 (m, 2H) 6.65 (s, 1H) 6.81 (dd, J=8.06, 2.42 Hz, 1H) 7.00-7.16 (m, 2H) 7.21 (t, J=7.92 Hz, 1H) 7.42 (t, J=5.37 Hz, 1H) 7.50 (dd, J=8.32, 5.91 Hz, 1H)

HRMS (m/z) 517.1888 M+H, C₂₇H₃₀ClFN₂O₅ requires 517.1906.

### Step 4: Synthesis of 1-[3-tert-butyl-1-(3-methoxyphenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea

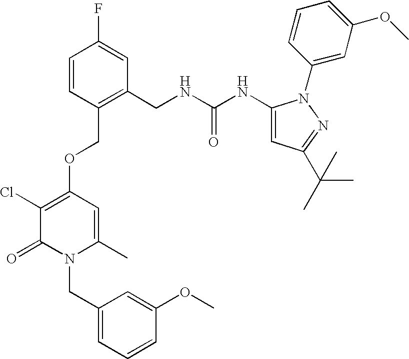

tert-Butyl 2-((1-(3-methoxybenzyl)-3-chloro-6-methyl-2-oxo-1,2-dihydropyridin-4-yloxy)methyl)-5-fluorobenzylcarbamate (0.80 g, 1.55 mmol) was dissolved in dioxane (30 mL). HCl in dioxane (3.0 mL of a 4.0 N solution) was added. The reaction was stirred overnight at room temperature. The solvent was evaporated and the resulting solid was suspended in THF (15 mL). Phenyl 3-tert-butyl-1-(3-methoxyphenyl)-1H-pyrazol-5-ylcarbamate (0.755 g, 2.1 mmol) and triethylamine (2 mL, 2.75 g, 2.72 mmol)) were added. The reaction mixture was stirred at 70° C. for two hours. The resulting precipitate was filtered and washed with THF and H₂O. (0.7 g, 65%)

1HNMR (400 MHz, DMSO-d6) δ ppm 1.22 (s, 9H) 2.25 (s, 3H) 3.67 (s, 3H) 3.74 (s, 3H) 4.35 (d, J=4.03 Hz, 2H) 5.23 (s, 2H) 5.28 (s, 2H) 6.35 (s, 1H) 6.51 (s, 1H) 6.57 (d, J=7.79 Hz, 1H) 6.62 (d, J=2.15 Hz, 1H) 6.78 (dd, J=8.06, 2.42 Hz, 1H) 6.97 (dd, J=8.06, 1.88 Hz, 1H) 7.04-7.12 (m, 4H) 7.13-7.23 (m, 2H) 7.37 (t, J=8.19 Hz, 1H) 7.45-7.54 (m 1H) 8.54 (s, 1H). HRMS (m/z) 688.2661. M+H, C₃₇H₃₉ClFN₅O₅ requires 688.2702.

Using the method described above, the following three compounds were prepared.

### Example 41

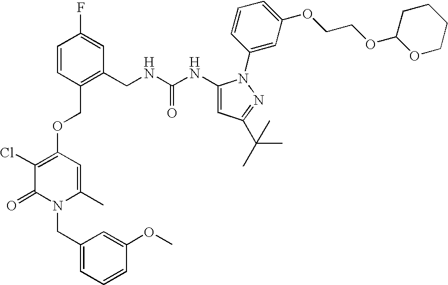

### 1-(3-tert-butyl-1-{3-[2-(tetrahydro-2H-pyran-2-yloxy)ethoxy]phenyl}-1H-pyrazol-5-yl)-3-[2-({[3-chloro-1-(3-methoxybenzyl)-6-methyl-2-oxo-1,2-dihydropyridin-4-yl]oxy}methyl)-5-fluorobenzyl]urea

1HNMR (400 MHz, DMSO-d6) δ ppm 1.21 (s, 9H) 1.35-1.50 (m, 4H) 1.54-1.74 (m, 2H) 2.26 (s, 3H) 3.33-3.45 (m, 1H) 3.61-3.77 (m, 5H) 3.82-3.92 (m, 1H) 4.12 (t, J=4.57 Hz, 2H) 4.33 (d, J=5.91 Hz, 2H) 4.61 (s, 1H) 5.24 (s, 2H) 5.29 (s, 2H) 6.24 (s, 1H) 6.53 (s, 1H) 6.57 (d, J=8.06 Hz, 1H) 6.64 (s, 1H) 6.78-6.84 (m, 1H) 6.89-6.96 (m, 1H) 7.00-7.15 (m, 5H) 7.21 (t, J=7.92 Hz, 1H) 7.33 (t, J=8.19 Hz, 1H) 7.50 (dd, J=8.46, 5.77 Hz, 1H) 8.35 (s, 1H). HRMS (m/z) 802.3390. M+H, C₄₃H₄₉ClFN₅O₇ requires 802.3377.

### Example 42

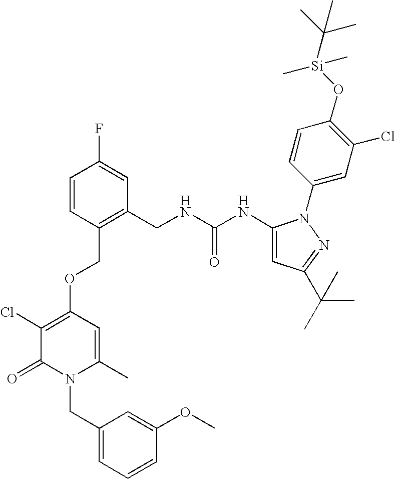

### 1-[3-tert-butyl-1-(4-{[tert-butyl(dimethyl)silyl]oxy}-3-chlorophenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea

1HNMR (400 MHz, DMSO-d6) δ ppm 0.22 (s, 6H) 0.98 (s, 9H), 1.21 (s, 9H) 2.26 (s, 3H) 3.68 (s, 3H) 4.32 (d, J=5.64 Hz, 2H) 5.24 (s, 2H) 5.29 (s, 2H) 6.21 (s, 1H) 6.54 (s, 1H) 6.57 (d, J=7.25 Hz, 1H) 6.64 (s, 1H) 6.81 (dd, J=8.19, 2.28 Hz, 1H) 7.00-7.15 (m, 4H) 7.21 (t, J=7.92 Hz, 1H) 7.31 (dd, J=8.73, 2.25 Hz, 1H) 7.45-7.59 (m, 2H) 8.36 (s, 1H). HRMS (m/z) 822.3012. M+H, C₄₂H₅₀Cl₂FN₅O₅Si requires 822.3015.

### Example 43

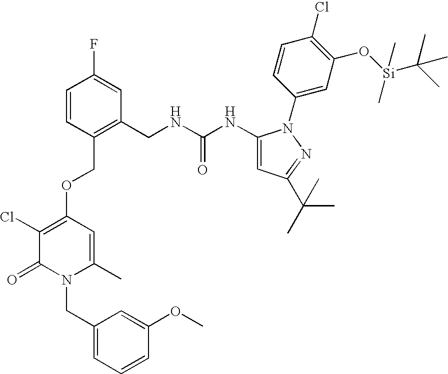

### 1-[3-tert-butyl-1-(3-{[tert-butyl(dimethyl)silyl]oxy}-4-chlorophenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea

1HNMR (400 MHz, DMSO-d6) δ ppm 0.18 (s, 6H) 0.94 (s, 9H), 1.21 (s, 9H) 2.26 (s, 3H) 3.68 (s, 3H) 4.33 (d, J=5.91 Hz, 2H) 5.24 (s, 2H) 5.29 (s, 2H) 6.22 (s, 1H) 6.54 (s, 1H) 6.58 (d, J=7.79 Hz, 1H) 6.65 (s, 1H) 6.81 (dd, J=8.32, 2.15 Hz, 1H) 7.00-7.16 (m, 5H) 7.21 (t, J=7.92 Hz, 1H) 7.44-7.55 (m, 2H) 8.37 (s, 1H). HRMS (m/z) 822.3049. M+H, C₄₂H₅₀Cl₂FN₅O₅Si requires 822.3015.

### Example 44

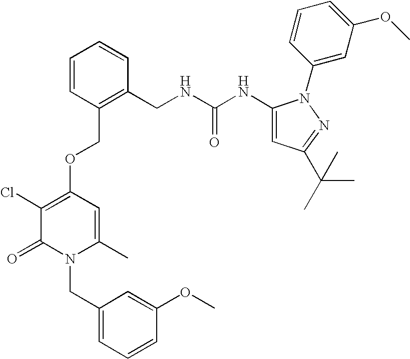

### 1-[3-tert-butyl-1-(3-methoxyphenyl)-1H-pyrazol-5-yl]-3-[2-({[3-chloro-1-(3-methoxybenzyl)-6-methyl-2-oxo-1,2-dihydropyridin-4-yl]oxy}methyl)benzyl]urea

### Step 1: Synthesis of 2-(2-((1-(3-methoxybenzyl)-3-chloro-6-methyl-2-oxo-1,2-dihydropyridin-4-yloxy)methyl)benzyl)isoindoline-1,3-dione

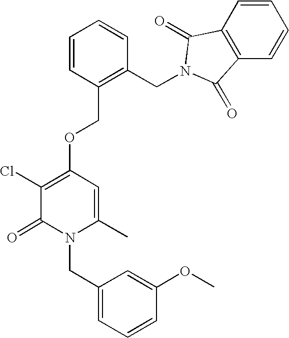

1-(3-methoxybenzyl)-3-chloro-4-hydroxy-6-methylpyridin-2(1H)-one (1.8 g, 6.52 mmol) was dissolved in DMF (50 mL). Potassium carbonate (0.927 g, 6.72 mmol) and 1-(3-methoxybenzyl)-3-chloro-4-hydroxy-6-methylpyridin-2(1H)-one (1.23 g, 6.72 mmol) were added. The reaction was stirred at 80° C. for eight hours and at room temperature overnight. Ethyl acetate (100 mL) was added. The reaction mixture was transferred to a separatory funnel and extracted with H₂O (100 mL) and brine (100 mL). The organic phase was dried over MgSO₄, filtered, and evaporated. Purification by flash column chromatography afforded a light yellow solid that was washed with ether. (1.8 g, 51%)

1HNMR (400 MHz, DMSO-d6) δ ppm 2.30 (s, 3H) 3.69 (s, 3H) 4.88 (s, 2H) 5.25 (s, 2H) 5.43 (s, 2H) 6.55-6.62 (m, 2H) 6.66 (s, 1H) 6.82 (dd, J=7.92, 2.28 Hz, 1H) 7.16-7.28 (m, 2H) 7.28-7.36 (m, 2H) 7.50 (dd, J=5.37, 3.49 Hz, 1H) 7.73-7.90 (m, 4H)

HRMS (m/z) 529.1512. M+H, C₃₀H₂₅ClN₂O₅ requires 529.1530.

### Step 2: Synthesis of 1-[3-tert-butyl-1-(3-methoxyphenyl)-1H-pyrazol-5-yl]-3-[2-({[3-chloro-1-(3-methoxybenzyl)-6-methyl-2-oxo-1,2-dihydropyridin-4-yl]oxy}methyl)benzyl]urea

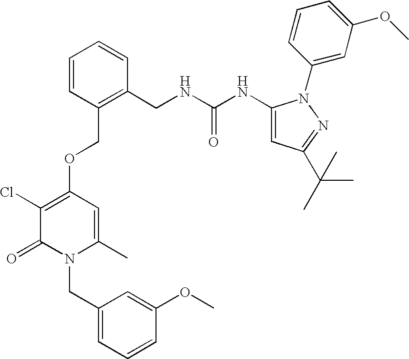

2-(2-((1-(3-methoxybenzyl)-3-chloro-6-methyl-2-oxo-1,2-dihydropyridin-4-yloxy)methyl)benzyl)isoindoline-1,3-dione. (1.0 g, 1.89 mmol) was slurried in ethanol (50 mL). Hydrazine monohydrate (0.5 mL, 0.515 g, 10.3 mmol) was added. The reaction was stirred at 70° C. overnight. The reaction was cooled to room temperature and the resulting solids were filtered and washed with ethanol. The mother liquor was evaporated and 0.45 g (1.13 mmol), of the resulting yellow oil was dissolved in THF (20 mL). Phenyl 3-tert-butyl-1-(3-methoxyphenyl)-1H-pyrazol-5-ylcarbamate (0.755 g, 2.1 mmol) and triethylamine (0.5 mL, 0.689 g, 6.8 mmol)) were added. The reaction mixture was stirred at 70° C. for four hours and overnight at room temperature. It was diluted with ethyl acetate (50 mL) then extracted with 2.5 N NaOH (25 mL) and brine (25 mL). The organic phase was dried over MgSO₄, filtered, and evaporated to. Purification by flash column chromatography afforded a white solid. (0.317 g, 42%) ¹HNMR (400 MHz, DMSO-d6) δ ppm 1.21 (s, 9H) 2.25 (s, 3H) 3.68 (s, 3H) 3.73 (s, 3H) 4.32 (d, J=5.64 Hz, 2H) 5.24 (s, 2H) 5.32 (s, 2H) 6.23 (s, 1H) 6.52 (s, 1H) 6.58 (d, J=7.52, 1H) 6.65 (s, 1H) 6.81 (dd, J=8.06, 2.42 Hz, 1H) 6.87-6.94 (m, 1H) 6.97-7.06 (m, 3H) 7.21 (t, J=7.92 Hz 1H) 7.24-7.38 (m, 4H) 7.43-7.47 (m, 1H) 8.26 (s, 1H) HRMS (m/z) 670.2775. M+H, C₃₇H₄₀ClN₅O₅ requires 670.2796.

### Example 45

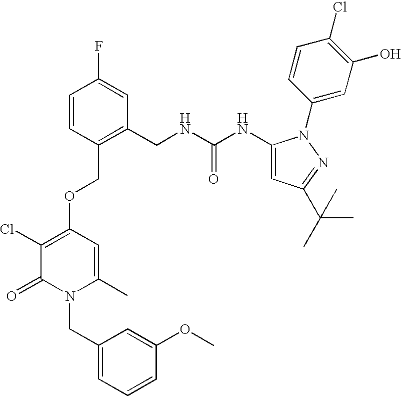

### 1-[3-tert-butyl-(4-chloro-3-hydroxyphenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea

1-[3-tert-butyl-1-(3-{[tert-butyl(dimethyl)silyl]oxy}-4-chlorophenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea (0.122 g, 0.148 mmol) was dissolved in THF (6 mL). Tetrabutylammonium formate (0.3 mL, 0.3 mmol, 3.0 M in THF) was added. The reaction mixture was stirred overnight at room temperature. It was diluted with ethyl acetate (50 mL) and extracted with brine (2×25 mL). The organic phase was dried over MgSO₄, filtered, and evaporated. The crude product was filtered through a plug of silica gel with ethyl acetate to afford a white solid. (0.065 g, 62%)

1HNMR (400 MHz, DMSO-d6) δ ppm 1.21 (s, 9H) 2.26 (s, 3H) 3.69 (s, 3H) 4.34 (d, J=5.64 Hz, 2H) 5.24 (s, 2H) 5.29 (s, 2H) 6.22 (s, 1H) 6.54 (s, 1H) 6.58 (d, J=7.79 Hz, 1H) 6.64 (s, 1H) 6.81 (dd, J=8.06, 2.15 Hz, 1H) 6.91 (dd, J=8.59, 2.42 Hz, 1H) 6.98-7.15 (m, 4H) 7.22 (t, J=7.92 Hz, 1H) 7.35 (d, J=8.59 Hz, 1H) 7.50 (dd, J=8.32, 5.91 Hz, 1H) 8.36 (s, 1H) HRMS (m/z) 708.2151. M+H, C₃₆H₃₆Cl₂FN₅O₅ requires 708.2150.

Using the method described above, the following compound was prepared.

### Example 46

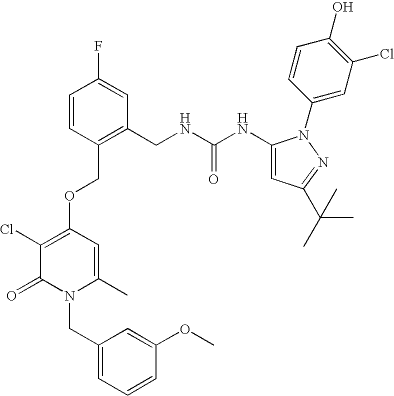

### 1-[3-tert-butyl-1-(3-chloro-4-hydroxyphenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea

1HNMR (400 MHz, DMSO-d6) δ ppm 1.20 (s, 9H) 2.26 (s, 3H) 3.69 (s, 3H) 4.33 (d, J=5.91 Hz, 2H) 5.24 (s, 2H) 5.30 (s, 2H) 6.19 (s, 1H) 6.51-6.60 (m, 2H) 6.64 (s, 1H) 6.81 (dd, J=8.73, 2.82 Hz, 1H) 6.97-7.16 (m, 5H) 7.18-7.25 (m, 2H) 7.39 (d, J=2.42 Hz, 1H) 7.47-7.58 (m, 1H) 8.26 (s, 1H). HRMS (m/z) 708.2161. M+H, C₃₆H₃₆Cl₂FN₅O₅ requires 708.2150.

### Example 47

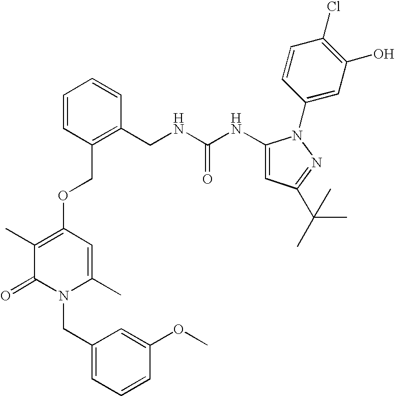

### 1-[3-tert-butyl-1-(4-chloro-3-hydroxyphenyl)-1H-pyrazol-5-yl]-3-[2-({[1-(3-methoxybenzyl)-3,6-dimethyl-2-oxo-1,2-dihydropyridin-4-yl]oxy}methyl)benzyl]urea

### Step 1: Synthesis of 2-(2-((1-(3-methoxybenzyl)-3-iodo-6-methyl-2-oxo-1,2-dihydropyridin-4-yloxy)methyl)benzyl)isoindoline-1,3-dione

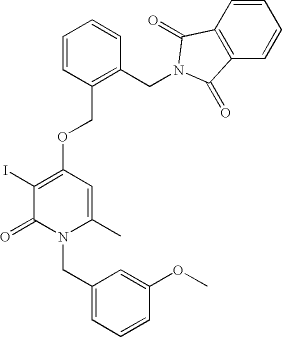

1-(3-methoxybenzyl)-4-hydroxy-3-iodo-6-methylpyridin-2(1H)-one (1.3 g, 3.5 mmol) was dissolved in DMF (50 mL). Potassium carbonate (0.483 g, 3.50 mmol) and 1-(3-methoxybenzyl)-3-chloro-4-hydroxy-6-methylpyridin-2(1H)-one (0.643 g, 3.50 mmol) were added. The reaction was stirred at 80° C. for eight hours and at room temperature overnight. Water was added until the reaction turned cloudy. The resulting tan precipitate was filtered. It was recrystallized from ethanol/water. (1.1 g, 51%). ¹HNMR (400 MHz, DMSO-d6) δ ppm 2.30 (s, 3H) 3.69 (s, 3H) 4.89 (s, 2H) 5.28 (s, 2H) 5.44 (s, 2H) 6.48 (s, 1H) 6.60 (d, J=7.52 Hz, 1H) 6.64 (d, J=2.15 Hz, 1H) 6.81 (dd, J=8.32, 2.15 Hz, 1H) 7.14-7.26 (m, 2H) 7.28-7.35 (m, 2H) 7.56 (dd, J=6.31, 2.82 Hz, 1H) 7.71-7.92 (m, 4H). HRMS (m/z) 621.0752. M+H, C₃₀H₂₅IN₂O₅ requires 621.0886.

### Step 2: Synthesis of 2-(2-((1-(3-methoxybenzyl)-3,6-dimethyl-2-oxo-1,2-dihydropyridin-4-yloxy)methyl)benzyl)isoindoline-1,3-dione

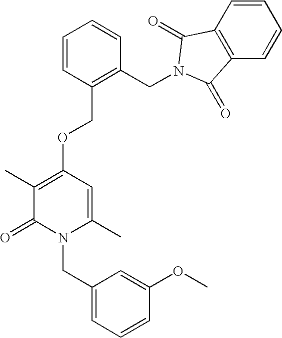

2-(2-((1-(3-methoxybenzyl)-3-iodo-6-methyl-2-oxo-1,2-dihydropyridin-4-yloxy)methyl)benzyl)isoindoline-1,3-dione (1.00 g, 1.61 mmol) was dissolved in DMF (50 mL). Tetramethyltin (0.446 mL, 0.576 g, 3.22 mmol), lithium chloride (0.239 g, 5.63 mmol) and [1,1′-Bis(diphenylphosphino)ferrocene]-dichloropalladium(II) complex with CH₂Cl₂ (0.131 g, 0.161 mmol) were added. The reaction was stirred overnight at 70° C. It was cooled to room temperature, and ethyl acetate (100 mL) was added. The reaction was extracted with H₂O (50 mL) and brine (50 mL). The organic phase was dried over MgSO₄, filtered, and evaporated. The compound was purified by flash column chromatography. It was further purified by recrystallization from ethanol/water. (0.630 g, 77%). ¹HNMR (400 MHz, DMSO-d6) δ ppm 1.73 (s, 3H) 2.25 (s, 3H) 3.68 (s, 3H) 4.88 (s, 2H) 5.21 (s, 2H) 5.31 (s, 2H) 6.39 (s, 1H) 6.53-6.69 (m, 2H) 6.80 (dd, J=8.06, 2.42 Hz, 1H) 7.16-7.27 (m, 2H) 7.30 (dd, J=5.91, 3.22 Hz, 2H) 7.47 (dd, J=5.24, 3.89 Hz, 1H) 7.70-7.96 (m, 4H). HRMS (m/z) 509.2081. M+H, C₃₁H₂₈N₂O₅ requires 509.2071.

### Step 3: Synthesis of 1-[3-tert-butyl-1-(4-chloro-3-hydroxyphenyl)-1H-pyrazol-5-yl]-3-[2-({[1-(3-methoxybenzyl)-3,6-dimethyl-2-oxo-1,2-dihydropyridin-4-yl]oxy}methyl)benzyl]urea

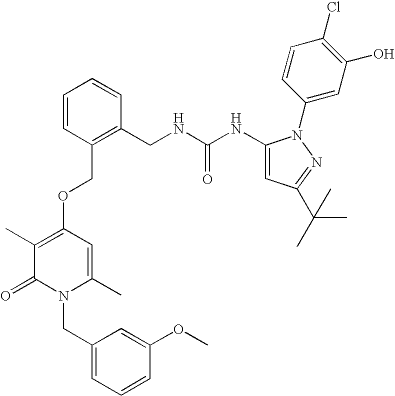

2-(2-((1-(3-methoxybenzyl)-3,6-dimethyl-2-oxo-1,2-dihydropyridin-4-yloxy)methyl)benzyl)isoindoline-1,3-dione (0.59 g, 1.16 mmol) was slurried in ethanol (25 mL). Hydrazine monohydrate (0.3 mL, 0.309 g, 5.17 mmol) was added and the reaction was stirred at 70° C. for four hours, and at room temperature overnight. The solids were filtered and washed with ethanol. The mother liquor was evaporated and 0.2 g (0.528 mmol) of the resulting yellow oil was dissolved in THF (30 mL). Triethylamine (0.5 mL, 0.363 g, 3.59 mmol) and phenyl 3-tert-butyl-1-(3-(tert-butyldimethylsilyloxy)-4-chlorophenyl)-1H-pyrazol-5-ylcarbamate (0.275 g, 0.550 mmol) were added. The reaction was stirred at room temperature for four hours. Tetrabutylammonium fluoride (0.6 mL, 0.6 mmol, of a 1.0 M in THF) was added, and the reaction stirred overnight at room temperature. The reaction mixture was diluted with ethyl acetate (50 mL) and transferred to a separatory funnel. It was extracted with H₂O (25 mL) and brine (25 mL). The organic phase was dried over MgSO₄, filtered, and evaporated. The compound was purified by flash column chromatography. A white solid was isolated. (0.066 g, 19%). ¹HNMR (400 MHz, DMSO-d6) δ ppm 120 (s, 9H) 1.86 (s, 3H) 2.20 (s, 3H) 3.68 (s, 3H) 4.32 (d, J=5.64 Hz, 2H) 5.20 (s, 4H) 6.22 (s, 1H) 6.34 (s, 1H) 6.57 (d, J=7.52 Hz, 1H) 6.62 (s, 1H) 6.78 (dd, J=8.32, 2.15 Hz, 1H) 6.89 (dd, J=8.59, 2.42 Hz, 1H) 6.92-6.99 (m, 1H) 7.08 (d, J=2.42 Hz 1H) 7.16-7.33 (m, 4H) 7.36 (d, J=8.59 Hz, 1H) 7.43 (d, J=6.71 Hz, 1H) 8.27 (s, 1H). MS (m/z) 670.2799. M+H, C₃₇H₄₀ClN₅O₅ requires 670.2791.

### Example 48

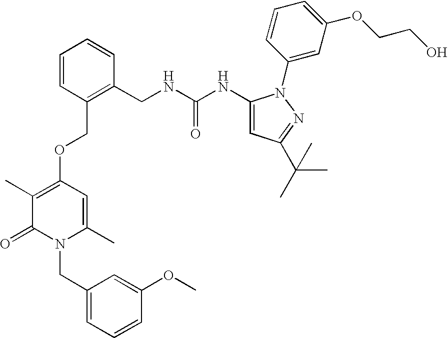

### 1-{3-tert-butyl-1-[3-(2-hydroxyethoxy)phenyl]-1H-pyrazol-5-yl}-3-[2-({[1-(3-methoxybenzyl)-3,6-dimethyl-2-oxo-1,2-dihydropyridin-4-yl]oxy}methyl)benzyl]urea

2-(2-((1-(3-methoxybenzyl)-3,6-dimethyl-2-oxo-1,2-dihydropyridin-4-yloxy)methyl)benzyl)isoindoline-1,3-dione (0.59 g, 1.16 mmol) was slurried in ethanol (25 mL). Hydrazine monohydrate (0.3 mL, 0.309 g, 5.17 mmol) was added and the reaction was stirred at 70° C. for four hours, and at room temperature overnight. The solids were filtered and washed with ethanol. The mother liquor was evaporated and 0.2 g (0.528 mmol) of the resulting yellow oil was dissolved in THF (30 mL). Triethylamine (0.5 mL, 0.363 g, 3.59 mmol) and phenyl 3-tert-butyl-1-(3-(2-(tetrahydro-2H-pyran-2-yloxy)ethoxy)phenyl)-1H-pyrazol-5-ylcarbamate (0.260 g, 0.550 mmol) were added. The reaction was stirred at room temperature for four hours. It was diluted with ethyl acetate (25 mL) and extracted with 2.5 N NaOH (25 mL) and H₂O (2×25 mL). The organic phase was dried over MgSO₄, filtered, and evaporated. The resulting oil was dissolved in methanol (15 mL). p-Toluenesulfonic acid monohydrate (20 mg, 0.105 mmol) was added was added, and the reaction stirred overnight at room temperature. The reaction mixture was diluted with ethyl acetate (50 mL) and transferred to a separatory funnel. It was extracted with H₂O (25 mL) and brine (25 mL). The organic phase was dried over MgSO₄, filtered, and evaporated. The compound was purified by flash column chromatography. A white solid was isolated. (0.063 g, 18%). ¹HNMR (400 MHz, DMSO-d6) δ ppm 1.21 (s, 9H) 1.86 (s, 3H) 2.20 (s, 3H) 3.63-3.73 (m, 5H) 3.93-4.02 (m, 2H) 4.32 (d, J=5.64 Hz, 2H) 5.20 (s, 4H) 6.23 (s, 1H) 6.33 (s, 1H) 6.57 (d, J=7.79 Hz, 1H) 6.62 (s, 1H) 6.78 (dd, J=8.32, 2.42 Hz, 1H) 6.92 (dd, J=8.19, 1.48 Hz, 1H) 6.98-7.04 (m, 3H) 7.20 (t., J=8.06 Hz, 1H) 7.23-7.37 (m, 4H) 7.42 (d, J=7.52 Hz, 1H) 8.26 (s, 1H). HRMS (m/z) 680.3434. M+H, C₃₉H₄₅N₅O₆ requires 680.3443.

### Example 49

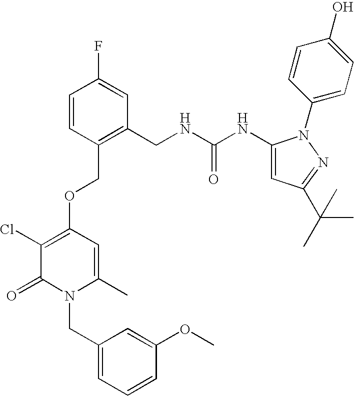

### 1-[3-tert-butyl-1-(4-hydroxyphenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea

tert-Butyl 2-((1-(3-methoxybenzyl)-3-chloro-6-methyl-2-oxo-1,2-dihydropyridin-4-yloxy)methyl)-5-fluorobenzylcarbamate (0.263 g, 0.51 mmol) was dissolved in dioxane (10 mL). HCl in dioxane (1.0 mL of a 4.0 N solution) was added. The reaction was stirred overnight at room temperature. The solvent was evaporated and the resulting solid was suspended in THF (15 mL). Phenyl 3-tert-butyl-1-(4-(tert-butyldimethylsilyloxy)phenyl)-1H-pyrazol-5-ylcarbamate (0.248 g, 0.53 mmol) and triethylamine (2 mL, 2.75 g, 2.72 mmol)) were added. The reaction mixture was stirred at room temperature for three hours. Tetrabutylammonium fluoride (0.6 mL, 0.6 mmol, of a 1.0 M in THF) was added, and the reaction stirred overnight at room temperature. The reaction mixture was diluted with ethyl acetate (50 mL) and transferred to a separatory funnel. It was extracted with H₂O (25 mL) and brine (25 mL). The organic phase was dried over MgSO₄, filtered, and evaporated. The compound was purified by flash column chromatography. A white solid was isolated. (0.210 g, 61%). ¹HNMR (400 MHz, DMSO-d6) δ ppm 1.20 (s, 9H) 2.26 (s, 3H) 3.69 (s, 3H) 4.33 (d, J=6.18 Hz, 2H) 5.24 (s, 2H) 5.30 (s, 2H) 6.21 (s, 1H) 6.52-6.61 (m, 2H) 6.64 (s, 1H) 6.77-6.87 (m, 3H) 7.01-7.16 (m, 3H) 7.22 (t, J=7.92 Hz, 3H) 7.50 (dd, J=8.46, 5.77 Hz, 1H) 8.20 (s, 1H). HRMS (m/z) 674.2526. M+H, C₃₆H₃₇ClFN₅O₅ requires 674.2540.

Using the method described above, the following compound was prepared.

### Example 50

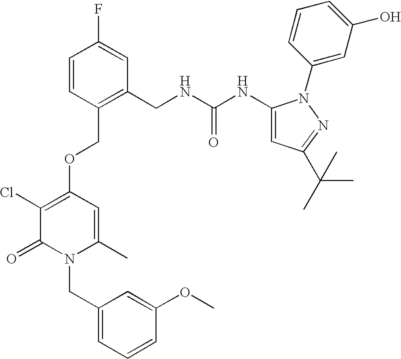

### 1-[3-tert-butyl-1-(3-hydroxyphenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea

¹HNMR (400 MHz, DMSO-d6) δ ppm 1.21 (s, 9H) 2.26 (s, 3H) 3.69 (s, 3H) 4.34 (d, J=5.64 Hz, 2H) 5.24 (s, 2H) 5.30 (s, 2H) 6.22 (s, 1H) 6.54 (s, 1H) 6.58 (s, J=7.79 Hz, 1H) 6.64 (s, 1H) 6.71-6.76 (m, 1H) 6.81 (dd, J=8.19, 2.28 Hz, 1H) 6.85-6.91 (m, 2H) 7.03-7.16 (m, 3H) 7.22 (t, J=7.92 Hz, 2H) 7.50 (dd, J=8.46, 5.77 Hz, 1H) 8.32 (s, 1H). HRMS (m/z) 674.2550. M+H, C₃₆H₃₇ClFN₅O₅ requires 674.2540.

### Example 51

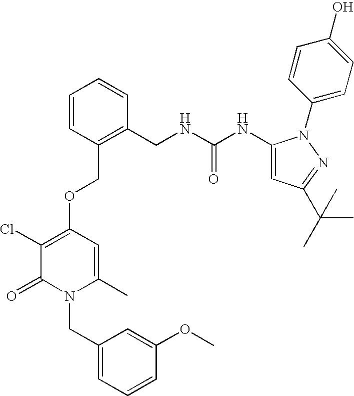

### 1-[3-tert-butyl-1-(4-hydroxyphenyl)-1H-pyrazol-5-yl]-3-[2-({[3-chloro-1-(3-methoxybenzyl)-6-methyl-2-oxo-1,2-dihydropyridin-4-yl]oxy}methyl)benzyl]urea

2-(2-((1-(3-methoxybenzyl)-3-chloro-6-methyl-2-oxo-1,2-dihydropyridin-4-yloxy)methyl)benzyl)isoindoline-1,3-dione. (0.385 g, 0.728 mmol) was slurried in ethanol (25 mL). Hydrazine monohydrate (0.25 mL, 0.257 g, 5.13 mmol) was added. The reaction was stirred at 70° C. overnight. The reaction was cooled to room temperature and the resulting solids were filtered and washed with ethanol. The mother liquor was evaporated and the resulting yellow oil was dissolved in THF (15 mL). Phenyl 3-tert-butyl-1-(4-(tert-butyldimethylsilyloxy)phenyl)-1H-pyrazol-5-ylcarbamate (0.0372 g, 0.799 mmol) and triethylamine (0.5 mL, 0.689 g, 6.8 mmol)) were added. The reaction mixture was stirred at room temperature for four hours. Tetrabutylammonium fluoride (0.8 mL, 0.8 mmol, of a 1.0 M in THF) was added, and the reaction stirred overnight at room temperature. The organic phase was dried over MgSO₄, filtered, and evaporated. Purification by flash column chromatography afforded a white solid. (0.160 g, 34%). ¹HNMR (400 MHz, DMSO-d6) δ ppm 1.20 (s, 9H) 2.25 (s, 3H) 3.68 (s, 3H) 4.32 (d, J=4.83 Hz, 2H) 5.24 (s, 2H) 5.32 (s, 2H) 6.24 (s, 1H) 6.53 (s, 1H) 6.57 (d, J=8.06 Hz, 1H) 6.64 (s, 1H) 6.82 (t, J=8.46 Hz, 3H) 7.01 (s, 1H) 7.14-7.38 (m, 6H) 7.45 (d, J=6.98 Hz, 1H) 8.19 (s, 1H). HRMS (m/z) 656.2629. M+H, C₃₆H₃₈ClN₅O₅ requires 656.2634.

Using the method described above, the following two compounds were prepared.

### Example 52

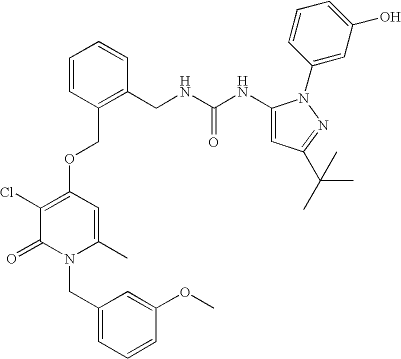

### 1-[3-tert-butyl-1-(3-hydroxyphenyl)-1H-pyrazol-5-yl]-3-[2-({[3-chloro-1-(3-methoxybenzyl)-6-methyl-2-oxo-1,2-dihydropyridin-4-yl]oxy}methyl)benzyl]urea

¹HNMR (400 MHz, DMSO-d6) δ ppm 1.20 (s, 9H) 2.26 (s, 3H) 3.68 (s, 3H) 4.33 (d, J=5.64 Hz, 2H) 5.24 (s, 2H) 5.32 (s, 2H) 6.23 (s, 1H) 6.53 (s, 1H) 6.58 (d, J=7.79 Hz, 1H) 6.65 (s, 1H) 6.72-6.77 (m, 1H) 6.81 (dd, J=8.19, 2.28 Hz, 1H) 6.84-6.89 (m, 2H) 7.02 (t, J=5.91 Hz 1H) 7.16-7.39 (m, 5H) 7.42-7.47 (m, 1H) 8.26 (s, 1H). HRMS (m/z) 656.2655. M+H, C₃₆H₃₈ClN₅O₅ requires 656.2634.

### Example 53

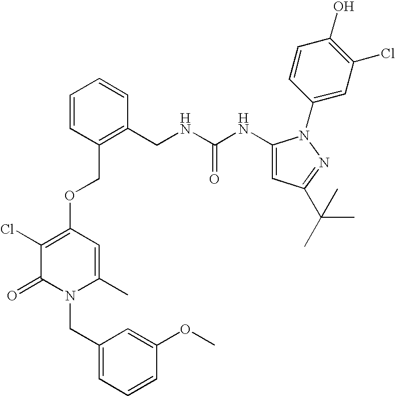

### 1-[3-tert-butyl-1-(3-chloro-4-hydroxyphenyl)-1H-pyrazol-5-yl]-3-[2-({[3-chloro-1-(3-methoxybenzyl)-6-methyl-2-oxo-1,2-dihydropyridin-4-yl]oxy}methyl)benzyl]urea

¹HNMR (400 MHz, DMSO-d6) δ ppm 1.23 (s, 9H) 2.29 (s, 3H) 3.72 (s, 3H) 4.35 (d, J=5.13 Hz, 2H) 5.27 (s, 2H) 5.35 (s, 2H) 6.21 (s, 1H) 6.54 (s, 1H) 6.62 (d, J=7.32 Hz, 1H) 6.68 (s, 1H) 6.84 (d, J=7.32 Hz, 1H) 6.90-7.08 (m, 2H) 7.18-7.57 (m, 7H) 8.15 (s, 1H), 10.42 (s. 1H). HRMS (m/z) 690.2554. M+H, C₃₆H₃₇Cl₂N₅O₅ requires 690.2245.

### Example 54

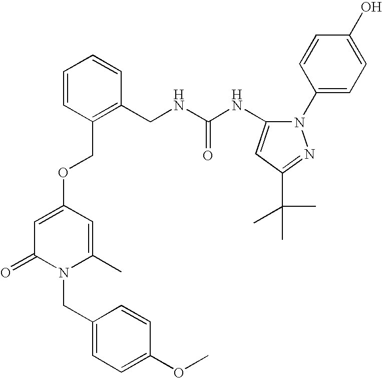

### Synthesis of 1-[3-tert-butyl-1-(4-hydroxyphenyl)-1H-pyrazol-5-yl]-3-[2-({[1-(4-methoxybenzyl)-6-methyl-2-oxo-1,2-dihydropyridin-4-yl]oxy}methyl)benzyl]urea

### General Procedure B

### Step 1: Preparation of 4-hydroxy-1-(4-methoxybenzyl)-6-methylpyridin-2(1H)-one

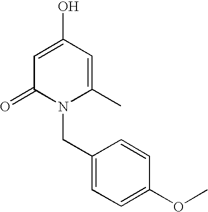

4-Methoxy benzylamine (10 g, 72.9 mmol) was added to a suspension of 4-hydroxy-6-methyl-2-pyrone (9.2 g, 72.9 mmol) in water (200 mL). The reaction mixture was heated at reflux for 8 hours. The product precipitates during the course of the reaction. The reaction mixture was cooled to room temperature and the solids were filtered, and washed sequentially with water and diethyl ether. The title compound was isolated as a white solid (15.5 g). ¹H NMR (400 MHz, DMSO-d₆) δ ppm 2.14 (s, 3H) 3.68 (s, 3H) 5.07 (s, 2H) 5.55 (d, J=2.69 Hz, 1H) 5.74 (d, J=1.88 Hz, 1H) 6.80-6.88 (m, 2H) 7.02 (d, J=8.59 Hz, 2H) 10.43 (s, 1H).

### Step 2: Preparation of 2-[2-({[1-(4-methoxybenzyl)-6-methyl-2-oxo-1,2-dihydropyridin-4-yl]oxy}methyl)benzyl]-1H-isoindole-1,3(2H)-dione

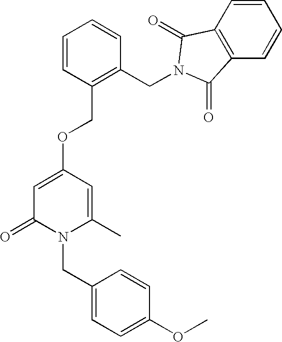

A 250 mL round bottomed flask was charged with 4-hydroxy-1-(4-methoxybenzyl)-6-methylpyridin-2(1H)-one (3 g, 12.3 mmol) and N,N′-dimethylformamide (75 mL). Potassium carbonate (1.86 g, 13.5 mmol) and 2-[2-(chloromethyl)benzyl]-1H-isoindole-1,3(2H)-dione (3.8 g, 13.5 mmol) were added and the reaction mixture was stirred under nitrogen at 55° C. overnight. The reaction was quenched with saturated aqueous NaHCO₃ and extracted with ethyl acetate. The extract was washed with brine, dried over Na₂SO₄, decanted and concentrated in vacuo. The title compound solidified under vacuum (2.23 g). ¹H NMR (400 MHz, DMSO-d6) δ ppm 2.11 (s, 3H) 3.69 (s, 3H) 4.83 (s, 2H) 5.10 (s, 2H) 5.18 (s, 2H) 5.65 (d, J=2.15 Hz, 1H) 5.92 (d, J=2.69 Hz, 1H) 6.87 (d, J=8.86 Hz, 2H) 7.05 (d, J=8.59 Hz, 2H) 7.23-7.34 (m, 3H) 7.40-7.49 (m, 1H) 7.67-7.95 (m, 4H).). LC/MS, tᵣ=3.43 minutes (5 to 95% acetonitrile/water over 6 minutes at 1 ml/min with detection 254 nm, at 50° C.). ES-MS m/z 495 (M+H).

### Step 3: Preparation of 4-{[2-(aminomethyl)benzyl]oxy}-1-(4-methoxybenzyl)-6-methylpyridin-2(1H)-one

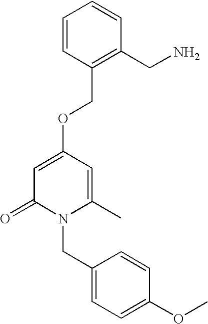

Hydrazine hydrate (2.3 mL, 47.5 mmol) was added to a suspension of 2-[2-({[1-(4-methoxybenzyl)-6-methyl-2-oxo-1,2-dihydropyridin-4-yl]oxy}methyl)benzyl]-1H-isoindole-1,3(2H)-dione (4.7 g, 9.5 mmol) in methanol (200 mL). The solution became clear as the reaction stirred at room temperature. An additional 2 mL of hydrazine hydrate was added after 3 h and the reaction mixture stirred an additional 12 h. The solids were removed by filtration and were suspended in ethanol. Concentrated HCl (5 mL) was added and the solution was heated for 5 minutes and then cooled back to room temperature. The phthalhydrazide solids were removed by filtration. The filtrate was concentrated, diluted with water and brought to pH 10 with 2.5N NaOH. The product was extracted into ethyl acetate which was then washed with brine. The organic layer was concentrated in vacuo to give the title compound (1.8 g) that was used without further purification. ¹H NMR (400 MHz, DMSO-d₆) δ ppm 1.96 (s, 3H) 3.69 (s, 3H) 5.13 (s, 6H) 5.88-5.94 (m, J=2.28, 2.28 Hz, 2H) 6.84-6.89 (m, 2H) 7.02-7.08 (m, 2H) 7.23 (dd, J=7.52, 1.34 Hz, 1H) 7.28-7.34 (m, 1H) 7.36 (d, J=7.52 Hz, 1H) 7.44 (d, J=7.52 Hz, 1H). LC/MS, tᵣ=2.24 minutes (5 to 95% acetonitrile/water over 6 minutes at 1 ml/min with detection 254 nm, at 50° C.). ES-MS m/z 365 (M+H).

### Step 4: Preparation of 1-[3-tert-butyl-1-(4-hydroxyphenyl)-1H-pyrazol-5-yl]-3-[2-({[1-(4-methoxybenzyl)-6-methyl-2-oxo-1,2-dihydropyridin-4-yl]oxy}methyl)benzyl]urea

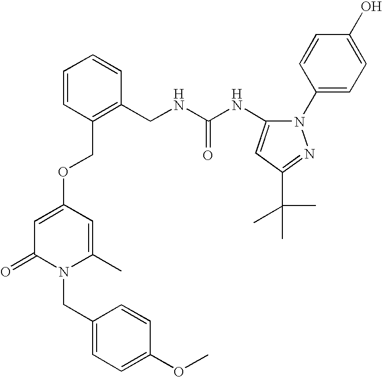

Cold phosgene (20% in toluene, 2.5 mL, 4.8 mmol) was added to a 0° C. solution of 4-{[2-(aminomethyl)benzyl]oxy}-1-(4-methoxybenzyl)-6-methylpyridin-2(1H)-one (0.3 g, 0.8 mmol) in methylene chloride (20 mL). Saturated aqueous NaHCO₃ (30 mL) was added and the reaction mixture was stirred at 0° C. for 20 minutes. The layers were separated and the organic layer was concentrated in vacuo. The residue was suspended in THF (50 mL) and a solution of 4-(5-amino-3-tert-butyl-1H-pyrazol-1-yl)phenol (0.18 g, 0.8 mmol) was added. The reaction mixture was warmed to room temperature and was stirred under nitrogen overnight. The reaction mixture was concentrated in vacuo. Solids were precipitated with acetonitrile/diethyl ether and discarded. The filtrate was concentrated and was purified on silica, eluting with 30:7 to 0:100 hexanes:ethyl acetate in a 20 minute gradient. The title compound was isolated as a white solid (100 mg). ¹H NMR (400 MHz, DMSO-d₆) δ ppm 1.20 (s, 9H) 2.16 (s, 3H) 3.69 (s, 3H) 4.27 (d, J=5.64 Hz, 2H) 5.10 (s, 3H) 5.85-5.97 (m, 2H) 6.18 (s, 1H) 6.79-6.83 (m, 2H) 6.83-6.87 (m, 2H) 6.87-6.91 (m, 1H) 7.04 (d, J=8.86 Hz, 2H) 7.15-7.21 (m, 2H) 7.22-7.35 (m, 4H) 7.37-7.42 (m, 1H) 8.05 (s, 1H) 9.68 (s, 1H).

### Example 55

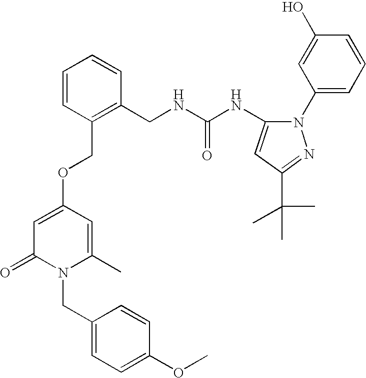

### 1-[3-tert-butyl-1-(3-hydroxyphenyl)-1H-pyrazol-5-yl]-3-[2-({[1-(4-methoxybenzyl)-6-methyl-2-oxo-1,2-dihydropyridin-4-yl]oxy}methyl)benzyl]urea

This compound was prepared using General Procedure B with 3-(5-amino-3-tert-butyl-1H-pyrazol-1-yl)phenol. ¹H NMR (400 MHz, DMSO-d₆) δ ppm 1.21 (s, 9H) 2.16 (s, 3H) 3.69 (s, 3H) 4.29 (d, J=5.91 Hz, 2H) 5.10 (s, 4H) 5.86-5.96 (m, 2H) 6.22 (s, 1H) 6.70-6.76 (m, 1H) 6.82-6.88 (m, 3H) 6.93 (t, J=5.64 Hz, 1H) 7.04 (d, J=8.59 Hz, 1H) 7.18-7.35 (m, 6H) 7.39 (d, J=6.98 Hz, 1H) 8.22 (s, 1H) 9.72 (s, 1H). LC/MS, tᵣ=3.38 minutes (5 to 95% acetonitrile/water over 6 minutes at 1 ml/min with detection 254 nm, at 50° C.). ES-MS m/z 622 (M+H).

### Example 56

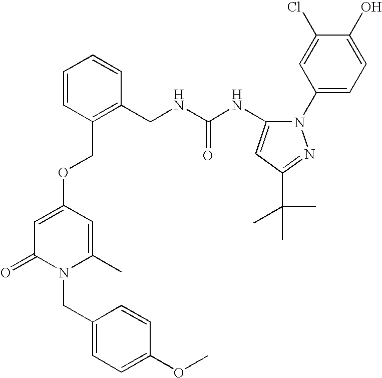

### Synthesis of 1-[3-tert-butyl-1-(3-chloro-4-hydroxyphenyl)-1H-pyrazol-5-yl]-3-[2-({[1-(4-methoxybenzyl)-6-methyl-2-oxo-1,2-dihydropyridin-4-yl]oxy}methyl)benzyl]urea

This compound was prepared using General Procedure B with 4-(5-amino-3-tert-butyl-1H-pyrazol-1-yl)-2-chlorophenol. ¹H NMR (400 MHz, DMSO-d₆) δ ppm 1.20 (s, 9H) 2.16 (s, 3H) 3.69 (s, 3H) 4.27 (d, J=5.64 Hz, 2H) 5.09 (s, 4H) 5.85-5.94 (m, 2H) 6.19 (s, 1H) 6.79-6.90 (m, 3H) 6.98-7.07 (m, 3H) 7.15-7.34 (m, 4H) 7.34-7.45 (m, 2H) 8.15 (s, 1H) 10.46 (s, 1H). LC/MS, tᵣ=3.56 minutes (5 to 95% acetonitrile/water over 6 minutes at 1 ml/min with detection 254 nm, at 50° C.). ES-MS m/z 656 (M+H).

### Example 57

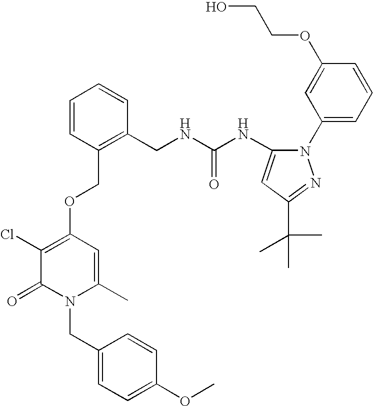

### Synthesis of 1-{3-tert-butyl-1-[3-(2-hydroxyethoxy)phenyl]-1H-pyrazol-5-yl}-3-[2-({[3-chloro-1-(4-methoxybenzyl)-6-methyl-2-oxo-1,2-dihydropyridin-4-yl]oxy}methyl)benzyl]urea

### General Procedure C

### Step 1: Preparation of 4-hydroxy-3-iodo-1-(4-methoxybenzyl)-6-methylpyridin-2(1H)-one

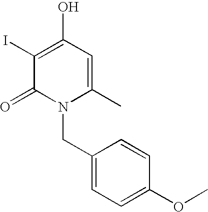

N-Iodosuccinimide (10 g, 45 mmol) was added to a 0° C. suspension of -hydroxy-1-(4-methoxybenzyl)-6-methylpyridin-2(1H)-one (10 g, 41 mmol) in acetonitrile (100 mL). The reaction mixture was warmed to room temperature and stirred overnight. The reaction mixture was filtered and the solids were washed sequentially with acetonitrile and diethyl ether. The title compound was isolated as a white solid (9 g). ¹H NMR (400 MHz, DMSO-d₆) δ ppm 2.18 (s, 3H) 3.68 (s, 3H) 5.16 (s, 2H) 5.89 (s, 1H) 6.77-6.94 (m, 2H) 7.02 (d, J=8.86 Hz, 2H) 11.32 (s, 1H).). LC/MS, tᵣ=2.54minutes (5 to 95% acetonitrile/water over 6 minutes at 1 ml/min with detection 254 nm, at 50° C.). ES-MS m/z 372 (M+H).

### Step 2: 3-chloro-4-hydroxy-1-(4-methoxybenzyl)-6-methylpyridin-2(1H)-one

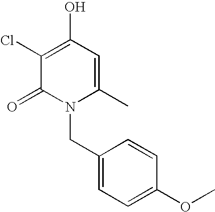

Lithium chloride (0.91 g, 21.6 mmol) was added to a solution of Preparation of 4-hydroxy-3-iodo-1-(4-methoxybenzyl)-6-methylpyridin-2(1H)-one (1 g, 2.7 mmol) in N,N′-dimethylformamide (10 mL). The reaction mixture was heated at 90° C. for 24 h. After cooling to room temperature, the solution was diluted with water. Solids were filtered and washed sequentially with water and diethyl ether. The title compound was isolated as a white solid (0.62 g). ¹H NMR (400 MHz, DMSO-d₆) δ ppm 2.18 (s, 3H) 3.68 (s, 3H) 5.14 (s, 2H) 5.94 (s, 1H) 6.79-6.94 (m, 2H) 7.02 (d, J=8.59 Hz, 2H) 11.17 (s, 1H). LC/MS, tᵣ=2.3 minutes (5 to 95% acetonitrile/water over 6 minutes at 1 ml/min with detection 254 nm, at 50° C.). ES-MS m/z 280 (M+H).

### Step 3: Preparation of 2-[2-({[3-chloro-1-(4-methoxybenzyl)-6-methyl-2-oxo-1,2-dihydropyridin-4-yl]oxy}methyl)benzyl]-1H-isoindole-1,3(2H)-dione

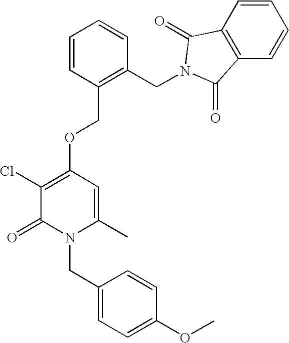

A 100 mL round bottomed flask was charged with 3-chloro-4-hydroxy-1-(4-methoxybenzyl)-6-methylpyridin-2(1H)-one (3 g, 10.7 mmol) and N,N′-dimethylformamide (50 mL). Potassium carbonate (1.6 g, 11.8 mmol) and 2-[2-(chloromethyl)benzyl]-1H-isoindole-1,3(2H)-dione (3.4 g, 11.8 mmol) were added and the reaction mixture was stirred under nitrogen at 60° C. overnight. The reaction was quenched with saturated aqueous NaHCO₃ and extracted with ethyl acetate. The extract was washed with brine, dried over Na₂SO₄, decanted and concentrated in vacuo. The title compound solidified under vacuum and was used without further purification (5.2 g). ¹H NMR (400 MHz, DMSO-d₆) δ ppm 2.32 (s, 3H) 3.69 (s, 3H) 4.88 (s, 2H) 5.20 (s, 2H) 5.42 (s, 2H) 6.56 (s, 1H) 6.77-6.95 (m, 2H) 7.06 (d, J=8.86 Hz, 2H) 7.17-7.40 (m, 3H) 7.50 (dd, J=5.50, 3.63 Hz, 1H) 7.73-7.97 (m, 4H). LC/MS, tᵣ=5.1 minutes (5 to 95% acetonitrile/water over 6 minutes at 1 ml/min with detection 254 nm, at 50° C.). ES-MS m/z 529 (M+H).

### Step 4: Preparation of 4-{[2-(aminomethyl)benzyl]oxy}-3-chloro-1-(4-methoxybenzyl)-6-methylpyridin-2(1H)-one

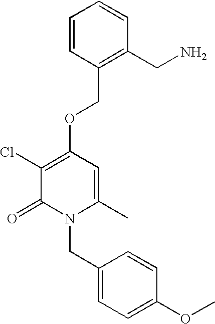

Hydrazine hydrate (2.3 mL, 47.5 mmol) was added to a suspension of 2-[2-({[3-chloro-1-(4-methoxybenzyl)-6-methyl-2-oxo-1,2-dihydropyridin-4-yl]oxy}methyl)benzyl]-1H-isoindole-1,3(2H)-dione (5 g, 9.5 mmol) in methanol (100 mL). The solution became clear as the reaction stirred at room temperature. An additional 4 mL of hydrazine hydrate was added after 3 h and the reaction mixture stirred an additional 12 h. The phthalhydrazide solids were removed by filtration and the filtrated was concentrated. The residue was suspended in ethanol. Concentrated HCl (5 mL) was added and the solution was heated for 5 minutes and then cooled back to room temperature. The title compound was isolated as the HCl salt and used without further purification. LC/MS, tᵣ=2.31 minutes (5 to 95% acetonitrile/water over 6 minutes at 1 ml/min with detection 254 nm, at 50° C.). ES-MS m/z 399 (M+H).

### Step 5: Preparation of 1-(3-tert-butyl-1-{3-[2-(tetrahydro-2H-pyran-2-yloxy)ethoxy]phenyl}-1H-pyrazol-5-yl)-3-[2-({[3-chloro-1-(4-methoxybenzyl)-6-methyl-2-oxo-1,2-dihydropyridin-4-yl]oxy}methyl)benzyl]urea

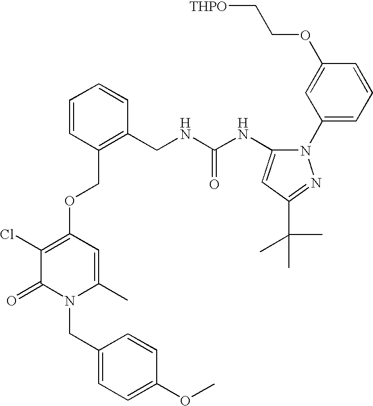

Phenyl chloroformate (0.22 mL, 1.8 mmol) was added dropwise to a 0° C. solution of 3-tert-butyl-1 {3-[2-(tetrahydro-2H-pyran-2-yloxy)ethoxy]phenyl}-1H-pyrazol-5-amine

(0.6 g, 1.67 mmol), pyridine (0.2 mL, 2.2 mmol) and THF (20 mL) The reaction mixture was stirred at 0° C. for 15 minutes and then at room temperature for 2 hours. The reaction was diluted with ethyl acetate and washed twice with water. The organic layer was dried over Na₂SO₄, decanted and concentrated in vacuo. The resulting orange oil was dissolved in THF (50 mL). To this solution was added 4-{[2-(aminomethyl)benzyl]oxy}-3-chloro-1-(4-methoxybenzyl)-6-methylpyridin-2(1H)-one (0.6 g, 0.8 mmol), triethylamine (1 mL) in THF (50 mL). The reaction mixture was heated at 60° C. for 1 hour and then was stirred at room temperature overnight. The reaction mixture was diluted with ethyl acetate and washed with 1 N NaOH. The organic layer was dried over Na₂SO₄, decanted and concentrated in vacuo. The residue was purified on silica, eluting with 100% ethyl acetate. The title compound was isolated as a white solid (295 mg). LC/MS, tᵣ=4.2 minutes (5 to 95% acetonitrile/water over 6 minutes at 1 ml/min with detection 254 nm, at 50° C.). ES-MS m/z 700(M-THP).

### Step 6: Preparation of 1-{3-tert-butyl-1-[3-(2-hydroxyethoxy)phenyl]-1H-pyrazol-5-yl}-3-[2-({[3-chloro-1-(4-methoxybenzyl)-6-methyl-2-oxo-1,2-dihydropyridin-4-yl]oxy}methyl)benzyl]urea

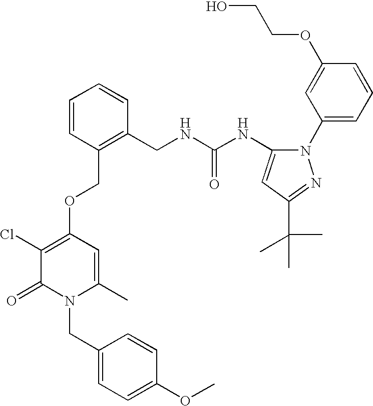

p-Toluenesulfonic acid (2 mg, 0.012 mmol) was added to a solution of 1-(3-tert-butyl-1-{3-[2-(tetrahydro-2H-pyran-2-yloxy)ethoxy]phenyl}-1H-pyrazol-5-yl)-3-[2-({[3-chloro-1-(4-methoxybenzyl)-6-methyl-2-oxo-1,2-dihydropyridin-4-yl]oxy}methyl)benzyl]urea (100 mg, 0.13 mmol) in methanol (10 mL). The reaction mixture was stirred at room temperature overnight and then was partitioned between ethyl acetate and brine. The organic layer was concentrated in vacuo and triturated with diethyl ether. The title compound was isolated as a white solid (81 mg). ¹H NMR (400 MHz, DMSO-d₆) δ ppm 1.21 (s, 9H) 2.27 (s, 3H) 3.69 (s, 3H) 3.98 (t, J=4.97 Hz, 2H) 4.32 (d, J=5.91 Hz, 2H) 4.87 (s, 1H) 5.19 (s, 2H) 5.31 (s, 2H) 6.20 (s, 1H) 6.52 (s, 1H) 6.78-6.96 (m, 3H) 6.96-7.07 (m, 4H) 7.17 (t, J=5.77 Hz, I H) 7.24-7.38 (m, 6H) 7.44 (d, J=7.25 Hz, 1H) 8.44 (s, 1H).

### Example 58

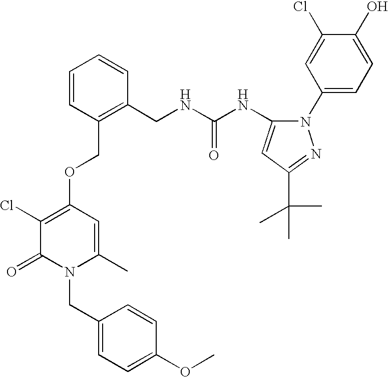

### 1-[3-tert-butyl-1-(3-chloro-4-hydroxyphenyl)-1H-pyrazol-5-yl]-3-[2-({[3-chloro-1-(4-methoxybenzyl)-6-methyl-2-oxo-1,2-dihydropyridin-4-yl]oxy}methyl)benzyl]urea

A 100 mL round bottomed flask was charged with 4-{[2-(aminomethyl)benzyl]oxy}-3-chloro-1-(4-methoxybenzyl)-6-methylpyridin-2(1H)-one (0.3 g, 0.75 mmol), triethylamine (1 mL) and THF (20 mL). Phenyl [3-tert-butyl-1-(3-chloro-4-hydroxyphenyl)-1H-pyrazol-5-yl]carbamate (0.38 g, 0.75 mmol) in THF (10 mL) was added and the reaction mixture was heated at 60° C. for 4 hours. The reaction mixture was cooled to room temperature and tetra-butyl ammonium fluoride (1M in THF, 3 mL) was added. The reaction mixture was stirred at room temperature for 4 hours and then was partitioned between ethyl acetate and brine. The organic layer was concentrated in vacuo. The residue was chromatographed on silica (100:0 to 0:100 hexanes:ethyl acetate, 40 minute gradient). The title compound was isolated as a white solid (90 mg). ¹H NMR (400 MHz, DMSO-d₆) δ ppm 1.19 (s, 9H) 2.27 (s, 3H) 3.69 (s, 3H) 4.31 (d, J=5.64 Hz, 2H) 5.19 (s, 2H) 5.30 (s, 2H) 6.18 (s, 1H) 6.50 (s, 1H) 6.86 (d, J=8.86 Hz, 1H) 6.93 (t, J=5.91 Hz, 1H) 7.02 (dd, J=13.29, 8.73 Hz, 3H) 7.18 (d, J=2.69 Hz, 1H) 7.20 (d, J=2.42 Hz, 1H) 7.22 (dd, J=7.38, 1.21 Hz, 1H) 7.24-7.36 (m, 2H) 7.38 (d, J=2.69 Hz, 1H) 7.41-7.47 (m, 1H) 8.15 (s, 1H) 10.46 (s, 1H).

### Example 59

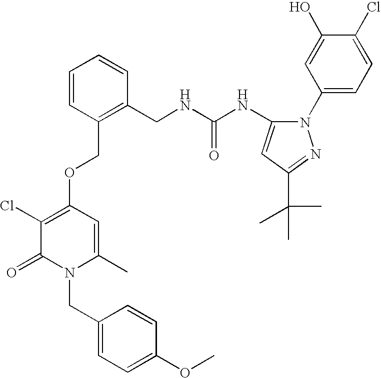

### methyl 3-{4-[(2-{[({[3-tert-butyl-1-(4-methylphenyl)-1H-pyrazol-5-yl]amino}carbonyl)amino]methyl}-4-fluorobenzyl)oxy]-2-(methylthio)-6-oxopyrimidin-1(6H)-yl}-4-methylbenzoate

A 100 mL round bottomed flask was charged with 4-{[2-(aminomethyl)benzyl]-oxy}-3-chloro-1-(4-methoxybenzyl)-6-methylpyridin-2(1H)-one (0.3 g, 0.75 mmol), triethylamine (1 mL) and THF (20 mL). Phenyl [3-tert-butyl-1-(4-chloro-3-hydroxyphenyl)-1H-pyrazol-5-yl]carbamate (0.38 g, 0.75 mmol) in THF (10 mL) was added and the reaction mixture was heated at 60° C. overnight. The reaction mixture was cooled to room temperature and tetra-butyl ammonium fluoride (1M in THF, 3 mL) was added. The reaction mixture was stirred at room temperature for 4 hours and then was partitioned between ethyl acetate and brine. The organic layer was concentrated in vacuo. The residue was chromatographed on silica (100:0 to 0:100 hexanes:ethyl acetate, 40 minute gradient). The title compound was isolated as a white solid (217 mg). ¹H NMR (400 MHz, DMSO-d₆) δ ppm 1.20 (s, 9H) 2.27 (s, 3H) 3.69 (s, 3H) 4.32 (d, J=5.91 Hz, 2H) 5.20 (s, 2H) 5.31 (s, 3H) 6.21 (s, 1H) 6.50 (s, 1H) 6.83-6.91 (m, 2H) 6.95 (t, J=5.77 Hz, 1H) 7.04 (d, J=8.59 Hz, 2H) 7.09 (d, J=2.42 Hz, 1H) 7.23-7.35 (m, 2H) 7.36 (d, J=8.32 Hz, 2H) 7.44 (dd, J=7.12, 1.48 Hz, 1H) 8.26 (s, 1H) 10.51 (s, 1H).

### Example 60

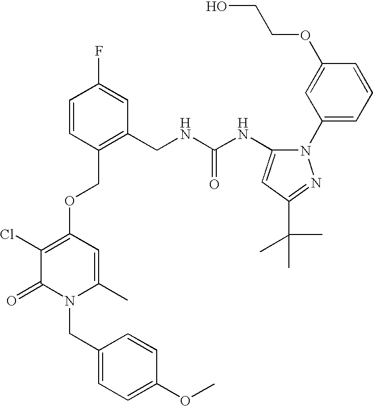

### 1-{3-tert-butyl-1-[3-(2-hydroxyethoxy)phenyl]-1H-pyrazol-5-yl}-3-[2-({[3-chloro-1-(4-methoxybenzyl)-6-methyl-2-oxo-1,2-dihydropyridin-4-yl]oxy}methyl)-5-fluorobenzyl]urea

### Step 1: Preparation of the hydrochloride salt of 4-{[2-(aminomethyl)-4-fluorobenzyl]oxy}-3-chloro-1-(4-methoxybenzyl)-6-methyl-5,6-dihydropyridin-2(1H)-one

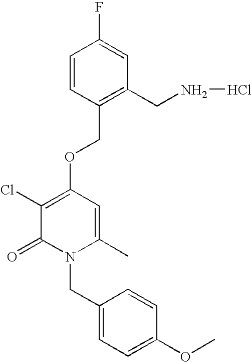

A 100 mL round bottomed flask was charged with 3-chloro-4-hydroxy-1-(4-methoxybenzyl)-6-methylpyridin-2(1H)-one (1.76 g, 6.3 mmol) and N,N′-dimethylformamide (50 mL). Potassium carbonate (0.96 g, 6.9 mmol) and tert-butyl [2-(bromomethyl)-5-fluorobenzyl]carbamate (2 g, 6.3 mmol) were added and the reaction mixture was stirred under nitrogen at 60° C. overnight. The reaction was poured into brine and was extracted with ethyl acetate. The extract was washed with brine, dried over Na₂SO₄, decanted and concentrated in vacuo. The resulting residue was dissolved in 30 mL of 4N HCl in 1,4-dioxane and heated at 60° C. for 1 hour. The reaction mixture was cooled to room temperature, poured into water and was extracted into ethyl acetate. The organic extract was concentrated in vacuo and gave 2.3 g of the title compound as the HCl salt which was used without further purification. ¹H NMR (400 MHz, DMSO-d₆) δ ppm 2.31 (s, 3H) 3.68 (s, 3H) 4.06-4.18 (m, 2H) 5.20 (s, 2H) 5.43 (s, 2H) 6.64 (s, 1H) 6.86 (d, J=8.59 Hz, 2H) 7.04 (d, J=8.59 Hz, 2H) 7.19-7.29 (m, 1H) 7.37 (s, 1H) 7.44-7.53 (m, 1H) 7.57 (dd, J=8.59, 5.91 Hz, 1H) 7.57 (dd, J=8.59, 5.91 Hz, 1H). LC/MS, tᵣ=2.3 minutes (5 to 95% acetonitrile/water over 6 minutes at 1 ml/min with detection 254 nm, at 50° C.). ES-MS m/z 417(M+H).

### Step 2: Preparation of 1-{3-tert-butyl-1-[3-(2-hydroxyethoxy)phenyl]-1H-pyrazol-5-yl}-3-[2-({[3-chloro-1-(4-methoxybenzyl)-6-methyl-2-oxo-1,2-dihydropyridin-4-yl]oxy}methyl)-5-fluorobenzyl]urea

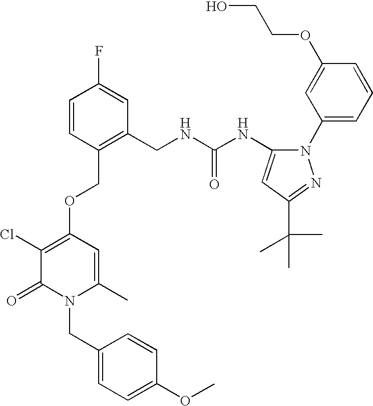

A 100 mL round bottomed flask was charged with 4-{[2-(aminomethyl)-4-fluorobenzyl]oxy}-3-chloro-1-(4-methoxybenzyl)-6-methyl-5,6-dihydropyridin-2(1H)-one (0.3 g, 0.72 mmol), triethylamine (1 mL) and THF (30 mL). The phenyl (3-tert-butyl-1-{3-[2-(tetrahydro-2H-pyran-2-yloxy)ethoxy]phenyl}-1H-pyrazol-5-yl)carbamate

(0.34 g, 0.72 mmol) was added and the reaction mixture was heated at 60° C. overnight. The reaction mixture was cooled to room temperature and diluted with ethyl acetate. The organic layer was separated and concentrated in vacuo. The residue was dissolved in methanol (50 mL) and was treated with p-toluenesulfonic acid (100 mg). The reaction mixture was stirred at room temperature for 1 hour and then was partitioned between ethyl acetate and brine. The organic layer was concentrated in vacuo. The residue was chromatographed on silica (100:0 to 0:100 hexanes:ethyl acetate, 40 minute gradient). The title compound was isolated as a white solid (125 mg). ¹H NMR (400 MHz, DMSO-d₆) δ ppm 1.21 (s, 9H) 2.28 (s, 3H) 3.69 (s, 3H) 3.98 (t, J=4.97 Hz, 2H) 4.33 (d, J=5.91 Hz, 2H) 4.79-4.88 (m, 2H) 5.20 (s, 2H) 5.28 (s, 2H) 6.23 (s, 1H) 6.50 (s, 1H) 6.83-6.89 (m, 3H) 6.91 (dd, J=7.92, 2.01 Hz, 1H) 6.99-7.08 (m, 6H) 7.08-7.15 (m, 1H) 7.32 (t, J=8.46 Hz, I H) 7.49 (dd, J=8.46, 5.77 Hz, 1H) 8.32 (s, 1H). LC/MS, tᵣ=3.39 minutes (5 to 95% acetonitrile/water over 6 minutes at 1 ml/min with detection 254 nm, at 50° C.). ES-MS m/z 720 (M+H).

### Example 61

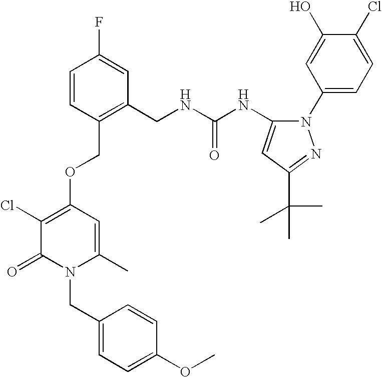

### 1-[3-tert-butyl-1-(4-chloro-3-hydroxyphenyl)-1H-pyrazol-5-yl]-3-[2-({[3-chloro-1-(4-methoxybenzyl)-6-methyl-2-oxo-1,2-dihydropyridin-4-yl]oxy}methyl)-5-fluorobenzyl]urea

A 100 mL round bottomed flask was charged with 4-{[2-(aminomethyl)-4-fluorobenzyl]oxy}-3-chloro-1-(4-methoxybenzyl)-6-methyl-5,6-dihydropyridin-2(1H)-one (0.3 g, 0.72 mmol), triethylamine (1 mL) and THF (30 mL). Phenyl [3-tert-butyl-1-(4-chloro-3-hydroxyphenyl)-1H-pyrazol-5-yl]carbamate (0.36 g, 0.72 mmol) in THF (10 mL) was added and the reaction mixture was heated at 60° C. overnight. The reaction mixture was cooled to room temperature and tetra-butyl ammonium fluoride (1M in THF, 3 mL) was added. The reaction mixture was stirred at room temperature for 4 hours and then was partitioned between ethyl acetate and brine. The organic layer was concentrated in vacuo. The residue was chromatographed on silica (100:0 to 0:100 hexanes:ethyl acetate, 40 minute gradient) The title compound was isolated as a white solid (175 mg). ¹H NMR (400 MHz, DMSO-d₆) δ ppm 1.21 (s, 9H) 2.28 (s, 3H) 3.69 (s, 3H) 4.33 (d, J=5.91 Hz, 2H) 5.20 (s, 2H) 5.28 (s, 2H) 6.22 (s, 1H) 6.51 (s, 1H) 6.84-6.88 (m, 2H) 6.90 (dd, J=8.59, 2.42 Hz, 1H) 6.99-7.07 (m, 4H) 7.07-7.15 (m, 2H) 7.35 (d, J=8.59 Hz, 1H) 7.50 (dd, J=8.46, 5.77 Hz, 1H) 8.34 (s, 1H) 10.50 (s, 1H).

### Example 62

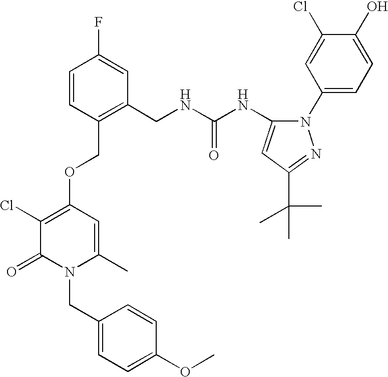

### 1-[3-tert-butyl-1-(3-chloro-4-hydroxyphenyl)-1H-pyrazol-5-yl]-3-[2-({[3-chloro-1-(4-methoxybenzyl)-6-methyl-2-oxo-1,2-dihydropyridin-4-yl]oxy}methyl)-5-fluorobenzyl]urea

A 100 mL round bottomed flask was charged with 4-{[2-(aminomethyl)-4-fluorobenzyl]oxy}-3-chloro-1-(4-methoxybenzyl)-6-methyl-5,6-dihydropyridin-2(1H)-one (0.3 g, 0.72 mmol), triethylamine (1 mL) and THF (20 mL). Phenyl [3-tert-butyl-1-(3-chloro-4-hydroxyphenyl)-1H-pyrazol-5-yl]carbamate (0.36 g, 0.72 mmol) in THF (10 mL) was added and the reaction mixture was heated at 60° C. overnight. The reaction mixture was cooled to room temperature and tetra-butyl ammonium fluoride (1M in THF, 3 mL) was added. The reaction mixture was stirred at room temperature for 4 hours and then was partitioned between ethyl acetate and brine. The organic layer was concentrated in vacuo. The residue was chromatographed on silica (100:0 to 0:100 hexanes:ethyl acetate, 40 minute gradient). The title compound was isolated as a white solid (147 mg). ¹H NMR (400 MHz, DMSO-d₆) δ ppm 1.20 (s, 9H) 2.28 (s, 3H) 3.69 (s, 3H) 4.32 (d, J=5.64 Hz, 2H) 5.19 (s, 2H) 5.28 (s, 2H) 6.18 (s, 1H) 6.51 (s, 1H) 6.86 (d, J=8.86 Hz, 2H) 6.98-7.07 (m, 5H) 7.07-7.15 (m, 1H) 7.20 (dd, J=8.73, 2.55 Hz, 1H) 7.38 (d, J=2.42 Hz, 1H) 7.49 (dd, J=8.32, 5.91 Hz, 1H) 8.23 (s, 1H) 10.45 (s, 1H).

### Intermediate 36i

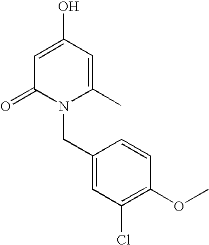

### 1-(3-Chloro-4-methoxybenzyl)-4-hydroxy-6-methylpyridin-2(1H)-one

4-Hydroxy-6-methyl-2-pyrone (19.86 g, 0.155 mol) was dissolved in water (800 mL) at 100° C. 3-Chloro-4-methoxybenzylamine (8.86 g, 0.052 mol) was added drop-wise to the above solution over 20 minutes while at 100° C. The reaction was refluxed under N₂ for 5 h. then filtered while still hot and rinsed with additional hot water. The material was air-dried to give 9.12 g (63% yield) of 1-(3-Chloro-4-methoxybenzyl)-4-hydroxy-6-methylpyridin-2(1H)-one as a sand-colored solid. ¹H NMR (300 MHz, DMSO-d₆) δ 2.17 (s, 3H), 3.80 (s, 3H), 5.09 (s, 2H), 5.58 (m, 1H), 5.77 (m, 1H), 7.03-7.17 (m, 3H), 10.48, (s, 1H); MS (ES+) for C₁₄H₁₄ClNO₃ m/z 280.2 (M+H)⁺.

### Intermediate 37i

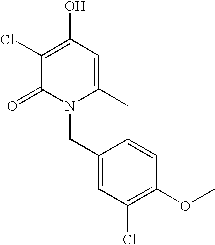

### 3-chloro-1-(3-chloro-4-methoxybenzyl)-4-hydroxy-6-methylpyridin-2(1H)-one

Solid N-chlorosuccinimide (5.09 g, 38 mmol) was added to a solution of 1-(3-Chloro-4-methoxybenzyl)-4-hydroxy-6-methylpyridin-2(1H)-one (8.9 g, 31.8 mmol) in a mixture of 1,2-dichloroethane (300 mL) and 2-propanol (200 mL) at 55° C. A second portion of N-chlorosuccinimide (0.5 g, 3.7 mmol) was added after 1 h. After ½ h, the reaction was evaporated to yellow solid. This material was triturate from methylene chloride and filtered to give 4.63 g (46% yield) of 3-chloro-1-(3-chloro-4-methoxybenzyl)-4-hydroxy-6-methylpyridin-2(1H)-one as an off-white solid. ¹H NMR (300 MHz, DMSO-d₆) δ 2.21 (s, 3H), 3.81 (s, 3H), 5.15 (s, 2H), 5.98 (s, 1H), 7.02-7.19 (m, 3H), 11.25 (s, 1H); MS (ES+) for C₁₄H₁₃Cl₂NO₃ m/z 314.18 (M+H)⁺.

### Example 63

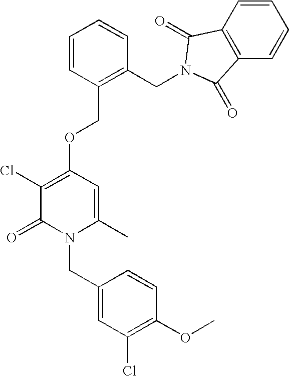

### 2-[2-({[3-chloro-1-(3-chloro-4-methoxybenzyl)-6-methyl-2-oxo-1,2-dihydropyridin-4-yl]oxy}methyl)benzyl]-1H-isoindole-1,3(2H)-dione

Potassium carbonate (1.45 g, 10.5 mmol) and 2-[2-(chloromethyl)benzyl]-1H-isoindole-1,3(2H)-dione (3 g, 10.5 mmol) were added to a room temperature solution of 3-chloro-1-(3-chloro-4-methoxybenzyl)-4-hydroxy-6-methylpyridin-2(1H)-one (3 g, 9.55 mmol) in DMF (60 mL), then heated at 55° C. under N₂ for 4 h. The reaction was concentrated to approximately ⅓ of the original volume and diluted to 400 mL total volume with water. The solid was filtered, rinsed with additional chilled water and air-dried overnight. This material was triturated with diethyl ether, filtered and air-dried to give 5.0 g (92% yield) of 2-[2-({[3-chloro-1-(3-chloro-4-methoxybenzyl)-6-methyl-2-oxo-1,2-dihydropyridin-4-yl]oxy}methyl)benzyl]-1H-isoindole-1,3(2H)-dione as a tan solid. ¹H NMR (300 MHz, DMSO-d₆) δ 2.35 (s, 3H), 3.81 (s, 3H), 4.91 (s, 2H), 5.25 (s, 2H), 5.44 (s, 2H), 5.74 (s, 1H), 6.59 (d, 1H), 7.09-7.34 (m, 5H), 7.50 (m, 1H), 7.84 (m, 4H); MS (ES+) for C₃₀H₂₄Cl₂N₂O₅ m/z 563.27 (M+H)⁺.

### Intermediate 38i

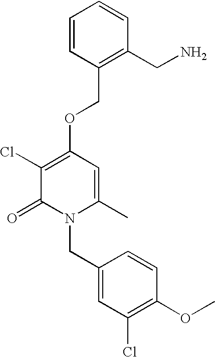

### 4-{[2-(aminomethyl)benzyl]oxy}-3-chloro-1-(3-chloro-4-methoxybenzyl)-6-methylpyridin-2(1H)-one

Hydrazine hydrate (13 mL, 268 mmol) was added to a suspension of 2-[2-({[3-chloro-1-(3-chloro-4-methoxybenzyl)-6-methyl-2-oxo-1,2-dihydropyridin-4-yl]oxy}methyl)benzyl]-1H-isoindole-1,3(2H)-dione (4.0 g, 7.09 mmol) in MeOH (260 mL) and stirred at room temperature overnight. The reaction was evaporated and partitioned between EtOAc (250 mL) and NaOH (2.5 N, 125 mL). The EtOAc layer was washed with water, separated, dried over MgSO₄ and evaporated to give 2.80 g (87% yield) of 4-{[2-(aminomethyl)benzyl]oxy}-3-chloro-1-(3-chloro-4-methoxybenzyl)-6-methylpyridin-2(1H)-one as an off-white solid. ¹H NMR (300 MHz, DMSO-d₆) δ 2.32 (s, 3H), 3.80 (s, 2H), 3.81 (s, 3H), 5.21 (s, 2H), 5.35 (s, 2H), 5.74 (s, 2H), 6.59 (s, 1H), 7.08-7.47 (m, 7H); MS (ES+) for C₂₂H₂₂Cl₂N₂O₃ m/z 433.22 (M+H)⁺.

### General Procedure D:

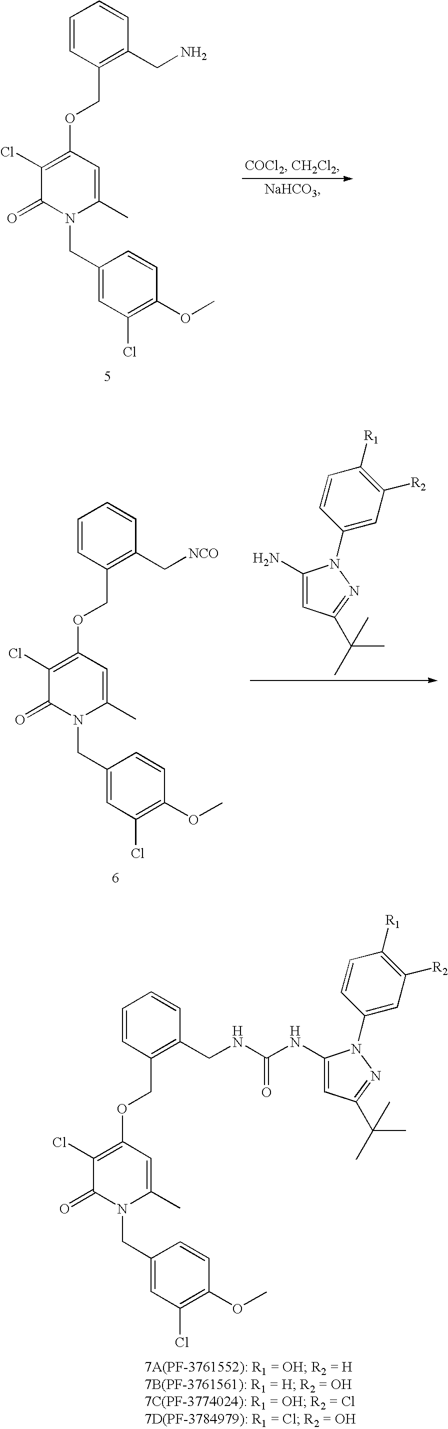

### Example 64

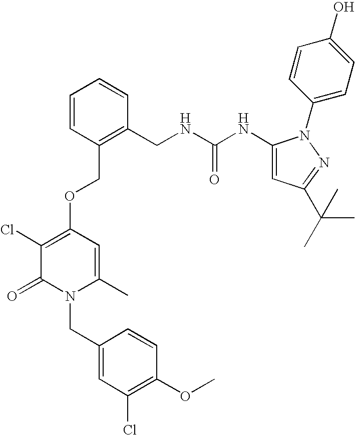

### 1-[3-tert-butyl-1-(4-hydroxyphenyl)-1H-pyrazol-5-yl]-3-[2-({[3-chloro-1-(3-chloro-4-methoxybenzyl)-6-methyl-2-oxo-1,2-dihydropyridin-4-yl]oxy}methyl)benzyl]urea

Cold phosgene (20% in toluene, 2.5 mL, 4.8 mmol) was added to a 0° C. solution of 4-{[2-(aminomethyl)benzyl]oxy}-3-chloro-1-(3-chloro-4-methoxybenzyl)-6-methylpyridin-2(1H)-one (0.346 g, 0.8 mmol) in methylene chloride (20 mL). Saturated aqueous NaHCO₃ (30 mL) was added and the reaction mixture was stirred at 0° C. for 20 minutes. The layers were separated and the organic layer was concentrated in vacuo. The residue was suspended in THF (50 mL) and a solution of 4-(5-amino-3-tert-butyl-1H-pyrazol-1-yl)phenol (0.18 g, 0.8 mmol) was added. The reaction mixture was warmed to room temperature and was stirred under nitrogen overnight. The reaction mixture was concentrated in vacuo. Solids were precipitated with acetonitrile/diethyl ether and discarded. The filtrate was concentrated and was purified by reversed phase LC. The desired fractions were combined, and 1 mL of 5% NaHCO₃ was added, and extracted with CH₂Cl₂. The organic layer was dried over Na₂SO₄ to afford the title compound as a white solid (100 mg). ¹H NMR (300 MHz, DMSO-d₆) δ 1.20 (s, 9H), 2.29 (s, 3H), 3.81 (s, 3H), 4.32 (m, 2H), 5.21 (s, 2H), 5.33 (s, 2H), 6.19 (s, 1H), 6.54 (s, 1H), 6.81 (m, 2H), 7.06-7.44 (m, 10H), 8.08 (s, 1H) 9.70 (s, 1H); MS (ES+) for C₃₆H₃₇Cl₂N₅O₅ m/z 690.48 (M+H)⁺.

### Example 65

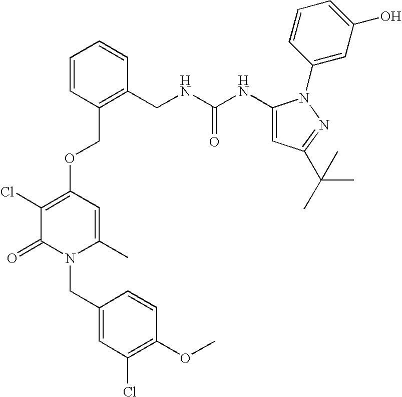

### 1-[3-tert-butyl-1-(3-hydroxyphenyl)-1H-pyrazol-5-yl]-3-[2-({[3-chloro-1-(3-chloro-4-methoxybenzyl)-6-methyl-2-oxo-1,2-dihydropyridin-4-yl]oxy}methyl)benzyl]urea

This compound was prepared using General Procedure D with 3-(5-amino-3-tert-butyl-1H-pyrazol-1-yl)phenol. ¹H NMR (300 MHz, DMSO-d₆) δ 1.22 (s, 9H), 2.30 (s, 3H), 3.81 (s, 3H), 4.33 (m, 2H), 5.21 (s, 2H), 5.33 (s, 2H), 6.22 (s, 1H), 6.54 (s, 1H), 6.87 (m, 2H), 7.06-7.44 (m, 10H), 8.24 (s, 1H) 9.73 (s, 1H); MS (ES+) for C₃₆H₃₇Cl₂N₅O₅ m/z 690.48 (M+H)⁺.

### Example 66

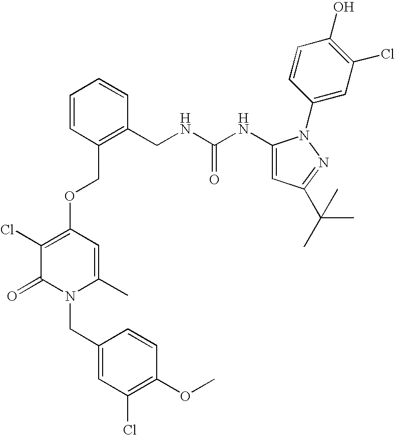

### 1-[3-tert-butyl-1-(3-chloro-4-hydroxyphenyl)-1H-pyrazol-5-yl]-3-[2-({[3-chloro-1-(3-chloro-4-methoxybenzyl)-6-methyl-2-oxo-1,2-dihydropyridin-4-yl]oxy}methyl)benzyl]urea

This compound was prepared using General Procedure B with 4-(5-amino-3-tert-butyl-1H-pyrazol-1-yl)-2-chlorophenol. ¹H NMR (300 MHz, DMSO-d₆) δ 1.21 (s, 9H), 2.29 (s, 3H), 3.81 (s, 3H), 4.32 (m, 2H), 5.21 (s, 2H), 5.33 (s, 2H), 6.19 (s, 1H), 6.54 (s, 1H), 7.00-7.47 (m, 1H), 8.17 (s, 1H); MS (ES+) for C₃₆H₃₆Cl₃N₅O₅ m/z 726.44 (M+H)⁺.

### Example 67

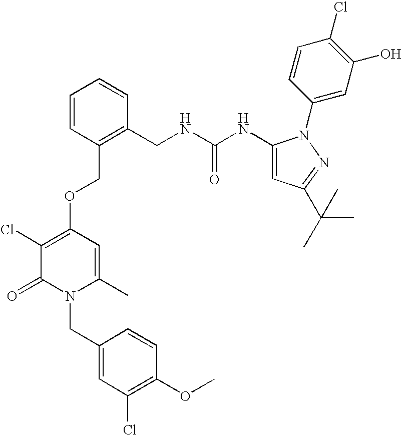

### 1-[3-tert-butyl-1-(4-chloro-3-hydroxyphenyl)-1H-pyrazol-5-yl]-3-[2-({[3-chloro-1-(3-chloro-4-methoxybenzyl)-6-methyl-2-oxo-1,2-dihydropyridin-4-yl]oxy}methyl)benzyl]urea

This compound was prepared using General Procedure B with 5-(3-tert-butyl-5-amino-1H-pyrazol-1-yl)-2-chlorophenol. ¹H NMR (300 MHz, DMSO-d₆) δ 1.22 (s, 9H), 2.30 (s, 3H), 3.81 (s, 3H), 4.33 (m, 2H), 5.21 (s, 2H), 5.33 (s, 2H), 6.23 (s, 1H), 6.54 (s, 1H), 6.92 (dd, 1H), 7.08-7.47 (m, 10H), 8.29 (s, 1H), 10.52 (s, 1H); MS (ES+) for C₃₆H₃₆Cl₃N₅O₅ m/z 726.44 (M+H)⁺.

### Example 68

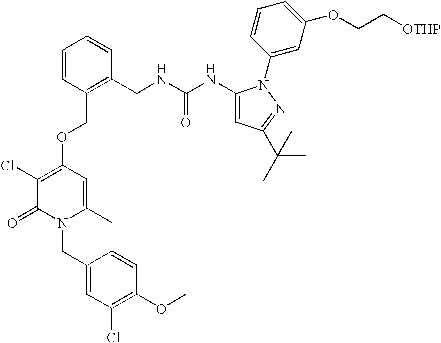

### 1-(2-((1-(3-chloro-4-methoxybenzyl)-3-chloro-1,2-dihydro-6-methyl-2-oxopyridin-4-yloxy)methyl)benzyl)-3-(1-(3-(2-(tetrahydro-2H-pyran-2-yloxy)ethoxy)phenyl)-3-tert-butyl-1H-pyrazol-5-yl)urea

Triethylamine (0.6 mL, 4.31 mmol) and phenyl 1-(3-(2-(tetrahydro-2H-pyran-2-yloxy)ethoxy)phenyl)-3-tert-butyl-1H-pyrazol-5-ylcarbamate (358.6 mg, 0.749 mmol) in THF (5 mL) was added to 4-{[2-(aminomethyl)benzyl]oxy}-3-chloro-1-(3-chloro-4-methoxybenzyl)-6-methylpyridin-2(1H)-one (0.295 g, 0.681 mmol) in THF (1 mL). After bringing the reaction up to 60° C. the reaction was stirred at room temperature overnight. The reaction was partitioned between EtOAc and 2.5 N NaOH, the EtOAc layer was separated, dried over MgSO₄ and the solvent was removed to give the appropriate urea, which was carried on without further purification. C18-HPLC t_{R}=10.67 min (85% pure), m/z 818.55 (M+H)⁺

### Example 69

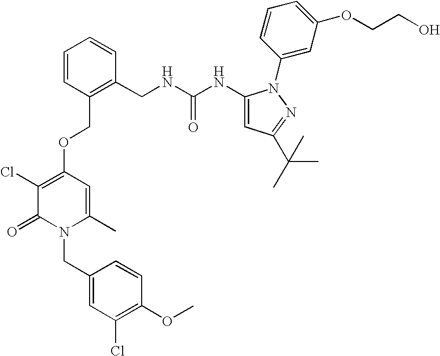

### 1-(2-((1-(3-chloro-4-methoxybenzyl)-3-chloro-1,2-dihydro-6-methyl-2-oxopyridin-4-yloxy)methyl)benzyl)-3-(1-(3-(2-hydroxyethoxy)-phenyl)-3-tert-butyl-1H-pyrazol-5-yl)urea

4-Toluenesulfonic acid (0.065 g, 0.341 mmol) was added to a stirred solution of 1-(2-((1-(3-chloro-4-methoxybenzyl)-3-chloro-1,2-dihydro-6-methyl-2-oxopyridin-4-yloxy)methyl)benzyl)-3-(1-(3-(2-(tetrahydro-2H-pyran-2-yloxy)ethoxy)phenyl)-3-tert-butyl-1H-pyrazol-5-yl)urea (0.557 g, 0.68 mmol) in MeOH (10 mL) and heated at 60° C. for 40 min. The reaction was evaporated and partitioned between EtOAc and saturated sodium bicarbonate. The EtOAc layer was washed with water, dried over MgSO₄ and the solvent was removed to give crude product. The crude was purified by reversed phase LC. The desired fractions were combined, and 1 mL of 5% NaHCO₃ was added, and extracted with CH₂Cl₂. The organic layer was dried over Na₂SO₄ to afford the title compound as a white solid. ¹H NMR (300 MHz, DMSO-d₆) δ 1.22 (s, 9H), 2.29 (s, 3H), 3.70 (m, 2H), 3.81 (s, 3H), 3.99 (m, 2H), 4.35 (m, 2H), 4.87 (m, 1H), 5.21 (s, 2H), 5.32 (s, 2H), 6.24 (s, 1H), 6.53 (s, 1H), 7.01-7.44 (m, 12H), 8.27 (s, 1H); MS (ES+) for C₃₈H₄₁Cl₂N₅O₆ m/z 734.49 (M+H)⁺.

### Intermediate 39i

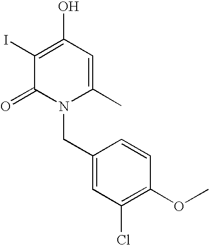

### 1-(3-chloro-4-methoxybenzyl)-4-hydroxy-3-iodo-6-methylpyridin-2(1H)-one

1-(3-Chloro-4-methoxybenzyl)-4-hydroxy-6-methylpyridin-2(1B)-one (8.0 g, 28.0 mmol) was slurried in acetonitrile (300 mL). The mixture was cooled to 0° C. in an ice-water bath. N-iodosuccinimide (6.43 g, 28.0 mmol) was added. The reaction stirred at 0° C. for two hours. The solid was filtered and washed with acetonitrile to give final product 1-(3-chloro-4-methoxybenzyl)-4-hydroxy-3-iodo-6-methylpyridin-2(1H)-one 9.92 g, (87% yield). ¹H NMR (300 MHz, DMSO-d₆) δ 2.20 (s, 3H), 3.80 (s, 3H), 5.17 (s, 2H), 5.92 (s, 1H), 7.02-7.19 (m, 3H), 11.39 (s, 1H); MS (ES+) for C14H13ClINO3 m/z 405.96 (M+H)⁺.

### Intermediate 40i

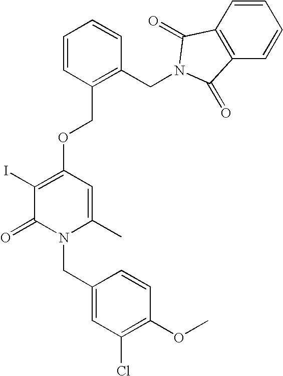

### 2-(2-((1-(3-chloro-4-methoxybenzyl)-1,2-dihydro-3-iodo-6-methyl-2-oxopyridin-4-yloxy)methyl)benzyl)isoindoline-1,3-dione

Potassium carbonate (1.88 g, 13.6 mmol) and 2-[2-(chloromethyl)benzyl]-1H-isoindole-1,3(2H)-dione (3.84 g, 13.6 mmol) were added to a room temperature solution of 1-(3-chloro-4-methoxybenzyl)-4-hydroxy-3-iodo-6-methylpyridin-2(1H)-one (5 g, 12.3 mmol) in DMF (100 mL), then heated at 55° C. under N₂ for 4 h. The reaction was concentrated to approximately ⅓ of the original volume and diluted to 400 mL total volume with water. The solid was filtered, rinsed with additional chilled water and air-dried overnight. This material was triturated with diethyl ether, filtered and air-dried to give 3.5 g (43% yield) of 2-(2-((1-(3-chloro-4-methoxybenzyl)-1,2-dihydro-3-iodo-6-methyl-2-oxopyridin-4-yloxy)methyl)benzyl)isoindoline-1,3-dione as a tan solid. ¹H NMR (300 MHz, DMSO-d₆) δ 2.35 (s, 3H), 3.81 (s, 3H), 4.91 (s, 2H), 5.25 (s, 2H), 5.44 (s, 2H), 6.59 (d, 1H), 7.09 (m, 2H), 7.33 (m, 4H), 7.56 (m, 1H), 7.84 (m, 4H); MS (ES+) for C30H24ClIN2O5 m/z 655.04 (M+H)⁺.

### Intermediate 41i

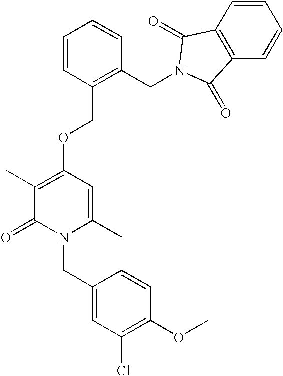

### 2-(2-((1-(3-chloro-4-methoxybenzyl)-1,2-dihydro-3,6-dimethyl-2-oxopyridin-4-yloxy)methyl)benzyl)isoindoline-1,3-dione

2-(2-((1-(3-chloro-4-methoxybenzyl)-1,2-dihydro-3-iodo-6-methyl-2-oxopyridin-4-yloxy)methyl)benzyl)isoindoline-1,3-dione (3.5 g, 5.34 mmol) dissolved in DMF (50 mL). Tetramethyltin (1.54 mL, 2.007 g, 11.2 mmol), lithium chloride (0.792 g, 18.7 mmol) and [1,1′-Bis(diphenylphosphino)ferrocene]-dichloropalladium(II) complex with CH₂Cl₂ (0.436 g, 0.534 mmol) were added. The reaction was stirred overnight at 70° C. It was cooled to room temperature, and ethyl acetate (100 mL) was added. The reaction was extracted with H₂O (50 mL) and brine (50 mL). The organic phase was dried over MgSO₄, filtered, and evaporated. The compound was purified by flash column chromatography to 1.6 g (43% yield) of 2-(2-((1-(3-chloro-4-methoxybenzyl)-1,2-dihydro-3,6-dimethyl-2-oxopyridin-4-yloxy)methyl)benzyl)isoindoline-1,3-dione as a tan solid. ¹H NMR (300 MHz, DMSO-d₆) δ 2.28 (s, 3H), 2.35 (s, 3H), 3.81 (s, 3H), 4.91 (s, 2H), 5.25 (s, 2H), 5.44 (s, 2H), 6.49 (s, 1H), 7.09-7.32 (m, 6H), 7.56 (m, 1H), 7.84 (m, 4H); MS (ES+) for C31H27ClN2O5 m/z 543.16 (M+H)⁺.

### Intermediate 42i

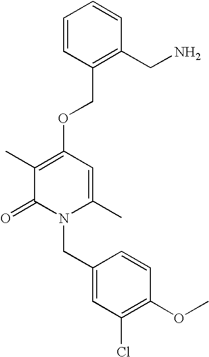

### 4-(2-(aminomethyl)benzyloxy)-1-(3-chloro-4-methoxybenzyl)-3,6-dimethylpyridin-2(1H)-one

Hydrazine hydrate (5.3 mL, 109 mmol) was added to a suspension of compound 2-(2-((1-(3-chloro-4-methoxybenzyl)-1,2-dihydro-3,6-dimethyl-2-oxopyridin-4-yloxy)methyl)benzyl)isoindoline-1,3-dione (1.64 g, 3.02 mmol) in MeOH (100 mL) and stirred at room temperature overnight. The reaction was evaporated and partitioned between EtOAc (250 mL) and NaOH (2.5 N, 125 mL). The EtOAc layer was washed with water, separated, dried over MgSO₄ and evaporated to give 1.05 g (84% yield) of 4-(2-(aminomethyl)benzyloxy)-1-(3-chloro-4-methoxybenzyl)-3,6-dimethylpyridin-2(1H)-one as an off-white solid. ¹H NMR (300 MHz, DMSO-d₆) δ 2.28 (s, 3H), 2.32 (s, 3H), 3.80 (s, 2H), 3.81 (s, 3H), 5.21 (s, 2H), 5.35 (s, 2H), 5.74 (s, 2H), 6.59 (s, 1H), 7.08-7.47 (m, 7H); MS (ES+) for C₂₃H₂₅ClN₂O₃ m/z 413.22 (M+H)⁺.

### General Procedure for Urea Formation:

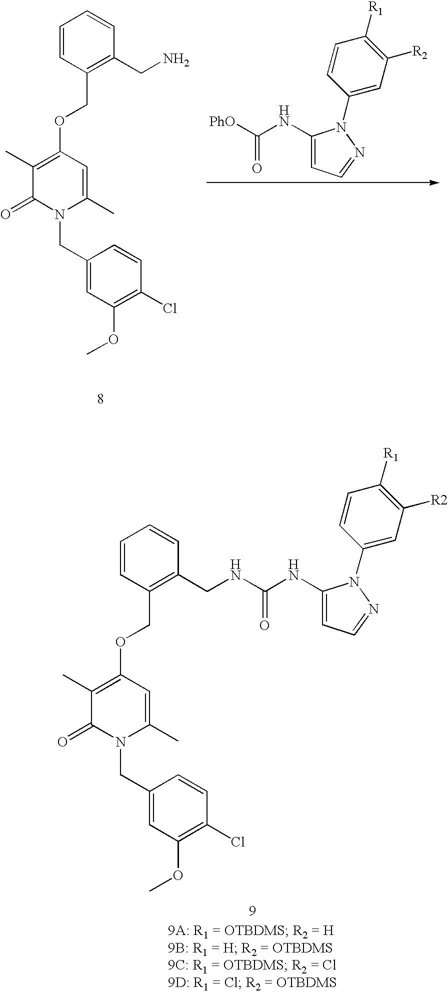

### General Procedure (for Preparation of 9 A-D):

Triethylamine (0.5 mL, 3.59 mmol) and the appropriate carbamate (0.635 mmol) in THF (5 mL) was added to 4-(2-(aminomethyl)benzyloxy)-1-(3-chloro-4-methoxybenzyl)-3,6-dimethylpyridin-2(1H)-one (0.25 g, 0.577 mmol) in THF (5 mL). After bringing the reaction up to 60° C. the reaction was stirred at room temperature overnight. The reaction was partitioned between EtOAc and 2.5 N NaOH, the EtOAc layer was separated, dried over MgSO₄ and the solvent was removed to give the appropriate urea, which was carried on without further purification.

### Example 70

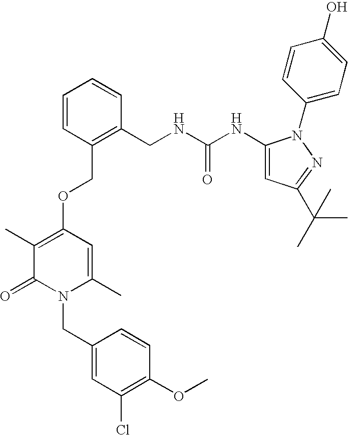

### 1-(2-((1-(3-chloro-4-methoxybenzyl)-1,2-dihydro-3,6-dimethyl-2-oxopyridin-4-yloxy)methyl)benzyl)-3-(3-tert-butyl-1-(4-hydroxyphenyl)-1H-pyrazol-5-yl)urea

Potassium fluoride (0.18 g, 3.11 mmol) was added to crude 9A in MeOH (15 mL) and stirred at room temperature for 1.5 h. The solvent was evaporated, the residue was washed with 0.5N HCl followed by water. The reaction mixture was diluted with ethyl acetate (50 mL) and transferred to a separatory funnel. It was extracted with H₂O (25 mL) and brine (25 mL). The organic phase was dried over MgSO₄, filtered, and evaporated. The compound was purified by flash column chromatography. A white solid was isolated. ¹H NMR (300 MHz, DMSO-d₆) δ 1.20 (s, 9H), 1.88 (s, 3H), 2.24 (s, 3H), 3.80 (s, 3H), 4.31 (d, 2H,), 5.17 (m, 2H), 5.21 (m, 2H), 6.19 (s, 1H), 6.35 (s, 1H), 6.83 (m, 2H), 7.07-7.42 (m, 10H), 8.07 (s, 1H), 9.70 (s, 1H); MS (ES+) for C₃₇H₄₀ClN₅O₅ m/z 670.27 (M+H)⁺.

### Example 71

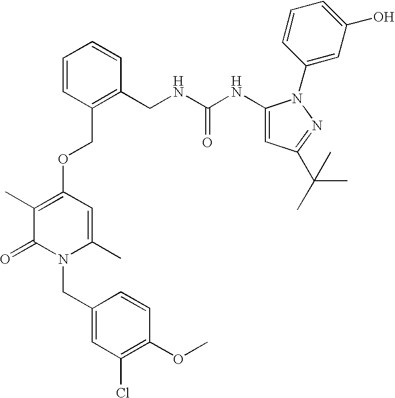

### 1-(2-((1-(3-chloro-4-methoxybenzyl)-1,2-dihydro-3,6-dimethyl-2-oxopyridin-4-yloxy)methyl)benzyl)-3-(3-tert-butyl-1-(3-hydroxyphenyl)-1H-pyrazol-5-yl)urea

1-(2-((1-(3-chloro-4-methoxybenzyl)-1,2-dihydro-3,6-dimethyl-2-oxopyridin-4-yloxy)methyl)benzyl)-3-(3-tert-butyl-1-(3-hydroxyphenyl)-1H-pyrazol-5-yl)urea was prepared in a similar fashion to 1-(2-((1-(3-chloro-4-methoxybenzyl)-1,2-dihydro-3,6-dimethyl-2-oxopyridin-4-yloxy)methyl)benzyl)-3-(3-tert-butyl-1-(4-hydroxyphenyl)-1H-pyrazol-5-yl)urea. ¹H NMR (300 MHz, DMSO-d₆) δ 1.22 (s, 9H), 1.89 (s, 3H), 2.24 (s, 3H), 3.80 (s, 3H), 4.31 (d, 2H), 5.17 (m, 2H), 5.22 (m, 2H), 6.23 (s, 1H), 6.35 (s, 1H), 6.76 (m, 1H), 7.07 (m, 2H), 7.20 (m, 3H), 7.23 (m, 5H), 7.42 (m, 1H), 8.23 (s, 1H), 9.73 (s, 1H); MS (ES+) for C₃₇H₁₀ClN₅O₅ m/z 670.27 (M+H)⁺.

### Example 72

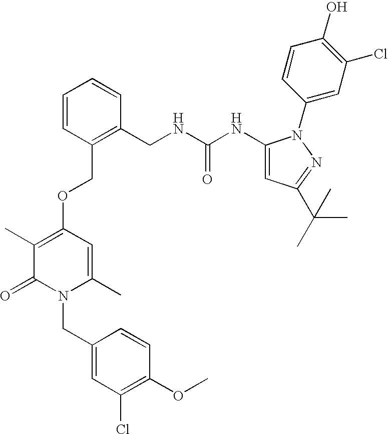

### 1-(2-((1-(3-chloro-4-methoxybenzyl)-1,2-dihydro-3,6-dimethyl-2-oxopyridin-4-yloxy)methyl)benzyl)-3-(3-tert-butyl-1-(3-chloro-4-hydroxyphenyl)-1H-pyrazol-5-yl)urea

1-(2-((1-(3-chloro-4-methoxybenzyl)-1,2-dihydro-3,6-dimethyl-2-oxopyridin-4-yloxy)methyl)benzyl)-3-(3-tert-butyl-1-(3-chloro-4-hydroxyphenyl)-1H-pyrazol-5-yl)urea was prepared in a similar fashion to 1-(2-((1-(3-chloro-4-methoxybenzyl)-1,2-dihydro-3,6-dimethyl-2-oxopyridin-4-yloxy)methyl)benzyl)-3-(3-tert-butyl-1-(4-hydroxyphenyl)-1H-pyrazol-5-yl)urea. ¹H NMR (300 MHz, DMSO-d₆) δ 1.21 (s, 9H), 1.89 (s, 3H), 2.24 (s, 3H), 3.80 (s, 3H), 4.31 (d, 2H), 5.17 (m, 2H), 5.22 (m, 2H), 6.19 (s, 1H), 6.35 (s, 1H), 7.01 (m, 1H), 7.07 (m, 3H), 7.23 (m, 5H), 7.42 (m, 2H), 8.16 (s, 1H), MS (ES+) for C₃₇H₃₉Cl₂N₅O₅ m/z 704.50 (M+H)⁺.

### Example 73

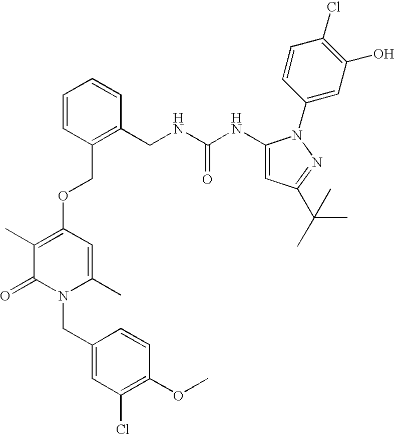

### 1-(2-((1-(3-chloro-4-methoxybenzyl)-1,2-dihydro-3,6-dimethyl-2-oxopyridin-4-yloxy)methyl)benzyl)-3-(3-tert-butyl-1-(4-chloro-3-hydroxyphenyl)-1H-pyrazol-5-yl)urea

1-(2-((1-(3-chloro-4-methoxybenzyl)-1,2-dihydro-3,6-dimethyl-2-oxopyridin-4-yloxy)methyl)benzyl)-3-(3-tert-butyl-1-(4-chloro-3-hydroxyphenyl)-1H-pyrazol-5-yl)urea was prepared in a similar fashion to 1-(2-((1-(3-chloro-4-methoxybenzyl)-1,2-dihydro-3,6-dimethyl-2-oxopyridin-4-yloxy)methyl)benzyl)-3-(3-tert-butyl-1-(4-hydroxyphenyl)-1H-pyrazol-5-yl)urea. ¹H NMR (300 MHz, DMSO-d₆) δ 1.22 (s, 9H), 1.88 (s, 3H), 2.24 (s, 3H), 3.80 (s, 3H), 4.31 (d, 2H), 5.17 (m, 2H), 5.22 (m, 2H), 6.23 (s, 1H), 6.35 (s, 1H), 7.01-7.42 (m, 1H), 8.28 (s, 1H), MS (ES+) for C₃₇H₃₉Cl₂N₅O₅ m/z 704.50 (M+H)⁺.

### Intermediate 43i

### 4-Chloro-3-methoxybenzylamine

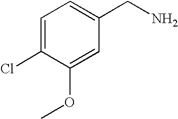

Sulfuryl chloride (13.5 mL, 166.5 mmol) was added drop-wise to a cooled, vigorously stirred solution of 3-methoxybenzylamine (20.76 g, 151 mmole) in glacial acetic acid (300 mL) over 15 minutes, maintaining the reaction temperature <24° C. during the addition. The reaction was warmed to room temperature, diluted with diethyl ether (600 mL) and cooled to −15° C. The resulting solid was filtered, rinsed with additional diethyl ether and air dried to give 19.3 g of white solid. The solid was recrystallized from MeOH (75 mL) and diethyl ether (75 mL) to give 8.25 g (31% yield) of product as the HCl salt. The material was partitioned between EtOAc and saturated sodium bicarbonate, the EtOAc layer was separated, dried over MgSO₄, filtered and evaporated. A 3.67 g portion of the product was purified on silica gel in EtOAc. The product was eluted with 10% MeOH/EtOAc to give 3.1 g (84% yield) of 1-(3-Chloro-4-methoxybenzyl)-4-hydroxy-6-methylpyridin-2(1H)-one as a clear oil.

¹H NMR (300 MHz, CDCl₃) δ 1.83 (s, 2H), 3.79 (s, 3H), 3.89 (s, 2H), 6.73 (m, 1H), 6.94 (m, 1H), 7.24 (m, 1H); MS (ES+) for C₈H₁₀ClNO m/z 172.06 (M+H)⁺.

### REFERENCES

Tetrahedron Letters 42 (2001) 3247-3249.
Synthesis 3, (2003) 403-407.

### Intermediate 44i

### 1-(4-Chloro-3-methoxybenzyl)-4-hydroxy-6-methylpyridin-2(1H)-one

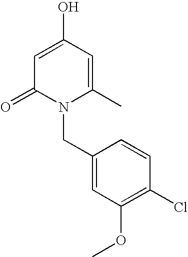

4-Hydroxy-6-methyl-2-pyrone (6.83 g, 54.2 mmol) was dissolved in water (220 mL) at 100° C. 4-Chloro-3-methoxybenzylamine (3.1 g, 18 mmol) was added drop-wise to the above solution over 5 minutes while at 100° C. The reaction was refluxed under N₂ for 5 h. then filtered while still hot and rinsed with additional hot water. The material was air-dried to give 3.8 g (75% yield) of 3-chloro-1-(3-chloro-4-methoxybenzyl)-4-hydroxy-6-methylpyridin-2(1B)-one as a sand-colored solid. ¹H NMR (300 MHz, DMSO-d₆) δ 2.13 (s, 3H), 3.64 (s, 3H), 5.11 (s, 2H), 5.58 (m, 1H), 5.86 (m, 1H), 6.04 (m, 1H), 6.88 (m, 1H), 7.40 (m, 1H), 10.58, (s, 1H); MS (ES+) for C₁₄H₁₄ClNO₃ m/z 280.2 (M+H)⁺.

### Intermediate 45i

### 3-chloro-1-(4-chloro-3-methoxybenzyl)-4-hydroxy-6-methylpyridin-2(1H)-one

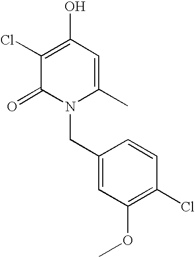

Solid N-chlorosuccinimide (2.06 g, 15.4 mmol) was added to a solution of 1-(4-Chloro-3-methoxybenzyl)-4-hydroxy-6-methylpyridin-2(1H)-one (3.6 g, 12.8 mmol) in a mixture of 1,2-dichloroethane (125 mL) and 2-propanol (90 mL) at 55° C. A second portion of N-chlorosuccinimide (0.15 g, 1.12 mmol) was added after 1 h. After ½ h, the reaction was evaporated to give 6.39 g yellow solid. This material was triturate from methylene chloride (75 mL total volume) and filtered to give 3.32 g (82% yield) of 2-[2-({[3-chloro-1-(3-chloro-4-methoxybenzyl)-6-methyl-2-oxo-1,2-dihydropyridin-4-yl]oxy}methyl)benzyl]-1H-isoindole-1,3(2H)-dione as an off-white solid. ¹H NMR (300 MHz, DMSO-d₆) δ 2.17 (s, 3H), 3.64 (s, 3H), 5.18 (s, 2H), 6.01 (m, 1H), 6.07 (m, 1H), 6.90 (m, 1H), 7.42 (m, 1H), 11.38 (s, 1H); MS (ES+) for C₁₄H₁₃Cl₂NO₃ m/z 314.18 (M+H)⁺.

### Intermediate 46i

### 2-[2-({[3-chloro-1-(4-chloro-3-methoxybenzyl)-6-methyl-2-oxo-1,2-dihydropyridin-4-yl]oxy}methyl)benzyl]-1H-isoindole-1,3(2H)-dione

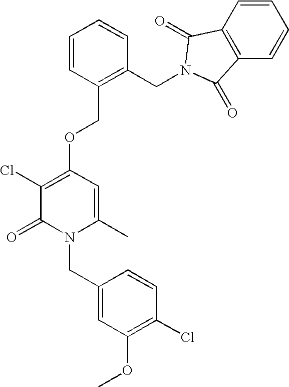

Potassium carbonate (1.45 g, 10.5 mmol) and 2-[2-(chloromethyl)benzyl]-1H-isoindole-1,3(2H)-dione (3 g, 10.5 mmol) were added to a room temperature solution of compound 3-chloro-1-(4-chloro-3-methoxybenzyl)-4-hydroxy-6-methylpyridin-2(1H)-one (3 g, 9.55 mmol) in DMF (60 mL), then heated at 55° C. under N₂ for 4 h. The reaction was concentrated to approximately ⅓ of the original volume and diluted to 400 mL total volume with water. The solid was filtered, rinsed with additional chilled water and air-dried overnight. This material was triturated with diethyl ether, filtered and air-dried to give 4.4 g (81% yield) of 4-{[2-(aminomethyl)benzyl]oxy}-3-chloro-1-(3-chloro-4-methoxybenzyl)-6-methylpyridin-2(1H)-one as a tan solid. ¹H NMR (300 MHz, DMSO-d₆) δ 2.31 (s, 3H), 3.65 (s, 3H), 4.91 (s, 2H), 5.24 (s, 2H), 5.48 (s, 2H), 6.04 (d, 1H), 6.68 (s, 1H), 6.90 (m, 1H), 7.19 (m, 1H), 7.34 (m, 2H), 7.45 (m, 1H), 7.58 (m, 1H), 7.85 (m, 4H); MS (ES+) for C₃₀H₂₄Cl₂N₂O₅ m/z 563.27 (M+H)⁺.

### Intermediate 47i

### 4-{[2-(aminomethyl)benzyl]oxy}-3-chloro-1-(4-chloro-3-methoxybenzyl)-6-methylpyridin-2(1H)-one

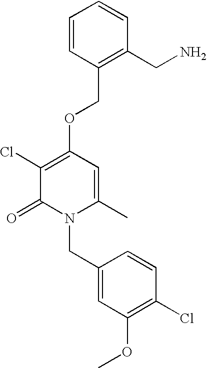

Hydrazine hydrate (10.88 mL, 224 mmol) was added to a suspension of compound 2-[2-({[3-chloro-1-(4-chloro-3-methoxybenzyl)-6-methyl-2-oxo-1,2-dihydropyridin-4-yl]oxy}methyl)benzyl]-1H-isoindole-1,3(2H)-dione (4.2 g, 7.45 mmol) in MeOH (260 mL) and stirred at room temperature overnight. The reaction was evaporated and partitioned between EtOAc (250 mL) and NaOH (2.5 N, 125 mL). The EtOAc layer was washed with water, separated, dried over MgSO₄ and evaporated to give 2.49 g (77% yield) of 4-(2-(Aminomethyl)benzyloxy)-1-(2-methoxybenzyl)-3,6-dimethylpyridin-2(1H)-one as an off-white solid. ¹H NMR (300 MHz, DMSO-d₆) δ 1.78 (br s, 2H), 2.28 (s, 3H), 3.64 (s, 3H), 3.81 (s, 2H), 5.24 (s, 2H), 5.39 (s, 2H), 6.01 (d, 1H), 6.69 (s, 1H), 6.91 (m, 1H), 7.26-7.35 (m, 2H), 7.45 (m, 3H); MS (ES+) for C₂₂H₂₂Cl₂N₂O₃ m/z 433.22 (M+H)⁺.

### General Procedure for Urea Formation:

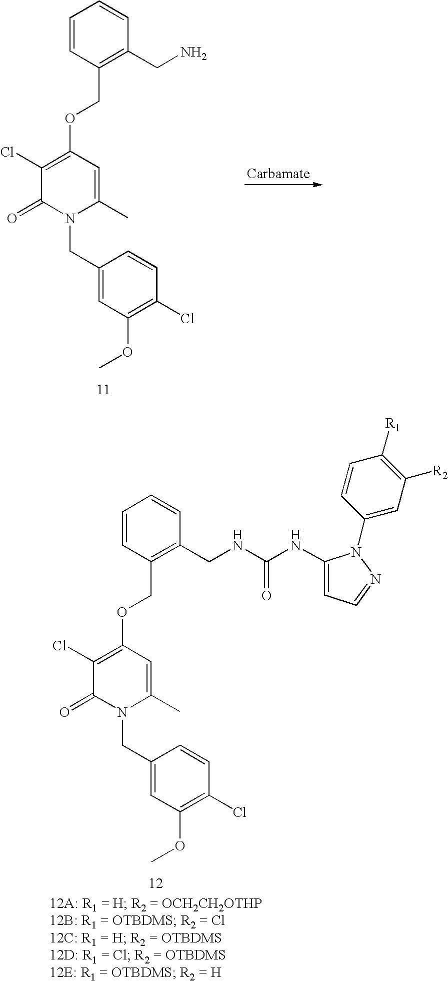

### General Procedure E

Triethylamine (0.5 mL, 3.59 mmol) and the appropriate carbamate (0.635 mmol) in THF (5 mL) was added to compound 4-{[2-(aminomethyl)benzyl]oxy}-3-chloro-1-(4-chloro-3-methoxybenzyl)-6-methylpyridin-2(1H)-one (0.25 g, 0.577 mmol) in THF (5 mL). After bringing the reaction up to 60° C. the reaction was stirred at room temperature overnight. The reaction was partitioned between EtOAc and 2.5 N NaOH, the EtOAc layer was separated, dried over MgSO₄ and the solvent was removed to give the appropriate urea, which was carried on without further purification.

### Example 74

### 1-(3-tert-butyl-1-{3-[2-(tetrahydro-2H-pyran-2-yloxy)ethoxy]phenyl}-1H-pyrazol-5-yl)-3-[2-({[3-chloro-1-(4-chloro-3-methoxybenzyl)-6-methyl-2-oxo-1,2-dihydropyridin-4-yl]oxy}methyl)benzyl]urea

Compound 12A was prepared according to the method of General Procedure E, utilizing an appropriately substituted carbamate. Yield 0.514 g (94% yield) white solid; C18-HPLC t_{R}=10.83 min (87% pure); carried forward without further purification.

### Example 75

### 1-[3-tert-butyl-1-(4-{[tert-butyl(dimethyl)silyl]oxy}-3-chlorophenyl)-1H-pyrazol-5-yl]-3-[2-({[3-chloro-1-(4-chloro-3-methoxybenzyl)-6-methyl-2-oxo-1,2-dihydropyridin-4-yl]oxy}methyl)benzyl]urea

Compound 12B was prepared according to the method of General Procedure E, utilizing the appropriate carbamate. Yield 0.585 g (98% yield) oil; C18-HPLC t_{R}=12.59 min (81% pure); carried forward without further purification.

### Example 76

### 1-[3-tert-butyl-1-(3-{[tert-butyl(dimethyl)silyl]oxy}phenyl)-1H-pyrazol-5-yl]-3-[(2-{[3-chloro-1-(4-chloro-3-methoxybenzyl)-6-methyl-2-oxo-1,2-dihydropyridin-4-yl]oxy}methyl)benzyl]urea

Compound 12C was prepared according to the method of General Procedure E, utilizing the appropriate carbamate. Yield 0.589 g (98% yield) foam; C18-HPLC t_{R}=12.15 min (80% pure); carried forward without further purification.

### General Procedure F

Triethylamine (0.2 mL, 1.43 mmol) and the appropriate carbamate (0.254 mmol) in THF (2 mL) were added to compound 4-{[2-(aminomethyl)benzyl]oxy}-3-chloro-1-(4-chloro-3-methoxybenzyl)-6-methylpyridin-2(1H)-one (0.1 g, 0.23 mmol) in THF (2 mL). The reaction mixture was stirred at 60° C. for 2 hours. The liquid part was removed to give the appropriate urea, which was carried on without further purification.

### Example 77

### 1-[3-tert-butyl-1-(4-{[tert-butyl(dimethyl)silyl]oxy}phenyl)-1H-pyrazol-5-yl]-3-[2-({[3-chloro-1-(4-chloro-3-methoxybenzyl)-6-methyl-2-oxo-1,2-dihydropyridin-4-yl]oxy}methyl)benzyl]urea

Compound 12D was prepared according to the method of General Procedure F, utilizing the appropriate carbamate. Yield 0.23 g (97% yield) foam; C18-HPLC t_{R}=13.06 min (90% pure); carried forward without further purification.

### Example 78

### 1-[3-tert-butyl-1-(3-{[tert-butyl(dimethyl)silyl]oxy}-4-chlorophenyl)-1H-pyrazol-5-yl]-3-[2-({[3-chloro-1-(4-chloro-3-methoxybenzyl)-6-methyl-2-oxo-1,2-dihydropyridin-4-yl]oxy}methyl)benzyl]urea

Compound 12E was prepared according to the method of General Procedure F, utilizing the appropriate carbamate. Yield 0.23 g (96% yield) oil; C18-HPLC t_{R}=12.86 min (89% pure); carried forward without further purification.

### Deprotection of Compounds 12A-E:

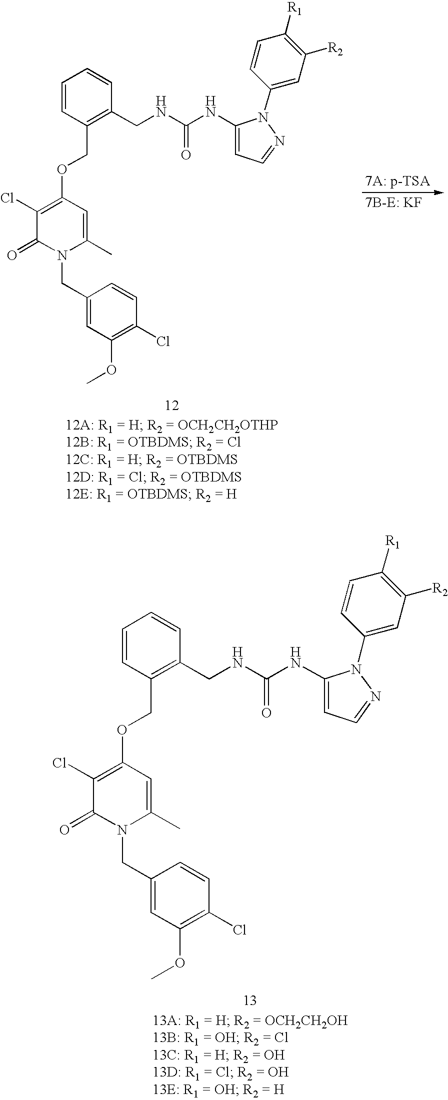

### Example 79

### 1-{3-tert-butyl-1-[3-(2-hydroxyethoxy)phenyl]-1H-pyrazol-5-yl}-3-[2-({[3-chloro-1-(4-chloro-3-methoxybenzyl)-6-methyl-2-oxo-1,2-dihydropyridin-4-yl]oxy}methyl)-benzyl]urea

4-Toluenesulfonic acid (0.058 g, 0.305 mmol) was added to a stirred solution of 12A (0.5 g, 0.61 mmol) in MeOH (10 mL) and heated at 60° C. for 40 min. The reaction was evaporated and partitioned between EtOAc and saturated sodium bicarbonate. The EtOAc layer was washed with water, dried over MgSO₄ and the solvent was removed to give 0.483 g of product. The product was triturated from EtOAc to give 0.323 g (72% yield) of compound 13A as a white solid. ¹H NMR (300 MHz, DMSO-d₆) δ 1.23 (s, 9H), 2.25 (s, 3H), 3.63 (s, 3H), 3.70 (m, 2H), 3.99 (m, 2H), 4.35 (m, 2H), 4.87 (m, 1H), 5.24 (s, 2H), 5.37 (s, 2H), 6.01 (d, 1H), 6.25 (s, 1H), 6.63 (s, 1H), 6.92 (m, 2H), 7.03 (m, 3H), 7.32 (m, 4H), 7.43 (m, 2H), 8.27 (s, 1H); MS (ES+) for C₃₈H₄₁Cl₂N₅O₆ m/z 734.49 (M+H)⁺.

### Example 80

### 1-[3-tert-butyl-1-(3-chloro-4-hydroxyphenyl)-1H-pyrazol-5-yl]-3-[2-({[3-chloro-1-(4-chloro-3-methoxybenzyl)-6-methyl-2-oxo-1,2-dihydropyridin-4-yl]oxy}methyl)-benzyl]urea

Potassium fluoride (0.104 g, 1.78 mmol) was added to 12B (0.5 g, 0.6 mmol) in MeOH (10 mL) and stirred at room temperature for 45 min. The reaction was evaporated and partitioned between EtOAc and 1 N HCl. The aqueous layer was filtered, rinsed with 5% NaHCO₃, rinsed with pure water and air-dried to give 0.224 g (51% yield) of compound 1-(2-((1-(2-(methylthio)benzyl)-3-chloro-6-methyl-2-oxo-1,2-dihydropyridin-4-yloxy)methyl)benzyl)-3-(3-tert-butyl-1-(3-(tert-butyldimethylsilyloxy)-4-chlorophenyl)-1H-pyrazol-5-yl)urea as a white solid. ¹H NMR (300 MHz, DMSO-d₆) δ 1.21 (s, 9H), 2.26 (s, 3H), 3.63 (s, 3H), 4.34 (m, 2H), 5.23 (s, 2H), 5.37 (s, 2H), 6.01 (d, 1H), 6.20 (s, 1H), 6.64 (s, 1H), 6.93-7.03 (m, 3H), 7.30-7.45 (m, 8H), 8.18 (s, 1H); MS (ES+) for C₃₆H₃₆Cl₃N₅O₅ m/z 726.44 (M+H)⁺.

### Example 81

### 1-[3-tert-butyl-1-(3-hydroxyphenyl)-1H-pyrazol-5-yl]-3-[2-({[3-chloro-1-(4-chloro-3-methoxybenzyl)-6-methyl-2-oxo-1,2-dihydropyridin-4-yl]oxy}methyl)benzyl]urea

Potassium fluoride (0.10 g, 1.73 mmol) was added to 12C (0.464 g, 0.57 mmol) in MeOH (10 mL) and stirred at room temperature for 1.5 h. The reaction was evaporated, partitioned between EtOAc and 1 N HCl and the EtOAc layer was washed with 5% NaHCO₃ followed by water and dried over MgSO₄. The solvent was removed to give 0.525 g of product. The product was triturated from EtOAc to give 0.38 g (95% yield) of compound 13C as a white solid. ¹H NMR (300 MHz, DMSO-d₆) δ 1.22 (s, 9H), 2.26 (s, 3H), 3.63 (s, 3H), 4.36 (m, 2H), 5.24 (s, 2H), 5.38 (s, 2H), 6.01 (d, 1H), 6.23 (s, 1H), 6.64 (s, 1H), 6.75 (m, 1H), 6.88 (m, 3H), 7.03 (m, 1H), 7.32 (m, 4H), 7.43 (m, 2H), 8.25 (s, 1H) 9.79 (s, 1H); MS (ES+) for C₃₆H₃₇Cl₂N₅O₅ m/z 690.48 (M+H)⁺.

### Example 82

### 1-(2-((1-(4-chloro-3-methoxybenzyl)-3-chloro-1,2-dihydro-6-methyl-2-oxopyridin-4-yloxy)methyl)benzyl)-3-(3-tert-butyl-O-(4-hydroxyphenyl)-1H-pyrazol-5-yl)urea

Potassium fluoride (0.18 g, 3.11 mmol) was added to crude 12D in MeOH (15 mL) and stirred at room temperature for 1.5 h. The solvent was evaporated, the residue was washed with 0.5N HCl followed by water. A preparative chromatograph (Gilson) with reverse phase was used for purification to give 0.089 g of product as a white solid. ¹H NMR (300 MHz, DMSO-d₆) δ 1.23 (s, 9H), 2.25 (s, 3H), 3.65 (s, 3H), 4.36 (d, 2H, J=5.7 Hz), 5.24 (s, 2H), 5.38 (s, 2H), 6.03 (d, 1H, J=2.9 Hz), 6.21 (s, 1H), 6.63 (s, 1H), 6.83 (m, 2H), 6.92 (m, 2H), 7.20 (m, 2H), 7.30 (m, 3H), 7.48 (m, 2H), 8.15 (s, 1H) 9.86 (s, 1H); MS (ES+) for C₃₆H₃₇Cl₂N₅O₅ m/z 690.48 (M+H)⁺.

### Example 83

### 1-(2-((1-(4-chloro-3-methoxybenzyl)-3-chloro-1,2-dihydro-6-methyl-2-oxopyridin-4-yloxy)methyl)benzyl)-3-(3-tert-butyl-1-(4-chloro-3-hydroxyphenyl)-1H-pyrazol-5-yl)urea

Potassium fluoride (0.18 g, 3.11 mmol) was added to crude 12E in MeOH (15 mL) and stirred at room temperature for 1.5 h. The solvent was evaporated, the residue was washed with 0.5N HCl followed by water. A preparative chromatograph (Gilson) with reverse phase was used for purification to give 0.089 g of product as a white solid. ¹H NMR (300 MHz, DMSO-d₆) δ 1.22 (s, 9H), 2.26 (s, 3H), 3.63 (s, 3H), 4.36 (d, 2H, J=5.6 Hz), 5.24 (s, 2H), 5.37 (s, 2H), 6.03 (d, 1H, J=2.9 Hz), 6.24 (s, 1H), 6.64 (s, 1H), 6.95 (m, 3H), 7.13 (m, 1H), 7.48 (m, 6H), 8.25 (s, 1H), 9.86 (s, 1H); MS (ES+) for C₃₆H₃₆Cl₃N₅O₅ m/z 726.44 (M+H)⁺.

### Example 84

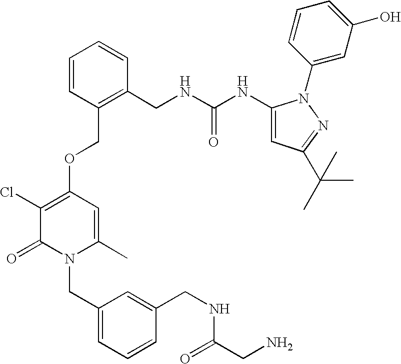

### 1-(2-((1-(3-((2-aminoacetamido)methyl)benzyl)-3-chloro-6-methyl-2-oxo-1,2-dihydropyridin-4-yloxy)methyl)benzyl)-3-(3-tert-butyl-1-(3-hydroxyphenyl)-1H-pyrazol-5-yl)urea

### Step 1: synthesis of the intermediate tert-butyl 3-((4-hydroxy-6-methyl-2-oxopyridin-1(2H)-yl)methyl)benzylcarbamate

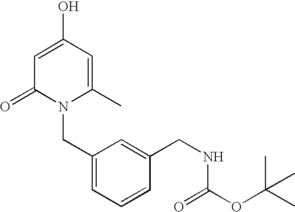

A 5 L RBF equipped with an overhead stirrer, bottom drain, lower mantle, and internal thermometer was charged with 4-hydroxy-6-methyl-2H-pyran-2-one (25.22 g, 200.0 mmol) and 1-(N—BOC-aminomethyl)-3-(aminomethyl)benzene (52.66 g, 200.0 mmol) and then H₂O (800 mL). The reaction internal temperature was brought to 60° C. for 20 minutes to ensure most solids dissolved. At this time the reaction appeared milky white during overhead stirring. The reaction mixture was then stirred at 98° C. for 3.5 hours. At this time the internal temperature was allowed to drop on its own accord to 60° C. and the water was drained from the vessel through the bottom drain device, leaving behind a caramel colored residue that coated the inside of the reaction vessel. Next, 3.2 L of methylene chloride was added to the reaction vessel and upon rigorous stirring the solid residue completely dissolved. This resultant solution was treated with 1.4 L of 1.0 N NaOH solution and the slurry was stirred for 25 minutes and then allowed to stand for 6 hours. Upon standing the water layer separated, was collected to a 4 L chamber, and treated with 110 mL of 12 M HCl until roughly PH-3 or PH-4 (based upon paper strip analysis). Upon this adjustment of PH, an oily solid developed that was collected. The resulting semi-solid was suspended in ethyl acetate/MeOH (1:1 ratio, 1.0 L) and concentrated by nitrogen stream to furnish a tan solid that was further dried under vacuum to constant weight of 33.1 g (48%).

¹H NMR (400 MHz, methanol-d4) δ ppm: 1.42 (s, 9H), 2.23 (s, 3H), 4.17 (s, 2H), 5.30 (s, 2H), 5.79 (d, J=2.5 Hz, 1H), 5.94 (app d, J=2.1 Hz, 1H), 7.04-6.91 (m, 2H), 7.16 (d, J=7.5 Hz, 1H), 7.28 (app t, J=7.5 Hz, 1H). HRMS (m/z) 345.1791. M+H, C₁₉H₂₅N₂O₄ requires 345.1809. LC/MS (C-18 column, gradient elution 5 minute chromatograph, 95:5 to 5:95 water/acetonitrile, retention time 2.34 min).

### Step 2: synthesis of the intermediate tert-butyl 3-((4-hydroxy-3-iodo-6-methyl-2-oxopyridin-1(2H)-yl)methyl)benzylcarbamate

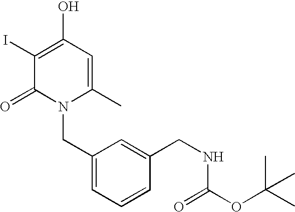

A 2 L round bottom flask was charged with a slurry of the previous intermediate, tert-butyl 3-((4-hydroxy-6-methyl-2-oxopyridin-1(2H)-yl)methyl)benzylcarbamate (33.1 g, 96.1 mmol) and 800 mL of methylene to chloride. To the rapidly stirring mixture was added N-iodosuccinimide (22.5 g, 100 mmol) portionwise over 5 minutes being cautious not to let the internal reaction temperature exceed 25° C. After 3 hours of reaction time, the resulting reddish solution was concentrated to a powder residue using a continuous nitrogen stream. The resulting solid was transferred to a Buchner-filter apparatus, and the dark colored solid was washed with 0° C. acetonitrile (75 mL) that provided a resulting off-white solid (42.5 g, 94%). ¹H NMR (400 MHz, methanol-d4) δ ppm: 1.41 (s, 9H), 2.23 (s, 3H), 4.17 (s, 2H), 5.37 (s, 2H), 5.99 (s, 1H), 6.98 (br s, 2H) 7.15 (d, J=7.9 Hz, 1H), 7.26 (app t, J=7.7 Hz, 1H). HRMS (m/z) 471.0716. M+H, C₁₉H₂₄IN₂O₄ requires 471.0775. LC/MS (C-18 column, gradient elution 5 minute chromatograph, 95:5 to 5:95 water/acetonitrile, retention time 2.47 min).

### Step 3: synthesis of intermediate tert-butyl 3-((3-chloro-4-hydroxy-6-methyl-2-oxopyridin-1(2H)-yl)methyl)benzylcarbamate

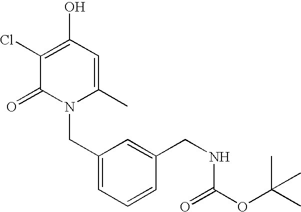

A 500 mL round bottom flask was charged with the previous intermediate, tert-butyl 3-((4-hydroxy-3-iodo-6-methyl-2-oxopyridin-1(2H)-yl)methyl)benzylcarbamate (20.6 g, 43.8 mmol) and 100 mL of DMF. To the resulting solution was added lithium chloride (50.0 g, 1100 mmol). The resulting slurry was heated to 83° C. and stirred for 10 hours. At this time the reaction was allowed to cool to room temperature on its own accord and stand for 12 hours further. Next, the mixture was poured into 1.2 L of rapidly stirring water and an off-white solid precipitated, was collected and allowed to air dry (15.0 g, 90%). ¹H NMR (400 MHz, methanol-d4) δ ppm: 1.41 (s, 9H), 2.25 (s, 3H), 4.18 (s, 2H), 5.36 (s, 2H) 6.05 (s, 1H), 6.99 (br s, 2H) 7.18 (d, J=7.9 Hz, 1H), 7.26 (app t, J=7.7 Hz, 1H). HRMS (m/z) 379.1400. M+H, C₁₉H₂₄ClN₂O₄ requires 379.1419. LC/MS (C-18 column, gradient elution 5 minute chromatograph, 95:5 to 5:95 water/acetonitrile, retention time 2.41 min).

### Step 4: tert-butyl 3-((4-(2-((1,3-dioxoisoindolin-2-yl)methyl)benzyloxy)-3-chloro-6-methyl-2-oxopyridin-1(2H)-yl)methyl)benzylcarbamate

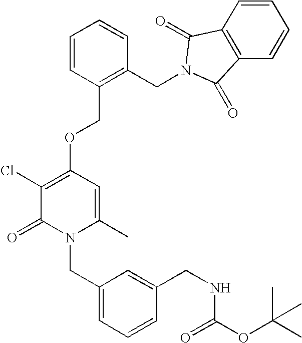

A 1 L round bottom flask was charged with the intermediate from the previous step, tert-butyl 3-((3-chloro-4-hydroxy-6-methyl-2-oxopyridin-1(2H)-yl)methyl)benzylcarbamate (30.0 g, 79.2 mmol) and DMF (350 mL). Next, 2-(2-(chloromethyl)benzyl)isoindoline-1,3-dione (22.6 g, 79.1 mmol) and potassium carbonate (30.0 g, 217 mmol) were added sequentially. The reaction mixture was stirred at room temperature for 12 hours. The reaction is poured directly into rapidly stirring water (1.2 L) and precipitated to provide a white solid that is collected and allowed to air dry (25.1 g, 50%). ¹HNMR (400 MHz, methanol-d4) δ ppm 1.40 (s, 9H), 2.38 (s, 3H), 4.18 (s, 2H), 4.84 (s, 2H), 5.39 (s, 2H), 5.47 (s, 2H), 6.57 (s, 1H), 7.03-6.99 (m, 2H), 7.19 (d, J=7.9 Hz, 1H), 7.47-7.23 (m, 4H), 7.83-7.70 (m, 5H).

HRMS (m/z) 628.2235. M+H, C₃₅H₃₅ClN₃O₆ requires 628.2209. LC/MS (C-18 column, gradient elution 5 minute chromatograph, 95:5 to 5:95 water/acetonitrile, retention time 3.49 min).

### Step 5: synthesis of the intermediate 2-(2-((1-(3-(aminomethyl)benzyl)-3-chloro-6-methyl-2-oxo-1,2-dihydropyridin-4-yloxy)methyl)benzyl)isoindoline-1,3-dione

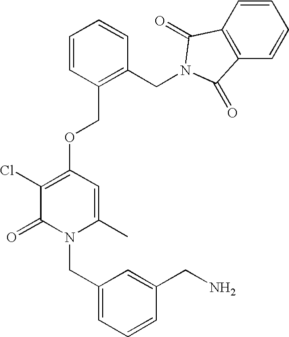

A 500 mL round bottom flask was charged with the intermediate from the previous step, tert-butyl 3-((4-(2-((1,3-dioxoisoindolin-2-yl)methyl)benzyloxy)-3-chloro-6-methyl-2-oxopyridin-1(2H)-yl)methyl)benzylcarbamate (25.0 g, 39.8 mmol) and a commercial solution (Aldrich) of 4 N HCl in 1,4 dioxane (125 mL, 500 mmol). After roughly 1 hour the reaction mixture was quenched by portionwise addition of solid potassium carbonate (125 g, 906 mmol). The resulting reaction mixture was poured directly into rapidly stirring water (2.9 L) and a precipitate was collected as a white solid (19.98 g, 95%). ¹HNMR (400 MHz, methanol-d4) δ ppm: 1.99 (s, 3H), 4.06 (s, 2H), 4.98 (s, 2H), 5.41 (s, 2H), 5.52 (s, 2H), 6.60 (s, 1H), 7.25-7.21 (m, 2H), 7.39-7.32 (m, 3H), 7.49-7.40 (m, 3H), 7.82-7.71 (m, 4H). HRMS (m/z) 528.1694. M+H, C₃₀H₂₇ClN₃O₄ requires 528.1685. LC/MS (C-18 column, gradient elution 5 minute chromatograph, 95:5 to 5:95 water/acetonitrile, retention time 2.49 min).

### Step 6: synthesis of intermediate tert-butyl 2-(3-((4-(2-(aminomethyl)benzyloxy)-3-chloro-6-methyl-2-oxopyridin-1(2H)-yl)methyl)benzylamino)-2-oxoethylcarbamate

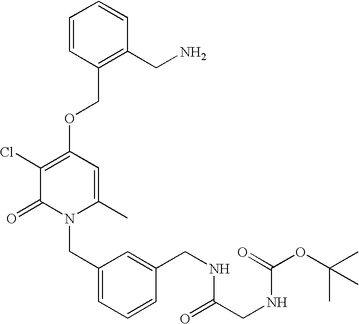

A 500 mL round bottom flask was charged with a solution of commercially available N-(Boc)-Glycine (Aldrich compound number 134538, 8.76 g, 50.0 mmol) in THF (200 mL). Next was added N-methyl morpholine (50.0 mL, 453 mmol) and 2-chloro-4,6-dimethyoxy-1,3,5-triazine (10.5 g, 60.0 mmol). The reaction suspension became a yellow solution and was allowed to stir at room temperature for 1 hour. Next was added the intermediate from the previous step, 2-(2-((1-(3-(aminomethyl)benzyl)-3-chloro-6-methyl-2-oxo-1,2-dihydropyridin-4-yloxy)methyl)benzyl)isoindoline-1,3-dione (5.28 g, 10.0 mmol). After roughly 0.5 hour the reaction mixture was quenched by the addition of 200 mL of MeOH and then treated with hydrazine-monohydrate (2.20 mL, 44.0 mmol). The reaction suspension was heated to 60° C. for 10 minutes, stripped to a residue by nitrogen stream, and the resulting residue was filtered and purified by reverse phase C-18 chromatography using a 15 minute run and water/acetonitrile (95:5 to 5:95) gradient solvent system stabilized with 0.1% TFA. The resulting title compound was filtered through an exchange resin (StratoSphere SPE, PL-HCO3 MP-Resin, product number 3540-C603) to remove any TFA salts and provide the designated intermediate as its parent compound (2.62 g, 47%). ¹HNMR (400 MHz, methanol-d4) δ ppm: 1.42 (s, 9H), 2.38 (d, J=5.0 Hz, 3H), 3.70 (br s, 2H), 4.33 (s, 2H), 5.39 (s, 2H), 5.42 (s, 2H), 6.60 (s, 1H), 7.28-6.96 (m, 4H), 7.62-7.43 (m, 4H). HRMS (m/z) 555.2365. M+H, C₂₉H₃₆ClN₄O₅ requires 555.2369. LC/MS (C-18 column, gradient elution 5 minute chromatograph, 95:5 to 5:95 water/acetonitrile, retention time 2.15 min).

### Step 7: Preparation of the Title Compound

A 100 mL round bottom flask was charged with 3-(5-amino-3-tert-butyl-1H-pyrazol-1-yl)phenol (231 mg, 1.00 mmol) and methylene chloride (10 mL). To this suspension was added saturated aqueous sodium bicarbonate solution (10 mL) and a commercially available toluene solution of phosgene (Fluka, 20% concentration, 1 mL, roughly 1.8 mmol). After 20 minutes the reaction emulsion separated into two layers, and the organic extract was concentrated by nitrogen stream to a residue and then suspended in THF (1.0 mL). To this resulting solution was added a solution of the previous intermediate, tert-butyl 2-(3-((4-(2-(aminomethyl)benzyloxy)-3-chloro-6-methyl-2-oxopyridin-1(2H)-yl)methyl)benzylamino)-2-oxoethylcarbamate (250 mg, 0.450 mmol) in THF (5 mL). After 1 hour, the reaction was concentrated to a residue and suspended in commercially available HCl dioxane solution (Aldrich, 4 N in 1,4 dioxane, 5 mL, 20 mmol). The reaction suspension was stripped to a residue by nitrogen stream, and the resulting residue was filtered and purified by reverse phase C-18 chromatography using a 15 minute run and water/acetonitrile (95:5 to 5:95) gradient solvent system stabilized with 0.1% TFA. The resulting title compound was obtained as its mono-TFA salt (65 mg, 17%). ¹HNMR (400 MHz, methanol-d4) δ ppm: 1.28 (s, 9H), 2.38 (d, J=5.0 Hz, 3H), 3.64 (br s, 2H), 4.38 (s, 2H), 4.42 (s, 2H), 5.31 (s, 2H), 5.39 (s, 2H), 6.28 (d, J=7.2 Hz, 1H), 6.51 (s, 1H), 6.89-6.80 (m, 3H), 7.58-7.04 (m, 9H). HRMS (m/z) 712.2999. M+H, C₃₈H₄₃ClN₇O₅ requires 712.3009. LC/MS (C-18 column, gradient elution 5 minute chromatograph, 95:5 to 5:95 water/acetonitrile, retention time 2.51 min).

### Example 85

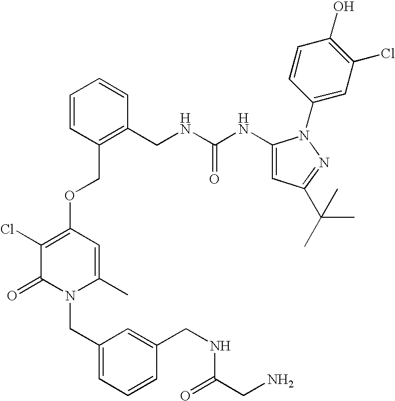

### 1-(2-((1-(3-((2-aminoacetamido)methyl)benzyl)-3-chloro-6-methyl-2-oxo-1,2-dihydropyridin-4-yloxy)methyl)benzyl)-3-(3-tert-butyl-1-(3-chloro-4-hydroxyphenyl)-1H-pyrazol-5-yl)urea

### Step 1: synthesis of the intermediate tert-butyl 2-(3-((4-(2-((3-(3-tert-butyl-1-(3-chloro-4-hydroxyphenyl)-1H-pyrazol-5-yl)ureido)methyl)benzyloxy)-3-chloro-6-methyl-2-oxopyridin-1(2H)-yl)methyl)benzylamino)-2-oxoethylcarbamate

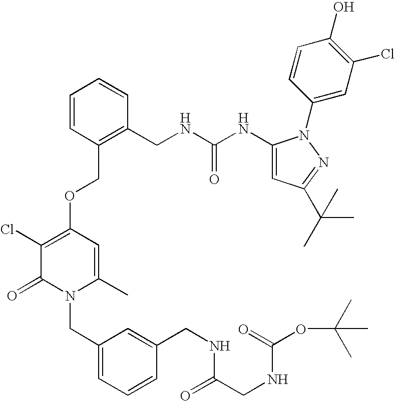

A 100 mL round bottom flask was charged with 4-(5-amino-3-tert-butyl-1H-pyrazol-1-yl)-2-chlorophenol (1.32 g, 5.00 mmol) and methylene chloride (30 mL). To this suspension was added saturated aqueous sodium bicarbonate solution (40 mL) and a commercially available toluene solution of phosgene (Fluka, 20% concentration, 3.0 mL, roughly 5.4 mmol). After 20 minutes the reaction emulsion separated into two layers, and the organic extract was concentrated by nitrogen stream to a residue and then suspended in THF (5.0 mL). To this resulting solution was added a solution of the intermediate tert-butyl 2-(3-((4-(2-(aminomethyl)benzyloxy)-3-chloro-6-methyl-2-oxopyridin-1(2H)-yl)methyl)benzylamino)-2-oxoethylcarbamate (555 mg, 1.00 mmol) in THF (5 mL). After 1 hour, the reaction was concentrated to a residue and was filtered and purified by reverse phase C-18 chromatography using a 15 minute run and water/acetonitrile (95:5 to 5:95) gradient solvent system stabilized with 0.1% TFA. The resulting intermediate compound was obtained as its mono-TFA salt (251 mg, 26%). HRMS (m/z) 846.3137. M+H, C₃₈H₅₀Cl₂N₇O₇ requires 846.3143. LC/MS (C-18 column, gradient elution 5 minute chromatograph, 95:5 to 5:95 water/acetonitrile, retention time 3.21 min).

### Step 2: Preparation of the Title Compound

A 100 mL round bottom flask was charged with the previous intermediate, tert-butyl 2-(3-((4-(2-((3-(3-tert-butyl-1-(3-chloro-4-hydroxyphenyl)-1H-pyrazol-5-yl)ureido)methyl)benzyloxy)-3-chloro-6-methyl-2-oxopyridin-1(2H)-yl)methyl)benzylamino)-2-oxoethylcarbamate (250 mg, 0.260 mmol) and suspended in commercially available HCl dioxane solution (Aldrich, 4 N in 1,4 dioxane, 5 mL, 20 mmol). The reaction suspension was stripped to a residue by nitrogen stream, and the resulting residue was filtered and purified by reverse phase C-18 chromatography using a 15 minute run and water/acetonitrile (95:5 to 5:95) gradient solvent system stabilized with 0.1% TFA. The resulting title compound was obtained as its mono-TFA salt (110 mg, 49%). ¹HNMR (400 MHz, methanol-d4) δ ppm: 1.28 (s, 9H), 2.38 (s, 3H), 3.64 (s, 2H), 4.38 (s, 2H), 4.42 (s, 2H), 5.34 (s, 2H), 5.39 (s, 2H), 6.25 (s, 1H), 6.54 (s, 1H), 6.95 (app d, J=12.5 Hz, 2H), 7.04 (app d, J=8.0 Hz, 1H), 7.08 (s, 1H), 7.38-7.17 (m, 5H), 7.40 (app d, J=3.5 Hz, 1H), 7.46 (app d, J=7.0 Hz, 1H). HRMS (m/z) 746.2620. M+H, C₃₈H₄₂Cl₂N₇O₅ requires 746.2619. LC/MS (C-18 column, gradient elution 5 minute chromatograph, 95:5 to 5:95 water/acetonitrile, retention time 2.63 min).

### Example 86

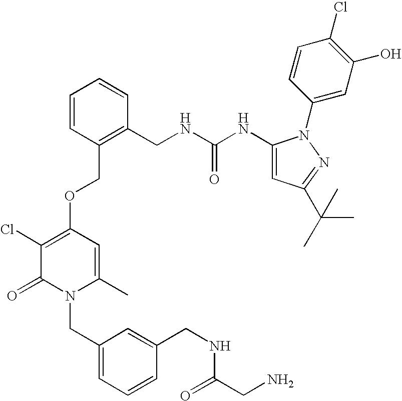

### 1-(2-((1-(3-((2-aminoacetamido)methyl)benzyl)-3-chloro-6-methyl-2-oxo-1,2-dihydropyridin-4-yloxy)methyl)benzyl)-3-(3-tert-butyl-1-(4-chloro-3-hydroxyphenyl)-1H-pyrazol-5-yl)urea

### Step 1: synthesis of the intermediate tert-butyl 2-(3-((4-(2-((3-(3-tert-butyl-1-(4-chloro-3-hydroxyphenyl)-1H-pyrazol-5-yl)ureido)methyl)benzyloxy)-3-chloro-6-methyl-2-oxopyridin-1(2H)-yl)methyl)benzylamino)-2-oxoethylcarbamate

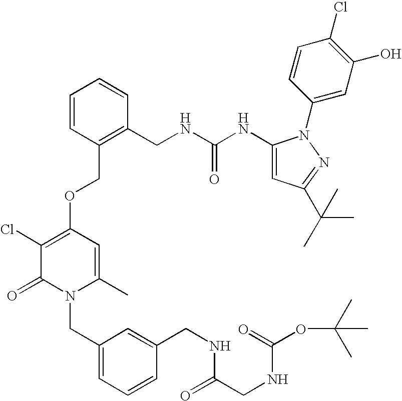

A 100 mL round bottom flask was charged with 5-(5-amino-3-tert-butyl-1H-pyrazol-1-yl)-2-chlorophenol (1.32 g, 5.00 mmol) and methylene chloride (30 mL). To this suspension was added saturated aqueous sodium bicarbonate solution (40 mL) and a commercially available toluene solution of phosgene (Fluka, 20% concentration, 3.0 mL, roughly 5.4 mmol). After 20 minutes the reaction emulsion separated into two layers, and the organic extract was concentrated by nitrogen stream to a residue and then suspended in THF (5.0 mL). To this resulting solution was added a solution of the intermediate tert-butyl 2-(3-((4-(2-(aminomethyl)benzyloxy)-3-chloro-6-methyl-2-oxopyridin-1(2H)-yl)methyl)benzylamino)-2-oxoethylcarbamate (555 mg, 1.00 mmol) in THF (5 mL). After 1 hour, the reaction was concentrated to a residue and was filtered and purified by reverse phase C-18 chromatography using a 15 minute run and water/acetonitrile (95:5 to 5:95) gradient solvent system stabilized with 0.1% TFA. The resulting intermediate compound was obtained as its mono-TFA salt (268 mg, 28%). HRMS (m/z) 846.3153. M+H, C₃₈H₅₀Cl₂N₇O₇ requires 846.3143. LC/MS (C-18 column, gradient elution 5 minute chromatograph, 95:5 to 5:95 water/acetonitrile, retention time 3.26 min).

### Step 2: Preparation of the Title Compound

A 100 mL round bottom flask was charged with the previous intermediate, tert-butyl 2-(3-((4-(2-((3-(3-tert-butyl-1-(4-chloro-3-hydroxyphenyl)-1H-pyrazol-5-yl)ureido)methyl)benzyloxy)-3-chloro-6-methyl-2-oxopyridin-1(2H)-yl)methyl)benzylamino)-2-oxoethylcarbamate (267 mg, 0.278 mmol) and suspended in commercially available HCl dioxane solution (Aldrich, 4 N in 1,4 dioxane, 5 mL, 20 mmol). The reaction suspension was stripped to a residue by nitrogen stream, and the resulting residue was filtered and purified by reverse phase C-18 chromatography using a 15 minute run and water/acetonitrile (95:5 to 5:95) gradient solvent system stabilized with 0.1% TFA. The resulting title compound was obtained as its mono-TFA salt (100 mg, 42%). ¹HNMR (400 MHz, methanol-d4) δ ppm: 1.28 (s, 9H), 2.36 (s, 3H), 3.63 (s, 2H), 4.37 (s, 2H), 4.41 (s, 2H), 5.32 (s, 2H), 5.39 (s, 2H), 6.24 (s, 1H), 6.50 (s, 1H), 6.88 (app dd, J=12.0, 2.1 Hz, 2H), 7.10-7.00 (m, 3H), 7.21 (app d, J=7.8 Hz, 1H), 7.38-7.28 (m, 4H), 7.44 (app d, J=7.0 Hz, 1H). HRMS (m/z) 746.2593. M+H, C₃₈H₄₂Cl₂N₇O₅ requires 746.2619. LC/MS (C-18 column, gradient elution 5 minute chromatograph, 95:5 to 5:95 water/acetonitrile, retention time 2.63 min).

### Example 87

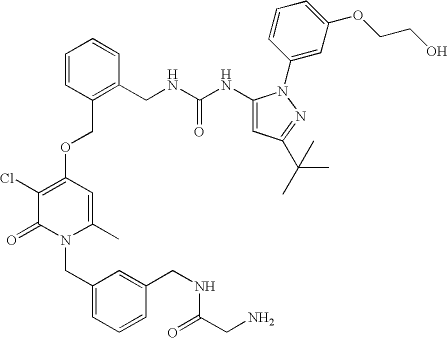

### 1-(2-((1-(3-((2-aminoacetamido)methyl)benzyl)-3-chloro-6-methyl-2-oxo-1,2-dihydropyridin-4-yloxy)methyl)benzyl)-3-(3-tert-butyl-1-(3-(2-hydroxyethoxy)phenyl)-1H-pyrazol-5-yl)urea

### Step 1: Synthesis of the Title Compound

The title compound was prepared in an identical fashion to that of the example 1-(2-((1-(3-((2-aminoacetamido)methyl)benzyl)-3-chloro-6-methyl-2-oxo-1,2-dihydropyridin-4-yloxy)methyl)benzyl)-3-(3-tert-butyl-1-(3-hydroxyphenyl)-1H-pyrazol-5-yl)urea, with a substitution in step 7 of the reagent 3-(5-amino-3-tert-butyl-1H-pyrazol-1-yl)phenol with the reagent 3-tert-butyl-1-(3-(2-(tetrahydro-2H-pyran-2-yloxy)ethoxy)phenyl)-1H-pyrazol-5-amine. The title compound was obtained as its mono-TFA salt (109 mg, 13% yield final step).

¹HNMR (400 MHz, methanol-d4) δ ppm: 1.29 (s, 9H), 2.34 (s, 3H), 3.62 (br s, 2H), 3.81 (t, J=8.2 Hz, 2H), 4.03 (t, J=8.2 Hz, 2H), 4.39 (s, 2H), 4.42 (s, 2H), 5.32 (s, 2H), 5.40 (s, 2H), 6.30 (s, 1H), 6.51 (s, 1H), 7.17-6.94 (m, 3H), 7.20 (d, J=7.2 Hz, 1H), 7.38-7.24 (m, 7H), 7.45 (d, J=7.1 Hz, 1H). HRMS (m/z) 756.3236. M+H, C₄₀H₄₇ClN₇O₆ requires 756.3271. LC/MS (C-18 column, gradient elution 5 minute chromatograph, 95:5 to 5:95 water/acetonitrile, retention time 2.68 min).

### Example 88

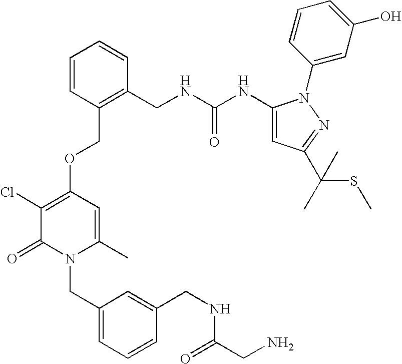

### 1-(2-((1-(3-((2-aminoacetamido)methyl)benzyl)-3-chloro-6-methyl-2-oxo-1,2-dihydropyridin-4-yloxy)methyl)benzyl)-3-(1-(3-hydroxyphenyl)-3-(2-(methylthio)propan-2-yl)-1H-pyrazol-5-yl)urea

### Step 1: Synthesis of the Title Compound

The title compound was prepared in an identical fashion to that of the example 1-(2-((1-(3-((2-aminoacetamido)methyl)benzyl)-3-chloro-6-methyl-2-oxo-1,2-dihydropyridin-4-yloxy)methyl)benzyl)-3-(3-tert-butyl-1-(3-hydroxyphenyl)-1H-pyrazol-5-yl)urea, with a substitution in step 7 of the reagent 3-(5-amino-3-tert-butyl-1H-pyrazol-1-yl)phenol with the reagent 3-(5-amino-3-(2-(methylthio)propan-2-yl)-1H-pyrazol-1-yl)phenol. The title compound was obtained as its mono-TFA salt (114 mg, 14% yield final step). ¹HNMR (400 MHz, methanol-d4) δ ppm: 1.61 (s, 6H), 1.90 (s, 3H), 2.35 (s, 3H), 3.67 (br s, 2H), 4.34 (s, 2H), 4.46 (s, 2H), 5.31 (s, 2H), 5.38 (s, 2H), 6.39 (s, 1H), 6.50 (s, 1H), 6.82 (dd, J=8.5, 7.0 Hz, 1H), 6.89 (br s, 2H), 7.08-7.01 (m, 2H), 7.38-7.18 (m, 6H), 7.47 (d, J=6.8 Hz, 1H). HRMS (m/z) 744.2743. M+H, C₃₈H₄₃ClN₇O₅S requires 744.2729. LC/MS (C-18 column, gradient elution 5 minute chromatograph, 95:5 to 5:95 water/acetonitrile, retention time 2.53 min).

### Example 89

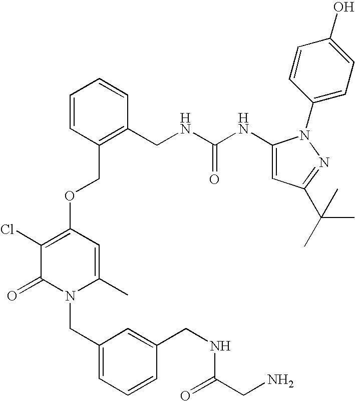

### 1-(2-((1-(3-((2-aminoacetamido)methyl)benzyl)-3-chloro-6-methyl-2-oxo-1,2-dihydropyridin-4-yloxy)methyl)benzyl)-3-(3-tert-butyl-1-(4-hydroxyphenyl)-1H-pyrazol-5-yl)urea

### Step 1: synthesis of the intermediate tert-butyl 2-(3-((4-(2-((3-(3-tert-butyl-1-(4-hydroxyphenyl)-1H-pyrazol-5-yl)ureido)methyl)benzyloxy)-3-chloro-6-methyl-2-oxopyridin-1(2H)-yl)methyl)benzylamino)-2-oxoethylcarbamate

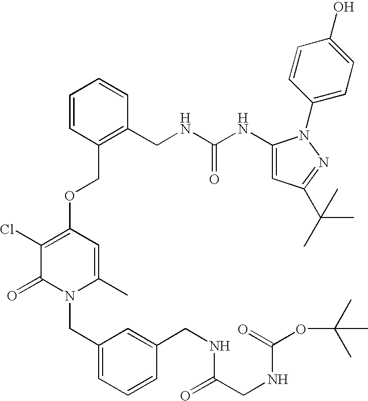

A 100 mL round bottom flask was charged with 4-(5-amino-3-tert-butyl-1H-pyrazol-1-yl)phenol (1.16 g, 5.00 mmol) and methylene chloride (30 mL). To this suspension was added saturated aqueous sodium bicarbonate solution (40 mL) and a commercially available toluene solution of phosgene (Fluka, 20% concentration, 3.0 mL, roughly 5.4 mmol). After 20 minutes the reaction emulsion separated into two layers, and the organic extract was concentrated by nitrogen stream to a residue and then suspended in THF (5.0 mL). To this resulting solution was added a solution of the intermediate tert-butyl 2-(3-((4-(2-(aminomethyl)benzyloxy)-3-chloro-6-methyl-2-oxopyridin-1(2H)-yl)methyl)benzylamino)-2-oxoethylcarbamate (555 mg, 1.00 mmol) in THF (5 mL). After 1 hour, the reaction was concentrated to a residue and was filtered and purified by reverse phase C-18 chromatography using a 15 minute run and water/acetonitrile (95:5 to 5:95) gradient solvent system stabilized with 0.1% TFA. The resulting intermediate compound was obtained as its mono-TFA salt (213 mg, 23%). HRMS (m/z) 812.3512. M+H, C₄₃H₅₁ClN₇O₇ requires 812.3533. LC/MS (C-18 column, gradient elution 5 minute chromatograph, 95:5 to 5:95 water/acetonitrile, retention time 2.78 min).

### Step 2: Preparation of the Title Compound

A 100 mL round bottom flask was charged with the previous intermediate, tert-butyl 2-(3-((4-(2-((3-(3-tert-butyl-1-(4-hydroxyphenyl)-1H-pyrazol-5-yl)ureido)methyl)benzyloxy)-3-chloro-6-methyl-2-oxopyridin-1(2H)-yl)methyl)benzylamino)-2-oxoethylcarbamate (212 mg, 0.229 mmol) and suspended in commercially available HCl dioxane solution (Aldrich, 4 N in 1,4 dioxane, 5 mL, 20 mmol). The reaction suspension was stripped to a residue by nitrogen stream, and the resulting residue was filtered and purified by reverse phase C-18 chromatography using a 15 minute run and water/acetonitrile (95:5 to 5:95) gradient solvent system stabilized with 0.1% TFA. The resulting title compound was obtained as its mono-TFA salt (81 mg, 43%). ¹HNMR (400 MHz, methanol-d4) δ ppm: 1.35 (s, 9H), 2.39 (s, 3H), 3.63 (s, 2H), 4.38 (s, 2H), 4.55 (s, 2H), 5.39 (s, 2H), 5.42 (s, 2H), 6.60 (s, 1H), 7.04 (d, J=8.4 Hz, 2H), 7.10 (s, 1H), 7.20 (d, J=8.4 Hz, 2H), 7.58-7.38 (m, 8H). HRMS (m/z) 712.3014. M+H, C₃₈H₄₃ClN₇O₅ requires 712.3009. LC/MS (C-18 column, gradient elution 5 minute chromatograph, 95:5 to 5:95 water/acetonitrile, retention time 2.27 min).

### Example 90

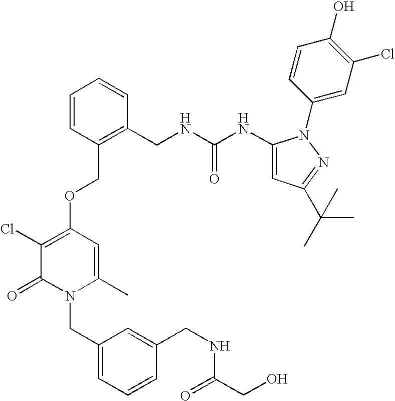

### 1-(2-((1-(3-((2-hydroxyacetamido)methyl)benzyl)-3-chloro-6-methyl-2-oxo-1,2-dihydropyridin-4-yloxy)methyl)benzyl)-3-(3-tert-butyl-1-(3-chloro-4-hydroxyphenyl)-1H-pyrazol-5-yl)urea

### Step 1: Synthesis of the Title Compound

The title compound was prepared in an identical fashion to that of the example 1-(2-((1-(3-((2-aminoacetamido)methyl)benzyl)-3-chloro-6-methyl-2-oxo-1,2-dihydropyridin-4-yloxy)methyl)benzyl)-3-(3-tert-butyl-1-(3-hydroxyphenyl)-1H-pyrazol-5-yl)urea, with two substitutions. First, a substitution at step 6, replacing N-(Boc)-Glycine with glycolic acid (Aldrich product number 124737) on an identical scale. Second, a substitution in step 7 of the reagent 3-(5-amino-3-tert-butyl-1H-pyrazol-1-yl)phenol with 4-(5-amino-3-tert-butyl-1H-pyrazol-1-yl)-2-chlorophenol. The title compound was obtained as its mono-TFA salt (19 mg, 13% yield final step). ¹HNMR (400 MHz, methanol-d4) δ ppm: 1.27 (s, 9H), 2.33 (s, 3H), 3.99 (s, 2H), 4.37 (s, 2H), 4.44 (s, 2H), 5.32 (s, 2H), 5.39 (s, 2H), 6.21 (s, 1H), 6.49 (s, 1H), 6.92 (app d, J=13.0 Hz, 2H), 6.98 (app d, J=8.0 Hz, 1H), 7.03 (s, 1H), 7.35-7.11 (m, 5H), 7.39 (app d, J=2.3 Hz, 1H), 7.42 (app d, J=6.0 Hz, 1H). HRMS (m/z) 747.2447. M+H, C₃₈H₄₁Cl₂N₆O₆ requires 747.2459. LC/MS (C-18 column, gradient elution 5 minute chromatograph, 95:5 to 5:95 water/acetonitrile, retention time 2.79 min).

### Intermediate 48i

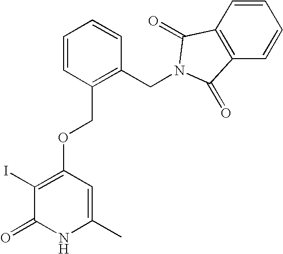

### 2-(2-{[(3-iodo-6-methyl-2-oxo-1,2-dihydropyridin-4-yl)oxy]methyl}benzyl)-1H-isoindole-1,3(2H)-dione

### Step 1: Preparation of 2-[2-(chloromethyl)benzyl]-1H-isoindole-1,3(2H)-dione

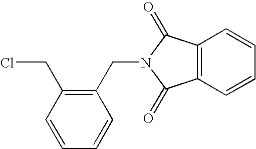

To a 250 mL round bottom was added potassium phthalimide (5.45 g, 29.41 mmol), □□′-dichloro-□-xylene (8.75 g, 50.00 mmol), and N,N-dimethylformamide (117 mL). This reaction mixture was then heated at 50° C. for 24 hours. At this time, the reaction was diluted with ethyl acetate (100 mL) and the solids collected by filtration. The filtrated was then concentrated to afford thick yellow crude oil. The crude oil was then subjected to chromatography (silica gel, ethyl acetate/hexanes) to afford a white solid (4.80 g, 57%). ¹H NMR (400 MHz, DMSO-d₆) δ ppm 7.77-7.91 (5H, m), 7.38-7.48 (1H, m), 7.18-7.32 (2H, m), 4.95 (2H, s), 4.91 (2H, s). LC-MS m/z 308.0 (M+Na calcd for C₁₆H₁₂ClNO₂ requires 308.0).

### Step 2: Preparation of 2-(2-{[(6-methyl-2-oxo-1,2-dihydropyridin-4-yl)oxy]methyl}benzyl)-1H-isoindole-1,3(2H)-dione

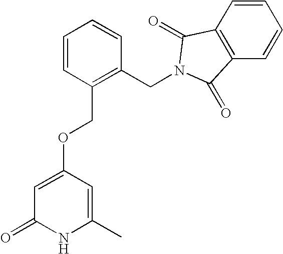

4-hydroxy-6-methylpyridin-2(1H)-one (10.00 g, 79.92 mmol) and 1,8-diazabicyclo[5.4.0]undec-7-ene (12.17 g, 79.92 mmol) were suspended in 1-methyl-2-pyrrolidinone (60 mL). This mixture was then heated at 60° C. and 2-[2-(chloromethyl)benzyl]-1H-isoindole-1,3(2H)-dione (from step 1) (22.83 g, 79.92 mmol) dissolved in 60 mL of 1-methyl-2-pyrrolidinone was added dropwise while maintaining the temperature between 60-70° C. Upon completion of addition, the reaction mixture was heated for 10 hours at 70° C. The reaction was cooled to room temperature, diluted with acetonitrile (80 mL) and the solids collected by filtration. The solids were then rinsed with acetonitrile (2×100 mL) to afford a white solid (14.95 g, 50%). ¹H NMR (400 MHz, DMSO-d₆) δ ppm 11.06 (1H, s), 7.81 (5H, s), 7.10-7.52 (3H, m), 5.66 (1H, s), 5.50 (1H, s), 5.14 (2H, s), 4.80 (2H, s), 2.01 (3H, s). LC-MS m/z 375.0 (M+H calcd for C₂₂H₁₈N₂O₄ requires 375.0).

### Step 3: Preparation of Title Compound

2-(2-{[(6-methyl-2-oxo-1,2-dihydropyridin-4-yl)oxy]methyl}benzyl)-1H-isoindole-1,3(2H)-dione (from step 2) (14.85 g, 39.66 mmol) and N-iodosuccinimide (9.82 g, 43.63 mmol) were suspended in acetonitrile (159 mL). Dichloroacetic acid (0.82 mL) was added and the reaction mixture heated at 65° C. for 1.5 hours. The reaction was cooled to room temperature and the solids collected by filtration. The solids were then rinsed with acetonitrile and vacuum dried. The crude solid was purified by dissolving in hot N,N-dimethylformamide (100 mL), adding hot H₂O (50 mL), cooling to room temperature and collection of solids by filtration. The solids were then rinsed with N,N-dimethylformamide/H₂O (2:1, 2×75 mL). The solids were then vacuum dried to afford an off-white solid (14.23 g, 72%). ¹H NMR (400 MHz, DMSO-d₆) δ ppm 11.68 (1H, s), 7.83 (5H, s), 7.54 (1H, s), 7.14-7.39 (2H, m), 6.26 (1H, s), 5.41 (2H, s), 4.87 (2H, s), 2.18 (3H, s). LC-MS m/z 501.0 (M+H calcd for C₂₂H₁₇IN₂O₄ requires 501.0).

### Intermediate 49i

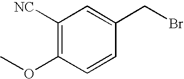

### 5-(bromomethyl)-2-methoxybenzonitrile

### Step 1: Preparation of 5-(hydroxymethyl)-2-methoxybenzonitrile

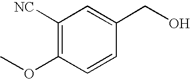

Methyl 3-cyano-4-methoxybenzoate (21.1 g, 110.5 mmol) and calcium borohydride bis tetrahydrofuran complex (52.0 g, 243.1 mmol) were stirred in 2 L tetrahydrofuran at room temperature overnight. The reaction was quenched with 1 L water and extracted 3 times with 500 ml ethyl acetate. The combined organic layers were dried over MgSO₄ and evaporated to a solid (18.3 g, quant.). ¹HNMR (400 MHz, CHLOROFORM-d) δ ppm 3.92 (s, 3H) 4.62 (s, 2H) 6.85-7.01 (m, J=8.32 Hz, 1H) 7.42-7.60 (m, 2H); LC/MS, t₁=1.22 minutes (5 to 95% acetonitrile/water over 5 minutes at 1 ml/min, at 254 nm, at 50° C.), ES-MS m/z 164 (M+H).

### Step 2: Preparation of the title compound

5-(hydroxymethyl)-2-methoxybenzonitrile (from Step 1) (18.2 g, 111.8 mmol) was dissolved in 1 L methylene chloride and cooled to 0° C. with mechanical stirring. A cooled 1.0 M solution of tribromophosphine in methylene chloride (335 ml, 335 mmol) was added dropwise over 25 minutes and stirred for 45 minutes. The reaction was slowly quenched with 500 ml of cold water. An exotherm was seen during the quench. The layers were separated and the organic layer was washed with 500 ml water and 500 ml NaHCO₃ solution, dried over MgSO₄ and evaporated to a solid (22.6 g, 89% yield). ¹H NMR (400 MHz, CHLOROFORM-d) δ ppm 3.93 (s, 3H) 4.42 (s, 2H) 6.81-7.01 (m, J=8.59 Hz, 1H) 7.44-7.64 (m, 2H); LC/MS, tᵣ=2.59 minutes (5 to 95% acetonitrile/water over 5 minutes at 1 ml/min, at 254 nm, at 50° C.), ES-MS m/z 226 (M+H); ES-HRMS m/z 225.9866 (M+H calcd for C₉H₉BrNO requires 225.9862).

### Intermediate 50i

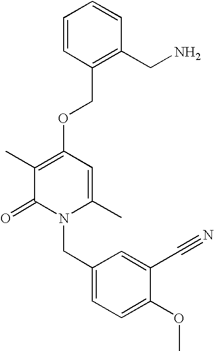

### 5-((4-(2-(aminomethyl)benzyloxy)-3,6-dimethyl-2-oxopyridin-1(2H)-yl)methyl)-2-methoxybenzonitrile

### Step 1: Preparation of 5-((4-(2-((1,3-dioxoisoindolin-2-yl)methyl)benzyloxy)-3-iodo-6-methyl-2-oxopyridin-1(2H)-yl)methyl)-2-methoxybenzonitrile

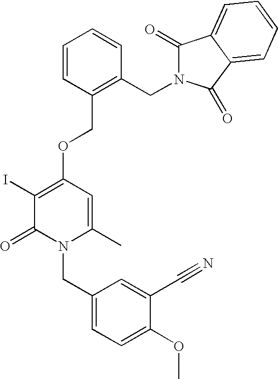

2-(2-((3-iodo-6-methyl-2-oxo-1,2-dihydropyridin-4-yloxy)methyl)benzyl)isoindoline-1,3-dione (14.1 g, 28.2 mmol) was dissolved in 225 ml tetrahydrofuran and cooled to 0° C. 5-(bromomethyl)-2-methoxybenzonitrile (9.6 g, 42.3 mmol) was added, followed by slow addition of 95% NaH (813 mg, 33.9 mmol). The reaction was warmed to 60° C. and heated overnight. At this point, the reaction contained a 3:1 ratio of desired product to O-alkylated product, with <10% starting material. The reaction was quenched with 100 ml water and evaporated to remove the tetrahydrofuran. 200 ml of methylene chloride was added and the reaction extracted. A resulting precipitate was filtered and found to contain starting material. The filtrate was washed with 200 ml NaHCO₃ solution, dried over MgSO₄ and evaporated to a solid. Column silica gel chromatography was performed using 2% methanol in methylene chloride. The resulting oil was triturated with ethyl acetate to obtain a solid (10.2 g, 56% yield). ¹HNMR (400 MHz, CHLOROFORM-d) δ ppm 2.36 (s, 3H) 3.89 (s, 3H) 4.94 (s, 2H) 5.28 (s, 2H) 5.50 (s, 2H) 6.15 (s, 1H) 6.91 (d, J=8.59 Hz, 1H) 7.26-7.40 (m, 3H) 7.45-7.58 (m, 3H) 7.65-7.73 (m, 2H) 7.76-7.87 (m, 2H); LC/MS, tᵣ=3.31 minutes (5 to 95% acetonitrile/water over 5 minutes at 1 ml/min, at 254 nm, at 50° C.), ES-MS m/z 646 (M+H).

### Step 2: Preparation of 5-((4-(2-((1,3-dioxoisoindolin-2-yl)methyl)benzyloxy)-3,6-dimethyl-2-oxopyridin-1(2H)-yl)methyl)-2-methoxybenzonitrile

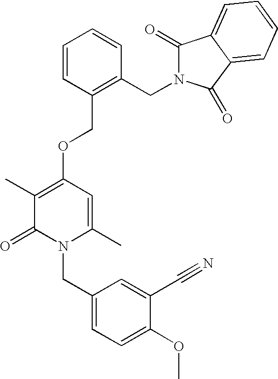

5-((4-(2-((1,3-dioxoisoindolin-2-yl)methyl)benzyloxy)-3-iodo-6-methyl-2-oxopyridin-1(2H)-yl)methyl)-2-methoxybenzonitrile (from Step 1) (10.2 g, 15.9 mmol), tetramethyl tin (4.6 ml, 33.3 mmol), lithium chloride (2.4 g, 55.5 mmol) and dichlorobis(triphenylphosphine)palladium II (1.1 g, 1.6 mmol) were dissolved in 150 ml of N,N-dimethylformamide and refluxed overnight. The reaction was cooled and poured into 1.5 L cold water. The resulting grey solid was filtered. A silica gel column chromatography was performed with 3% methanol in methylene chloride. The crude product was triturated with acetonitrile and washed with ether to give a solid (4.14 g, 49% yield). ¹H NMR (400 MHz, CHLOROFORM-d) δ ppm 1.93 (s, 3H) 2.32 (s, 3H) 3.89 (s, 3H) 4.93 (s, 2H) 5.24 (s, 2H) 5.37 (s, 2H) 6.17 (s, 1H) 6.91 (d, J=8.59 Hz, 1H) 7.27-7.37 (m, 3H) 7.38-7.47 (m, 2H) 7.47-7.53 (m, 1H) 7.66-7.75 (m, 2H) 7.76-7.85 (m, 2H); LC/MS, tᵣ=3.20 minutes (5 to 95% acetonitrile/water over 5 minutes at 1 ml/min, at 254 nm, at 50° C.), ES-MS m/z 534 (M+H).

### Step 3: Preparation of the Title Compound

5-((4-(2-((1,3-dioxoisoindolin-2-yl)methyl)benzyloxy)-3,6-dimethyl-2-oxopyridin-1(2H)-yl)methyl)-2-methoxybenzonitrile (from step 2) (4.1 g, 7.7 mmol) was suspended in 175 ml ethanol. Hydrazine monohydrate (1.8 ml, 37.2 mmol) was added and stirred at room temperature. The reaction was filtered to remove precipitated thalimide bi product. The filtrate was evaporated to a solid. The solid was suspended in 500 ml water and stirred for 30 minutes, filtered and dried to obtain a solid (3.5 g, quant). ¹H NMR (400 MHz, DMSO-d₆) δ ppm 1.86 (s, 3H) 2.25 (s, 3H) 3.80 (s, 2H) 3.85 (s, 3H) 5.18 (s, 2H) 5.23 (s, 2H) 6.38 (s, 1H) 7.17 (s, 1H) 7.20 (s, 1H) 7.20-7.27 (m, 1H) 7.27-7.34 (m, 1H) 7.37-7.50 (m, 3H) 7.83 (dd, J=6.04, 3.36 Hz, 1H) 8.04 (dd, J=6.04, 3.36 Hz, 1H); LC/MS, tᵣ=2.08 minutes (5 to 95% acetonitrile/water over 5 minutes at 1 ml/min, at 254 nm, at 50° C.), ES-MS m/z 404 (M+H); ES-HRMS m/z 404.1971 (M+H calcd for C24H26N3O3 requires 404.1969).

### Example 91

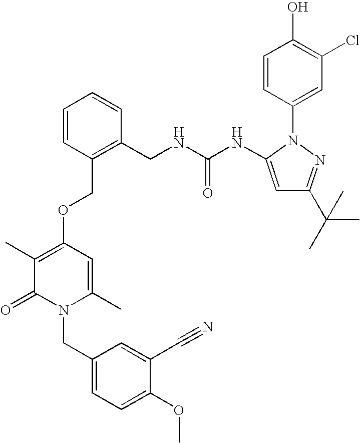

### 1-(2-((1-(3-cyano-4-methoxybenzyl)-3,6-dimethyl-2-oxo-1,2-dihydropyridin-4-yloxy)methyl)benzyl)-3-(3-tert-butyl-1-(3-chloro-4-hydroxyphenyl)-1H-pyrazol-5-yl)urea

5-((4-(2-(aminomethyl)benzyloxy)-3,6-dimethyl-2-oxopyridin-1(2H)-yl)methyl)-2-methoxybenzonitrile (from above) (500 mg, 1.2 mmol) was dissolved in 15 ml tetrahydrofuran. Phenyl 3-tert-butyl-1-(4-(tert-butyldimethylsilyloxy)-3-chlorophenyl)-1H-pyrazol-5-ylcarbamate (620 mg, 1.2 mmol) and 1 ml triethylamine were added and refluxed. After 4 hours, the reaction was cooled to room temperature and stirred for 48 hours. LC-MS indicated that the TBS group had been deprotected during the reaction. The reaction was diluted with 50 ml ethyl acetate and washed with 50 ml of 2.5N NaOH, and 50 ml water. The organic layer was dried over MgSO₄ and evaporated. The resulting oil was ran on a series of silica gel preparative plates using 5% methanol in methylene chloride. The resulting oil was triturated with ether to yield a white solid (111 mg, 13% yield). ¹H NMR (400 MHz, CHLOROFORM-d) δ ppm 1.27 (s, 9H) 1.75 (s, 3H) 2.23 (s, 3H) 3.83 (s, 3H) 4.43 (d, J=5.64 Hz, 2H) 4.96 (s, 2H) 5.13 (s, 2H) 6.14 (s, 1H) 6.32 (s, 1H) 6.45 (d, J=8.59 Hz, 1H) 6.64-6.73 (m, 1H) 6.80 (d, J=8.86 Hz, 2H) 7.11 (d, J=2.15 Hz, 1H) 7.17-7.30 (m, 5H) 7.37 (m, 2H) 7.73-7.84 (m, 1H); LC/MS, tᵣ=3.90 minutes (5 to 95% acetonitrile/water over 5 minutes at 1 ml/min, at 254 nm, at 50° C.), ES-MS m/z 695 (M+H); ES-HRMS m/z 695.2713 (M+H calcd for C38H40ClN6O5 requires 695.2743).

### Example 92

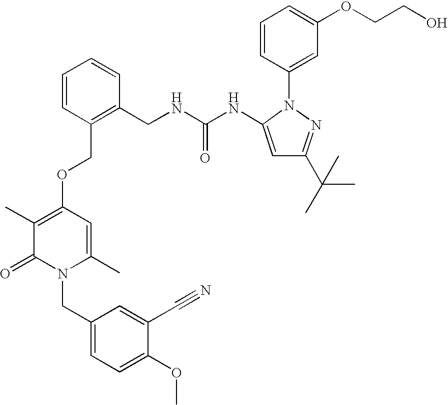

### 1-(2-((1-(3-cyano-4-methoxybenzyl)-3,6-dimethyl-2-oxo-1,2-dihydropyridin-4-yloxy)methyl)benzyl)-3-(3-tert-butyl-1-(3-(2-hydroxyethoxy)phenyl)-1H-pyrazol-5-yl)urea

5-((4-(2-(aminomethyl)benzyloxy)-3,6-dimethyl-2-oxopyridin-1(2H)-yl)methyl)-2-methoxybenzonitrile (from above) (500 mg, 1.2 mmol) was dissolved in 15 ml tetrahydrofuran. Phenyl 3-tert-butyl-1-(3-(2-(tetrahydro-2H-pyran-2-yloxy)ethoxy)phenyl)-1H-pyrazol-5-ylcarbamate (595 mg, 1.2 mmol) and 1 ml pyridine were added and stirred at reflux for 4 hours, then at room temperature overnight. The reaction was diluted with 50 ml ethyl acetate and washed with 50 ml of 2.5N NaOH, and 50 ml water. The organic layer was dried over MgSO₄ and evaporated. The resulting oil was dissolved in 10 ml methanol. P-Toluene sulphonic acid monohydrate (18 mg, 0.6 mmol) was added and stirred at room temperature overnight. Deprotection was monitored by TLC. The reaction was diluted with 50 ml ethyl acetate and washed with 50 ml of 2.5N NaOH, and 50 ml water. The organic layer was dried over MgSO₄ and evaporated. The resulting oil was ran on a series of silica gel preparative plates using 5% methanol in methylene chloride to obtain both the desired compound and some of the THP protected product. The desired compound was obtained as an oil, which was triturated with ether to yield a white solid (111 mg, 13% yield). ¹H NMR (400 MHz, CHLOROFORM-d) δ ppm 1.19 (t, J=7.12 Hz, 2H) 1.29 (s, 9H) 1.80 (s, 3H) 2.20 (s, 3H) 3.46 (q, J=6.98 Hz, 2H) 3.71 (s, 2H) 3.79 (d, J=4.57 Hz, 2H) 3.85 (s, 3H) 4.43 (d, J=5.37 Hz, 2H) 4.99 (s, 1H) 5.08 (s, 2H) 6.08 (s, 1H) 6.22 (s, 1H) 6.38 (s, 1H) 6.59 (d, J=8.32 Hz, 1H) 6.77-6.85 (m, 2H) 6.91 (d, J=7.25 Hz, 1H) 7.09 (t, J=8.06 Hz, 1H) 7.19 (d, J=2.15 Hz, 1H) 7.25-7.32 (m, 3H) 7.40 (dd, J=6.58, 2.01 Hz, 1H); LC/MS, tᵣ=3.05 minutes (5 to 95% acetonitrile/water over 5 minutes at 1 ml/min, at 254 nm, at 50° C.), ES-MS m/z 705 (M+H); ES-HRMS m/z 705.3401 (M+H calcd for C40H45N6O6 requires 705.3395).

### Example 93

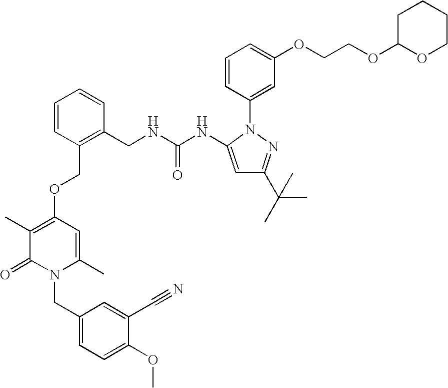

### 1-(2-((1-(3-cyano-4-methoxybenzyl)-3,6-dimethyl-2-oxo-1,2-dihydropyridin-4-yloxy)methyl)benzyl)-3-(3-tert-butyl-1-(3-(2-(tetrahydro-2H-pyran-2-yloxy)ethoxy)phenyl)-1H-pyrazol-5-yl)urea

The title compound was isolated as an intermediate from the series of silica gel preparative plates ran to isolate above, as a solid (309.4 mg, 31% yield). ¹H NMR (400 MHz, CHLOROFORM-d) 3 ppm 1.28 (s, 9H) 1.40-1.56 (m, 4H) 1.62 (s, 1H) 1.64-1.70 (m, 1H) 1.77 (d, J=5.91 Hz, 1H) 1.88 (s, 3H) 2.22 (s, 3H) 3.41-3.50 (m, 1H) 3.68-3.76 (m, 1H) 3.81 (dd, J=11.01, 7.79 Hz, 1H) 3.86 (s, 3H) 3.94-4.01 (m, 1H) 4.03-4.09 (m, 1H) 4.43 (d, J=5.64 Hz, 2H) 4.59 (t, J=3.63 Hz, 1H) 5.00 (s, 2H) 5.09 (s, 2H) 6.05 (s, 1H) 6.11 (t, J=5.37 Hz, 1H) 6.31 (s, 1H) 6.74 (dd, J=8.32, 1.88 Hz, 1H) 6.82 (d, J=8.59 Hz, 1H) 6.92 (d, J=7.79 Hz, 1H) 6.97 (t, J=2.15 Hz, 1H) 7.06 (s, 1H) 7.12 (t, J=8.06 Hz, 1H) 7.22 (d, J=2.15 Hz, 2H) 7.23-7.26 (m, 1H) 7.26-7.30 (m, 2H) 7.37-7.42 (m, 1H); LC/MS, tᵣ=3.67 minutes (5 to 95% acetonitrile/water over 5 minutes at 1 ml/min, at 254 nm, at 50° C.), ES-MS m/z 789 (M+H); ES-HRMS m/z 789.3978 (M+H calcd for C45H53N6O7 requires 789.3970).

### Example 94

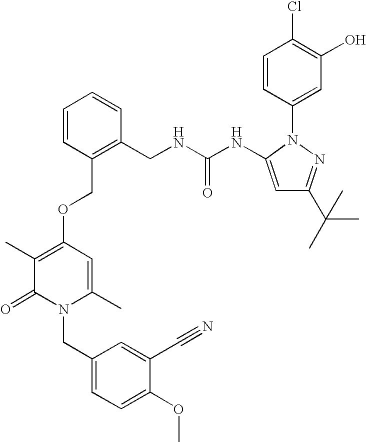

### 1-(2-((1-(3-cyano-4-methoxybenzyl)-3,6-dimethyl-2-oxo-1,2-dihydropyridin-4-yloxy)methyl)benzyl)-3-(3-tert-butyl-1-(4-chloro-3-hydroxyphenyl)-1H-pyrazol-5-yl)urea

5-((4-(2-(aminomethyl)benzyoxy)-3,6-dimethyl-2-oxopyridin-1(2H)-yl)methyl)-2-methoxybenzonitrile (500 mg, 1.2 mmol) was dissolved in 15 ml tetrahydrofuran. Phenyl 3-tert-butyl-1-(3-(tert-butyldimethylsilyloxy)-4-chlorophenyl)-1H-pyrazol-5-ylcarbamate (620 mg, 1.2 mmol) and 1 ml triethylamine were added and refluxed. After 4 hours, the reaction was cooled to room temperature and stirred overnight. LC-MS indicated that the TBS group had been deprotected during the reaction. The reaction was diluted with 50 ml ethyl acetate and washed with 50 ml of 2.5N NaOH, and 50 ml water. The organic layer was dried over MgSO₄ and evaporated. The resulting oil was ran on a series of silica gel preparative plates using 5% methanol in methylene chloride. The resulting oil was triturated with ether to yield a white solid (207 mg, 24% yield). ¹H NMR (400 MHz, CHLOROFORM-d) δ ppm 1.27 (s, 9H) 1.76 (s, 3H) 2.22 (s, 3H) 3.82 (s, 3H) 4.40 (s, 2H) 4.93 (s, 2H) 5.08 (s, 2H) 6.11 (s, 1H) 6.38 (s, 1H) 6.61 (s, 1H) 6.77 (d, J=8.86 Hz, 2H) 6.92 (s, 1H) 7.05-7.22 (m, 7H) 7.37 (d, J=7.25 Hz, 1H) 7.95 (s, 1H); LC/MS, tᵣ=3.31 minutes (5 to 95% acetonitrile/water over 5 minutes at 1 ml/min, at 254 nm, at 50° C.), ES-MS m/z 695 (M+H); ES-HRMS m/z 695.2749 (M+H calcd for C₃₈H₄₀ClN₆O₅ requires 695.2743).

### Example 95

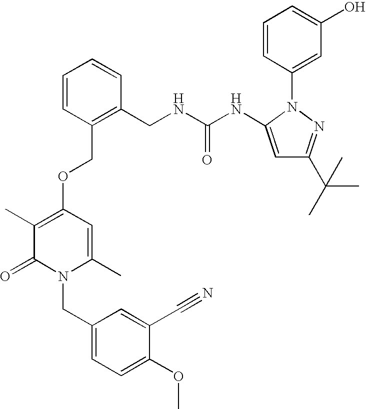

### 1-(2-((1-(3-cyano-4-methoxybenzyl)-3,6-dimethyl-2-oxo-1,2-dihydropyridin-4-yloxy)methyl)benzyl)-3-(3-tert-butyl-1-(3-hydroxyphenyl)-1H-pyrazol-5-yl)urea

### Step 1: Preparation of phenyl 3-tert-butyl-1-(3-(tert-butyldimethylsilyloxy)phenyl)-1H-pyrazol-5-ylcarbamate

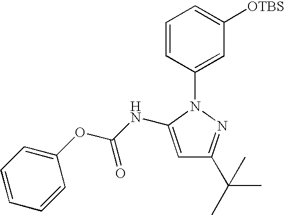

3-tert-butyl-1-(3-(tert-butyldimethylsilyloxy)phenyl)-1H-pyrazol-5-amine (876 mg, 2.5 mmol) was dissolved in 50 ml tetrahydrofuran and cooled to 0° C. Pyridine (0.27 ml, 3.3 mmol) was added, followed by dropwise addition of phenylchloroformate (0.54 ml, 4.3 mmol). The reaction was stirred at 0° C. for 10 minutes, then allowed to warm to room temperature for 1 hour. The reaction was then diluted with 100 ml of ethyl acetate and washed with 100 ml water and 100 ml brine. The organic layer was dried over MgSO₄ and evaporated to a solid (1.1 g, 95% yield). ¹H NMR (400 MHz, CHLOROFORM-d) δ ppm 0.23 (s, 6H) 0.99 (s, 9H) 1.33 (s, 9H) 6.47 (s, 1H) 6.87 (dd, J=8.19, 2.28 Hz, 1H) 6.97 (s, 1H) 7.07-7.17 (m, 3H) 7.22-7.26 (m, 1H) 7.36 (q, J=7.61 Hz, 4H); LC/MS, tᵣ=4.69 minutes (5 to 95% acetonitrile/water over 5 minutes at 1 ml/min, at 254 nm, at 50° C.), ES-MS m/z 466 (M+H); ES-HRMS m/z 466.2521 (M+H calcd for C26H36N3O3Si requires 466.2520).

### Step 2: Preparation of the Title Compound

Phenyl 3-tert-butyl-1-(3-(tert-butyldimethylsilyloxy)phenyl)-1H-pyrazol-5-ylcarbamate (from Step 1) (578 mg, 1.2 mmol) and 5-((4-(2-(aminomethyl)benzyloxy)-3,6-dimethyl-2-oxopyridin-1(2H)-yl)methyl)-2-methoxybenzonitrile (500 mg, 1.2 mmol) were dissolved in 15 ml tetrahydrofuran and 1 ml trimethylamine and stirred at reflux. After 1 hour, 1M t-butylammonium fluoride in tetrahydrofuran (1.2 ml, 1.2 mmol) was added and stirred at room temperature for 1 hour. The reaction was then diluted with 50 ml ethyl acetate and washed with 50 ml of 2.5N NaOH solution and 50 ml water. The organic layer was dried over MgSO₄ and evaporated. The resulting oil was ran on a series of silica gel preparative plates using 6% methanol in methylene chloride. The resulting oil was triturated with ether to yield a white solid (279 mg, 34% yield). ¹H NMR (400 MHz, DMSO-d₆) δ ppm 1.20 (s, 9H) 1.87 (s, 3H) 2.23 (s, 3H) 3.85 (s, 3H) 4.32 (d, J=5.64 Hz, 2H) 5.17 (s, 2H) 5.20 (s, 2H) 6.21 (s, 1H) 6.35 (s, 1H) 6.73 (d, J=9.40 Hz, 1H) 6.83 (s, 1H) 6.85 (s, 1H) 6.98 (t, J=5.64 Hz, 1H) 7.14-7.24 (m, 2H) 7.24-7.34 (m, 3H) 7.35-7.44 (m, 2H) 7.47 (s, 1H) 8.22 (s, 1H) 9.72 (s, 1H); LC/MS, tᵣ=3.09 minutes (5 to 95% acetonitrile/water over 5 minutes at 1 ml/min, at 254 nm, at 50° C.), ES-MS m/z 661 (M+H); ES-HRMS m/z 661.3134 (M+H calcd for C38H41N6O5 requires 661.3133).

### Example 96

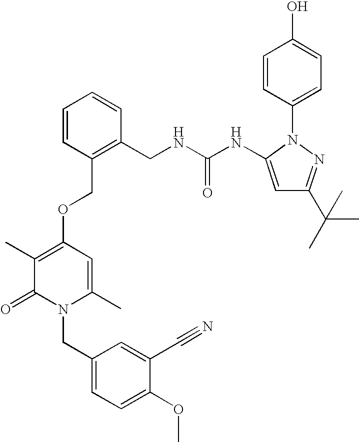

### 1-(2-((1-(3-cyano-4-methoxybenzyl)-3,6-dimethyl-2-oxo-1,2-dihydropyridin-4-yloxy)methyl)benzyl)-3-(3-tert-butyl-1-(4-hydroxyphenyl)-1H-pyrazol-5-yl)urea

5-((4-(2-(aminomethyl)benzyloxy)-3,6-dimethyl-2-oxopyridin-1(2H)-yl)methyl)-2-methoxybenzonitrile (670 mg, 1.7 mmol) was dissolved in 15 ml tetrahydrofuran. Phenyl 3-tert-butyl-1-(4-(tert-butyldimethylsilyloxy)phenyl)-1H-pyrazol-5-ylcarbamate (774 mg, 1.7 mmol) and 1 ml triethylamine were added and stirred at reflux. After 1 hour, 1M t-butylammonium fluoride in tetrahydrofuran (1.7 ml, 1.7 mmol) was added and stirred at room temperature for 30 minutes. The reaction was then diluted with 50 ml ethyl acetate and washed with 50 ml of 2.5N NaOH solution and 50 ml water. The organic layer was dried over MgSO₄ and evaporated. The resulting oil was ran on a series of silica gel preparative plates using 6% methanol in methylene chloride. The resulting oil was triturated with ether to yield a white solid (377 mg, 34% yield). ¹H NMR (400 MHz, DMSO-d₆) δ ppm 1.19 (s, 9H) 1.87 (s, 3H) 2.23 (s, 3H) 3.85 (s, 3H) 4.30 (d, J=5.64 Hz, 2H) 5.17 (s, 2H) 5.20 (s, 2H) 6.17 (s, 1H) 6.34 (s, 1H) 6.80 (d, J=8.86 Hz, 2H) 6.94 (t, J=5.91 Hz, 1H) 7.15-7.21 (m, 3H) 7.22-7.34 (m, 3H) 7.36-7.45 (m, 2H) 7.47 (d, J=2.15 Hz, 1H) 8.05 (s, 1H) 9.68 (s, 1H); LC/MS, tᵣ=2.96 minutes (5 to 95% acetonitrile/water over 5 minutes at 1 ml/min, at 254 nm, at 50° C.), ES-MS m/z 661 (M+H); ES-HRMS m/z 661.3116 (M+H calcd for C38H41N6O5 requires 661.3133).

### Example 97

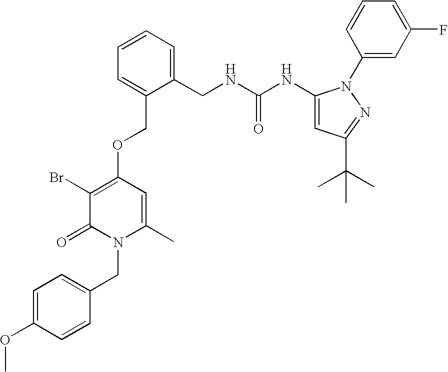

### 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea

### Step 1: Preparation of 2-{[(6-methyl-2-oxo-1,2-dihydropyridin-4-yl)oxy]methyl}benzonitrile

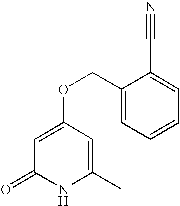

A stirred mixture of 5 g (40 mmol) of 4-hydroxy-6-methyl-2-pyridinone and potassium carbonate (8.28 g, 44 mmol) in dimethylformamide (65 mL) at 65 degrees Celsius was treated with portion wise addition a of 8.63 g (60 mmol) of alpha-bromotolunitrile over 20 minutes. The mixture was allowed to stir for two hours at 65 degrees, then cooled to room temperature and stirred overnight. Then the mixture was slowly poured into 300 mL of ice water resulting in the precipitation of a tan solid, which was collected by vacuum filtration and washed with water (2×100 mL) and hexane (2×100 mL). The solid was dried in vacuo overnight, yielding 6.33 g of the intermediate benzonitrile as a tan solid: LC/MS on 4.6×50 mm C-18 column, tᵣ=1.63 minutes (10 to 90% acetonitrile/water over 5 minutes at 4 ml/min with detection 220 nm, at 30° C.); ES-MS m/z 241 (M+H).

### Step 2: Preparation of 2-({[1-(4-methoxybenzyl)-6-methyl-2-oxo-1,2-dihydropyridin-4-yl]oxy}methyl)benzonitrile

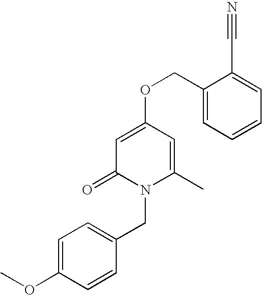

To a stirred mixture of 1.05 g (26.2 mmol) of 60% sodium hydride oil dispersion in 50 mL of dimethylformamide was added 6.3 g (26.2 mmol) of the pyridinone from Step 1 portionwise over 15 minutes. The addition was accompanied by vigorous off-gassing. After complete addition, the flask was warmed to room temperature and stirred for 2 hours. The flask was immersed in an ice bath, and 3.6 mL of para-methoxybenzyl chloride (26.2 mmol) was added dropwise over 20 minutes, then the ice bath was removed. After one hour, the mixture was heated to 65 degrees Celsius and stirred for 15 hours. The flask was cooled to room temperature, and the reaction mixture was slowly poured into 500 mL ice water, resulting in a tan gummy solid. The gummy solid was collected by vacuum filtration and dissolved 200 mL methylene chloride. The organic layer was washed with water (1×500 mL), dried over anhydrous MgSO₄, filtered and concentrated in vacuo to afford 11.55 g of a tan gummy solid. A portion of the crude product was purified by normal phase flash column chromatography on a 70 g silica gel column (25-75% ethyl acetate/methylene chloride gradient). Pure fractions were pooled and concentrated in vacuo to yield 1.61 g of 2-({[1-(4-methoxybenzyl)-6-methyl-2-oxo-1,2-dihydropyridin-4-yl]oxy}methyl)benzonitrile as an off-white solid. ¹H NMR (400 MHz, d₃-CH₃Cl) δ 2.23 (s, 3H), 3.76 (s, 3H), 5.17 (s, 2H), 5.20 (s, 2H), 5.84 (d, J=2.0 Hz, 1H), 5.97 (d, J=2.8 Hz, 1H), 6.82, d, J=8.8 Hz, 2H), 7.09 (d, J=8.4 Hz, 2H), 7.45 (td, J=7.4, 1.2 Hz, 1H), 7.56 (d, J=7.2 Hz, 1H), 7.62 (td, J=7.6, 1.2 Hz, 1H), 7.71 (d, J=7.6 Hz, 1H); LC/MS on 4.6×50 mm C-18 column, tᵣ=2.55 minutes (10 to 90% acetonitrile/water over 5 minutes at 4 ml/min with detection 220 nm, at 30° C.); ES-MS m/z 361 (M+H).

### Step 3: Preparation of 2-({[3-bromo-1-(4-methoxybenzyl)-6-methyl-2-oxo-1,2-dihydropyridin-4-yl]oxy}methyl)benzonitrile

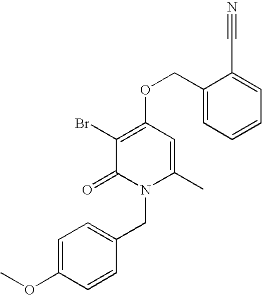

To a stirred solution of 1.57 g of tert-butyl {3-[7-({[3,5-bis(trifluoromethyl)benzyl](2-methyl-2H-tetrazol-5-yl)amino}methyl)-1-methyl-5-(trifluoromethyl)-1H-benzimidazol-2-yl]phenoxy}acetate (4.37 mmol) in 9 mL of anhydrous acetonitrile at 0 degrees Celsius was added 0.78 g of N-bromosuccinimide (4.37 mmol) in one portion. The mixture was stirred with cooling for one hour then the ice bath was removed. After one hour, and addition 0.19 g of N-bromosuccinimide was added, and the mixture stirred an additional hour. The mixture was treated with 5 mL of 10% sodium sulfite for one hour, then the reaction mixture was partitioned between 100 mL ethyl acetate and 100 mL water. The organic phase was separated, washed with 100 mL brine, dried over magnesium sulfate and concentrated in vacuo to give 2.2 of crude product as a yellow solid. Recrystallization from isopropanol yielded 1.63 g of 2-({[3-bromo-1-(4-methoxybenzyl)-6-methyl-2-oxo-1,2-dihydropyridin-4-yl]oxy}methyl)benzonitrile as a pale yellow solid: ¹H NMR (400 MHz, d₃-CH₃Cl) δ 2.33 (s, 3H), 3.76 (s, 3H), 5.29 (s, 2H), 5.37 (s, 2H), 5.99 (s, 1H), 6.82 (m, 2H), 7.14 (d, J=8.8 Hz, 2H), 7.45 (t, J=7.6 Hz, 1H), 7.69 (m, 2H), 7.85 (d, J=8.4 Hz, 1H); LC/MS on 4.6×50 mm C-18 column, tᵣ=2.65 minutes (10 to 90% acetonitrile/water over 5 minutes at 4 ml/min with detection 220 nm, at 30° C.); ES-MS m/z 439 (M+H).

### Step 4: Preparation of 4-{[2-(aminomethyl)benzyl]oxy}-3-bromo-1-(4-methoxybenzyl)-6-methylpyridin-2(1H)-one

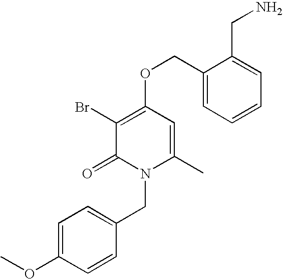

To a stirred solution of 1.62 g of 2-({[3-bromo-1-(4-methoxybenzyl)-6-methyl-2-oxo-1,2-dihydropyridin-4-yl]oxy}methyl)benzonitrile (3.68 mmol) in 7.5 anhydrous tetrahydrofuran at 0 degrees Celsius was added 7.4 mL of 1.0 M solution of borane-tetrahydrofuran complex in tetrahydrofuran (7.4 mmol) dropwise over 10 minutes. The addition was accompanied by vigorous off-gassing. After complete addition, the reaction mixture was warmed to room temperature and stirred for 4 hours. Then the flask was immersed in an ice bath, and 2 mL of methanol was carefully added dropwise to the mixture. The addition was again accompanied by vigorous off-gassing. The mixture was warmed to room temperature and volatiles were removed in vacuo leaving 1.84 g of crude product. The crude product was dissolved in a mixture of 30 mL methanol and 30 mL methylene chloride in a 150 mL wide mouth jar, and 15 g of polymer-bound sulfonic acid (60 mmol) was added. The jar was capped, and the mixture was agitated on a tabletop shaker for 1 hour. The resin was filtered and washed successively with methanol (3×50 mL) and methylene chloride (3×50 mL). The resin was transferred back to the jar and 30 mL of methylene chloride and 30 mL of 7 N ammonia in methanol was added. The mixture was agitated for 1 hour on the tabletop shaker, then the resin was filtered and washed with successively with methanol (2×50 mL) and methylene chloride (2×50 mL). The resin was retreated with 30 mL of methylene chloride and 30 mL of 7 N ammonia in methanol for 2 hour on the tabletop shaker, and the resin was again filtered and washed with successively with methanol (2×50 mL) and methylene chloride (2×50 mL). All filtrates were combined and concentrated in vacuo to yield 1.25 g of product as a tan solid; ¹H NMR (400 MHz, d₃-CH₃Cl) δ 2.29 (s, 3H), 3.76 (s, 3H), 3.93 (s, 2H), 5.27 (s, 4H), 5.37 (s, 2H), 5.99 (s, 1H), 6.82 (d, J=8.8 Hz, 2H), 7.14 (d, J=8.8 Hz, 2H), 7.45 (t, J=7.6 Hz, 1H), 7.69 (m, 2H), 7.85 (d, J=8.4 Hz, 1H); LC/MS on 4.6×50 mm C-18 column, tᵣ=1.58 minutes (10 to 90% acetonitrile/water over 5 minutes at 4 ml/min with detection 220 nm, at 30° C.); ES-MS m/z 443 (M+H).

### Step 5: Preparation of 0.2 M solution of 4-nitrophenyl [3-tert-butyl-1-(3-fluorophenyl)-1H-pyrazol-5-yl]carbamate in methylene chloride

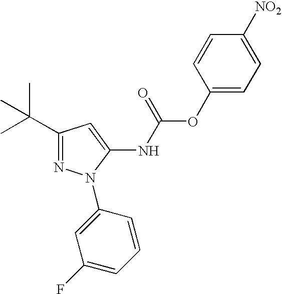

To a solution of 467 mg of 5-amino-3-tert-butyl-1-(3-fluorophenyl)-1H-pyrazole (2 mmol) and 162 uL pyridine (2 mmol) in 4 mL of chloroform was added 3.4 mL of a 0.6 M solution ofpara-nitrochloroformate in chloroform (2 mmol). The mixture was stirred at room temperature for 6 hours, then concentrated in vacuo. LC/MS on 4.6×50 mm C-18 column, tᵣ=3.29 minutes (10 to 90% acetonitrile/water over 5 minutes at 4 ml/min with detection 220 nm, at 30° C.); ES-MS m/z 399 (M+H). The mixture was diluted up to 10 mL volumetrically with methylene chloride (theoretical concentration=0.2 M) and stored in a refrigerator for up to I week without significant degradation as judged by LC/MS.

### Step 6: Preparation of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea

To a stirred solution of the 50 mg amine from step 4 above (0.11 mmol) and 24 uL of triethylamine (0.17 mmol) in 1 mL of methylene chloride was added 0.56 mL of the 0.2 M solution of 4-nitrophenyl [3-tert-butyl-1-(3-fluorophenyl)-1H-pyrazol-5-yl]carbamate described in step 5 above (0.11 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 14 mg of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea as a white solid (18%): ¹H NMR (400 MHz, DMSO-d6) δ 8.47 (s, 1H), 8.37 (s, 1H), 7.43-7.49 (m, 2H), 7.34 (d, J=8.8 Hz, 2H), 7.25-7.32 (m, 3H), 7.15 (m, 1H), 7.04 (d, J=8.8, 2H), 6.86 (d, J=8.8 Hz, 1H), 6.47 (s, 1H), 6.24 (s, 1H), 5.30 (s, 2H), 5.20 (s, 2H), 4.32 (d, J=5.2 Hz, 2H), 3.69 (s, 3H), 2.26 (s, 3H), 1.21 (s, 9H); LC/MS on 4.6×50 mm C-18 column, tᵣ=3.26 minutes (10 to 90% acetonitrile/water over 5 minutes at 4 ml/min with detection 220 nm, at 30° C.); ES-MS m/z 702 (M+H).

### Example 98

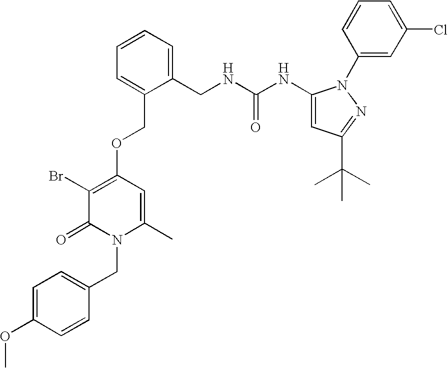

### 1-[2-({[3-bromo-1-(4-methoxybenzyl)-6-methyl-2-oxo-1,2-dihydropyridin-4-yl]oxy}methyl)benzyl]-3-[3-tert-butyl-1-(3-chlorophenyl)-1H-pyrazol-5-yl urea

### Step 1: Preparation of 0.2 M Solution of 4-nitrophenyl [3-tert-butyl-1-(3-chlorophenyl)-1H-pyrazol-5-yl]carbamate in methylene chloride

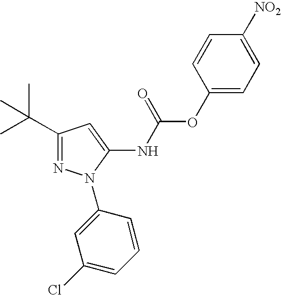

A solution of 500 mg of 5-amino-3-tert-butyl-1-(3-chlorophenyl)-1H-pyrazole (2 mmol) was treated as described in part 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea to obtain a 0.2 M solution of 4-nitrophenyl [3-tert-butyl-1-(3-chlorophenyl)-1H-pyrazol-5-yl]carbamate in methylene chloride. LC/MS on 4.6×50 mm C-18 column, tᵣ=3.46 minutes (10 to 90% acetonitrile/water over 5 minutes at 4 ml/min with detection 220 nm, at 30° C.); ES-MS m/z 415 (M+H).

### Step 2: Preparation of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-chlorophenyl)-1H-pyrazol-5-yl]urea

To a stirred solution of the 50 mg amine from step 4 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea above (0.11 mmol) and 24 uL of triethylamine (0.17 mmol) in 1 mL of methylene chloride was added 0.56 mL of the 0.2 M solution of 4-nitrophenyl [3-tert-butyl-1-(3-chlorophenyl)-1H-pyrazol-5-yl]carbamate described in step 1 above (0.11 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 10 mg of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-chlorophenyl)-1H-pyrazol-5-yl]urea as a white solid (12%): ¹HNMR (400 MHz, DMSO-d₆) δ 8.38 (s, 1H), 7.56 (s, 1H), 7.24-7.47 (m, 7H), 7.03 (d, J=8.8 Hz, 1H), 6.84-6.88 (m, 2H), 6.23 (s, 1H), 5.30 (s, 2H), 5.20 (s, 2H), 4.31 (d, J=6 Hz, 2H), 3.69 (s, 3H), 2.25 (s, 3H), 1.21 (s, 9H); LC/MS on 4.6×50 mm C-18 column, tᵣ=3.40 minutes (10 to 90% acetonitrile/water over 5 minutes at 4 ml/min with detection 220 nm, at 30° C.); ES-MS m/z 718 (M+H).

### Example 99

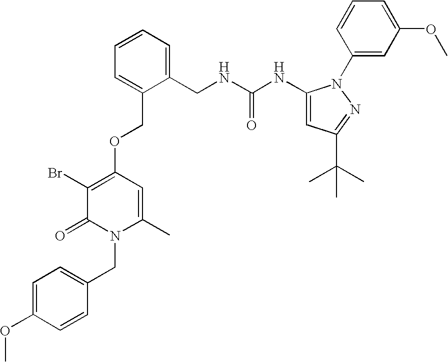

### 1-[2-({[3-bromo-1-(4-methoxybenzyl)-6-methyl-2-oxo-1,2-dihydropyridin-4-yl]oxy}methyl)benzyl]-3-[3-tert-butyl-1-(3-methoxyphenyl)-1H-pyrazol-5-yl]urea

### Step 1: Preparation of 0.2 M solution of 4-nitrophenyl [3-tert-butyl-1-(3-methoxyphenyl)-1H-pyrazol-5-yl]carbamate in methylene chloride

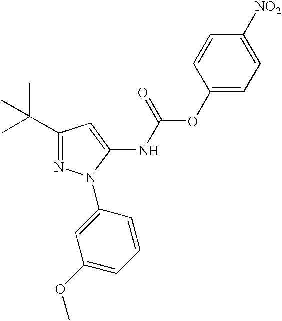

A solution of 491 mg of 3-tert-butyl-1-(3-methoxyphenyl)-1H-pyrazol-5-amine (2 mmol) was treated as described in part 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea to obtain a 0.2 M solution of 4-nitrophenyl [3-tert-butyl-1-(3-methoxyphenyl)-1H-pyrazol-5-yl]carbamate in methylene chloride. LC/MS on 4.6×50 mm C-18 column, tᵣ=3.22 minutes (10 to 90% acetonitrile/water over 5 minutes at 4 ml/min with detection 220 nm, at 30° C.); ES-MS m/z 411 (M+H).

### Step 2: Preparation of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-methoxyphenyl)-1H-pyrazol-5-yl]urea

To a stirred solution of the 50 mg amine from step 4 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea above (0.11 mmol) and 24 uL of triethylamine (0.17 mmol) in 1 mL of methylene chloride was added 0.56 mL of the 0.2 M solution of 4-nitrophenyl [3-tert-butyl-1-(3-methoxyphenyl)-1H-pyrazol-5-yl]carbamate described in step 1 above (0.11 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 28 mg of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-methoxyphenyl)-1H-pyrazol-5-yl]urea as a white solid (34%): ¹H NMR (400 MHz, DMSO-d₆) δ 8.26 (s, 1H), 7.44-7.47 (m, 1H), 7.27-7.36 (m, 4H), 7.00-7.03 (m, 5H), 6.88-6.92 (m, 1H), 6.86 (d, J=8.8 Hz, 2H), 6.46 (s, 1H), 6.22 (s, 1H), 5.30 (s, 2H), 5.20 (s, 2H), 4.32 (d, J=6 Hz, 2H), 3.73 (s, 3H), 3.69 (s, 3H), 2.26 (s, 3H), 1.21 (s, 9H); LC/MS on 4.6×50 mm C-18 column, tᵣ=3.19 minutes (10 to 90% acetonitrile/water over 5 minutes at 4 ml/min with detection 220 nm, at 30° C.); ES-MS m/z 714 (M+H).

### Example 100

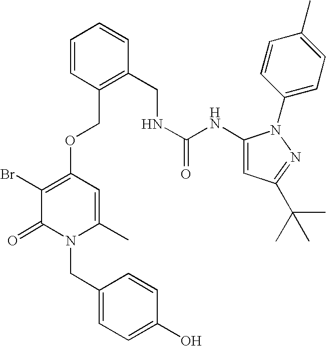

### 1-[2-({[3-bromo-1-(4-hydroxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea

To a solution of 85 mg of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-methoxyphenyl)-1H-pyrazol-5-yl]urea (0.12 mmol) in 1 mL methylene chloride was added 0.37 mL of 1 M boron tribromide in methylene chloride (0.37 mmol). The reaction was stirred for 45 minutes at room temperature, then volatiles were removed in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 16 mg of 1-[2-({[3-bromo-1-(4-hydroxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea as a white solid (19%): ¹H NMR (400 MHz, DMSO-d₆) δ 7.43-7.47 (m, 1H), 7.24-7.34 (m, 5H), 7.24 (t, J=8.4, 2H), 6.98 (m, 1H), 6.92 (d, J=8.4 Hz, 2H), 6.67 (d, J=8.4 Hz, 2H), 6.45 (s, 1H), 6.20 (s, 1H), 5.29 (s, 2H), 5.15 (s, 2H), 4.31 (d, J=5.6 Hz, 2H), 2.31 (s, 3H), 2.26 (s, 3H), 1.20 (s, 9H); LC/MS on 4.6×50 mm C-18 column, tᵣ=2.91 minutes (10 to 90% acetonitrile/water over 5 minutes at 4 ml/min with detection 220 nm, at 30° C.); ES-MS m/z 684 (M+H).

### Example 101

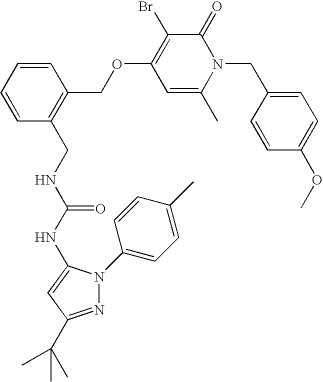

### 1-[2-({[3-bromo-1-(4-methoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea

### Step 1: Preparation of 0.2 M solution of 4-nitrophenyl [3-tert-butyl-1-(4-methylphenyl)-1H-pyrazol-5-yl]carbamate in methylene chloride

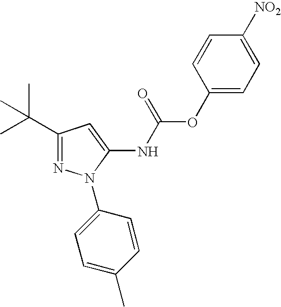

A solution of 459 mg of 3-tert-butyl-1-(4-methylphenyl)-1H-pyrazol-5-amine (2 mmol) was treated as described in part 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea to obtain a 0.2 M solution of 4-nitrophenyl [3-tert-butyl-1-(4-methylphenyl)-1H-pyrazol-5-yl]carbamate in methylene chloride. LC/MS on 4.6×50 mm C-18 column, tᵣ=3.32 minutes (10 to 90% acetonitrile/water over 5 minutes at 4 ml/min with detection 220 nm, at 30° C.); ES-MS m/z 395 (M+H).

### Step 2: Preparation of 1-[2-({[3-bromo-1-(4-methoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea

To a stirred solution of the 89 mg amine from step 4 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea above (0.2 mmol) and 42 uL of triethylamine (0.3 mmol) in 2 mL of methylene chloride was added 1.0 mL of the 0.2 M solution of 4-nitrophenyl [3-tert-butyl-1-phenyl-1H-pyrazol-5-yl]carbamate described in step 1 above (0.2 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 28 mg of 1-[2-({[3-bromo-1-(4-methoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea as a white solid (34%): ¹H NMR (400 MHz, DMSO-d₆) δ 8.17 (s, 1H), 7.44-7.48 (m, 1H), 7.22-7.33 (m, 7H), 7.04 (d, J=8.4 Hz, 2H), 6.96 (t, J=5.8 Hz, 1H), 6.86 (d, J=8.8 Hz, 2H), 6.46 (s, 1H), 6.21 (s, 1H), 5.30 (s, 2H), 5.21 (s, 2H), 4.31 (d, J=5.6 Hz, 2H), 3.67 (s, 3H), 2.31 (s, 3H), 2.26 (s, 3H), 1.20 (s, 9H); LC/MS on 4.6×50 mm C-18 column, tᵣ=3.19 minutes (10 to 90% acetonitrile/water over 5 minutes at 4 ml/min with detection 220 nm, at 30° C.); ES-MS m/z 714 (M+H).

### Example 102

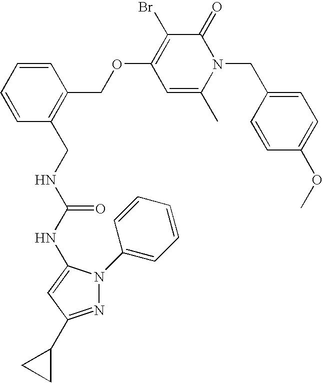

### 1-[2-({[3-bromo-1-(4-methoxybenzyl)-6-methyl-2-oxo-1,2-dihydropyridin-4-yl]oxy}methyl)benzyl]-3-(3-cyclopropyl-1-phenyl-1H-pyrazol-5-yl)urea

### Step 1: Preparation of 0.2 M solution of 4-nitrophenyl (3-cyclopropyl-1-phenyl-1H-pyrazol-5-yl)carbamate in methylene chloride

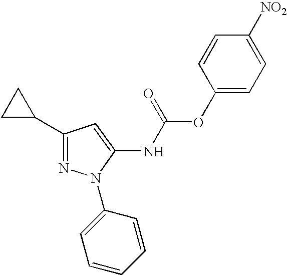

A solution of 399 mg of 3-amino-5-cyclopropyl-2-phenylpyrazole (2 mmol) was treated as described in part 5 of 1-[2-({[3-bromo-1-(4-methoxybenzyl)-6-methyl-2-oxo-1,2-dihydropyridin-4-yl]oxy}-methyl)benzyl]-3-[3-tert-butyl-1-(3-fluorophenyl)-1H-pyrazol-5-yl]urea to obtain a 0.2 M solution of 4-nitrophenyl (3-cyclopropyl-1-phenyl-1H-pyrazol-5-yl)carbamate in methylene chloride. LC/MS on 4.6×50 mm C-18 column, tᵣ=2.75 minutes (10 to 90% acetonitrile/water over 5 minutes at 4 ml/min with detection 220 nm, at 30° C.); ES-MS m/z 365 (M+H).

### Step 2: Preparation of 1-[2-({[3-bromo-1-(4-methoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea

To a stirred solution of the 89 mg amine from step 4 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea above (0.2 mmol) and 42 uL of triethylamine (0.3 mmol) in 2 mL of methylene chloride was added 1.0 mL of the 0.2 M solution of 4-nitrophenyl [3-tert-butyl-1-phenyl-1H-pyrazol-5-yl]carbamate described in step 1 above (0.2 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 28 mg of 1-[2-({[3-bromo-1-(4-methoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea as a white solid (34%): ¹H NMR (400 MHz, DMSO-d₆) δ 8.24 (s, 1H), 7.40-7.47 (m, 5H), 7.22-7.35 (m, 4H), 7.07 (d, J=8.8 Hz, 2H), 6.94 (t, J=5.8 Hz, 1H), 6.86 (d, J=8.4 Hz, 2H), 6.46 (s, 1H), 6.03 (s, 1H), 5.29 (s, 2H), 5.21 (s, 2H), 4.31 (d, J=6 Hz, 2H), 3.68 (s, 3H), 2.26 (s, 3H), 1.77-1.85 (m, 1H), 0.78-0.86 (m, 2H), 0.59-0.65 (m, 2H); LC/MS on 4.6×50 mm C-18 column, tᵣ=3.19 minutes (10 to 90% acetonitrile/water over 5 minutes at 4 ml/min with detection 220 nm, at 30° C.); ES-MS m/z 714 (M+H).

### Example 103

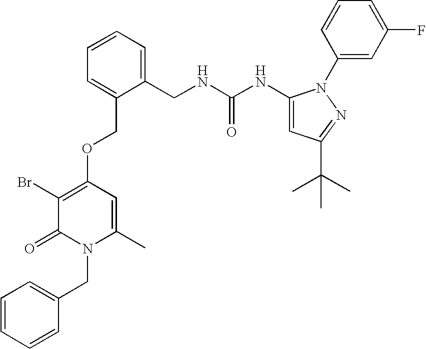

### 1-(2-{[(1-benzyl-3-bromo-6-methyl-2-oxo-1,2-dihydropyridin-4-yl)oxy]methyl}benzyl)-3-[3-tert-butyl-1-(3-fluorophenyl)-1H-pyrazol-5-yl]urea

### Step 1: Preparation of 1-benzyl-4-hydroxy-6-methylpyridin-2(1H)-one

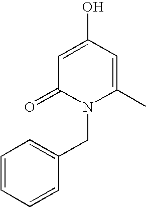

To a solution of 1.89 g of 4-hydroxy-6-methyl-2-pyrone (15 mmol) in 4 mL water in a 20 mL vial was dropwise 1.1 mL (10.05 mmol) of benzylamine. The vial was capped and placed in a heating block at 90 degrees Celsius for 15 hours, then cooled to room temperature. A brown oil had separated from the liquid, and the liquid was decanted off the oil. The brown oil was triturated with ethyl acetate and dried in vacuo overnight yielding 0.68 g of desired pyridinone as a yellow solid: ¹H NMR (400 MHz, d₆-DMSO) δ 2.13 (s, 3H), 5.15 (s, 2H), 5.56 (d, J=2.4 Hz, 1H), 5.76 (d, J=2.4 Hz, 1H), 7.06 (d, J=7.2 Hz, 2H), 7.20 (t, J=7.4 Hz, 1H), 7.29 (t, J=7.4 Hz, 2H), 10.4 (br s, 1H); C/MS on 4.6×50 mm C-18 column, tᵣ=1.59 minutes (10 to 90% acetonitrile/water over 5 minutes at 4 ml/min with detection 220 nm, at 30° C.); ES-MS m/z 216 (M+H).

### Step 2: Preparation of 2-{[(1-benzyl-6-methyl-2-oxo-1,2-dihydropyridin-4-yl)oxy]methyl}benzonitrile

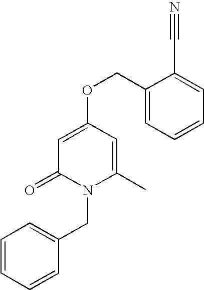

A mixture of 1.34 g of 1-benzyl-4-hydroxy-6-methylpyridin-2(1H)-one (6.2 mmol) from step 1, potassium carbonate (1.29 g, 9.32 mmol) and 1.34 g of alpha-bromotolunitrile (6.83 mmol) in dimethylformamide (11 mL) was stirred and heated to 65 degrees Celsius for 3 hours. The mixture was then cooled to room temperature, and the reaction mixture was slowly poured into 100 mL of ice water. No precipitation was observed, so the aqueous layer was extracted with ethyl acetate (100 mL). The organic layer was washed with 10% potassium carbonate (2×50 mL) and brine (1×50 mL), dried over magnesium sulfate and concentrated in vacuo to yield 1.59 g of product as a tan solid: LC/MS on 4.6×50 mm C-18 column, tᵣ=2.55 minutes (10 to 90% acetonitrile/water over 5 minutes at 4 ml/min with detection 220 nm, at 30° C.); ES-MS ml/331 (M+H).

### Step 3: Preparation of 2-{[(1-benzyl-3-bromo-6-methyl-2-oxo-1,2-dihydropyridin-4-yl)oxy]methyl}benzonitrile

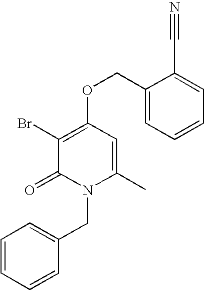

To a stirred solution of 1.59 g of 2-{[(1-benzyl-6-methyl-2-oxo-1,2-dihydropyridin-4-yl)oxy]methyl}benzonitrile from step 2 above (4.82 mmol) in 10 mL of anhydrous acetonitrile at 0 degrees Celsius was added 0.90 g of N-bromosuccinimide (5.05 mmol) in one portion. The mixture was stirred with cooling for one hour then the ice bath was removed. The mixture was treated with 5 mL of 10% sodium sulfite for one hour, and then the reaction mixture was partitioned between 100 mL ethyl acetate and 100 mL water. The organic phase was separated, washed with 10% potassium carbonate (2×100 mL) and 100 mL brine, dried over magnesium sulfate and concentrated in vacuo to give 2.2 g of crude product as a yellow solid. Recrystallization from isopropanol yielded 1.01 g of 2-{[(1-benzyl-3-bromo-6-methyl-2-oxo-1,2-dihydropyridin-4-yl)oxy]methyl}benzonitrile as a pale yellow solid (51%): ¹HNMR (400 MHz, d₃-CH₃Cl) 62.31 (s, 3H), 5.37 (s, 4H), 6.01 (s, 1H), 7.16 (d, J=6.8 Hz, 2H), 7.22-7.33 (m, 3H), 7.45 (t, J=7.2 Hz, 1H), 7.67-7.72 (m, 2H), 7.86 (d, J=7.6 Hz, 1H); LC/MS on 4.6×50 mm C-18 column, tᵣ=2.66 minutes (10 to 90% acetonitrile/water over 5 minutes at 4 ml/min with detection 220 nm, at 30° C.); ES-MS m/z 409 (M+H).

### Step 4: Preparation of 4-{[2-(aminomethyl)benzyl]oxy}-1-benzyl-3-bromo-6-methylpyridin-2(1H)-one

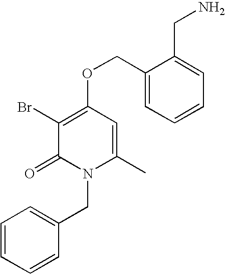

To a stirred solution of 0.877 g of 2-{[(1-benzyl-3-bromo-6-methyl-2-oxo-1,2-dihydropyridin-4-yl)oxy]methyl}benzonitrile from step 3 above (2.14 mmol) in 5 anhydrous tetrahydrofuran at 0 degrees Celsius was added 4.3 mL of 1.0 M solution of borane-tetrahydrofuran complex in tetrahydrofuran (4.3 mmol) dropwise over 10 minutes. The addition was accompanied by vigorous off-gassing. After complete addition, the reaction mixture was warmed to room temperature and stirred for 15 hours. An additional 4.3 mL of 1.0 M solution of borane-tetrahydrofuran complex in tetrahydrofuran (4.3 mmol) dropwise, and the reaction was stirred for an additional 4 hours. Then the flask was immersed in an ice bath, and 2 mL of methanol was carefully added dropwise to the mixture. The addition was again accompanied by vigorous off-gassing. The mixture was warmed to room temperature and volatiles were removed in vacuo leaving 1 g of crude product. The crude product was dissolved in a mixture of 30 mL methanol and 30 mL methylene chloride in a 150 mL wide mouth jar, and 15 g of polymer-bound sulfonic acid (60 mmol) was added. The jar was capped, and the mixture was agitated on a tabletop shaker for 1 hour. The resin was filtered and washed successively with methanol (3×50 mL) and methylene chloride (3×50 mL). The resin was transferred back to the jar and 30 mL of methylene chloride and 30 mL of 7 N ammonia in methanol was added. The mixture was agitated for 1 hour on the tabletop shaker, then the resin was filtered and washed with successively with methanol (2×50 mL) and methylene chloride (2×50 mL). The resin was retreated with 30 mL of methylene chloride and 30 mL of 7 N ammonia in methanol for 2 hour on the tabletop shaker, and the resin was again filtered and washed with successively with methanol (2×50 mL) and methylene chloride (2×50 mL). All filtrates were combined and concentrated in vacuo to yield 1.25 g of product as a tan solid; ¹H NMR (400 MHz, d₃-CH₃Cl) δ 2.27 (s, 3H), 3.95 (s, 2H), 5.29 (s, 2H), 5.35 (s, 2H), 6.09 (s, 1H), 7.14-7.18 (m, 3H), 7.24-7.46 (m, 6H); LC/MS on 4.6×50 mm C-18 column, tᵣ=1.49 minutes (10 to 90% acetonitrile/water over 5 minutes at 4 ml/min with detection 220 nm, at 30° C.); ES-MS m/z 413 (M+H).

### Step 5: Preparation of 1-(2-{[(1-benzyl-3-bromo-6-methyl-2-oxo-1,2-dihydropyridin-4-yl)oxy]methyl}benzyl)-3-[(3-tert-butyl-1-(3-fluorophenyl)-1H-pyrazol-5-yl]urea

To a stirred solution of the 50 mg 4-{[2-(aminomethyl)benzyl]oxy}-1-benzyl-3-bromo-6-methylpyridin-2(1H)-one from step 4 above (0.12 mmol) and 24 uL of triethylamine (0.17 mmol) in 1 mL of methylene chloride was added 0.60 mL of the 0.2 M solution of 4-nitrophenyl [3-tert-butyl-1-(3-fluorophenyl)-1H-pyrazol-5-yl]carbamate described in step 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea (0.12 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 25 mg of 1-(2-{[(1-benzyl-3-bromo-6-methyl-2-oxo-1,2-dihydropyridin-4-yl)oxy]methyl}benzyl)-3-[3-tert-butyl-1-(3-fluorophenyl)-1H-pyrazol-5-yl]urea as a white solid (30%): ¹H NMR (400 MHz, DMSO-d₆) δ 8.37 (s, 1H), 7.43-7.49 (m, 2H), 7.2-7.35 (m, 8H), 7.13-7.18 (m, 1H), 7.00-7. (m, 3H), 6.50 (s, 1H), 6.24 (s, 1H), 5.31 (s, 2H), 5.29 (s, 2H), 4.32 (d, J=5.6 Hz, 2H), 2.24 (s, 3H), 1.21 (s, 9H); LC/MS on 4.6×50 mm C-18 column, tᵣ=3.28 minutes (10 to 90% acetonitrile/water over 5 minutes at 4 ml/min with detection 220 nm, at 30° C.); ES-MS m/z 672 (M+H).

### Example 104

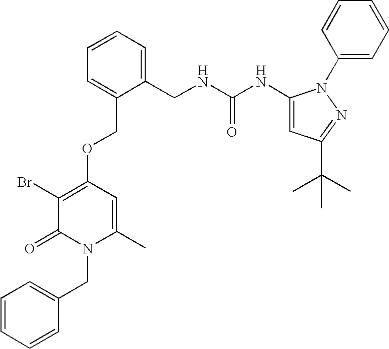

### 1-(2-{[(1-benzyl-3-bromo-6-methyl-2-oxo-1,2-dihydropyridin-4-yl)oxy]methyl}benzyl)-3-(3-tert-butyl-1-phenyl-1H-pyrazol-5-yl)urea

### Step 1: Preparation of 0.2 M solution of 4-nitrophenyl [3-tert-butyl-1-phenyl-1H-pyrazol-5-yl]carbamate in methylene chloride

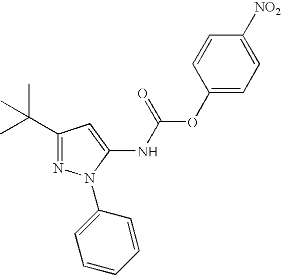

To a solution of 431 mg of 3-tert-butyl-1phenyl)-1H-pyrazol-5-amine (2 mmol) was treated as described in part 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea to obtain a 0.2 M solution of 4-nitrophenyl [3-tert-butyl-1-phenyl-1H-pyrazol-5-yl]carbamate in methylene chloride. LC/MS on 4.6×50 mm C-18 column, tᵣ=3.16 minutes (10 to 90% acetonitrile/water over 5 minutes at 4 ml/min with detection 220 nm, at 30° C.); ES-MS m/z 381 (M+H).

### Step 2: Preparation of 1-(2-{[(1-benzyl-3-bromo-6-methyl-2-oxo-1,2-dihydropyridin-4-yl)oxy]methyl}benzyl)-3-(3-tert-butyl-1-phenyl-1H-pyrazol-5-yl)urea

To a stirred solution of the 50 mg amine from step 4 in 1-(2-{[(1-benzyl-3-bromo-6-methyl-2-oxo-1,2-dihydropyridin-4-yl)oxy]methyl}benzyl)-3-[3-tert-butyl-1-(3-fluorophenyl)-1H-pyrazol-5-yl]urea above (0.12 mmol) and 24 uL of triethylamine (0.17 mmol) in 1 mL of methylene chloride was added 0.60 mL of the 0.2 M solution of 4-nitrophenyl [3-tert-butyl-1-(4-methylphenyl)-1H-pyrazol-5-yl]carbamate described in step 1 above (0.12 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 15 mg of 1-(2-{[(1-benzyl-3-bromo-6-methyl-2-oxo-1,2-dihydropyridin-4-yl)oxy]methyl}benzyl)-3-(3-tert-butyl-1-phenyl-1H-pyrazol-5-yl)urea as a white solid (19%): ¹H NMR (400 MHz, DMSO-d₆) δ 8.28 (s, 1H), 7.41-7.48 (m, 5H), 7.21-7.35 (m, 7H), 7.07 (d, J=7.6 Hz, 2H), 7.01 (t, J=5.8 Hz, 1H), 6.50 (s, 1H), 6.23 (s, 1H), 5.32 (s, 2H), 5.29 (s, 2H), 4.32 (d, J=6 Hz, 2H), 2.24 (s, 3H), 1.21 (s, 9H); LC/MS on 4.6×50 mm C-18 column, tᵣ=3.16 minutes (10 to 90% acetonitrile/water over 5 minutes at 4 ml/min with detection 220 nm, at 30° C.); ES-MS m/z 654 (M+H).

### Example 105

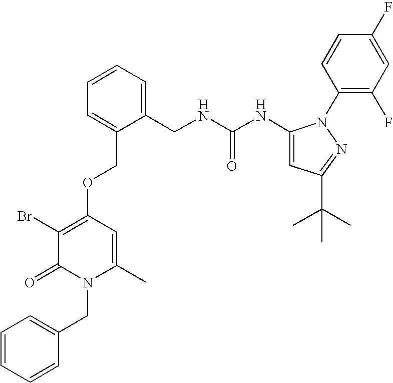

### 1-(2-{[(1-benzyl-3-bromo-6-methyl-2-oxo-1,2-dihydropyridin-4-yl)oxy]methyl}benzyl)-3-[3-tert-butyl-1-(2,4-difluorophenyl)-1H-pyrazol-5-yl]urea

### Step 1: Preparation of 0.2 M solution of 4-nitrophenyl [3-tert-butyl-1-(2,4-difluorophenyl)-1H-pyrazol-5-yl]carbamate in methylene chloride

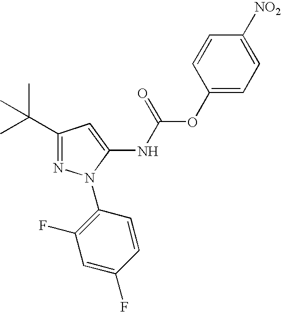

To a solution of 503 mg of 3-tert-butyl-1-(2,4-difluorophenyl)-1H-pyrazol-5-amine (2 mmol)) was treated as described in part 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]ureato obtain a 0.2 M solution of [3-tert-butyl-1-(2,4-difluorophenyl)-1H-pyrazol-5-yl]carbamate in methylene chloride. LC/MS on 4.6×50 mm C-18 column, tᵣ=3.16 minutes (10 to 90% acetonitrile/water over 5 minutes at 4 ml/min with detection 220 nm, at 30° C.); ES-MS m/z 417 (M+H).

### Step 2: Preparation of 1-(2-{[(1-benzyl-3-bromo-6-methyl-2-oxo-1,2-dihydropyridin-4-yl)oxy]methyl}benzyl)-3-[3-tert-butyl-1-(2,4-difluorophenyl)-1H-pyrazol-5-yl]urea

To a stirred solution of the 50 mg amine from step 4 in 1-(2-{[(1-benzyl-3-bromo-6-methyl-2-oxo-1,2-dihydropyridin-4-yl)oxy]methyl}benzyl)-3-[3-tert-butyl-1-(3-fluorophenyl)-1H-pyrazol-5-yl]urea above (0.12 mmol) and 24 uL of triethylamine (0.17 mmol) in 1 mL of methylene chloride was added 0.60 mL of the 0.2 M solution of 4-nitrophenyl [3-tert-butyl-1-(2,4-difluorophenyl)-1H-pyrazol-5-yl]carbamate described in step 1 above (0.12 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 28 mg of 1-(2-{[(1-benzyl-3-bromo-6-methyl-2-oxo-1,2-dihydropyridin-4-yl)oxy]methyl}benzyl)-3-[3-tert-butyl-1-(2,4-difluorophenyl)-1H-pyrazol-5-yl]urea as a white solid (34%): ¹H NMR (400 MHz, DMSO-d₆) δ 8.28 (s, 1H), 7.45-7.55 (m, 3H), 7.17-7.32 (m, 7H), 7.07 (d, J=7.6 Hz, 2H), 6.86 (m, 1H), 6.50 (s, 1H), 6.22 (s, 1H), 5.31 (s, 2H), 5.29 (s, 2H), 4.32 (d, J=4.8 Hz, 2H), 2.25 (s, 3H), 1.19 (s, 9H); LC/MS on 4.6×50 mm C-18 column, tᵣ=3.17 minutes (10 to 90% acetonitrile/water over 5 minutes at 4 ml/min with detection 220 nm, at 30° C.); ES-MS m/z 690 (M+H).

### Example 106

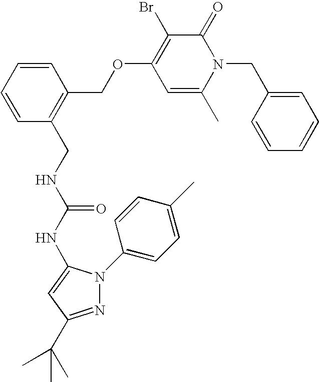

### 1-(2-{[(1-benzyl-3-bromo-6-methyl-2-oxo-1,2-dihydropyridin-4-yl)oxy]methyl}benzyl)-3-[3-tert-butyl-1-(4-methylphenyl)-1H-pyrazol-5-yl]urea

To a stirred solution of the 83 mg amine from step 4 of the preparation of 1-(2-{[(1-benzyl-3-bromo-6-methyl-2-oxo-1,2-dihydropyridin-4-yl)oxy]methyl}benzyl)-3-[3-tert-butyl-1-(3-fluorophenyl)-1H-pyrazol-5-yl]urea above (0.2 mmol) and 42 uL of triethylamine (0.3 mmol) in 2 mL of methylene chloride was added 1 mL of the 0.2 M solution of 4-nitrophenyl [3-tert-butyl-1-(4-methylphenyl)-1H-pyrazol-5-yl]carbamate described in step 1 of 1-[2-({[3-bromo-1-(4-methoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea (0.2 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 65 mg of 1-(2-{[(1-benzyl-3-bromo-6-methyl-2-oxo-1,2-dihydropyridin-4-yl)oxy]methyl}benzyl)-3-[3-tert-butyl-1-(4-methylphenyl)-1H-pyrazol-5-yl]urea as a white solid (49%): ¹H NMR (400 MHz, DMSO-d₆) δ 8.17 (s, 1H), 7.45-7.48 (m, 1H), 7.22-7.34 (m, 10H), 7.07 (d, J=7.2 Hz, 2H), 6.96 (t, J=5.8 Hz, 1H), 6.49 (s, 1H), 6.21 (s, 1H), 5.31 (s, 2H), 5.29 (s, 2H), 4.32 (d, J=5.6 Hz, 2H), 2.31 (s, 3H), 2.24 (s, 3H), 1.20 (s, 9H); LC/MS on 4.6×50 mm C-18 column, tᵣ=3.27 minutes (10 to 90% acetonitrile/water over 5 minutes at 4 ml/min with detection 220 nm, at 30° C.); ES-MS m/z 668 (M+H).

### Example 107

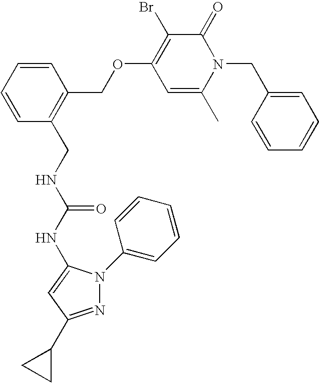

### 1-(2-{[(1-benzyl-3-bromo-6-methyl-2-oxo-1,2-dihydropyridin-4-yl)oxy]methyl}benzyl)-3-(3-cyclopropyl-1-phenyl-1H-pyrazol-5-yl)urea

To a stirred solution of the 83 mg amine from step 4 of the preparation of 1-(2-{[(1-benzyl-3-bromo-6-methyl-2-oxo-1,2-dihydropyridin-4-yl)oxy]methyl}benzyl)-3-[3-tert-butyl-1-(3-fluorophenyl)-1H-pyrazol-5-yl]urea above (0.2 mmol) and 42 uL of triethylamine (0.3 mmol) in 2 mL of methylene chloride was added 1 mL of the 0.2 M solution of 4-nitrophenyl (3-cyclopropyl-1-phenyl-1H-pyrazol-5-yl)carbamate described in step 1 of 1-[2-({[3-bromo-1-(4-methoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea (0.2 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 65 mg of 1-(2-{[(1-benzyl-3-bromo-6-methyl-2-oxo-1,2-dihydropyridin-4-yl)oxy]methyl}benzyl)-3-[3-tert-butyl-1-(4-methylphenyl)-1H-pyrazol-5-yl]urea as a white solid (51%): ¹H NMR (400 MHz, DMSO-d₆) δ 8.24 (s, 1H), 7.40-7.48 (m, 5H), 7.21-7.35 (m, 7H), 7.07 (d, J=7.2 Hz, 2H), 6.95 (t, J=5.8 Hz, 1H), 6.49 (s, 1H), 6.03 (s, 1H), 5.30 (s, 2H), 5.29 (s, 2H), 4.31 (d, J=5.6 Hz, 2H), 2.25 (s, 3H), 1.77-1.85 (m, 1H), 0.78-0.86 (m, 2H), 0.59-0.67 (m, 2H); LC/MS on 4.6×50 mm C-18 column, tᵣ=3.27 minutes (10 to 90% acetonitrile/water over 5 minutes at 4 ml/min with detection 220 nm, at 30° C.); ES-MS m/z 668 (M+H).

### Example 108

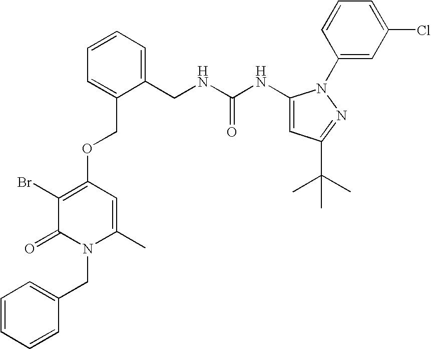

### 1-(2-{[(1-benzyl-3-bromo-6-methyl-2-oxo-1,2-dihydropyridin-4-yl)oxy]methyl}benzyl)-3-[3-tert-butyl-1-(3-chlorophenyl)-1H-pyrazol-5-yl]urea

To a stirred solution of the 50 mg amine from step 4 of the preparation of 1-(2-{[(1-benzyl-3-bromo-6-methyl-2-oxo-1,2-dihydropyridin-4-yl)oxy]methyl}benzyl)-3-[3-tert-butyl-1-(3-fluorophenyl)-1H-pyrazol-5-yl]urea above (0.12 mmol) and 24 uL of triethylamine (0.17 mmol) in 2 mL of methylene chloride was added 0.6 mL of the 0.2 M solution of 4-nitrophenyl [3-tert-butyl-1-(3-chlorophenyl)-1H-pyrazol-5-yl]carbamate in methylene chloride described in step 1 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-chlorophenyl)-1H-pyrazol-5-yl]urea (0.12 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 35 mg of 1-(2-{[(1-benzyl-3-bromo-6-methyl-2-oxo-1,2-dihydropyridin-4-yl)oxy]methyl}benzyl)-3-[3-tert-butyl-1-(3-chlorophenyl)-1H-pyrazol-5-yl]urea as a white solid (42%): ¹H NMR (400 MHz, DMSO-d₆) δ 8.40 (s, 1H), 7.54 (s, 1H), 7.42-7.47 (m, 3H), 7.36-7.40 (m, 1H), 7.21-7.33 (m, 6H), 7.00-7.08 (m, 3H), 6.50 (s, 1H), 6.23 (s, 1H), 5.31 (s, 2H), 5.29 (s, 2H), 4.32 (d, J=5.6 Hz, 2H), 2.24 (s, 3H), 1.21 (s, 9H); LC/MS on 4.6×50 mm C-18 column, tᵣ=3.42 minutes (10 to 90% acetonitrile/water over 5 minutes at 4 ml/min with detection 220 nm, at 30° C.); ES-MS m/z 688 (M+H).

### Example 109

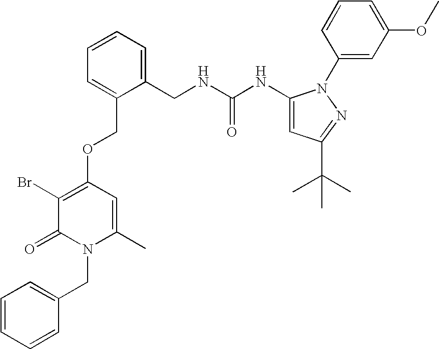

### 1-(2-{[(1-benzyl-3-bromo-6-methyl-2-oxo-1,2-dihydropyridin-4-yl)oxy]methyl}benzyl)-3-[3-tert-butyl-1-(3-methoxyphenyl)-1H-pyrazol-5-yl]urea

To a stirred solution of the 50 mg amine from step 4 of the preparation of 1-(2-{[(1-benzyl-3-bromo-6-methyl-2-oxo-1,2-dihydropyridin-4-yl)oxy]methyl}benzyl)-3-[3-tert-butyl-1-(3-fluorophenyl)-1H-pyrazol-5-yl]urea above (0.12 mmol) and 24 uL of triethylamine (0.17 mmol) in 2 mL of methylene chloride was added 0.6 mL of the 0.2 M solution of 4-nitrophenyl [3-tert-butyl-1-(3-methoxyphenyl)-1H-pyrazol-5-yl]carbamate in methylene chloride described in step 1 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-methoxyphenyl)-1H-pyrazol-5-yl]urea (0.12 mmol). The mixture was stirred at room temperature for 15 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 23 mg of 1-(2-{[(1-benzyl-3-bromo-6-methyl-2-oxo-1,2-dihydropyridin-4-yl)oxy]methyl}benzyl)-3-[3-tert-butyl-1-(3-methoxyphenyl)-1H-pyrazol-5-yl]urea as a white solid (27%): ¹H NMR (400 MHz, DMSO-d₆) δ 8.24 (s, 1H), 7.45-7.48 (m, 1H), 7.21-7.36 (m, 7H), 7.07 (d, J=7.2 Hz, 2H), 6.99-7.03 (m, 3H), 6.90-6.92 (m, 1H), 6.49 (s, 1H), 6.23 (s, 1H), 5.31 (s, 2H), 5.29 (s, 2H), 4.33 (d, J=5.6 Hz, 2H), 3.73 (s, 3H), 2.24 (s, 3H), 1.21 (s, 9H); LC/MS on 4.6×50 mm C-18 column, tᵣ=3.20 minutes (10 to 90% acetonitrile/water over 5 minutes at 4 ml/min with detection 220 nm, at 30° C.); ES-MS m/z 684 (M+H).

### Example 110

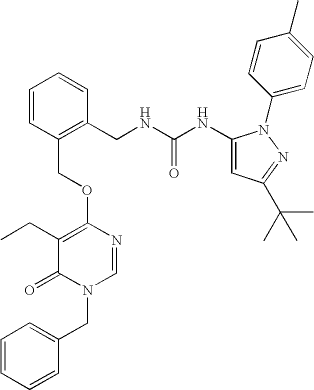

### 1-(2-{[(1-benzyl-5-ethyl-6-oxo-1,6-dihydropyrimidin-4-yl)oxy]methyl}benzyl)-3-[3-tert-butyl-1-(4-methylphenyl)-1H-pyrazol-5-yl]urea

### Step 1: Preparation of 2-{[(5-ethyl-6-oxo-1,6-dihydropyrimidin-4-yl)oxy]methyl}benzonitrile

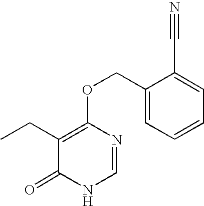

To a suspension of 1.36 g of 60% sodium hydride oil dispersion (33.9 mmol) in 70 mL dimethylformamide at 0 degrees Celsius was added portionwise 5 g of 4,6-dihydroxy-5-ethylpyrimidine (35.7 mmol) over 10 minutes. Addition resulted in vigorous off-gassing. The mixture was warmed to room temperature and stirred for 3 hours. After cooling back down to 0 degrees Celsius, a solution of 7.0 g of alpha-bromotolunitrile (35.7 mmol) in 5 mL dimethylformamide was added dropwise over 15 minutes. After complete addition, the mixture was warmed to room temperature and stirred for 2.5 hours. The crude reaction mixture was slowly added to 500 mL of vigorously stirred ice water, resulting in a tan precipitate. The solid was collected by vacuum filtrates and washed three times with 200 mL water and three times with 200 mL of diethyl ether. The solid was dried in vacuo and triturated with diethyl ether to yield 2.66 g of 2-{[(5-ethyl-6-oxo-1,6-dihydropyrimidin-4-yl)oxy]methyl}benzonitrile as an off-white solid (29%): ¹H NMR (400 MHz, DMSO-d₆) δ 0.94 (t, J=7.2 Hz, 3H), 2.33 (q, J=7.2 Hz, 2H), 5.47 (s, 214), 7.52 (t, J=7.2 Hz, 1H), 7.62 (d, J=7.6 Hz, 1H), 7.71 (t, J=7.4 Hz, 1H), 7.87 (d, J=7.6 Hz, 1H), 8.01 (s, 1H), 12.4 (br s, 1H); LC/MS on 4.6×50 mm C-18 column, tᵣ=1.88 minutes (10 to 90% acetonitrile/water over 5 minutes at 4 ml/min with detection 220 nm, at 30° C.); ES-MS m/z 256 (M+H).

### Step 2: Preparation of 2-{[(1-benzyl-5-ethyl-6-oxo-1,6-dihydropyrimidin-4-yl)oxy]methyl}benzonitrile

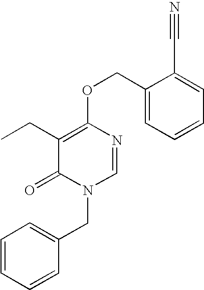

To a suspension of 80 mg of 60% sodium hydride oil dispersion (2 mmol) in 70 mL dimethylformamide at 0 degrees Celsius was added portionwise 434 mg of 2-{[(5-ethyl-6-oxo-1,6-dihydropyrimidin-4-yl)oxy]methyl}benzonitrile (1.7 mmol) over 10 minutes. Addition resulted in vigorous off-gassing. The mixture was warmed to room temperature and stirred for 2 hours. After cooling back down to 0 degrees Celsius, a solution of 342 mg of benzyl bromide (1.7 mmol) in 1.2 mL dimethylformamide was added dropwise over 10 minutes. After complete addition, the mixture was warmed to room temperature and stirred for 2.5 hours. The crude reaction mixture was slowly added to 50 mL of vigorously stirred ice water, resulting in a tan precipitate. The solid was collected by vacuum filtrates and washed three times with 20 mL water and three times with 20 mL of diethyl ether. The solid was dried in vacuo and triturated with ethyl acetate/hexane to yield 438 mg of 2-{[(1-benzyl-5-ethyl-6-oxo-1,6-dihydropyrimidin-4-yl)oxy]methyl}benzonitrile as an off-white solid (75%): ¹HNMR (400 MHz, d₃-CH₃Cl) δ 1.10 (t, J=7.4 Hz, 3H), 2.55 (q, J=7.5 Hz, 2H), 5.07 (s, 2H), 5.54 (s, 2H), 7.28-7.37 (m, 5H), 7.40 (td, J=7.4, 1.7 Hz, 1H), 7.54-7.61 (m, 2H), 7.67 (d, J=7.6 Hz, 1H), 7.89 (s, 1H); LC/MS on 4.6×50 mm C-18 column, tᵣ=2.85 minutes (10 to 90% acetonitrile/water over 5 minutes at 4 ml/min with detection 220 nm, at 30° C.); ES-MS m/z 346 (M+H).

### Step 3: Preparation of 6-{[2-(aminomethyl)benzyl]oxy}-3-benzyl-5-ethylpyrimidin-4(3H)-one

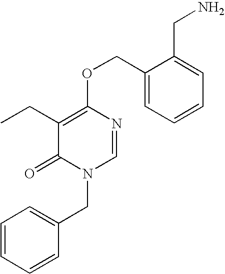

To a mixture of 229 mg of 2-{[(1-benzyl-5-ethyl-6-oxo-1,6-dihydropyrimidin-4-yl)oxy]methyl}benzonitrile (0.66 mmol) and 315 mg of cobalt chloride hexahydrate (1.32 mmol) in 5 mL of anhydrous methanol at 0 degrees Celsius was added portionwise 250 mg of sodium borohydride (6.6 mmol) over 30 minutes. Addition resulted in vigorous off-gassing and color change to black. The mixture was warmed to room temperature and stirred for 15 minutes, then 5 mL of 5% aqueous hydrogen chloride solution and 5 mL of water was added. The solution was basified by the addition of solid sodium carbonate to pH>9. The mixture was extracted twice with 50 mL methylene chloride, then the combined organic layers were dried over magnesium sulfate and concentrated in vacuo. The crude was dissolved in a mixture of 5 mL of methanol and 5 mL methylene chloride, and 1.3 g of polymer-bound sulfonic acid (Argonaut) was added to the jar was capped, and the mixture was agitated on a tabletop shaker for 1 hour. The resin was filtered and washed successively with methanol (3×50 mL) and methylene chloride (3×50 mL). The resin was treated with 5 mL of methylene chloride and 5 mL of 7 N ammonia in methanol. The mixture was agitated for 30 minutes on the tabletop shaker, then the resin was filtered and washed with successively with methanol (2×5 mL) and methylene chloride (2×5 mL). The resin was retreated with 5 mL of methylene chloride and 5 mL of 7 N ammonia in methanol for 30 minutes on the tabletop shaker, and the resin was again filtered and washed with successively with methanol (2×5 mL) and methylene chloride (2×5 mL). All filtrates were combined and concentrated in vacuo to yield 142 mg of product as a tan solid (61%); LC/MS on 4.6×50 mm C-18 column, tᵣ=1.75 minutes (10 to 90% acetonitrile/water over 5 minutes at 4 ml/min with detection 220 nm, at 30° C.); ES-MS m/z 350 (M+H).

### Step 4: Preparation of 1-(2-{[(1-benzyl-5-ethyl-6-oxo-1,6-dihydropyrimidin-4-yl)oxy]methyl}benzyl)-3-[3-tert-butyl-1-(4-methylphenyl)-1H-pyrazol-5-yl]urea

To a stirred solution of the 35 mg 6-{[2-(aminomethyl)benzyl]oxy}-3-benzyl-5-ethylpyrimidin-4(3H)-one from step 3 above (0.1 mmol) and 28 uL of triethylamine (0.2 mmol) in 1 mL of methylene chloride was added 0.5 mL of the 0.2 M solution of 4-nitrophenyl [3-tert-butyl-1-(4-methylphenyl)-1H-pyrazol-5-yl]carbamate described in step 1 of 1-[2-({[3-bromo-1-(4-methoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea (0.1 mmol). The mixture was stirred at room temperature for 1 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 35 mg of 1-(2-{[(1-benzyl-5-ethyl-6-oxo-1,6-dihydropyrimidin-4-yl)oxy]methyl}benzyl)-3-[3-tert-butyl-1-(4-methylphenyl)-1H-pyrazol-5-yl]urea as a white solid (51%): ¹HNMR (400 MHz, DMSO-d₆) δ 8.47 (1H, s), 8.13 (1H, s), 7.19-7.29 (13H, m), 6.78-6.98 (1H, m), 6.18 (1H, s), 5.36 (2H, s), 5.03 (2H, s), 4.28 (2H, d, J=5.5 Hz), 2.26-2.33 (5H, m), 1.19 (9H, s), 0.89 (3H, t, J=7.3 Hz); LC/MS on 4.6×50 mm C-18 column, tᵣ=3.36 minutes (10 to 90% acetonitrile/water over 5 minutes at 4 ml/min with detection 220 nm, at 30° C.); ES-MS m/z 605 (M+H).

### Example 111

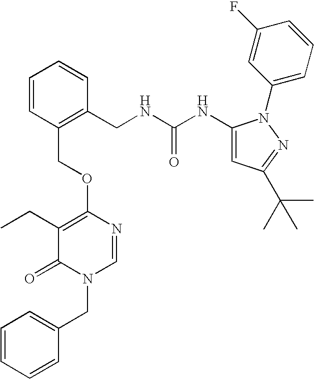

### 1-(2-{[(1-benzyl-5-ethyl-6-oxo-1,6-dihydropyrimidin-4-yl)oxy]methyl}benzyl)-3-[3-tert-butyl-1-(3-fluorophenyl)-1H-pyrazol-5-yl]urea

To a stirred solution of the 35 mg 6-{[2-(aminomethyl)benzyl]oxy}-3-benzyl-5-ethylpyrimidin-4(3H)-one from step 3 of the preparation of 1-(2-{[(1-benzyl-5-ethyl-6-oxo-1,6-dihydropyrimidin-4-yl)oxy]methyl}benzyl)-3-[3-tert-butyl-1-(4-methylphenyl)-1H-pyrazol-5-yl]urea above (0.1 mmol) and 28 uL of triethylamine (0.2 mmol) in 1 mL of methylene chloride was added 0.5 mL of the 0.2 M solution of 4-nitrophenyl [3-tert-butyl-1-(3-fluorophenyl)-1H-pyrazol-5-yl]carbamate described in step 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea (0.1 mmol). The mixture was stirred at room temperature for 1 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 33 mg of 1-(2-{[(1-benzyl-5-ethyl-6-oxo-1,6-dihydropyrimidin-4-yl)oxy]methyl}benzyl)-3-[3-tert-butyl-1-(3-fluorophenyl)-1H-pyrazol-5-yl]urea as a white solid (53%): ¹H NMR (400 MHz, DMSO-d₆) δ 8.46 (1H, s), 8.18 (1H, s), 7.41 (4H, s), 7.17-7.35 (10H, m), 6.88 (1H, s), 6.19 (1H, s), 5.35 (2H, s), 5.02 (2H, s), 4.27-4.29 (2H, m), 2.27-2.30 (2H, m), 1.19 (9H, s), 0.88 (3H, t, J=7.0 Hz); LC/MS on 4.6×50 mm C-18 column, tᵣ=3.27 minutes (10 to 90% acetonitrile/water over 5 minutes at 4 ml/min with detection 220 nm, at 30° C.); ES-MS m/z 591 (M+H).

### Example 112

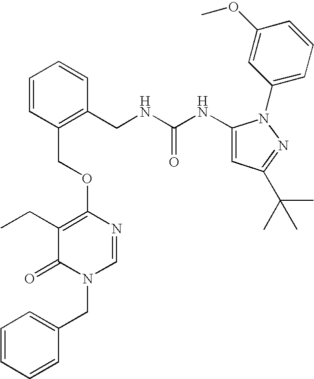

### 1-(2-{[(1-benzyl-5-ethyl-6-oxo-1,6-dihydropyrimidin-4-yl)oxy]methyl}benzyl)-3-[3-tert-butyl-1-(3-methoxyphenyl)-1H-pyrazol-5-yl]urea

To a stirred solution of the 35 mg 6-{[2-(aminomethyl)benzyl]oxy}-3-benzyl-5-ethylpyrimidin-4(3H)-one from step 3 of the preparation of 1-(2-{[(1-benzyl-5-ethyl-6-oxo-1,6-dihydropyrimidin-4-yl)oxy]methyl}benzyl)-3-[3-tert-butyl-1-(4-methylphenyl)-1H-pyrazol-5-yl]urea above (0.1 mmol) and 28 uL of triethylamine (0.2 mmol) in 1 mL of methylene chloride was added 0.5 mL of the 0.2 M solution of 0.2 M solution of 4-nitrophenyl [3-tert-butyl-1-(3-methoxyphenyl)-1H-pyrazol-5-yl]carbamate described in step 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-methoxyphenyl)-1H-pyrazol-5-yl]urea (0.1 mmol). The mixture was stirred at room temperature for 1 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 33 mg of 1-(2-{[(1-benzyl-5-ethyl-6-oxo-1,6-dihydropyrimidin-4-yl)oxy]methyl}benzyl)-3-[3-tert-butyl-1-(3-methoxyphenyl)-1H-pyrazol-5-yl]urea as a white solid (53%): ¹H NMR (400 MHz, DMSO-d₆) δ 8.43-8.51 (1H, m), 8.20 (1H, s), 7.31-7.35 (1H, m), 7.19-7.31 (8H, m), 6.97-7.01 (2H, m), 6.87-6.95 (2H, m), 6.20 (1H, s), 5.36 (2H, s), 5.03 (2H, s), 4.29 (2H, d, J=5.5 Hz), 3.71 (3H, s), 2.29 (3H, q, J=7.3 Hz), 1.20 (9H, s), 0.89 (3H, t, J=7.3 Hz); LC/MS on 4.6×50 mm C-18 column, tᵣ=3.37 minutes (10 to 90% acetonitrile/water over 5 minutes at 4 ml/min with detection 220 nm, at 30° C.); ES-MS m/z 609 (M+H).

### Example 113

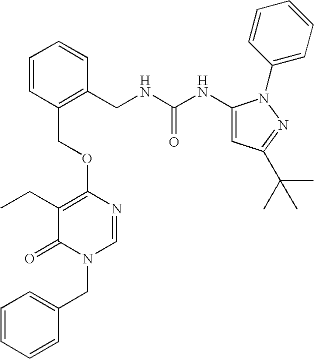

### 1-(2-{[(1-benzyl-5-ethyl-6-oxo-1,6-dihydropyrimidin-4-yl)oxy]methyl}benzyl)-3-(3-tert-butyl-1-phenyl-1H-pyrazol-5-yl)urea

To a stirred solution of the 35 mg 6-{[2-(aminomethyl)benzyl]oxy}-3-benzyl-5-ethylpyrimidin-4(3H)-one from step 3 of the preparation of 1-(2-{[(1-benzyl-5-ethyl-6-oxo-1,6-dihydropyrimidin-4-yl)oxy]methyl}benzyl)-3-[3-tert-butyl-1-(4-methylphenyl)-1H-pyrazol-5-yl]urea above (0.1 mmol) and 28 uL of triethylamine (0.2 mmol) in 1 mL of methylene chloride was added 0.5 mL of the 0.2 M solution of 4-nitrophenyl [3-tert-butyl-1-phenyl-1H-pyrazol-5-yl]carbamate in methylene chloride described in step 1 of 1-(2-{[(1-benzyl-3-bromo-6-methyl-2-oxo-1,2-dihydropyridin-4-yl)oxy]methyl}benzyl)-3-(3-tert-butyl-1-phenyl-1H-pyrazol-5-yl)urea (0.1 mmol). The mixture was stirred at room temperature for 1 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 40 mg of 1-(2-{[(1-benzyl-5-ethyl-6-oxo-1,6-dihydropyrimidin-4-yl)oxy]methyl}benzyl)-3-(3-tert-butyl-1-phenyl-1H-pyrazol-5-yl)urea as a white solid (67%): ¹H NMR (400 MHz, DMSO-d₆) δ 8.46 (1H, s), 8.18 (1H, s), 7.41 (4H, s), 7.17-7.35 (10H, m), 6.88 (1H, s), 6.19 (1H, s), 5.35 (2H, s), 5.02 (2H, s), 4.27-4.29 (2H, m), 2.27-2.30 (2H, m), 1.19 (9H, s), 0.88 (3H, t, J=7.0 Hz); LC/MS on 4.6×50 mm C-18 column, tᵣ=3.27 minutes (10 to 90% acetonitrile/water over 5 minutes at 4 ml/min with detection 220 nm, at 30° C.); ES-MS m/z 591(M+H).

### Example 114

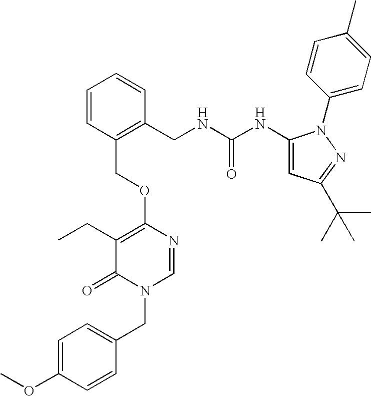

### 1-[3-tert-butyl-1-(4-methylphenyl)-1H-pyrazol-5-yl]-3-[2-({[5-ethyl-1-(4-methoxybenzyl)-6-oxo-1,6-dihydropyrimidin-4-yl]oxy}methyl)benzyl]urea

### Step 1: Preparation of 2-({[5-ethyl-1-(4-methoxybenzyl)-6-oxo-1,6-dihydropyrimidin-4-yl]oxy}methyl)benzonitrile

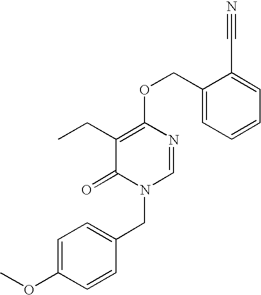

To a suspension of 80 mg of 60% sodium hydride oil dispersion (2 mmol) in 70 mL dimethylformamide at 0 degrees Celsius was added portionwise 434 mg of 2-{[(5-ethyl-6-oxo-1,6-dihydropyrimidin-4-yl)oxy]methyl}benzonitrile from step 1 of 1-(2-{[(1-benzyl-5-ethyl-6-oxo-1,6-dihydropyrimidin-4-yl)oxy]methyl}benzyl)-3-[3-tert-butyl-1-(4-methylphenyl)-1H-pyrazol-5-yl]urea (1.7 mmol) over 10 minutes. Addition resulted in vigorous off-gassing. The mixture was warmed to room temperature and stirred for 2 hours. After cooling back down to 0 degrees Celsius, a solution of 313 mg of 4-methoxybenzyl chloride (1.7 mmol) in 1.2 mL dimethylformamide was added dropwise over 10 minutes. After complete addition, the mixture was warmed to room temperature and stirred for 2.5 hours. The crude reaction mixture was slowly added to 50 mL of vigorously stirred ice water, resulting in a tan precipitate. The solid was collected by vacuum filtrates and washed three times with 20 mL water and three times with 20 mL of diethyl ether. The solid was dried in vacuo and triturated with ethyl acetate/hexane to yield 338 mg of 2-({[5-ethyl-1-(4-methoxybenzyl)-6-oxo-1,6-dihydropyrimidin-4-yl]oxy}methyl)benzonitrile as an off-white solid (53%): ¹H NMR (400 MHz, d₃-CH₃Cl) δ 1.09 (t, J=7.4 Hz, 3H), 2.55 (q, J=7.5 Hz, 2H), 3.78 (s, 3H), 5.00 (s, 2H), 5.53 (s, 2H), 6.84-6.89 (m, 2H), 7.24-7.29 (m, 2H), 7.40 (td, J=7.4, 1.7 Hz, 1H), 7.53-7.61 (m, 2H), 7.67 (d, J=7.6 Hz, 1H), 7.86 (s, 1H); LC/MS on 4.6×50 mm C-18 column, tᵣ=2.84 minutes (10 to 90% acetonitrile/water over 5 minutes at 4 ml/min with detection 220 nm, at 30° C.); ES-MS m/z 376 (M+H).

### Step 2: Preparation of 6-{[2-(aminomethyl)benzyl]oxy}-5-ethyl-3-(4-methoxybenzyl)pyrimidin-4(3H)-one

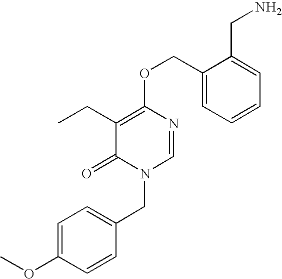

To a mixture of 336 mg of 2-{[(1-benzyl-5-ethyl-6-oxo-1,6-dihydropyrimidin-4-yl)oxy]methyl}benzonitrile (0.90 mmol) from step 1 above and 426 mg of cobalt chloride hexahydrate (1.8 mmol) in 5 mL of anhydrous methanol at 0 degrees Celsius was added portionwise 339 mg of sodium borohydride (9 mmol) over 30 minutes. Addition resulted in vigorous off-gassing and color change to black. The mixture was warmed to room temperature and stirred for 15 minutes, then 5 mL of 5% aqueous hydrogen chloride solution and 5 mL of water was added. The solution was basified by the addition of solid sodium carbonate to pH>9. The mixture was extracted twice with 50 mL methylene chloride, then the combined organic layers were dried over magnesium sulfate and concentrated in vacuo. The crude was dissolved in a mixture of 5 mL of methanol and 5 mL methylene chloride, and 1.76 g of polymer-bound sulfonic acid (Argonaut) was added to the jar was capped, and the mixture was agitated on a tabletop shaker for 1 hour. The resin was filtered and washed successively with methanol (3×50 mL) and methylene chloride (3×50 mL). The resin was treated with 5 mL of methylene chloride and 5 mL of 7 N ammonia in methanol. The mixture was agitated for 30 minutes on the tabletop shaker, then the resin was filtered and washed with successively with methanol (2×5 mL) and methylene chloride (2×5 mL). The resin was retreated with 5 mL of methylene chloride and 5 mL of 7 N ammonia in methanol for 30 minutes on the tabletop shaker, and the resin was again filtered and washed with successively with methanol (2×5 mL) and methylene chloride (2×5 mL). All filtrates were combined and concentrated in vacuo to yield 222 mg of 6-{[2-(aminomethyl)benzyl]oxy}-5-ethyl-3-(4-methoxybenzyl)pyrimidin-4(3H)-one as a tan solid (65%); LC/MS on 4.6×50 mm C-18 column, tᵣ=1.77 minutes (10 to 90% acetonitrile/water over 5 minutes at 4 ml/min with detection 220 nm, at 30° C.); ES-MS m/z 380 (M+H).

### Step 3: Preparation of 1-[3-tert-butyl-1-(4-methylphenyl)-1H-pyrazol-5-yl]-3-[2-({[5-ethyl-1-(4-methoxybenzyl)-6-oxo-1,6-dihydropyrimidin-4-yl]oxy}methyl)benzyl]urea

To a stirred solution of the 68 mg 6-{[2-(aminomethyl)benzyl]oxy}-5-ethyl-3-(4-methoxybenzyl)pyrimidin-4(3H)-one from step 2 above (0.2 mmol) and 54 uL of triethylamine (0.2 mmol) in 1 mL of methylene chloride was added 1 mL of the 0.2 M solution of 4-nitrophenyl [3-tert-butyl-1-(4-methylphenyl)-1H-pyrazol-5-yl]carbamate described in step 1 of 1-[2-({[3-bromo-1-(4-methoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea (0.1 mmol). The mixture was stirred at room temperature for 1 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 42 mg of 1-[3-tert-butyl-1-(4-methylphenyl)-1H-pyrazol-5-yl]-3-[2-({[5-ethyl-1-(4-methoxybenzyl)-6-oxo-1,6-dihydropyrimidin-4-yl]oxy}methyl)benzyl]urea as a white solid (35%): ¹H NMR (400 MHz, DMSO-d₆) δ 8.45 (1H, s), 8.11 (1H, s), 7.20-7.31 (10H, m), 6.82-6.89 (3H, m), 6.18 (1H, s), 5.34 (2H, s), 4.94 (2H, s), 4.27 (2H, d, J=5.5 Hz), 3.67 (3H, s), 2.26-2.32 (5H, m), 1.19 (9H, s), 0.88 (3H, t, J=7.3 Hz); LC/MS on 4.6×50 mm C-18 column, tᵣ=3.36 minutes (10 to 90% acetonitrile/water over 5 minutes at 4 ml/min with detection 220 nm, at 30° C.); ES-MS m/z 635 (M+H).

### Example 115

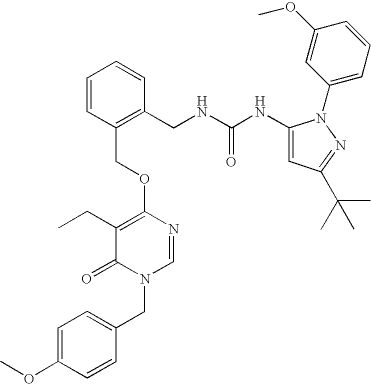

### 1-[3-tert-butyl-1-(3-methoxyphenyl)-1H-pyrazol-5-yl]-3-[2-({[5-ethyl-1-(4-methoxybenzyl)-6-oxo-1,6-dihydropyrimidin-4-yl]oxy}methyl)benzyl]urea

To a stirred solution of the 68 mg 6-{[2-(aminomethyl)benzyl]oxy}-5-ethyl-3-(4-methoxybenzyl)pyrimidin-4(3H)-one from step 2 of 1-[3-tert-butyl-1-(4-methylphenyl)-1H-pyrazol-5-yl]-3-[2-({[5-ethyl-1-(4-methoxybenzyl)-6-oxo-1,6-dihydropyrimidin-4-yl]oxy}methyl)benzyl]urea above (0.2 mmol) and 54 uL of triethylamine (0.2 mmol) in 1 mL of methylene chloride was added 1 mL of the 0.2 M solution of 0.2 M solution of 4-nitrophenyl [3-tert-butyl-1-(3-methoxyphenyl)-1H-pyrazol-5-yl]carbamate described in step 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-methoxyphenyl)-1H-pyrazol-5-yl]urea (0.2 mmol). The mixture was stirred at room temperature for 1 hours and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 28.4 mg of 1-[3-tert-butyl-1-(3-methoxyphenyl)-1H-pyrazol-5-yl]-3-[2-({[5-ethyl-1-(4-methoxybenzyl)-6-oxo-1,6-dihydropyrimidin-4-yl]oxy}methyl)benzyl]urea as a white solid (22%): ¹H NMR (400 MHz, DMSO-d₆) δ 8.45 (1H, s), 8.19 (1H, s), 7.32 (2H, t, J=8.2 Hz), 7.23 (5H, s), 6.96-7.01 (2H, m), 6.87-6.94 (2H, m), 6.84 (2H, d, J=8.4 Hz), 6.20 (1H, s), 5.35 (2H, s), 4.94 (2H, s), 4.28 (2H, d, J=5.5 Hz), 3.71 (3H, s), 3.66 (3H, s), 2.29 (2H, q, J=7.3 Hz), 1.19 (9H, s), 0.88 (3H, t, J=7.3 Hz); LC/MS on 4.6×50 mm C-18 column, tᵣ=3.29 minutes (10 to 90% acetonitrile/water over 5 minutes at 4 ml/min with detection 220 nm, at 30° C.); ES-MS m/z 651 (M+H).

### Example 116

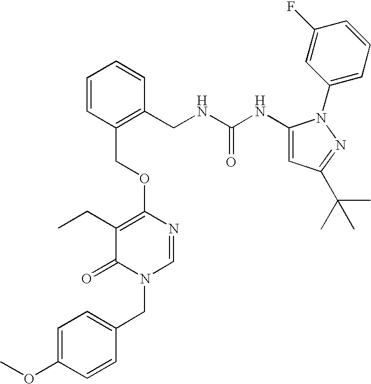

### 1-[3-tert-butyl-1-(3-fluorophenyl)-1H-pyrazol-5-yl]-3-[2-({[5-ethyl-1-(4-methoxybenzyl)-6-oxo-1,6-dihydropyrimidin-4-yl]oxy}methyl)benzyl]urea

To a stirred solution of the 68 mg 6-{[2-(aminomethyl)benzyl]oxy}-5-ethyl-3-(4-methoxybenzyl)pyrimidin-4(3H)-one from step 2 of 1-[3-tert-butyl-1-(4-methylphenyl)-1H-pyrazol-5-yl]-3-[2-({[5-ethyl-1-(4-methoxybenzyl)-6-oxo-1,6-dihydropyrimidin-4-yl]oxy}methyl)benzyl]urea above (0.2 mmol) and 54 uL of triethylamine (0.2 mmol) in 1 mL of methylene chloride was added 1 mL of the 0.2 M solution of 0.2 M solution of 4-nitrophenyl [3-tert-butyl-1-(3-methoxyphenyl)-1H-pyrazol-5-yl]carbamate described in step 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea (0.2 mmol). The mixture was stirred at room temperature for 1 hour and concentrated in vacuo. The residue was purified by preparative RP-HPLC on a 40×100 mm C-18 column (35 to 95% acetonitrile/water (0.1% trifluoroacetic acid) over 8 minutes at 70 ml/min with detection at 220 nm). Pure fractions were pooled and concentrated in vacuo. Neutralization of the resultant TFA salt was accomplished by dissolving in 1 mL of methanol and applying solution to a 0.2 g column of polymer-bound bicarbonate (Polymer Labs) and eluting with 25 mL methanol. The filtrate was concentrated in vacuo to give 70 mg of 1-[3-tert-butyl-1-(3-fluorophenyl)-1H-pyrazol-5-yl]-3-[2-({[5-ethyl-1-(4-methoxybenzyl)-6-oxo-1,6-dihydropyrimidin-4-yl]oxy}methyl)benzyl]urea as a white solid (56%): ¹H NMR (400 MHz, DMSO-d₆) δ 8.45 (1H, s), 8.27 (1H, s), 7.44 (1H, s), 7.28-7.32 (3H, m), 7.18-7.25 (5H, m), 7.12-7.17 (1H, m), 6.88-6.95 (1H, m), 6.84 (2H, d, J=8.4 Hz), 6.21 (1H, s), 5.34 (2H, s), 4.94 (2H, s), 4.28 (2H, d, J=5.9 Hz), 3.65-3.68 (3H, m), 2.25-2.31 (2H, m), 1.21 (9H, s), 0.88 (3H, t, J=7.3 Hz); LC/MS on 4.6×50 mm C-18 column, tᵣ=3.38 minutes (10 to 90% acetonitrile/water over 5 minutes at 4 ml/min with detection 220 nm, at 30° C.); ES-MS m/z 639 (M+H).

### Example 117

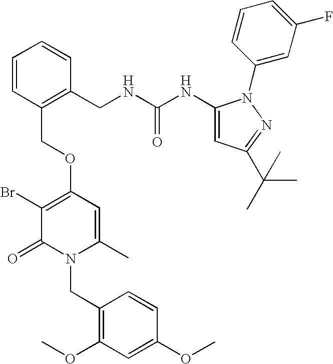

### 1-[2-({[3-bromo-1-(2,4-dimethoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea

### Step 1: Preparation of 2-({[1′-(2,4-dimethoxybenzyl)-6-methyl-2-oxo-1,2-dihydropyridin-4-yl]oxy}methyl)benzonitrile

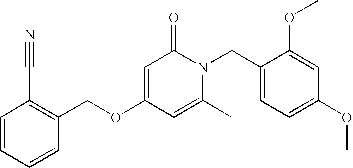

A solution of 25.0 g of 1-(2,4-dimethoxybenzyl)-4-hydroxy-6-methylpyridin-2(1H)-one (90.9 mmol, 14959-186), 19.6 g of alpha-bromotolunitrile (100 mmol) and 18.8 g of potassium carbonate (136.4 mmol) in 165 mL of anhydrous DMF was stirred at 65 C for 2 hours. After cooling to room temperature, the reaction was poured into 1 L of water and extracted with ethyl acetate (2×500 mL). The organic layer was washed with water (1×500 mL), 10% aqueous potassium carbonate (1×500 mL) and brine (1×500 mL), dried (MgSO₄) and concentrated to 38.6 g of a golden oil. Material is 4:1 desired product to a dibenzylation side product which was carried on as is.

### Step 2: Preparation of 2-({[3-bromo-1-(2,4-dimethoxybenzyl)-6-methyl-2-oxo-1,2-dihydropyridin-4-yl]oxy}methyl)benzonitrile

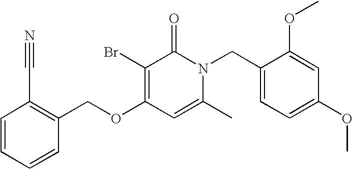

To a solution of 90.9 mmol of crude 2-({[1-(2,4-dimethoxybenzyl)-6-methyl-2-oxo-1,2-dihydropyridin-4-yl]oxy}methyl)benzonitrile in 180 mL of acetonitrile at 0 C was added 17.0 g of NBS (95.4 mmol) in one portion. After stirring at 0 C for 40 min, ice bath removed and reaction stirred at room temperature for 1.5 hours. Reaction was quenched with 20 mL of sat Na₂SO₃ and concentrated. Multiple recrystallizations from ethyl acetate/isopropanol followed by recrystallization from acetonitrile gave 6.57 g of product as an off white solid: ¹H NMR (400 MHz, DMSO-D6) δ ppm 2.29 (s, 3H), 3.74 (s, 3H), 3.85 (s, 3H), 5.14 (s, 2H), 5.45 (s, 2H), 6.45 (s, 2H), 6.60 (d, J=10.58 Hz, 2H), 7.62 (m, 1H), 7.81 (m, 2H), 7.96 (d, J=7.51 Hz, 1H). ES-MS m/z 469.06 (M+H).

### Step 3: Preparation of 4-{[2-(aminomethyl)benzyl]oxy}-3-bromo-1-(2,4-dimethoxybenzyl)-6-methylpyridin-2(1H)-one

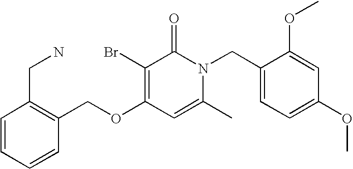

To a cooled suspension of 1.00 g of 2-({[3-bromo-1-(2,4-dimethoxybenzyl)-6-methyl-2-oxo-1,2-dihydropyridin-4-yl]oxy}methyl)benzonitrile (2.13 mmol) in 5.7 mL of anhydrous THF at 0 C was added 4.3 mL of 1 M BH3.THF (4.3 mmol) drop wise over 8 minutes. Upon completion of the addition, the ice bath was removed and the reaction stirred at room temperature overnight. The reaction was cooled and quenched with MeOH. After stirring 30 min, reaction concentrated to a white solid. The solid was dissolved in 35 mL of 1:1 MeOH:CH₂Cl₂ and treated with 8.7 g of MP-TsOH (4.0 eq/g) for 2 hours. Resin was filtered and washed with MeOH, CH₂Cl₂ 1:1 MeOH:CH₂Cl₂, CH₂Cl₂, and MeOH (50 mL of each). The resin was then agitated in 30 mL of 7N NH₃/MeOH for 2 hours. The resin was collected and washed as above. Concentration of the combined filtrate and washings gave 777.8 mg of the desired compound as a tan solid: ¹H NMR (400 MHz, DMSO-D6) δ ppm 2.23 (s, 3H), 3.70 (s, 3H), 3.79 (s, 2H), 3.81 (s, 3H), 5.10 (s, 2H), 5.34 (s, 2H), 6.41 (d, J=2 Hz, 2H), 6.54-6.59 (m, 2H), 7.24 (dt, J=7.6, 1.2 Hz, 1H), 7.32 (dt, J=7.6, 1.2 Hz, 1H), 7.44 (d, J=7.6 Hz, 2H). ES-MS m/z 473.07 (M+H).

### Step 4: Preparation of 1-[2-({[3-bromo-1-(2,4-dimethoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea

To a solution of 50 mg of 4-{[2-(aminomethyl)benzyl]oxy}-3-bromo-1-(2,4-dimethoxybenzyl)-6-methylpyridin-2(1H)-one (0.106 mmol) and 22.3 uL of triethylamine (0.16 mmol) in 1.5 mL of methylene chloride was added 0.55 mL of a 0.2 M solution of 4-nitrophenyl [3-tert-butyl-1-(3-fluorophenyl)-1H-pyrazol-5-yl]carbamate described in step 5 of 1-[2-({[3-bromo-1-(4-methoxybenzyl)-6-methyl-2-oxo-1,2-dihydropyridin-4-yl]oxy}methyl)benzyl]-3-[3-tert-butyl-1-(3-fluorophenyl)-1H-pyrazol-5-yl]urea (0.11 mmol). The reaction was stirred at room temperature for 30 minutes, concentrated and purified by RPHPLC. After concentration, compound was neutralized by filtering through MP-Carbonate resin. Concentration gave 34.3 mg of desired compound as a colorless solid: ¹H NMR (400 MHz, DMSO-D6) δ ppm 1.25 (s, 9H), 2.24 (s, 3H), 3.73 (s, 3H), 3.85 (s, 3H), 4.37 (m, 2H), 5.13 (s, 2H), 5.35 (s, 2H), 6.28 (s, 1H), 6.44 (m, 2H), 6.55 (m, 1H), 6.62 (m, 1H), 7.05 (m, 1H), 7.21 (m, 1H), 7.34 (m, 5H), 7.51 (m, 2H), 8,37 (m, 1H). ES-MS m/z 732(M+H). HRMS: Calc'd: 732.2191. Found: 732.2207

The following compounds were made in a similar manner as Example 117.

| Example | | | |
|---|---|---|---|
| Number | Compound Name | Calc'd | Found |
| 118 | 1-[2-({[3-bromo-1-(2,4-dimethoxybenzyl)-6-methyl- | 748.1896 | 748.1904 |
| | 2-oxo-1,2-dihydropyridin-4- | | |
| | yl]oxy}methyl)benzyl]-3-[3-tert-butyl-1-(3- | | |
| | chlorophenyl)-1H-pyrazol-5-yl]urea | | |
| 119 | 1-[2-({[3-bromo-1-(2,4-dimethoxybenzyl)-6- | 744.2391 | 744.2376 |
| | methyl-2-oxo-1,2-dihydropyridin-4- | | |
| | yl]oxy}methyl)benzyl]-3-[3-tert-butyl-1-(3- | | |
| | methoxyphenyl)-1H-pyrazol-5-yl]urea | | |
| 120 | 1-[2-({[3-bromo-1-(2,4-dimethoxybenzyl)-6- | 714.2286 | 714.2303 |
| | methyl-2-oxo-1,2-dihydropyridin-4- | | |
| | yl]oxy}methyl)benzyl]-3-(3-tert-butyl-1-phenyl- | | |
| | 1H-pyrazol-5-yl)urea | | |
| 121 | 1-[2-({[3-bromo-1-(2,4-dimethoxybenzyl)-6- | 750.2097 | 750.2100 |
| | methyl-2-oxo-1,2-dihydropyridin-4- | | |
| | yl]oxy}methyl)benzyl]-3-[3-tert-butyl-1-(2,4- | | |
| | difluorophenyl)-1H-pyrazol-5-yl]urea | | |
| 122 | 1-[2-({[3-bromo-1-(2,4-dimethoxybenzyl)-6- | 728.2442 | 728.2462 |
| | methyl-2-oxo-1,2-dihydropyridin-4- | | |
| | yl]oxy}methyl)benzyl]-3-[3-tert-butyl-1-(4- | | |
| | methylphenyl)-1H-pyrazol-5-yl]urea | | |

### Example 123

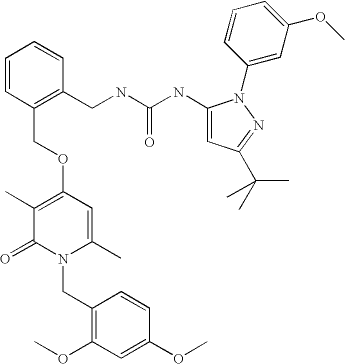

### 1-[3-tert-butyl-1-(3-methoxyphenyl)-1H-pyrazol-5-yl]-3-[2-({[1-(2,4-dimethoxybenzyl)-3,6-dimethyl-2-oxo-1,2-dihydropyridin-4-yl]oxy}methyl)benzyl]urea

### Step 1: Preparation of 2-({[1-(2,4-dimethoxybenzyl)-6-methyl-2-oxo-1,2-dihydropyridin-4-yl]oxy}methyl)benzonitrile

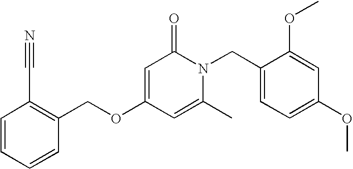

α-Bromotolunitrile (15.7 g, 80.0 mmol) was added slowly over 30 min to a stirring suspension of 20.0 g of 1-(2,4-dimethoxybenzyl)-4-hydroxy-6-methylpyridin-2(1H)-one (72.7 mmol, 14959-186) and 15.0 g of potassium carbonate (109.0 mmol) in 100 mL of anhydrous NMP at 60 C. Upon completion of addition, reaction stirred for 30 min at 60 C then cooled to room temperature. Reaction partitioned between water (1 L) and ethyl acetate (700 mL). Aqueous was extracted with ethyl acetate (200 mL). The combined organics were washed with 2N Na₂CO₃ (1×1 L), water (1×1 L) and brine (1×600 mL), dried (MgSO₄) and concentrated. Normal phase chromatography (50-90% ethyl acetate/hexane) followed by recrystallization from isopropanol multiple times gave 16.56 g of the desired compound as colorless crystals: ¹H NMR (400 MHz, DMSO-D6) δ ppm 2.13 (s, 3H), 3.70 (s, 3H), 3.81 (s, 3H), 4.99 (s, 2H), 5.20 (s, 2H), 5.91 (d, J=2.8 Hz, 1H), 5.96 (d, J=2.4 Hz, 1H), 6.41 (d, J=1.2 Hz, 2H), 6.57 (s, 1H), 7.57 (dt, J=7.6, 1.6 Hz, 1H), 7.70-7.77 (m, 2H), 7.90 (d, J=7.6 Hz, 1H). ES-MS m/z 391.17 (M+H).

### Step 2: Preparation of 2-({[1-(2,4-dimethoxybenzyl)-3-iodo-6-methyl-2-oxo-1,2-dihydropyridin-4-yl]oxy}methyl)benzonitrile

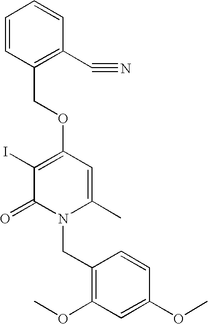

A solution of 5.0 g (12.8 mmol) of 2-({[1-(2,4-dimethoxybenzyl)-6-methyl-2-oxo-1,2-dihydropyridin-4-yl]oxy}methyl)benzonitrile, 3.0 g (13.4 mmol) N-iodosuccinimide and 0.26 mL of dichloroacetic acid (3.2 mmol) in 60 mL of anhydrous acetonitrile was stirred at 65 C for 3 hours, cooled and concentrated. Normal phase chromatography (0-10% ethyl acetate/methylene chloride) gave 2.71 g of the desired product as a colorless solid: ¹H NMR (400 MHz, DMSO-D6) δ ppm 2.26 (s, 3H), 3.70 (s, 3H), 3.81 (s, 3H), 5.12 (s, 2H), 5.40 (s, 2H), 6.40-6.42 (m, 2H), 6.58 (d, J=7.2 Hz, 1H), 7.55-7.60 (m, 1H), 7.77-7.79 (m, 2H), 7.91 (d, J=7.6 Hz, 1H).). ES-MS 7m/z 517.12 (M+H).

### Step 3: Preparation of 2-({[1-(2,4-dimethoxybenzyl)-3,6-dimethyl-2-oxo-1,2-dihydropyridin-4-yl]oxy}methyl)benzonitrile

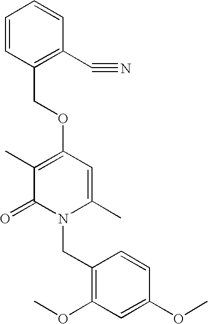

To a solution of 2.77 g of 2-({[1-(2,4-dimethoxybenzyl)-3-iodo-6-methyl-2-oxo-1,2-dihydropyridin-4-yl]oxy}methyl)benzonitrile (5.37 mmol), 0.80 g of LiCl (18.8 mmol) and 0.38 g of (Ph₃P)₂PdCl₂ (0.54 mmol) in 30 mL of anhydrous DMF under N₂ was added 1.6 mL of tetramethyltin (11.3 mmol). The reaction was heated to 85 C for 2 hours. After cooling, reaction diluted with water and extracted with ethyl acetate (2×50 mL). The combined organics were washed with brine (1×100 mL), dried (Na₂SO₄) and concentrated. Silica gel chromatography (0-30% ethyl acetate/CH₂Cl₂) gave 1.86 g of desired product as a colorless solid: ¹H NMR (400 MHz, DMSO-D6) δ ppm 1.88 (s, 3H), 2.23 (s, 3H), 3.73 (s, 3H), 3.85 (s, 3H), 5.09 (s, 2H), 5.34 (s, 2H), 6.36-6.45 (m, 3H), 6.60 (d, J=4 Hz, 1H), 7.60 (t, J=8 Hz, 1H), 7.73-7.82 (m, 2H), 7.94 (d, J=8 Hz, 1H). ES-MS m/z 405.23 (M+H).

### Step 4: Preparation of 4-{[2-(aminomethyl)benzyl]oxy}-1-(2,4-dimethoxybenzyl)-3,6-dimethylpyridin-2(1H)-one

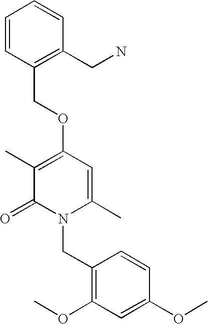

To a suspension of 0.50 g of 2-({[1-(2,4-dimethoxybenzyl)-3,6-dimethyl-2-oxo-1,2-dihydropyridin-4-yl]oxy}methyl)benzonitrile (1.24 mmol) in 3.1 mL of anhydrous THF under N₂ at 0 C was added 2.5 mL of 1M BH₃.THF (2.5 mmol) dropwise. Upon completion of the addition, the bath was removed and the reaction stirred at ambient temperature for 2 hours. The reaction was cooled, quenched by the addition of MeOH then concentrated. The residue was taken up in 10 mL of CH₂Cl₂ and 10 mL of MeOH and 3 g of MP-TsOH resin (4.07 mmol/g) was added. After stirring over night, resin was filtered and washed with 10 mL of CH₂Cl₂ and 10 mL of MeOH (3× each). The resin was suspended in 10 mL of CH₂Cl₂ and 10 mL of 7N NH₃/MeOH and stirred for 3 hours. The resin was filtered and washed with CH₂Cl₂, MeOH and 7N NH₃/MeOH. Concentration of the filtrate and washings gave 0.47 g of the desired compound as a colorless oil: ¹H NMR (400 MHz, DMSO-D6) δ ppm 1.87 (s, 3H), 2.22 (s, 3H), 3.17 (d, J=8 Hz, 2H), 3.73 (s, 3H), 3.81 (s, 2H), 3.85 (s, 3H), 5.09 (s, 2H), 5.27 (s, 2H), 6.36-6.45 (m, 3H), 6.61 (d, J=4 Hz, 1H), 7.25-7.36 (m, 2H), 7.43 (d, J=8 Hz, 1H), 7.48 (d, J=8 Hz, 1H). ES-MS m/z 409.26 (M+H).

### Step 5: Preparation of 4-nitrophenyl [3-tert-butyl-1-(3-methoxyphenyl)-1H-pyrazol-5-yl]carbamate

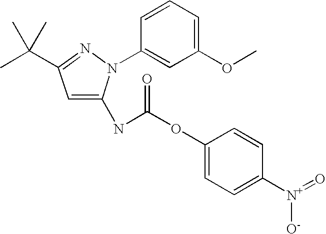

To a solution of 491 mg of 3-tert-butyl-1-(3-methoxyphenyl)-1H-pyrazol-5-amine (2.0 mmol) and 162 uL of pyridine (2.1 mmol) in 6 mL of anhydrous CHCl₃ was added 404 mg of 4-nitrophenylchloroformate (2.0 mmol). After stirring at ambient temperature over night, reaction mixture was filtered through 5 mL Chem-Elut tube prewetted with 4 mL of 5% HCl. Concentration gave 873.7 mg of the desired product as a foam. ES-MS m/z 411.19 (M+H).

### Step 6: Preparation of 1-[3-tert-butyl-1-(3-methoxyphenyl)-1H-pyrazol-5-yl]-3-[2-({[1-(2,4-dimethoxybenzyl)-3,6-dimethyl-2-oxo-1,2-dihydropyridin-4-yl]oxy}methyl)benzyl]urea

To 70.8 mg of 4-nitrophenyl [3-tert-butyl-1-(3-methoxyphenyl)-1H-pyrazol-5-yl]carbamates (0.172 mmol) in a vial was added a solution of 47 mg of 4-{[2-(aminomethyl)benzyl]oxy}-1-(2,4-dimethoxybenzyl)-3,6-dimethylpyridin-2(1H)-one (0.115 mmol) and 43.1 uL of triethylamine in 1 mL of CH₂Cl₂. The vial was capped, stirred for 15 minutes then concentrated. Reverse phase purification followed by neutralization with MP-CO₃ cartridges and concentration gave 9.6 mg of the desired product: ¹H NMR (400 MHz, DMSO-D6) δ ppm 1.43 (s, 9H), 2.06 (s, 3H), 2.37 (s, 3H), 3.90 (s, 3H), 3.95 (s, 3H), 4.03 (s, 3H) 4.54 (s, 2H), 5.26 (s, 2H), 5.42 (s, 2H), 6.45 (d, J=1.37 Hz, 1H), 6.52-6.64 (m, 3H), 6.78 (d, J=2.39 Hz, 1H), 7.10-7.16 (m, 1H), 7.19-7.27 (m, 3H), 7.43-7.61 (m, 4H), 7.62-7.68 (m, 1H), 8.45-8.50 (m, 1H). ES-MS m/z 680.38 (M+H).

The following compounds were made in a similar fashion as that of Example 123:

| Ex- | | | |
|---|---|---|---|
| ample | | | |
| Number | Compound Name | Calc'd | Found |
| 124 | 1-[3-tert-butyl-1-(3-fluorophenyl)-1H- | 668.3248 | 668.3247 |
| | pyrazol-5-yl]-3-[2-({[1-(2,4- | | |
| | dimethoxybenzyl)-3,6-dimethyl-2-oxo- | | |
| | 1,2-dihydropyridin-4- | | |
| | yl]oxy}methyl)benzyl]urea | | |
| 125 | 1-(3-tert-butyl-1-phenyl-1H-pyrazol-5- | 650.3342 | 650.3318 |
| | yl)-3-[2-({[1-(2,4-dimethoxybenzyl)- | | |
| | 3,6-dimethyl-2-oxo-1,2-dihydropyridin- | | |
| | 4-yl]oxy}methyl)benzyl]urea | | |
| 126 | 1-[3-tert-butyl-1-(2,4-difluorophenyl)- | 686.3154 | 686.3163 |
| | 1H-pyrazol-5-yl]-3-[2-({[1-(2,4- | | |
| | dimethoxybenzyl)-3,6-dimethyl-2-oxo- | | |
| | 1,2-dihydropyridin-4- | | |
| | yl]oxy}methyl)benzyl]urea | | |
| 127 | 1-[3-tert-butyl-1-(4-methylphenyl)-1H- | 664.3499 | 664.3506 |
| | pyrazol-5-yl]-3-[2-({[1-(2,4- | | |
| | dimethoxybenzyl)-3,6-dimethyl-2-oxo- | | |
| | 1,2-dihydropyridin-4- | | |
| | yl]oxy}methyl)benzyl]urea | | |

### Example 128

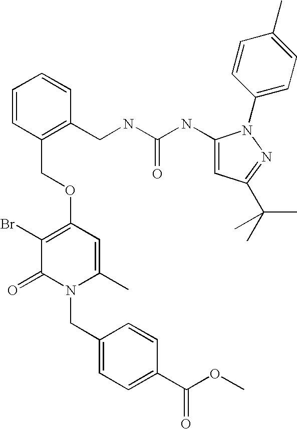

### Methyl 4-((4-(2-((3-(3-tert-butyl-1-p-tolyl-1H-pyrazol-5-yl)ureido)methyl)-benzyloxy)-3-bromo-6-methyl-2-oxopyridin-1(2H)-yl)methyl)benzoate

### Step 1: Preparation of 2-{[(3-bromo-6-methyl-2-oxo-1,2-dihydropyridin-4-yl)oxy]-methyl}benzonitrile

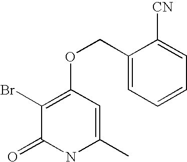

A suspension of 10.57 g of 2-({[3-bromo-1-(2,4-dimethoxybenzyl)-6-methyl-2-oxo-1,2-dihydropyridin-4-yl]oxy}methyl)benzonitrile (10.4 mmol) in 20 mL of trifluoroacetic acid, 4 mL of water and 4 mL of methanol was heated to 70 C for 2.5 hours. After cooling to ambient temperature, solid collected by filtration and washed with acetonitrile giving 2.45 g of the desired product as a pale purple solid: ¹H NMR (400 MHz, DMSO-D6) δ ppm 2.17 (s, 3H), 5.36 (s, 2H), 6.30 (s, 1H), 7.57 (dt, J=7.2, 1.6 Hz, 1H), 7.70-7.78 (m, 2H), 7.90 (d, J=7.6 Hz, 1H), 11.85 (bs, 1H). ES-MS m/z 319.00 (M+H).

### Step 2: Preparation of methyl 4-({3-bromo-4-[(2-cyanobenzyl)oxy]-6-methyl-2-oxopyridin-1(2H)-yl}methyl)benzoate

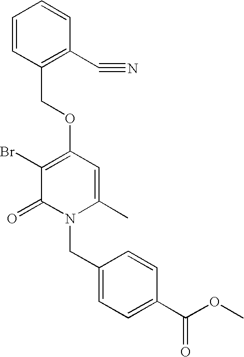

To a suspension of 2.4 g of 2-{[(3-bromo-6-methyl-2-oxo-1,2-dihydropyridin-4-yl)oxy]methyl}benzonitrile (7.5 mmol) and 2.6 g of methyl 4-(bromomethyl)benzoate (11.2 mmol) in anhydrous DMF was added 0.36 g of 60% sodium hydride (9.0 mmol) in one portion. After reaction had stopped off-gassing, it was heated to 50 C for 1 hour. Partitioned material between water and ethyl acetate, filtered off unreacted starting material as a fine purple powder, dried organic layer (MgSO₄) and concentrated. Alkylation isomers separated by RPHPLC, giving 659.7 mg of desired material as a colorless crystalline solid: ¹H NMR (400 MHz, DMSO-D6) δ ppm 2.28 (s, 3H), 3.81 (s, 3H), 5.37 (s, 2H), 5.41 (s, 2H), 6.58 (s, 1H), 7.21 (d, J=8.4 Hz, 2H), 7.58 (dt, J=7.6, 2 Hz, 1H), 7.36-7.82 (m, 2H), 7.90-7.93 (m, 3H). ES-MS m/z 467.13 (M+H).

### Step 3: Preparation of methyl 4-{[4-{[2-(aminomethyl)benzyl]oxy}-3-bromo-6-methyl-2-oxopyridin-1(2H)-yl]methyl}benzoate

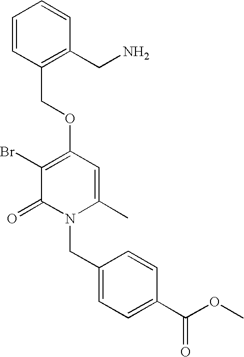

To a suspension of 100 mg of methyl 4-({3-bromo-4-[(2-cyanobenzyl)oxy]-6-methyl-2-oxopyridin-1(2H)-yl}methyl)benzoate (0.21 mmol) in 0.54 mL of anhydrous THF at 0° C. under N₂ was added 0.42 mL of 1M BH₃.THF dropwise. Upon completion of addition, reaction was stirred at 0° C. for 10 min then ambient temperature over night. Reaction cooled, quenched with MeOH and concentrated. The material was taken up in 2.8 mL of 1: I MeOH/CH₂Cl₂ and 0.52 g of MP-TsOH added (4.07 mmol/g). After mixing 6 hours, resin filtered and washed with MeOH and CH₂Cl₂ (3× each). The resin was suspended in 3 mL of CH₂Cl₂ and 3 mL of 7N NH₃/MeOH and stirred over night. The resin was filtered and washed with 7N NH₃/MeOH, CH₂Cl₂ and MeOH (2× each). Concentration of the filtrate and washings gave 84.7 mg of the desired product as a pale yellow oil: ES-MS m/z 471.12 (M+H).

### Step 4: Preparation of 1-[3-tert-butyl-1-(3-methoxyphenyl)-1H-pyrazol-5-yl]-3-[2-({[1-(4-methoxybenzyl)-3,6-dimethyl-2-oxo-1,2-dihydropyridin-4-yl]oxy}methyl)benzyl]urea

To a solution of 78.5 mg of methyl 4-{[4-{[2-(aminomethyl)benzyl]oxy}-3-bromo-6-methyl-2-oxopyridin-1(2H)-yl]methyl}benzoate (0.167 mmol) and 46.5 uL of triethylamine in 1 mL of anhydrous methylene chloride was added 99 mg of 4-nitrophenyl [3-tert-butyl-1-(4-methylphenyl)-1H-pyrazol-5-yl]carbamate. After 15 minutes, reaction was concentrated. RPHPLC and neutralization with MP-CO₃ cartridge followed by concentration gave 68.8 mg of the desired product as a colorless solid: ¹H NMR (400 MHz, DMSO-D6) δ ppm 1.17 (s, 9H), 2.20 (s, 3H), 2.29 (s, 3H), 3.78 (s, 3H), 4.30 (d, J=5.49 Hz, 2H), 5.29 (s, 2H), 5.33 (s, 2H), 6.17 (s, 1H), 6.49 (s, 1H), 6.93 (t, J=4.76 Hz, 1H), 7.11-7.40 (m, 9H), 7.44 (d, J=7.32 Hz, 1H), 7.88 (d, J=6.96 Hz, 2H), 8.14 (d, J=2.20 Hz, 1H). ES-MS m/z 725.22 (M+H). HRMS: Calc'd: 726.2291. Found: 726.2217.

### Example 128-B

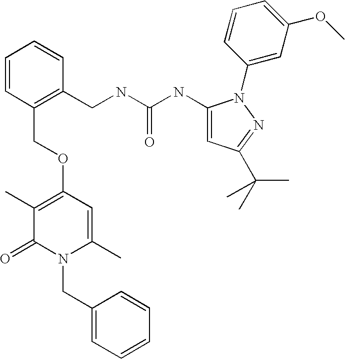

### 1-(2-{[(1-benzyl-3,6-dimethyl-2-oxo-1,2-dihydropyridin-4-yl)oxy]methyl}benzyl)-3-[3-tert-butyl-1-(3-methoxyphenyl)-1H-pyrazol-5-yl]urea

### Step 1: Preparation of 2-{[(3,6-dimethyl-2-oxo-1,2-dihydropyridin-4-yl)oxy]methyl}benzonitrile

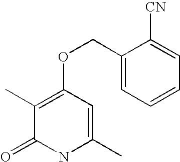

A solution of 1.34 g of 2-({[1-(2,4-dimethoxybenzyl)-3,6-dimethyl-2-oxo-1,2-dihydropyridin-4-yl]oxy}methyl)benzonitrile (3.32 mmol) in 6.6 mL of trifluoroacetic acid, 1.3 mL of water and 1.3 mL of methanol was stirred at 70 C for 2.5 hours. Material was cooled to ambient temperature and impurities filtered off. Crystallization from acetonitrile/methanol of concentrated filtrate gave 615.2 mg of the desired product as a colorless solid: ¹H NMR (400 MHz, DMSO-D6) δ ppm 1.79 (s, 3H), 2.16 (s, 3H), 5.29 (s, 2H), 6.17 (s, 1H), 7.58 (t, J=8 Hz, 1H), 7.68-7.82 (m, 2H), 7.93 (d, J=7.51 Hz, 1H), 11.37 (bs, 1H).). ES-MS m/z 255.17 (M+H).

### Step 2: Preparation of 2-{[(1-benzyl-3,6-dimethyl-2-oxo-1,2-dihydropyridin-4-yl)oxy]methyl}benzonitrile

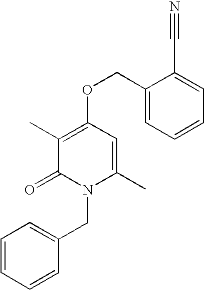

To a suspension of 218.8 mg of 2-{[(3,6-dimethyl-2-oxo-1,2-dihydropyridin-4-yl)oxy]methyl}benzonitrile (0.860 mmol) in 2 mL of anhydrous DMF was added 52 mg of 60% NaH (1.3 mmol). After stirring for 15 minutes, 123 uL of benzyl bromide (1.03 mmol) was added and reaction stirred over night. Reaction filtered through 20 mL Chem-elut tube prewetted with 15 mL of water and eluted with CH₂Cl₂ then concentrated. Normal phase chromatography (0-30% ethyl acetate/CH₂Cl₂) gave 109.1 mg of the desired product as a tan solid: ¹H NMR (400 MHz, DMSO-D6) δ ppm 1.90 (s, 3H), 2.27 (s, 3H), 5.29 (s, 2H), 5.33 (s, 2H), 6.41 (s, 1H), 7.09 (d, J=7.17 Hz, 2H), 7.25 (t, J=7.34 Hz, 1H), 7.33 (t, J=7.34 Hz, 2H), 7.60 (td, J=7.34, 1.71 Hz, 1H), 7.70-7.84 (m, 2H), 7.94 (d, J=7.51 Hz, 1H). ES-MS m/z 345.19 (M+H).

### Step 3: Preparation of 4-{[2-(aminomethyl)benzyl]oxy}-1-benzyl-3,6-dimethylpyridin-2(1H)-one

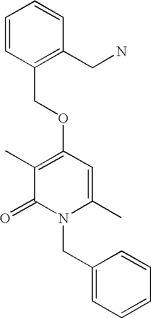

To a solution of 104.7 mg of 2-{[(1-benzyl-3,6-dimethyl-2-oxo-1,2-dihydropyridin-4-yl)oxy]methyl}benzonitrile (0.304 mmol) in 0.76 mL of anhydrous THF under N₂ at 0° C. was added 0.61 mL of 1M BH₃.THF (0.61 mmol) dropwise. Upon completion of addition, bath was removed and reaction stirred at ambient temperature for 1.5 hours. Reaction was cooled, quenched with MeOH and concentrated. Material dissolved in 3 mL of 1:1 MeOH:CH₂Cl₂, 0.75 g of MP-TsOH (4.07 mmol/g) added and mixture stirred ON. Resin was filtered, washed (with MeOH and CH₂Cl₂ 3× each), resuspended in 2 mL of CH₂Cl₂ and 2 mL of 7N NH₃/MeOH and stirred for 1 hour. Resin filtered and washed with MeOH, CH₂Cl₂ and 7N NH₃/MeOH (2× each). Concentration of filtrate and washings gave 96.2 mg of the desired product as a light brown oil: ES-MS m/z 349.23 (M+H).

### Step 4: Preparation of 1-(2-{[(1-benzyl-3,6-dimethyl-2-oxo-1,2-dihydropyridin-4-yl)oxy]methyl}benzyl)-3-[3-tert-butyl-1-(3-methoxyphenyl)-1H-pyrazol-5-yl]urea

To a solution of 48 mg of 4-{[2-(aminomethyl)benzyl]oxy}-1-benzyl-3,6-dimethylpyridin-2(1H)-one (0.138 mmol) and 52 uL of triethylamine (0.373 mmol) in 1 mL of anhyrdrous CH₂Cl₂ was added 85 mg of 4-nitrophenyl [3-tert-butyl-1-(3-methoxyphenyl)-1H-pyrazol-5-yl]carbamate (0.207 mmol). After stirring 15 minutes, reaction was concentrated. Reverse phase chromatography gave 36.2 mg of the desired product as a colorless powder: ¹H NMR (400 MHz, DMSO-D6) δ ppm 1.24 (s, 9H), 1.90 (s, 3H), 2.23 (s, 3H), 3.76 (s, 3H), 4.35 (d, J=5.49 Hz, 2H), 5.14-5.37 (m, 4H), 6.26 (s, 1H), 6.36 (s, 1H), 6.71-7.66 (m, 13H), 8.26 (s, 1H). ES-MS m/z 619.32 (M+H). HRMS: Calc'd: 620.3237. Found: 620.3073.

### Example 128-C

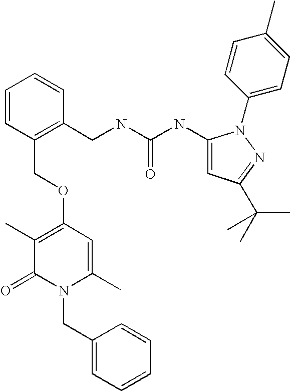

### 1-(2-{[(1-benzyl-3,6-dimethyl-2-oxo-1,2-dihydropyridin-4-yl)oxy]methyl}benzyl)-3-[3-tert-butyl-1-(4-methylphenyl)-1H-pyrazol-5-yl]urea

### Step 1: Preparation of 1-(2-{[(1-benzyl-3,6-dimethyl-2-oxo-1,2-dihydropyridin-4-yl)oxy]methyl}benzyl)-3-[3-tert-butyl-1-(4-methylphenyl)-1H-pyrazol-5-yl]urea

A solution of 48 mg of 4-{[2-(aminomethyl)benzyl]oxy}-1-benzyl-3,6-dimethylpyridin-2(1H)-one (0.138 mmol) and 52 uL of triethylamine (0.373 mmol) in 1 mL of anhyrdrous CH₂Cl₂ was added 82 mg of 4-nitrophenyl [3-tert-butyl-1-(4-methylphenyl)-1H-pyrazol-5-yl]carbamates (0.207 mmol). After stirring 15 minutes, reaction was concentrated. Reverse phase chromatography gave 34.1 mg of the desired product as a colorless powder: ¹H NMR (400 MHz, DMSO-D6) δ ppm 1.23 (s, 9H), 1.90 (s, 3H), 2.23 (s, 3H), 2.34 (s, 3H), 4.34 (d, J=5.49 Hz, 2H), 5.11-5.38 (m, 4H), 6.24 (s, 1H), 6.36 (s, 1H), 6.84-7.03 (m, 1H), 7.08 (d, J=7.32 Hz, 2H), 7.18-7.40 (m, 9H), 7.46 (d, J=6.59 Hz, 1H), 8.19 (s, 1H). ES-MS m/z 604.33 (M+H). HRMS: Calc'd: 604.3287. Found: 604.3271.

### Example 128-D

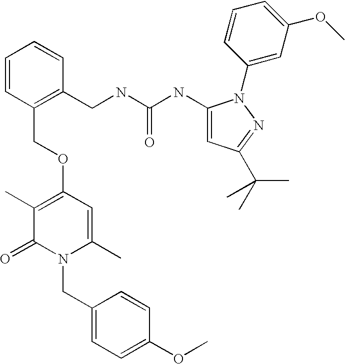

### 1-[3-tert-butyl-1-(3-methoxyphenyl)-1H-pyrazol-5-yl]-3-[2-({[1-(4-methoxybenzyl)-3,6-dimethyl-2-oxo-1,2-dihydropyridin-4-yl]oxy}methyl)benzyl]urea

### Step 1: Preparation of 2-({[1-(4-methoxybenzyl)-3,6-dimethyl-2-oxo-1,2-dihydropyridin-4-yl]oxy}methyl)benzonitrile

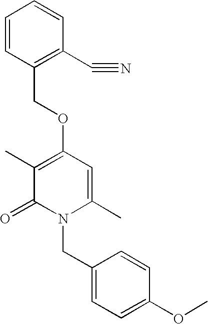

To a suspension of 456.6 mg of 2-{[(3,6-dimethyl-2-oxo-1,2-dihydropyridin-4-yl)oxy]methyl}benzonitrile (1.80 mmol) in 4 mL of anhydrous DMF was added 108 mg of 60% NaH (2.7 mmol). After stirring for 15 minutes, 294 uL of 4-methoxybenzyl chloride (2.16 mmol) was added and reaction stirred for 4 days. Crude reaction mixture diluted and purified by reverse phase chromatography giving 103.0 mg of the desired product as an off white solid: ¹H NMR (400 MHz, DMSO-D6) δ ppm 1.89 (s, 3H), 2.29 (s, 3H), 3.72 (s, 1H), 5.20 (s, 2H), 5.32 (s, 2H), 6.37 (s, 1H), 6.89 (d, J=8.88 Hz, 2H), 7.06 (d, J=8.88 Hz, 2H), 7.59 (dt, J=7.34, 1.54 Hz, 1H), 7.71-7.83 (m, 2H), 7.94 (d, J=7.51 Hz, 1H). ES-MS m/z 375.22 (M+H).

### Step 2: Preparation of 4-{[2-(aminomethyl)benzyl]oxy}-1-(4-methoxybenzyl)-3,6-dimethylpyridin-2(1H)-one

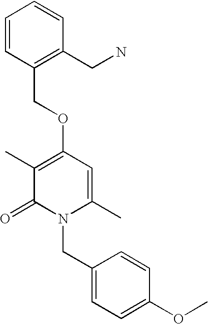

To a solution of 100.8 mg of 2-({[1-(4-methoxybenzyl)-3,6-dimethyl-2-oxo-1,2-dihydropyridin-4-yl]oxy}methyl)benzonitrile (0.269 mmol) in 0.7 mL of anhydrous THF at 0 C under N₂ added 0.54 mL of 1M BH₃.THF (0.54 mmol) drop wise. Upon completion of addition, bath removed and reaction stirred at ambient temperature for 30 minutes. Reaction cooled, quenched with MeOH and concentrated. Material dissolved in 3 mL of 1:1 MeOH:CH₂Cl₂, 0.66 g of MP-TsOH (4.07 mmol/g) added and mixture stirred ON. Resin filtered, washed (with MeOH and CH₂Cl₂ 3× each), resuspended in 2 mL of CH₂Cl₂ and 2 mL of 7N NH₃/MeOH and stirred for 1 hour. Resin filtered and washed with MeOH, CH₂Cl₂ and 7N NH₃/MeOH (2× each). Concentration of filtrate and washings gave 79.4 mg of the desired product as an oil: ES-MS m/z 379.26 (M+H).

### Step 3: Preparation of 1-[3-tert-butyl-1-(3-methoxyphenyl)-1H-pyrazol-5-yl]-3-[2-({[1-(4-methoxybenzyl)-3,6-dimethyl-2-oxo-1,2-dihydropyridin-4-yl]oxy}methyl)benzyl]urea

A solution of 39.5 mg of 4-{[2-(aminomethyl)benzyl]oxy}-1-(4-methoxybenzyl)-3,6-dimethylpyridin-2(1H)-one (0.105 mmol) and 40 uL of triethylamine (0.284 mmol) in 1 mL of anhydrous CH₂Cl₂ was added to 65 mg of 4-nitrophenyl [3-tert-butyl-1-(3-methoxyphenyl)-1H-pyrazol-5-yl]carbamate (0.158 mmol). After stirring 15 minutes, reaction was concentrated. Reverse phase chromatography gave 34.1 mg of the desired product as a colorless solid: ¹H NMR (400 MHz, DMSO-D6) δ ppm 1.24 (s, 9H), 1.89 (s, 3H), 2.24 (s, 3H), 3.71 (s, 3H), 3.75 (s, 3H), 4.34 (d, J=5.49 Hz, 2H), 5.08-5.30 (m, 4H), 6.25 (s, 1H), 6.33 (s, 1H), 6.87 (d, J=8.42 Hz, 2H), 6.94 (d, 1H), 6.97-7.12 (m, 5H), 7.20-7.40 (m, 4H), 7.45 (d, J=6.22 Hz, 1H), 8.27 (s, 1H). ES-MS m/z 650.44 (M+H). HRMS: Calc'd: 650.3342. Found: 650.3255.

### Example 129

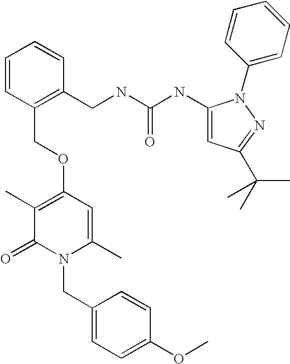

### 1-(3-tert-butyl-1-phenyl-1H-pyrazol-5-yl)-3-[2-({[1-(4-methoxybenzyl)-3,6-dimethyl-2-oxo-1,2-dihydropyridin-4-yl]oxy}methyl)benzyl]urea

A solution of 39.5 mg of 4-{[2-(aminomethyl)benzyl]oxy}-1-(4-methoxybenzyl)-3,6-dimethylpyridin-2(1H)-one (0.105 mmol) and 40 uL of triethylamine (0.284 mmol) in 1 mL of anhydrous CH₂Cl₂ was added to 60 mg of 4-nitrophenyl (3-tert-butyl-1-phenyl-1H-pyrazol-5-yl)carbamate (0.158 mmol). After stirring 15 minutes, reaction was concentrated. Reverse phase chromatography gave 30.6 mg of the desired product as a colorless solid: ¹H NMR (400 MHz, DMSO-D6) δ ppm 1.24 (s, 9H), 1.89 (s, 3H), 2.24 (s, 3H), 3.71 (s, 3H), 4.34 (d, J=5.49 Hz, 2H), 5.09-5.29 (m, 4H), 6.26 (s, 1H), 6.34 (s, 1H), 6.87 (d, J=8.42 Hz, 2H), 6.98 (t, J=5.67 Hz, 1H), 7.05 (d, J=8.79 Hz, 2H), 7.23-7.41 (m, 4H), 7.40-7.55 (m, 5H), 8.26 (s, 1H). ES-MS m/z 620.41 (M+H). HRMS: Calc'd: 620.3237. Found: 620.3104.

### Intermediate 51i

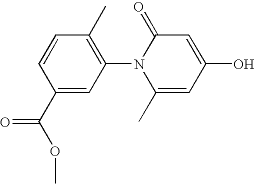

### Methyl 3-(4-hydroxy-6-methyl-2-oxopyridin-1(2H)-yl)-4-methylbenzoate

A suspension of 4-hydroxy-6-methyl-2H-pyran-2-one (1035 g, 8.21 mol), methyl 3-amino-4-methylbenzoate (900 g, 5.45 mol), and anhydrous potassium carbonate (90 g, 0.65 mol) in 2,2,2-trifluoroethanol (1800 mL) was heated at reflux for 22 hours. The temperature was lowered to 65° C. and the mixture was diluted with ethylacetate (14.9 L). The temperature was lowered further to 5-10° C. The mixture was filtered and the solid was washed with ethylacetate. The solid was dried on the filter. The product (891 g, 60%) was obtained as a light yellow solid.

### Intermediate 52i

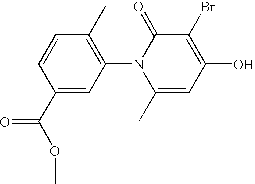

### Methyl 3-(3-bromo-4-hydroxy-6-methyl-2-oxopyridin-1(2H)-yl)-4-methylbenzoate

A suspension of methyl 3-(4-hydroxy-6-methyl-2-oxopyridin-1(2H)-yl)-4-methyl-benzoate (PHA744967) (10.0 g, 36.5 mmol) in methanol (40 mL) and acetic acid (10 mL) was chilled to 10° C. Bromine (6.1 g, 38 mmol) was added dropwise to the mixture in approximately 3 minutes while maintaining the temperature at 10-15° C. The mixture was stirred at ambient temperature for 15 minutes after the addition was completed. The mixture was treated with 10 wt % aqueous sodium metabisulfite (7.2 g) followed by water (50 mL). The mixture was stirred at ambient temperature for 15 minutes and then filtered. The solid was washed with aceto-nitrile (50 mL, slurry) followed by water (2×50 mL, slurry, displacement) and then dried on the filter. The product (11.6 g, 90%) was obtained as a white solid with a purity of 95 area %.

### Intermediate 53i

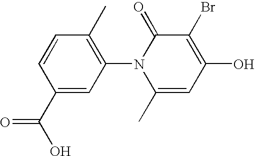

### (aS)-methyl 3-(3-chloro-4-hydroxy-6-methyl-2-oxopyridin-1(2H)-yl)-4-methylbenzoate

Methyl 3-(3-bromo-4-hydroxy-6-methyl-2-oxopyridin-1(2H)-yl)-4-methylbenzoate (1 kg, 2.84 moles) was mixed with 5 L of 1M Potassium Phosphate buffer solution and warmed to 30° C. The pH of the solution was adjusted to about 9.1 with 10% NaOH solution (about 1.3 L) followed by the addition of 400 mL Bacillus sp. Protease solution. After stirring for a total 48 hours, the pH of the solution was adjusted to 6.0 using 6N HCl solution over a period of 0.5 hours (about 880 mL) and stirred for another hour. At that point the undesired chiral ester methyl 3-(3-bromo-4-hydroxy-6-methyl-2-oxopyridin-1(2H)-yl)-4-methylbenzoate (423 gm) was isolated by filtration and washed with 700 mL of water. The aqueous solution was washed with 1.4 L methylene chloride. It was then further acidified to pH 3.5 with about 710 mL of 6N HCl to precipitate and isolate the chiral acid (aS)-3-(3-bromo-4-hydroxy-6-methyl-2-oxopyridin-1(2H)-yl)-4-methylbenzoic acid by filtration. 381 gm (40%) of product was obtained after drying.

### Intermediate 54i

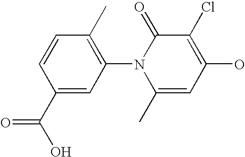

### (aS)-3-(3-chloro-4-hydroxy-6-methyl-2-oxopyridin-1(2H)-yl)-4-methylbenzoic acid

(aS)-3-(3-bromo-4-hydroxy-6-methyl-2-oxopyridin-1(2H)-yl)-4-methylbenzoic acid (1000 g, 2.96 moles) NMP (4400 g), and lithium chloride (878 g, 20.7 moles) were mixed and brought to 90° C. under nitrogen for 1 hour. After sampling to confirm complete reaction, water was slowly added to dissolve lithium bromide/chloride and to precipitate the product. The solution was gradually cooled to 5-10° C. An off-white solid was collected and washed with water to give about 90% isolated yield.

### Intermediate 55i

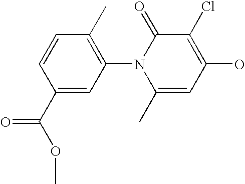

### (aS)-methyl 3-(3-chloro-4-hydroxy-6-methyl-2-oxopyridin-1(2H)-yl)-4-methylbenzoate

(aS)-3-(3-chloro-4-hydroxy-6-methyl-2-oxopyridin-1(2H)-yl)-4-methylbenzoic acid (90 g, 0.31 moles) and methanol (295 g) were mixed and cooled to 10° C. under nitrogen. Thionyl chloride (54.7 g, 0.46 moles) was added over 30 minutes while not exceeding 13° C., then the reaction was allowed to warm to ambient temperature (21-25° C.) and stirred for 24 hours. After sampling to confirm complete reaction, the reaction mixture was cooled to 10-15° C. and water (290 mL) was slowly added. A white solid was collected and washed with water to give about 95% isolated yield.

### Example 130

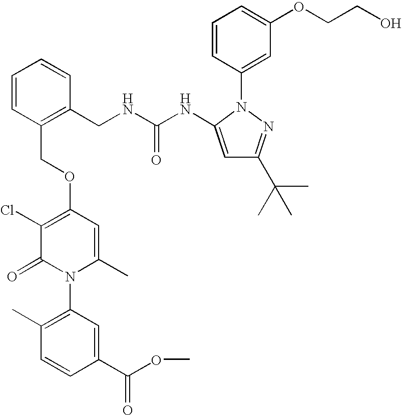

### 3-{4-[2-(3-{5-tert-Butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid methyl ester

### Step 1: Preparation of [3-chloro-4-(2-cyano-benzyloxy)-6-methyl-2-oxo-2H-pyridin-1-yl]-4-methyl-benzoic acid methyl ester

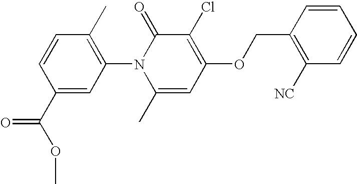

To a mixture of 3-[4-(2-cyano-benzyloxy)-6-methyl-2-oxo-2H-pyridin-1-yl]-4-methyl-benzoic acid methyl ester (2.00 g, 5.15 mmol) and N-chlorosuccinimide (0.701 g, 5.53 mmol) in THF (15 mL) was added a solution of p-toluenesulfonic acid monohydrate (0.049 g, 0.257 mmol) in methanol (4 mL) at room temperature. The resulting mixture was stirred overnight at 70° C., concentrated under vacuum and the residue was dissolved in ethyl acetate (20 mL), which was washed with saturated sodium bicarbonate (10 mL), brine (2×10 mL), dried over sodium sulfate then concentrated to dryness under reduced pressure. The residue obtained was purified by silica gel flash chromatography using ethyl acetate-hexanes (3:1) as eluant to give the desired product as white solid (1.963 g, 96.5%). ¹H NMR (CD₃OD/400 MHz): δ 8.06-8.03 (m, 1H), 7.84-7.75 (m, 4H), 7.59-7.54 (m, 2H), 6.71 (s, 1H), 5.50 (s, 2H), 3.89 (s, 3H), 2.10 (s, 3H), 1.98 (s, 3H); ES-MS m/z 422.94, 424.91 (C₂₃H₁₉ClN₂O₄ requires 422.87).

### Step 2: Preparation of 3-[4-(2-aminomethyl-benzyloxy)-3-chloro-6-methyl-2-oxo-2H-pyridin-1-yl]-4-methyl-benzoic acid methyl ester

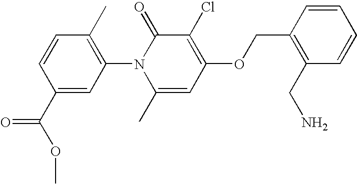

BH₃.THF (1M solution, 10.3 mL, 10.3 mmol) was added dropwise to a solution of [3-chloro-4-(2-cyano-benzyloxy)-6-methyl-2-oxo-2H-pyridin-1-yl]-4-methyl-benzoic acid methyl ester (1.75 g, 4.14 mmol) in THF (8 mL) at 0° C. under nitrogen. After stirring at this temperature for 30 min., the mixture was allowed to warm up to room temperature overnight, cooled to 0° C., quenched with methanol (3 mL) then concentrated to dryness under vacuum. The residue obtained was purified by silica gel flash chromatography using dichloromethane-methanol (20:3) as eluant to give the title compound as a white solid (0.996 g, 56.4%): ¹H NMR (CDCl₃/400 MHz) δ 8.05-8.02 (m, 1H), 7.77-7.75 (m, 1H), 7.48-7.26 (m, 5H), 6.28 (s, 1H), 5.37 (s, 2H), 3.89 (s, 3H), 2.14 (s, 3H), 1.93 (s, 3H), 1.86 (br, 2H); ES-MS m/z 427.11, 429.11 (C₂₃H₂₃ClN₂O₄ requires 426.90).

### Step 3: Preparation of 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-4-methyl-benzoic acid methyl ester

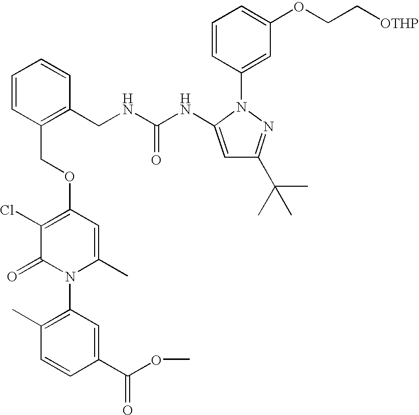

Phosgene (20% solution in toluene, 2.88 mL, 5.44 mmol) was added to a mixture of 5-tert-butyl-2-{3-[2-(tetrahydro-pyran-2-yloxy)-ethoxy]-phenyl}-2H-pyrazol-3-ylamine (0.977 g, 2.72 mmol), dichloromethane (48 mL) and saturated solution of NaHCO₃ (50 mL) at 0° C. After 15 min. most of the volatiles were removed under vacuum and the residue was dissolved in 5 mL of THF then treated with a solution of 3-[4-(2-aminomethyl-benzyloxy)-3-chloro-6-methyl-2-oxo-2H-pyridin-1-yl]-4-methyl-benzoic acid methyl ester (0.967 g, 2.26 mmol) in THF (5 mL) at 0° C. The resulting mixture was stirred at room temperature overnight (18 hr), concentrated under reduced pressure and the residue was purified by silica gel flash chromatography using ethyl acetate as elution to give the title compound (1.530 g, 83.2%). ¹H NMR (CDCl₃/400 MHz) δ 7.97-7.96 (m, 1H), 7.68 (s, 1H), 7.46-6.80 (m, 10H), 6.28-6.17 (m, 3H), 5.25 (s, 2H), 4.42 (d, 2H, J=5.6 Hz), 4.14-3.44 (m, 7H), 2.01 (s, 3H), 1.88 (s, 3H), 1.78-1.46 (m, 6H), 1.30 (s, 9H); ES-MS m/z 812.16 (C₄₄H₅₀ClN₅O₈ requires 812.36).

### Step 4: Preparation of 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid methyl ester

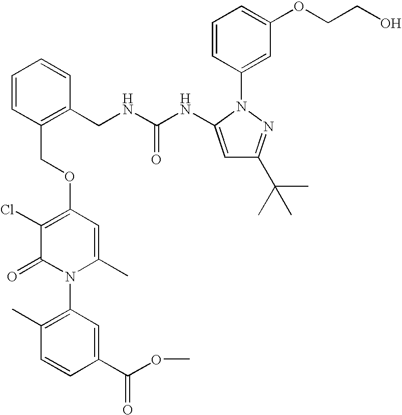

Pyridinium p-toluenesulfonate (0.142, 0.565 mmol) was added to the solution of 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-4-methyl-benzoic acid methyl ester (1.53 g, 1.88 mmol) in methanol (40 mL) and the mixture was stirred overnight at 50° C. The reaction mixture was concentrated under reduced pressure and the residue was purified by silica gel flash chromatography using ethyl acetate as elution to give the title compound as a white powder (1.21 g, 88.5%). M.p. 147-148° C.; ¹H NMR (CDCl₃/400 MHz) δ 8.01-7.99 (m, 1H), 7.71 (s, 1H), 7.48-7.22 (m, 7H), 6.97-6.76 (m, 4H), 6.37 (s, 1H), 6.27 (s, 1H), 5.92-5.89 (m, 1H), 5.27-5.20 (m, 2H), 4.51-4.41 (m, 2H), 3.97-3.94 (m, 2H), 3.89 (s, 3H), 3.79-3.76 (m, 3H), 2.95-2.96 (m, 1H), 2.05 (s, 3H), 1.92 (s, 3H), 1.32 (s, 9H); Anal. Calcd for C₃₉H₄₂ClN₅O₇-⅓EtOAc: C, 63.94; H, 5.94; N, 9.24. Found: C, 64.09; H, 5.94; N, 9.14; ES-MS⁻ m/z 728.19, 730.15 (C₃₉H₄₂ClN₅O₇ requires 728.24).

### Example 131

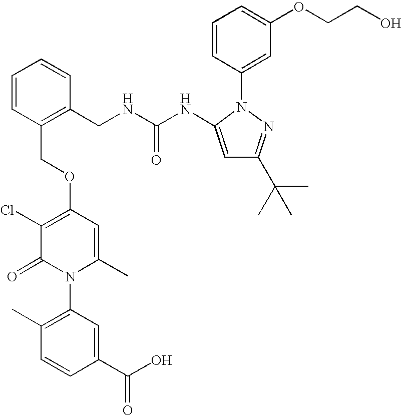

### 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid

To a solution of 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid methyl ester (1.0 g, 1.37 mmol) in THF (20 mL) at room temperature was added sodium hydroxide (0.55 g, 13.73 mmol). The mixture was stirred at 50° C. for 3 hours and concentrated. The residue obtained was neutralized with 1M citric acid to ˜pH 1 and the solid separated was filtered then dried to give desired product (0.834 g, 85.1%), which was used in the proceeding step without further purification. ¹H NMR (DMSO-d₆/400 MHz) δ 8.34, (s, 1H), 7.96-7.93 (m, 1H), 7.70 (s, 1H), 7.55-7.09 (m, 7H), 6.97-6.93 (m, 2H), 6.73 (s, 1H), 6.27 (s, 1H), 5.40 (s, 1H), 4.38 (d, 2H, J=5.8 Hz), 4.03-3.97 (m, 2H), 3.76-3.70 (m, 2H), 3.02 (s, 3H), 1.87 (s, 3H), 1.25 (s, 9H); ES-MS⁻ m/z 714.18, 716.16 (C₃₉H₄₀ClN₅O₇ requires 714.25).

### General Procedure G

To the solution of 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid in DMF (4-10 mL) was added EDCI (6 equiv) and the amine derivative at 0° C. under nitrogen. The mixture was allowed to warm up to room temperature while stirred overnight. After removal of most of the DMF, the residue was treated with ice-water (5 mL). The white solid was collected and by silica gel flash chromatography using dichloromethane/methanol (10:1) as eluant to give the desired product.

### Example 132

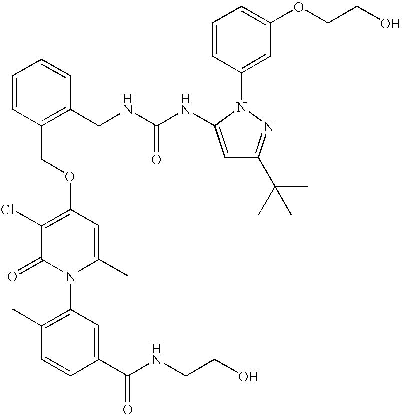

### 3-{4-[2-(3-{5-tert-Butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-N-(2-hydroxy-ethyl)-4-methyl-benzamide

This compound was synthesized according to General Procedure G from 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid (0.25 g, 0.35 mmol) and ethanolamine (0.043 g, 0.70 mmol) in the yield of 5.01 g (49.4%): M.p. 162-163° C.; ¹HNMR (DMSO-d₆/400 MHz) δ 8.45-8.43 (m, 1H), 8.30 (s, 1H), 7.88-7.87 (m, 1H), 7.65 (s, 1H), 7.52-7.50 (m, 2H), 7.40-7.30 (m, 4H), 7.05-7.04 (m, 3H), 6.97-6.94 (m, 1H), 6.74 (s, 1H), 6.27 (s, 1H), 5.41 (s, 2H), 4.88 (t, 1H, J=5.6 Hz), 4.73 (t, 1H, J=5.6 Hz), 4.39 (d, 2H, J=6.0 Hz), 4.03-4.00 (m, 2H), 3.74-3.70 (m, 2H), 3.52-3.48 (m, 2H), 3.36-3.62 (m, 2H), 2.01 (s, 3H), 1.90 (s, 3H), 1.25 (s, 9H); Anal. Calcd for C₄₀H₄₅ClN₆O₇. ⅓H₂O: C, 62.94; H, 6.03; N, 11.01. Found: C, 62.76; H, 6.06; N, 10.67; ES-MS m/z 757.10, 759.06 (C₄₀H₄₅ClN₆O₇ requires 757.28).

### Example 133

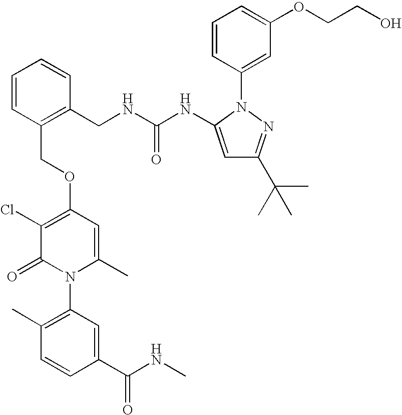

### 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4,N-dimethyl-benzamide

This compound was synthesized according to General Procedure G from 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid (0.25 g, 0.35 mmol) and methylamine (2.0M in THF, 0.7 mL, 0.70 mmol) in the yield of 0.118 g (46.4%): M.p. 168-170° C.; ¹H NMR (DMSO-d₆/400 MHz) δ 8.45-8.43 (m, 1H), 8.30 (s, 1H), 7.88-7.87 (m, 1H), 7.65 (s, 1H), 7.52-7.50 (m, 2H), 7.40-7.30 (m, 4H), 7.05-7.04 (m, 3H), 6.97-6.94 (m, 1H), 6.74 (s, 1H), 6.27 (s, 1H), 5.41 (s, 2H), 4.86 (t, 1H, J=5.6 Hz), 4.39 (d, 2H, J=6.0 Hz), 4.03-4.00 (m, 2H), 3.74-3.70 (m, 2H), 3.50 (s, 3H), 2.01 (s, 3H), 1.90 (s, 3H), 1.25 (s, 9H); Anal. Calcd for C₃₉H₄₃ClN₆O₆. ½ EtOAc: C, 63.85; H, 6.14; N, 10.90. Found: C, 63.36; H, 6.38; N, 10.61; ES-MS m/z 727.16, 729.20 (C₃₉H₄₃ClN₆O₆ requires 727.26).

### Example 134

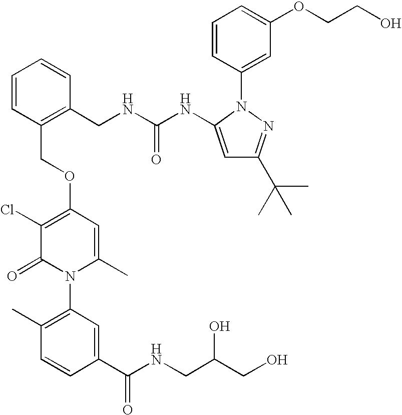

### 3-{4-[2-(3-{5-tert-Butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-N-(2,3-dihydroxy-propyl)-4-methyl-benzamide

This compound was synthesized according to General Procedure G from 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid (0.25 g, 0.35 mmol) and 3-amino-1,2-propanediol (0.064 g, 0.70 mmol) in the yield of 0.092 g (33.4%): M.p. 158-160° C.; ¹HNMR (DMSO-d₆/400 MHz) δ 8.45-8.43 (m, 1H), 8.30 (s, 1H), 7.88-7.87 (m, 1H), 7.65 (s, 1H), 7.52-7.50 (m, 2H), 7.40-7.30 (m, 4H), 7.05-7.04 (m, 3H), 6.97-6.94 (m, 1H), 6.74 (s, 1H), 6.27 (s, 1H), 5.41 (s, 2H), 4.88-4.79 (m, 2H), 4.55 (t, 1H, J=5.5 Hz), 4.39 (d, 2H, J=6.0 Hz), 4.03-4.00 (m, 2H), 3.74-3.70 (m, 2H), 3.58-3.06 (m, 5H), 2.01 (s, 3H), 1.90 (s, 3H), 1.25 (s, 9H); Anal. Calcd for C₄₁H₄₇ClN₆O₈-1 EtOAc: C, 61.74; H, 6.33; N, 9.60. Found: C, 61.23; H, 6.31; N, 9.72; ES-MS m/z 787.29, 789.18 (C₄₁H₄₇ClN₆O₈ requires 787.31).

### General Procedure H

A solution of 3-[4-(2-aminomethyl-benzyloxy)-3-chloro-6-methyl-2-oxo-2H-pyridin-1-yl]-4-methyl-benzoic acid methyl ester in dichloromethane was added to the solution of phosgene (20% in toluene, 6 equiv) in dichloromethane at 0° C. Then the saturated solution of sodium bicarbonate was added to the reaction mixture at 0° C. and the mixture was stirred for 15-20 minutes then the organic layer was dried over sodium sulfate. After most of the volatiles were removed in vacuo, the residue was dissolved in THF (25 mL and a solution of 3-(tert-butyl)-1-phenylpyrazole-5-yl-amine derivatives in THF (25 mL) was added to the mixture. The reaction mixture was stirred over night at room temperature and after removal of the volatiles in vacuo, the residue was purified by flash chromatography using ethyl acetate/hexanes/methanol (40:32:3) as eluant to give the desired product as a white solid.

### Example 135

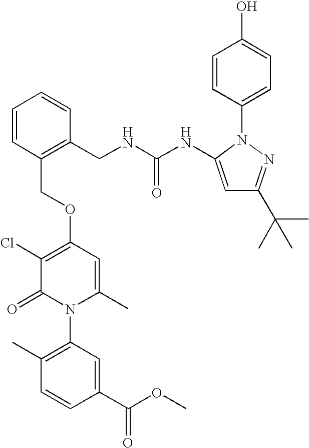

### 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid methyl ester

This compound was synthesized according to General Procedure H from 3-[4-(2-aminomethyl-benzyloxy)-3-chloro-6-methyl-2-oxo-2H-pyridin-1-yl]-4-methyl-benzoic acid methyl ester (2.0 g, 4.68 mmol), 4-(5-amino-3-tert-butyl-pyrazol-1-yl)-phenol (1.0 g, 4.26 mmol) in the yield of 2.732 g (93.7%): M.p. 199-200° C.; ¹H NMR (DMSO-d₆/400 MHz) δ 9.72 (s, 1H), 8.12 (s, 1H), 7.99-7.96 (m, 1H), 7.76 (s, 1H), 7.60-7.51 (m, 2H), 7.38-7.20 (m, 5H), 7.05-7.04 (m, 1H), 6.85-6.83 (m, 2H), 6.74 (s, 1H), 6.22 (s, 1H), 5.40 (s, 2H), 4.38 (d, 2H, J=5.6 Hz), 3.85 (s, 3H), 2.03 (s, 3H), 1.89 (s, 3H), 1.23 (s, 9H); Anal. Calcd for C₃₇H₃₈ClN₅O₆-1H₂O: C, 63.28; H, 5.74; N, 9.97. Found: C, 63.18; H, 5.46; N, 9.73; ES-MS m/z 684.29, 686.26 (C₃₇H₃₈ClN₅O₆ requires 684.69).

### Example 136

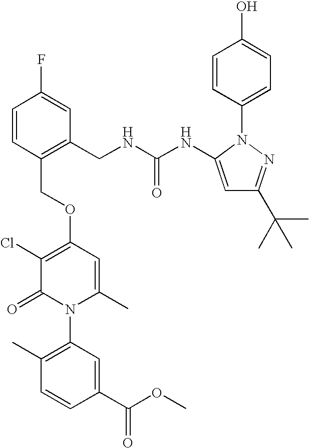

### 3-[4-(2-{3-[5-tert-butyl-2-(4-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid methyl ester

This compound was synthesized according to General Procedure H from 3-[4-(2-aminomethyl-4-fluoro-benzyloxy)-3-chloro-6-methyl-2-oxo-2H-pyridin-1-yl]-4-methyl-benzoic acid methyl ester (1.0 g, 2.247 mmol), 4-(5-amino-3-tert-butyl-pyrazol-1-yl)-phenol (0.6 g, 2.62 mmol) in the yield of 0.30 g (20.2%): M.p. 188-190° C. ¹H NMR (DMSO-d₆/400 MHz) δ 9.72 (s, 1H), 8.19 (s, 1H), 8.0 (m, 1H), 7.78 (s, 1H), 7.6 (m, 2H), 7.22-6.88 (m, 9H), 6.2 (s, 1H), 5.37 (s, 2H), 4.39 (m, 2H), 3.8 (s, 3H), 2.01 (s, 3H), 1.78 (s, 3H), 1.2 (s, 9H); Anal. Calcd for C₃₇H₃₇ClFNO₆: C, 63.29; H, 5.31; N, 9.97. Found: C, 63.00; H, 5.36; N, 9.89 ES-MS m/z 702.14, 704.11 (C₃₇H₃₇ClFNO₆ requires 702.19).

### Example 137

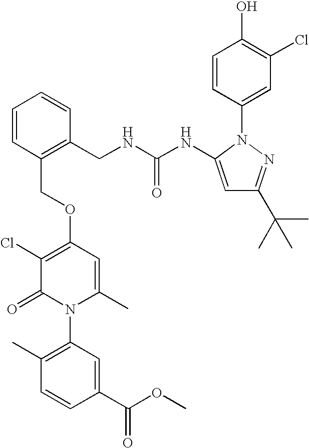

### 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid methyl ester

This compound was synthesized according to General Procedure H from 3-[4-(2-aminomethyl-benzyloxy)-3-chloro-6-methyl-2-oxo-2H-pyridin-1-yl]-4-methyl-benzoic acid methyl ester (1.5 g, 3.51 mmol), 4-(5-amino-3-tert-butyl-pyrazol-1-yl)-2-chloro-phenol (1.0 g, 4.21 mmol) in the yield of 2.872 g (57.0%): M.p. 185-186° C.;

¹H NMR (DMSO-d₆/400 MHz) δ 10.49 (s, 1H), 8.20 (s, 1H), 7.99-7.96 (m, 1H), 7.76 (s, 1H), 7.60-7.22 (m, 6H), 7.05-6.98 (m, 2H), 6.73 (s, 1H), 6.22 (s, 1H), 5.40 (s, 2H), 4.38 (d, 2H, J=5.6 Hz), 3.85 (s, 3H), 2.03 (s, 3H), 1.89 (s, 3H), 1.23 (s, 9H); Anal. Calcd for C₃₇H₃₇Cl₂N₅O₆: C, 61.84; H, 5.19; N, 9.74. Found: C, 62.31; H, 5.26; N, 9.51; ES-MS m/z 718.18, 720.19 (C₃₇H₃₇Cl₂N₅O₆ requires 718.63).

### Example 138

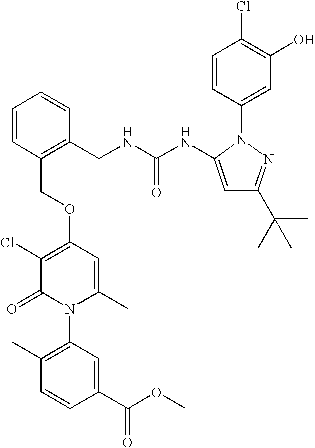

### 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-benzoic acid methyl ester

This compound was synthesized according to General Procedure H from 3-[4-(2-aminomethyl-benzyloxy)-3-chloro-6-methyl-2-oxo-2H-pyridin-1-yl]-4-methyl-benzoic acid methyl ester (1.5 g, 3.51 mmol), 3-(5-amino-3-tert-butyl-pyrazol-1-yl)-4-chloro-phenol (1.12 g, 4.21 mmol) in the yield of 2.791 g (55.3%): M.p. 193-195° C.;

¹H NMR (DMSO-d₆/400 MHz) δ 10.53 (s, 1H), 8.31 (s, 1H), 7.98-7.96 (m, 1H), 7.76 (s, 1H), 7.58-7.52 (m, 2H), 7.41-7.30 (m, 4H), 7.13-6.91 (m, 3H), 6.73 (s, 1H), 6.26 (s, 1H), 5.40 (s, 2H), 4.38 (d, 2H, J=5.6 Hz), 3.85 (s, 3H), 2.03 (s, 3H), 1.89 (s, 3H), 1.23 (s, 9H); Anal. Calcd for C₃₇H₃₇Cl₂N₅O₆: C, 61.84; H, 5.19; N, 9.74. Found: C, 61.64; H, 5.17; N, 9.67; ES-MS m/z 718.03, 720.24 (C₃₇H₃₇Cl₂N₅O₆ requires 718.63).

### Example 139

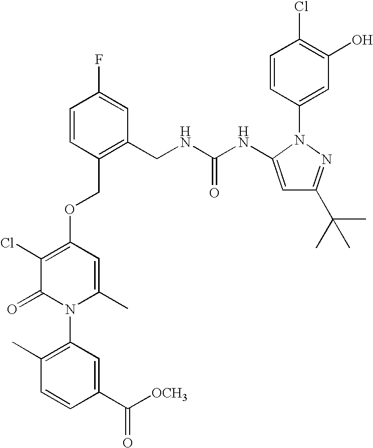

### 3-[4-(2-{3-[5-tert-butyl-2-(4-chloro-3-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid methyl ester

This compound was synthesized according to General Procedure H from 3-[4-(2-Aminomethyl-4-fluoro-benzyloxy)-3-chloro-6-methyl-2-oxo-2H-pyridin-1-yl]-4-methyl-benzoic acid methyl ester (1.99 g, 4.50 mmol), 3-(5-amino-3-tert-butyl-pyrazol-1-yl)-4-chloro-phenol (1.20 g, 4.50 mmol) in the yield of 2.0 g (60.4%): ¹H NMR (CD₃OD/400 MHz) δ 8.05-8.00 (d, 1H, J=5.6), 7.79 (s, 1H), 7.58-7.50 (m, 2H), 7.35-7.30 (d, 1H, J=5.6 Hz), 7.10-7.00 (m, 3H), 6.90-6.84 (d, 1H, J=5.6 Hz), 6.62 (s, 1H), 6.13 (s, 1H), 5.32 (s, 2H), 4.42 (s, 2H), 3.84 (s, 3H), 2.08 (s, 3H), 1.97 (s, 3H), 1.25 (s, 9H).

### Example 140

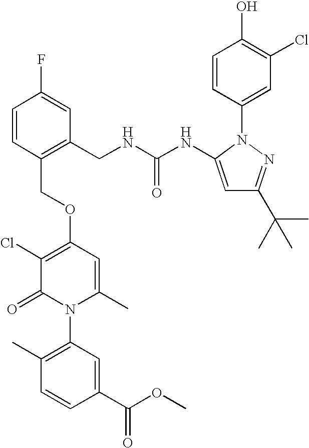

### 3-[4-(2-{3-[5-tert-butyl-2-(3-chloro-4-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid methyl ester

This compound was synthesized according to General Procedure H from 3-[4-(2-aminomethyl-4-fluoro-benzyloxy)-3-chloro-6-methyl-2-oxo-2H-pyridin-1-yl]-4-methyl-benzoic acid methyl ester (3.0 g, 7.19 mmol), 4-(5-amino-3-tert-butyl-pyrazol-1-yl)-2-chloro-phenol (1.90 g, 7.15 mmol) in the yield of 2.15 g (43.3%): M.p. 197-200° C. ¹H NMR (CD₃OD/400 MHz) δ 8.05 (m, 1H), 7.8 (s, 1H), 7.55 (m, 2H), 7.4 (s, 1H), 7.2-6.9 (m, 5H), 6.65 (s, 1H,), 6.22 (s, 1H), 5.35 (s, 2H), 4.42 (m, 2H), 3.85 (s, 3H), 2.1 (s, 3H), 1.22 (s, 9H); Anal. Calcd. For C₃₇H₃₆Cl₂FN₅O₆: C, 60.33; H, 4.99; N, 9.51. Found: C, 60.38; H, 4.79; N, 9.32; ES-MS m/z 738.02, 739.02, 736.06 (C₃₇H₃₆Cl₂FN₅O₆ requires 736.63).

### General Procedure I

To a solution of 3-[4-(2-{3-[5-tert-butyl-2-phenyl-2H-pyrazol-3-yl]-ureidomethyl}-benzyloxy)-3-chloro-6-methyl-2-oxo-2H-pyridin-1-yl]-4-methyl-benzoic acid methyl ester in ethanol was added lithium hydroxide (10 equiv.) in water (1M) at 0° C. The mixture was stirred at room temperature overnight and concentrated. The residue obtained was neutralized with 1M citric acid to ˜pH 1 and the solid separated was filtered then dried to give the desired product, which can be used without further purification.

### Example 141

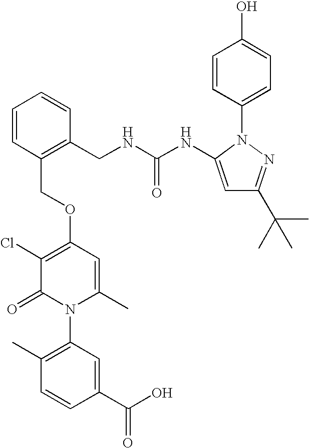

### 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid

This compound was synthesized according to General Procedure C from 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid methyl ester (2.617 g, 4.26 mmol) in the yield of 2.362 g (92.2%): ¹H NMR (DMSO-d₆/400 MHz) δ 13.08 (br, 1H), 9.71 (s, 1H), 8.10 (s, 1H), 7.99-7.96 (m, 1H), 7.76 (s, 1H), 7.60-7.51 (m, 2H), 7.38-7.20 (m, 5H), 7.05-7.04 (m, 1H), 6.85-6.83 (m, 2H), 6.74 (s, 1H), 6.22 (s, 1H), 5.40 (s, 2H), 4.38 (d, 2H, J=6.0 Hz), 2.03 (s, 3H), 1.89 (s, 3H), 1.23 (s, 9H).

### Example 142

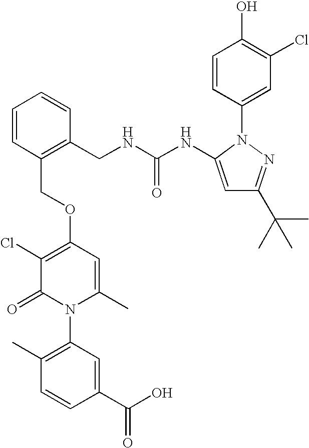

### 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid

This compound was synthesized according to General Procedure I from 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid methyl ester (2.809 g, 3.901 mmol) in the yield of 2.731 g (99.2%): ¹H NMR (DMSO-d₆/400 MHz) δ 10.49 (s, 1H), 8.20 (s, 1H), 7.99-7.96 (m, 1H), 7.76 (s, 1H), 7.60-7.22 (m, 6H), 7.05-6.98 (m, 2H), 6.73 (s, 1H), 6.22 (s, 1H), 5.40 (s, 2H), 4.38 (d, 2H, J=5.6 Hz), 2.02 (s, 3H), 1.89 (s, 3H), 1.23 (s, 9H); ES-MS m/z 704.19, 706.06 (C₃₆H₃₅Cl₂N₅O₆ requires 704.61).

### Example 143

This compound was synthesized according to General Procedure I from 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-benzoic acid methyl ester (2.720 g, 3.785 mmol) in the yield of 2.298 g (86.2%): ¹H NMR (DMSO-d₆/400 MHz) δ 10.55 (s, 1H), 8.31 (s, 1H), 7.98-7.96 (m, 1H), 7.76 (s, 1H), 7.58-7.52 (m, 2H), 7.41-7.30 (m, 4H), 7.13-6.91 (m, 3H), 6.73 (s, 1H), 6.26 (s, 1H), 5.40 (s, 2H), 4.38 (d, 2H, J=5.6 Hz), 2.03 (s, 3H), 1.89 (s, 3H), 1.23 (s, 9H); ES-MS m/z 703.99, 706.17 (C₃₆H₃₅Cl₂N₅O₆ requires 704.61).

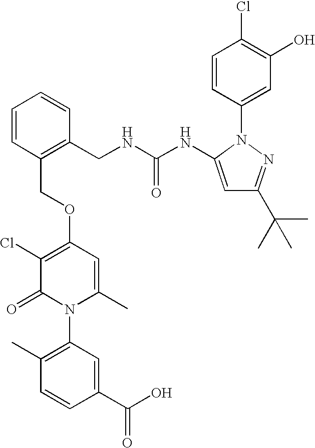

### 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-benzoic acid

### Example 144

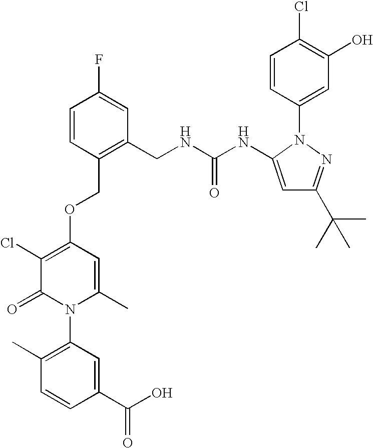

### 3-[4-(2-{3-[5-tert-butyl-2-(4-chloro-3-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid

This compound was synthesized according to General Procedure I from 3-[4-(2-{3-[5-tert-butyl-2-(4-chloro-3-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid methyl ester (1.86 g, 2.525 mmol) in the yield of 1.8 g (98.6%): ¹H NMR (DMSO-d₆/400 MHz) δ 10.58 (s, 1H), 8.40 (s, 1H), 7.98 (d, 1H, J=5.6 Hz), 7.74 (s, 1H), 7.61-7.51 (m, 2H), 7.39 (d, 1H, J=5.6 Hz), 7.20-7.00 (m, 2H), 6.90 (d, 1H, J=5.6 Hz); 6.72 (s, 1H); 6.25 (s, 1H), 5.40 (s, 2H), 4.40 (d, 2H, J=5.6 Hz), 2.03 (s, 3H), 1.89 (s, 3H), 1.23 (s, 9H); ES-MS m/z 721.98, 724.15 (C₃₆H₃₄Cl₂FN₅O₆ requires 722.61).

### Example 146

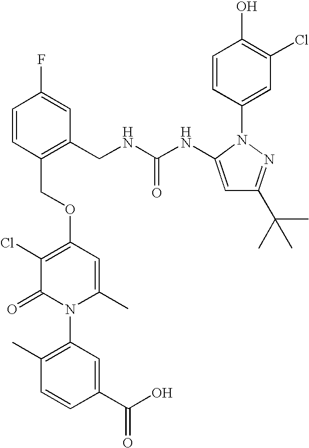

### 3-[4-(2-{3-[5-tert-butyl-2-(3-chloro-4-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid

This compound was synthesized according to General Procedure I from 3-[4-(2-{3-[5-tert-butyl-2-(3-chloro-4-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid methyl ester (1.85 g, 2.51 mmol) in the yield of 1.6 g (88.0%): ¹H NMR (DMSO-d₆/400 MHz) δ 10.5 (s, 1H), 8.3 (s, 1H), 7.95-6.65 (m, 9H), 6.2 (s, 1H), 5.4 (m, 2H), 4.4 (m, 2H), 2.1 (s, 3H), 1.95 (s, 3H), 1.25 (s, 9H,), ES-MS m/z 721.99, 723.90 (C₃₆H₃₄Cl₂FN₅O₆ requires 722.61).

### General Procedure J

To the solution of 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid in DMF (4-10 mL) was added EDCI (6 equiv) or CDI (3 equiv) followed by the amine derivative at 0° C. under nitrogen. The mixture was allowed to warm up to room temperature and stirred overnight. After removal of most of the DMF, the residue was treated with ice-water (5 mL) and the white solid was collected, which was purified by silica gel flash chromatography using dichloromethane/methanol (10:1) as eluant to give the desired product.

### Example 147

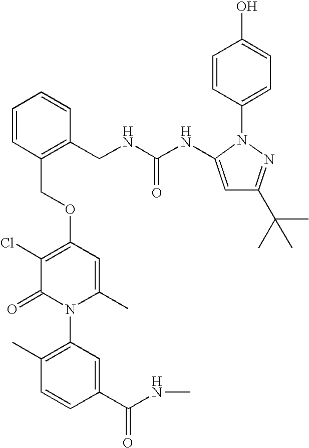

### 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4,N-dimethyl-benzamide

This compound was synthesized according to General Procedure J from 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.373 mmol), methylamine (2M in THF, 0.7 mL, 1.40 mmol) and EDCI (0.429 g, 2.24 mmol) in the yield of 0.141 g (55.3%): ¹HNMR (DMSO-d₆/400 MHz) δ 9.71 (s, 1H), 8.44-8.42 (m, 1H), 8.10 (s, 1H), 7.86-7.84 (m, 1H), 7.60 (s, 1H), 7.53-7.52 (m, 2H), 7.38-7.20 (m, 5H), 7.05-7.04 (m, 1H), 6.85-6.83 (m, 2H), 6.73 (s, 1H), 6.22 (s, 1H), 5.40 (s, 2H), 4.38 (d, 2H, J=6.0 Hz), 2.73 (d, 2H, J=3.2 Hz), 2.00 (s, 3H), 1.89 (s, 3H), 1.23 (s, 9H); Anal. Calcd for C₃₇H₃₉ClN₅O₆: C, 65.05; H, 5.75; N, 12.30. Found: C, 65.39; H, 5.74; N, 12.16; ES-MS m/z 683.11, 685.10 (C₃₇H₃₉ClN₅O₆ requires 683.20).

### Example 148

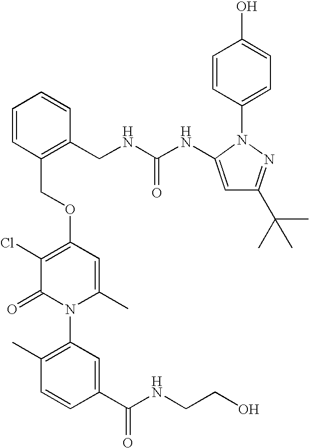

### 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-N-(2-hydroxy-ethyl)-4-methyl-benzamide

This compound was synthesized according to General Procedure J from 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.373 mmol), ethanolamine (0.046 g, 0.746 mmol), and EDCI (0.429 g, 2.24 mmol) in the yield of 0.136 g (51.1%): ¹H NMR (DMSO-d₆/400 MHz) δ 9.71 (s, 1H), 8.44-8.42 (m, 1H), 8.10 (s, 1H), 7.86-7.84 (m, 1H), 7.60 (s, 1H), 7.53-7.52 (m, 2H), 7.38-7.20 (m, 5H), 7.05-7.04 (m, 1H), 6.85-6.83 (m, 2H), 6.73 (s, 1H), 6.22 (s, 1H), 5.41 (s, 2H), 4.75-4.72 (m, 1H), 4.38 (d, 2H, J=5.6 Hz), 3.52-3.26 (m, 4H), 2.00 (s, 3H), 1.89 (s, 3H), 1.23 (s, 9H); Anal. Calcd for C₃₈H₄₁ClN₆O₆-½EtOAc: C, 63.44; H, 5.99; N, 11.10. Found: C, 63.36; H, 5.98; N, 11.10; ES-MS m/z 713.10, 715.09 (C₃₈H₄₁ClN₆O₆ requires 713.23).

### Example 149

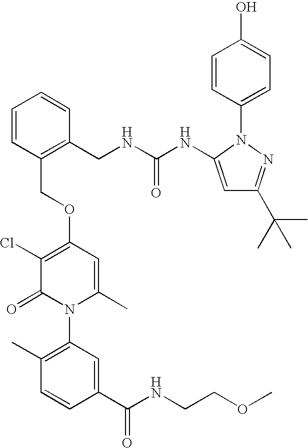

### 3-[4-(2-{3-[5-tert-Butyl-2-(4-hydroxy-phenyl)-2H-pyrazol-3-yl]-ureidomethyl}-benzyloxy)-3-chloro-6-methyl-2-oxo-2H-pyridin-1-yl]-N-(2-methoxy-ethyl)-4-methyl-benzamide

This compound was synthesized according to General Procedure J from 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.30 g, 0.448 mmol), 2-methoxyethanolamine (0.134 g, 1.79 mmol), and EDCI (0.515 g, 2.69 mmol) in the yield of 0.176 g (54.1%): ¹H NMR (DMSO-d₆/400 MHz) δ 9.73 (s, 1H), 8.44-8.42 (m, 1H), 8.10 (s, 1H), 7.86-7.84 (m, 1H), 7.60 (s, 1H), 7.53-7.52 (m, 2H), 7.38-7.20 (m, 5H), 7.05-7.04 (m, 1H), 6.85-6.83 (m, 2H), 6.73 (s, 1H), 6.22 (s, 1H), 5.41 (s, 2H), 4.38 (d, 2H, J=5.6 Hz), 3.47-3.26 (m, 7H), 2.00 (s, 3H), 1.89 (s, 3H), 1.23 (s, 9H); Anal. Calcd for C₃₉H₄₃ClN₆O₆: C, 64.41; H, 5.96; N, 11.56. Found: C, 64.57; H, 5.85; N, 11.17; ES-MS m/z 727.12, 729.11 (C₃₉H₄₃ClN₆O₆ requires 727.26).

### Example 150

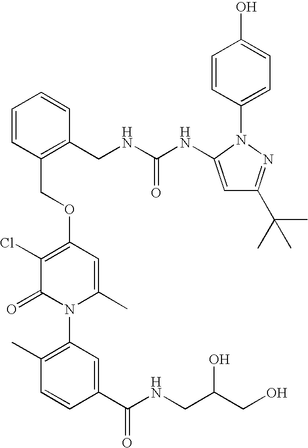

### 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-N-(2,3-dihydroxy-propyl)-4-methyl-benzamide

This compound was synthesized according to General Procedure J from 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.30 g, 0.448 mmol), 3-amino1,2-propanediol (0.0816 g, 1.79 mmol), and EDCI (0.515 g, 2.69 mmol) in the yield of 0.141 g (42.3%): ¹H NMR (DMSO-d₆/400 MHz) δ 9.72 (s, 1H), 8.44-8.42 (m, 1H), 8.10 (s, 1H), 7.86-7.84 (m, 1H), 7.60 (s, 1H), 7.53-7.52 (m, 2H), 7.38-7.20 (m, 5H), 7.05-7.04 (m, 1H), 6.85-6.83 (m, 2H), 6.73 (s, 1H), 6.22 (s, 1H), 5.41 (s, 2H), 4.83-4.79 (m, 1H), 4.56-4.53 (m, 1H), 4.38 (d, 2H, J=5.6 Hz), 3.65-3.10 (m, 5H), 2.00 (s, 3H), 1.89 (s, 3H), 1.23 (s, 9H); Anal. Calcd for C₃₉H₄₃ClN₆O₇-¾H2O: C, 61.90; H, 5.93; N, 11.10. Found: C, 61.50; H, 5.83; N, 10.57; ES-MS m/z 743.18, 745.13 (C₃₉H₄₃ClN₆O₇ requires 743.26).

### Example 151

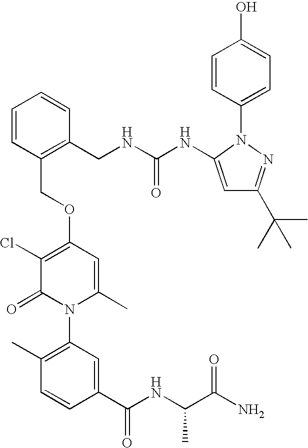

### (R)-3-[4-(2-{3-[5-tert-Butyl-2-(4-hydroxy-phenyl)-2H-pyrazol-3-yl]-ureidomethyl}-benzyloxy)-3-chloro-6-methyl-2-oxo-2H-pyridin-1-yl]-N-(1-carbamoyl-ethyl)-4-methyl-benzamide

This compound was synthesized according to General Procedure J from 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.30 g, 0.448 mmol), L-alaninamide which were prepared from L-alaninamide hydrochloride (0.223 g, 1.79 mmol), sodium hydride (60%, 0.054 g, 1.34 mmol) and EDCI (0.515 g, 2.69 mmol) in the yield of 0.155 g (46.7%): ¹H NMR (DMSO-d₆/400 MHz) δ 9.72 (s, 1H), 8.44-8.42 (m, 1H), 8.10 (s, 1H), 7.86-7.84 (m, 1H), 7.60 (s, 1H), 7.53-7.52 (m, 2H), 7.38-7.20 (m, 5H), 7.05-7.04 (m, 1H), 6.85-6.83 (m, 2H), 6.73 (s, 1H), 6.22 (s, 1H), 5.42 (s, 2H), 4.43-4.37 (m, 3H), 2.00 (s, 3H), 1.89 (s, 3H), 1.31 (d, 3H, J=6.8 Hz), 1.23 (s, 9H); Anal. Calcd for C₃₉H₄₂ClN₇O₆—H₂O: C, 61.78; H, 5.85; N, 12.93. Found: C, 61.90; H, 5.99; N, 11.99; ES-MS m/z 740.10, 742.11 (C₃₉H₄₂ClN₇O₆ requires 740.25).

### Example 152

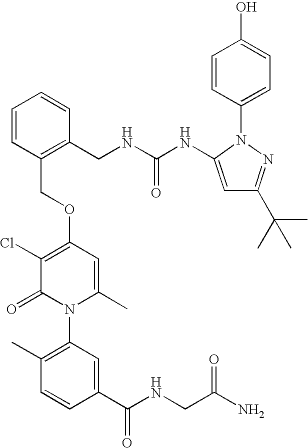

### 3-[4-(2-{3-[5-tert-Butyl-2-(4-hydroxy-phenyl)-2H-pyrazol-3-yl]-ureidomethyl}-benzyloxy)-3-chloro-6-methyl-2-oxo-2H-pyridin-1-yl]-N-carbamoylmethyl-4-methyl-benzamide

This compound was synthesized according to General Procedure J from 3-[4-(2-{3-[5-tert-butyl-2-(4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.30 g, 0.448 mmol), glycinamide which was prepared from glycinamide hydrochloride (0.198 g, 1.79 mmol), sodium hydride (60%, 0.054 g, 1.34 mmol) and EDCI (0.515 g, 2.69 mmol) in the yield of 0.121 g (37.2%): ¹H NMR (DMSO-d₆/400 MHz) δ 9.72 (s, 1H), 8.44-8.42 (m, 1H), 8.10 (s, 1H), 7.86-7.84 (m, 1H), 7.60 (s, 1H), 7.53-7.52 (m, 2H), 7.38-7.20 (m, 5H), 7.05-7.04 (m, 1H), 6.85-6.83 (m, 2H), 6.73 (s, 1H), 6.22 (s, 1H), 5.42 (s, 2H), 4.38 (d, 2H, J=5.6 Hz), 3.82-3.77 (m, 2H), 2.02 (s, 3H), 1.92 (s, 3H), 1.23 (s, 9H); Anal. Calcd for C₃₈H₄₀ClN₇O₆-½EtOAc: C, 62.37; H, 5.78; N, 12.73. Found: C, 61.99; H, 5.77; N, 12.68; ES-MS m/z 726.15, 728.16 (C₃₈H₄₀ClN₇O₆ requires 726.23).

### Example 153

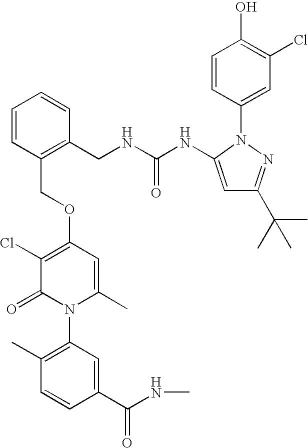

### 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4,N-dimethyl-benzamide

This compound was synthesized according to General Procedure J from 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), methylamine (2M in THF, 0.7 mL, 1.419 mmol) and EDCI (0.408 g, 2.13 mmol) in the yield of 0.079 g (31.0%): M.p. 201-203° C.; ¹H NMR (DMSO-d₆/400 MHz) δ 10.50 (s, 1H), 8.44-8.44 (m, 1H), 8.21 (s, 1H), 7.87-7.85 (m, 1H), 7.60 (s, 1H), 7.52-7.50 (m, 2H), 7.42-7.22 (m, 5H), 7.05-6.99 (m, 2H), 6.74 (s, 1H), 6.23 (s, 1H), 5.41 (s, 2H), 4.38 (d, 2H, J=5.6 Hz), 2.77 (d, 2H, J=3.4 Hz), 2.00 (s, 3H), 1.89 (s, 3H), 1.23 (s, 9H); Anal. Calcd for C₃₇H₃₈Cl₂N₆O₅-½EtOAc: C, 61.49; H, 5.55; N, 11.03. Found: C, 61.07; H, 5.59; N, 11.13; ES-MS m/z 717.01, 719.25 (C₃₇H₃₈Cl₂N₆O₅ requires 717.65).

### Example 154

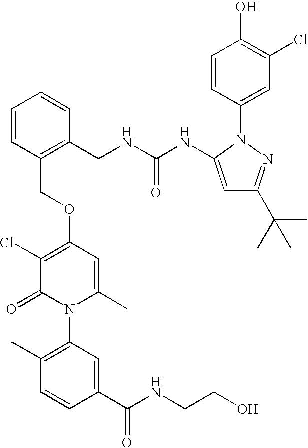

### 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-N-(2-hydroxy-ethyl)-4-methyl-benzamide

This compound was synthesized according to General Procedure J from 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), ethanolamine (0.043 g, 0.710 mmol) and EDCI (0.408 g, 2.13 mmol) in the yield of 0.092 g (34.7%): M.p. 195-196° C.; ¹H NMR (DMSO-d₆/400 MHz) δ 10.50 (s, 1H), 8.44-8.44 (m, 1H), 8.21 (s, 1H), 7.87-7.85 (m, 1H), 7.60 (s, 1H), 7.52-7.50 (m, 2H), 7.42-7.22 (m, 5H), 7.05-6.99 (m, 2H), 6.74 (s, 1H), 6.23 (s, 1H), 5.41 (s, 2H), 4.74 (br, 1H), 4.38 (d, 2H, J=5.6 Hz), 3.51-3.28 (m, 4H), 2.00 (s, 3H), 1.89 (s, 3H), 1.23 (s, 9H); Anal. Calcd for C₃₈H₄₀Cl₂N₆O₆-½EtOAc: C, 60.68; H, 5.60; N, 10.61. Found: C, 60.59; H, 5.66; N, 10.63; ES-MS m/z 747.19, 749.11 (C₃₈H₄₀Cl₂N₆O₆ requires 747.68).

### Example 155

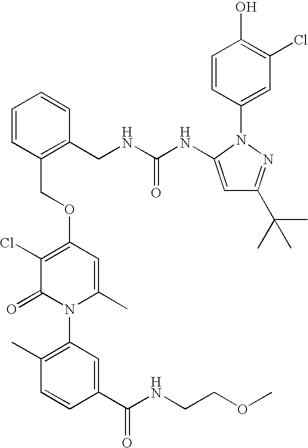

### 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-N-(2-methoxy-ethyl)-4-methyl-benzamide

This compound was synthesized according to General Procedure J from 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), 2-methoxyethanolamine (0.106 g, 1.42 mmol) and EDCI (0.408 g, 2.13 mmol) in the yield of 0.091 g (33.7%): M.p. 179-180° C.; ¹HNMR (DMSO-d₆/400 MHz) δ 10.50 (s, 1H), 8.44-8.44 (m, 1H), 8.21 (s, 1H), 7.87-7.85 (m, 1H), 7.60 (s, 1H), 7.52-7.50 (m, 2H), 7.42-7.22 (m, 5H), 7.05-6.99 (m, 2H), 6.74 (s, 1H), 6.23 (s, 1H), 5.41 (s, 2H), 4.38 (d, 2H, J=5.6 Hz), 3.51-3.28 (m, 7H), 2.00 (s, 3H), 1.89 (s, 3H), 1.23 (s, 9H); Anal. Calcd for C₃₉H₄₂Cl₂N₆O₆-¼EtOAc: C, 61.30; H, 5.66; N, 10.72. Found: C, 61.35; H, 5.76; N, 10.57; ES-MS m/z 761.22, 763.22 (C₃₉H₄₂Cl₂N₆O₆ requires 761.70).

### Example 156

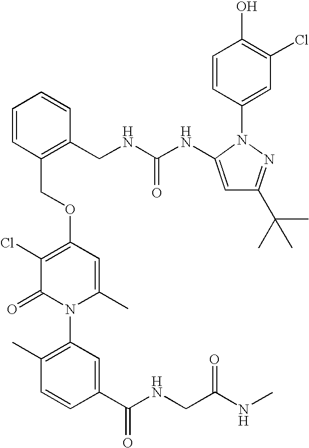

### 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-N-methylcarbamoylmethyl-benzamide

This compound was synthesized according to General Procedure J from 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.30 g, 0.417 mmol), 2-amino-N-methyl-acetamide (0.147 g, 1.67 mmol) and EDCI (0.480 g, 2.50 mmol) in the yield of 0.102 g (31.6%): M.p. 209-211° C.; ¹H NMR (DMSO-d₆/400 MHz) δ 10.50 (s, 1H), 8.44-8.44 (m, 1H), 8.21 (s, 1H), 7.87-7.85 (m, 1H), 7.60 (s, 1H), 7.52-7.50 (m, 2H), 7.42-7.22 (m, 5H), 7.05-6.99 (m, 2H), 6.74 (s, 1H), 6.23 (s, 1H), 5.41 (s, 2H), 4.38 (d, 2H, J=5.6 Hz), 3.84-3.77 (m, 2H), 2.60 (d, 3H, J=4.4 Hz), 2.02 (s, 3H), 1.92 (s, 3H), 1.23 (s, 9H); Anal. Calcd for C₃₉H₄₁Cl₂N₇O₆-0.8EtOAc: C, 59.97; H, 5.65; N, 11.60. Found: C, 59.39; H, 5.40; N, 12.16; ES-MS m/z 774.20, 776.29 (C₃₉H₄₁Cl₂N₇O₆ requires 774.70).

### Example 157

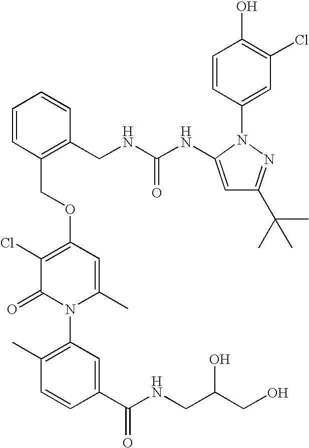

### 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-N-(2,3-dihydroxy-propyl)-4-methyl-benzamide

This compound was synthesized according to General Procedure J from 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.30 g, 0.417 mmol), 3-amino-1,2-propanediol (0.155 g, 1.70 mmol) and EDCI (0.490 g, 2.55 mmol) in the yield of 0.095 g (28.7%): M.p. 173-175° C.; ¹H NMR (DMSO-d₆/400 MHz) δ 10.50 (s, 1H), 8.44-8.44 (m, 1H), 8.21 (s, 1H), 7.87-7.85 (m, 1H), 7.60 (s, 1H), 7.52-7.50 (m, 2H), 7.42-7.22 (m, 5H), 7.05-6.99 (m, 2H), 6.74 (s, 1H), 6.23 (s, 1H), 5.41 (s, 2H), 4.84-4.80 (m, 1H), 4.57-4.55 (m, 1H), 4.38 (d, 2H, J=5.6 Hz), 4.02-3.12 (m, 5H), 2.00 (s, 3H), 1.92 (s, 3H), 1.23 (s, 9H); Anal. Calcd for C₃₉H₄₂Cl₂N₇O₆-1EtOAc: C, 59.65; H, 5.82; N, 9.71. Found: C, 59.17; H, 5.66; N, 10.11; ES-MS m/z 777.12, 779.15 (C₃₉H₄₂Cl₂N₇O₆ requires 777.70).

### Example 158

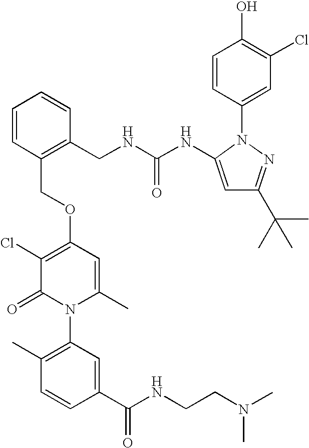

### 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-N-(2-dimethylamino-ethyl)-4-methyl-benzamide

This compound was synthesized according to General Procedure J from 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.30 g, 0.417 mmol), N,N′-dimethylethylenediamine (0.125 g, 1.419 mmol) and CDI (0.173 g, 1.064 mmol) in the yield of 0.221 g (80.4%): M.p. 181-182° C.; ¹H NMR (MeOH-d₄/400 MHz) δ 7.91-7.88 (m, 1H), 7.62 (s, 1H), 7.53-7.51 (m, 2H), 7.40-7.29 (m, 4H), 7.18-7.15 (m, 1H), 6.68 (s, 1H), 6.27 (s, 1H), 5.41 (s, 2H), 4.47 (s, 2H), 3.28 (t, 2H, J=6.4 Hz), 2.60 (t, 2H), 2.33 (s, 6H), 2.07 (s, 3H), 1.97 (s, 3H), 1.29 (s, 9H); Anal. Calcd for C₄₀H₄₅Cl₂N₇O₅-¾H₂O: C, 60.15; H, 6.02; N, 12.27. Found: C, 59.98; H, 5.97; N, 12.12; ES-MS m/z 774.22, 776.20 (C₄₀H₄₅Cl₂N₇O₅ requires 774.74).

### Example 159

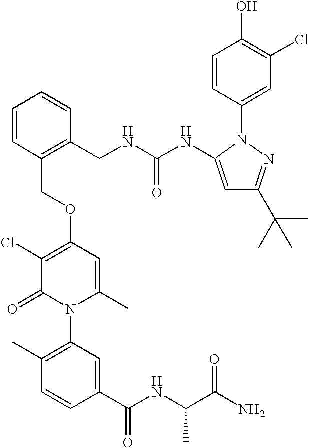

### (R)-3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-N-(1-carbamoyl-ethyl)-4-methyl-benzamide

This compound was synthesized according to General Procedure J from 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), L-alaninamide which was prepared from L-alaninamide hydrochloride (0.221 g, 1.77 mmol), sodium hydride (60%, 0.057 g, 1.42 mmol) and CDI (0.173 g, 1.064 mmol) in the yield of 0.233 g (84.8%): M.p. 192-193° C.; ¹H NMR (DMSO-d₆/400 MHz) δ 10.50 (s, 1H), 8.44-8.44 (m, 1H), 8.21 (s, 1H), 7.87-7.85 (m, 1H), 7.60 (s, 1H), 7.52-7.50 (m, 2H), 7.42-7.22 (m, 5H), 7.05-6.99 (m, 2H), 6.74 (s, 1H), 6.23 (s, 1H), 5.41 (s, 2H), 4.43-4.37 (m, 3H), 2.00 (d, 3H, J=4.3 Hz), 2.00 (s, 3H), 1.92 (s, 3H), 1.23 (s, 9H); Anal. Calcd for C₃₉H₄₁Cl₂N₇O₆-⅔EtOAc: C, 60.04; H, 5.06; N, 11.76. Found: C, 59.65; H, 5.51; N, 12.01; ES-MS m/z 774.09, 776.05 (C₃₉H₄₁Cl₂N₇O₆ requires 774.70).

### Example 160

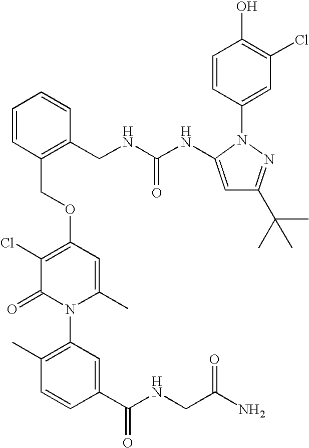

### 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-N-carbamoylmethyl-4-methyl-benzamide

This compound was synthesized according to General Procedure J from 3-[4-(2-{3-[5-tert-butyl-2-(3-chloro-4-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), glycinamide (prepared from glycinamide hydrochloride, 0.196 g, 1.77 mmol), sodium hydride (60%, 0.057 g, 1.42 mmol) and CDI (0.173 g, 1.064 mmol) in the yield of 0.202 g (74.8%): M.p. 228-230° C.; ¹H NMR (DMSO-d₆/400 MHz) δ 10.50 (s, 1H), 8.44-8.44 (m, 1H), 8.21 (s, 1H), 7.87-7.85 (m, 1H), 7.60 (s, 1H), 7.52-7.50 (m, 2H), 7.42-7.22 (m, 5H), 7.05-6.99 (m, 2H), 6.74 (s, 1H), 6.23 (s, 1H), 5.41 (s, 2H), 4.38 (d, 2H, J=5.6 Hz), 3.82-3.76 (m, 2H), 2.00 (s, 3H), 1.92 (s, 3H), 1.23 (s, 9H); Anal. Calcd for C₃₈H₃₉Cl₂N₇O₆: C, 60.00; H, 5.17; N, 12.89. Found: C, 60.02; H, 5.08; N, 12.78; ES-MS m/z 760.10, 762.08 (C₃₈H₃₉Cl₂N₇O₆ requires 760.67).

### Example 161

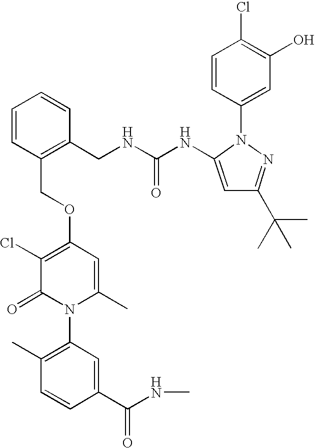

### 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4,N-dimethyl-benzamide

This compound was synthesized according to General Procedure J from 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.30 g, 0.426 mmol), methylamine (2M in THF, 0.85 mL, 1.70 mmol) and EDCI (0.449 g, 2.56 mmol) in the yield of 0.100 g (32.5%): M.p. 201-203° C.; ¹H NMR (DMSO-d₆/400 MHz) δ 10.56 (s, 1H), 8.44-8.42 (m, 1H), 8.30 (s, 1H), 7.85-7.82 (m, 1H), 7.59 (s, 1H), 7.51-7.48 (m, 2H), 7.40-7.27 (m, 4H), 7.11-6.89 (m, 3H), 6.72 (s, 1H), 6.24 (s, 1H), 5.39 (s, 2H), 4.36 (d, 2H, J=5.6 Hz), 2.77 (d, 2H, J=3.4 Hz), 2.00 (s, 3H), 1.89 (s, 3H), 1.23 (s, 9H); Anal. Calcd for C₃₇H₃₈Cl₂N₆O₅-⅔EtOAc: C, 61.37; H, 5.62; N, 10.82. Found: C, 60.94; H, 5.55; N, 10.91; ES-MS m/z 717.21, 719.23 (C₃₇H₃₈Cl₂N₆O₅ requires 717.65).

### Example 162

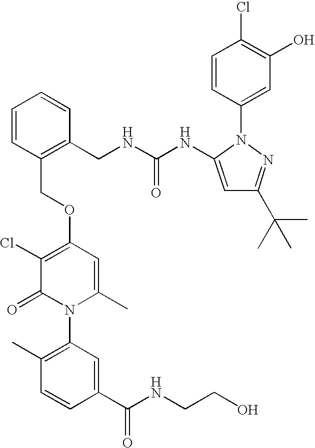

### 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-N-(2-hydroxy-ethyl)-4-methyl-benzamide

This compound was synthesized according to General Procedure J from 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), ethanolamine (0.087 g, 1.42 mmol) and EDCI (0.408 g, 2.13 mmol) in the yield of 0.075 g (28.3%): M.p. 196-197° C.; ¹H NMR (DMSO-d₆/400 MHz) δ 10.56 (s, 1H), 8.44-8.42 (m, 1H), 8.30 (s, 1H), 7.85-7.82 (m, 1H), 7.59 (s, 1H), 7.51-7.48 (m, 2H), 7.40-7.27 (m, 4H), 7.11-6.89 (m, 3H), 6.72 (s, 1H), 6.24 (s, 1H), 5.39 (s, 2H), 4.74 (t, 1H, J=5.6 Hz), 4.38 (d, 2H, J=5.6 Hz), 3.47-3.27 (m, 4H), 2.00 (s, 3H), 1.89 (s, 3H), 1.23 (s, 9H); Anal. Calcd for C₃₈H₄₀Cl₂N₆O₆-½EtOAc: C, 60.68; H, 5.60; N, 10.61. Found: C, 60.17; H, 5.71; N, 10.52; ES-MS m/z 747.13, 749.07 (C₃₈H₄₀Cl₂N₆O₆ requires 747.68).

### Example 163

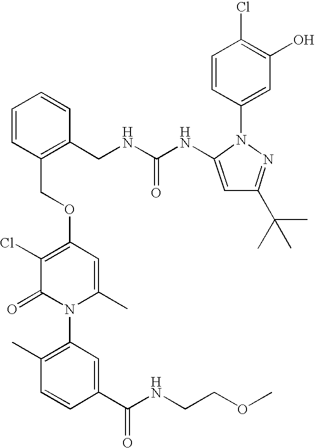

### 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-N-(2-methoxy-ethyl)-4-methyl-benzamide

This compound was synthesized according to General Procedure J from 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), 2-methoxyethanolamine (0.106 g, 1.42 mmol) and EDCI (0.408 g, 2.13 mmol) in the yield of 0.079 g (29.3%): M.p. 182-183° C.; ¹H NMR (DMSO-d₆/400 MHz) δ 10.54 (s, 1H), 8.44-8.42 (m, 1H), 8.30 (s, 1H), 7.85-7.82 (m, 1H), 7.59 (s, 1H), 7.51-7.48 (m, 2H), 7.40-7.27 (m, 4H), 7.11-6.89 (m, 3H), 6.72 (s, 1H), 6.24 (s, 1H), 5.39 (s, 2H), 4.38 (d, 2H, J=5.6 Hz), 3.45-3.26 (m, 7H), 2.00 (s, 3H), 1.89 (s, 3H), 1.23 (s, 9H); Anal. Calcd for C₃₉H₄₂Cl₂N₆O₆-EtOAc: C, 61.50; H, 5.93; N, 9.89. Found: C, 60.09; H, 5.69; N, 10.16; ES-MS m/z 761.12, 763.03 (C₃₉H₄₂Cl₂N₆O₆ requires 761.70).

### Example 164

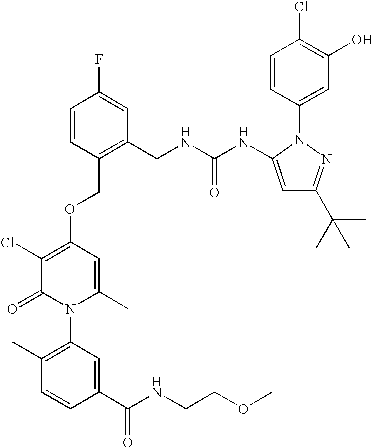

### 3-[4-(2-{3-[5-tert-butyl-2-(4-chloro-3-hydroxy-phenyl)-2H-pyrazol-3-yl]-ureidomethyl}-4-fluoro-benzyloxy)-3-chloro-6-methyl-2-oxo-2H-pyridin-1-yl]-N-(2-methoxy-ethyl)-4-methyl-benzamide

This compound was synthesized according to General Procedure J from 3-[4-(2-{3-[5-tert-butyl-2-(4-chloro-3-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid (1.228 g, 1.7 mmol), 2-methoxyethanolamine (0.511 g, 6.8 mmol) and CDI (0.827 g, 5.1 mmol) in the yield of 1.1 g (83.0%): M.p. 186-187° C.; ¹H NMR (CD₃OD/400 MHz) δ 7.87 (m, 1H), 7.60-7.50 (m, 3H), 7.35 (m, 1H), 7.10-7.04 (m, 3H), 6.90 (m, 1H), 6.67 (s, 1H), 6.29 (s, 1H), 5.35 (s, 2H), 4.46 (s, 2H), 3.54 (s, 2H), 5.35 (s, 3H), 3.30 (s, 2H), 2.09 (s, 3H); 1.98 (s, 3H); 1.29 (s, 9H); Anal. Calcd for C₃₉H₄₁Cl₂FN₆O₆-⅓H₂O: C, 59.62; H, 5.35; N, 10.70. Found: C, 59.60; H, 5.31; N, 10.36; ES-MS m/z 781.05, 779.10 (C₃₉H₄₁Cl₂FN₆O₆ requires 779.70).

### Example 165

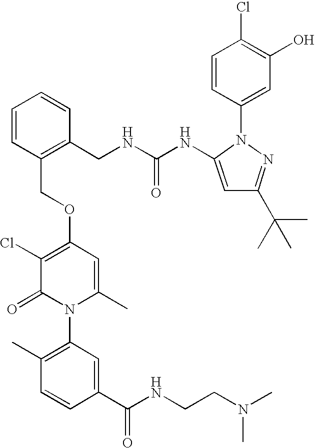

### 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-N-(2-dimethylamino-ethyl)-4-methyl-benzamide

This compound was synthesized according to General Procedure J from 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), N,N′-dimethylethylenediamine (0.125 g, 1.419 mmol) and CDI (0.173 g, 1.064 mmol) in the yield of 0.176 g (64.1%): M.p. 185-186° C.; ¹H NMR (MeOH-d₄/400 MHz) δ 7.91-7.88 (m, 1H), 7.62 (s, 1H), 7.53-7.51 (m, 2H), 7.37-7.30 (m, 3H), 7.02-7.01 (m, 1H), 6.86-6.84 (m, 1H), 6.65 (s, 1H), 6.29 (s, 1H), 5.39 (s, 2H), 4.47 (s, 2H), 3.53 (t, 2H, J=6.4 Hz), 2.60 (t, 2H), 2.34 (s, 6H), 2.07 (s, 3H), 1.97 (s, 3H), 1.29 (s, 9H); Anal. Calcd for C₄₀H₄₅Cl₂N₇O₅-1H₂O: C, 60.60; H, 5.98; N, 12.37. Found: C, 60.29; H, 5.97; N, 12.21; ES-MS m/z 774.16, 776.12 (C₄₀H₄₅Cl₂N₇O₅ requires 774.74).

### Example 166

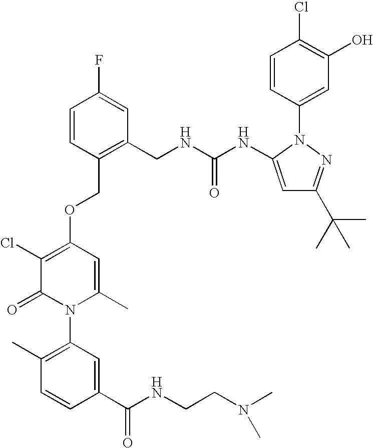

### 3-[4-(2-{3-[5-tert-Butyl-2-(4-chloro-3-hydroxy-phenyl)-2H-pyrazol-3-yl]-ureidomethyl}-4-fluoro-benzyloxy)-3-chloro-6-methyl-2-oxo-2H-pyridin-1-yl]-N-(2-dimethylamino-ethyl)-4-methyl-benzamide

This compound was synthesized according to General Procedure J from 3-[4-(2-{3-[5-tert-butyl-2-(4-chloro-3-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid (0.25 g, 0.346 mmol), N,N′-dimethylethylenediamine (0.122 g, 1.384 mmol) and CDI (0.168 g, 1.038 mmol) in the yield of 0.247 g (90.0%): M.p. 177-178° C.; ¹H NMR (MeOH-d₄/400 MHz) δ 7.90-7.87 (m, 1H), 7.63 (s, 1H), 7.56-7.51 (m, 2H), 7.35-7.32 (m, 1H); 7.10-7.02 (m, 3H), 6.88-6.86 (m, 1H), 6.66 (s, 1H), 6.29 (s, 1H), 5.35 (s, 2H), 4.46 (s, 2H), 3.51 (t, 2H, J=6.4 Hz), 2.63 (t, 2H, J=6.4 Hz), 2.36 (s, 6H), 2.07 (s, 3H), 1.98 (s, 3H), 1.29 (s, 9H); Anal. Calcd for C₄₀H₄₄FCl₂N₇O₅-1H₂O: C, 59.26; H, 5.72; N, 11.96. Found: C, 59.31; H, 5.48; N, 11.75; ES-MS m/z 794.13, 792.14 (C₄₀H₄₄FCl₂N₇O₅ requires 792.74).

### Example 167

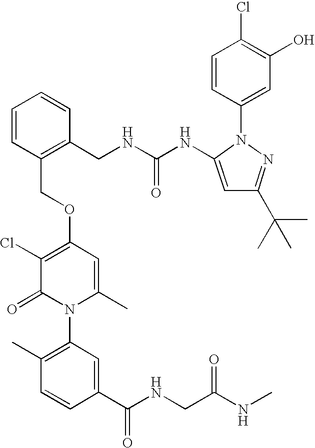

### 3-[4-(2-{3-[5-tert-Butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-N-methylcarbamoylmethyl-benzamide

This compound was synthesized according to General Procedure J from 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), 2-amino-N-methyl-acetamide (0.125 g, 1.42 mmol) and CDI (0.173 g, 1.064 mmol) in the yield of 0.149 g (54.2%): M.p. 179-181° C.; ¹H NMR (DMSO-d₆/400 MHz) δ 10.55 (s, 1H), 8.44-8.42 (m, 1H), 8.30 (s, 1H), 7.85-7.82 (m, 1H), 7.59 (s, 1H), 7.51-7.48 (m, 2H), 7.40-7.27 (m, 4H), 7.11-6.89 (m, 3H), 6.72 (s, 1H), 6.24 (s, 1H), 5.41 (s, 2H), 4.38 (d, 2H, J=5.6 Hz), 3.84-3.77 (m, 2H), 2.60 (d, 3H, J=4.4 Hz), 2.02 (s, 3H), 1.92 (s, 3H), 1.23 (s, 9H); Anal. Calcd for C₃₉H₄₁Cl₂N₇O₆-⅔H₂O: C, 59.54; H, 5.42; N, 12.46. Found: C, 59.35; H, 5.46; N, 12.83; ES-MS m/z 774.16, 776.17 (C₃₉H₄₁Cl₂N₇O₆ requires 774.70).

### Example 168

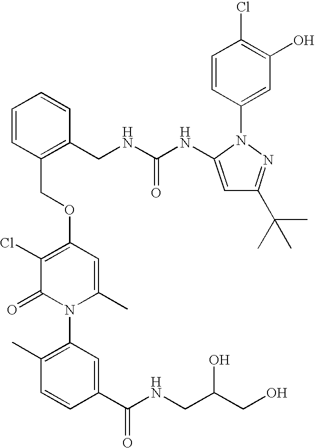

### 3-[4-(2-{3-[5-tert-Butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-N-(2,3-dihydroxy-propyl)-4-methyl-benzamide

This compound was synthesized according to General Procedure J from 3-[4-(2-{3-[5-tert-butyl-2-(4-chloro-3-hydroxy-phenyl)-2H-pyrazol-3-yl]-ureidomethyl}-benzyloxy)-3-chloro-6-methyl-2-oxo-2H-pyridin-1-yl]-4-methyl-benzoic acid (0.25 g, 0.355 mmol), 3-amino-1,2-propanediol (0.129 g, 1.42 mmol) and CDI (0.173 g, 1.06 mmol) in the yield of 0.210 g (76.1%): M.p. 169-171° C.; ¹H NMR (DMSO-d₆/400 MHz) δ 10.55 (s, 1H), 8.44-8.42 (m, 1H), 8.30 (s, 1H), 7.85-7.82 (m, 1H), 7.59 (s, 1H), 7.51-7.48 (m, 2H), 7.40-7.27 (m, 4H), 7.11-6.89 (m, 3H), 6.72 (s, 1H), 6.24 (s, 1H), 5.41 (s, 2H), 4.84-4.80 (m, 1H), 4.57-4.55 (m, 1H), 4.38 (d, 2H, J=5.6 Hz), 4.02-3.12 (m, 5H), 2.00 (s, 3H), 1.92 (s, 3), 1.23 (s, 9H); Anal. Calcd for C₃₉H₄₂Cl₂N₇O₆-¾H₂O: C, 58.43; H, 5.62; N, 10.48. Found: C, 58.27; H, 5.58; N, 10.32; ES-MS m/z 777.11, 779.08 (C₃₉H₄₂Cl₂N₇O₆ requires 777.70).

### Example 169

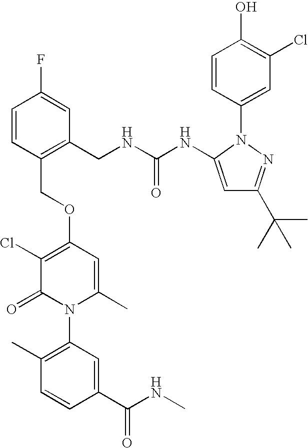

### 3-[4-(2-{3-[5-tert-butyl-2-(3-chloro-4-hydroxy-phenyl)-2H-pyrazol-3-yl]-ureidomethyl}-4-fluoro-benzyloxy)-3-chloro-6-methyl-2-oxo-2H-pyridin-1-yl]-4,N-dimethyl-benzamide

This compound was synthesized according to General Procedure J from 3-[4-(2-{3-[5-tert-butyl-2-(3-chloro-4-hydroxy-phenyl)-2H-pyrazol-3-yl]-ureidomethyl}-4-fluoro-benzyloxy)-3-chloro-6-methyl-2-oxo-2H-pyridin-1-yl]-4-methyl-benzoic acid (1.6 g, 2.22 mmol), methylamine (2 M in THF, 2.22 mL, 4.44 mmol), and CDI (1.40 g, 8.85 mmol) in the yield of 0.75 g (46.3%): %); M.p. 177-180° C. ¹H NMR (DMSO-d₆/400 MHz) δ 10.42 (s, 1H), 8.42 (bs, 1H), 8.22 (bs, 1H), 7.9 (m, 1H), 7.7-7.00 (m, 7H), 6.22 (s, 1H), 6.75 (s, 1H), 5.3 (s, 2H), 4.28 (m, 2H), 2.8 (m, 3H), 2.01 (s, 3H), 1.95 (s, 3H), 1.21 (s, 9H), Anal. Calcd for C₃₇H₃₇Cl₂FN₆O₅-¼CH₂Cl₂: C, 59.11; H, 4.99; N, 11.10. Found: C, 59.30; H, 5.17; N, 11.01; ES-MS m/z 735.06, 737.06 (C₃₇H₃₇Cl₂FN₆O₅ requires 735.65).

### General Procedure K

To a solution of 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-4-methyl-benzoic acid methyl ester in ethanol was added lithium hydroxide (10 equiv.) in water (1M) at 0° C. The mixture was stirred at room temperature overnight, concentrated and the residue was neutralized with 1M citric acid to pH 1. The solid separated was filtered and dried to give the desired product, which can be used in the proceeding steps without further purification.

### Example 170

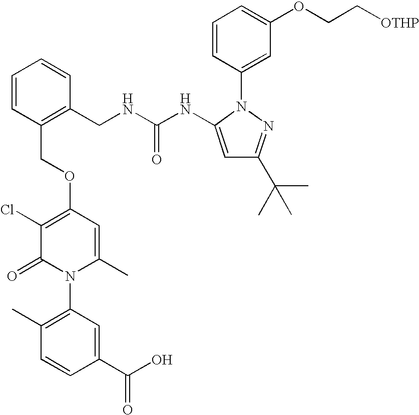

### 3-(4-[(2-{3-(5-tert-butyl-2-[(3-[2-(tetrahydro-pyran-2-yloxy)-ethoxy]-phenyl]-2H-pyrazol-3-yl)-ureidomethyl]-benzyloxy}-3-chloro-6-methyl-2-oxo-2H-pyridin-1-yl)-4-methyl-benzoic acid

This compound was synthesized according to General Procedure K from 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-4-methyl-benzoic acid methyl ester (0.30 g, 0.369 mmol) in the yield of 0.295 g (100%): ¹H NMR (DMSO-d₆/400 MHz) δ 13.11 (br, 1H), 8.32 (s, 1H), 7.96-7.94 (m, 1H), 7.71 (s, 1H), 7.57-7.51 (m, 2H), 7.40-7.31 (m, 4H), 7.08-7.06 (m, 3H), 6.98-6.96 (m, 1H), 6.73 (s, 1H), 6.27 (s, 1H), 5.40 (s, 2H), 4.65 (br, 1H), 4.38 (d, 2H, J=5.6 Hz), 4.16-4.14 (m, 2H), 4.00-3.90 (m, 1H), 3.77-3.70 (m, 2H), 3.44-3.41 (m, 1H), 2.02 (s, 3H), 1.89 (s, 3H), 1.72-1.58 (m, 2H), 1.52-1.38 (m, 4H), 1.25 (s, 9H); ES-MS m/z 798.14, 801.08 (C₄₃H₄₈ClN₅O₈ requires 798.33).

### General Procedure L

To the solution of 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-4-methyl-benzoic acid in DMF (4-10 mL) was added CDI (1.2 equiv) at 0° C. under nitrogen. The mixture was stirred at room temperature for 30 min and then the amine derivative was added to the reaction mixture at 0° C. The mixture was allowed to warm up to room temperature while stirring overnight then concentrated from most of the DMF and the residue was treated with ice-water (5 mL). The white solid separated was collected by filtration and purified by silica gel flash chromatography using dichloromethane/methanol (10:1) as eluant to give the desired product.

### Example 171

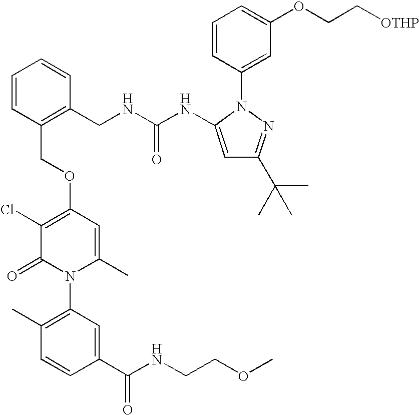

### 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-N-(2-methoxy-ethyl)-4-methyl-benzamide

This compound was synthesized according to General Procedure L from 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-4-methyl-benzoic acid (0.30 g, 0.376 mmol), 2-methoxyethanolamine (0.056 g, 0.752 mmol) and CDI (0.073 g, 0.451 mmol) in the yield of 0.301 g (93.6%): ¹H NMR (DMSO-d₆/400 MHz) δ 8.54 (s, 1H), 8.31 (m, 1H), 7.89-7.87 (m, 1H), 7.65 (s, 1H), 7.53-7.51 (m, 2H), 7.40-7.31 (m, 4H), 7.08-7.06 (m, 3H), 6.98-6.96 (m, 1H), 6.74 (s, 1H), 6.27 (s, 1H), 5.39 (s, 2H), 4.65 (br, 1H), 4.38 (d, 2H, J=5.6 Hz), 4.16-4.14 (m, 2H), 4.00-3.90 (m, 1H), 3.77-3.70 (m, 2H), 3.45-3.26 (m, 8H), 2.00 (s, 3H), 1.89 (s, 3H), 1.72-1.58 (m, 2H), 1.52-1.38 (m, 4H), 1.23 (s, 9H); ES-MS m/z 855.22, 857.23 (C₄₆H₅₅ClN₆O₈ requires 855.43).

### Example 172

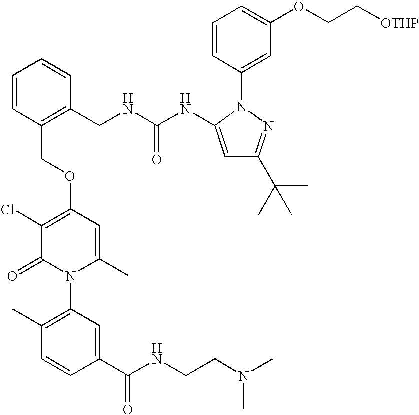

### 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-N-(2-dimethylamino-ethyl)-4-methyl-benzamide

This compound was synthesized according to General Procedure J from 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-4-methyl-benzoic acid (0.330 g, 0.413 mmol), N,N′-dimethylethylenediamine (0.073 g, 0.827 mmol) and CDI (0.080 g, 0.496 mmol) in the yield of 0.159 g (54.3%): ¹H NMR (DMSO-d₄/400 MHz) δ 8.40 (s, 1H), 8.31 (s, 1H), 7.89-7.87 (m, 1H), 7.65 (s, 1H), 7.53-7.51 (m, 2H), 7.40-7.31 (m, 4H), 7.08-7.06 (m, 3H), 6.98-6.96 (m, 1H), 6.74 (s, 1H), 6.27 (s, 1H), 5.39 (s, 2H), 4.65 (br, 1H), 4.38 (d, 2H, J=5.6 Hz), 4.16-4.14 (m, 2H), 4.00-3.90 (m, 1H), 3.77-3.70 (m, 2H), 3.45-3.26 (m, 3H), 2.41-2.39 (m, 2H), 2.18 (s, 6H), 2.00 (s, 3), 1.89 (s, 3H), 1.72-1.58 (m, 2H), 1.52-1.38 (m, 4H), 1.23 (s, 9H); ES-MS m/z 868.16, 870.15 (C₄₇H₅₈ClN₇O₇ requires 868.47).

### General Procedure M

To the solution of 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-N-(2-methoxy-ethyl)-4-methyl-benzamide in methanol was added pyridinium p-toluenesulfonate (0.3 eqiv) and the mixture was stirred at 50° C. for 6 hours. The reaction mixture was concentrated under reduced pressure and the residue was purified by silica gel flash chromatography using ethyl acetate-methanol (10:1) as eluant to give the title compound as a white powder.

### Example 173

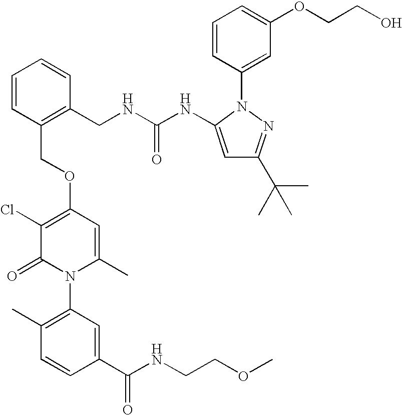

### 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid 2-methoxy-ethyl ester

This compound was synthesized according to General Procedure M from 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-N-(2-methoxy-ethyl)-4-methyl-benzamide (0.246 g, 0.288 mmol) in the yield of 0.183 g (82.5%): M.p. 143-145° C.; ¹H NMR (DMSO-d₆/400 MHz) δ 8.53 (s, 1H), 8.31 (m, 1H), 7.89-7.87 (m, 1H), 7.65 (s, 1H), 7.53-7.51 (m, 2H), 7.40-7.31 (m, 4H), 7.08-7.06 (m, 3H), 6.98-6.96 (m, 1H), 6.74 (s, 1H), 6.27 (s, 1H), 5.39 (s, 2H), 4.89 (br, 1H), 4.39 (d, 2H, J=4.4 Hz), 4.03-4.00 (m, 2H), 3.72-3.70 (m, 2H), 3.45-3.32 (m, 4H), 3.26 (s, 3H), 2.00 (s, 3H), 1.93 (s, 3H), 1.25 (s, 9H); Anal. Calcd for C₄₁H₄₇ClN₆O₇: C, 63.58; H, 6.14; N, 10.90. Found: C, 63.46; H, 6.30; N, 10.64; ES-MS m/z 771.14, 773.09 (C₄₁H₄₇ClN₆O₇ requires 771.31).

### Example 174

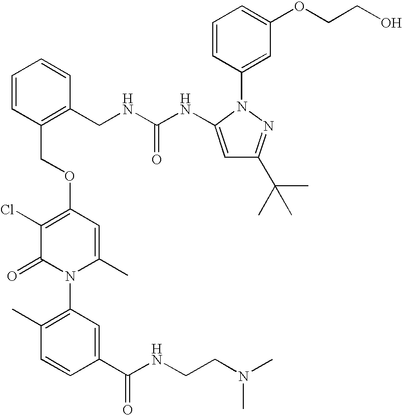

### 3-{4-[2-(3-{5-tert-butyl-2-[3-(2-hydroxy-ethoxy)-phenyl]-2H-pyrazol-3-yl}-ureidomethyl)-benzyloxy]-3-chloro-6-methyl-2-oxo-2H-pyridin-1-yl}-4-methyl-benzoic acid 2-dimethylamino-ethyl ester

This compound was synthesized according to General Procedure M from 3-(4-{2-[3-(5-tert-butyl-2-{3-[2-(tetrahydro-pyran-2-yloxy)-ethoxy]-phenyl}-2H-pyrazol-3-yl)-ureidomethyl]-benzyloxy}-3-chloro-6-methyl-2-oxo-2H-pyridin-1-yl)-N-(2-dimethylamino-ethyl)-4-methyl-benzamide (0.183 g, 0.288 mmol) in the yield of 0.123 g (74.5%): M.p. 164-165° C.; ¹H NMR (MeOH-d₆/400 MHz) δ 7.90-7.88 (m, 1H), 7.62 (s, 1H), 7.53-7.51 (m, 2H), 7.38-7.31 (m, 4H), 7.05-7.00 (m, 3H), 6.67 (s, 1H), 6.31 (s, 1H), 5.39 (s, 2H), 4.47 (s, 2H), 4.05-4.03 (m, 2H), 3.86-3.84 (m, 2H), 3.52 (t, 2H, J=6.4 Hz), 2.59 (t, 2H), 2.33 (s, 6H), 2.07 (s, 3H), 1.97 (s, 3H), 1.30 (s, 9H); Anal. Calcd for C₄₂H₅₀ClN₇O₆-½H₂O: C, 63.59; H, 6.48; N, 12.36. Found: C, 63.33; H, 6.35; N, 12.09; ES-MS m/z 784.16, 786.16 (C₄₂H₅₀ClN₇O₆ requires 784.35).

### Example 175

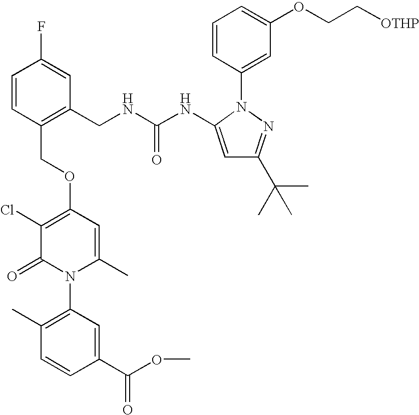

### 3-(4-{2-[3-(5-tert-butyl-2-{3-[2-(tetrahydro-pyran-2-yloxy)-ethoxy]-phenyl}-2H-pyrazol-3-yl)-ureidomethyl]-4-fluoro-benzyloxy}-3-chloro-6-methyl-2-oxo-2H-pyridin-1-yl)-4-methyl-benzoic acid methyl ester

Phosgene (20% solution in toluene, 5.20 mL) was added to a mixture of 5-tert-butyl-2-{3-[2-(tetrahydro-pyran-2-yloxy)-ethoxy]-phenyl}-2H-pyrazol-3-ylamine (1.778 g, 4.96 mmol), dichloromethane (86 mL) and saturated solution of NaHCO₃ (94 mL) at 0° C. After 15 min. most of volatiles were removed under vacuum and the residue was dissolved in 5 mL of THF. To the solution was added a solution of 3-[4-(2-aminomethyl-4-fluoro-benzyloxy)-3-chloro-6-methyl-2-oxo-2H-pyridin-1-yl]-4-methyl-benzoic acid methyl ester (2.0 g, 4.496 mmol) in THF (20 mL) at 0° C. and the resulting mixture was stirred at room temperature overnight (18 hr). The reaction mixture was concentrated under reduced pressure and the residue was purified by silica gel flash chromatography using ethyl acetate as eluat to give the title compound (3.026 g, 81.1%). ¹H NMR (DMSO-d₆/400 MHz) δ 8.38 (s, 1H), 7.99-7.96 (m, 1H), 7.76 (s, 1H), 7.60-6.56 (m, 2H), 7.39-7.35 (m, 1H), 7.19-7.09 (m, 5H), 6.97-6.95 (m, 1H), 6.74 (s, 1H), 6.28 (s, 1H), 5.37 (s, 2H), 4.64 (s, 1H), 4.40 (d, 2H, J=5.2 Hz), 4.16 (s, 2H), 3.93-3.70 (m, 6H), 3.44-3.41 (m, 1H), 2.03 (s, 3H), 1.89 (s, 3H), 1.70-1.61 (m, 2H), 1.47-1.44 (m, 4H), 1.30 (s, 9H).

### Example 176

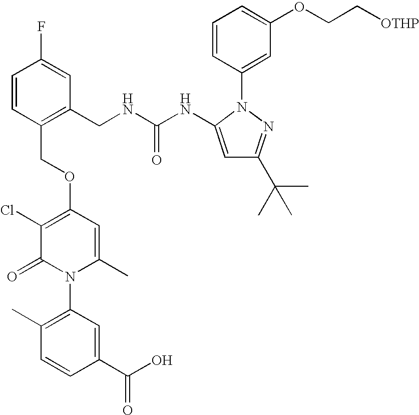

### 3-(4-{2-[3-(5-tert-butyl-2-{3-[2-(tetrahydro-pyran-2-yloxy)-ethoxy]-phenyl}-2H-pyrazol-3-yl)-ureidomethyl]-4-fluoro-benzyloxy}-3-chloro-6-methyl-2-oxo-2H-pyridin-1-yl)-4-methyl-benzoic acid

This compound was synthesized according to General Procedure K from 3-(4-{2-[3-(5-tert-butyl-2-{3-[2-(tetrahydro-pyran-2-yloxy)-ethoxy]-phenyl}-2H-pyrazol-3-yl)-ureidomethyl]-4-fluoro-benzyloxy}-3-chloro-6-methyl-2-oxo-2H-pyridin-1-yl)-4-methyl-benzoic acid methyl ester (2.902 g, 3.495 mmol) in the yield of 2.733 g (95.8%): ¹H NMR (DMSO-d₆/400 MHz) δ 8.38 (s, 1H), 7.99-7.96 (m, 1H), 7.76 (s, 1H), 7.60-6.56 (m, 2H), 7.39-7.35 (m, 1H), 7.19-7.09 (m, 5H), 6.97-6.95 (m, 1H), 6.74 (s, 1H), 6.28 (s, 1H), 5.37 (s, 2H), 4.64 (s, 1H), 4.40 (d, 2H, J=5.2 Hz), 4.16 (s, 2H), 3.93-3.90 (m, 1H), 3.78-3.69 (m, 2H), 3.44-3.41 (m, 1), 2.03 (s, 3H), 1.89 (s, 3H), 1.70-1.61 (m, 2H), 1.47-1.44 (m, 4H), 1.30 (s, 9H).

### Example 177

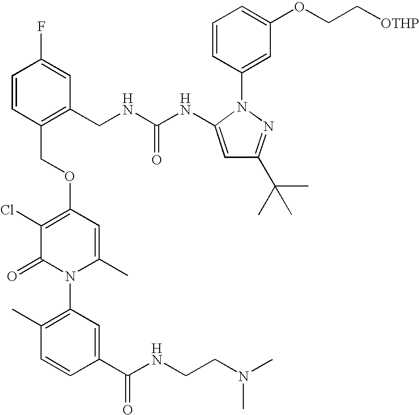

### 3-(4-{2-[3-(5-tert-butyl-2-{3-[2-(tetrahydro-pyran-2-yloxy)-ethoxy]-phenyl}-2H-pyrazol-3-yl)-ureidomethyl]-4-fluoro-benzyloxy}-3-chloro-6-methyl-2-oxo-2H-pyridin-1-yl)-N-(2-dimethylamino-ethyl)-4-methyl-benzamide

This compound was synthesized according to General Procedure D 3-(4-{2-[3-(5-tert-butyl-2-{3-[2-(tetrahydro-pyran-2-yloxy)-ethoxy]-phenyl}-2H-pyrazol-3-yl)-ureidomethyl]-4-fluoro-benzyloxy}-3-chloro-6-methyl-2-oxo-2H-pyridin-1-yl)-4-methyl-benzoic acid (0.40 g, 0.490 mmol), N,N′-dimethylethylenediamine (0.086 g, 0.98 mmol), and CDI (0.096 g, 0.588 mmol) in the yield of 0.409 g (94.2%): ¹H NMR (DMSO-d₄/400 MHz) δ 7.90-7.87 (m, 1H), 7.62 (s, 1H), 7.54-7.51 (m, 2H), 7.40-7.35 (m, 1H), 7.08-7.00 (m, 5H), 6.69 (s, 1H), 6.31 (s, 1H), 5.37 (s, 2H), 4.68 (br, 1H), 4.46 (s, 2H), 4.16-4.14 (m, 2H), 4.99-3.73 (m, 3H), 3.52-3.50 (m, 3H), 2.58-2.55 (m, 2H), 2.31 (s, 6H), 2.08 (s, 3H), 1.99 (s, 3H), 1.72-1.58 (m, 2H), 1.52-1.38 (m, 4H), 1.23 (s, 9H); ES-MS m/z 886.27, 888.21 (C₄₇H₅₇ClFN₇O₇ requires 886.46).

### Example 178

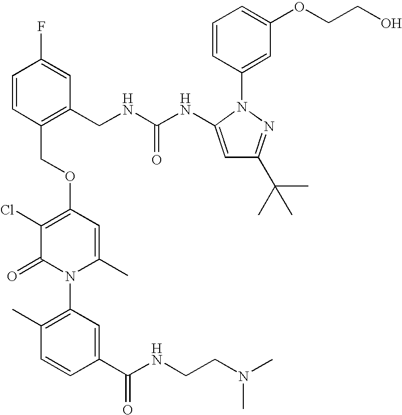

### 3-{4-[2-(3-{5-tert-butyl-2-[3-(2-hydroxy-ethoxy)-phenyl]-2H-pyrazol-3-yl}-ureidomethyl)-4-fluoro-benzyloxy]-3-chloro-6-methyl-2-oxo-2H-pyridin-1-yl}-4-methyl-benzoic acid 2-dimethylamino-ethyl ester

This compound was synthesized according to General Procedure M from 3-(4-{2-[3-(5-tert-butyl-2-{3-[2-(tetrahydro-pyran-2-yloxy)-ethoxy]-phenyl}-2H-pyrazol-3-yl)-ureidomethyl]-4-fluoro-benzyloxy}-3-chloro-6-methyl-2-oxo-2H-pyridin-1-yl)-N-(2-dimethylamino-ethyl)-4-methyl-benzamide (0.400 g, 0.451 mmol) in the yield of 0.224 g (67.4%): M.p. 158-160° C.; ¹H NMR (MeOH-d₆/400 MHz) δ 7.90-7.88 (m, 1H), 7.62 (s, 1H), 7.53-7.51 (m, 2H), 7.38-7.35 (m, 1H), 7.07-7.00 (m, 5H), 6.68 (s, 1H), 6.31 (s, 1H), 5.36 (s, 2H), 4.46 (s, 2H), 4.05 (t, 2H, J=4.8 Hz), 3.85 (t, 2H), 3.52 (t, 2H, J=6.8 Hz), 2.58 (t, 2H), 2.32 (s, 6H), 2.07 (s, 3H), 1.98 (s, 3H), 1.30 (s, 9H); Anal. Calcd for C₄₂H₄₉ClFN₇O₆: C, 62.87; H, 6.16; N, 12.22. Found: C, 62.88; H, 6.14; N, 11.93; ES-MS m/z 802.19, 804.13 (C₄₂H₄₉ClFN₇O₆ requires 802.34).

### Example 179

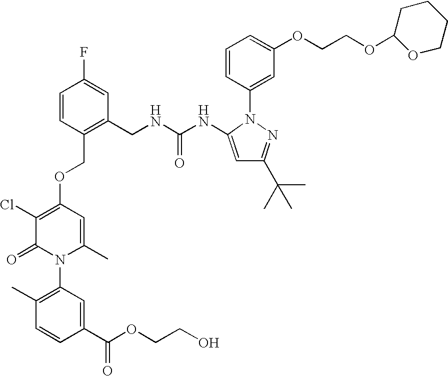

### 3-(4-{2-[3-(5-tert-butyl-2-{3-[2-(tetrahydro-pyran-2-yloxy)-ethoxy]-phenyl}-2H-pyrazol-3-yl)-ureidomethyl]-4-fluoro-benzyloxy}-3-chloro-6-methyl-2-oxo-2H-pyridin-1-yl)-4-methyl-benzoic acid 2-hydroxy-ethyl ester

This compound was synthesized according to General Procedure J 3-(4-{2-[3-(5-tert-butyl-2-{3-[2-(tetrahydro-pyran-2-yloxy)-ethoxy]-phenyl}-2H-pyrazol-3-yl)-ureidomethyl]-4-fluoro-benzyloxy}-3-chloro-6-methyl-2-oxo-2H-pyridin-1-yl)-4-methyl-benzoic acid (0.49 g, 0.60 mmol), ethanolamine (0.147 g, 2.4 mmol) and CDI (0.292 g, 1.8 mmol) in the yield of 0.441 g (85.5%): ¹H NMR (CD₃OD/400 MHz) δ 7.90-7.88 (m, 1H), 7.61 (s, 1H), 7.53-7.51 (m, 2H), 7.39-7.35 (m, 1H), 7.07-7.00 (m, 5H), 6.68 (s, 1H), 6.30 (s, 1H), 5.36 (s, 2H), 4.68 (br, 1H), 4.46 (s, 2H), 4.42-3.48 (m, 10H), 2.07 (s, 3H), 1.98 (s, 3H), 1.78-1.52 (m, 6H), 1.23 (s, 9H); ES-MS m/z 860.13, 859.19 (C₄₅H₅₂ClFN₆O₈ requires 859.40).

### Example 180

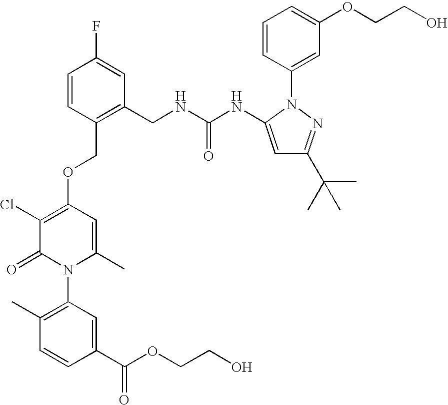

### 3-{4-[2-(3-{5-tert-butyl-2-[3-(2-hydroxy-ethoxy)-phenyl]-2H-pyrazol-3-yl}-ureidomethyl)-4-fluoro-benzyloxy]-3-chloro-6-methyl-2-oxo-2H-pyridin-1-yl}-4-methyl-benzoic acid 2-hydroxy-ethyl ester

This compound was synthesized according to General Procedure M from 3-(4-{2-[3-(5-tert-butyl-2-{3-[2-(tetrahydro-pyran-2-yloxy)-ethoxy]-phenyl}-2H-pyrazol-3-yl)-ureidomethyl]-4-fluoro-benzyloxy}-3-chloro-6-methyl-2-oxo-2H-pyridin-1-yl)-4-methyl-benzoic acid 2-hydroxy-ethyl ester (0.420 g, 0.488 mmol) in the yield of 0.30 g (79.4%): M.p. 180-181° C.; ¹H NMR (MeOH-d₆/400 MHz) δ 7.89-7.87 (m, 1H), 7.61 (s, 1 μl), 7.54-7.50 (m, 2H), 7.38-7.35 (m, 1H), 7.06-6.99 (m, 5H), 6.68 (s, 1H), 6.31 (s, 1H), 5.35 (s, 2H), 4.45 (s, 2H), 4.05-4.03 (m, 2H,), 3.85-3.83 (m, 2H), 3.69-3.66 (m, 2H,), 3.48-3.46 (m, 2H), 2.06 (s, 3H), 1.97 (s, 3H), 1.30 (s, 9H); Anal. Calcd for C₄₀H₄₄ClFN₆O₇: C, 61.97; H, 5.72; N, 10.84. Found: C, 61.68; H, 5.78; N, 10.49; ES-MS m/z 777.04, 775.05 (C₄₀H₄₄ClFN₆O₇ requires 775.28).

### Example 181

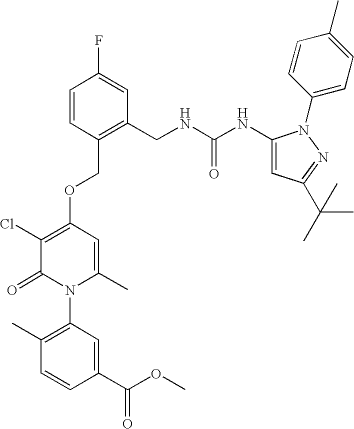

### Methyl 3-{4-[(2-{[({[3-tert-butyl-1-(4-methylphenyl)-1H-pyrazol-5-yl]amino}carbonyl)amino]methyl}-4-fluorobenzyl)oxy]-3-chloro-6-methyl-2-oxopyridin-1(2H)-yl}-4-methylbenzoate

### Step 1: Synthesis of methyl 3-(4-(2-cyano-4-fluorobenzyloxy)-3-chloro-6-methyl-2-oxopyridin-1(2H)-yl)-4-methylbenzoate

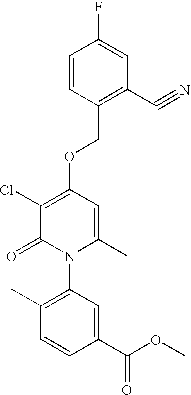

Methyl 3-(3-chloro-4-hydroxy-6-methyl-2-oxopyridin-1(2H)-yl)-4-methylbenzoate (7.03 g, 22.8 mmol) was dissolved in DMF (200 mL). Potassium carbonate (3.45 g, 25.1 mmol) and 2-(bromomethyl)-5-fluorobenzonitrile (5.37 g, 25.1 mmol) were added. The reaction was stirred at 60° C. for three hours. It was cooled to room temperature and ethyl acetate (500 mL) was added. The solution was extracted with H₂O (300 mL) and brine (300 mL). The organic phase was dried over MgSO₄, filtered, and evaporated. The resulting tan solid was recrystallized from ethyl acetate/hexane. (7.7 g, 77%) ¹HNMR (400 MHz, DMSO-d6) δ ppm 1.89 (s, 3H) 2.01 (s, 3H) 3.82 (s, 3H) 5.43 (s, 2H) 6.74 (s, 1H) 7.56 (d, J=8.06 Hz, 1H) 7.68 (m, 1H) 7.75 (d, J=1.88 Hz, 1H), 7.83 (dd. J=8.59, 5.37, 1H) 7.95 (m, 2H) HRMS (m/z) 441.0975. M+H, C₂₃H₁₈ClFN₂O₄ requires 441.1012.

### Step 2: Synthesis of Methyl 3-{4-[(2-{[({[3-tert-butyl-1-(4-methylphenyl)-1H-pyrazol-5-yl]amino}carbonyl)amino]methyl}-4-fluorobenzyl)oxy]-3-chloro-6-methyl-2-oxopyridin-1(2H)-yl}-4-methylbenzoate

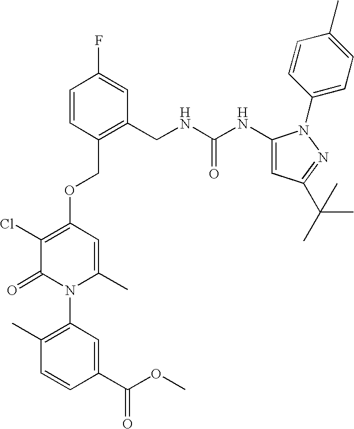

Methyl 3-(4-(2-cyano-4-fluorobenzyloxy)-3-chloro-6-methyl-2-oxopyridin-1(2H)-yl)-4-methylbenzoate (6.6 g, 15 mmol) was dissolved in THF (250 mL) and cooled to 0° C. in an ice-water bath. Borane dimethyl sulfide complex (15 mL, 30 mmol, 2.0 M in THF) was added dropwise via syringe. The reaction was stirred overnight at room temperature. Analysis by LCMS, showed incomplete reaction, so the reaction mixture was cooled to 0° C. and an additional 9 mL of the borane dimethyl sulfide complex was added. The reaction was stirred overnight at room temperature. It was still not complete, so the reaction was again cooled to 0° C. and 6 mL of the borane dimethyl sulfide complex was added. The reaction was stirred overnight at room temperature. I was cooled to 0° C. and quenched via careful addition of methanol. The mixture was evaporated. The resulting residue was dissolved in ethyl acetate (300 mL). It was extracted with H₂O (200 mL) and brine (200 mL). The organic phase was dried over MgSO₄, filtered, and evaporated. Methyl 3-(4-(2-(aminomethyl)-4-fluorobenzyloxy)-3-chloro-6-methyl-2-oxopyridin-1(2H)-yl)-4-methylbenzoate was obtained as a pale yellow solid. 3-tert-butyl-1-p-tolyl-1H-pyrazol-5-amine (1.85 g, 8.05 mmol) was dissolved in CH₂Cl₂ (150 mL). Sodium bicarbonate (aq.) (75 mL) was added, followed by phosgene (8.8 mL, 7.9 g, 16 mmol, 20 wt. % in toluene). The mixture was vigorously stirred for 20 minutes. The layers were separated and the organic phase was dried over MgSO₄, filtered, and evaporated. It was dissolved in THF (100 mL). Methyl 3-(4-(2-(aminomethyl)-4-fluorobenzyloxy)-3-chloro-6-methyl-2-oxopyridin-1(2H)-yl)-4-methylbenzoate (3.58 g, 8.05 mmol) in THF (50 mL) was added. The reaction mixture stirred at room temperature for two hours. The solvent was evaporated and the crude reaction mixture was purified by flash column chromatography. (1.6 g, 28%) ¹HNMR (400 MHz, DMSO-d6) δ ppm 1.21 (s, 9H) 1.86 (s, 3H) 2.00 (s, 3H) 2.32 (s, 3H) 3.82 (s, 3H) 4.35 (d, J=5.91 Hz, 2H) 5.34 (s, 2H) 6.22 (s, 1H) 6.71 (s, 1H) 7.05 (m, 2H) 7.14 (m, 1H) 7.24 (d, J=8.32 Hz, 2H) 7.33 (m, 2H) 7.55 (m, 2H) 7.73 (d, J=1.61 Hz 1H) 7.94 (dd, J=7.92, 1.75 Hz, 1H), 8.28 (s, 1H) HRMS (m/z) 700.2711. M+H, C₃₈H₃₉ClFN₅O₅ requires 700.2697.

Using the method described above, the following three compounds were prepared.

### Example 182

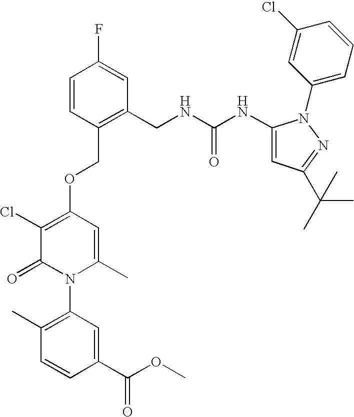

### Methyl 3-{4-[(2-{[({[3-tert-butyl-1-(3-chlorophenyl)-1H-pyrazol-5-yl]amino}carbonyl)amino]methyl}-4-fluorobenzyl)oxy]-3-chloro-6-methyl-2-oxopyridin-1(2H)-yl}-4-methylbenzoate

1HNMR (400 MHz, DMSO-d6) δ ppm 1.22 (s, 9H) 1.86 (s, 3H) 2.00 (s, 3H) 3.82 (s, 3H) 4.35 (d, J=5.64 Hz, 2H) 5.34 (s, 2H) 6.26 (s, 1H) 6.71 (s, 1H) 7.10 (m, 3H) 7.43 (m, 3H) 7.53 (m, 3H) 7.73 (d, J=1.88 Hz, 1H) 7.94 (dd, J=8.06, 1.88 Hz, 1H) 8.45 (s, 1H)

HRMS (m/z) 720.2122. M+H, C₃₇H₃₆Cl₂FN₅O₅ requires 773.2457.

### Example 183

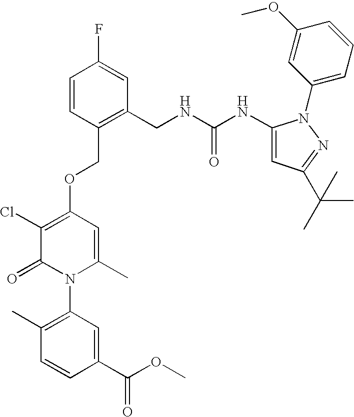

### Methyl 3-{4-[(2-{[({[3-tert-butyl-1-(3-methoxyphenyl)-1H-pyrazol-5-yl]amino}carbonyl)amino]methyl}-4-fluorobenzyl)oxy]-3-chloro-6-methyl-2-oxopyridin-1(2H)-yl}-4-methylbenzoate

1HNMR (400 MHz, DMSO-d6) δ ppm 1.22 (s, 9H) 1.86 (s, 3H) 2.00 (s, 3H) 3.74 (s, 3H) 3.82 (s, 3H) 4.36 (d, J=5.64 Hz, 2H) 5.34 (s, 2H) 6.25 (s, 1H) 6.71 (s, 1H) 6.92 (dd, J=7.25, 1.61 Hz, 1H) 7.01-7.19 (m, 5H) 7.35 (t, J=8.06 Hz, 1H) 7.48-7.60 (m, 2H) 7.73 (d, J=1.61 Hz, 1H) 7.94 (dd, J=7.92, 1.75 Hz. 1H) 8.36 (s, 1H)

HRMS (m/z) 716.2655. M+H, C₃₈H₃₉ClFN₅O₆ requires 716.2646.

### Example 184

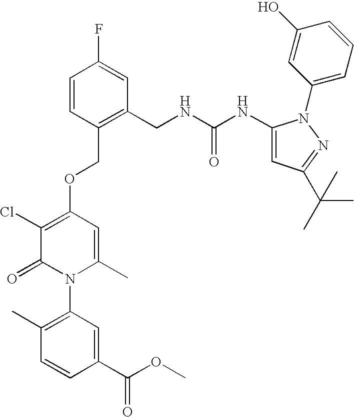

### Methyl 3-{4-[(2-{[({[3-tert-butyl-1-(3-hydroxyphenyl)-1H-pyrazol-5-yl]amino}carbonyl)amino]methyl}-4-fluorobenzyl)oxy]-3-chloro-6-methyl-2-oxopyridin-1(2H)-yl}-4-methylbenzoate

1HNMR (400 MHz, DMSO-d6) δ ppm 1.21 (s, 9H) 1.86 (s, 3H) 2.00 (s, 3H) 3.82 (s, 3H) 4.37 (d, J=5.10 Hz, 2H) 5.35 (s, 2H) 6.24 (s, 1H) 6.68-6.78 (m, 2H) 6.83-6.93 (m, 2H) 7.04-7.17 (m, 3H) 7.22 (t, J=8.19 Hz, 1H) 7.55 (t, J=7.65 Hz, 2H) 7.73 (d, J=1.34 Hz, 1H) 7.94 (dd, J=7.92, 1.48 Hz. 1H) 8.34 (s, 1H) HRMS (m/z) 702.2462. M+H, C₃₇H₃₇ClFFN₅O₅ requires 702.2495.

### Example 185

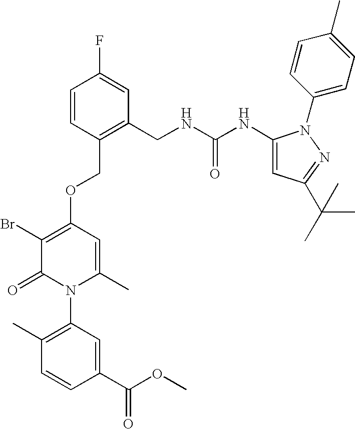

### methyl 3-{3-bromo-4-[(2-{[({[3-tert-butyl-1-(4-methylphenyl)-1H-pyrazol-5-yl]amino}carbonyl)amino]methyl}-4-fluorobenzyl)oxy]-6-methyl-2-oxopyridin-1(2H)-yl}-4-methylbenzoate

### Step 1: Synthesis of methyl 3-(4-(2-cyano-4-fluorobenzyloxy)-3-bromo-6-methyl-2-oxopyridin-1(2H)-yl)-4-methylbenzoate

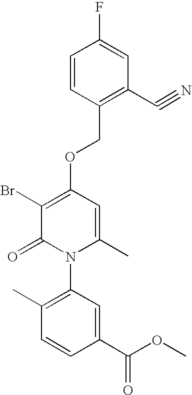

Methyl 3-(3-bromo-4-hydroxy-6-methyl-2-oxopyridin-1(2H)-yl)-4-methylbenzoate (11.21 g, 32.0 mmol) was dissolved in DMF (300 mL). Potassium carbonate (5.30 g, 38.4 mmol) and 2-(bromomethyl)-5-fluorobenzonitrile (8.21 g, 38.4 mmol) were added. The reaction was stirred at room temperature overnight. Ethyl acetate (1000 mL) was added. The solution was extracted with H₂O (500 mL) and brine (500 mL). The organic phase was dried over MgSO₄, filtered, and evaporated. The resulting tan solid was recrystallized from ethyl acetate/hexane. (12.99 g, 84%)

1HNMR (400 MHz, DMSO-d6) δ ppm 1.88 (s, 3H) 2.01 (s, 3H) 3.82 (s, 3H) 5.43 (s, 2H) 6.71 (s, 1H) 7.56 (d, J=8.06 Hz, 1H) 7.69 (td, J=8.73, 2.69 Hz, 1H) 7.75 (d, J=1.61 Hz, 1H), 7.84 (dd. J=8.73, 5.50, 1H) 7.96 (m, 2H) HRMS (m/z) 485.0506. M+H, C₂₃H₁₈BrFN₂O₄ requires 485.0507.

### Step 2: Synthesis of methyl 3-{3-bromo-4-[(2-{[({[3-tert-butyl-1-(4-methylphenyl)-1H-pyrazol-5-yl]amino}carbonyl)amino]methyl}-4-fluorobenzyl)oxy]-6-methyl-2-oxopyridin-1(2H)-yl}-4-methylbenzoate

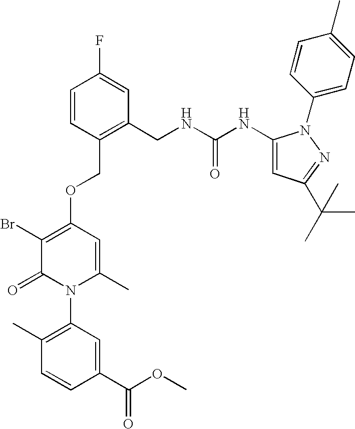

Methyl 3-(4-(2-cyano-4-fluorobenzyloxy)-3-bromo-6-methyl-2-oxopyridin-1(2H)-yl)-4-methylbenzoate (5.1 g, 10.5 mmol) was dissolved in THF (250 mL) and cooled to 0° C. in an ice-water bath. Borane dimethyl sulfide complex (10 mL, 20 mmol, 2.0 M in THF) was added dropwise via syringe. The reaction was stirred overnight at room temperature. Analysis by LCMS, showed incomplete reaction, so the reaction mixture was cooled to 0° C. and an additional 7 mL of the borane dimethyl sulfide complex was added. The reaction was stirred overnight at room temperature. It was still not complete, so the reaction was again cooled to 0° C. and 5 mL of the borane dimethyl sulfide complex was added. The reaction was stirred overnight at room temperature. I was cooled to 0° C. and quenched via careful addition of methanol. The mixture was evaporated. The resulting residue was dissolved in ethyl acetate (300 mL). It was extracted with H₂O (200 mL) and brine (200 mL). The organic phase was dried over MgSO₄, filtered, and evaporated. Methyl 3-(4-(2-(aminomethyl)-4-fluorobenzyloxy)-3-chloro-6-methyl-2-oxopyridin-1(2H)-yl)-4-methylbenzoate was obtained as a white solid.

3-tert-butyl-1-p-tolyl-1H-pyrazol-5-amine (0.94 g, 4.1 mmol) was dissolved in CH₂Cl₂ (50 mL). Sodium carbonate (aq.) (30 mL) was added, followed by phosgene (4.3 mL, 4.0 g, 8.10 mmol, 20 wt. % in toluene). The mixture was vigorously stirred for 20 minutes. The layers were separated and the organic phase was dried over MgSO₄, filtered, and evaporated. It was dissolved in THF (100 mL). Methyl 3-(4-(2-(aminomethyl)-4-fluorobenzyloxy)-3-bromo-6-methyl-2-oxopyridin-1(2H)-yl)-4-methylbenzoate (2.00 g, 4.1 mmol) in THF (30 mL) was added. The reaction mixture stirred at room temperature for two hours. The solvent was evaporated and the crude reaction mixture was purified by flash column chromatography. (0.6 g, 20%)

1HNMR (400 MHz, DMSO-d6) δ ppm 1.22 (s, 9H) 1.85 (s, 3H) 2.00 (s, 3H) 2.32 (s, 3H) 3.82 (s, 3H) 4.36 (d, J=5.64 Hz, 2H) 5.34 (s, 2H) 6.24 (s, 1H) 6.68 (s, 1H) 7.06 (m, 2H) 7.14 (td, J=8.46, 2.69 Hz, 1H) 7.25 (d, J=8.32 Hz, 2H) 7.34 (d, J=8.32 Hz, 2H) 7.55 (m, 2H) 7.72 (d, J=1.88 Hz, 1H), 7.94 (dd, J=7.92, 1.75 Hz 1H) 8.30 (s, 1H)

HRMS (m/z) 746.2155. M+H, C₃₈H₃₉BrFN₅O₅ requires 746.2179.

### Example 186

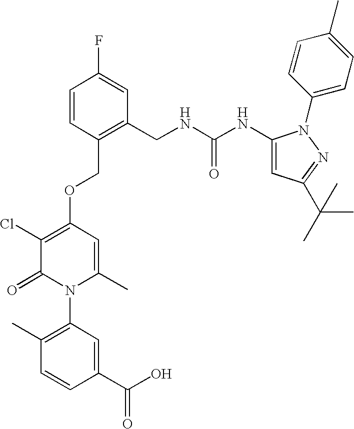

### 3-{4-[(2-{[({[3-tert-butyl-1-(4-methylphenyl)-1H-pyrazol-5-yl]amino}carbonyl)amino]methyl}-4-fluorobenzyl)oxy]-3-chloro-6-methyl-2-oxopyridin-1(2H)-yl}-4-methylbenzoic acid

Methyl 3-{4-[(2-{[({[3-tert-butyl-1-(4-methylphenyl)-1H-pyrazol-5-yl]amino}carbonyl)amino]methyl}-4-fluorobenzyl)oxy]-3-chloro-6-methyl-2-oxopyridin-1(2H)-yl}-4-methylbenzoate (1.5 g, 2.14 mmol) was dissolved in a THF/EtOH/H₂O solution (20 mL, 14 mL/4 mL/2 mL respectively). Sodium hydroxide (1 mL, 2.5 mmol, 2.5 N) was added. The reaction stirred overnight at room temperature. The reaction mixture was diluted with ethyl acetate (50 mL). The solution was extracted with 0.1 N HCl (25 mL) and brine (25 mL). The organic phase was dried over MgSO₄, filtered, and evaporated. The resulting white solid was washed with ether. (1.32 g, 90%)

1HNMR (400 MHz, DMSO-d6) δ ppm 1.22 (s, 9H) 1.86 (s, 3H) 1.99 (s, 3H) 2.32 (s, 3H) 4.35 (d, J=5.91 Hz, 2H) 5.34 (s, 2H) 6.23 (s, 1H) 6.71 (s, 1H) 7.06 (m, 2H) 7.14 (td, J=8.53, 2.82 Hz 1H) 7.24 (d, J=8.06 Hz, 2H) 7.33 (m, 2H) 7.54 (m, 2H) 7.68 (d, J=1.61 Hz 1H) 7.92 (dd, J=8.06, 1.61 Hz, 1H), 8.29 (s, 1H)

HRMS (m/z) 686.2543. M+H, C₃₇H₃₇ClFN₅O₅ requires 686.2540.

Using the method described above, the following three compounds were prepared.

### Example 187

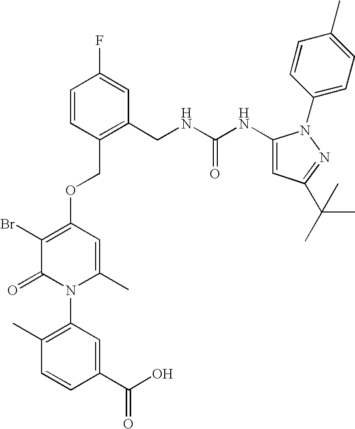

### 3-{3-bromo-4-[(2-{[({[3-tert-butyl-1-(4-methylphenyl)-1H-pyrazol-5-yl]amino}carbonyl)amino]methyl}-4-fluorobenzyl)oxy]-6-methyl-2-oxopyridin-1(2H)-yl}-4-methylbenzoic acid

1HNMR (400 MHz, DMSO-d6) δ ppm 1.22 (s, 9H) 1.85 (s, 3H) 1.99 (s, 3H) 2.32 (s, 3H) 4.35 (d, J=5.64 Hz, 2H) 5.34 (s, 2H) 6.24 (s, 1H) 6.68 (s, 1H) 7.06 (m, 2H) 7.14 (td, J=8.46, 2.69 Hz, 1H) 7.24 (d, J=8.32 Hz, 2H) 7.34 (d, J=8.32 Hz, 2H) 7.55 (m, 2H) 7.67 (d, J=1.61 Hz, 1H), 7.92 (dd, J=7.92, 1.75 Hz 1H) 8.30 (s, 1H)

HRMS (m/z) 730.2052. M+H, C₃₇H₃₇BrFN₅O₅ requires 730.2035.

### Example 188

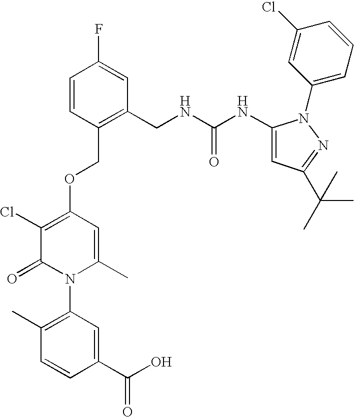

### 3-{4-[(2-{[({[3-tert-butyl-1-(3-chlorophenyl)-1H-pyrazol-5-yl]amino}carbonyl)amino]methyl}-4-fluorobenzyl)oxy]-3-chloro-6-methyl-2-oxopyridin-1(2H)-yl}-4-methylbenzoic acid

1HNMR (400 MHz, DMSO-d6) δ ppm 1.22 (s, 9H) 1.86 (s, 3H) 1.99 (s, 3H) 4.35 (d, J=5.64 Hz, 2H) 5.34 (s, 2H) 6.26 (s, 1H) 6.70 (s, 1H) 7.09 (m, 3H) 7.45 (m, 6H) 7.67 (d, J=1.07 Hz, 1H) 7.92 (dd. J=7.92, 1.48 Hz, 1H) 8.46 (s, 1H)

HRMS (m/z) 706.1960. M+H, C₃₆H₃₄Cl₂FN₅O₅ requires 706.1944.

### Example 189

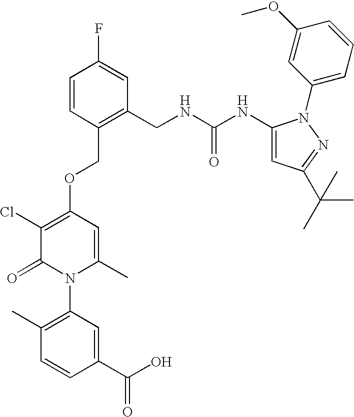

### 3-{4-[(2-{[({[3-tert-butyl-1-(3-methoxyphenyl)-1H-pyrazol-5-yl]amino}carbonyl)amino]methyl}-4-fluorobenzyl)oxy]-3-chloro-6-methyl-2-oxopyridin-1(2H)-yl}-4-methylbenzoic acid

1HNMR (400 MHz, DMSO-d6) δ ppm 1.22 (s, 9H) 1.86 (s, 3H) 1.98 (s, 3H) 3.74 (s, 3H) 4.37 (d, J=4.57 Hz, 2H) 5.34 (s, 2H) 6.32 (s, 1H) 6.69 (s, 1H) 6.91-7.00 (m, 1H) 7.02-7.20 (m, 5H) 7.37 (t, J=8.46 Hz, 1H) 7.48-7.57 (m, 2H) 7.68 (d, J=1.88 Hz, 1H) 7.92 (dd, J=7.92, 1.75 Hz. 1H) 8.47 (s, 1H) HRMS (m/z) 702.2505. M+H, C₃₇H₃₇ClFFN₅O₆ requires 702.2489.

### Example 190

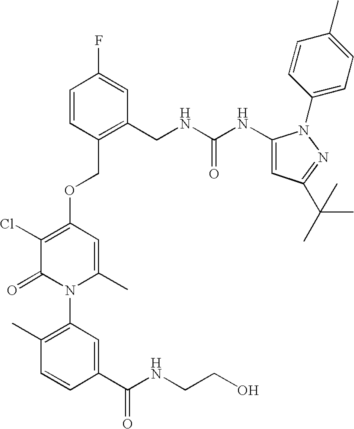

### 3-{4-[(2-{[({[3-tert-butyl-1-(4-methylphenyl)-1H-pyrazol-5-yl]amino}carbonyl)amino]methyl}-4-fluorobenzyl)oxy]-3-chloro-6-methyl-2-oxopyridin-1(2H)-yl}-N-(2-hydroxyethyl)-4-methylbenzamide

3-{4-[(2-{[({[3-tert-butyl-1-(4-methylphenyl)-1H-pyrazol-5-yl]amino)carbonyl)amino]methyl}-4-fluorobenzyl)oxy]-3-chloro-6-methyl-2-oxopyridin-1(2H)-yl}-4-methylbenzoic acid (0.114 g, 0.166 mmol) was dissolved in THF (5 mL). 2-Chloro-4,6-dimethoxy-1,3,5-triazine (0.035 g, 0.2 mmol) and N-methylmorpholine (2 drops) were added. The reaction was stirred at room temperature for four hours. Ethanolamine (0.1 mL 0.101 g, 1.66 mmol) was added. The reaction mixture was stirred overnight at room temperature. The reaction mixture was diluted with ethyl acetate (50 mL). It was extracted with H₂O (25 mL) and brine (25 mL). The organic phase was dried over MgSO₄, filtered, and evaporated. The compound was purified by flash column chromatography. A white solid was isolated. (0.080 g, 61%)

1HNMR (400 MHz, DMSO-d6) δ ppm 1.22 (s, 9H) 1.88 (s, 3H) 1.97 (s, 3H) 2.32 (s, 3H) 3.31 (m, 2H) 3.47 (t, J=6.04 Hz, 2H) 4.35 (d, J=5.64 Hz, 2H) 5.35 (s, 2H) 6.23 (s, 1H) 6.71 (s, 1H) 7.06 (dd, J=9.80, 2.82 Hz, 2H) 7.14 (m 1H) 7.25 (d, J=8.32 Hz, 2H) 7.33 (m, 2H) 7.48 (d, J=8.06 Hz, 1H) 7.54 (dd, J=8.32, 5.91 Hz, 1H) 7.62 (d, J=1.34 Hz 1H) 7.85 (dd, J=7.92, 1.75 Hz, 1H), 8.29 (s, 1H) 8.42 (t, J=5.37 Hz, 1H)

HRMS (m/z) 729.2995. M+H, C₃₉H₄₂ClFN₆O₅ requires 729.2962.

Using the method described above, the following six compounds were prepared.

### Example 191

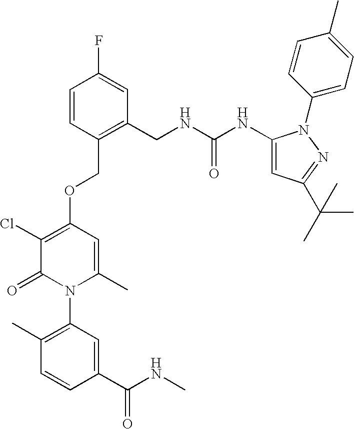

### 3-{3-chloro-4-[(2-{[({[3-tert-butyl-1-(4-methylphenyl)-1H-pyrazol-5-yl]amino}carbonyl)amino]methyl}-4-fluorobenzyl)oxy]-6-methyl-2-oxopyridin-1(2H)-yl}-N,4-dimethylbenzamide

1HNMR (400 MHz, DMSO-d6) δ ppm 1.22 (s, 9H) 1.87 (s, 3H) 1.97 (s, 3H) 2.32 (s, 3H) 2.74 (d, J=4.57 Hz, 3H) 4.35 (d, J=5.64 Hz, 2H) 5.34 (s, 2H) 6.25 (s, 1H) 6.71 (s, 1H) 7.06 (dd, J=9.67, 2.95 Hz, 2H) 7.14 (td, J=8.59, 2.69 Hz, 1H) 7.25 (d, J=8.32 Hz, 2H) 7.33 (m, 2H) 7.47 (d, J=7.79 Hz, 1H) 7.55 (m, 2H) 7.83 (dd, J=7.92, 1.75 Hz, 1H), 8.32 (s, 1H) 8.41 (d, J=4.57 Hz, 1H) HRMS (m/z) 699.2841. M+H, C₃₈H₄₀ClFN₆O₄ requires 699.2856.

### Example 192

### 3-{3-bromo-4-[(2-{[({[3-tert-butyl-1-(4-methylphenyl)-1H-pyrazol-5-yl]amino}carbonyl)amino]methyl}-4-fluorobenzyl)oxy]-6-methyl-2-oxopyridin-1(2H)-yl}-N,4-dimethylbenzamide

1HNMR (400 MHz, DMSO-d6) δ ppm 1.21 (s, 9H) 1.86 (s, 3H) 1.97 (s, 3H) 2.32 (s, 3H) 2.74 (d, J=4.30 Hz, 3H) 4.35 (d, J=5.64 Hz, 2H) 5.34 (s, 2H) 6.23 (s, 1H) 6.68 (s, 1H) 7.06 (m, 2H) 7.14 (m, 1H) 7.24 (d, J=8.32 Hz, 2H) 7.33 (m, 2H) 7.47 (d, J=8.06 Hz, 1H) 7.56 (m, 2H) 7.82 (dd, J=8.06, 1.61 Hz 1H) 8.29 (s, 1H) 8.41 (d, J=4.83 Hz, 1H) HRMS (m/z) 743.2396. M+H, C₃₈H₄₀BrFN₆O₄ requires 743.2351.

### Example 193

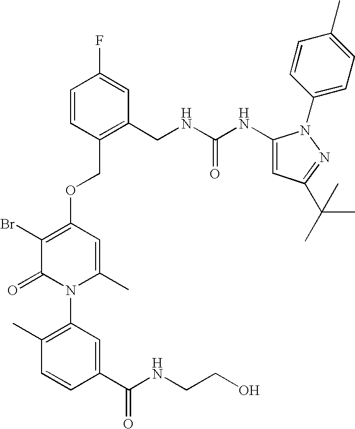

### 3-{3-bromo-4-[(2-{[({[3-tert-butyl-1-(4-methylphenyl)-1H-pyrazol-5-yl]amino}carbonyl)amino]methyl}-4-fluorobenzyl)oxy]-6-methyl-2-oxopyridin-1(2H)-yl}-N-(2-hydroxyethyl)-4-methylbenzamide

1HNMR (400 MHz, DMSO-d6) δ ppm 1.22 (s, 9H) 1.87 (s, 3H) 1.97 (s, 3H) 2.32 (s, 3H) 3.29 (m, 2H)) 3.47 (t, J=6.04 Hz, 2H) 4.36 (d, J=5.64 Hz, 2H) 5.34 (s, 2H) 6.24 (s, 1H) 6.68 (s, 1H) 7.06 (d, J=7.52 Hz, 2H) 7.14 (td, J=7.99, 2.28 Hz, 1H) 7.25 (d, J=8.06 Hz, 2H) 7.34 (d, J=8.06 Hz, 2H) 7.47 (d, J=7.79 Hz, 1H) 7.55 (m, 2H) 7.62 (s, 1H) 7.84 (d, J=8.32 Hz, 1H) 8.31 (s, 1H) 8.42 (d, J=4.83 Hz, 1H)

HRMS (m/z) 773.2477. M+H, C₃₉H₄₂BrFN₆O₅ requires 773.2457.

### Example 194

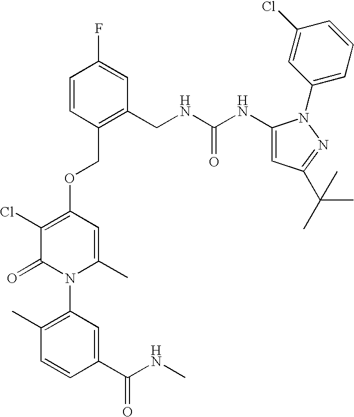

### 3-{4-[(2-{[({[3-tert-butyl-1-(3-chlorophenyl)-1H-pyrazol-5-yl]amino}carbonyl)amino]methyl}-4-fluorobenzyl)oxy]-3-chloro-6-methyl-2-oxopyridin-1(2H)-yl}-N,4-dimethylbenzamide

1HNMR (400 MHz, DMSO-d6) δ ppm 1.22 (s, 9H) 1.87 (s, 3H) 1.97 (s, 3H) 2.74 (d. J=4.56, 3H) 4.35 (d, J=5.91 Hz, 2H) 5.35 (s, 2H) 6.26 (s, 1H) 6.71 (s, 1H) 7.00-7.20 (m, 3H) 7.31-7.63 (m 7H) 7.83 (dd, J=8.06, 1.88 Hz. 1H) 8.36-8.47 (m, 2H)

HRMS (m/z) 719.2320. M+H, C₃₇H₃₇Cl₂FN₆O₄ requires 719.2310

### Example 195

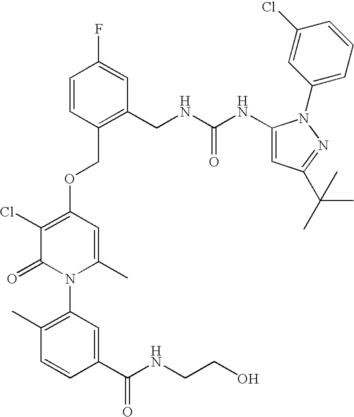

### 3-{4-[(2-{[({[3-tert-butyl-1-(3-chlorophenyl)-1H-pyrazol-5-yl]amino}carbonyl)amino]methyl}-4-fluorobenzyl)oxy]-3-chloro-6-methyl-2-oxopyridin-1(2H)-yl}-N-(2-hydroxyethyl)-4-methylbenzamide

1HNMR (400 MHz, DMSO-d6) δ ppm 1.22 (s, 9H) 1.87 (s, 3H) 1.97 (s, 3H) 3.17-3.39 (m, 2H) 3.47 (t, J=6.04 Hz, 2H) 4.35 (d, J=4.56 Hz, 2H) 5.34 (s, 2H) 6.26 (s, 1H) 6.70 (s, 1H) 6.98-7.17 (m, 3H) 7.38 (dt, J=7.52, 1.75 Hz, 1H) 7.41-7.58 (m, 5H) 7.63 (d, J=1.34 Hz, 1H) 7.85 (dd, J=7.92, 1.48 Hz. 1H) 8.37-8.44 (m, 1H) 8.48 (s, 1H). HRMS (m/z) 749.2409. M+H, C₃₈H₃₉Cl₂FN₆O₅ requires 749.2416.

### Example 196

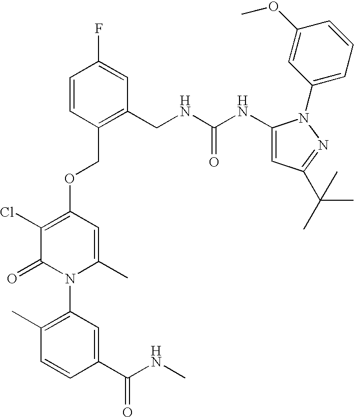

### 3-{4-[(2-{[({[3-tert-butyl-1-(3-methoxyphenyl)-1H-pyrazol-5-yl]amino}carbonyl)amino]methyl}-4-fluorobenzyl)oxy]-3-chloro-6-methyl-2-oxopyridin-1(2H)-yl}-N,4-dimethylbenzamide

1HNMR (400 MHz, DMSO-d6) δ ppm 1.22 (s, 9H) 1.87 (s, 3H) 1.97 (s, 3H) 2.74 (d, J=4.30 Hz, 3H) 3.74 (s, 3H) 4.36 (d, J=5.91 Hz, 2H) 5.35 (s, 2H) 6.25 (s, 1H) 6.71 (s, 1H) 6.93 (d, J=1.88 Hz, 1H) 6.99-7.18 (m, 5H) 7.35 (t, J=8.06 Hz, 1H) 7.48 (d, J=8.32 Hz, 2H) 7.49-7.62 (m, 2H) 7.83 (dd, J=7.92, 1.48 Hz. 1H) 8.36 (s, 1H) 8.41 (d, J=5.10 Hz, 1H) HRMS (m/z) 715.2850. M+H, C₃₈H₄₀ClFFN₆O₅ requires 715.2806.

### Example 197

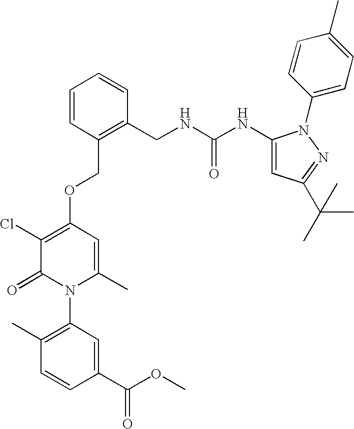

### Methyl 3-{4-[(2-{[({[3-tert-butyl-1-(4-methylphenyl)-1H-pyrazol-5-yl]amino}carbonyl)amino]methyl}benzyl)oxy]-3-chloro-6-methyl-2-oxopyridin-1(2H)-yl}-4-methylbenzoate

### Step 1. Synthesis of 2-(bromomethyl)benzaldehyde

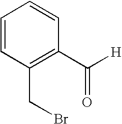

2-(Bromomethyl)benzaldehyde was prepared according to literature procedure (Xiao-Xiang Zhang and Stephen J. Lippard J. Org. Chem, 2000, 65, 5298-5305)

### Step 2: Synthesis of N-1-(2-(bromomethyl)benzyl)-3-(3-tert-butyl-1-p-tolyl-1H-pyrazol-5-yl)urea

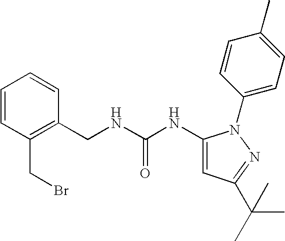

2-(Bromomethyl)benzaldehyde (0.23 g, 1.15 mmol) was dissolved in toluene (30 mL). 3-tert-Butyl-1-p-tolyl-1H-pyrazol-5-amine (0.158 g, 0.578 mmol), triethylsilane (0.37 mL, 0.269 g, 2.30 mmol), and trifluoroacetic acid (0.222 mL, 0.341 g, 2.99 mmol) were added. The reaction was stirred at 65° C. for five hours. It was allowed to cool to room temperature. Ethyl acetate (50 mL) was added and it was extracted with NaHCO₃ (aq.) (50 mL) and H₂O (50 mL). The organic phase was dried over MgSO₄, filtered, and evaporated. The crude product was purified by flash column chromatography. The resulting solid was recrystallized from ethyl acetate/hexane. (0.140 g, 53%).

1HNMR (400 MHz, DMSO-d6) δ ppm 1.22 (s, 9H) 2.33 (s, 3H) 4.35 (d, J=5.64 Hz, 2H) 4.74 (s, 2H) 6.25 (s, 1H) 6.95 (t, J=5.50 Hz, 1H) 7.15-7.35 (m, 7H) 7.34-7.46 (m, 1H) 8.21 (s, 1H) HRMS (m/z) 455.1444. M+H, C₂₃H₂₇BrN₄O requires 455.1441.

### Step 3: Synthesis of methyl 3-{4-[(2-{[({[3-tert-butyl-1-(4-methylphenyl)-1H-pyrazol-5-yl]amino}carbonyl)amino]methyl}benzyl)oxy]-3-chloro-6-methyl-2-oxopyridin-1(2H)-yl}-4-methylbenzoate

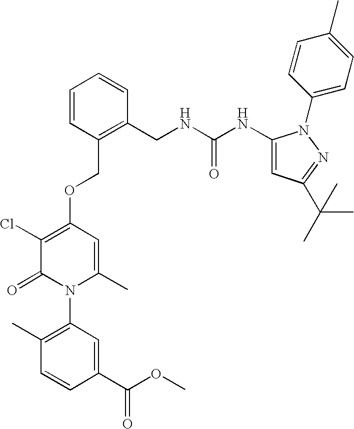

Methyl 3-(3-chloro-4-hydroxy-6-methyl-2-oxopyridin-1(2H)-yl)-4-methylbenzoate (0.067 g, 0.219 mmol) was dissolved in DMF (10 mL). N-1-(2-(bromomethyl)-benzyl)-3-(3-tert-butyl-1-p-tolyl-1H-pyrazol-5-yl)urea (0.100 g, 0.219 mmol) and potassium carbonate (0.03 g, 0.219 mmol) were added. The reaction stirred at room temperature overnight. Ethyl acetate (50 mL) was added. The solution was extracted with H₂O (30 mL) and brine (30 mL). The organic phase was dried over MgSO₄, filtered, and evaporated. The crude product was purified by flash column chromatography, and recrystallized from EtOH/H₂O. (0.075 g, 50%)

1HNMR (400 MHz, DMSO-d6) δ ppm 1.21 (s, 9H) 1.85 (s, 3H) 2.00 (s, 3H) 2.32 (s, 3H) 3.82 (s, 3H) 4.35 (d, J=5.37 Hz, 2H) 5.36 (s, 2H) 6.25 (s, 1H) 6.64-6.73 (m, 1H) 7.00 (t, J=5.77 Hz, 1H) 7.21-7.40 (m, 7H) 7.49 (dd, J=7.12, 1.21 Hz, 1H) 7.55 (d, J=8.06 Hz, 1H) 7.73 (d, J=1.88 Hz, 1H) 7.94 (dd, J=8.06, 1.61 Hz. 1H) 8.24 (s, 1H) HS (m/z) 682.2823. M+H, C₃H₄₀ClFN₅O₅ requires 682.2796.

### Example 198

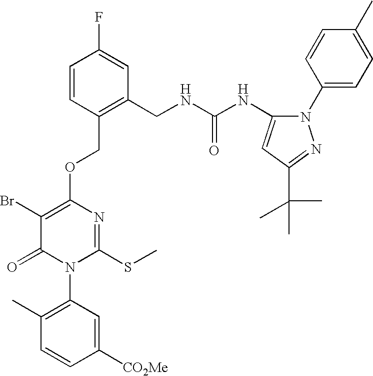

### methyl 3-{5-bromo-4-[(2-{[({[3-tert-butyl-1-(4-methylphenyl)-1H-pyrazol-5-yl]amino}carbonyl)amino]methyl}-4-fluorobenzyl)oxy]-2-(methylthio)-6-oxopyrimidin-1(6H)-yl}-4-methylbenzoate

### Step 1: Preparation of methyl 3-{4-[(2-cyano-4-fluorobenzyl)oxy]-2-(methylthio)-6-oxopyrimidin-1(6H)-yl}-4-methylbenzoate

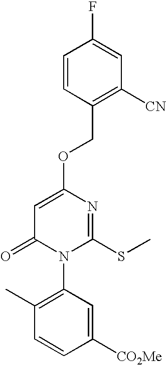

A 500 mL round bottom flask was charged with methyl 3-[4-hydroxy-2-(methylthio)-6-oxopyrimidin-1(6H)-yl]-4-methylbenzoate (WO 04/087,677 published on Oct. 14, 2004) (20 g, 65.4 mmol), potassium carbonate (10 g, 72 mmol), and N,N′-dimethylformamide (200 mL). 2-Cyano-4-fluoro-benzyl bromide (15.4 g, 72 mmol) was added and the reaction mixture was stirred under nitrogen at room temperature overnight. The reaction was quenched with water and extracted into ethyl acetate. The extract was washed with brine. The organic extract was concentrated in vacuo and the resulting residue was purified on silica, eluting with 25% ethyl acetate in hexanes and then with 100% ethyl acetate. The fractions containing product were concentrated in vacuo to give the desired product as an oil which later solidified. (23 g) ¹H NMR (400 MHz, DMSO-d₆) δ ppm 2.06-2.10 (m, 3H) 2.41 (s, 3H) 3.83 (s, 3H) 5.49 (d, J=2.42 Hz, 2H) 7.58 (d, J=8.06 Hz, 1H) 7.62-7.69 (m, 1H) 7.78 (dd, J=8.59, 5.37 Hz, 1H) 7.84 (d, J=1.88 Hz, 1H) 7.91-8.02 (m, 3H). LC/MS, tᵣ=6.67 minutes (5 to 95% acetonitrile/water over 8 minutes at 1 ml/min with detection 254 nm, at 50° C.). ES-MS m/z 440 (M+H).

### Step 2: Preparation of methyl 3-{5-bromo-4-[(2-cyano-4-fluorobenzyl)oxy]-2-(methylthio)-6-oxopyrimidin-1(6H)-yl}-4-methylbenzoate

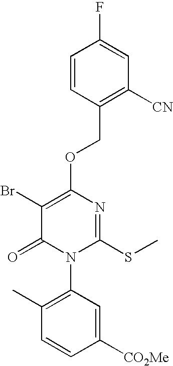

N-Bromosuccinimide (6.7 g, 37.6 mmol) was added to a 0° C. solution of methyl 3-{4-[(2-cyano-4-fluorobenzyl)oxy]-2-(methylthio)-6-oxopyrimidin-1(6H)-yl}-4-methylbenzoate (15 g, 34.2 mmol) in methylene chloride (100 mL). The reaction mixture was removed from the ice bath and was stirred at room temperature overnight. The reaction mixture was filtered and the filtrate was concentrated in vacuo. The residue was purified on silica, eluting with 1:1 hexanes:ethyl acetate. The title compound solidified after solvent was removed. (11.83 g) ¹H NMR (400 MHz, DMSO-d₆) δ ppm 2.08 (s, 3H) 2.46 (s, 3H) 3.83 (s, 3H) 5.70 (s, 2H) 7.59 (d, J=8.06 Hz, 1H) 7.63-7.70 (m, 1H) 7.76 (dd, J=8.73, 5.51 Hz, 1H) 7.85-8.11 (m, 3H). LC/MS, tᵣ=7.07 minutes (5 to 95% acetonitrile/water over 8 minutes at 1 ml/min with detection 254 nm, at 50° C.). ES-MS m/z 518/520 (M+H).

### Step 3: Preparation of methyl 3-[4-{[2-(aminomethyl)-4-fluorobenzyl]oxy}-5-bromo-2-(methylthio)-6-oxopyrimidin-1(6H)-yl]-4-methylbenzoate

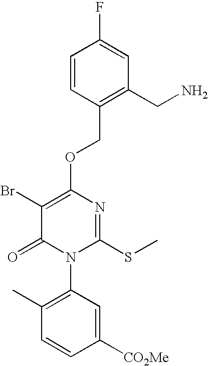

BH₃-DMS (2.0M in THF, 1.9 mmol, 3.8 mmol) was added dropwise to a 0° C. solution of methyl 3-{5-bromo-4-[(2-cyano-4-fluorobenzyl)oxy]-2-(methylthio)-6-oxopyrimidin-1(6H)-yl}-4-methylbenzoate (1 g, 1.9 mmol) in THF (50 mL). The reaction mixture was slowly warmed to room temperature overnight. The reaction mixture was cooled to 0° C. and quenched by the slow addition of 10 mL of methanol. After stirring for 15 minutes, the solution was concentrated in vacuo and the title compound was used without further purification. LC/MS, tᵣ=4.91 minutes (5 to 95% acetonitrile/water over 8 minutes at 1 ml/min with detection 254 nm, at 50° C.). ES-MS m/z 522/524 (M+H).

### Step 4: Preparation of methyl 3-{5-bromo-4-[(2-{[({[3-tert-butyl-1-(4-methylphenyl)-1H-pyrazol-5-yl]amino}carbonyl)amino]methyl}-4-fluorobenzyl)oxy]-2-(methylthio)-6-oxopyrimidin-1(6H)-yl}-4-methylbenzoate

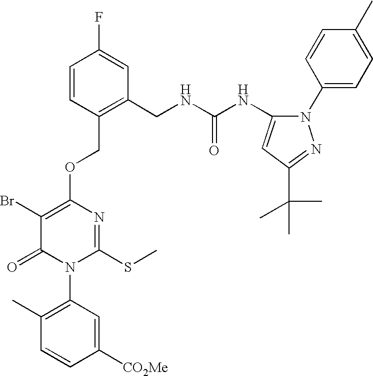

Phosgene (20% in toluene, 2 mL, 4 mmol) was added to a room temperature solution of 3-t-butyl-1-(4-methylphenyl)-1H-pyrazole-5-amine (0.45 g, 2 mmol), methylene chloride (20 mL) and saturated aqueous NaHCO₃ (20 mL). After stirring at room temperature for 15 minutes, the layers were separated and the organic layer was concentrated in vacuo. The residue was suspended in THF (20 mL) and a solution of methyl 3-[4-{[2-(aminomethyl)-4-fluorobenzyl]oxy}-5-bromo-2-(methylthio)-6-oxopyrimidin-1(6H)-yl]-4-methylbenzoate from the previous reaction (1 g, 1.9 mmol) was added. The reaction mixture was stirred under nitrogen at room temperature overnight. The reaction mixture was concentrated in vacuo. Solids were precipitated with acetonitrile/diethyl ether and discarded. The filtrate was concentrated and was purified on silica, eluting with 1:1 hexanes:ethyl acetate. The title compound was isolated as a white solid (0.399 g). ¹H NMR (400 MHz, DMSO-d₆) δ ppm 1.21 (s, 9H) 2.05 (s, 3H) 2.32 (s, 3H) 2.41-2.54 (m, 3H) 3.83 (s, 3H) 4.37 (d, J=5.64 Hz, 2H) 5.58 (d, J=7.25 Hz, 2H) 6.22 (s, 1H) 6.96-7.03 (m, 1H) 7.03-7.08 (m, 1H) 7.10-7.17 (m, 1H) 7.22-7.27 (m, 2H) 7.30-7.36 (m, 2H) 7.50 (dd, J=8.19, 6.04 Hz, 1H) 7.59 (d, J=8.06 Hz, 1H) 7.94 (d, J=1.61 Hz, 1H) 8.00 (dd, J=8.06, 1.61 Hz, 1H) 8.23 (s, 1H). LC/MS, tᵣ=7.69 minutes (5 to 95% acetonitrile/water over 8 minutes at 1 ml/min with detection 254 nm, at 50° C.). ES-MS m/z 777/779 (M+H).

### Example 199

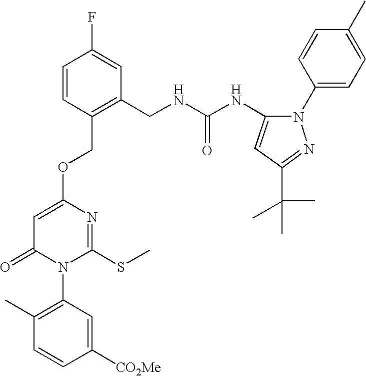

### methyl 3-{4-[(2-{[({[3-tert-butyl-1-(4-methylphenyl)-1H-pyrazol-5-yl]amino}carbonyl)amino]methyl}-4-fluorobenzyl)oxy]-2-(methylthio)-6-oxopyrimidin-1(6H)-yl}-4-methylbenzoate

### Step 1: Preparation of methyl 3-[4-{[2-(aminomethyl)-4-fluoro-benzyl]oxy}-2-(methylthio)-6-oxopyrimidin-1(6H)-yl]-4-methylbenzoate

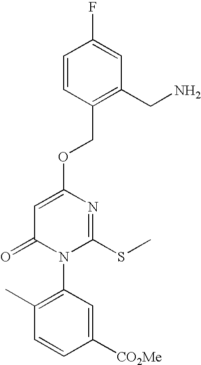

BH₃-DMS (2.0M in THF, 4.6 mL, 9.2 mmol) was added to a 0° C. solution of methyl 3-{4-[(2-cyano-4-fluorobenzyl)oxy]-2-(methylthio)-6-oxopyrimidin-1(6H)-yl}-4-methylbenzoate (2 g, 4.6 mmol) in THF (50 mL). The reaction mixture was slowly warmed to room temperature overnight. The reaction mixture was cooled to 0° C. and quenched by the slow addition of 10 mL of methanol. After stirring for 15 minutes, the solution was concentrated in vacuo and the title compound was used without further purification. (1.44 g) ¹H NMR (400 MHz, DMSO-d₆) δ ppm 2.09 (s, 3H) 2.44-2.49 (m, 3H) 3.78-3.85 (m, 3H) 5.28-5.39 (m, 2H) 5.64 (s, 1H) 7.01-7.09 (m, 1H) 7.30-7.38 (m, 1H) 7.44 (dd, J=8.46, 6.04 Hz, 1H) 7.58 (d, J=8.06 Hz, 1H) 7.83 (d, J=1.61 Hz, 1H) 7.99 (dd, J=8.06, 1.61 Hz, 1H).). LC/MS, tᵣ=6.00 minutes (5 to 95% acetonitrile/water over 8 minutes at 1 ml/min with detection 254 nm, at 50° C.). ES-MS m/z 444 (M+H).

### Step 2: Preparation of methyl 3-{4-[(2-{[({[3-tert-butyl-1-(4-methylphenyl)-1H-pyrazol-5-yl]amino}carbonyl)amino]methyl}-4-fluorobenzyl)oxy]-2-(methylthio)-6-oxopyrimidin-1(6H)-yl}-4-methylbenzoate

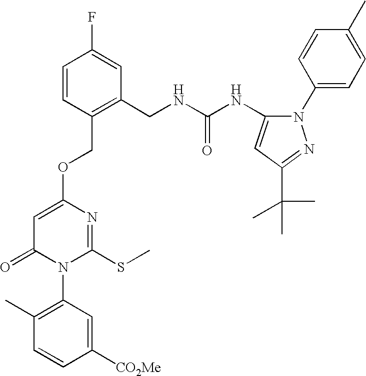

Phosgene (20% in toluene, 8.4 mL, 15.2 mmol) was added to a room temperature solution of 3-t-butyl-1-(4-methylphenyl)-1H-pyrazole-5-amine (0.87 g, 3.8 mmol), methylene chloride (20 mL) and saturated aqueous NaHCO₃ (20 mL). After stirring at room temperature for 15 minutes, the layers were separated and the organic layer was concentrated in vacuo. The residue was suspended in THF (20 mL) and a solution of methyl 3-[4-{[2-(aminomethyl)-4-fluoro-benzyl]oxy}-2-(methylthio)-6-oxopyrimidin-1(6H)-yl]-4-methylbenzoate (1.44 g, 3.3 mmol) was added. The reaction mixture was stirred under nitrogen at room temperature overnight. The reaction mixture was concentrated in vacuo. Solids were precipitated with acetonitrile/diethyl ether and discarded. The filtrate was concentrated and was purified on silica, eluting with hexanes and ethyl acetate (gradient 1:1 to 25:75 hexanes:ethyl acetate). The title compound was isolated as a white solid (1.07 g). ¹H NMR (400 MHz, DMSO-d₆) δ ppm 1.22 (s, 9H) 2.08 (s, 3H) 2.41 (s, 3H) 2.44-2.49 (m, 2H) 3.29 (s, 1H) 3.83 (s, 3H) 4.34 (d, J=5.91 Hz, 2H) 5.29-5.40 (m, 2H) 5.63 (s, 1H) 6.22 (s, 1H) 6.98 (t, J=5.77 Hz, 1H) 7.06 (dd, J=10.20, 2.69 Hz, 1H) 7.08-7.16 (m, 1H) 7.20-7.37 (m, 4H) 7.49 (dd, J=8.59, 5.91 Hz, 1H) 7.57 (d, J=8.06 Hz, 1H) 7.82 (d, J=1.61 Hz, 1H) 7.99 (dd, J=7.92, 1.75 Hz, 1H) 8.25 (s, 1H). LC/MS, tᵣ=6.21 minutes (5 to 95% acetonitrile/water over 8 minutes at 1 ml/min with detection 254 nm, at 50° C.). ES-MS m/z 699 (M+H).

### Example 200

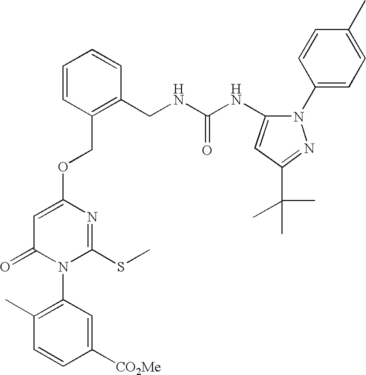

### methyl 3-{4-[(2-{[({[3-tert-butyl-1-(4-methylphenyl)-1H-pyrazol-5-yl]amino}carbonyl)amino]methyl}benzyl)oxy]-2-(methylthio)-6-oxopyrimidin-1(6H)-yl}-4-methylbenzoate

### Step 1: Preparation of 2-[2-(chloromethyl)benzyl]-1H-isoindole-1,3(2H)-dione

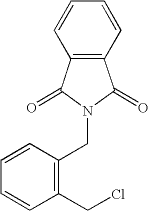

Potassium phthalimide (20 g, 108 mmol) was added to a solution of □□′-dichloroxylene (32 g, 184 mmol) in N,N-dimethyl formamide (400 mL). After stirring at room temperature under nitrogen for 18 h, the solution was filtered to remove bis-phthalimide side product. Water and ethyl acetate were added and the mixture was filtered again to remove side products. The filtrate was extracted with ethyl acetate and the organic extracts were combined and washed with brine. The organic layer was concentrated in vacuo. The resulting oil was treated with diethyl ether to precipitate the title compound as a white solid (5.98 g). ¹H NMR (400 MHz, DMSO-d₆) δ ppm 4.91 (s, 2H) 4.94 (s, 2H) 7.15-7.25 (m, 1H) 7.24-7.32 (m, 2H) 7.38-7.49 (m, 1H) 7.79-7.93 (m, 4H).

### Step 2: Preparation of methyl 3-[4-({2-[(1,3-dioxo-1,3-dihydro-2H-isoindol-2-yl)methyl]benzyl}oxy)-2-(methylthio)-6-oxopyrimidin-1(6H)-yl]-4-methylbenzoate

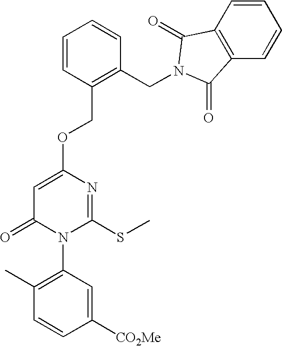

A 250 mL roundbottomed flask was charged with methyl 3-[4-hydroxy-2-(methylthio)-6-oxopyrimidin-1(6H)-yl]-4-methylbenzoate (5 g, 16.5 mmol), potassium carbonate (2.5 g, 18.1 mmol), and N,N′-dimethylformamide (100 mL). 2-[2-(Chloromethyl)benzyl]-1H-isoindole-1,3(2H)-dione (5 g, 18.1 mmol) was added and the reaction mixture was stirred under nitrogen at room temperature overnight. The reaction was quenched with water and was extracted into ethyl acetate. The extract was washed with brine. The organic extract was concentrated in vacuo and the resulting residue was purified on silica, eluting with a gradient 95:5 to 50:50 hexanes:ethyl acetate. The fractions containing product were concentrated in vacuo to give the desired product as a foam which solidified upon standing (7 g). ¹H NMR (400 MHz, DMSO-d₆) δ ppm 2.10 (s, 3H) 2.34 (s, 3H) 3.83 (s, 3H) 4.89 (s, 2H) 5.45 (q, 2H) 5.54 (s, 1H) 7.25-7.37 (m, 3H) 7.45-7.50 (m, 1H) 7.58 (d, J=8.06 Hz, 1H) 7.78-7.88 (m, 5H) 7.99 (dd, J=7.92, 1.75 Hz, 1H). LC/MS, tᵣ=3.57 minutes (5 to 95% acetonitrile/water over 5 minutes at 1 ml/min with detection 254 nm, at 50° C.). ES-MS m/z 556 (M+H).

### Step 3: Preparation of methyl 3-[4-{[2-(aminomethyl)benzyl]oxy}-2-(methylthio)-6-oxopyrimidin-1(6H)-yl]-4-methylbenzoate

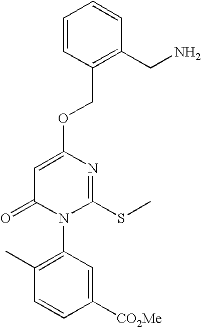

A suspension of methyl 3-[4-({2-[(1,3-dioxo-1,3-dihydro-2H-isoindol-2-yl)methyl]benzyl}oxy)-2-(methylthio)-6-oxopyrimidin-1(6H)-yl]-4-methylbenzoate (0.5 g, 0.9 mmol) in ethanol (50 mL) was heated briefly, until all of the starting material dissolved. Hydrazine hydrate (0.13 mL, (2.7 mmol) was added and the reaction mixture was stirred at room temperature overnight. The reaction mixture was concentrated in vacuo and the title compound was used without further purification. LC/MS, tᵣ=2.34 minutes (5 to 95% acetonitrile/water over 6 minutes at 1 ml/min with detection 254 nm, at 50° C.). ES-MS m/z 426 (M+H).

### Step 4: Preparation of methyl 3-{4-[(2-{[({[3-tert-butyl-1-(4-methylphenyl)-1H-pyrazol-5-yl]amino}carbonyl)amino]methyl}benzyl)oxy]-2-(methylthio)-6-oxopyrimidin-1(6H)-yl}-4-methylbenzoate

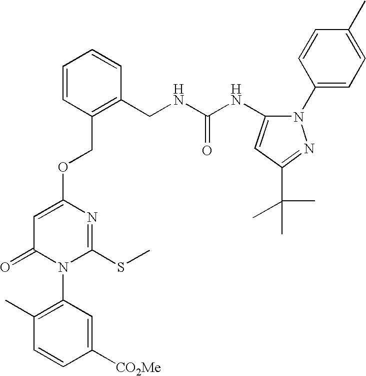

Phosgene (20% in toluene, 1 mL, 1.8 mmol) was added to a 0° C. solution of 3-t-butyl-1-(4-methylphenyl)-1H-pyrazole-5-amine (0.2 g, 0.9 mmol), methylene chloride (10 mL) and saturated aqueous NaHCO₃ (10 mL). After stirring at room temperature for 15 minutes, the layers were separated and the organic layer was concentrated in vacuo. The residue was suspended in THF (20 mL) and a solution of methyl 3-[4-{[2-(aminomethyl)benzyl]oxy}-2-(methylthio)-6-oxopyrimidin-1(6H)-yl]-4-methylbenzoate (0.9 mmol) was added. The reaction mixture was stirred under nitrogen at room temperature overnight. The reaction mixture was concentrated in vacuo. Solids were precipitated with acetonitrile/diethyl ether and discarded. The filtrate was concentrated and was purified on silica eluting with a gradient of 9:1 to 25:75 hexanes:ethyl acetate. The title compound was isolated as a white solid (75 mg). ¹H NMR (400 MHz, DMSO-d₆) δ ppm 1.21 (s, 9H) 2.05 (s, 3H) 2.32 (s, 3H) 2.40 (s, 3H) 3.83 (s, 3H) 4.33 (d, J=5.64 Hz, 2H) 5.37 (s, 2H) 5.63 (s, 1H) 6.23 (s, 1H) 6.91 (t, J=5.64 Hz, 1H) 7.18-7.38 (m, 7H) 7.44 (d, J=7.25 Hz, 1H) 7.57 (d, J=8.06 Hz, 1H) 7.82 (d, J=1.61 Hz, 1H) 7.99 (dd, J=7.92, 1.75 Hz, 1H) 8.17 (s, 1H). LC/MS, tᵣ=3.73 minutes (5 to 95% acetonitrile/water over 6 minutes at 1 ml/min with detection 254 nm, at 50° C.). ES-MS m/z 681 (M+H).

### Biological Evaluation

p38α/MK2 Cascade Assay

The ability of compounds to inhibit activated p38α was determined in a p38α/MK-2 cascade assay format. The kinase activity of p38α is determined by its ability to phosphorylate/activate nonactive MK2 (54-400). Activation of MK-2 by p38α is measured by following the phosphorylation of a MK-2 specific peptide, Hsp27 peptide (FITC-KKKALSRQLSVAA). The phosphorylation of the Hsp27 peptide was quantified using the Caliper LabChip 3000. The kinase reactions were carried out in 20 mM HEPES pH 7.5, 10 mM MgCl₂, 0.0005% Tween-20, 0.01% BSA, 1 mM DTT, and 2% DMSO. The inhibitors were varied between 1000-0.05 nM, while the Hsp27 peptide substrate, MgATP, and nonactive MK-2 (54-400) were held constant at 0.5 □M, 5 □M, and 1 nM, respectively. Inhibitors were preincubated with p38α for 40 minutes prior to initiating the reaction with peptide, MgATP, and MK-2. The kinase reactions were quenched after 60 minutes by the addition of stop buffer (180 mM HEPES, 30 mM EDTA, and 0.2% Coating Reagent-3).

The above protocol assay was used to determine the IC₅₀ values for some of the compounds in the above Examples. The results are shown in Table 1.

TABLE 1

| | | p38a/MK2 |
|---|---|---|
| | | Cascade 40 min |
| Example | | Pre-Incuabtion (% |
| Number | Compound Name | Inhibition): IC50 |
| 34 | 1-(3-tert-butyl-1-{3-[2-(tetrahydro-2H-pyran-2- | 0.266 uM |
| | yloxy)ethoxy]phenyl}-1H-pyrazol-5-yl)-3-[5- | |
| | fluoro-2-({[1-(3-methoxybenzyl)-6-methyl-2-oxo- | |
| | 1,2-dihydropyridin-4-yl]oxy}methyl)benzyl]urea | |
| 35 | 1-[3-tert-butyl-1-(3-methoxyphenyl)-1H-pyrazol-5- | 0.0422 uM |
| | yl]-3-[5-fluoro-2-({[1-(3-methoxybenzyl)-6- | |
| | methyl-2-oxo-1,2-dihydropyridin-4- | |
| | yl]oxy}methyl)benzyl]urea | |
| 36 | 1-(3-tert-butyl-1-{3-[2-(tetrahydro-2H-pyran-2- | 0.142 uM (0.0619-0.328 |
| | yloxy)ethoxy]phenyl}-1H-pyrazol-5-yl)-3-[2-({[1- | n = 2) |
| | (3-methoxybenzyl)-6-methyl-2-oxo-1,2- | |
| | dihydropyridin-4-yl]oxy}methyl)benzyl]urea | |
| 37 | 1-{3-tert-butyl-1-[3-(2-hydroxyethoxy)phenyl]-1H- | 0.0293 uM |
| | pyrazol-5-yl}-3-[5-fluoro-2-({[1-(3- | (0.0115-0.0743 |
| | methoxybenzyl)-6-methyl-2-oxo-1,2- | n = 2) |
| | dihydropyridin-4-yl]oxy}methyl)benzyl]urea | |
| 38 | 1-{3-tert-butyl-1-[3-(2-hydroxyethoxy)phenyl]-1H- | 0.0311 uM |
| | pyrazol-5-yl}-3-[2-({[1-(3-methoxybenzyl)-6- | |
| | methyl-2-oxo-1,2-dihydropyridin-4- | |
| | yl]oxy}methyl)benzyl]urea | |
| 39 | 1-{3-tert-butyl-1-[3-(2-hydroxyethoxy)phenyl]-1H- | 0.00730 uM |
| | pyrazol-5-yl}-3-[2-({[3-chloro-1-(3- | |
| | methoxybenzyl)-6-methyl-2-oxo-1,2- | |
| | dihydropyridin-4-yl]oxy}methyl)-5- | |
| | fluorobenzyl]urea | |
| 41 | 1-(3-tert-butyl-1-{3-[2-(tetrahydro-2H-pyran-2- | 0.0216 uM |
| | yloxy)ethoxy]phenyl}-1H-pyrazol-5-yl)-3-[2-({[3- | |
| | chloro-1-(3-methoxybenzyl)-6-methyl-2-oxo-1,2- | |
| | dihydropyridin-4-yl]oxy}methyl)-5- | |
| | fluorobenzyl]urea | |
| 42 | 1-[3-tert-butyl-1-(4-{[tert- | 0.116 uM |
| | butyl(dimethyl)silyl]oxy}-3-chlorophenyl)-1H- | |
| | pyrazol-5-yl]-3-[2-({[3-chloro-1-(3- | |
| | methoxybenzyl)-6-methyl-2-oxo-1,2- | |
| | dihydropyridin-4-yl]oxy}methyl)-5- | |
| | fluorobenzyl]urea | |
| 43 | 1-[3-tert-butyl-1-(3-{[tert- | 0.0431 uM |
| | butyl(dimethyl)silyl]oxy}-4-chlorophenyl)-1H- | |
| | pyrazol-5-yl]-3-[2-({[3-chloro-1-(3- | |
| | methoxybenzyl)-6-methyl-2-oxo-1,2- | |
| | dihydropyridin-4-yl]oxy}methyl)-5- | |
| | fluorobenzyl]urea | |
| 44 | 1-[3-tert-butyl-1-(3-methoxyphenyl)-1H-pyrazol-5- | 0.00580 uM |
| | yl]-3-[2-({[3-chloro-1-(3-methoxybenzyl)-6- | |
| | methyl-2-oxo-1,2-dihydropyridin-4- | |
| | yl]oxy}methyl)benzyl]urea | |
| 45 | 1-[3-tert-butyl-1-(4-chloro-3-hydroxyphenyl)-1H- | 0.00620 uM |
| | pyrazol-5-yl]-3-[2-({[3-chloro-1-(3- | |
| | methoxybenzyl)-6-methyl-2-oxo-1,2- | |
| | dihydropyridin-4-yl]oxy}methyl)-5- | |
| | fluorobenzyl]-urea | |
| 46 | 1-[3-tert-butyl-1-(3-chloro-4-hydroxyphenyl)-1H- | 0.00400 uM |
| | pyrazol-5-yl]-3-[2-({[3-chloro-1-(3- | |
| | methoxybenzyl)-6-methyl-2-oxo-1,2- | |
| | dihydropyridin-4-yl]oxy}methyl)-5- | |
| | fluorobenzyl]urea | |
| 47 | 1-[3-tert-butyl-1-(4-chloro-3-hydroxyphenyl)-1H- | 0.0122 uM |
| | pyrazol-5-yl]-3-[2-({[1-(3-methoxybenzyl)-3,6- | |
| | dimethyl-2-oxo-1,2-dihydropyridin-4-yl]oxy}- | |
| | methyl)benzyl]urea | |
| 48 | 1-{3-tert-butyl-1-[3-(2-hydroxyethoxy)phenyl]-1H- | 0.00640 uM |
| | pyrazol-5-yl}-3-[2-({[1-(3-methoxybenzyl)-3,6- | |
| | dimethyl-2-oxo-1,2-dihydropyridin-4- | |
| | yl]oxy}methyl)benzyl]urea | |
| 50 | 1-[3-tert-butyl-1-(3-hydroxyphenyl)-1H-pyrazol-5- | 0.00650 uM |
| | yl]-3-[2-({[3-chloro-1-(3-methoxybenzyl)-6- | |
| | methyl-2-oxo-1,2-dihydropyridin-4- | |
| | yl]oxy}methyl)-5-fluoro-benzyl]urea | |
| 51 | 1-[3-tert-butyl-1-(4-hydroxyphenyl)-1H-pyrazol-5- | 0.00580 uM |
| | yl]-3-[2-({[3-chloro-1-(3-methoxybenzyl)-6- | |
| | methyl-2-oxo-1,2-dihydropyridin-4- | |
| | yl]oxy}methyl)benzyl]-urea | |
| 52 | 1-[3-tert-butyl-1-(3-hydroxyphenyl)-1H-pyrazol-5- | 0.00620 uM |
| | yl]-3-[2-({[3-chloro-1-(3-methoxybenzyl)-6- | |
| | methyl-2-oxo-1,2-dihydropyridin-4- | |
| | yl]oxy}methyl)-benzyl]urea | |
| 54 | 1-[3-tert-butyl-1-(4-hydroxyphenyl)-1H-pyrazol-5- | 0.117 uM |
| | yl]-3-[2-({[1-(4-methoxybenzyl)-6-methyl-2-oxo- | |
| | 1,2-dihydropyridin-4-yl]oxy}methyl)benzyl]urea | |
| 55 | 1-[3-tert-butyl-1-(3-hydroxyphenyl)-1H-pyrazol-5- | 0.0243 uM |
| | yl]-3-[2-({[1-(4-methoxybenzyl)-6-methyl-2-oxo- | |
| | 1,2-dihydropyridin-4-yl]oxy}methyl)benzyl]urea | |
| 56 | 1-[3-tert-butyl-1-(3-chloro-4-hydroxyphenyl)-1H- | 0.0392 uM |
| | pyrazol-5-yl]-3-[2-({[1-(4-methoxybenzyl)-6- | |
| | methyl-2-oxo-1,2-dihydropyridin-4- | |
| | yl]oxy}methyl)benzyl]urea | |
| 57 | 1-{3-tert-butyl-1-[3-(2-hydroxyethoxy)phenyl]-1H- | 0.00930 uM |
| | pyrazol-5-yl}-3-[2-({[3-chloro-1-(4- | |
| | methoxybenzyl)-6-methyl-2-oxo-1,2- | |
| | dihydropyridin-4-yl]oxy}methyl)-benzyl]urea | |
| 58 | 1-[3-tert-butyl-1-(3-chloro-4-hydroxyphenyl)-1H- | 0.00460 uM |
| | pyrazol-5-yl]-3-[2-({[3-chloro-1-(4- | |
| | methoxybenzyl)-6-methyl-2-oxo-1,2- | |
| | dihydropyridin-4-yl] oxy}methyl)benzyl]urea | |
| 59 | methyl 3-{4-[(2-{[({[3-tert-butyl-1-(4- | 0.00955 uM |
| | methylphenyl)-1H-pyrazol-5- | (0.0000397-2.29 |
| | yl]amino}carbonyl)amino]methyl}-4- | n = 2) |
| | fluorobenzyl)oxy]-2-(methylthio)-6-oxopyrimidin- | |
| | 1(6H)-yl}-4-methylbenzoate | |
| 60 | 1-{3-tert-butyl-1-[3-(2-hydroxyethoxy)phenyl]-1H- | 0.00370 uM |
| | pyrazol-5-yl}-3-[2-({[3-chloro-1-(4- | |
| | methoxybenzyl)-6-methyl-2-oxo-1,2- | |
| | dihydropyridin-4-yl]oxy}methyl)-5- | |
| | fluorobenzyl]urea | |
| 62 | 1-[3-tert-butyl-1-(3-chloro-4-hydroxyphenyl)-1H- | 0.00450 uM |
| | pyrazol-5-yl]-3-[2-({[3-chloro-1-(4- | |
| | methoxybenzyl)-6-methyl-2-oxo-1,2- | |
| | dihydropyridin-4-yl] | |
| | oxy}methyl)-5-fluorobenzyl]urea | |
| 64 | 1-[3-tert-butyl-1-(4-hydroxyphenyl)-1H-pyrazol-5- | 0.00750 uM |
| | yl]-3-[2-({[3-chloro-1-(3-chloro-4- | |
| | methoxybenzyl)-6-methyl-2-oxo-1,2- | |
| | dihydropyridin-4 yl]oxy}methyl)-benzyl]urea | |
| 65 | 1-[3-tert-butyl-1-(3-hydroxyphenyl)-1H-pyrazol-5- | 0.00690 uM |
| | yl]-3-[2-({[3-chloro-1-(3-chloro-4- | |
| | methoxybenzyl)-6-methyl-2-oxo-1,2- | |
| | dihydropyridin-4-yl]oxy}methyl)-benzyl]urea | |
| 66 | 1-[3-tert-butyl-1-(3-chloro-4-hydroxyphenyl)-1H- | 0.00970 uM |
| | pyrazol-5-yl]-3-[2-({[3-chloro-1-(3-chloro-4- | |
| | methoxybenzyl)-6-methyl-2-oxo-1,2- | |
| | dihydropyridin-4-yl]oxy}methyl)benzyl]urea | |
| 67 | 1-[3-tert-butyl-1-(4-chloro-3-hydroxyphenyl)-1H- | 0.00790 uM |
| | pyrazol-5-yl]-3-[2-({[3-chloro-1-(3-chloro-4- | |
| | methoxybenzyl)-6-methyl-2-oxo-1,2- | |
| | dihydropyridin-4-yl]oxy}methyl)benzyl]urea | |
| 81 | 1-[3-tert-butyl-1-(3-hydroxyphenyl)-1H-pyrazol-5- | 0.0201 uM |
| | yl]-3-[2-({[3-chloro-1-(4-chloro-3- | |
| | methoxybenzyl)-6-methyl-2-oxo-1,2- | |
| | dihydropyridin-4-yl]oxy}methyl)-benzyl]urea | |
| 97 | 1-[2-({[3-bromo-1-(4-methoxybenzyl)-6-methyl-2- | 0.00390 uM |
| | oxo-1,2-dihydropyridin-4-yl]oxy}methyl)benzyl]- | (0.000473-0.0321 |
| | 3-[3-tert-butyl-1-(3-fluorophenyl)-1H-pyrazol-5- | n = 2) |
| | yl]urea | |
| 98 | 1-[2-({[3-bromo-1-(4-methoxybenzyl)-6-methyl-2- | 0.00423 uM |
| | oxo-1,2-dihydropyridin-4-yl]oxy}methyl)benzyl]- | (0.00110-0.0163 |
| | 3-[3-tert-butyl-1-(3-chlorophenyl)-1H-pyrazol-5- | n = 2) |
| | yl]urea | |
| 99 | 1-[2-({[3-bromo-1-(4-methoxybenzyl)-6-methyl-2- | 0.00349 uM |
| | oxo-1,2-dihydropyridin-4-yl]oxy}methyl)benzyl]- | (0.00117-0.0104 |
| | 3-[3-tert-butyl-1-(3-methoxyphenyl)-1H-pyrazol-5- | n = 2) |
| | yl]urea | |
| 100 | 1-[2-({[3-bromo-1-(4-hydroxybenzyl)-6-methyl-2- | 0.00459 uM |
| | oxo-1,2-dihydropyridin-4-yl]oxy}methyl)benzyl]- | (0.000995-0.0212 |
| | 3-[3-tert-butyl-1-(4-methylphenyl)-1H-pyrazol-5- | n = 3) |
| | yl]urea | |
| 101 | 1-[2-({[3-bromo-1-(4-methoxybenzyl)-6-methyl-2- | 0.00819 uM |
| | oxo-1,2-dihydropyridin-4-yl]oxy}methyl)benzyl]- | (0.00441-0.0152 |
| | 3-[3-tert-butyl-1-(4-methylphenyl)-1H-pyrazol-5- | n = 2) |
| | yl]urea | |
| 102 | 1-[2-({[3-bromo-1-(4-methoxybenzyl)-6-methyl-2- | 0.182 uM |
| | oxo-1,2-dihydropyridin-4-yl]oxy}methyl)benzyl]- | |
| | 3-(3-cyclopropyl-1-phenyl-1H-pyrazol-5-yl)urea | |
| 103 | 1-(2-{[(1-benzyl-3-bromo-6-methyl-2-oxo-1,2- | 0.00262 uM |
| | dihydropyridin-4-yl)oxy]methyl}benzyl)-3-[3-tert- | (0.0000227-0.301 |
| | butyl-1-(3-fluorophenyl)-1H-pyrazol-5-yl]urea | n = 2) |
| 104 | 1-(2-{[(1-benzyl-3-bromo-6-methyl-2-oxo-1,2- | 0.00452 uM |
| | dihydropyridin-4-yl)oxy]methyl}benzyl)-3-(3-tert- | (0.000966-0.0211 |
| | butyl-1-phenyl-1H-pyrazol-5-yl)urea | n = 2) |
| 105 | 1-(2-{[(1-benzyl-3-bromo-6-methyl-2-oxo-1,2- | 0.00819 uM |
| | dihydropyridin-4-yl)oxy]methyl}benzyl)-3-[3-tert- | (0.000644-0.104 |
| | butyl-1-(2,4-difluorophenyl)-1H-pyrazol-5-yl]urea | n = 2) |
| 106 | 1-(2-{[(1-benzyl-3-bromo-6-methyl-2-oxo-1,2- | 0.00459 uM |
| | dihydropyridin-4-yl)oxy]methyl}benzyl)-3-[3-tert- | (0.000101-0.208 |
| | butyl-1-(4-methylphenyl)-1H-pyrazol-5-yl]urea | n = 2) |
| 107 | 1-(2-{[(1-benzyl-3-bromo-6-methyl-2-oxo-1,2- | 0.176 uM |
| | dihydropyridin-4-yl)oxy]methyl}benzyl)-3-(3- | |
| | cyclopropyl-1-phenyl-1H-pyrazol-5-yl)urea | |
| 108 | 1-(2-{[(1-benzyl-3-bromo-6-methyl-2-oxo-1,2- | 0.00371 uM |
| | dihydropyridin-4-yl)oxy]methyl}benzyl)-3-[3-tert- | (0.000568-0.0242 |
| | butyl-1-(3-chlorophenyl)-1H-pyrazol-5-yl]urea | n = 2) |
| 109 | 1-(2-{[(1-benzyl-3-bromo-6-methyl-2-oxo-1,2- | 0.00638 uM |
| | dihydropyridin-4-yl)oxy]methyl}benzyl)-3-[3-tert- | (0.000969-0.0420 |
| | butyl-1-(3-methoxyphenyl)-1H-pyrazol-5-yl]urea | n = 2) |
| 110 | 1-(2-{[(1-benzyl-5-ethyl-6-oxo-1,6- | 0.0144 uM |
| | dihydropyrimidin-4-yl)oxy]methyl}benzyl)-3-[3- | (0.000525-0.395 |
| | tert-butyl-1-(4-methylphenyl)-1H-pyrazol-5-yl]urea | n = 2) |
| 111 | 1-(2-{[(1-benzyl-5-ethyl-6-oxo-1,6- | 0.0110 uM |
| | dihydropyrimidin-4-yl)oxy]methyl}benzyl)-3-[3- | (0.00166-0.0732 |
| | tert-butyl-1-(3-fluorophenyl)-1H-pyrazol-5-yl]urea | n = 2) |
| 113 | 1-(2-{[(1-benzyl-5-ethyl-6-oxo-1,6- | 0.0100 uM |
| | dihydropyrimidin-4-yl)oxy]methyl}benzyl)-3-(3- | (0.00645-0.0156 |
| | tert-butyl-1-phenyl-1H-pyrazol-5-yl)urea | n = 2) |
| 114 | 1-[3-tert-butyl-1-(4-methylphenyl)-1H-pyrazol-5- | 0.0163 uM |
| | yl]-3-[2-({[5-ethyl-1-(4-methoxybenzyl)-6-oxo-1,6- | (0.000509-0.521 |
| | dihydropyrimidin-4-yl]oxy}methyl)benzyl]urea | n = 2) |
| 115 | 1-[3-tert-butyl-1-(3-methoxyphenyl)-1H-pyrazol-5- | 0.0258 uM |
| | yl]-3-[2-({[5-ethyl-1-(4-methoxybenzyl)-6-oxo-1,6- | (0.00236-0.283 |
| | dihydropyrimidin-4-yl]oxy}methyl)benzyl]urea | n = 2) |
| 116 | 1-[3-tert-butyl-1-(3-fluorophenyl)-1H-pyrazol-5- | 0.0101 uM |
| | yl]-3-[2-({[5-ethyl-1-(4-methoxybenzyl)-6-oxo-1,6- | (0.00135-0.0751 |
| | dihydropyrimidin-4-yl]oxy}methyl)benzyl]urea | n = 2) |
| 117 | 1-[2-({[3-bromo-1-(2,4-dimethoxybenzyl)-6- | 0.0157 uM |
| | methyl-2-oxo-1,2-dihydropyridin-4- | (0.00215-0.115 |
| | yl]oxy}methyl)benzyl]-3-[3-tert-butyl-1-(3- | n = 3) |
| | fluorophenyl)-1H-pyrazol-5-yl]urea | |
| 119 | 1-[2-({[3-bromo-1-(2,4-dimethoxybenzyl)-6- | 0.0119 uM |
| | methyl-2-oxo-1,2-dihydropyridin-4- | (0.00916-0.0156 |
| | yl]oxy}methyl)benzyl]-3-[3-tert-butyl-1-(3- | n = 2) |
| | methoxyphenyl)-1H-pyrazol-5-yl]urea | |
| 120 | 1-[2-({[3-bromo-1-(2,4-dimethoxybenzyl)-6- | 0.0155 uM |
| | methyl-2-oxo-1,2-dihydropyridin-4- | (0.0103-0.0233 |
| | yl]oxy}methyl)benzyl]-3-(3-tert-butyl-1-phenyl- | n = 2) |
| | 1H-pyrazol-5-yl)urea | |
| 121 | 1-[2-({[3-bromo-1-(2,4-dimethoxybenzyl)-6- | 0.0110 uM |
| | methyl-2-oxo-1,2-dihydropyridin-4- | (0.00873-0.0139 |
| | yl]oxy}methyl)benzyl]-3-[3-tert-butyl-1-(2,4- | n = 2) |
| | difluorophenyl)-1H-pyrazol-5-yl]urea | |
| 122 | 1-[2-({[3-bromo-1-(2,4-dimethoxybenzyl)-6- | 0.0154 uM |
| | methyl-2-oxo-1,2-dihydropyridin-4- | (0.00394-0.0599 |
| | yl]oxy}methyl)benzyl]-3-[3-tert-butyl-1-(4- | n = 2) |
| | methylphenyl)-1H-pyrazol-5-yl]urea | |
| 123 | 1-[3-tert-butyl-1-(3-methoxyphenyl)-1H-pyrazol-5- | 0.0111 uM |
| | yl]-3-[2-({[1-(2,4-dimethoxybenzyl)-3,6-dimethyl- | |
| | 2-oxo-1,2-dihydropyridin-4- | |
| | yl]oxy}methyl)benzyl]urea | |
| 128 | 1-[3-tert-butyl-1-(3-methoxyphenyl)-1H-pyrazol-5- | 0.0169 uM |
| | yl]-3-[2-({[1-(4-methoxybenzyl)-3,6-dimethyl-2- | (0.00172-0.165 |
| | oxo-1,2-dihydropyridin-4- | n = 2) |
| | yl]oxy}methyl)benzyl]urea | |
| 128-B | 1-(2-{[(1-benzyl-3,6-dimethyl-2-oxo-1,2- | 0.0125 uM |
| | dihydropyridin-4-yl)oxy]methyl}benzyl)-3-[3-tert- | (0.00193-0.0813 |
| | butyl-1-(3-methoxyphenyl)-1H-pyrazol-5-yl]urea | n = 2) |
| 128-C | 1-(2-{[(1-benzyl-3,6-dimethyl-2-oxo-1,2- | 0.0121 uM |
| | dihydropyridin-4-yl)oxy]methyl}benzyl)-3-[3-tert- | (0.000628-0.234 |
| | butyl-1-(4-methylphenyl)-1H-pyrazol-5-yl]urea | n = 2) |
| 156 | 3-[4-(2-{3-[5-tert-butyl-2-(3-chloro-4-hydroxy- | 0.00350 uM |
| | phenyl)-2H-pyrazol-3-yl]-ureidomethyl}- | |
| | benzyloxy)-3-chloro-6-methyl-2-oxo-2H-pyridin-1- | |
| | yl]-4-methyl-N-methylcarbamoylmethyl- | |
| | benzamide | |
| 159 | (R)-3-[4-(2-{3-[5-tert-butyl-2-(3-chloro-4-hydroxy- | 0.00250 uM |
| | phenyl)-2H-pyrazol-3-yl]-ureidomethyl}- | |
| | benzyloxy)-3-chloro-6-methyl-2-oxo-2H-pyridin-1- | |
| | yl]-N-(1-carbamoyl-ethyl)-4-methyl-benzamide | |
| 167 | 3-[4-(2-{3-[5-tert-Butyl-2-(4-chloro-3-hydroxy- | 0.00170 uM |
| | phenyl)-2H-pyrazol-3-yl]-ureidomethyl}- | |
| | benzyloxy)-3-chloro-6-methyl-2-oxo-2H-pyridin-1- | |
| | yl]-4-methyl-N-methylcarbamoylmethyl- | |
| | benzamide | |
| 178 | 3-{4-[2-(3-{5-tert-butyl-2-[3-(2-hydroxy-ethoxy)- | 0.00140 uM |
| | phenyl]-2H-pyrazol-3-yl}-ureidomethyl)-4-fluoro- | |
| | benzyloxy]-3-chloro-6-methyl-2-oxo-2H-pyridin-1- | |
| | yl}-4-methyl-benzoic acid 2-dimethylamino- | |
| | ethylester | |
| 180 | 3-{4-[2-(3-{5-tert-butyl-2-[3-(2-hydroxy-ethoxy)- | 0.00170 uM |
| | phenyl]-2H-pyrazol-3-yl}-ureidomethyl)-4-fluoro- | |
| | benzyloxy]-3-chloro-6-methyl-2-oxo-2H-pyridin-1- | |
| | yl}-4-methyl-benzoic acid 2-hydroxy-ethyl ester | |
| 181 | methyl 3-{4-[(2-{[({[3-tert-butyl-1-(4- | 0.00331 uM |
| | methylphenyl)-1H-pyrazol-5- | (0.000924-0.0119 |
| | yl]amino}carbonyl)amino]methyl}-4- | n = 3) |
| | fluorobenzyl)oxy]-3-chloro-6-methyl-2- | |
| | oxopyridin-1(2H)-yl}-4-methylbenzoate | |
| 182 | methyl 3-{4-[(2-{[({[3-tert-butyl-1-(3- | 0.00399 uM |
| | chlorophenyl)-1H-pyrazol-5- | (0.00154-0.0104 |
| | yl]amino}carbonyl)amino]methyl}-4- | n = 2) |
| | fluorobenzyl)oxy]-3-chloro-6-methyl-2-oxopyridin- | |
| | 1(2H)-yl}-4-methylbenzoate | |
| 183 | methyl 3-{4-[(2-{[({[3-tert-butyl-1-(3- | 0.00226 uM |
| | methoxyphenyl)-1H-pyrazol-5- | (0.0000613-0.0832 |
| | yl]amino}carbonyl)amino]methyl}-4- | n = 2) |
| | fluorobenzyl)oxy]-3-chloro-6-methyl-2-oxopyridin- | |
| | 1(2H)-yl}-4-methylbenzoate | |
| 185 | methyl 3-{3-bromo-4-[(2-{[({[3-tert-butyl-1-(4- | 0.00361 uM |
| | methylphenyl)-1H-pyrazol-5- | (0.00187-0.00696 |
| | yl]amino}carbonyl)amino]methyl}-4- | n = 3) |
| | fluorobenzyl)oxy]-6-methyl-2-oxopyridin-1(2H)- | |
| | yl}-4-methylbenzoate | |
| 186 | 3-{4-[(2-{[({[3-tert-butyl-1-(4-methylphenyl)-1H- | 0.00462 uM |
| | pyrazol-5-yl]amino}carbonyl)amino]methyl}-4- | (0.00296-0.00721 |
| | fluoro-benzyl)oxy]-3-chloro-6-methyl-2- | n = 4) |
| | oxopyridin-1(2H)-yl}-4-methylbenzoic acid | |
| 189 | 3-{4-[(2-{[({[3-tert-butyl-1-(3-methoxyphenyl)- | 0.00255 uM |
| | 1H-pyrazol-5-yl]amino}carbonyl)amino]methyl}- | (0.00120-0.00538 |
| | 4-fluoro-benzyl)oxy]-3-chloro-6-methyl-2- | n = 2) |
| | oxopyridin-1(2H)-yl}-4-methylbenzoic acid | |
| 190 | 3-{4-[(2-{[({[3-tert-butyl-1-(4-methylphenyl)-1H- | 0.00237 uM |
| | pyrazol-5-yl]amino}carbonyl)amino]methyl}-4- | (0.00122-0.00463 |
| | fluoro-benzyl)oxy]-3-chloro-6-methyl-2- | n = 3) |
| | oxopyridin-1(2H)-yl}-N-(2-hydroxyethyl)-4- | |
| | methylbenzamide | |
| 193 | 3-{3-bromo-4-[(2-{[({[3-tert-butyl-1-(4-methyl- | 0.00221 uM |
| | phenyl)-1H-pyrazol-5-yl]amino}carbonyl)- | (0.000727-0.00670 |
| | amino]methyl}-4-fluorobenzyl)oxy]-6-methyl-2- | n = 4) |
| | oxopyridin-1(2H)-yl}-N-(2-hydroxyethyl)-4- | |
| | methyl-benzamide | |
| 194 | 3-{4-[(2-{[({[3-tert-butyl-1-(3-chlorophenyl)-1H- | 0.00245 uM |
| | pyrazol-5-yl]amino}carbonyl)amino]methyl}-4- | (0.00112-0.00533 |
| | fluorobenzyl)oxy]-3-chloro-6-methyl-2-oxopyridin- | n = 2) |
| | 1(2H)-yl}-N,4-dimethylbenzamide | |
| 195 | 3-{4-[(2-{[({[3-tert-butyl-1-(3-chlorophenyl)-1H- | 0.00393 uM |
| | pyrazol-5-yl]amino}carbonyl)amino]methyl}-4- | (0.00127-0.0122 |
| | fluorobenzyl)oxy]-3-chloro-6-methyl-2-oxopyridin- | n = 2) |
| | 1(2H)-yl}-N-(2-hydroxyethyl)-4-methylbenzamide | |

IC50 values were determined for the compounds listed in Table 2, using the same p38α/MK2 cascade assay used to determine the IC50 values for the compounds in Table 1. The compounds of Table 2 were prepared using methods similar to those listed in the Examples for compounds of Table 1.

TABLE 2

| | | p38a/MK2 |
|---|---|---|
| | | Cascade 40 min |
| | | Pre-Incuabtion |
| Example | | (% |
| Number | Compound Name | Inhibition): IC50 |
| 201 | 1-[3-tert-butyl-1-(3-methoxyphenyl)-1H-pyrazol-5-yl]-3-[2-({[3- | 0.00450 uM |
| | chloro-1-(3-methoxybenzyl)-6-methyl-2-oxo-1,2-dihydropyridin- | |
| | 4-yl]oxy}methyl)-5-fluoro-benzyl]urea | |
| 202 | 1-[3-tert-butyl-1-(4-chloro-3-hydroxyphenyl)-1H-pyrazol-5-yl]-3- | 0.00310 uM |
| | [2-({[3-chloro-1-(4-methoxy-benzyl)-6-methyl-2-oxo-1,2- | |
| | dihydropyridin-4-yl]oxy}methyl)-5-fluorobenzyl]urea | |
| 203 | methyl 3-{4-[(2-{[({[3-tert-butyl-1-(3-hydroxy-phenyl)-1H- | 0.00280 uM |
| | pyrazol-5-yl]amino}-carbonyl)amino]-methyl}-4- | |
| | fluorobenzyl)oxy]-3-chloro-6-methyl-2-oxopyridin-1(2H)-yl}-4- | |
| | methylbenzoate | |
| 204 | methyl 3-{4-[(2-{[({[3-tert-butyl-1-(4-methylphenyl)-1H-pyrazol- | 0.0263 uM |
| | 5-yl]amino}carbonyl)-amino]-methyl}-benzyl)oxy]-2- | |
| | (methylthio)-6-oxopyrimidin-1(6H)-yl}-4-methylbenzoate | |
| 205 | methyl 3-{4-[(2-{[({[3-tert-butyl-1-(4-methylphenyl)-1H-pyrazol- | 0.00250 uM |
| | 5-yl]amino}carbonyl)-amino]methyl}benzyl)oxy]-3-chloro-6- | (0.0000752-0.834 |
| | methyl-2-oxopyridin-1(2H)-yl}-4-methylbenzoate | n = 2) |
| 206 | 3-{4-[(2-{[({[3-tert-butyl-1-(4-methylphenyl)-1H-pyrazol-5- | 0.00371 uM |
| | yl]amino}carbonyl)amino]methyl}-4-fluorobenzyl)oxy]-3-chloro- | (0.00265-0.00519 |
| | 6-methyl-2-oxopyridin-1(2H)-yl}-N,4-dimethylbenzamide | n = 3) |
| 207 | 3-{3-bromo-4-[(2-{[({[3-tert-butyl-1-(4-methylphenyl)-1H- | 0.00454 uM |
| | pyrazol-5-yl]amino}carbonyl)-amino]methyl}-4- | (0.00226-0.00914 |
| | fluorobenzyl)oxy]-6-methyl-2-oxopyridin-1(2H)-yl}-4- | n = 2) |
| | methylbenzoic acid | |
| 208 | 3-{4-[(2-{[({[3-tert-butyl-1-(3-methoxyphenyl)-1H-pyrazol-5- | 0.00179 uM |
| | yl]amino}carbonyl)amino]methyl}-4-fluorobenzyl)oxy]-3-chloro- | (0.000434-0.00738 |
| | 6-methyl-2-oxopyridin-1(2H)-yl}-N,4-dimethyl-benzamide | n = 2) |
| 209 | methyl 3-[4-{[2-({[({3-tert-butyl-1-[3-(2-hydroxyethoxy)- | 0.00380 uM |
| | phenyl]-1H-pyrazol-5-yl}amino)carbonyl]-amino}methyl)- | |
| | benzyl]oxy}-3-chloro-6-methyl-2-oxopyridin-1(2H)-yl]-4- | |
| | methylbenzoate | |
| 210 | 3-[4-{[2-({[({3-tert-butyl-1-[3-(2-hydroxyethoxy)phenyl]-1H- | 0.00290 uM |
| | pyrazol-5-yl}amino)carbonyl]amino}methyl)-benzyl]oxy}-3- | |
| | chloro-6-methyl-2-oxopyridin-1(2H)-yl]-N-(2-hydroxyethyl)-4- | |
| | methylbenzamide | |
| 211 | 3-[4-{[2-({[({3-tert-butyl-1-[3-(2-hydroxyethoxy)phenyl]-1H- | 0.00260 uM |
| | pyrazol-5-yl}amino)carbonyl]amino}methyl)benzyl]oxy}-3- | |
| | chloro-6-methyl-2-oxopyridin-1(2H)-yl]-N,4-dimethylbenzamide | |
| 212 | 3-[4-{[2-({[({3-tert-butyl-1-[3-(2-hydroxyethoxy)phenyl]-1H- | 0.00180 uM |
| | pyrazol-5-yl}amino)carbonyl]amino}methyl)benzyl]oxy}-3- | |
| | chloro-6-methyl-2-oxopyridin-1(2H)-yl]-N-(2,3- | |
| | dihydroxypropyl)-4-methylbenzamide | |
| 213 | methyl 3-{4-[(2-{[({[3-tert-butyl-1-(4-hydroxyphenyl)-1H- | 0.00400 uM |
| | pyrazol-5-yl]amino}carbonyl)amino]methyl}benzyl)oxy]-3- | |
| | chloro-6-methyl-2-oxopyridin-1(2H)-yl}-4-methylbenzoate | |
| 214 | 3-{4-[(2-{[({[3-tert-butyl-1-(4-hydroxyphenyl)-1H-pyrazol-5- | 0.00210 uM |
| | yl]amino}carbonyl)amino]methyl}benzyl)oxy]-3-chloro-6- | |
| | methyl-2-oxopyridin-1(2H)-yl}-N,4-dimethylbenzamide | |
| 215 | 1-[3-tert-butyl-1-(3-chloro-4-hydroxyphenyl)-1H-pyrazol-5-yl]-3- | 0.00800 uM |
| | [2-({[3-chloro-1-(3-methoxy-benzyl)-6-methyl-2-oxo-1,2- | |
| | dihydropyridin-4-yl]oxy}methyl)-benzyl]urea | |
| 216 | 3-{4-[(2-{[({[3-tert-butyl-1-(4-hydroxyphenyl)-1H-pyrazol-5- | 0.00180 uM |
| | yl]amino}carbonyl)amino]methyl}benzyl)oxy]-3-chloro-6- | |
| | methyl-2-oxopyridin-1(2H)-yl}-N-(2-hydroxyethyl)-4-methyl- | |
| | benzamide | |
| 217 | 3-{4-[(2-{[({[3-tert-butyl-1-(4-hydroxyphenyl)-1H-pyrazol-5- | 0.00150 uM |
| | yl]amino}carbonyl)amino]methyl}benzyl)oxy]-3-chloro-6- | |
| | methyl-2-oxopyridin-1(2H)-yl}-N-(2,3-dihydroxypropyl)-4- | |
| | methylbenzamide | |
| 218 | 3-{4-[(2-{[({[3-tert-butyl-1-(4-hydroxyphenyl)-1H-pyrazol-5- | 0.00181 uM |
| | yl]amino}carbonyl)amino]methyl}benzyl)oxy]-3-chloro-6- | (0.00129-0.00254 |
| | methyl-2-oxopyridin-1(2H)-yl}-N-(2-methoxyethyl)-4-methyl- | n = 5) |
| | benzamide | |
| 219 | N-(2-amino-2-oxoethyl)-3-{4-[(2-{[({[3-tert-butyl-1-(4- | 0.000900 uM |
| | hydroxyphenyl)-1H-pyrazol-5-yl]amino}carbonyl)amino]- | |
| | methyl}benzyl)oxy]-3-chloro-6-methyl-2-oxopyridin-1(2H)-yl}- | |
| | 4-methylbenzamide | |
| 220 | N-[(1S)-2-amino-1-methyl-2-oxoethyl]-3-{4-[(2-{[({[3-tert-butyl- | 0.00140 uM |
| | 1-(4-hydroxyphenyl)-1H-pyrazol-5-yl]amino}carbonyl)- | |
| | amino]methyl}benzyl)oxy]-3-chloro-6-methyl-2-oxopyridin- | |
| | 1(2H)-yl}-4-methylbenzamide | |
| 221 | methyl 3-{4-[(2-{[({[3-tert-butyl-1-(4-chloro-3-hydroxy-phenyl)- | 0.0105 uM |
| | 1H-pyrazol-5-yl]amino}carbonyl)amino]-methyl}-benzyl)oxy]-3- | |
| | chloro-6-methyl-2-oxopyridin-1(2H)-yl}-4-methylbenzoate | |
| 222 | methyl 3-{4-[(2-{[({[3-tert-butyl-1-(3-chloro-4-hydroxy-phenyl)- | 0.00960 uM |
| | 1H-pyrazol-5-yl]amino}carbonyl)amino]-methyl}-benzyl)oxy]-3- | |
| | chloro-6-methyl-2-oxopyridin-1(2H)-yl}-4-methylbenzoate | |
| 223 | 3-{4-[(2-{[({[3-tert-butyl-1-(3-chloro-4-hydroxyphenyl)-1H- | 0.00460 uM |
| | pyrazol-5-yl]amino}carbonyl)amino]methyl}benzyl)oxy]-3- | |
| | chloro-6-methyl-2-oxopyridin-1(2H)-yl}-N,4-dimethylbenzamide | |
| 224 | 3-{4-[(2-{[({[3-tert-butyl-1-(3-chloro-4-hydroxyphenyl)-1H- | 0.00320 uM |
| | pyrazol-5-yl]amino}carbonyl)amino]methyl}benzyl)oxy]-3- | |
| | chloro-6-methyl-2-oxopyridin-1(2H)-yl}-N-(2-hydroxyethyl)-4- | |
| | methylbenzamide | |
| 225 | 3-{4-[(2-{[({[3-tert-butyl-1-(3-chloro-4-hydroxyphenyl)-1H- | 0.00303 uM |
| | pyrazol-5-yl]amino}carbonyl)amino]methyl}benzyl)oxy]-3- | (0.00187-0.00490 |
| | chloro-6-methyl-2-oxopyridin-1(2H)-yl}-N-(2-methoxyethyl)-4- | n = 5) |
| | methylbenzamide | |
| 226 | 3-{4-[(2-{[({[3-tert-butyl-1-(4-chloro-3-hydroxyphenyl)-1H- | 0.00360 uM |
| | pyrazol-5-yl]amino}carbonyl)amino]methyl}benzyl)oxy]-3- | |
| | chloro-6-methyl-2-oxopyridin-1(2H)-yl}-N,4-dimethylbenzamide | |
| 227 | 3-{4-[(2-{[({[3-tert-butyl-1-(3-chloro-4-hydroxyphenyl)-1H- | 0.00200 uM |
| | pyrazol-5-yl]amino}carbonyl)amino]methyl}benzyl)oxy]-3- | |
| | chloro-6-methyl-2-oxopyridin-1(2H)-yl}-N-(2,3-dihydroxy- | |
| | propyl)-4-methylbenzamide | |
| 228 | 3-{4-[(2-{[({[3-tert-butyl-1-(4-chloro-3-hydroxyphenyl)-1H- | 0.00210 uM |
| | pyrazol-5-yl]amino}carbonyl)amino]methyl}benzyl)oxy]-3- | |
| | chloro-6-methyl-2-oxopyridin-1(2H)-yl}-N-(2-hydroxyethyl)-4- | |
| | methylbenzamide | |
| 229 | 3-{4-[(2-{[({[3-tert-butyl-1-(3-chloro-4-hydroxyphenyl)-1H- | 0.00230 uM |
| | pyrazol-5-yl]amino}carbonyl)amino]methyl}benzyl)oxy]-3- | |
| | chloro-6-methyl-2-oxopyridin-1(2H)-yl}-N-[2-(dimethylamino)- | |
| | ethyl]-4-methylbenzamide | |
| 230 | 3-{4-[(2-{[({[3-tert-butyl-1-(4-chloro-3-hydroxyphenyl)-1H- | 0.00192 uM |
| | pyrazol-5-yl]amino}carbonyl)amino]methyl}benzyl)oxy]-3- | (0.00129-0.00286 |
| | chloro-6-methyl-2-oxopyridin-1(2H)-yl}-N-(2-methoxyethyl)-4- | n = 5) |
| | methylbenzamide | |
| 231 | 3-{4-[(2-{[({[3-tert-butyl-1-(4-chloro-3-hydroxyphenyl)-1H- | 0.00180 uM |
| | pyrazol-5-yl]amino}carbonyl)amino]methyl}benzyl)oxy]-3- | |
| | chloro-6-methyl-2-oxopyridin-1(2H)-yl}-N-[2-(dimethylamino)- | |
| | ethyl]-4-methylbenzamide | |
| 232 | methyl 3-{4-[(2-{[({[3-tert-butyl-1-(4-hydroxyphenyl)-1H- | 0.00440 uM |
| | pyrazol-5-yl]amino}carbonyl)amino]methyl}-4-fluorobenzyl)- | |
| | oxy]-3-chloro-6-methyl-2-oxopyridin-1(2H)-yl}-4- | |
| | methylbenzoate | |
| 233 | N-(2-amino-2-oxoethyl)-3-{4-[(2-{[({[3-tert-butyl-1-(3-chloro-4- | 0.00150 uM |
| | hydroxyphenyl)-1H-pyrazol-5-yl]amino}carbonyl)amino]- | |
| | methyl}benzyl)oxy]-3-chloro-6-methyl-2-oxopyridin-1(2H)-yl}- | |
| | 4-methylbenzamide | |
| 234 | 3-{4-[(2-{[({[3-tert-butyl-1-(4-chloro-3-hydroxyphenyl)-1H- | 0.00130 uM |
| | pyrazol-5-yl]amino}carbonyl)amino]methyl}benzyl)oxy]-3- | |
| | chloro-6-methyl-2-oxopyridin-1(2H)-yl}-N-(2,3-dihydroxy- | |
| | propyl)-4-methylbenzamide | |
| 235 | 3-{4-[(2-{[({[3-tert-butyl-1-(4-chloro-3-hydroxyphenyl)-1H- | 0.00420 uM |
| | pyrazol-5-yl]amino}carbonyl)amino]methyl}-4-fluorobenzyl)- | |
| | oxy]-3-chloro-6-methyl-2-oxopyridin-1(2H)-yl}-N-(2-methoxy- | |
| | ethyl)-4-methylbenzamide | |
| 236 | 3-{4-[(2-{[({[3-tert-butyl-1-(3-chloro-4-hydroxyphenyl)-1H- | 0.00750 uM |
| | pyrazol-5-yl]amino}carbonyl)amino]methyl}-4-fluorobenzyl)- | |
| | oxy]-3-chloro-6-methyl-2-oxopyridin-1(2H)-yl}-N,4-dimethyl- | |
| | benzamide | |
| 237 | 1-(3-tert-butyl-1-phenyl-1H-pyrazol-5-yl)-3-[2-({[1-(4- | 0.0112 uM |
| | methoxybenzyl)-3,6-dimethyl-2-oxo-1,2-dihydropyridin-4- | (0.00139-0.0904 |
| | yl]oxy}methyl)benzyl]urea | n = 2) |
| 238 | 3-[4-{[2-({[({3-tert-butyl-1-[3-(2-hydroxyethoxy)phenyl]-1H- | 0.00200 uM |
| | pyrazol-5-yl}amino)carbonyl]amino}methyl)benzyl]oxy}-3- | |
| | chloro-6-methyl-2-oxopyridin-1(2H)-yl]-N-(2-methoxyethyl)-4- | |
| | methylbenzamide | |
| 239 | 3-[4-{[2-({[({3-tert-butyl-1-[3-(2-hydroxyethoxy)phenyl]-1H- | 0.00290 uM |
| | pyrazol-5-yl}amino)carbonyl]amino}methyl)-4-fluorobenzyl]- | |
| | oxy}-3-chloro-6-methyl-2-oxopyridin-1(2H)-yl]-N-(2-hydroxy- | |
| | ethyl)-4-methylbenzamide | |
| 240 | 3-[4-{[2-({[({3-tert-butyl-1-[3-(2-hydroxyethoxy)phenyl]-1H- | 0.00210 uM |
| | pyrazol-5-yl}amino)carbonyl]amino}methyl)benzyl]oxy}-3- | |
| | chloro-6-methyl-2-oxopyridin-1(2H)-yl]-N-[2-(dimethylamino)- | |
| | ethyl]-4-methylbenzamide | |
| 241 | 1-[3-tert-butyl-1-(4-hydroxyphenyl)-1H-pyrazol-5-yl]-3-{2- | 0.0136 uM |
| | [({3,6-dimethyl-1-[2-(methylthio)benzyl]-2-oxo-1,2-dihydro- | |
| | pyridin-4-yl}oxy)methyl]benzyl}urea | |
| 242 | 1-[2-({[3-bromo-1-(2,4-dimethoxybenzyl)-6-methyl-2-oxo-1,2- | 0.0108 uM |
| | dihydropyridin-4-yl]oxy}methyl)benzyl]-3-[3-tert-butyl-1-(3- | (0.00331-0.0350 |
| | chlorophenyl)-1H-pyrazol-5-yl]urea | n = 2) |
| 243 | 1-(3-tert-butyl-1-phenyl-1H-pyrazol-5-yl)-3-[2-({[1-(2,4- | 0.00954 uM |
| | dimethoxybenzyl)-3,6-dimethyl-2-oxo-1,2-dihydropyridin-4- | (0.00599-0.0152 |
| | yl]oxy}methyl)benzyl]urea | n = 2) |
| 244 | 1-[3-tert-butyl-1-(2,4-difluorophenyl)-1H-pyrazol-5-yl]-3-[2-({[1- | 0.0137 uM |
| | (2,4-dimethoxybenzyl)-3,6-dimethyl-2-oxo-1,2-dihydropyridin-4- | |
| | yl]oxy}methyl)benzyl]urea | |
| 245 | 1-[3-tert-butyl-1-(4-methylphenyl)-1H-pyrazol-5-yl]-3-[2-({[1- | 0.0134 uM |
| | (2,4-dimethoxybenzyl)-3,6-dimethyl-2-oxo-1,2-dihydropyridin-4- | |
| | yl]oxy}methyl)benzyl]urea | |
| 246 | 1-[3-tert-butyl-1-(4-hydroxyphenyl)-1H-pyrazol-5-yl]-3-[2-({[3- | 0.0100 uM |
| | chloro-1-(2-methoxybenzyl)-6-methyl-2-oxo-1,2-dihydropyridin- | (0.00831-0.0121 |
| | 4-yl]oxy}methyl)benzyl]urea | n = 2) |
| 247 | 1-[3-tert-butyl-1-(3-hydroxyphenyl)-1H-pyrazol-5-yl]-3-[2-({[3- | 0.0105 uM |
| | chloro-1-(2-methoxybenzyl)-6-methyl-2-oxo-1,2-dihydropyridin- | |
| | 4-yl]oxy}methyl)benzyl]urea | |
| 248 | 1-[3-tert-butyl-1-(3-chloro-4-hydroxyphenyl)-1H-pyrazol-5-yl]-3- | 0.0151 uM |
| | [2-({[3-chloro-1-(2-methoxybenzyl)-6-methyl-2-oxo-1,2- | |
| | dihydropyridin-4-yl]oxy}methyl)benzyl]urea | |
| 249 | 1-[3-tert-butyl-1-(3-chloro-4-hydroxyphenyl)-1H-pyrazol-5-yl]-3- | 0.00960 uM |
| | [2-({[1-(3-cyano-4-methoxybenzyl)-3,6-dimethyl-2-oxo-1,2- | |
| | dihydropyridin-4-yl]oxy}methyl)benzyl]urea | |
| 250 | 1-{3-tert-butyl-1-[3-(2-hydroxyethoxy)phenyl]-1H-pyrazol-5-yl}- | 0.00840 uM |
| | 3-[2-({[1-(3-cyano-4-methoxybenzyl)-3,6-dimethyl-2-oxo-1,2- | |
| | dihydropyridin-4-yl]oxy}methyl)benzyl]urea | |
| 251 | 1-[3-tert-butyl-1-(4-chloro-3-hydroxyphenyl)-1H-pyrazol-5-yl]-3- | 0.00840 uM |
| | [2-({[1-(3-cyano-4-methoxybenzyl)-3,6-dimethyl-2-oxo-1,2- | |
| | dihydropyridin-4-yl]oxy}methyl)benzyl]urea | |
| 252 | 1-[3-tert-butyl-1-(3-hydroxyphenyl)-1H-pyrazol-5-yl]-3-[2-({[1- | 0.00600 uM |
| | (3-cyano-4-methoxybenzyl)-3,6-dimethyl-2-oxo-1,2-dihydro- | |
| | pyridin-4-yl]oxy}methyl)benzyl]urea | |
| 253 | 1-[3-tert-butyl-1-(4-hydroxyphenyl)-1H-pyrazol-5-yl]-3-[2-({[1- | 0.00830 uM |
| | (3-cyano-4-methoxybenzyl)-3,6-dimethyl-2-oxo-1,2- | |
| | dihydropyridin-4-yl]oxy}methyl)benzyl]urea | |
| 254 | 1-[3-tert-butyl-1-(3-chloro-4-hydroxyphenyl)-1H-pyrazol-5-yl]-3- | 0.0266 uM |
| | [2-({[3-chloro-1-(4-chloro-3-methoxybenzyl)-6-methyl-2-oxo- | |
| | 1,2-dihydropyridin-4-yl]oxy}methyl)benzyl]urea | |
| 255 | 1-{3-tert-butyl-1-[3-(2-hydroxyethoxy)phenyl]-1H-pyrazol-5-yl}- | 0.00720 uM |
| | 3-[2-({[3-chloro-1-(4-chloro-3-methoxybenzyl)-6-methyl-2-oxo- | |
| | 1,2-dihydropyridin-4-yl]oxy}methyl)benzyl]urea | |
| 256 | 1-[3-tert-butyl-1-(4-chloro-3-hydroxyphenyl)-1H-pyrazol-5-yl]-3- | 0.0199 uM |
| | [2-({[3-chloro-1-(4-chloro-3-methoxybenzyl)-6-methyl-2-oxo- | |
| | 1,2-dihydropyridin-4-yl]oxy}methyl)benzyl]urea | |
| 257 | 1-[3-tert-butyl-1-(4-chloro-3-hydroxyphenyl)-1H-pyrazol-5-yl]-3- | 0.00470 uM |
| | [2-({[3-chloro-1-(4-methoxybenzyl)-6-methyl-2-oxo-1,2-dihydro- | |
| | pyridin-4-yl]oxy}methyl)benzyl]urea | |
| 258 | 1-[3-tert-butyl-1-(4-hydroxyphenyl)-1H-pyrazol-5-yl]-3-[2-({[3- | 0.0101 uM |
| | chloro-1-(4-chloro-3-methoxybenzyl)-6-methyl-2-oxo-1,2- | |
| | dihydropyridin-4-yl]oxy}methyl)benzyl]urea | |
| 259 | 1-[3-tert-butyl-1-(3-hydroxyphenyl)-1H-pyrazol-5-yl]-3-[2-({[1- | 0.00920 uM |
| | (3-chloro-4-methoxybenzyl)-3,6-dimethyl-2-oxo-1,2-dihydro- | |
| | pyridin-4-yl]oxy}methyl)benzyl]urea | |
| 260 | 1-[3-tert-butyl-1-(4-chloro-3-hydroxyphenyl)-1H-pyrazol-5-yl]-3- | 0.0154 uM |
| | [2-({[1-(3-chloro-4-methoxybenzyl)-3,6-dimethyl-2-oxo-1,2- | |
| | dihydropyridin-4-yl]oxy}methyl)benzyl]urea | |
| 261 | N-[3-({4-[(2-{[({[3-tert-butyl-1-(3-chloro-4-hydroxyphenyl)-1H- | 0.00890 uM |
| | pyrazol-5-yl]amino}carbonyl)amino]methyl}benzyl)oxy]-3- | |
| | chloro-6-methyl-2-oxopyridin-1(2H)-yl}methyl)benzyl]-2- | |
| | hydroxyacetamide | |
| 262 | 1-[3-tert-butyl-1-(4-chloro-3-hydroxyphenyl)-1H-pyrazol-5-yl]-3- | 0.0843 uM |
| | [2-({[3-chloro-1-(2-methoxybenzyl)-6-methyl-2-oxo-1,2-dihydro- | |
| | pyridin-4-yl]oxy}methyl)benzyl]urea | |
| 263 | 1-[3-tert-butyl-1-(4-hydroxyphenyl)-1H-pyrazol-5-yl]-3-[2-({[1- | 0.0231 uM |
| | (2-methoxybenzyl)-3,6-dimethyl-2-oxo-1,2-dihydropyridin-4- | |
| | yl]oxy}methyl)benzyl]urea | |
| 264 | 1-[3-tert-butyl-1-(3-chloro-4-hydroxyphenyl)-1H-pyrazol-5-yl]-3- | 0.0274 uM |
| | [2-({[1-(2-methoxybenzyl)-3,6-dimethyl-2-oxo-1,2-dihydro- | |
| | pyridin-4-yl]oxy}methyl)benzyl]urea | |
| 265 | 1-{3-tert-butyl-1-[3-(2-hydroxyethoxy)phenyl]-1H-pyrazol-5-yl}- | 0.00840 uM |
| | 3-[2-({[3-chloro-1-(3-chloro-4-methoxybenzyl)-6-methyl-2-oxo- | |
| | 1,2-dihydropyridin-4-yl]oxy}methyl)benzyl]urea | |
| 266 | 1-[3-tert-butyl-1-(3-chloro-4-hydroxyphenyl)-1H-pyrazol-5-yl]-3- | 0.0192 uM |
| | [2-({[1-(3-chloro-4-methoxybenzyl)-3,6-dimethyl-2-oxo-1,2- | |
| | dihydropyridin-4-yl]oxy}methyl)benzyl]urea | |
| 267 | 1-[3-tert-butyl-1-(4-hydroxyphenyl)-1H-pyrazol-5-yl]-3-[2-({[1- | 0.0162 uM |
| | (3-chloro-4-methoxybenzyl)-3,6-dimethyl-2-oxo-1,2-dihydro- | |
| | pyridin-4-yl]oxy}methyl)benzyl]urea | |
| 268 | 1-[3-tert-butyl-1-(4-hydroxyphenyl)-1H-pyrazol-5-yl]-3-{2-[({3- | 0.0236 uM |
| | chloro-6-methyl-1-[2-(methylthio)benzyl]-2-oxo-1,2-dihydro- | |
| | pyridin-4-yl}oxy)methyl]benzyl}urea | |
| 269 | 1-{3-tert-butyl-1-[3-(2-hydroxyethoxy)phenyl]-1H-pyrazol-5-yl}- | 0.0204 uM |
| | 3-{2-[({3-chloro-6-methyl-1-[2-(methylthio)benzyl]-2-oxo-1,2- | |
| | dihydropyridin-4-yl}oxy)methyl]benzyl}urea | |
| 270 | 1-[3-tert-butyl-1-(3-chloro-4-hydroxyphenyl)-1H-pyrazol-5-yl]-3- | 0.0327 uM |
| | {2-[({3-chloro-6-methyl-1-[2-(methylthio)benzyl]-2-oxo-1,2- | |
| | dihydropyridin-4-yl}oxy)methyl]benzyl}urea | |
| 271 | 1-[3-tert-butyl-1-(3-hydroxyphenyl)-1H-pyrazol-5-yl]-3-{2-[({3- | 0.0141 uM |
| | chloro-6-methyl-1-[2-(methylthio)benzyl]-2-oxo-1,2- | |
| | dihydropyridin-4-yl}oxy)methyl]benzyl}urea | |
| 272 | N-[4-({4-[(2-{[({[3-tert-butyl-1-(4-chloro-3-hydroxyphenyl)-1H- | 0.00240 uM |
| | pyrazol-5-yl]amino}carbonyl)amino]methyl}benzyl)oxy]-3- | |
| | chloro-6-methyl-2-oxopyridin-1(2H)-yl}methyl)benzyl]- | |
| | glycinamide | |
| 273 | 1-[3-tert-butyl-1-(4-chloro-3-hydroxyphenyl)-1H-pyrazol-5-yl]-3- | 0.121 uM |
| | {2-[({3-chloro-6-methyl-1-[2-(methylthio)benzyl]-2-oxo-1,2- | |
| | dihydropyridin-4-yl}oxy)methyl]benzyl}urea | |
| 274 | N-[4-({4-[(2-{[({[3-tert-butyl-1-(3-chloro-4-hydroxyphenyl)-1H- | 0.00360 uM |
| | pyrazol-5-yl]amino}carbonyl)amino]methyl}benzyl)oxy]-3- | |
| | chloro-6-methyl-2-oxopyridin-1(2H)-yl}methyl)benzyl]- | |
| | glycinamide | |
| 275 | 1-[3-tert-butyl-1-(4-chloro-3-hydroxyphenyl)-1H-pyrazol-5-yl]-3- | 0.0537 uM |
| | [2-({[1-(2-methoxybenzyl)-3,6-dimethyl-2-oxo-1,2- | |
| | dihydropyridin-4-yl]oxy}methyl)benzyl]urea | |
| 276 | 1-[2-({[3-chloro-1-(2-methoxybenzyl)-6-methyl-2-oxo-1,2- | 0.0205 uM |
| | dihydropyridin-4-yl]oxy}methyl)benzyl]-3-{1-(3- | |
| | hydroxyphenyl)-3-[1-methyl-1-(methylthio)ethyl]-1H-pyrazol-5- | |
| | yl}ure | |
| 277 | 1-[3-tert-butyl-1-(4-chloro-3-hydroxyphenyl)-1H-pyrazol-5-yl]-3- | 0.131 uM |
| | {2-[({3,6-dimethyl-1-[2-(methylthio)benzyl]-2-oxo-1,2- | |
| | dihydropyridin-4-yl}oxy)methyl]benzyl}urea | |
| 278 | 1-[3-tert-butyl-1-(3-hydroxyphenyl)-1H-pyrazol-5-yl]-3-{2- | 0.0132 uM |
| | [({3,6-dimethyl-1-[2-(methylthio)benzyl]-2-oxo-1,2- | |
| | dihydropyridin-4-yl}oxy)methyl]benzyl}urea | |
| 279 | 3-{3-bromo-4-[(2-{[({[3-tert-butyl-1-(4-methylphenyl)-1H- | 0.00367 uM |
| | pyrazol-5-yl]amino}carbonyl)-amino]methyl}-4- | (0.000652-0.0206 |
| | fluorobenzyl)oxy]-6-methyl-2-oxopyridin-1(2H)-yl}-N,4- | n = 2) |
| | dimethylbenzamide | |

The above detailed description of embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

1. A compound of Formula I: 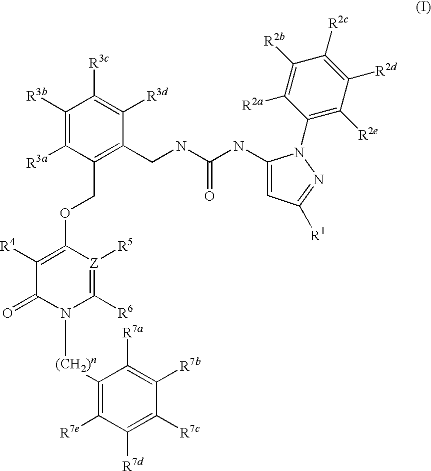
or a pharmaceutically acceptable salt thereof, wherein;
Z is C or N;
n is 0 or 1;
R¹ is (C₁-C₄)-alkyl, (C₃-C₆)-cycloalkyl, or [(C₁-C₄)-alkyl)][(C₁-C₄)-alkyl-S]—(C₁-C₄)-alkyl; 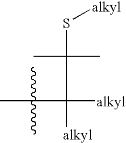
R^{2a}, R^{2b}, R^{2c}, R^{2d} and R^{2e} are independently —H, (C₁-C₄)-alkyl, —OH, halo, (C₁-C₄)-alkyl-O—, heterocyclyloxy-(C₁-C₄)-alkyl-O—, OH—(C₁-C₄)-alkyl-O—, or (C₁-C₄)-alkyl[(C₁-C₄)-dialkyl]silyloxy;
R^{3a}, R^{3b}, R^{3c} and R^{3d} are independently —H or halo;
R⁴ is —H, halo or (C₁-C₄)-alkyl;
R⁵ is —H or absent when Z is N;
R⁶ is —H, (C₁-C₄)-alkyl or (C₁-C₄)-alkyl-S—; and
R^{7a}, R^{7b}, R^{7c}, R^{7d} and R^{7e} are independently —H, —C(O)—O—(C₁-C₄)-alkyl, (C₁-C₄)-alkyl-S—, (C₁-C₄)-alkyl-O—, —OH, NH₂—(C₁-C₄)-alkyl-C(O)—NH—(C₁-C₄)-alkyl-,
(C₁-C₄)-alkyl-O—C(O)—NH—
—(C₁-C₄)-alkyl-C(O)—NH—(C₁-C₄)-alkyl,
halo, —CN 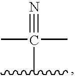
OH—(C₁-C₄)—(C₁-C₄)-alkyl-C(O)—NH—(C₁-C₄)-alkyl-,
(C₁-C₄)-alkyl,
—C(O)—OH,
OH—(C₁-C₄)-alkyl-NH—C(O)—,
(C₁-C₄)-alkyl-NH—C(O)—,
(OH)₂—(C₁-C₄)-alkyl-NH—C(O)—,
(C₁-C₄)-alkyl-O—(C₁-C₄)-alkyl-NH—C(O)—,
NH₂—C(O)—(C₁-C₄)-alkyl-NH—C(O)—,
(C₁-C₄)-alkyl-NH—C(O)—(C₁-C₄)-alkyl-NH—C(O)—,
(C₁-C₄)-dialkyl-N—(C₁-C₄)-alkyl-NH—C(O)—,
or [N₂—C(O)][(C₁-C₄)-alkyl](C₁-C₄)-alkyl-N₂—C(O)—.

2. The compound of claim 1 or a pharmaceutically acceptable salt thereof wherein:
Z is C or N;
n is 1;
R¹ is (C₁-C₄)-alkyl, (C₃-C₆)-cycloalkyl, or [(C₁-C₄)-alkyl)][(C₁-C₄)-alkyl-S]—(C₁-C₄)-alkyl;
R^{2a}, R^{2b}, R^{2c}, R^{2d} and R^{2e} are independently —H, (C₁-C₄)-alkyl, —OH, halo, (C₁-C₄)-alkyl-O—, heterocyclyloxy-(C₁-C₄)-alkyl-O—, OH—(C₁-C₄)-alkyl-O—, or (C₁-C₄)-alkyl[(C₁-C₄)-dialkyl]silyloxy;
R^{3a}, R^{3b}, R^{3c} and R^{3d} are independently —H or halo;
R⁴ is —H, halo or (C₁-C₄)-alkyl;
R⁵ is —H or absent when Z is N;
R⁶ is —H or (C₁-C₄)-alkyl; and
R^{7a}, R^{7b}, R^{7c}, R^{7d} and R^{7e} are independently —H, —C(O)—O—(C₁-C₄)-alkyl, (C₁-C₄)-alkyl-S—, (C₁-C₄)-alkyl-O—, —OH, NH₂—(C₁-C₄)-alkyl-C(O)—NH—(C₁-C₄)-alkyl, (C₁-C₄)-alkyl-O—C(O)—NH—(C₁-C₄)-alkyl-C(O)—NH—(C₁-C₄)-alkyl, —CN, halo 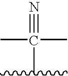
or OH—(C₁-C₄)-alkyl-C(O)—NH—(C₁-C₄)-alkyl.

3. The compound of claim 2 or a pharmaceutically acceptable salt thereof wherein:
Z is —C;
R¹ is (C₁-C₄)-alkyl, (C₃-C₆)-cycloalkyl, or [(C₁-C₄)-alkyl)][(C₁-C₄)-alkyl-S]—(C₁-C₄)-alkyl;
R^{2a}, R^{2b}, R^{2c}, R^{2d} and R^{2e} are independently —H, (C₁-C₄)-alkyl, —OH, halo, (C₁-C₄)-alkyl-O—, heterocyclyloxy-(C₁-C₄)-alkyl-O—, OH—(C₁-C₄)-alkyl-O—, or (C₁-C₄)-alkyl[(C₁-C₄)-dialkyl]silyloxy;
R^{3a}, R^{3b}, R^{3c} and R^{3d} are independently —H or halo;
R⁴ is —H, halo or (C₁-C₄)-alkyl;
R⁵ is —H;
R⁶ is (C₁-C₄)-alkyl; and
R^{7a}, R^{7b}, R^{7c}, R^{7d} and R^{7e} are independently —H, —C(O)—O—(C₁-C₄)-alkyl (C₁-C₄)-alkyl-S—, (C₁-C₄)-alkyl-O—, —OH, NH₂—(C₁-C₄)-alkyl-C(O)—NH—(C₁-C₄)-alkyl, (C₁-C₄)-alkyl-O—C(O)—NH—(C₁-C₄)-alkyl-C(O)—NH—(C₁-C₄)-alkyl-, (C₁-C₄)-alkyl-O—C(O)₂—NH—(C₁-C₄)-alkyl-C(O)₂—NH—(C₁-C₄)-alkyl-, halo, 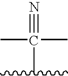
or OH—(C₁-C₄)-alkyl-C(O)—NH—(C₁-C₄)-alkyl.

4. The compound of claim 2 or a pharmaceutically acceptable salt thereof wherein:
Z is N;
R¹ is (C₁-C₄)-alkyl;
R^{2a}, R^{2b}, R^{2c}, R^{2d} and R^{2e} are independently —H, (C₁-C₄)-alkyl, halo or (C₁-C₄)-alkyl-O—;
R^{3a}, R^{3b}, R^{3c} and R^{3d} are —H;
R⁴ is (C₁-C₄)-alkyl;
R⁵ is absent when Z is N;
R⁶ is —H; and
R^{7a}, R^{7b}, R^{7c}, R^{7d} and R^{7e} are independently —H or (C₁-C₄)-alkyl-O—.

5. The compound of claim 1 or a pharmaceutically acceptable salt thereof wherein:
Z is C or N;
n is 0;
R¹ is (C₁-C₄)-alkyl;
R^{2a}, R^{2b}, R^{2c}, R^{2d} and R^{2e} are independently H, (C₁-C₄)-alkyl, —OH, halo, (C₁-C₄)-alkyl-O— or OH—(C₁-C₄)-alkyl-O—;
R^{3a}, R^{3b}, R^{3c} and R^{3d} are independently —H or halo;
R⁴ is —H or halo;
R⁵ is —H or absent when Z is N;
R⁶ is (C₁-C₄)-alkyl or (C₁-C₄)-alkyl-S—; and
R^{7a}, R^{7b}, R^{7c}, R^{7d} and R^{7e} are independently —H, —C(O)—O—(C₁-C₄)-alkyl (C₁-C₄)-alkyl, —C(O)—OH, OH—(C₁-C₄)-alkyl-NH—C(O)—, OH—(C₁-C₄)-alkyl-NH—C(O)₂H—, (C₁-C₄)-alkyl-NH—C(O)—, (OH)₂—(C₁-C₄)-alkyl-NH—C(O)—, (OH)₂—[(C₁-C₄)-alkyl]-NH—C(O)₂H, (C₁-C₄)-alkyl-O—(C₁-C₄)-alkyl-NH—C(O)—, (C₁-C₄)-alkyl-O—(C₁-C₄)-alkyl-NH—C(O)—, NH₂—C(O)—C₁-C₄)-alkyl-NH—C(O)—, (C₁-C₄)-alkyl-NH—C(O)—O—(C₁-C₄)-alkyl-NH—C(O)—OH—, (C₁-C₄)-dialkyl-NH—(C₁-C₄)-alkyl-NH—C(O)—, or [NH₂—C(O)][(C₁-C₄)-alkyl](C₁-C₄)-alkyl-NH—C(O)—.

6. The compound of claim 5 or a pharmaceutically acceptable salt thereof wherein:
Z is —C;
R¹ is —(C₁-C₄)-alkyl;
R^{2a}, R^{2b}, R^{2c}, R^{2d} and R^{2e} are independently —H, (C₁-C₄)-alkyl, —OH, halo, (C₁-C₄)-alkyl-O— or OH—(C₁-C₄)-alkyl-O—;
R^{3a}, R^{3b}, R^{3c} and R^{3d} are independently —H or -halo;
R⁴ is —H or -halo;
R⁵ is —H;
R⁶ is —(C₁-C₄)-alkyl or (C₁-C₄)-alkyl-S—; and
R^{7a}, R^{7b}, R^{7c}, R^{7d} and R^{7e} are independently —H, —C(O)—O—(C₁-C₄)-alkyl, —(C₁-C₄)-alkyl, —C(O)—OH, OH—(C₁-C₄)-alkyl-NH—C(O)—, (C₁-C₄)-alkyl-NH—C(O)—, (OH)₂—(C₁-C₄)-alkyl-NH—C(O)—, (C₁-C₄)-alkyl-O—(C₁-C₄)-alkyl-NH—C(O)—, NH₂—C(O)—(C₁-C₄)-alkyl-NH—C(O)—, (C₁-C₄)-alkyl-NH—C(O)—(C₁-C₄)-alkyl-NH—C(O)—, (C₁-C₄)-dialkyl-N—(C₁-C₄)-alkyl-NH—C(O)—, or [NH₂—C(O)][(C₁-C₄)-alkyl](C₁-C₄)-alkyl-NH—C(O)—.

7. The compound of claim 5 or a pharmaceutically acceptable salt thereof wherein:
Z is —N;
R¹ is (C₁-C₄)-alkyl;
R^{2a}, R^{2b}, R^{2c}, R^{2d} and R^{2e} are independently —H or (C₁-C₄)-alkyl;
R^{3a}, R^{3b}, R^{3c} and R^{3d} are independently —H or halo;
R⁴ is —H or halo;
R⁵ is absent;
R⁶ is (C₁-C₄)-alkyl-S—; and
R^{7a}, R^{7b}, R^{7c}, R^{7d} and R^{7e} are independently —H, (C₁-C₄)-alkyl-O—C(O)—, or (C₁-C₄)-alkyl.

8. The compound of claim 1 or a pharmaceutically acceptable salt thereof wherein:
Z is —C or —N;
n is 0 or 1;
R¹ is —C—(CH₃)₃, 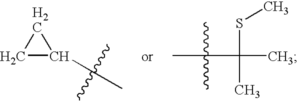
R^{2a}, R^{2b}, R^{2c}, R^{2d} and R^{2e} are independently —H, —Cl, —CH₃, OH—, —O—CH₃, OH—CH₂—CH₂—O—, —F, difluoropyranyloxyethoxy or tert-butyl[dimethyl]silyloxy;
R^{3a}, R^{3b}, R^{3c} and R^{3d} are independently —H or —F;
R⁴ is —H, —Br, —Cl, —CH₃ or —CH₂—CH₃;
R⁵ is —H or is absent when Z is —N;
R⁶ is —H, —CH₃ or CH₃—S—; and
R^{7a}, R^{7b}, R^{7c}, R^{7d} and R^{7e} are independently —H, —C(O)—O—CH₃, CH₃—S—, —O—CH₃, —OH, NH₂—CH₂—C(O)—NH—CH₂—,
tert-butoxy-C(O)—NH—CH₂—C(O)—NH—CH₂—, Cl, OH—CH₂—C(O)—NH—CH₂—, —CH₃, —C(O)—OH, —C(O)—NH₂, (OH)₂—(CH₂)₃—NH—C(O)—, NH₂—C(O)—CH₂—NH₂—C(O)—,

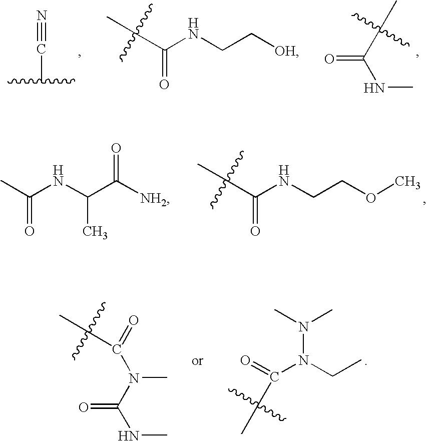

9. The compound of claim 2 or a pharmaceutically acceptable salt thereof wherein:
Z is —C or —N;
R¹ is —C—(CH₃)₃, 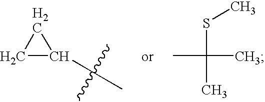
R^{2a}, R^{2b}, R^{2c}, R^{2d} and R^{2e} are independently —H, —Cl, —CH₃, OH—, —O—CH₃, OH—CH₂—CH₂—O—, —F, difluoropyranyloxyethoxy or 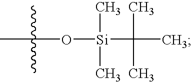
R^{3a}, R^{3b}, R^{3c} and R^{3d} are independently —H or —F;
R⁴ is —H, —Br, —Cl, —CH₃ or —CH₂—CH₃;
R⁵ is —H or is absent when Z is N;
R⁶ is —H or —CH₃ and
R^{7a}, R^{7b}, R^{7c}, R^{7d} and R^{7e} are independently —H, —C(O)—O—CH₃, CH₃—S—, —O—CH₃, —OH, NH₂—CH₂—C(O)—NH—CH₂—, 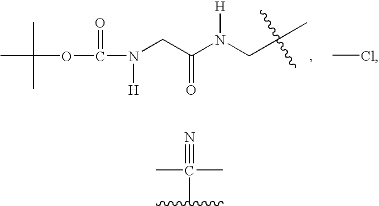
or. OH—CH2-C(O)—NH—CH2-

10. A compound of claim 3 or a Pharmaceutically acceptable salt thereof wherein:
R¹ is —C—(CH₃)₃, 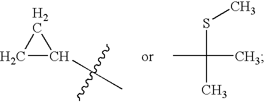
R^{2a}, R^{2b}, R^{2c}, R^{2d} and R^{2e} are independently —H, —Cl, —CH₃, —OH, —O—CH₃, —OH—CH₂—CH₂—O—, —F, difluoropyranyloxyethoxy or 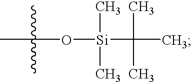
R^{3a}, R^{3b}, R^{3c} and R^{3d} are independently —H or —F;
R⁴ is —H, —Br, —Cl or —CH₃;
R⁵ is —H;
R⁶ is —CH₃; and
R^{7a}, R^{7b}, R^{7c}, R^{7d} and R^{7e} are independently H, —C(O)—O—CH₃, CH₃—S—, —O—CH₃, —OH, CH₃—NH—C(O)—CH₂—NH—C(O)—, NH₂—CH₂—C(O)—NH—CH₂—, 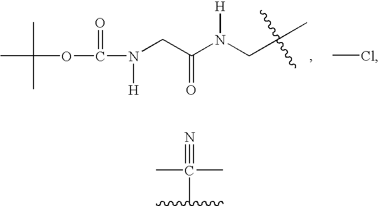
or OH—CH2-C(O)—NH—CH2-.

11. The compound of claim 4 or a pharmaceutically acceptable salt thereof wherein:
R¹ is —C—(CH₃)₃; —CH₂—CH(CH₃)₂;
R^{2a}, R^{2b}, R^{2c}, R^{2d} and R^{2e} are independently —H, —CH₃, —F or —O—CH₃;
R^{3a}, R^{3b}, R^{3c} and R^{3d} are —H;
R⁴ is —CH₂—CH₃;
R⁵ is —H;
R⁶ is —H; and
R^{7a}, R^{7b}, R^{7c}, R^{7d} and R^{7e} are independently —H or —O—CH₃.

12. The compound of claim 5 or a pharmaceutically acceptable salt thereof wherein:
Z is —C or —N;
R¹ is —C—(CH₃)₃;
R^{2a}, R^{2b}, R^{2c}, R^{2d} and R^{2e} are independently —H, —Cl, —CH₃, —OH, —O—CH₃ or OH—CH₂—CH₂—O—,
R^{3a}, R^{3b}, R^{3c} and R^{3d} are independently —H or —F;
R⁴ is —H, —Br or —Cl;
R⁵ is —H or absent when Z is —N;
R⁶ is —CH₃ or CH₃—S—; and
R^{7a}, R^{7b}, R^{7c}, R^{7d} and R^{7e} are independently H, —C(O)—O—CH₃, CH₃, —C(O)—OH, 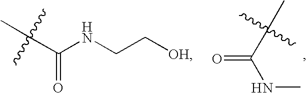
(OH)₂—(CH₂)₃—NH—C(O)—, 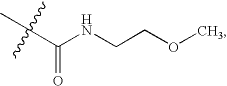
NH₂—C(O)—CH₂—NH₂—C(O)—, 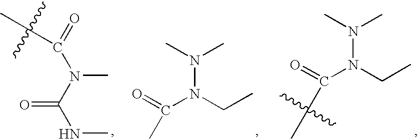
—C(O)—N(N—CH₃)₂—CH₂—CH₃, or

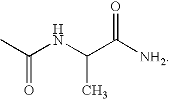

13. The compound of claim 6 or a pharmaceutically acceptable salt thereof wherein:
R¹ is —C—(CH₃)₃;
R^{2a}, R^{2b}, R^{2c}, R^{2d} and R^{2e} are independently —H, —Cl, —CH₃, —OH, —O—CH₃ or OH—CH₂—CH₂—O—,
R^{3a}, R^{3b}, R^{3c} and R^{3d} are independently —H or —F;
R⁴ is —H, —Br or —Cl;
R⁵ is —H;
R⁶ is —CH₃ or CH₃—S—; and
R^{7a}, R^{7b}, R^{7c}, R^{7d} and R^{7e} are independently —H, —C(O)—O—CH₃, —CH₃, —C(O)—OH, 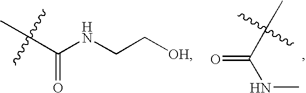
(OH)₂—(CH₂)₃—NH—C(O)—, 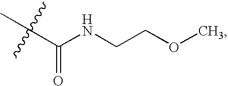
NH₂—C(O)—CH₂—NH₂—C(O)—

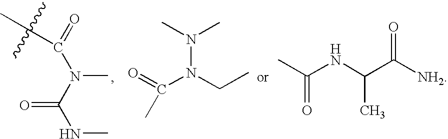

14. A method of treating asthma in a mammal, said method comprising administering to said mammal an effective amount of a compound of claim 1 or a pharmaceutically acceptable salt thereof.

15. A compound of formula I of claim 1 or a pharmaceutically acceptable salt thereof selected from the group consisting of:
1-[2-({[3-bromo-1-(2,4-dimethoxybenzyl)-6-methyl-2-oxo-1,2-dihydropyridin-4-yl]oxy}methyl)benzyl]-3-[3-tert-butyl-1-(3-chlorophenyl)-1H-pyrazol-5-yl]urea;
1-[2-({[3-bromo-1-(2,4-dimethoxybenzyl)-6-methyl-2-oxo-1,2-dihydropyridin-4-yl]oxy}methyl)benzyl]-3-[3-tert-butyl-1-(3-methoxyphenyl)-1H-pyrazol-5-yl]urea;
1-[2-({[3-bromo-1-(2,4-dimethoxybenzyl)-6-methyl-2-oxo-1,2-dihydropyridin-4-yl]oxy}methyl)benzyl]-3-(3-tert-butyl-1-phenyl-1H-pyrazol-5-yl)urea;
1-[2-({[3-bromo-1-(2,4-dimethoxybenzyl)-6-methyl-2-oxo-1,2-dihydropyridin-4-yl]oxy}methyl)benzyl]-3-[3-tert-butyl-1-(2,4-difluorophenyl)-1H-pyrazol-5-yl]urea;
1-[2-({[3-bromo-1-(2,4-dimethoxybenzyl)-6-methyl-2-oxo-1,2-dihydropyridin-4-yl]oxy}methyl)benzyl]-3-[3-tert-butyl-1-(4-methylphenyl)-1H-pyrazol-5-yl]urea;
1-(3-tert-butyl-1-{3-[2-(tetrahydro-2H-pyran-2-yloxy)ethoxy]phenyl}-1H-pyrazol-5-yl)-3-[5-fluoro-2-({[(3-methoxybenzyl)-6-methyl-2-oxo-1,2-dihydropyridin-4-yl]oxy}-methyl)benzyl]urea;
1-[3-tert-butyl-1-(3-methoxyphenyl)-1H-pyrazol-5-yl]-3-[5-fluoro-2-({[1-(3-methoxybenzyl)-6-methyl-2-oxo-1,2-dihydropyridin-4-yl]oxy}methyl)benzyl]urea;
1-(3-tert-butyl-1-{3-[2-(tetrahydro-2H-pyran-2-yloxy)ethoxy]phenyl}-1H-pyrazol-5-yl)-3-[2-({[1-(3-methoxybenzyl)-6-methyl-2-oxo-1,2-dihydropyridin-4-yl]oxy}methyl)-benzyl]urea;
1-{3-tert-butyl-1-[3-(2-hydroxyethoxy)phenyl]-1H-pyrazol-5-yl}-3-[5-fluoro-2-({[1-(3-methoxybenzyl)-6-methyl-2-oxo-1,2-dihydropyridin-4-yl]oxy}methyl)benzyl]urea;
1-{3-tert-butyl-1-[3-(2-hydroxyethoxy)phenyl]-1H-pyrazol-5-yl}-3-[2-({[1-(3-methoxy-benzyl)-6-methyl-2-oxo-1,2-dihydropyridin-4-yl]oxy}methyl)benzyl]urea;
1-{3-tert-butyl-1-[3-(2-hydroxyethoxy)phenyl]-1H-pyrazol-5-yl}-3-[2-({[3-chloro-1-(3-methoxybenzyl)-6-methyl-2-oxo-1,2-dihydropyridin-4-yl]oxy}methyl)-5-fluorobenzyl]-urea;
1-(3-tert-butyl-1-{3-[2-(tetrahydro-2H-pyran-2-yloxy)ethoxy]phenyl}-1H-pyrazol-5-yl)-3-[2-({[3-chloro-1-(3-methoxybenzyl)-6-methyl-2-oxo-1,2-dihydropyridin-4-yl]oxy}-methyl)-5-fluorobenzyl]urea;
1-[3-tert-butyl-1-(4-{[tert-butyl(dimethyl)silyl]oxy}-3-chlorophenyl)-1H-pyrazol-5-yl]-3-[2-({[3-chloro-1-(3-methoxybenzyl)-6-methyl-2-oxo-1,2-dihydropyridin-4-yl]oxy}-methyl)-5-fluorobenzyl]urea;
1-[3-tert-butyl-1-(3-{[tert-butyl(dimethyl)silyl]oxy}-4-chlorophenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluorobenzyl]urea;
1-[3-tert-butyl-1-(3-methoxyphenyl)-1H-pyrazol-5-yl]-3-[2-({[3-chloro-1-(3-methoxybenzyl)-6-methyl-2-oxo-1,2-dihydropyridin-4-yl]oxy}methyl)benzyl]urea;
1-[3-tert-butyl-1-(4-chloro-3-hydroxyphenyl)-1H-pyrazol-5-yl]-3-[2-({[3-chloro-1-(3-methoxybenzyl)-6-methyl-2-oxo-1,2-dihydropyridin-4-yl]oxy}methyl)-5-fluoro-benzyl]urea;
1-{3-tert-butyl-1-[3-(2-hydroxyethoxy)phenyl]-1H-pyrazol-5-yl}-3-[2-({[1-(3-methoxy-benzyl)-3,6-dimethyl-2-oxo-1,2-dihydropyridin-4-yl]oxy}methyl)benzyl]urea;
1-[3-tert-butyl-1-(3-hydroxyphenyl)-1H-pyrazol-5-yl]-3-[2-({[3-chloro-1-(3-methoxy-benzyl)-6-methyl-2-oxo-1,2-dihydropyridin-4-yl]oxy}methyl)-5-fluoro-benzyl]urea;
1-[3-tert-butyl-1-(4-hydroxyphenyl)-1H-pyrazol-5-yl]-3-[2-({[3-chloro-1-(3-methoxy-benzyl)-6-methyl-2-oxo-1,2-dihydropyridin-4-yl]oxy}methyl)benzyl]-urea;
1-[3-tert-butyl-1-(3-hydroxyphenyl)-1H-pyrazol-5-yl]-3-[2-({[3-chloro-1-(3-methoxy-benzyl)-6-methyl-2-oxo-1,2-dihydropyridin-4-yl]oxy}methyl)-benzyl]urea;
1-[3-tert-butyl-1-(4-hydroxyphenyl)-1H-pyrazol-5-yl]-3-[2-({[1-(4-methoxybenzyl)-6-methyl-2-oxo-1,2-dihydropyridin-4-yl]oxy}methyl)benzyl]urea;
1-[3-tert-butyl-1-(3-hydroxyphenyl)-1H-pyrazol-5-yl]-3-[2-({[1-(4-methoxybenzyl)-6-methyl-2-oxo-1,2-dihydropyridin-4-yl]oxy}methyl)benzyl]urea;
1-[3-tert-butyl-1-(3-chloro-4-hydroxyphenyl)-1H-pyrazol-5-yl]-3-[2-({[1-(4-methoxy-benzyl)-6-methyl-2-oxo-1,2-dihydropyridin-4-yl]oxy}methyl)benzyl]urea;
1-{3-tert-butyl-1-[3-(2-hydroxyethoxy)phenyl]-1H-pyrazol-5-yl}-3-[2-({[3-chloro-1-(4-methoxybenzyl)-6-methyl-2-oxo-1,2-dihydropyridin-4-yl]oxy}methyl)-benzyl]urea;
1-[3-tert-butyl-1-(3-chloro-4-hydroxyphenyl)-1H-pyrazol-5-yl]-3-[2-({[3-chloro-1-(4-methoxybenzyl)-6-methyl-2-oxo-1,2-dihydropyridin-4-yl]oxy}methyl)benzyl]urea;
methyl 3-{4-[(2-{[({[3-tert-butyl-1-(4-methylphenyl)-1H-pyrazol-5-yl]amino}-carbonyl)-amino]methyl}-4-fluorobenzyl)oxy]-2-(methylthio)-6-oxopyrimidin-1(6H)-yl}-4-methyl-benzoate;
1-{3-tert-butyl-1-[3-(2-hydroxyethoxy)phenyl]-1H-pyrazol-5-yl}-3-[2-({[3-chloro-1-(4-methoxybenzyl)-6-methyl-2-oxo-1,2-dihydropyridin-4-yl]oxy}methyl)-5-fluorobenzyl]-urea;
1-[3-tert-butyl-1-(3-chloro-4-hydroxyphenyl)-1H-pyrazol-5-yl]-3-[2-({[3-chloro-1-(4-methoxybenzyl)-6-methyl-2-oxo-1,2-dihydropyridin-4-yl]oxy}methyl)-5-fluorobenzyl]-urea;
1-[3-tert-butyl-1-(4-hydroxyphenyl)-1H-pyrazol-5-yl]-3-[2-({[3-chloro-1-(3-chloro-4-methoxybenzyl)-6-methyl-2-oxo-1,2-dihydropyridin-4-yl]oxy}methyl)-benzyl]urea;
1-[3-tert-butyl-1-(3-hydroxyphenyl)-1H-pyrazol-5-yl]-3-[2-({[3-chloro-1-(4-chloro-3-methoxybenzyl)-6-methyl-2-oxo-1,2-dihydropyridin-4-yl]oxy}methyl)-benzyl]urea;
1-[2-({[3-bromo-1-(4-methoxybenzyl)-6-methyl-2-oxo-1,2-dihydropyridin-4-yl]oxy}-methyl)benzyl]-3-[3-tert-butyl-1-(3-fluorophenyl)-1H-pyrazol-5-yl]urea;
1-[2-({[3-bromo-1-(4-methoxybenzyl)-6-methyl-2-oxo-1,2-dihydropyridin-4-yl]oxy}-methyl)benzyl]-3-[3-tert-butyl-1-(3-chlorophenyl)-1H-pyrazol-5-yl]urea;
1-[2-({[3-bromo-1-(4-methoxybenzyl)-6-methyl-2-oxo-1,2-dihydropyridin-4-yl]oxy}-methyl)benzyl]-3-[3-tert-butyl-1-(3-methoxyphenyl)-1H-pyrazol-5-yl]urea;
1-[2-({[3-bromo-1-(4-hydroxybenzyl)-6-methyl-2-oxo-1,2-dihydropyridin-4-yl]oxy}-methyl)benzyl]-3-[3-tert-butyl-1-(4-methylphenyl)-1H-pyrazol-5-yl]urea;
1-[2-({[3-bromo-1-(4-methoxybenzyl)-6-methyl-2-oxo-1,2-dihydropyridin-4-yl]oxy}-methyl)benzyl]-3-[3-tert-butyl-1-(4-methylphenyl)-1H-pyrazol-5-yl]urea;
1-[2-({[3-bromo-1-(4-methoxybenzyl)-6-methyl-2-oxo-1,2-dihydropyridin-4-yl]oxy}-methyl)benzyl]-3-(3-cyclopropyl-1-phenyl-1H-pyrazol-5-yl)urea;
1-(2-{[(1-benzyl-3-bromo-6-methyl-2-oxo-1,2-dihydropyridin-4-yl)oxy]methyl}-benzyl)-3-[3-tert-butyl-1-(3-fluorophenyl)-1H-pyrazol-5-yl]urea;
1-(2-{[(1-benzyl-3-bromo-6-methyl-2-oxo-1,2-dihydropyridin-4-yl)oxy]methyl}-benzyl)-3-(3-tert-butyl-1-phenyl-1H-pyrazol-5-yl)urea;
1-(2-{[(1-benzyl-3-bromo-6-methyl-2-oxo-1,2-dihydropyridin-4-yl)oxy]methyl}-benzyl)-3-[3-tert-butyl-1-(2,4-difluorophenyl)-1H-pyrazol-5-yl]urea;
1-(2-{[(1-benzyl-3-bromo-6-methyl-2-oxo-1,2-dihydropyridin-4-yl)oxy]methyl}-benzyl)-3-[3-tert-butyl-1-(4-methylphenyl)-1H-pyrazol-5-yl]urea;
1-(2-{[(1-benzyl-3-bromo-6-methyl-2-oxo-1,2-dihydropyridin-4-yl)oxy]methyl}-benzyl)-3-(3-cyclopropyl-1-phenyl-1H-pyrazol-5-yl)urea;
1-(2-{[(1-benzyl-3-bromo-6-methyl-2-oxo-1,2-dihydropyridin-4-yl)oxy]methyl}benzyl)-3-[3-tert-butyl-1-(3-chlorophenyl)-1H-pyrazol-5-yl]urea;
1-(2-{[(1-benzyl-3-bromo-6-methyl-2-oxo-1,2-dihydropyridin-4-yl)oxy]methyl}benzyl)-3-[3-tert-butyl-1-(3-methoxyphenyl)-1H-pyrazol-5-yl]urea;
1-(2-{[(1-benzyl-5-ethyl-6-oxo-1,6-dihydropyrimidin-4-yl)oxy]methyl}benzyl)-3-[3-tert-butyl-1-(4-methylphenyl)-1H-pyrazol-5-yl]urea;
1-(2-{[(1-benzyl-5-ethyl-6-oxo-1,6-dihydropyrimidin-4-yl)oxy]methyl}benzyl)-3-[3-tert-butyl-1-(3-fluorophenyl)-1H-pyrazol-5-yl]urea;
1-(2-{[(1-benzyl-5-ethyl-6-oxo-1,6-dihydropyrimidin-4-yl)oxy]methyl}benzyl)-3-(3-tert-butyl-1-phenyl-1H-pyrazol-5-yl)urea;
1-[3-tert-butyl-1-(4-methylphenyl)-1H-pyrazol-5-yl]-3-[2-({[5-ethyl-1-(4-methoxy-benzyl)-6-oxo-1,6-dihydropyrimidin-4-yl]oxy}methyl)benzyl]urea;
1-[3-tert-butyl-1-(3-methoxyphenyl)-1H-pyrazol-5-yl]-3-[2-({[5-ethyl-1-(4-methoxy-benzyl)-6-oxo-1,6-dihydropyrimidin-4-yl]oxy}methyl)benzyl]urea;
1-[3-tert-butyl-1-(3-fluorophenyl)-1H-pyrazol-5-yl]-3-[2-({[5-ethyl-1-(4-methoxy-benzyl)-6-oxo-1,6-dihydropyrimidin-4-yl]oxy}methyl)benzyl]urea;
1-[2-({[3-bromo-1-(2,4-dimethoxybenzyl)-6-methyl-2-oxo-1,2-dihydropyridin-4-yl]-oxy}methyl)benzyl]-3-[3-tert-butyl-1-(3-fluorophenyl)-1H-pyrazol-5-yl]urea;
1-[2-({[3-bromo-1-(2,4-dimethoxybenzyl)-6-methyl-2-oxo-1,2-dihydropyridin-4-yl]-oxy}methyl)benzyl]-3-[3-tert-butyl-1-(3-methoxyphenyl)-1H-pyrazol-5-yl]urea;
1-[2-({[3-bromo-1-(2,4-dimethoxybenzyl)-6-methyl-2-oxo-1,2-dihydropyridin-4-yl]-oxy}methyl)benzyl]-3-(3-tert-butyl-1-phenyl-1H-pyrazol-5-yl)urea;
1-[2-({[3-bromo-1-(2,4-dimethoxybenzyl)-6-methyl-2-oxo-1,2-dihydropyridin-4-yl]-oxy}methyl)benzyl]-3-[3-tert-butyl-1-(2,4-difluorophenyl)-1H-pyrazol-5-yl]urea;
methyl 3-{4-[(2-{[({[3-tert-butyl-1-(4-methylphenyl)-1H-pyrazol-5-yl]amino}-carbonyl)-amino]methyl}-4-fluorobenzyl)oxy]-3-chloro-6-methyl-2-oxopyridin-1(2H)-yl}-4-methylbenzoate;
methyl 3-{4-[(2-{[({[3-tert-butyl-1-(3-chlorophenyl)-1H-pyrazol-5-yl]amino}-carbonyl)-amino]methyl}-4-fluorobenzyl)oxy]-3-chloro-6-methyl-2-oxopyridin-1(2H)-yl}-4-methylbenzoate;
methyl 3-{4-[(2-{[({[3-tert-butyl-1-(3-methoxyphenyl)-1H-pyrazol-5-yl]amino}-carbonyl)amino]methyl}-4-fluorobenzyl)oxy]-3-chloro-6-methyl-2-oxopyridin-1(2H)-yl}-4-methylbenzoate;
methyl 3-{3-bromo-4-[(2-{[({[3-tert-butyl-1-(4-methylphenyl)-1H-pyrazol-5-yl]amino}-carbonyl)amino]methyl}-4-fluorobenzyl)oxy]-6-methyl-2-oxopyridin-1(2H)-yl}-4-methylbenzoate;
3-{4-[(2-{[({[3-tert-butyl-1-(4-methylphenyl)-1H-pyrazol-5-yl]amino}carbonyl)-amino]methyl}-4-fluoro-benzyl)oxy]-3-chloro-6-methyl-2-oxopyridin-1(2H)-yl}-4-methylbenzoic acid;
3-{4-[(2-{[({[3-tert-butyl-1-(3-methoxyphenyl)-1H-pyrazol-5-yl]amino}carbonyl)-amino]methyl}-4-fluoro-benzyl)oxy]-3-chloro-6-methyl-2-oxopyridin-1(2H)-yl}-4-methylbenzoic acid;
3-{4-[(2-{[({[3-tert-butyl-1-(4-methylphenyl)-1H-pyrazol-5-yl]amino}carbonyl)-amino]methyl}-4-fluoro-benzyl)oxy]-3-chloro-6-methyl-2-oxopyridin-1(2H)-yl}-N-(2-hydroxyethyl)-4-methylbenzamide;
3-{3-bromo-4-[(2-{[({[3-tert-butyl-1-(4-methyl-phenyl)-1H-pyrazol-5-yl]amino}-carbonyl)-amino]methyl}-4-fluorobenzyl)oxy]-6-methyl-2-oxopyridin-1(2H)-yl}-N-(2-hydroxyethyl)-4-methyl-benzamide;
3-{4-[(2-{[({[3-tert-butyl-1-(3-chlorophenyl)-1H-pyrazol-5-yl]amino}-carbonyl)amino]-methyl}-4-fluorobenzyl)oxy]-3-chloro-6-methyl-2-oxopyridin-1(2H)-yl}-N,4-dimethyl-benzamide;
3-{4-[(2-{[({[3-tert-butyl-1-(3-chlorophenyl)-1H-pyrazol-5-yl]amino}-carbonyl)amino]-methyl}-4-fluorobenzyl)oxy]-3-chloro-6-methyl-2-oxopyridin-1(2H)-yl}-N-(2-hydroxy-ethyl)-4-methylbenzamide;
1-[3-tert-butyl-1-(3-methoxyphenyl)-1H-pyrazol-5-yl]-3-[2-({[3-chloro-1-(3-methoxy-benzyl)-6-methyl-2-oxo-1,2-dihydropyridin-4-yl]oxy}methyl)-5-fluoro-benzyl]urea;
1-[3-tert-butyl-1-(4-chloro-3-hydroxyphenyl)-1H-pyrazol-5-yl]-3-[2-({[3-chloro-1-(4-methoxy-benzyl)-6-methyl-2-oxo-1,2-dihydropyridin-4-yl]oxy}methyl)-5-fluoro-benzyl]-urea;
methyl 3-{4-[(2-{[({[3-tert-butyl-1-(3-hydroxy-phenyl)-1H-pyrazol-5-yl]amino}-carbonyl)amino]-methyl}-4-fluorobenzyl)oxy]-3-chloro-6-methyl-2-oxopyridin-1(2H)-yl}-4-methylbenzoate;
methyl 3-{4-[(2-{[({[3-tert-butyl-1-(4-methylphenyl)-1H-pyrazol-5-yl]amino}-carbonyl)-amino]-methyl}-benzyl)oxy]-2-(methylthio)-6-oxopyrimidin-1(6H)-yl}-4-methyl-benzoate;
methyl 3-{4-[(2-{[({[3-tert-butyl-1-(4-methylphenyl)-1H-pyrazol-5-yl]amino}-carbonyl)-amino]methyl}benzyl)oxy]-3-chloro-6-methyl-2-oxopyridin-1(2H)-yl}-4-methyl-benzoate;
3-{4-[(2-{[({[3-tert-butyl-1-(4-methylphenyl)-1H-pyrazol-5-yl]amino}carbonyl)-amino]methyl}-4-fluorobenzyl)oxy]-3-chloro-6-methyl-2-oxopyridin-1(2H)-yl}-N,4-dimethylbenzamide;
3-{3-bromo-4-[(2-{[({[3-tert-butyl-1-(4-methylphenyl)-1H-pyrazol-5-yl]amino}-carbonyl)-amino]methyl}-4-fluorobenzyl)oxy]-6-methyl-2-oxopyridin-1(2H)-yl}-4-methylbenzoic acid;
3-{4-[(2-{[({[3-tert-butyl-1-(3-methoxyphenyl)-1H-pyrazol-5-yl]amino}carbonyl)-amino]methyl}-4-fluorobenzyl)oxy]-3-chloro-6-methyl-2-oxopyridin-1(2H)-yl}-N,4-dimethyl-benzamide;
methyl 3-[4-{[2-({[({3-tert-butyl-1-[3-(2-hydroxyethoxy)-phenyl]-1H-pyrazol-5-yl}amino)-carbonyl]-amino}methyl)-benzyl]oxy}-3-chloro-6-methyl-2-oxopyridin-1(2H)-yl]-4-methylbenzoate;
3-[4-{[2-({[({3-tert-butyl-1-[3-(2-hydroxyethoxy)phenyl]-1H-pyrazol-5-yl}amino)-carbonyl]amino}methyl)-benzyl]oxy}-3-chloro-6-methyl-2-oxopyridin-1(2H)-yl]-N-(2-hydroxyethyl)-4-methylbenzamide;
3-[4-{[2-({[({3-tert-butyl-1-[3-(2-hydroxyethoxy)phenyl]-1H-pyrazol-5-yl}amino)-carbonyl]amino}methyl)benzyl]oxy}-3-chloro-6-methyl-2-oxopyridin-1(2H)-yl]-N,4-dimethylbenzamide;
3-[4-{[2-({[({3-tert-butyl-1-[3-(2-hydroxyethoxy)phenyl]-1H-pyrazol-5-yl}amino)-carbonyl]amino}methyl)benzyl]oxy}-3-chloro-6-methyl-2-oxopyridin-1(2H)-yl]-N-(2,3-dihydroxypropyl)-4-methylbenzamide;
methyl 3-{4-[(2-{[({[3-tert-butyl-1-(4-hydroxyphenyl)-1H-pyrazol-5-yl]amino}-carbonyl)amino]methyl}benzyl)oxy]-3-chloro-6-methyl-2-oxopyridin-1(2H)-yl}-4-methylbenzoate;
3-{4-[(2-{[({[3-tert-butyl-1-(4-hydroxyphenyl)-1H-pyrazol-5-yl]amino}carbonyl)-amino]methyl}benzyl)oxy]-3-chloro-6-methyl-2-oxopyridin-1(2H)-yl}-N,4-dimethyl-benzamide;
1-[3-tert-butyl-1-(3-chloro-4-hydroxyphenyl)-1H-pyrazol-5-yl]-3-[2-({[3-chloro-1-(3-methoxy-benzyl)-6-methyl-2-oxo-1,2-dihydropyridin-4-yl]oxy}methyl)-benzyl]urea;
3-{4-[(2-{[({[3-tert-butyl-1-(4-hydroxyphenyl)-1H-pyrazol-5-yl]amino}carbonyl)-amino]methyl}benzyl)oxy]-3-chloro-6-methyl-2-oxopyridin-1(2H)-yl}-N-(2-hydroxyethyl)-4-methyl-benzamide;
3-{4-[(2-{[({[3-tert-butyl-1-(4-hydroxyphenyl)-1H-pyrazol-5-yl]amino}carbonyl)-amino]methyl}benzyl)oxy]-3-chloro-6-methyl-2-oxopyridin-1(2H)-yl}-N-(2,3-dihydroxypropyl)-4-methylbenzamide;
1-[3-tert-butyl-1-(3-chloro-4-hydroxyphenyl)-1H-pyrazol-5-yl]-3-{2-[({3-chloro-6-methyl-1-[2-(methylthio)benzyl]-2-oxo-1,2-dihydropyridin-4-yl}oxy)methyl]-benzyl}urea;
1-[3-tert-butyl-1-(3-hydroxyphenyl)-1H-pyrazol-5-yl]-3-{2-[({3-chloro-6-methyl-1-[2-(methylthio)benzyl]-2-oxo-1,2-dihydropyridin-4-yl}oxy)methyl]benzyl}urea;
1-[3-tert-butyl-1-(4-chloro-3-hydroxyphenyl)-1H-pyrazol-5-yl]-3-{2-[({3-chloro-6-methyl-1-[2-(methylthio)benzyl]-2-oxo-1,2-dihydropyridin-4-yl}oxy)methyl]-benzyl}urea;
1-[3-tert-butyl-1-(4-chloro-3-hydroxyphenyl)-1H-pyrazol-5-yl]-3-[2-({[1-(2-methoxy-benzyl)-3,6-dimethyl-2-oxo-1,2-dihydropyridin-4-yl]oxy}methyl)benzyl]urea; and
3-{3-bromo-4-[(2-{[({[3-tert-butyl-1-(4-methylphenyl)-1H-pyrazol-5-yl]amino}carbonyl)-amino]methyl}-4-fluorobenzyl)oxy]-6-methyl-2-oxopyridin-1(2H)-yl}-N,4-dimethylbenzamide;
or a pharmaceutically acceptable salt thereof.

16. A pharmaceutical composition comprising a compound of claim 1, or a pharmaceutically acceptable salt thereof, and a pharmaceutically acceptable carrier material.
